(19) United States
(12) Patent Application Publication
(10) Pub. No.: US 2025/0340575 A1
(43) Pub. Date: Nov. 6, 2025

(54) 4-AMINOPYRROLO[2,1-F][1,2,4]TRIAZINES AND PREPARATION AND USES THEREOF

(71) Applicant: Biosplice Therapeutics, Inc., San Diego, CA (US)

(72) Inventors: Gopi Kumar Mittapalli, San Diego, CA (US); Chi Ching Mak, San Diego, CA (US); Brian Walter Eastman, San Diego, CA (US); Lewis Daniel Turner, San Diego, CA (US); Brian Joseph Hofilena, San Diego, CA (US); Ramkrishna Reddy Vakiti, San Diego, CA (US)

(21) Appl. No.: 19/197,446

(22) Filed: May 2, 2025

Related U.S. Application Data
(60) Provisional application No. 63/642,225, filed on May 3, 2024.

(51) Int. Cl. C07D 519/00; A61K 31/53

(52) U.S. Cl. CPC C07D 519/00; A61K 31/53

(57) ABSTRACT

4-Aminopyrrolo[2,1-f][1,2,4]triazine compounds for treating various diseases and pathologies are disclosed. More particularly, the present disclosure concerns the use of 4-aminopyrrolo[2,1-f][1,2,4]triazine compounds or analogs thereof, in the treatment of disorders characterized by overexpression of DYRK1A (e.g., cancer, Down syndrome, Alzheimer's disease, diabetes, and osteoarthritis).

### RELATED APPLICATIONS

This application claims the benefit of U.S. Provisional Application No. 63/642,225, filed May 3, 2024, which is incorporated herein by reference in its entirety.

### TECHNICAL FIELD

This disclosure relates to inhibitors of dual-specificity tyrosine phosphorylation-regulated 1A kinase, and compositions comprising the same. More particularly, it concerns the use of a 4-aminopyrrolo[2,1-f][1,2,4]triazine compound or salts or analogs thereof, in the treatment of disorders characterized by the abnormal expression and/or activity of DYRK1A (e.g., cancer, Down syndrome, Alzheimer's disease, diabetes, and osteoarthritis).

### BACKGROUND

Dual-specificity tyrosine phosphorylation-regulated kinases (DYRK1A, 1B, 2-4) comprise a family of protein kinases within the CMGC group of the eukaryotic kinome. These protein kinases are involved in multiple cellular functions, including intracellular signaling, mRNA splicing, chromatin transcription, DNA damage repair, cell survival, cell cycle control, differentiation, homocysteine/methionine/folate regulation, body temperature regulation, endocytosis, neuronal development, synaptic plasticity, etc. Abnormal expression and/or activity of some of these kinases, DYRK1A in particular, is seen in many human nervous system diseases, such as cognitive deficits associated with Down syndrome, Alzheimer's disease, and related diseases, tauopathies, dementia, Pick's disease, Parkinson's disease, and other neurodegenerative diseases, Phelan-McDermid syndrome, autism, and CDKL5 deficiency disorder. DYRKs are also involved in diabetes, abnormal folate/methionine metabolism, osteoarthritis, several solid cancers (glioblastoma, breast, and pancreatic cancers) and leukemias (acute lymphoblastic leukemia, acute megakaryoblastic leukemia), viral infections (influenza, HIV-1, HCMV, HCV, CMV, HPV), as well as infections caused by unicellular parasites (Leishmania, Trypanosoma, Plasmodium) (International Journal of Molecular Sciences (2021), 22(11), 6047). DYRK1A has also been identified as a critical stabilizer of EGFR (Cell Death & Disease (2019), 10, 282) which is a crucial factor contributing to the keratinization, cell hyperproliferation, abnormal differentiation and inflammatory infiltration during the progress of psoriasis.

### SUMMARY

The present disclosure provides methods and reagents, involving contacting a cell with an agent, such as a 4-aminopyrrolo[2,1-f][1,2,4]triazine compound, in a sufficient amount to antagonize DYRK1A activity, e.g., reduced the proliferation of head and neck squamous cell carcinoma, luminal/HER2 breast cancer (Cell (2016), 164(1-2), 293-309) or pancreatic adenocarcinoma, as well as impaired the self-renewal capacity of glioblastoma and compromised ovarian cancer spheroid cell viability (Molecular Cancer Research (2017), 15(4), 371-381).

The present disclosure also provides methods and reagents, involving contacting a cell with an agent, such as a 4-aminopyrrolo[2,1-f][1,2,4]triazine compound, in a sufficient amount to antagonize DYRK1A activity, e.g., i) to normalize prenatal and early postnatal brain development; ii) to improve cognitive function in youth and adulthood; and/or iii) to attenuate Alzheimer's-type neurodegeneration.

Some embodiments disclosed herein include DYRK1A inhibitors containing a 4-aminopyrrolo[2,1-f][1,2,4]triazine core. Other embodiments disclosed herein include pharmaceutical compositions and methods of treatment using these compounds.

One embodiment disclosed herein includes a compound having the structure of Formula I:

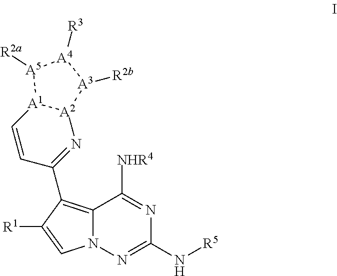

or a pharmaceutically acceptable salt thereof,
wherein,
A¹, A², A³, A⁴, and A⁵ are independently selected from the group consisting of carbon and nitrogen with the proviso that at least two of A¹, A², A³, A⁴, and A⁵ are nitrogen;
if A¹ and A³ are both nitrogen then R^{2b} is absent; if A¹ and A⁴ are both nitrogen then R³ is absent; if A¹ and A⁵ are both nitrogen then R^{2a} is absent; if A¹, A³, and A⁴ are all nitrogen then R^{2b} and R³ are absent; if A¹, A⁴, and A⁵ are all nitrogen then R^{2a} and R³ are absent; if A¹, A³, and A⁵ are all nitrogen then R^{2a} and R^{2b} are absent; if A¹, A³, A⁴, and A⁵ are all nitrogen then R^{2a}, R^{2b}, and R³ are all absent; if A² and A³ are both nitrogen then R^{2b} is absent; if A² and A⁴ are both nitrogen then R³ is absent; if A² and A⁵ are both nitrogen then R^{2a} is absent; if A², A³, and A⁴ are all nitrogen then R^{2b} and R³ are absent; if A², A⁴, and A⁵ are all nitrogen then R^{2a} and R³ are absent; if A², A³, and A⁵ are all nitrogen then R^{2a} and R^{2b} are absent; if A², A³, A⁴, and A⁵ are all nitrogen then R^{2a}, R^{2b}, and R³ are all absent;
R¹ is selected from the group consisting of H and halide;
R^{2a} is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R^{2b} is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —C(═O)N(R⁶)₂;
R³ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R⁴ is selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -carbocyclyl optionally substituted with 1-10 R⁷;
R⁵ is selected from the group consisting of -heterocyclyl optionally substituted with 1-10 R⁸ and -carbocyclyl optionally substituted with 1-12 R⁹;
each R⁶ is independently selected from the group consisting of H, unsubstituted —(C₁₋₉ alkyl), unsubstituted —(C₂₋₉ alkenyl), and unsubstituted —(C₂₋₉ alkynyl);
each R⁷ is halide;
each R⁸ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹⁰, —(C₁₋₅ alkylene)OR¹¹, —SO₂R¹², and —C(═O)R¹³, wherein the —(C₁₋₅ alkylene) is optionally substituted with 1-5 halide and/or 1-3 unsubstituted —(C₁₋₃ alkyl);
each R⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁴, and —NR¹⁵C(═O)R¹⁶;
each R¹⁰ is halide;
R¹¹ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆haloalkyl);
R¹² is selected from the group consisting of unsubstituted —(C₁₋₆alkyl), unsubstituted —(C₂₋₆ alkenyl), unsubstituted —(C₂₋₆ alkynyl), and unsubstituted —(C₁₋₆ haloalkyl);
R¹³ is selected from the group consisting of unsubstituted —(C₁₋₆ alkyl), unsubstituted —(C₂₋₆ alkenyl), unsubstituted —(C₂₋₆ alkynyl), unsubstituted —(C₁₋₆ haloalkyl), and —(C₁₋₅ alkylene)OR¹⁷, wherein the —(C₁₋₅ alkylene) is optionally substituted with 1-5 halide and/or 1-3 unsubstituted —(C₁₋₃ alkyl);
R¹⁴ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl), and —(C₁₋₅ alkylene)OR¹⁸, wherein the —(C₁₋₅ alkylene) is optionally substituted with 1-5 halide and/or 1-3 unsubstituted —(C₁₋₃ alkyl);
R¹⁵ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R¹⁶ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R¹⁷ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆haloalkyl);
R¹⁸ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆haloalkyl); and
wherein each H atom is optionally, independently replaced by ²H (D) (deuterium).

Some embodiments include stereoisomers and pharmaceutically acceptable salts of a compound of Formula (I). Some embodiments include pharmaceutically acceptable salts of a compound of Formula (I).

Some embodiments include pro-drugs of a compound of Formula (I).

Some embodiments of the present disclosure include pharmaceutical compositions comprising a compound of Formula (I) and a pharmaceutically acceptable carrier, diluent, or excipient.

Other embodiments disclosed herein include methods of inhibiting DYRK1A by administering to a patient affected by a disorder or disease in which DYRK1A overexpression is implicated, such as Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor and Stroke.

Inhibitors of DYRK1A can also be used to treat tauopathies. Tauopathies are neurodegenerative disorders characterized by the deposition of abnormal tau protein in the brain. The spectrum of tau pathologies expands beyond the traditionally discussed disease forms like Pick's disease, progressive supranuclear palsy, corticobasal degeneration, and argyrophilic grain disease. Emerging entities and pathologies include globular glial tauopathies, primary age-related tauopathy, which includes neurofibrillary tangle dementia, chronic traumatic encephalopathy (CTE), frontotemporal lobar degeneration with tau inclusions (FTLD-tau), and aging-related tau astrogliopathy. Clinical symptoms include frontotemporal dementia, corticobasal syndrome, Richardson syndrome, parkinsonism, pure akinesia with gait freezing and, rarely, motor neuron symptoms or cerebellar ataxia (Handbook of Clinical Neurology (2018), 145, 355-368 and Aging Cell (2019), 18(5), e13000).

Inhibitors of DYRK1A can also be used to treat disorders associated with abnormal folate/methionine metabolism.

Non-limiting examples of diseases which can be treated with the compounds and compositions provided herein include a variety of cancers, diabetes, psoriasis, knee osteoarthritis, tendinopathy, human immunodeficiency virus type 1 (HIV-1), human cytomegalovirus (HCMV), hepatitis C virus (HCV), and herpes simplex virus 1 (HSV-1).

Some embodiments of the present disclosure include methods to prepare compounds of Formula (I).

It is to be understood that both the foregoing general description and the following detailed description are exemplary and explanatory only and are not restrictive of the disclosure, as claimed.

### DETAILED DESCRIPTION

Provided herein are compositions and methods for inhibiting DYRK1A.

Some embodiments provided herein relate to a method for treating a disease including, but not limited to, neurological diseases or disorders, cancers, cognitive deficits, knee osteoarthritis, tendinopathy, viral infections, unicellular parasite infections, and motor deficits.

In some embodiments, non-limiting examples of a neurological disease or disorder which can be treated with the compounds and compositions provided herein include, but are not limited to, Alzheimer's disease, amyotrophic lateral sclerosis, Down Syndrome, frontotemporal dementia with Parkinsonism-17 (FTDP-17), Lewy body dementia, Parkinson's disease, Pick's disease tauopathies, and additional diseases with pronounced neurodegeneration such as autism, dementia, epilepsy, Huntington's disease, multiple sclerosis; diseases and disorders associated with acquired brain injury such as Chronic Traumatic Encephalopathy, Traumatic Brain Injury, Tumor, and Stroke.

In some embodiments, non-limiting examples of cancers which can be treated with the compounds and compositions provided herein include solid cancers (e.g., glioblastoma, ovarian, breast, and pancreatic cancers) and leukemias (e.g., acute lymphoblastic leukemia, acute megakaryoblastic leukemia, and chronic myeloid leukemia).

In some embodiments, pharmaceutical compositions are provided that are effective for treatment of a disease of an animal, e.g., a mammal, caused by DYRK1A overexpression. The composition includes a pharmaceutically acceptable carrier and a compound as described herein.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

As used herein, “alkenyl” means a straight or branched chain chemical group containing only carbon and hydrogen and containing at least one carbon-carbon double bond, such as ethenyl, 1-propenyl, 2-propenyl, 2-methyl-1-propenyl, 1-butenyl, 2-butenyl, and the like. In various embodiments, alkenyl groups can either be unsubstituted or substituted with one or more substituents. Typically, alkenyl groups will comprise 2 to 9 carbon atoms (for example, 2 to 6 carbon atoms, 2 to 4 carbon atoms, or 2 carbon atoms).

As used herein, “alkynyl” means a straight or branched chain chemical group containing only carbon and hydrogen and containing at least one carbon-carbon triple bond, such as ethynyl, 1-propynyl, 1-butynyl, 2-butynyl, and the like. In various embodiments, alkynyl groups can either be unsubstituted or substituted with one or more substituents. Typically, alkynyl groups will comprise 2 to 9 carbon atoms (for example, 2 to 6 carbon atoms, 2 to 4 carbon atoms, or 2 carbon atoms).

As used herein, “alkylene” means a bivalent branched or straight chain chemical group containing only carbon and hydrogen, such as methylene, ethylene, n-propylene, iso-propylene, n-butylene, iso-butylene, sec-butylene, tert-butylene, n-pentylene, iso-pentylene, sec-pentylene and neo-pentylene. Alkylene groups can either be unsubstituted or substituted with one or more substituents. In some embodiments, alkylene groups include 1 to 9 carbon atoms (for example, 1 to 6 carbon atoms, 1 to 4 carbon atoms, or 1 to 2 carbon atoms).

As used herein, “alkenylene” means a bivalent branched or straight chain chemical group containing only carbon and hydrogen and containing at least one carbon-carbon double bond, such as ethenylene, 1-propenylene, 2-propenylene, 2-methyl-1-propenylene, 1-butenylene, 2-butenylene, and the like. In various embodiments, alkenylene groups can either be unsubstituted or substituted with one or more substituents. Typically, alkenylene groups will comprise 2 to 9 carbon atoms (for example, 2 to 6 carbon atoms, 2 to 4 carbon atoms, or 2 carbon atoms).

As used herein, “alkynylene” means a bivalent branched or straight chain chemical group containing only carbon and hydrogen and containing at least one carbon-carbon triple bond, such as ethynylene, 1-propynylene, 1-butynylene, 2-butynylene, and the like. In various embodiments, alkynylene groups can either be unsubstituted or substituted with one or more substituents. Typically, alkynylene groups will comprise 2 to 9 carbon atoms (for example, 2 to 6 carbon atoms, 2 to 4 carbon atoms, or 2 carbon atoms).

As used herein, “alkoxy” means an alkyl-O— group in which the alkyl group is as described herein. Exemplary alkoxy groups include methoxy, ethoxy, n-propoxy, i-propoxy, n-butoxy, s-butoxy, t-butoxy, pentoxy, hexoxy and heptoxy, and also the linear or branched positional isomers thereof.

As used herein, “haloalkoxy” means a haloalkyl-O— group in which the haloalkyl group is as described herein. Exemplary haloalkoxy groups include fluoromethoxy, difluoromethoxy, trifluoromethoxy, and also the linear or branched positional isomers thereof.

As used herein, “carbocyclyl” means a cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that none of the rings in the ring system are aromatic. Carbocyclyl groups can either be unsubstituted or substituted with one or more substituents. In some embodiments, carbocyclyl groups include 3 to 10 carbon atoms, for example, 3 to 6 carbon atoms.

As used herein, “aryl” means a mono-, bi-, tri- or polycyclic group with only carbon atoms present in the ring backbone having 5 to 14 ring atoms, alternatively 5, 6, 9, or 10 ring atoms; and having 6, 10, or 14 pi electrons shared in a cyclic array; wherein at least one ring in the system is aromatic. Aryl groups can either be unsubstituted or substituted with one or more substituents. Examples of aryl include phenyl, naphthyl, tetrahydronaphthyl, 2,3-dihydro-1H-indenyl, and others. In some embodiments, the aryl is phenyl.

As used herein, the term “heteroaryl” means a mono-, bi-, tri- or polycyclic group having 5 to 14 ring atoms, alternatively 5, 6, 9, or 10 ring atoms; and having 6, 10, or 14 pi electrons shared in a cyclic array; wherein at least one ring in the system is aromatic, and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

As used herein, “halo”, “halide” or “halogen” is a chloro, bromo, fluoro, or iodo atom radical. In some embodiments, a halo is a chloro, bromo or fluoro. For example, a halide can be fluoro.

As used herein, “haloalkyl” means a hydrocarbon substituent, which is a linear or branched alkyl, alkenyl or alkynyl substituted with one or more chloro, bromo, fluoro, and/or iodo atom(s). In some embodiments, a haloalkyl is a fluoroalkyl, wherein one or more of the hydrogen atoms have been substituted by fluoro. In some embodiments, haloalkyls are 1 to 3 carbons in length (e.g., 1 to 2 carbons in length or 1 carbon in length). The term “haloalkylene” means a diradical variant of haloalkyl, and such diradicals may act as spacers between radicals, other atoms, or between a ring and another functional group.

As used herein, “heterocyclyl” means a nonaromatic cyclic ring system comprising at least one heteroatom in the ring system backbone. Heterocyclyls may include multiple fused rings such as bicyclic and spirocyclic heterocyclyls. Heterocyclyls may be substituted or unsubstituted with one or more substituents. In some embodiments, heterocycles have 3-11 members. In six membered monocyclic heterocycles, the heteroatom(s) are selected from one to three of O, N and S, and wherein when the heterocycle is five membered, it can have one or two heteroatoms selected from O, N, and S. Examples of heterocyclyl include 3-azabicyclo[3.2.1]octane, 2-azaspiro[3.5]nonanyl, 7-azaspiro[3.5]nonane, azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, 1,4-dioxaspirodecanyl, morpholinyl, thiomorpholinyl, piperazinyl, pyranyl, pyrrolidinyl, tetrahydrofuryl, tetrahydropyridinyl, octahydrocyclopenta[c]pyrrolyl, oxazinyl, 1-oxaspiro[3.5]nonanyl, 2-oxaspiro[3.5]nonanyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others. In some embodiments, the heterocyclyl is selected from azetidinyl, morpholinyl, piperazinyl, pyrrolidinyl, and tetrahydropyridinyl.

As used herein, “monocyclic heterocyclyl” means a single nonaromatic cyclic ring comprising at least one heteroatom in the ring system backbone. Heterocyclyls may be substituted or unsubstituted with one or more substituents. In some embodiments, heterocycles have 3-7 members. In six membered monocyclic heterocycles, the heteroatom(s) are selected from one to three of O, N and S, and wherein when the heterocycle is five membered, it can have one or two heteroatoms selected from O, N, and S. Examples of monocyclic heterocyclyls include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others.

As used herein, “bicyclic heterocyclyl” means a nonaromatic bicyclic ring system comprising at least one heteroatom in the ring system backbone. Bicyclic heterocyclyls may be substituted or unsubstituted with one or more substituents. In some embodiments, bicyclic heterocycles have 4-11 members with the heteroatom(s) being selected from one to five of O, N and S. Examples of bicyclic heterocyclyls include 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 3-azabicyclo[3.2.1]octane, 2-azabicyclo[2.2.2]octane, and the like.

As used herein, “spirocyclic heterocyclyl” means a nonaromatic bicyclic ring system comprising at least one heteroatom in the ring system backbone and with the rings connected through just one atom. Spirocyclic heterocyclyls may be substituted or unsubstituted with one or more substituents. In some embodiments, spirocyclic heterocycles have 5-11 members with the heteroatom(s) being selected from one to five of O, N and S. Examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 2,5-diazaspiro[3.6]decane, and the like.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substituents can include, for example, —(C₁₋₉ alkyl) optionally substituted with one or more of hydroxyl, —NH₂, —NH(C₁₋₃alkyl), and —N(C₁₋₃ alkyl)₂; —(C₁₋₉ haloalkyl); a halide; a hydroxyl; a carbonyl [such as —C(O)OR, and —C(O)R]; a thiocarbonyl [such as —C(S)OR, —C(O)SR, and —C(S)R]; —(C₁₋₉ alkoxy) optionally substituted with one or more of halide, hydroxyl, —NH₂, —NH(C₁₋₃ alkyl), and —N(C₁₋₃ alkyl)₂; —OPO(OH)₂; a phosphonate [such as —PO(OH)₂ and —PO(OR′)₂]; —OPO(OR′)R″; —NRR′; —C(O)NRR′; —C(NR)NR′R″; —C(NR′)R″; a cyano; a nitro; an azido; —SH; —S—R; —OSO₂(OR); a sulfonate [such as —SO₂(OH) and —SO₂(OR)]; —SO₂NR′R″; and —SO₂R; in which each occurrence of R, R′ and R″ are independently selected from H; —(C₁₋₉ alkyl); C₆₋₁₀ aryl optionally substituted with 1-3 R′″; 5-10 membered heteroaryl having from 1-4 heteroatoms independently selected from N, O, and S and optionally substituted with 1-3 R′″; C₃₋₇ carbocyclyl optionally substituted with 1-3 R′″; and 3-8 membered heterocyclyl having from 1-4 heteroatoms independently selected from N, O, and S and optionally substituted with 1-3 R′″; wherein each R′″ is independently selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl). In some embodiments, the substituent is selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl).

As used herein, when two groups are indicated to be “linked” or “bonded” to form a “ring”, it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions. In some embodiments, such rings have from 3-7 members, for example, 5 or 6 members.

The skilled artisan will recognize that some chemical structures described herein may be represented on paper by one or more other resonance forms; or may exist in one or more other tautomeric forms, even when kinetically, the artisan recognizes that such tautomeric forms represent only a very small portion of a sample of such compound(s). Such compounds are clearly contemplated within the scope of this disclosure, though such resonance forms or tautomers are not explicitly represented herein.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The present disclosure includes all pharmaceutically acceptable isotopically labeled compounds of Formula I wherein one or more atoms are replaced by atoms having the same atomic number, but an atomic mass or mass number different from the atomic mass or mass number which predominates in nature. Examples of isotopes suitable for inclusion in the compounds of the disclosure include, but are not limited to, isotopes of hydrogen, such as ²H (deuterium) and ³H (tritium), isotopes of carbon, such as ¹¹C, ¹³C and ¹⁴C, isotopes of chlorine, such as ³⁶Cl, isotopes of fluorine, such as ¹⁸F, isotopes of iodine, such as ¹²³I and ¹²⁵I, isotopes of nitrogen, such as ³N and ¹⁵N, isotopes of oxygen, such as ¹⁵O, ¹⁷O and ¹⁸O, isotopes of phosphorus, such as ³²P, and isotopes of sulfur, such as ³⁵S.

The term “administration” or “administering” refers to a method of providing a dosage of a compound or pharmaceutical composition to a vertebrate or invertebrate, including a mammal, a bird, a fish, or an amphibian, where the method of administration is, e.g., orally, subcutaneously, intravenously, intralymphatic, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic device. The method of administration can vary depending on various factors, e.g., the components of the pharmaceutical composition, the site of the disease, the disease involved, and the severity of the disease.

A “diagnostic” as used herein is a compound, method, system, or device that assists in the identification or characterization of a health or disease state. The diagnostic can be used in standard assays as is known in the art.

The term “mammal” is used in its usual biological sense. Thus, it specifically includes humans, cattle, horses, monkeys, dogs, cats, mice, rats, cows, sheep, pigs, goats, and non-human primates, but also includes many other species.

The terms “pharmaceutically acceptable carrier”, “pharmaceutically acceptable diluent” and “pharmaceutically acceptable excipient” include any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Brunton et al. (Eds.) (2017); Goodman and Gilman's: The Pharmacological Basis of Therapeutics, 13th Ed., The McGraw-Hill Companies.

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Many such salts are known in the art, for example, as described in WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

“Patient” as used herein, means a human or a non-human mammal, e.g., a dog, a cat, a mouse, a rat, a cow, a sheep, a pig, a goat, a non-human primate, or a bird, e.g., a chicken, as well as any other vertebrate or invertebrate. In some embodiments, the patient is a human.

A “therapeutically effective amount” of a compound as provided herein is one which is sufficient to achieve the desired physiological effect and may vary according to the nature and severity of the disease condition, and the potency of the compound. “Therapeutically effective amount” is also intended to include one or more of the compounds of Formula I in combination with one or more other agents that are effective to treat the diseases and/or conditions described herein. The combination of compounds can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age, and medical history.

A therapeutic effect relieves, to some extent, one or more of the symptoms of the disease.

“Treat,” “treatment,” or “treating,” as used herein refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing, or preventing the further development of a disorder, and/or reducing the severity of symptoms that will or are expected to develop.

“Drug-eluting” and/or controlled release as used herein refers to any and all mechanisms, e.g., diffusion, migration, permeation, and/or desorption by which the drug(s) incorporated in the drug-eluting material pass therefrom over time into the surrounding body tissue.

“Drug-eluting material” and/or controlled release material as used herein refers to any natural, synthetic, or semi-synthetic material capable of acquiring and retaining a desired shape or configuration and into which one or more drugs can be incorporated and from which incorporated drug(s) are capable of eluting over time.

“Elutable drug” as used herein refers to any drug or combination of drugs having the ability to pass over time from the drug-eluting material in which it is incorporated into the surrounding areas of the body.

### Compounds

The compounds and compositions described herein can be used to inhibit DYRK1A for treating a disorder or disease in which DYRK1A overexpression is implicated, such as in neurological diseases or disorders, cancers, cognitive deficits, knee osteoarthritis, tendinopathy, viral infections, unicellular parasite infections, and motor deficits.

Some embodiments of the present disclosure include compounds of Formula

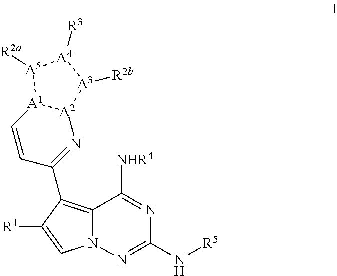

or salts, pharmaceutically acceptable salts, or prodrugs thereof.

In some embodiments of Formula I, A¹, A², A³, A⁴, and A⁵ are independently selected from the group consisting of carbon and nitrogen.

In some embodiments of Formula I, there is the proviso that at least two of A¹, A², A³, A⁴, and A⁵ are nitrogen.

In some embodiments of Formula I, if A¹ and A³ are both nitrogen then R^{2b} is absent; in some embodiments of Formula I, if A¹ and A⁴ are both nitrogen then R³ is absent; in some embodiments of Formula I, if A¹ and A⁵ are both nitrogen then R^{2a} is absent; in some embodiments of Formula I, if A¹, A³, and A⁴ are all nitrogen then R^{2b} and R³ are absent; in some embodiments of Formula I, if A¹, A⁴, and A⁵ are all nitrogen then R^{2a} and R³ are absent; in some embodiments of Formula I, if A¹, A³, and A⁵ are all nitrogen then R^{2a} and R^{2b} are absent; in some embodiments of Formula I, if A¹, A³, A⁴, and A⁵ are all nitrogen then R^{2a}, R^{2b}, and R³ are all absent; in some embodiments of Formula I, if A² and A³ are both nitrogen then R^{2b} is absent; in some embodiments of Formula I, if A² and A⁴ are both nitrogen then R³ is absent; in some embodiments of Formula I, if A² and A⁵ are both nitrogen then R^{2a} is absent; in some embodiments of Formula I, if A², A³, and A⁴ are all nitrogen then R^{2b} and R³ are absent; in some embodiments of Formula I, if A², A⁴, and A⁵ are all nitrogen then R^{2a} and R³ are absent; in some embodiments of Formula I, if A², A³, and A⁵ are all nitrogen then R^{2a} and R^{2b} are absent; and in some embodiments of Formula I, if A², A³, A⁴, and A⁵ are all nitrogen then R^{2a}, R^{2b}, and R³ are all absent.

In some embodiments of Formula I, A¹ and A³ are nitrogen, and A², A⁴, and A⁵ are carbon; in some embodiments of Formula I, A¹ and A⁴ are nitrogen, and A², A³, and A⁵ are carbon; in some embodiments of Formula I, A¹ and A⁵ are nitrogen, and A², A³, and A⁴ are carbon; in some embodiments of Formula I, A¹, A³, and A⁴ are nitrogen, and A² and A⁵ are carbon; in some embodiments of Formula I, A¹, A⁴, and A⁵ are nitrogen, and A² and A³ are carbon; in some embodiments of Formula I, A¹, A³, and A⁵ are nitrogen, and A² and A⁴ are carbon; and in some embodiments of Formula I, A¹, A³, A⁴, and A⁵ are nitrogen, and A² is carbon.

In some embodiments of Formula I, A² and A³ are nitrogen, and A¹, A⁴, and A⁵ are carbon; in some embodiments of Formula I, A² and A⁴ are nitrogen, and A¹, A³, and A⁵ are carbon; in some embodiments of Formula I, A² and A⁵ are nitrogen, and A¹, A³, and A⁴ are carbon; in some embodiments of Formula I, A², A³, and A⁴ are nitrogen, and A¹ and A⁵ are carbon; in some embodiments of Formula I, A², A⁴, and A⁵ are nitrogen, and A¹ and A³ are carbon; in some embodiments of Formula I, A², A³, and A⁵ are nitrogen, and A¹ and A⁴ are carbon; and in some embodiments of Formula I, A², A³, A⁴, and A⁵ are nitrogen, and A¹ is carbon.

In some embodiments of Formula I, A³ and A⁴ are nitrogen, and A¹, A², and A⁵ are carbon; in some embodiments of Formula I, A³ and A⁵ are nitrogen, and A¹, A², and A⁴ are carbon; in some embodiments of Formula I, A⁴ and A⁵ are nitrogen, and A¹, A², and A³ are carbon; and in some embodiments of Formula I, A³, A⁴, and A⁵ are nitrogen, and A¹ and A² are carbon.

In some embodiments of Formula I,

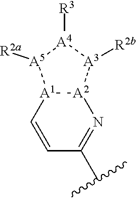

is selected from the group consisting of:

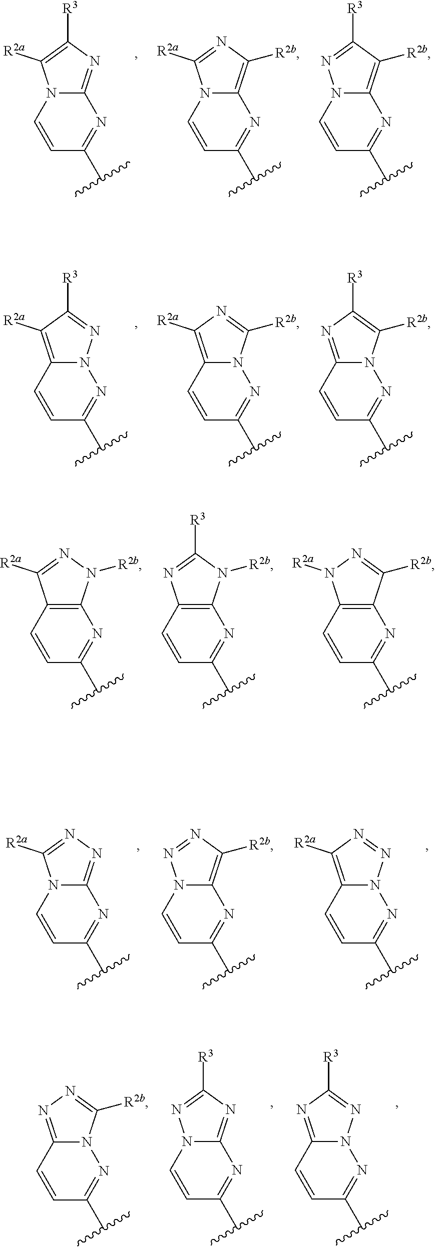

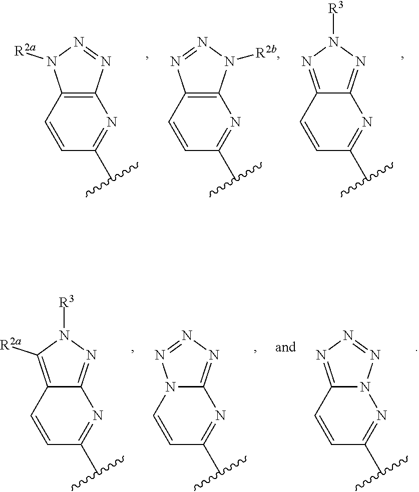

In some embodiments of Formula I,

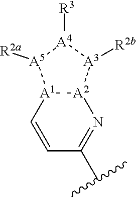

is selected from the group consisting of:

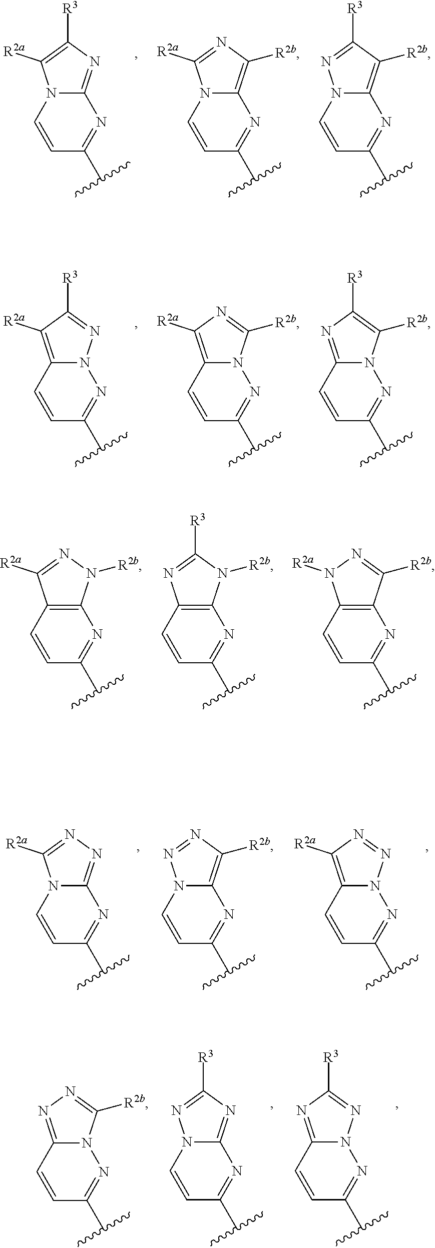

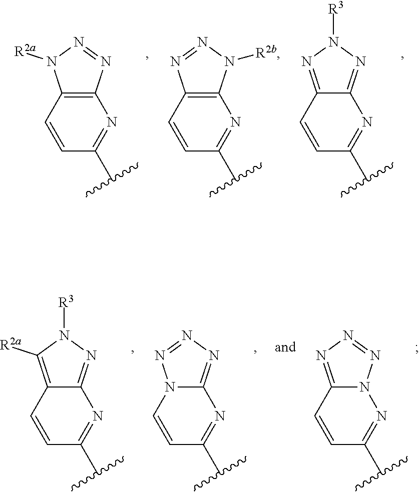

wherein R^{2a} and R³ are selected from the group consisting of H, unsubstituted —(C₁₋₅ alkyl), and unsubstituted —(C₁₋₅ haloalkyl); R^{2b} is selected from the group consisting of H, unsubstituted —(C₁₋₅ alkyl), unsubstituted —(C₁₋₅ haloalkyl), and —C(═O)NHR⁶.

In some embodiments of Formula I,

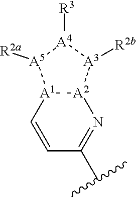

is selected from the group consisting of:

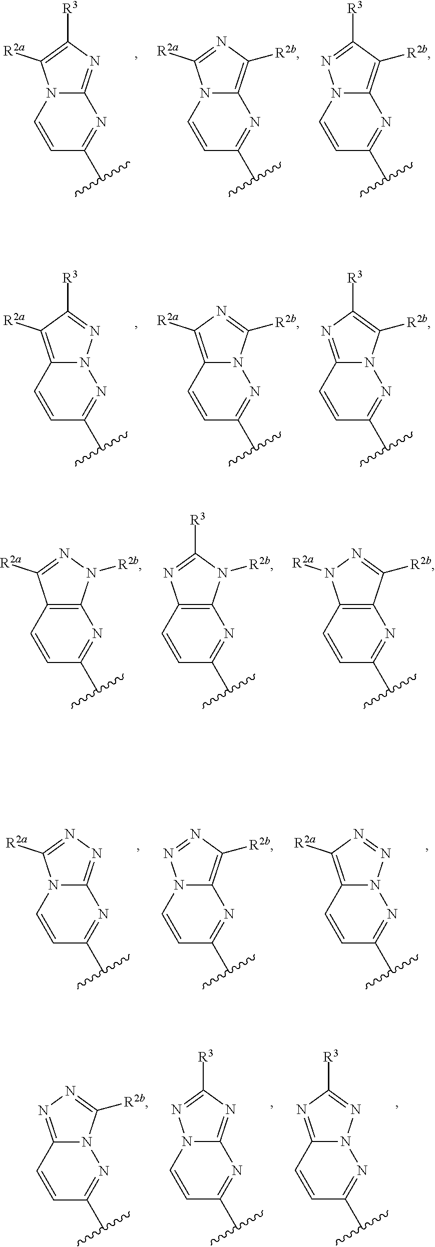

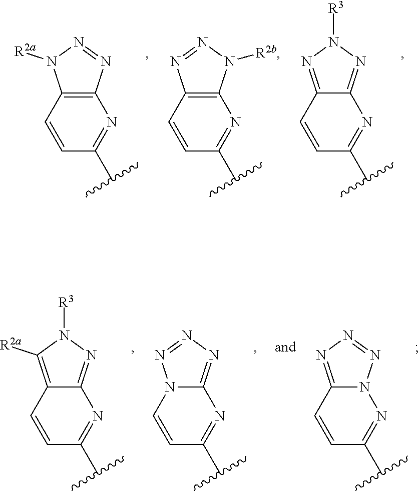

wherein R^{2a} is H; R³ is selected from the group consisting of H and Me; and R^{2b} is selected from the group consisting of unsubstituted —(C₁₋₅ alkyl), unsubstituted —(C₁₋₅ fluoroalkyl), and —C(═O)NHMe.

In some embodiments of Formula I,

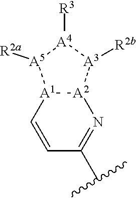

is selected from the group consisting of:

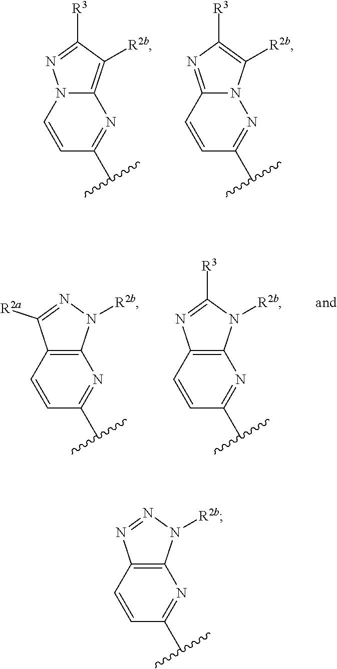

wherein R^{2a} is H; R³ is selected from the group consisting of H and Me; and R^{2b} is selected from the group consisting of unsubstituted —(C₁₋₅ alkyl), unsubstituted —(C₁₋₅ fluoroalkyl), and —C(═O)NHMe.

In some embodiments of Formula I,

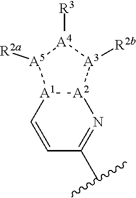

is selected from the group consisting of:

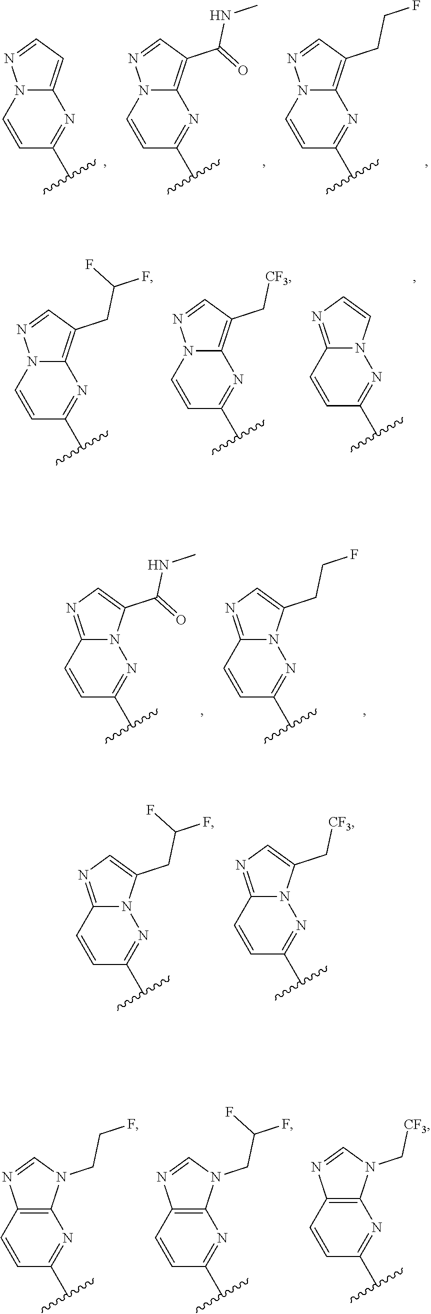

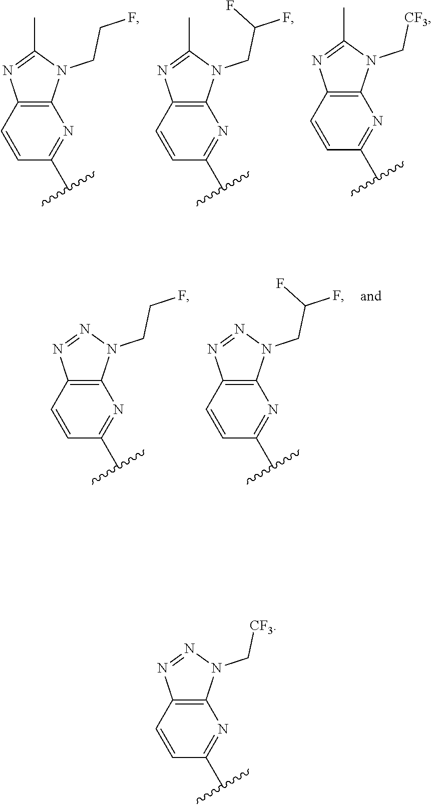 [SMILES: *c1ccc2nc(C)n(CCF)c2n1 ; *c1ccc2nc(C)n(CC(F)F)c2n1 ; *c1ccc2nc(C)n(CC(F)(F)F)c2n1 ; *c1ccc2nnn(CCF)c2n1 ; *c1ccc2nnn(CC(F)F)c2n1 ; *c1ccc2nnn(CC(F)(F)F)c2n1]

In some embodiments of Formula I, R¹ is selected from the group consisting of H and halide (e.g., F, Cl, Br, I).

In some embodiments of Formula I, R¹ is selected from the group consisting of H and F; in some embodiments of Formula I, R¹ is selected from the group consisting of H, F, and Cl; in some embodiments of Formula I, R¹ is H; in some embodiments of Formula I, R¹ is F; in some embodiments of Formula I, R¹ is Cl; in some embodiments of Formula I, R¹ is Br; in some embodiments of Formula I, R¹ is I.

In some embodiments of Formula I, R^{2a} is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, R^{2a} is selected from the group consisting of H, unsubstituted —(C₁₋₅ alkyl), and unsubstituted —(C₁₋₅ haloalkyl).

In some embodiments of Formula I, R^{2a} is selected from the group consisting of H, unsubstituted —(C₁₋₅ alkyl), and unsubstituted —(C₁₋₅ fluoroalkyl).

In some embodiments of Formula I, R^{2a} is H.

In some embodiments of Formula I, R^{2b} is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —C(═O)N(R⁶)₂.

In some embodiments of Formula I, R^{2b} is selected from the group consisting of H, unsubstituted —(C₁₋₅ alkyl), unsubstituted —(C₁₋₅ haloalkyl), and —C(═O)NHR⁶.

In some embodiments of Formula I, R^{2b} is selected from the group consisting of H, unsubstituted —(C₁₋₃ alkyl), unsubstituted —(C₁₋₃ fluoroalkyl), and —C(═O)NH(C₁₋₃ alkyl).

In some embodiments of Formula I, R^{2b} is selected from the group consisting of unsubstituted —(C₁₋₃ alkyl), unsubstituted —(C₁₋₃ fluoroalkyl), and —C(═O)NHMe.

In some embodiments of Formula I, R³ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl).

In some embodiments of Formula I, R³ is selected from the group consisting of H, unsubstituted —(C₁₋₅ alkyl), and unsubstituted —(C₁₋₅ haloalkyl).

In some embodiments of Formula I, R³ is selected from the group consisting of H, unsubstituted —(C₁₋₃ alkyl), and unsubstituted —(C₁₋₃ fluoroalkyl).

In some embodiments of Formula I, R³ is selected from the group consisting of H and Me.

In some embodiments of Formula I, R⁴ is selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -carbocyclyl optionally substituted with 1-10 (e.g., 1-9, 1-8, 1-7, 1-6, 1-5, 1-4, 1-3, 1-2, 1) R⁷.

In some embodiments of Formula I, R⁴ is selected from the group consisting of H, unsubstituted —(C₁₋₃ alkyl), unsubstituted —(C₁₋₃ haloalkyl), and -carbocyclyl optionally substituted with 1-4 (e.g., 1-3, 1-2, 1) fluorines.

In some embodiments of Formula I, R⁴ is selected from the group consisting of H, unsubstituted —(C₁₋₃ alkyl), unsubstituted —(C₁₋₃ fluoroalkyl), and unsubstituted -(3-6 membered carbocyclyl).

In some embodiments of Formula I, R⁴ is selected from the group consisting of H and unsubstituted —(C₁₋₃ alkyl).

In some embodiments of Formula I, R⁴ is unsubstituted —(C₁₋₃ alkyl).

In some embodiments of Formula I, R⁴ is Me.

In some embodiments of Formula I, R⁴ is H.

In some embodiments of Formula I, R⁵ is selected from the group consisting of -heterocyclyl optionally substituted with 1-10 (e.g., 1-9, 1-8, 1-7, 1-6, 1-5, 1-4, 1-3, 1-2, 1) R⁵, and -carbocyclyl optionally substituted with 1-12 (e.g., 1-11, 1-10, 1-9, 1-8, 1-7, 1-6, 1-5, 1-4, 1-3, 1-2, 1) R⁶.

In some embodiments of Formula I, R⁵ is selected from the group consisting of -heterocyclyl optionally substituted with 1-4 (e.g., 2-4, 1-3, 2-3, 3-4, 1-2, 1) R⁸, and -carbocyclyl optionally substituted with 1-4 (e.g., 2-4, 1-3, 2-3, 3-4, 1-2, 1) R⁹.

In some embodiments of Formula I, R⁵ is -carbocyclyl optionally substituted with 1-3 R⁹, wherein the carbocyclyl is selected from the group consisting of cyclopropyl, cyclobutyl, cyclopentyl, and cyclohexyl.

In some embodiments of Formula I, R⁵ is -carbocyclyl optionally substituted with 1-3 R⁹, wherein the carbocyclyl is selected from the group consisting of cyclobutyl and cyclohexyl.

In some embodiments of Formula I, R⁵ is selected from the group consisting of:

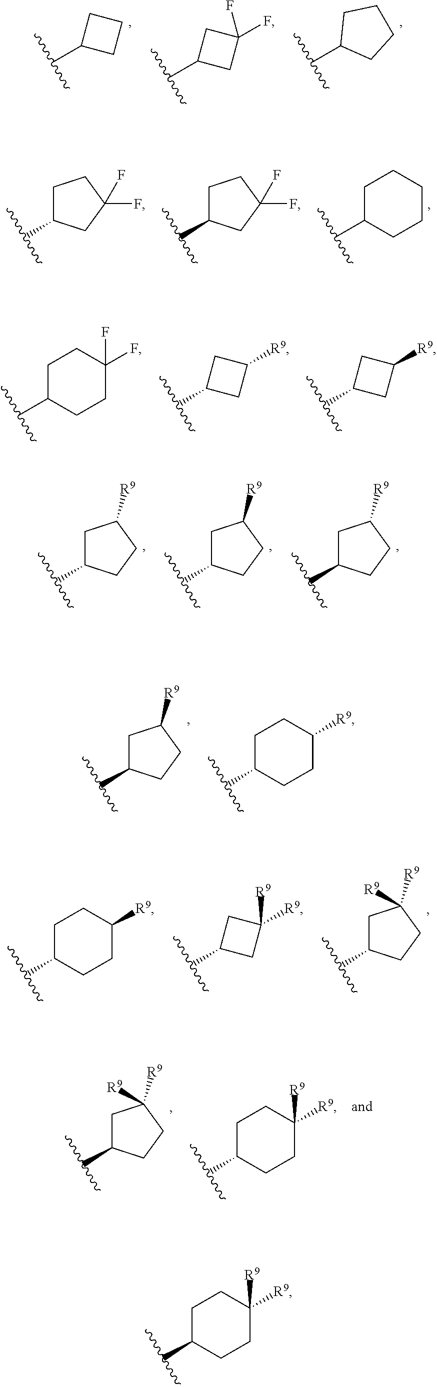

wherein each R⁹ is independently selected from the group consisting of F, Me, —CH₂F, —CHF₂, —CF₃, —OH, —OMe, —OEt, —OCF₃, —OCH₂CH₂F, —OCH₂CHF₂, —OCH₂CF₃, —OCH₂CH₂OMe, —OCH₂CH₂OH, and —NHC(═O)Me.

In some embodiments of Formula I, R⁵ is selected from the group consisting of:

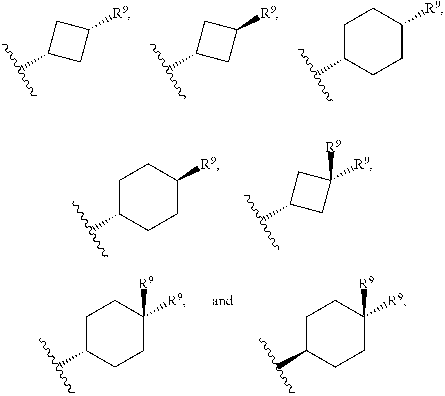

wherein each R⁹ is independently selected from the group consisting of F, Me, —OH, —OMe, and —OCH₂CH₂OH.

In some embodiments of Formula I, R⁵ is -heterocyclyl optionally substituted with 1-3 R⁸, wherein the heterocyclyl is selected from the group consisting of 3-azabicyclo[3.1.0]hexane, 3-azabicyclo[3.2.1]octane, 7-azaspiro[3.5]nonane, 2-azaspiro[3.5]nonane, oxetanyl, tetrahydrofuranyl, tetrahydropyranyl, azetidinyl, pyrrolidinyl, piperidinyl, morpholinyl, piperazinyl, 3-oxabicyclo[3.1.0]hexane, 1-oxaspiro[3.5]nonane, and oxaspiro[3.3]heptanyl.

In some embodiments of Formula I, R⁵ is selected from the group consisting of:

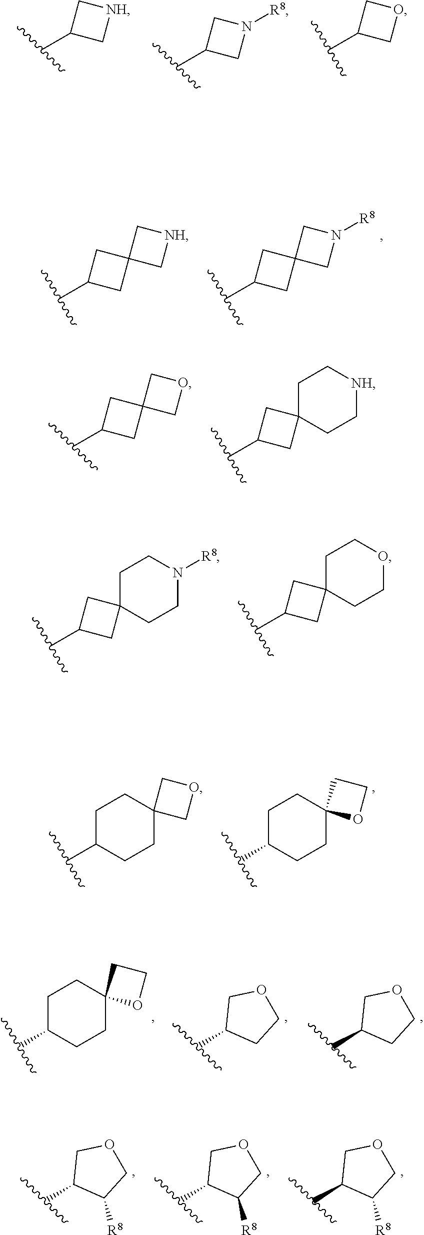

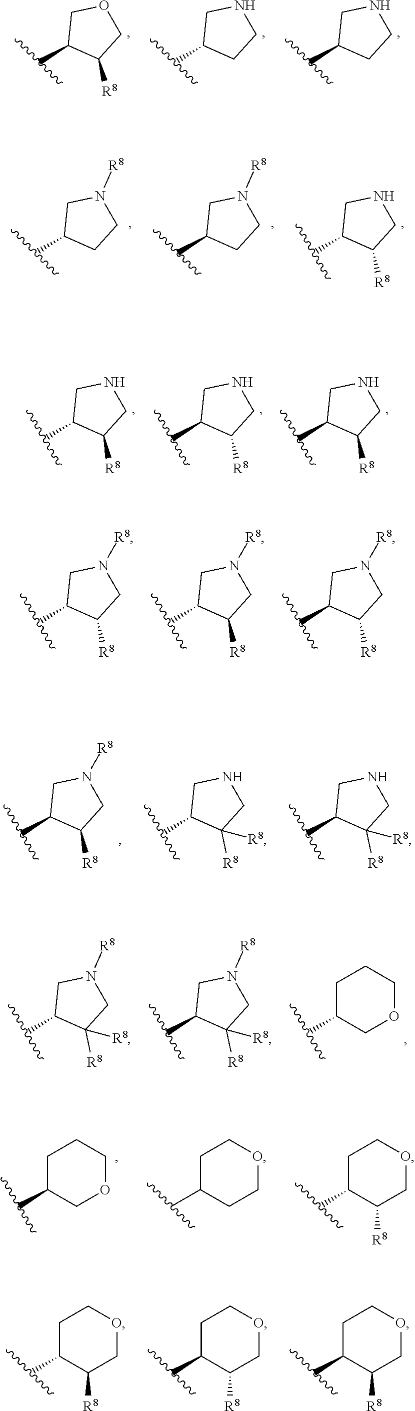

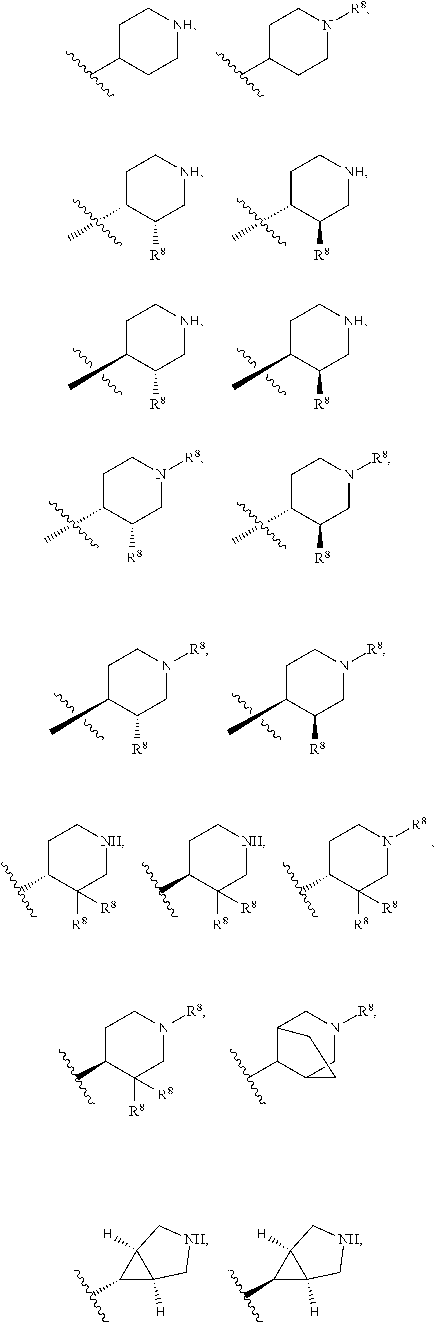

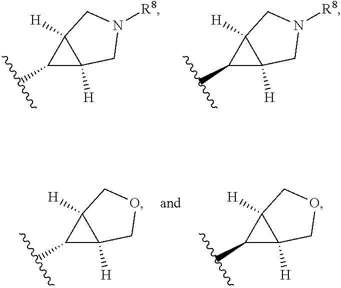

wherein each R⁸ is independently selected from the group consisting of F, Me, Et, iPr, iBu, —CH₂F, —CHF₂, —CF₃, —CH₂OH, —CH₂OMe, —OH, —OMe, —OEt, —OCD₃, —OCF₃, —CH₂CH₂F, —CH₂CHF₂, —CH₂CF₃, —CH₂CH₂CF₃—CH₂CH₂Me, —CH₂CH₂H, —C(═O)Me, —C(═O)Et, —C(═O)iPr, —C(═O)CH₂OH, —SO₂Me, and

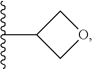 [SMILES: *C1COC1]

with the proviso that F, —OH, —OMe, —OEt, —OCD₃, and —OCF₃ are not attached to N.

In some embodiments of Formula I, R⁵ is selected from the group consisting of:

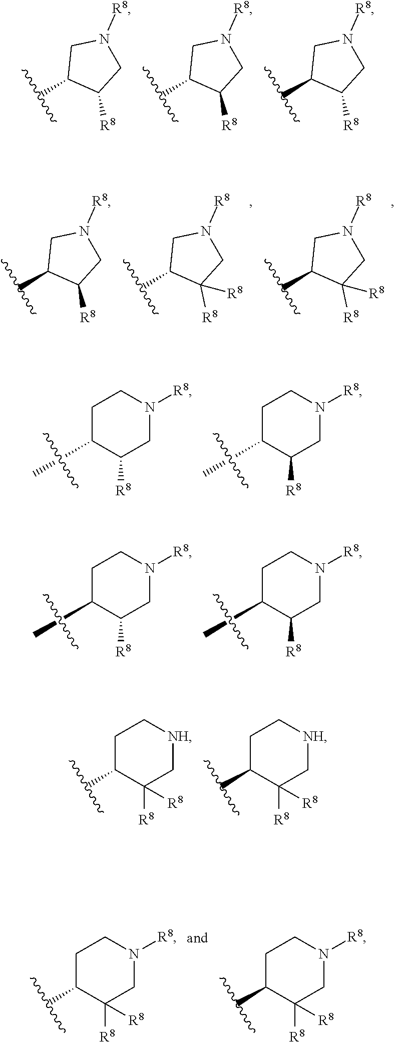

wherein each R⁸ is independently selected from the group consisting of F, Me, —CH₂CH₂F, —CH₂CH₂OMe, —C(═O)Me, —C(═O)CH₂OH, —SO₂Me, and

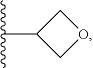 [SMILES: *C1COC1]

with the proviso that F is not attached to N.

In some embodiments of Formula I, R⁵ is selected from the group consisting of:

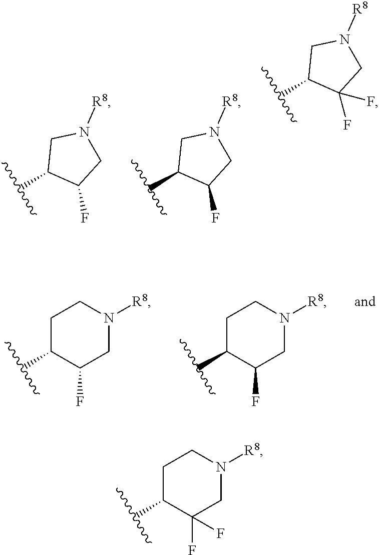

wherein each R⁸ is selected from the group consisting of Me, —C(═O)Me, —C(═O)CH₂OH, —SO₂Me, and

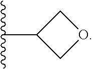 [SMILES: *C1COC1]

In some embodiments of Formula I, each R⁶ is independently selected from the group consisting of H, unsubstituted —(C₁₋₉ alkyl), unsubstituted —(C₂₋₉ alkenyl), and unsubstituted —(C₂₋₉ alkynyl).

In some embodiments of Formula I, each R⁶ is independently selected from the group consisting of H and unsubstituted —(C₁₋₅ alkyl).

In some embodiments of Formula I, each R⁶ is independently selected from the group consisting of H and Me.

In some embodiments of Formula I, each R⁷ is halide (e.g., F, Cl, Br, I).

In some embodiments of Formula I, each R⁷ is F.

In some embodiments of Formula I, each R⁸ is independently selected from the group consisting of halide (e.g., F, Cl, Br, I), unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 (e.g., 1-9, 1-8, 1-7, 1-6, 1-5, 1-4, 1-3, 1-2, 1) R¹⁰, —(C₁₋₅ alkylene)OR¹¹, —SO₂R¹², and —C(═O)R¹³, wherein the —(C₁₋₅ alkylene) is optionally substituted with 1-5 (e.g., 1-4, 1-3, 1-2, 1) halide (e.g., F, Cl, Br, I) and/or 1-3 (e.g., 1-2, 1) unsubstituted —(C₁₋₃ alkyl).

In some embodiments of Formula I, each R⁸ is independently selected from the group consisting of halide, unsubstituted —(C₁₋₅ alkyl), unsubstituted —(C₁₋₅ haloalkyl), -(3-6 membered heterocyclyl) optionally substituted with 1-3 R¹⁰, —(C₁₋₃ alkylene)OR¹¹, —SO₂R¹², and —C(═O)R¹³, wherein the —(C₁₋₃ alkylene) is unsubstituted.

In some embodiments of Formula I, each R⁸ is independently selected from the group consisting of F, unsubstituted —(C₁₋₅ alkyl), unsubstituted —(C₁₋₅ fluoroalkyl), unsubstituted -(3-4 membered heterocyclyl), —CH₂CH₂OR¹¹, —SO₂Me, and —C(═O)Me.

In some embodiments of Formula I, each R⁹ is independently selected from the group consisting of halide (e.g., F, Cl, Br, I), unsubstituted —(C₁₋₉ alkyl), unsubstituted —(C₂₋₉ alkenyl), unsubstituted-(C₂₋₉alkynyl), unsubstituted-(C₁₋₉ haloalkyl), —OR¹⁴, and —NR¹⁵C(═O)R¹⁶.

In some embodiments of Formula I, each R⁹ is independently selected from the group consisting of halide, unsubstituted —(C₁₋₅ alkyl), unsubstituted —(C₁₋₅ haloalkyl), —OR¹⁴, and —NHC(═O)R¹⁶.

In some embodiments of Formula I, each R⁹ is independently selected from the group consisting of F, unsubstituted —(C₁₋₃ alkyl), unsubstituted —(C₁₋₃ fluoroalkyl), —OH, —O(C₁₋₃ alkylene)OR¹⁸, and —NHC(═O)Me.

In some embodiments of Formula I, each R¹⁰ is halide (e.g., F, Cl, Br, I).

In some embodiments of Formula I, each R¹⁰ is F.

In some embodiments of Formula I, R¹¹ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula I, R¹¹ is selected from the group consisting of H, Me, Et, and CF₃.

In some embodiments of Formula I, R¹² is selected from the group consisting of unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula I, R¹² is selected from the group consisting of Me and Et.

In some embodiments of Formula I, R¹³ is selected from the group consisting of unsubstituted —(C₁₋₆ alkyl), unsubstituted —(C₂₋₆ alkenyl), unsubstituted —(C₂₋₆ alkynyl), unsubstituted —(C₁₋₆haloalkyl), and —(C₁₋₅ alkylene)OR¹⁷, wherein the —(C₁₋₅ alkylene) is optionally substituted with 1-5 halide and/or 1-3 unsubstituted —(C₁₋₃ alkyl).

In some embodiments of Formula I, R¹³ is selected from the group consisting of unsubstituted —(C₁₋₃ alkyl) and —CH₂OH.

In some embodiments of Formula I, R¹⁴ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆haloalkyl), and —(C₁₋₅ alkylene)OR¹⁸, wherein the —(C₁₋₅ alkylene) is optionally substituted with 1-5 halide and/or 1-3 unsubstituted —(C₁₋₃ alkyl).

In some embodiments of Formula I, R¹⁴ is selected from the group consisting of H, Me, Et, and —CH₂CH₂OH.

In some embodiments of Formula I, R¹⁵ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula I, R¹⁵ is selected from the group consisting of H and Me.

In some embodiments of Formula I, R¹⁶ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula I, R¹⁶ is Me.

In some embodiments of Formula I, R¹⁷ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula I, R¹⁷ is selected from the group consisting of H and Me.

In some embodiments of Formula I, R¹⁸ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl).

In some embodiments of Formula I, R¹⁸ is selected from the group consisting of H and Me.

In some embodiments of Formula I, each H atom is optionally, independently replaced by ²H (D) (deuterium).

Illustrative compounds of Formula I are shown in Table 1.

TABLE 1

| | |
|---|---|
| 1 | 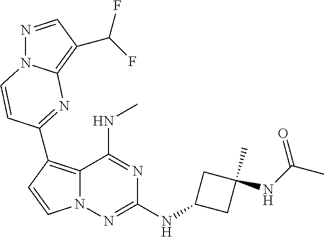 [SMILES: CNc1nc(N[C@H]2C[C@](C)(NC(C)=O)C2)nn2ccc(-c3ccn4ncc(C(F)F)c4n3)c12] |
| 2 | 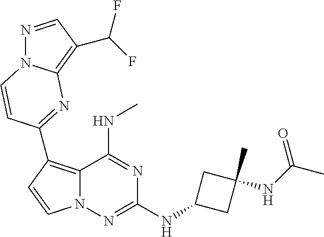 [SMILES: CNc1nc(N[C@H]2C[C@@](C)(NC(C)=O)C2)nn2ccc(-c3ccn4ncc(C(F)F)c4n3)c12] |
| 3 | 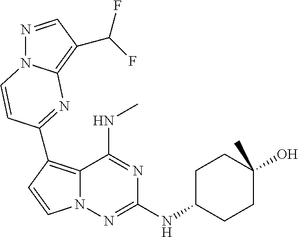 [SMILES: CNc1nc(N[C@H]2CC[C@@](C)(O)CC2)nn2ccc(-c3ccn4ncc(C(F)F)c4n3)c12] |
| 4 | 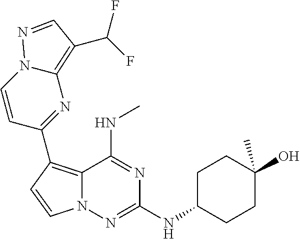 [SMILES: CNc1nc(N[C@H]2CC[C@](C)(O)CC2)nn2ccc(-c3ccn4ncc(C(F)F)c4n3)c12] |
| 5 | 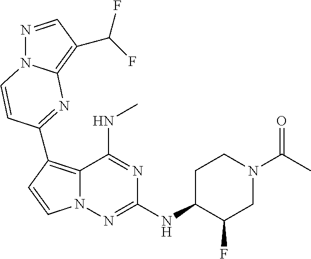 [SMILES: CNc1nc(N[C@H]2CCN(C(C)=O)C[C@H]2F)nn2ccc(-c3ccn4ncc(C(F)F)c4n3)c12] |
| 6 | 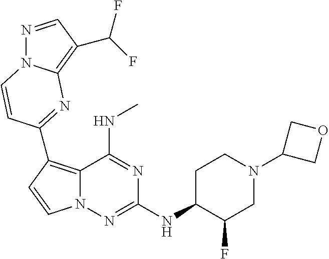 [SMILES: CNc1nc(N[C@H]2CCN(C3COC3)C[C@H]2F)nn2ccc(-c3ccn4ncc(C(F)F)c4n3)c12] |
| 7 | 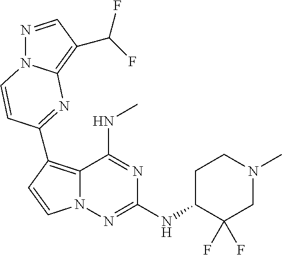 [SMILES: CNc1nc(N[C@@H]2CCN(C)CC2(F)F)nn2ccc(-c3ccn4ncc(C(F)F)c4n3)c12] |
| 8 | 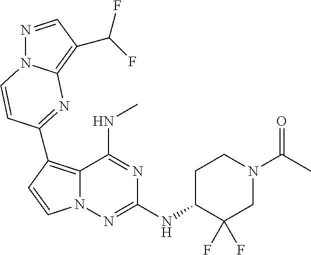 [SMILES: CNc1nc(N[C@@H]2CCN(C(C)=O)CC2(F)F)nn2ccc(-c3ccn4ncc(C(F)F)c4n3)c12] |
| 9 | 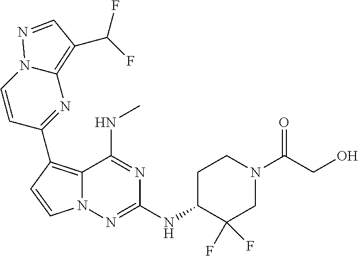 [SMILES: CNc1nc(N[C@@H]2CCN(C(=O)CO)CC2(F)F)nn2ccc(-c3ccn4ncc(C(F)F)c4n3)c12] |
| 10 | 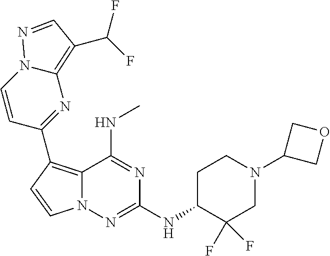 [SMILES: CNc1nc(N[C@@H]2CCN(C3COC3)CC2(F)F)nn2ccc(-c3ccn4ncc(C(F)F)c4n3)c12] |
| 11 | 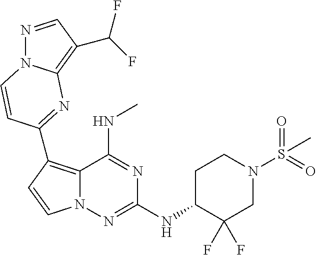 [SMILES: CNc1nc(N[C@@H]2CCN(S(C)(=O)=O)CC2(F)F)nn2ccc(-c3ccn4ncc(C(F)F)c4n3)c12] |
| 12 | 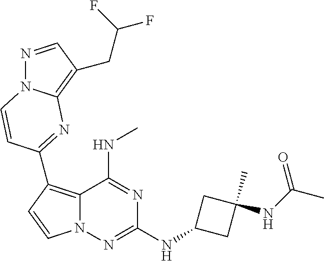 [SMILES: CNc1nc(N[C@H]2C[C@](C)(NC(C)=O)C2)nn2ccc(-c3ccn4ncc(CC(F)F)c4n3)c12] |
| 13 | 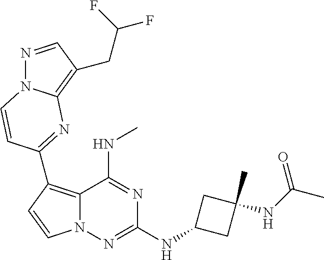 [SMILES: CNc1nc(N[C@H]2C[C@@](C)(NC(C)=O)C2)nn2ccc(-c3ccn4ncc(CC(F)F)c4n3)c12] |
| 14 | 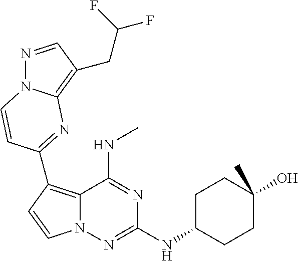 [SMILES: CNc1nc(N[C@H]2CC[C@@](C)(O)CC2)nn2ccc(-c3ccn4ncc(CC(F)F)c4n3)c12] |
| 15 | 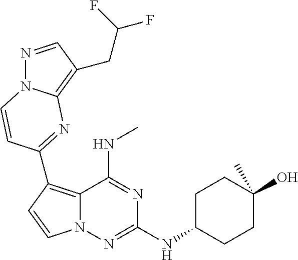 [SMILES: CNc1nc(N[C@H]2CC[C@](C)(O)CC2)nn2ccc(-c3ccn4ncc(CC(F)F)c4n3)c12] |
| 16 | 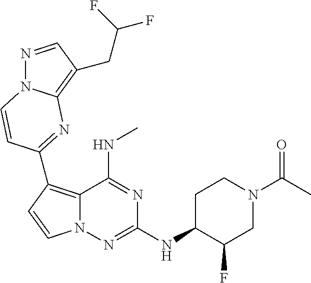 [SMILES: CNc1nc(N[C@H]2CCN(C(C)=O)C[C@H]2F)nn2ccc(-c3ccn4ncc(CC(F)F)c4n3)c12] |
| 17 | 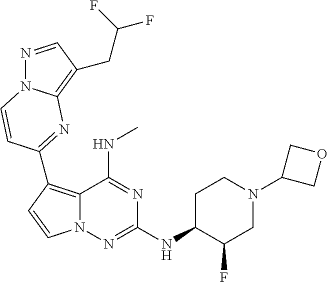 [SMILES: CNc1nc(N[C@H]2CCN(C3COC3)C[C@H]2F)nn2ccc(-c3ccn4ncc(CC(F)F)c4n3)c12] |
| 18 | 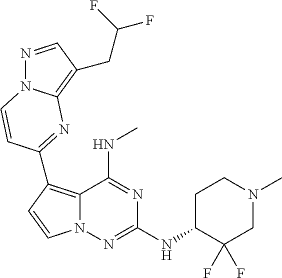 [SMILES: CNc1nc(N[C@@H]2CCN(C)CC2(F)F)nn2ccc(-c3ccn4ncc(CC(F)F)c4n3)c12] |
| 19 | 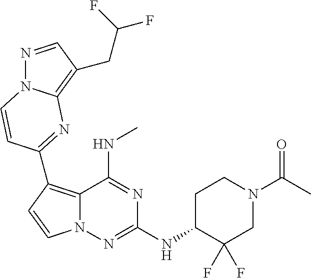 [SMILES: CNc1nc(N[C@@H]2CCN(C(C)=O)CC2(F)F)nn2ccc(-c3ccn4ncc(CC(F)F)c4n3)c12] |
| 20 | 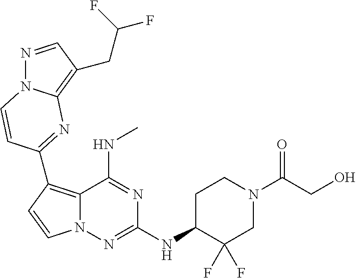 [SMILES: CNc1nc(N[C@H]2CCN(C(=O)CO)CC2(F)F)nn2ccc(-c3ccn4ncc(CC(F)F)c4n3)c12] |
| 21 | 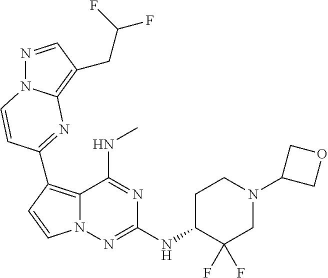 [SMILES: CNc1nc(N[C@@H]2CCN(C3COC3)CC2(F)F)nn2ccc(-c3ccn4ncc(CC(F)F)c4n3)c12] |
| 22 | 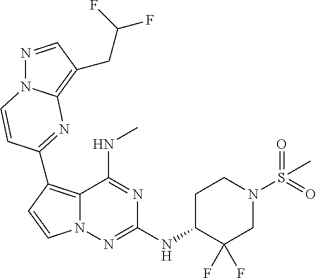 [SMILES: CNc1nc(N[C@@H]2CCN(S(C)(=O)=O)CC2(F)F)nn2ccc(-c3ccn4ncc(CC(F)F)c4n3)c12] |
| 23 | 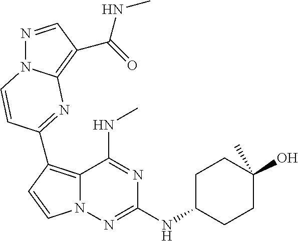 [SMILES: CNC(=O)c1cnn2ccc(-c3ccn4nc(N[C@H]5CC[C@](C)(O)CC5)nc(NC)c34)nc12] |
| 24 | 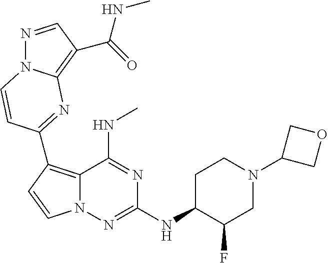 [SMILES: CNC(=O)c1cnn2ccc(-c3ccn4nc(N[C@H]5CCN(C6COC6)C[C@H]5F)nc(NC)c34)nc12] |
| 25 | 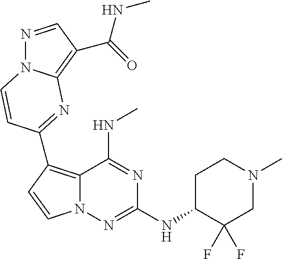 [SMILES: CNC(=O)c1cnn2ccc(-c3ccn4nc(N[C@@H]5CCN(C)CC5(F)F)nc(NC)c34)nc12] |
| 26 | 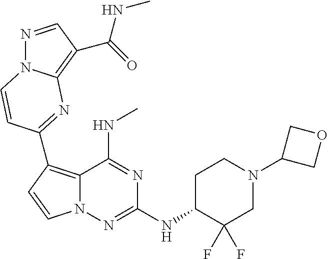 [SMILES: CNC(=O)c1cnn2ccc(-c3ccn4nc(N[C@@H]5CCN(C6COC6)CC5(F)F)nc(NC)c34)nc12] |
| 27 | 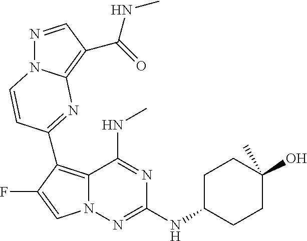 [SMILES: CNC(=O)c1cnn2ccc(-c3c(F)cn4nc(N[C@H]5CC[C@](C)(O)CC5)nc(NC)c34)nc12] |
| 28 | 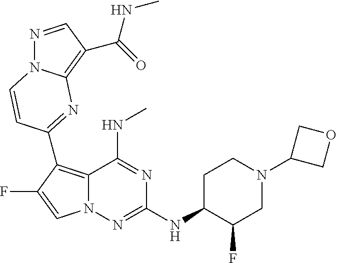 [SMILES: CNC(=O)c1cnn2ccc(-c3c(F)cn4nc(N[C@H]5CCN(C6COC6)C[C@H]5F)nc(NC)c34)nc12] |
| 29 | 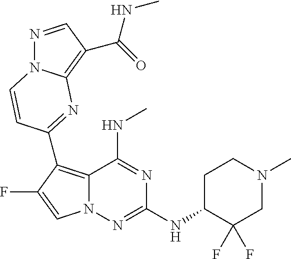 [SMILES: CNC(=O)c1cnn2ccc(-c3c(F)cn4nc(N[C@@H]5CCN(C)CC5(F)F)nc(NC)c34)nc12] |
| 30 | 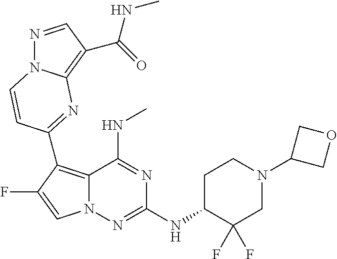 [SMILES: CNC(=O)c1cnn2ccc(-c3c(F)cn4nc(N[C@@H]5CCN(C6COC6)CC5(F)F)nc(NC)c34)nc12] |
| 31 | 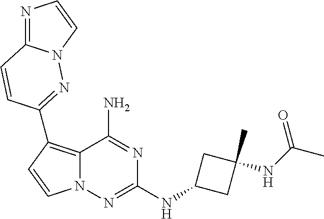 [SMILES: CC(=O)N[C@]1(C)C[C@H](Nc2nc(N)c3c(-c4ccc5nccn5n4)ccn3n2)C1] |
| 32 | 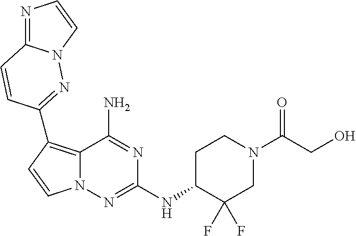 [SMILES: Nc1nc(N[C@@H]2CCN(C(=O)CO)CC2(F)F)nn2ccc(-c3ccc4nccn4n3)c12] |
| 33 | 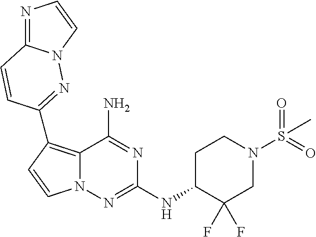 [SMILES: CS(=O)(=O)N1CC[C@@H](Nc2nc(N)c3c(-c4ccc5nccn5n4)ccn3n2)C(F)(F)C1] |
| 34 | 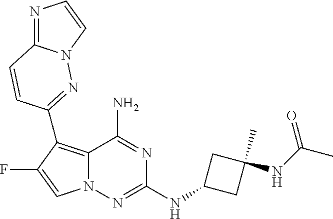 [SMILES: CC(=O)N[C@]1(C)C[C@@H](Nc2nc(N)c3c(-c4ccc5nccn5n4)c(F)cn3n2)C1] |
| 35 | 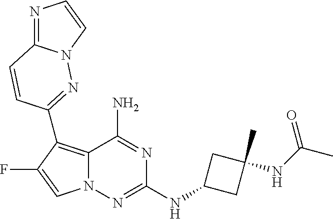 [SMILES: CC(=O)N[C@]1(C)C[C@H](Nc2nc(N)c3c(-c4ccc5nccn5n4)c(F)cn3n2)C1] |
| 36 | 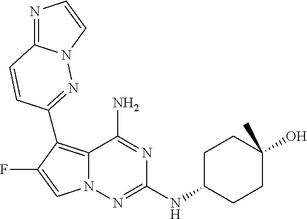 [SMILES: C[C@]1(O)CC[C@@H](Nc2nc(N)c3c(-c4ccc5nccn5n4)c(F)cn3n2)CC1] |
| 37 | 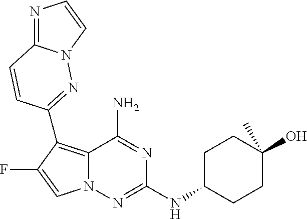 [SMILES: C[C@]1(O)CC[C@H](Nc2nc(N)c3c(-c4ccc5nccn5n4)c(F)cn3n2)CC1] |
| 38 | 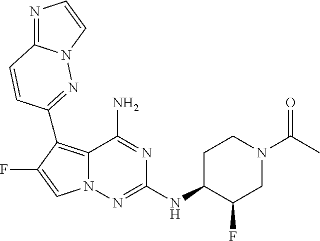 [SMILES: CC(=O)N1CC[C@H](Nc2nc(N)c3c(-c4ccc5nccn5n4)c(F)cn3n2)[C@H](F)C1] |
| 39 | 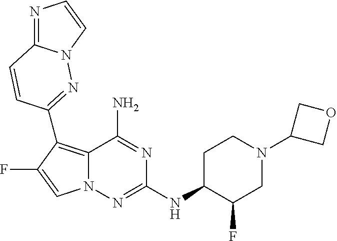 [SMILES: Nc1nc(N[C@H]2CCN(C3COC3)C[C@H]2F)nn2cc(F)c(-c3ccc4nccn4n3)c12] |
| 40 | 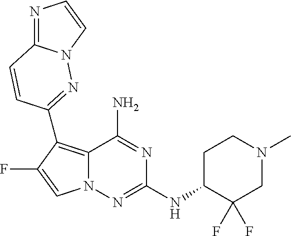 [SMILES: CN1CC[C@@H](Nc2nc(N)c3c(-c4ccc5nccn5n4)c(F)cn3n2)C(F)(F)C1] |
| 41 | 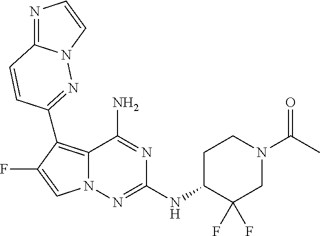 [SMILES: CC(=O)N1CC[C@@H](Nc2nc(N)c3c(-c4ccc5nccn5n4)c(F)cn3n2)C(F)(F)C1] |
| 42 | 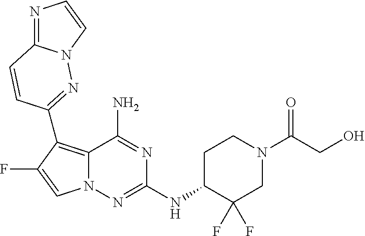 [SMILES: Nc1nc(N[C@@H]2CCN(C(=O)CO)CC2(F)F)nn2cc(F)c(-c3ccc4nccn4n3)c12] |
| 43 | 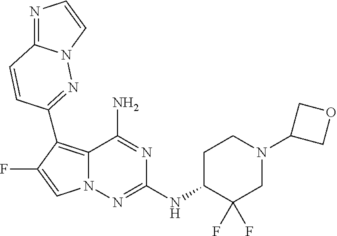 [SMILES: Nc1nc(N[C@@H]2CCN(C3COC3)CC2(F)F)nn2cc(F)c(-c3ccc4nccn4n3)c12] |
| 44 | 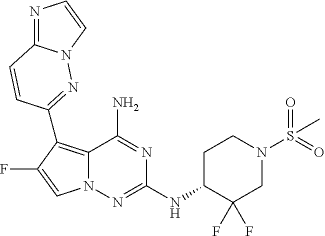 [SMILES: CS(=O)(=O)N1CC[C@@H](Nc2nc(N)c3c(-c4ccc5nccn5n4)c(F)cn3n2)C(F)(F)C1] |
| 45 | 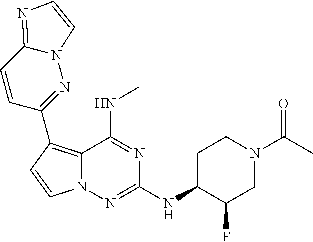 [SMILES: CNc1nc(N[C@H]2CCN(C(C)=O)C[C@H]2F)nn2ccc(-c3ccc4nccn4n3)c12] |
| 46 | 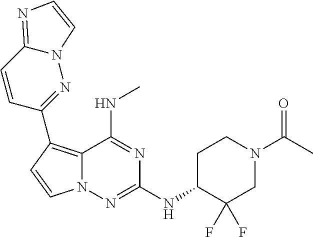 [SMILES: CNc1nc(N[C@@H]2CCN(C(C)=O)CC2(F)F)nn2ccc(-c3ccc4nccn4n3)c12] |
| 47 | 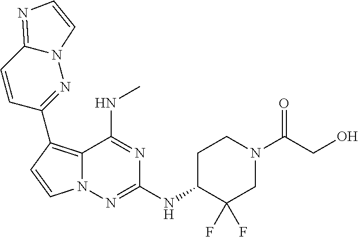 [SMILES: CNc1nc(N[C@@H]2CCN(C(=O)CO)CC2(F)F)nn2ccc(-c3ccc4nccn4n3)c12] |
| 48 | 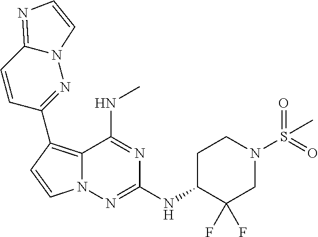 [SMILES: CNc1nc(N[C@@H]2CCN(S(C)(=O)=O)CC2(F)F)nn2ccc(-c3ccc4nccn4n3)c12] |
| 49 | 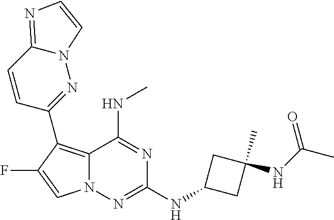 [SMILES: CNc1nc(N[C@H]2C[C@](C)(NC(C)=O)C2)nn2cc(F)c(-c3ccc4nccn4n3)c12] |
| 50 | 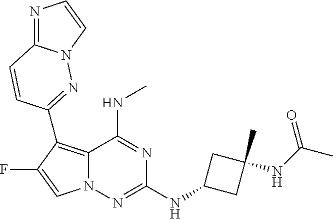 [SMILES: CNc1nc(N[C@H]2C[C@@](C)(NC(C)=O)C2)nn2cc(F)c(-c3ccc4nccn4n3)c12] |
| 51 | 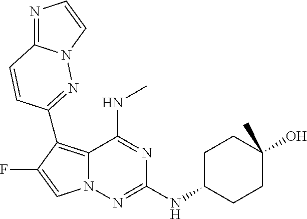 [SMILES: CNc1nc(N[C@H]2CC[C@@](C)(O)CC2)nn2cc(F)c(-c3ccc4nccn4n3)c12] |
| 52 | 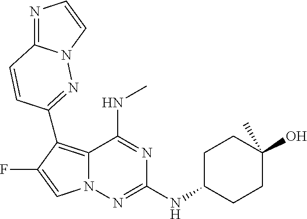 [SMILES: CNc1nc(N[C@H]2CC[C@](C)(O)CC2)nn2cc(F)c(-c3ccc4nccn4n3)c12] |
| 53 | 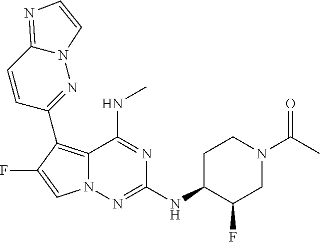 [SMILES: CNc1nc(N[C@H]2CCN(C(C)=O)C[C@H]2F)nn2cc(F)c(-c3ccc4nccn4n3)c12] |
| 54 | 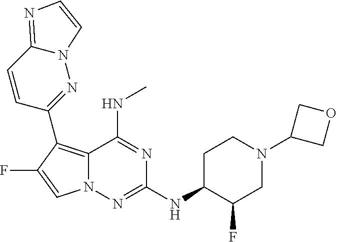 [SMILES: CNc1nc(N[C@H]2CCN(C3COC3)C[C@H]2F)nn2cc(F)c(-c3ccc4nccn4n3)c12] |
| 55 | 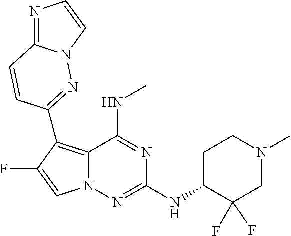 [SMILES: CNc1nc(N[C@@H]2CCN(C)CC2(F)F)nn2cc(F)c(-c3ccc4nccn4n3)c12] |
| 56 | 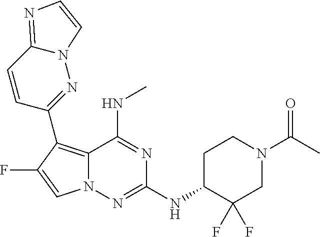 [SMILES: CNc1nc(N[C@@H]2CCN(C(C)=O)CC2(F)F)nn2cc(F)c(-c3ccc4nccn4n3)c12] |
| 57 | 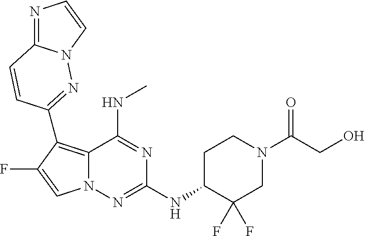 [SMILES: CNc1nc(N[C@@H]2CCN(C(=O)CO)CC2(F)F)nn2cc(F)c(-c3ccc4nccn4n3)c12] |
| 58 | 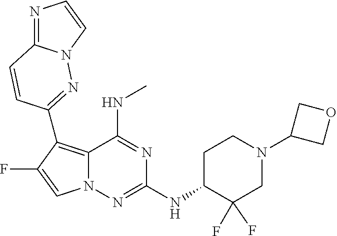 [SMILES: CNc1nc(N[C@@H]2CCN(C3COC3)CC2(F)F)nn2cc(F)c(-c3ccc4nccn4n3)c12] |
| 59 | 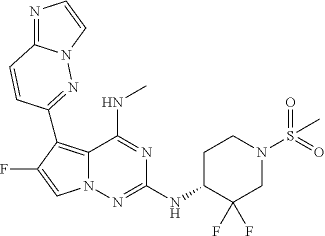 [SMILES: CNc1nc(N[C@@H]2CCN(S(C)(=O)=O)CC2(F)F)nn2cc(F)c(-c3ccc4nccn4n3)c12] |
| 60 | 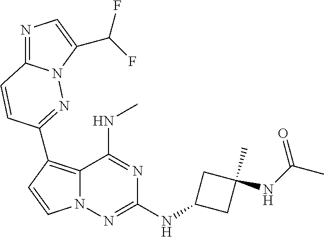 [SMILES: CNc1nc(N[C@H]2C[C@](C)(NC(C)=O)C2)nn2ccc(-c3ccc4ncc(C(F)F)n4n3)c12] |
| 61 | 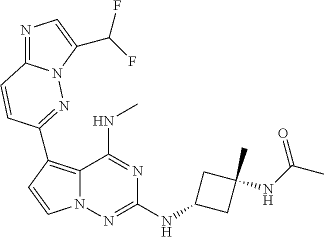 [SMILES: CNc1nc(N[C@H]2C[C@@](C)(NC(C)=O)C2)nn2ccc(-c3ccc4ncc(C(F)F)n4n3)c12] |
| 62 | 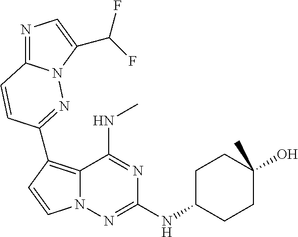 [SMILES: CNc1nc(N[C@H]2CC[C@@](C)(O)CC2)nn2ccc(-c3ccc4ncc(C(F)F)n4n3)c12] |
| 63 | 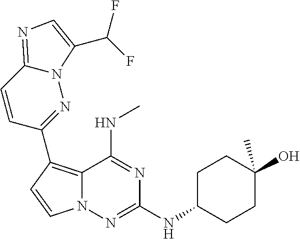 [SMILES: CNc1nc(N[C@H]2CC[C@](C)(O)CC2)nn2ccc(-c3ccc4ncc(C(F)F)n4n3)c12] |
| 64 | 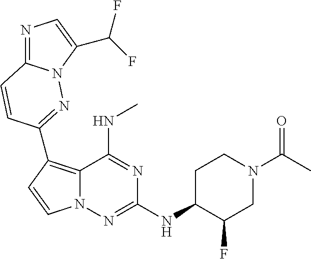 [SMILES: CNc1nc(N[C@H]2CCN(C(C)=O)C[C@H]2F)nn2ccc(-c3ccc4ncc(C(F)F)n4n3)c12] |
| 65 | 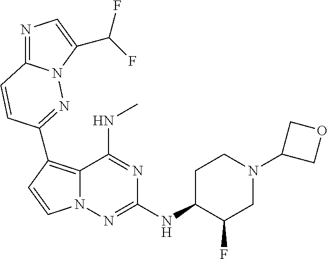 [SMILES: CNc1nc(N[C@H]2CCN(C3COC3)C[C@H]2F)nn2ccc(-c3ccc4ncc(C(F)F)n4n3)c12] |
| 66 | 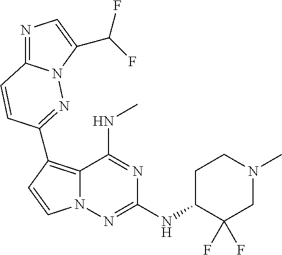 [SMILES: CNc1nc(N[C@@H]2CCN(C)CC2(F)F)nn2ccc(-c3ccc4ncc(C(F)F)n4n3)c12] |
| 67 | 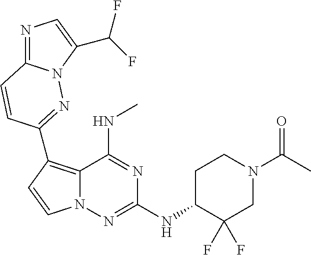 [SMILES: CNc1nc(N[C@@H]2CCN(C(C)=O)CC2(F)F)nn2ccc(-c3ccc4ncc(C(F)F)n4n3)c12] |
| 68 | 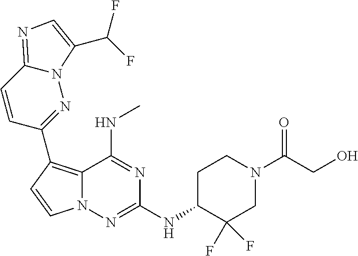 [SMILES: CNc1nc(N[C@@H]2CCN(C(=O)CO)CC2(F)F)nn2ccc(-c3ccc4ncc(C(F)F)n4n3)c12] |
| 69 | 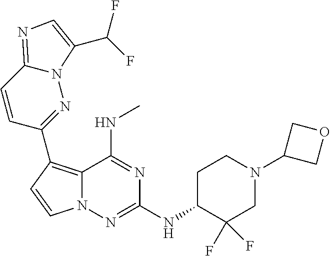 [SMILES: CNc1nc(N[C@@H]2CCN(C3COC3)CC2(F)F)nn2ccc(-c3ccc4ncc(C(F)F)n4n3)c12] |
| 70 | 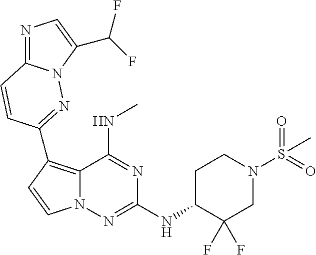 [SMILES: CNc1nc(N[C@@H]2CCN(S(C)(=O)=O)CC2(F)F)nn2ccc(-c3ccc4ncc(C(F)F)n4n3)c12] |
| 71 | 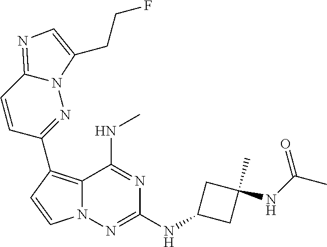 [SMILES: CNc1nc(N[C@H]2C[C@](C)(NC(C)=O)C2)nn2ccc(-c3ccc4ncc(CCF)n4n3)c12] |
| 72 | 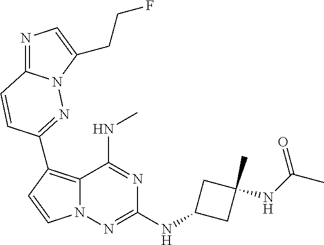 [SMILES: CNc1nc(N[C@H]2C[C@@](C)(NC(C)=O)C2)nn2ccc(-c3ccc4ncc(CCF)n4n3)c12] |
| 73 | 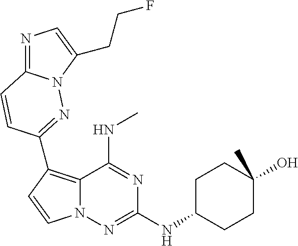 [SMILES: CNc1nc(N[C@H]2CC[C@@](C)(O)CC2)nn2ccc(-c3ccc4ncc(CCF)n4n3)c12] |
| 74 | 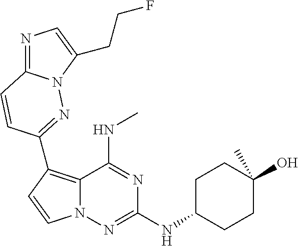 [SMILES: CNc1nc(N[C@H]2CC[C@](C)(O)CC2)nn2ccc(-c3ccc4ncc(CCF)n4n3)c12] |
| 75 | 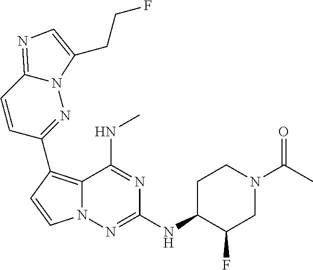 [SMILES: CNc1nc(N[C@H]2CCN(C(C)=O)C[C@H]2F)nn2ccc(-c3ccc4ncc(CCF)n4n3)c12] |
| 76 | 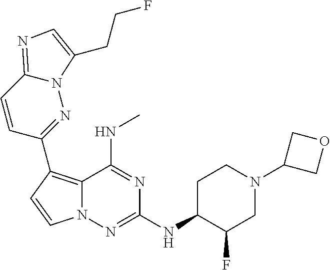 [SMILES: CNc1nc(N[C@H]2CCN(C3COC3)C[C@H]2F)nn2ccc(-c3ccc4ncc(CCF)n4n3)c12] |
| 77 | 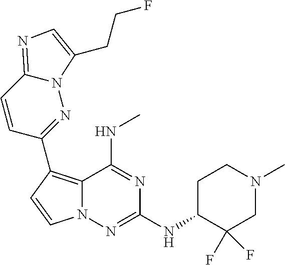 [SMILES: CNc1nc(N[C@@H]2CCN(C)CC2(F)F)nn2ccc(-c3ccc4ncc(CCF)n4n3)c12] |
| 78 | 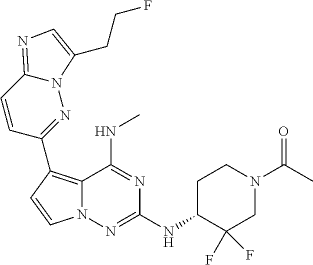 [SMILES: CNc1nc(N[C@@H]2CCN(C(C)=O)CC2(F)F)nn2ccc(-c3ccc4ncc(CCF)n4n3)c12] |
| 79 | 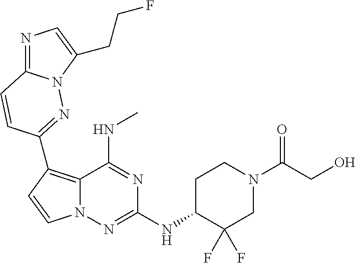 [SMILES: CNc1nc(N[C@@H]2CCN(C(=O)CO)CC2(F)F)nn2ccc(-c3ccc4ncc(CCF)n4n3)c12] |
| 80 | 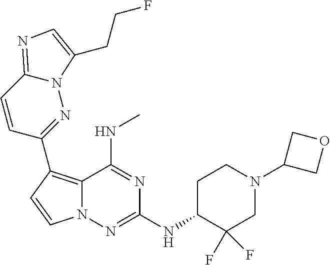 [SMILES: CNc1nc(N[C@@H]2CCN(C3COC3)CC2(F)F)nn2ccc(-c3ccc4ncc(CCF)n4n3)c12] |
| 81 | 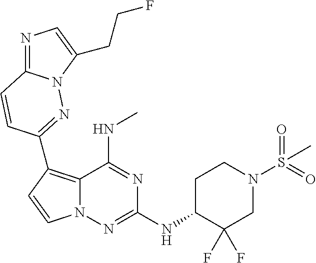 [SMILES: CNc1nc(N[C@@H]2CCN(S(C)(=O)=O)CC2(F)F)nn2ccc(-c3ccc4ncc(CCF)n4n3)c12] |
| 82 | 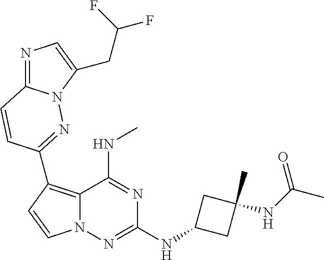 [SMILES: CNc1nc(N[C@H]2C[C@@](C)(NC(C)=O)C2)nn2ccc(-c3ccc4ncc(CC(F)F)n4n3)c12] |
| 83 | 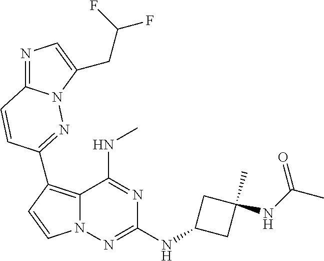 [SMILES: CNc1nc(N[C@H]2C[C@](C)(NC(C)=O)C2)nn2ccc(-c3ccc4ncc(CC(F)F)n4n3)c12] |
| 84 | 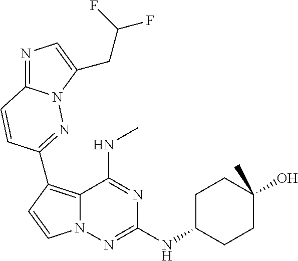 [SMILES: CNc1nc(N[C@H]2CC[C@@](C)(O)CC2)nn2ccc(-c3ccc4ncc(CC(F)F)n4n3)c12] |
| 85 | 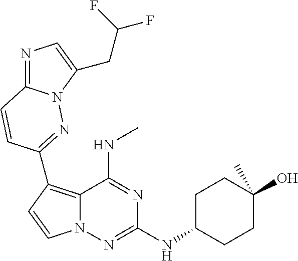 [SMILES: CNc1nc(N[C@H]2CC[C@](C)(O)CC2)nn2ccc(-c3ccc4ncc(CC(F)F)n4n3)c12] |
| 86 | 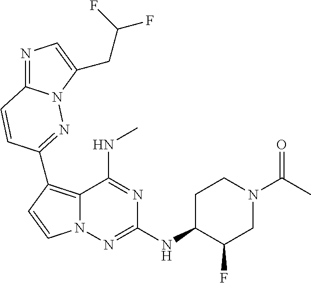 [SMILES: CNc1nc(N[C@H]2CCN(C(C)=O)C[C@H]2F)nn2ccc(-c3ccc4ncc(CC(F)F)n4n3)c12] |
| 87 | 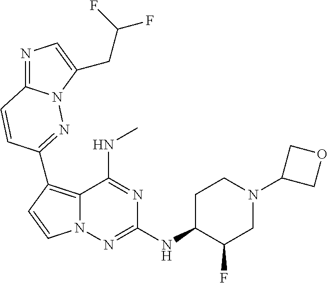 [SMILES: CNc1nc(N[C@H]2CCN(C3COC3)C[C@H]2F)nn2ccc(-c3ccc4ncc(CC(F)F)n4n3)c12] |
| 88 | 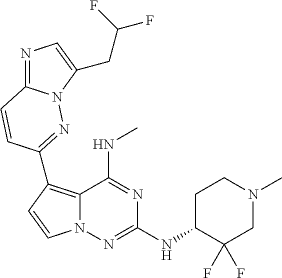 [SMILES: CNc1nc(N[C@@H]2CCN(C)CC2(F)F)nn2ccc(-c3ccc4ncc(CC(F)F)n4n3)c12] |
| 89 | 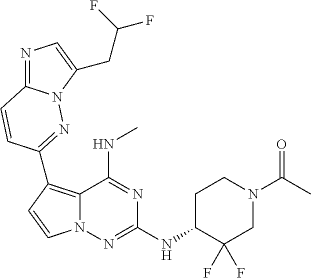 [SMILES: CNc1nc(N[C@@H]2CCN(C(C)=O)CC2(F)F)nn2ccc(-c3ccc4ncc(CC(F)F)n4n3)c12] |
| 90 | 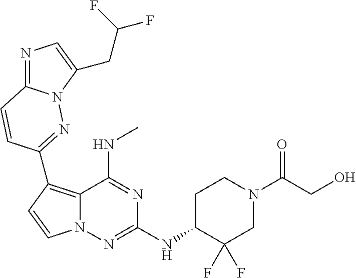 [SMILES: CNc1nc(N[C@@H]2CCN(C(=O)CO)CC2(F)F)nn2ccc(-c3ccc4ncc(CC(F)F)n4n3)c12] |
| 91 | 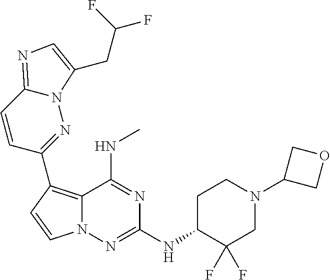 [SMILES: CNc1nc(N[C@@H]2CCN(C3COC3)CC2(F)F)nn2ccc(-c3ccc4ncc(CC(F)F)n4n3)c12] |
| 92 | 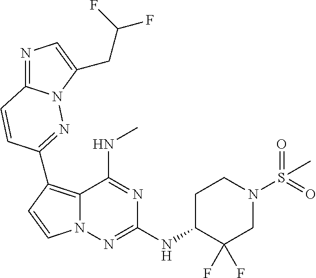 [SMILES: CNc1nc(N[C@@H]2CCN(S(C)(=O)=O)CC2(F)F)nn2ccc(-c3ccc4ncc(CC(F)F)n4n3)c12] |
| 93 | 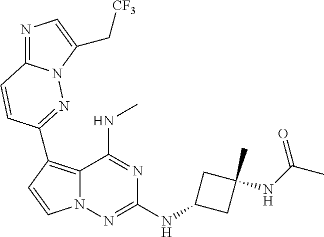 [SMILES: CNc1nc(N[C@H]2C[C@@](C)(NC(C)=O)C2)nn2ccc(-c3ccc4ncc(CC(F)(F)F)n4n3)c12] |
| 94 | 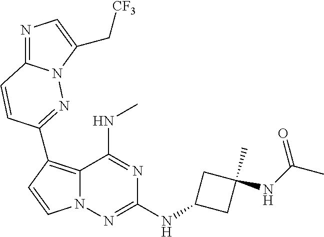 [SMILES: CNc1nc(N[C@H]2C[C@](C)(NC(C)=O)C2)nn2ccc(-c3ccc4ncc(CC(F)(F)F)n4n3)c12] |
| 95 | 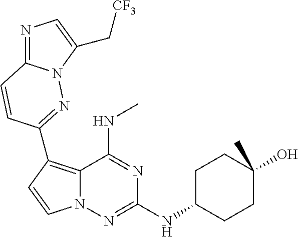 [SMILES: CNc1nc(N[C@H]2CC[C@@](C)(O)CC2)nn2ccc(-c3ccc4ncc(CC(F)(F)F)n4n3)c12] |
| 96 | 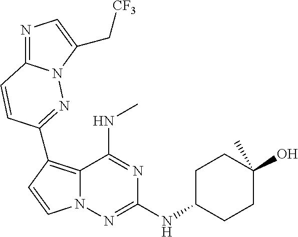 [SMILES: CNc1nc(N[C@H]2CC[C@](C)(O)CC2)nn2ccc(-c3ccc4ncc(CC(F)(F)F)n4n3)c12] |
| 97 | 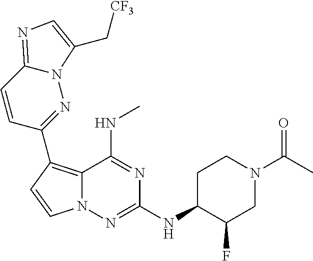 [SMILES: CNc1nc(N[C@H]2CCN(C(C)=O)C[C@H]2F)nn2ccc(-c3ccc4ncc(CC(F)(F)F)n4n3)c12] |
| 98 | 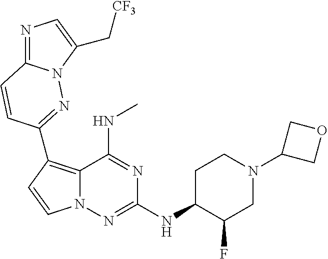 [SMILES: CNc1nc(N[C@H]2CCN(C3COC3)C[C@H]2F)nn2ccc(-c3ccc4ncc(CC(F)(F)F)n4n3)c12] |
| 99 | 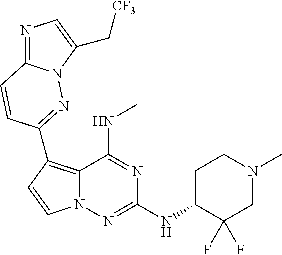 [SMILES: CNc1nc(N[C@@H]2CCN(C)CC2(F)F)nn2ccc(-c3ccc4ncc(CC(F)(F)F)n4n3)c12] |
| 100 | 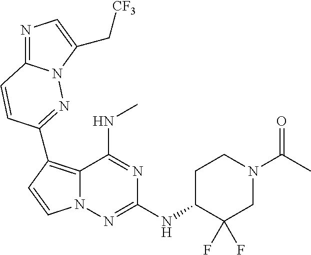 [SMILES: CNc1nc(N[C@@H]2CCN(C(C)=O)CC2(F)F)nn2ccc(-c3ccc4ncc(CC(F)(F)F)n4n3)c12] |
| 101 | 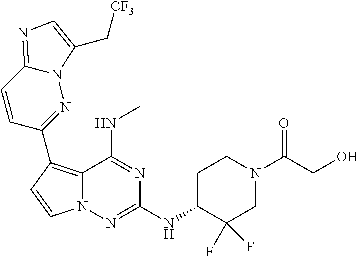 [SMILES: CNc1nc(N[C@@H]2CCN(C(=O)CO)CC2(F)F)nn2ccc(-c3ccc4ncc(CC(F)(F)F)n4n3)c12] |
| 102 | 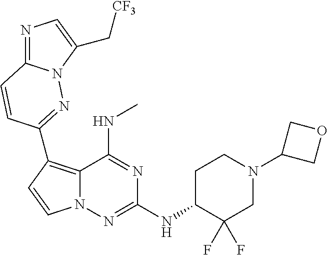 [SMILES: CNc1nc(N[C@@H]2CCN(C3COC3)CC2(F)F)nn2ccc(-c3ccc4ncc(CC(F)(F)F)n4n3)c12] |
| 103 | 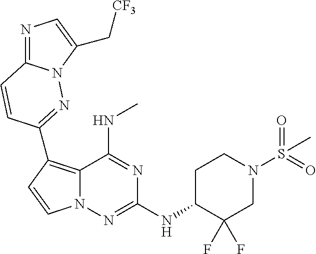 [SMILES: CNc1nc(N[C@@H]2CCN(S(C)(=O)=O)CC2(F)F)nn2ccc(-c3ccc4ncc(CC(F)(F)F)n4n3)c12] |
| 104 | 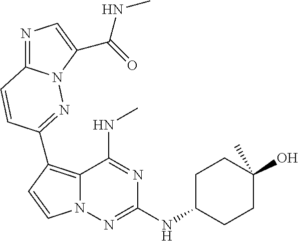 [SMILES: CNC(=O)c1cnc2ccc(-c3ccn4nc(N[C@H]5CC[C@](C)(O)CC5)nc(NC)c34)nn12] |
| 105 | 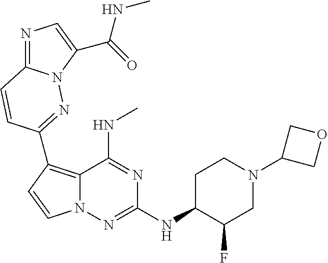 [SMILES: CNC(=O)c1cnc2ccc(-c3ccn4nc(N[C@H]5CCN(C6COC6)C[C@H]5F)nc(NC)c34)nn12] |
| 106 | 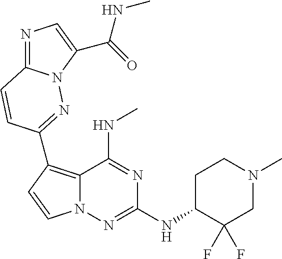 [SMILES: CNC(=O)c1cnc2ccc(-c3ccn4nc(N[C@@H]5CCN(C)CC5(F)F)nc(NC)c34)nn12] |
| 107 | 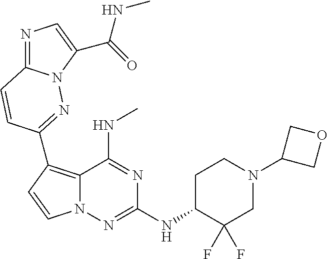 [SMILES: CNC(=O)c1cnc2ccc(-c3ccn4nc(N[C@@H]5CCN(C6COC6)CC5(F)F)nc(NC)c34)nn12] |
| 108 | 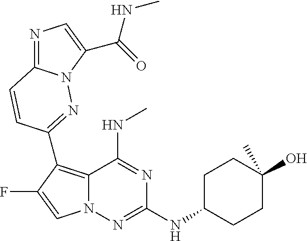 [SMILES: CNC(=O)c1cnc2ccc(-c3c(F)cn4nc(N[C@H]5CC[C@](C)(O)CC5)nc(NC)c34)nn12] |
| 109 | 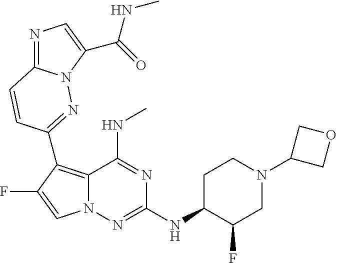 [SMILES: CNC(=O)c1cnc2ccc(-c3c(F)cn4nc(N[C@H]5CCN(C6COC6)C[C@H]5F)nc(NC)c34)nn12] |
| 110 | 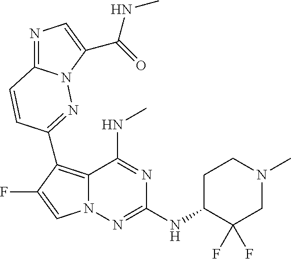 [SMILES: CNC(=O)c1cnc2ccc(-c3c(F)cn4nc(N[C@@H]5CCN(C)CC5(F)F)nc(NC)c34)nn12] |
| 111 | 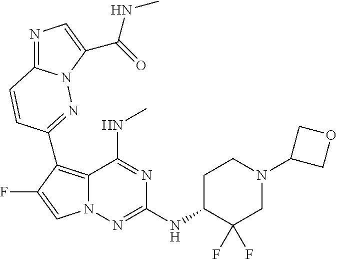 [SMILES: CNC(=O)c1cnc2ccc(-c3c(F)cn4nc(N[C@@H]5CCN(C6COC6)CC5(F)F)nc(NC)c34)nn12] |
| 112 | 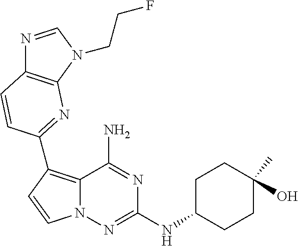 [SMILES: C[C@]1(O)CC[C@H](Nc2nc(N)c3c(-c4ccc5ncn(CCF)c5n4)ccn3n2)CC1] |
| 113 | 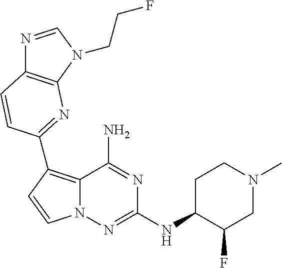 [SMILES: CN1CC[C@H](Nc2nc(N)c3c(-c4ccc5ncn(CCF)c5n4)ccn3n2)[C@H](F)C1] |
| 114 | 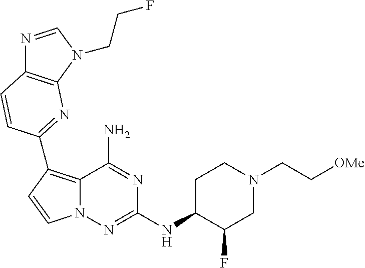 [SMILES: COCCN1CC[C@H](Nc2nc(N)c3c(-c4ccc5ncn(CCF)c5n4)ccn3n2)[C@H](F)C1] |
| 115 | 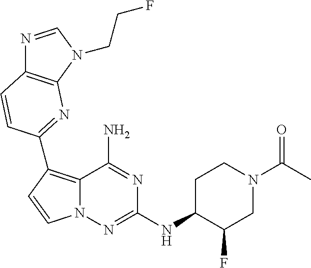 [SMILES: CC(=O)N1CC[C@H](Nc2nc(N)c3c(-c4ccc5ncn(CCF)c5n4)ccn3n2)[C@H](F)C1] |
| 116 | 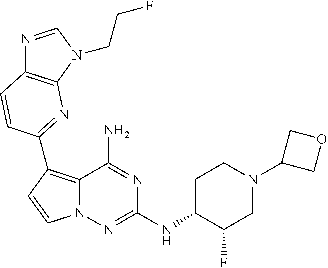 [SMILES: Nc1nc(N[C@@H]2CCN(C3COC3)C[C@@H]2F)nn2ccc(-c3ccc4ncn(CCF)c4n3)c12] |
| 117 | 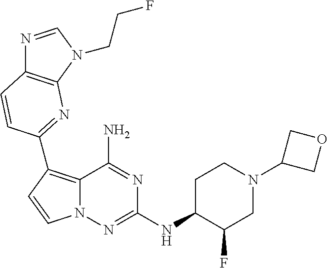 [SMILES: Nc1nc(N[C@H]2CCN(C3COC3)C[C@H]2F)nn2ccc(-c3ccc4ncn(CCF)c4n3)c12] |
| 118 | 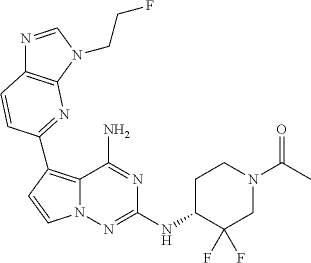 [SMILES: CC(=O)N1CC[C@@H](Nc2nc(N)c3c(-c4ccc5ncn(CCF)c5n4)ccn3n2)C(F)(F)C1] |
| 119 | 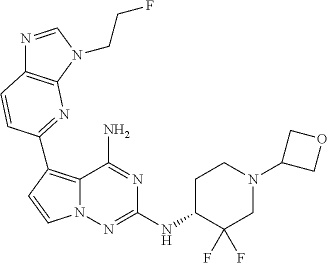 [SMILES: Nc1nc(N[C@@H]2CCN(C3COC3)CC2(F)F)nn2ccc(-c3ccc4ncn(CCF)c4n3)c12] |
| 120 | 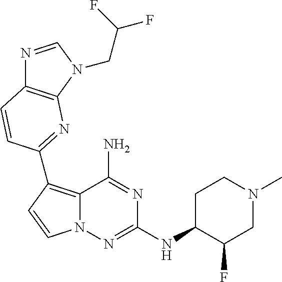 [SMILES: CN1CC[C@H](Nc2nc(N)c3c(-c4ccc5ncn(CC(F)F)c5n4)ccn3n2)[C@H](F)C1] |
| 121 | 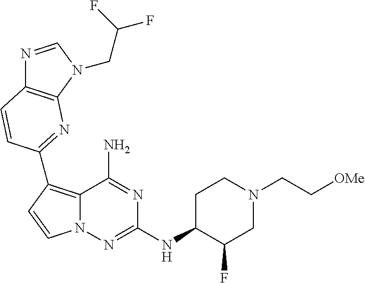 [SMILES: COCCN1CC[C@H](Nc2nc(N)c3c(-c4ccc5ncn(CC(F)F)c5n4)ccn3n2)[C@H](F)C1] |
| 122 | 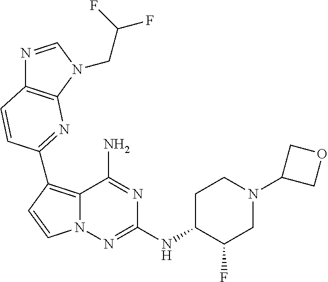 [SMILES: Nc1nc(N[C@@H]2CCN(C3COC3)C[C@@H]2F)nn2ccc(-c3ccc4ncn(CC(F)F)c4n3)c12] |
| 123 | 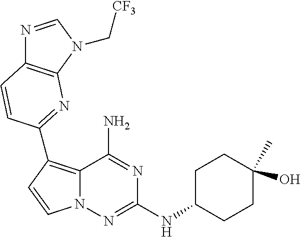 [SMILES: C[C@]1(O)CC[C@H](Nc2nc(N)c3c(-c4ccc5ncn(CC(F)(F)F)c5n4)ccn3n2)CC1] |
| 124 | 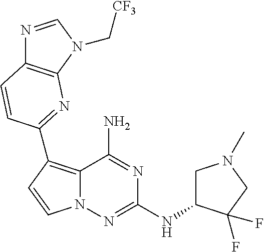 [SMILES: CN1C[C@@H](Nc2nc(N)c3c(-c4ccc5ncn(CC(F)(F)F)c5n4)ccn3n2)C(F)(F)C1] |
| 125 | 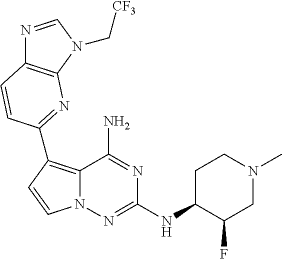 [SMILES: CN1CC[C@H](Nc2nc(N)c3c(-c4ccc5ncn(CC(F)(F)F)c5n4)ccn3n2)[C@H](F)C1] |
| 126 | 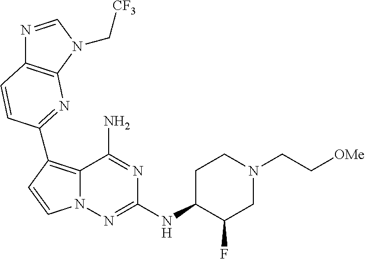 [SMILES: COCCN1CC[C@H](Nc2nc(N)c3c(-c4ccc5ncn(CC(F)(F)F)c5n4)ccn3n2)[C@H](F)C1] |
| 127 | 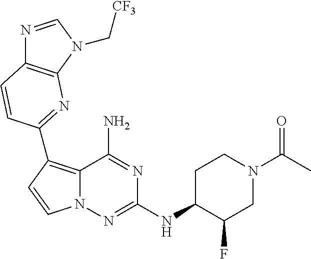 [SMILES: CC(=O)N1CC[C@H](Nc2nc(N)c3c(-c4ccc5ncn(CC(F)(F)F)c5n4)ccn3n2)[C@H](F)C1] |
| 128 | 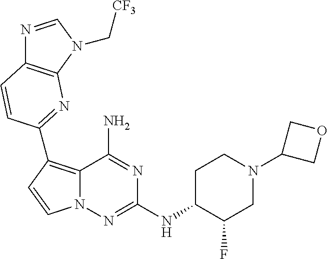 [SMILES: Nc1nc(N[C@@H]2CCN(C3COC3)C[C@@H]2F)nn2ccc(-c3ccc4ncn(CC(F)(F)F)c4n3)c12] |
| 129 | 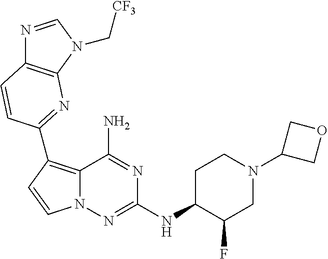 [SMILES: Nc1nc(N[C@H]2CCN(C3COC3)C[C@H]2F)nn2ccc(-c3ccc4ncn(CC(F)(F)F)c4n3)c12] |
| 130 | 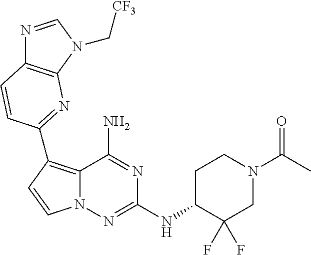 [SMILES: CC(=O)N1CC[C@@H](Nc2nc(N)c3c(-c4ccc5ncn(CC(F)(F)F)c5n4)ccn3n2)C(F)(F)C1] |
| 131 | 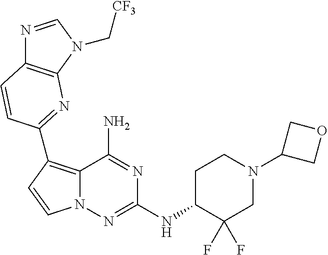 [SMILES: Nc1nc(N[C@@H]2CCN(C3COC3)CC2(F)F)nn2ccc(-c3ccc4ncn(CC(F)(F)F)c4n3)c12] |
| 132 | 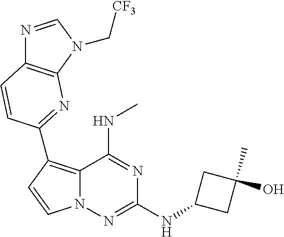 [SMILES: CNc1nc(N[C@H]2C[C@](C)(O)C2)nn2ccc(-c3ccc4ncn(CC(F)(F)F)c4n3)c12] |
| 133 | 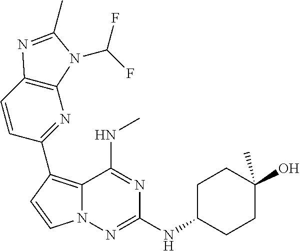 [SMILES: CNc1nc(N[C@H]2CC[C@](C)(O)CC2)nn2ccc(-c3ccc4nc(C)n(C(F)F)c4n3)c12] |
| 134 | 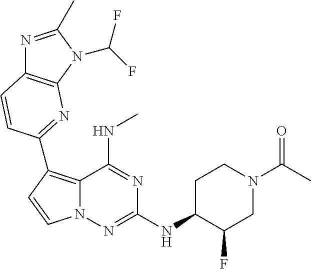 [SMILES: CNc1nc(N[C@H]2CCN(C(C)=O)C[C@H]2F)nn2ccc(-c3ccc4nc(C)n(C(F)F)c4n3)c12] |
| 135 | 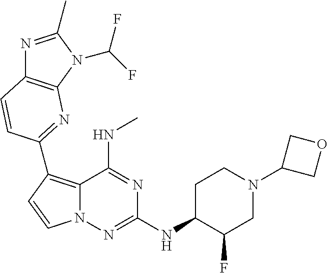 [SMILES: CNc1nc(N[C@H]2CCN(C3COC3)C[C@H]2F)nn2ccc(-c3ccc4nc(C)n(C(F)F)c4n3)c12] |
| 136 | 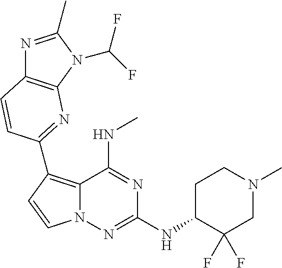 [SMILES: CNc1nc(N[C@@H]2CCN(C)CC2(F)F)nn2ccc(-c3ccc4nc(C)n(C(F)F)c4n3)c12] |
| 137 | 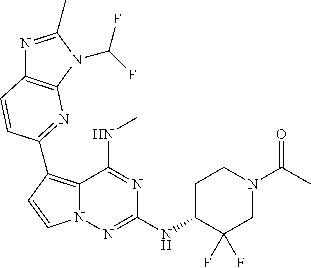 [SMILES: CNc1nc(N[C@@H]2CCN(C(C)=O)CC2(F)F)nn2ccc(-c3ccc4nc(C)n(C(F)F)c4n3)c12] |
| 138 | 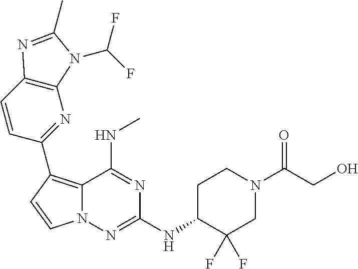 [SMILES: CNc1nc(N[C@@H]2CCN(C(=O)CO)CC2(F)F)nn2ccc(-c3ccc4nc(C)n(C(F)F)c4n3)c12] |
| 139 | 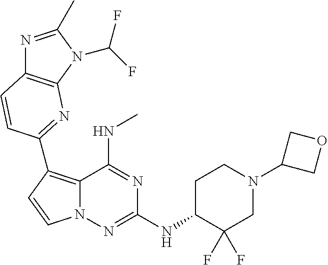 [SMILES: CNc1nc(N[C@@H]2CCN(C3COC3)CC2(F)F)nn2ccc(-c3ccc4nc(C)n(C(F)F)c4n3)c12] |
| 140 | 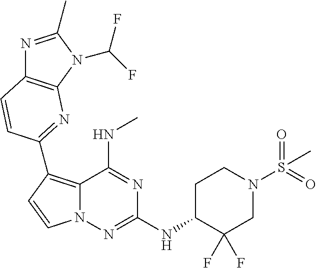 [SMILES: CNc1nc(N[C@@H]2CCN(S(C)(=O)=O)CC2(F)F)nn2ccc(-c3ccc4nc(C)n(C(F)F)c4n3)c12] |
| 141 | 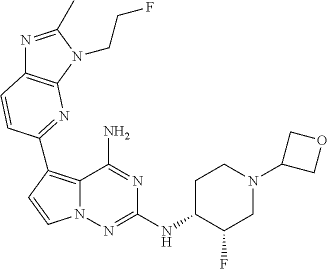 [SMILES: Cc1nc2ccc(-c3ccn4nc(N[C@@H]5CCN(C6COC6)C[C@@H]5F)nc(N)c34)nc2n1CCF] |
| 142 | 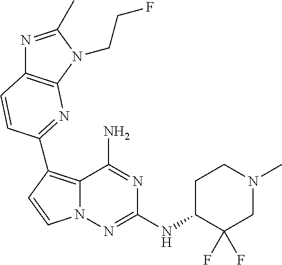 [SMILES: Cc1nc2ccc(-c3ccn4nc(N[C@@H]5CCN(C)CC5(F)F)nc(N)c34)nc2n1CCF] |
| 143 | 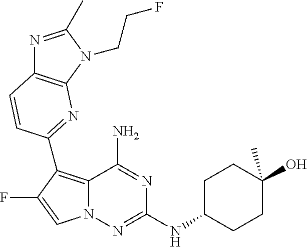 [SMILES: Cc1nc2ccc(-c3c(F)cn4nc(N[C@H]5CC[C@](C)(O)CC5)nc(N)c34)nc2n1CCF] |
| 144 | 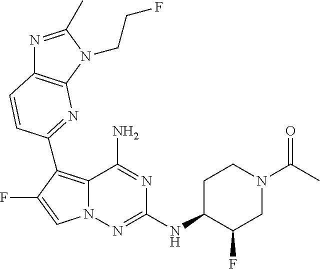 [SMILES: CC(=O)N1CC[C@H](Nc2nc(N)c3c(-c4ccc5nc(C)n(CCF)c5n4)c(F)cn3n2)[C@H](F)C1] |
| 145 | 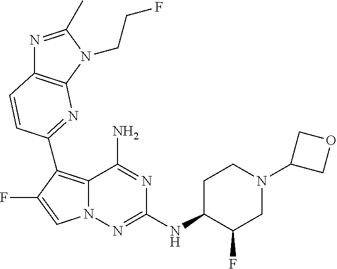 [SMILES: Cc1nc2ccc(-c3c(F)cn4nc(N[C@H]5CCN(C6COC6)C[C@H]5F)nc(N)c34)nc2n1CCF] |
| 146 | 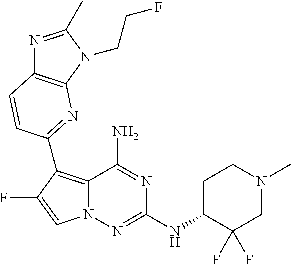 [SMILES: Cc1nc2ccc(-c3c(F)cn4nc(N[C@@H]5CCN(C)CC5(F)F)nc(N)c34)nc2n1CCF] |
| 147 | 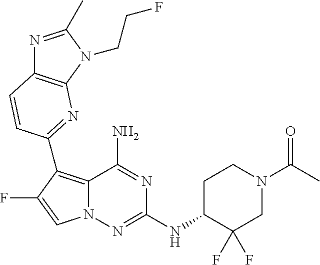 [SMILES: CC(=O)N1CC[C@@H](Nc2nc(N)c3c(-c4ccc5nc(C)n(CCF)c5n4)c(F)cn3n2)C(F)(F)C1] |
| 148 | 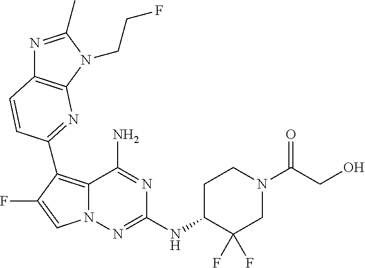 [SMILES: Cc1nc2ccc(-c3c(F)cn4nc(N[C@@H]5CCN(C(=O)CO)CC5(F)F)nc(N)c34)nc2n1CCF] |
| 149 | 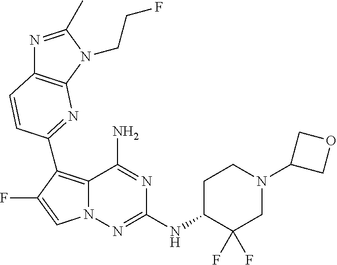 [SMILES: Cc1nc2ccc(-c3c(F)cn4nc(N[C@@H]5CCN(C6COC6)CC5(F)F)nc(N)c34)nc2n1CCF] |
| 150 | 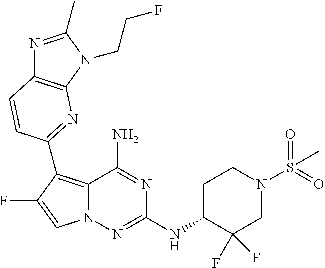 [SMILES: Cc1nc2ccc(-c3c(F)cn4nc(N[C@@H]5CCN(S(C)(=O)=O)CC5(F)F)nc(N)c34)nc2n1CCF] |
| 151 | 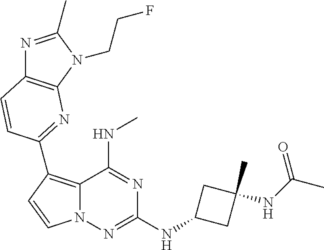 [SMILES: CNc1nc(N[C@H]2C[C@@](C)(NC(C)=O)C2)nn2ccc(-c3ccc4nc(C)n(CCF)c4n3)c12] |
| 152 | 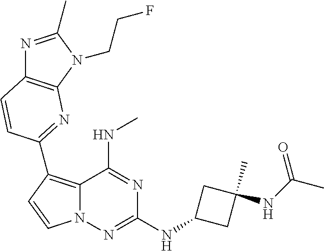 [SMILES: CNc1nc(N[C@H]2C[C@](C)(NC(C)=O)C2)nn2ccc(-c3ccc4nc(C)n(CCF)c4n3)c12] |
| 153 | 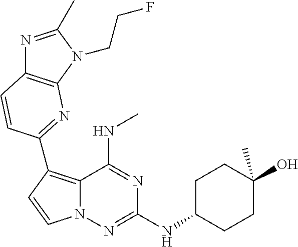 [SMILES: CNc1nc(N[C@H]2CC[C@](C)(O)CC2)nn2ccc(-c3ccc4nc(C)n(CCF)c4n3)c12] |
| 154 | 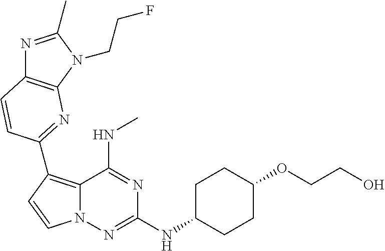 [SMILES: CNc1nc(N[C@H]2CC[C@@H](OCCO)CC2)nn2ccc(-c3ccc4nc(C)n(CCF)c4n3)c12] |
| 155 | 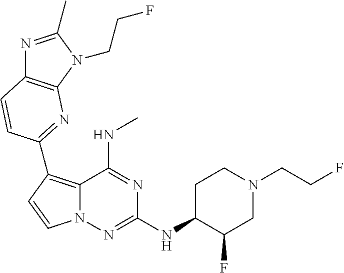 [SMILES: CNc1nc(N[C@H]2CCN(CCF)C[C@H]2F)nn2ccc(-c3ccc4nc(C)n(CCF)c4n3)c12] |
| 156 | 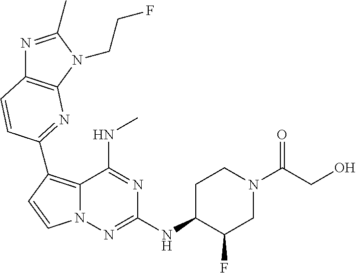 [SMILES: CNc1nc(N[C@H]2CCN(C(=O)CO)C[C@H]2F)nn2ccc(-c3ccc4nc(C)n(CCF)c4n3)c12] |
| 157 | 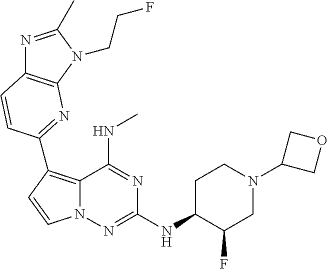 [SMILES: CNc1nc(N[C@H]2CCN(C3COC3)C[C@H]2F)nn2ccc(-c3ccc4nc(C)n(CCF)c4n3)c12] |
| 158 | 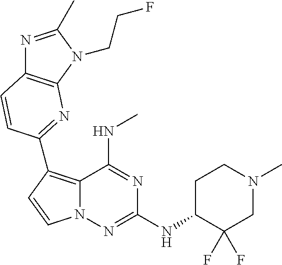 [SMILES: CNc1nc(N[C@@H]2CCN(C)CC2(F)F)nn2ccc(-c3ccc4nc(C)n(CCF)c4n3)c12] |
| 159 | 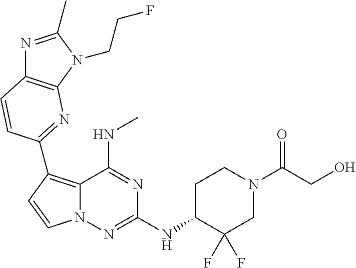 [SMILES: CNc1nc(N[C@@H]2CCN(C(=O)CO)CC2(F)F)nn2ccc(-c3ccc4nc(C)n(CCF)c4n3)c12] |
| 160 | 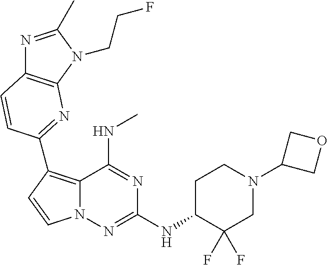 [SMILES: CNc1nc(N[C@@H]2CCN(C3COC3)CC2(F)F)nn2ccc(-c3ccc4nc(C)n(CCF)c4n3)c12] |
| 161 | 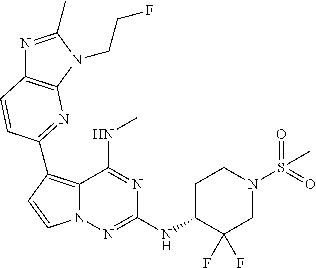 [SMILES: CNc1nc(N[C@@H]2CCN(S(C)(=O)=O)CC2(F)F)nn2ccc(-c3ccc4nc(C)n(CCF)c4n3)c12] |
| 162 | 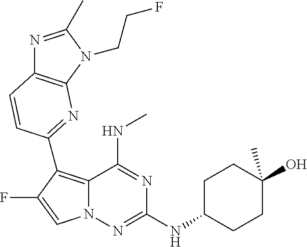 [SMILES: CNc1nc(N[C@H]2CC[C@](C)(O)CC2)nn2cc(F)c(-c3ccc4nc(C)n(CCF)c4n3)c12] |
| 163 | 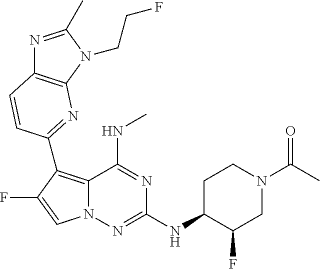 [SMILES: CNc1nc(N[C@H]2CCN(C(C)=O)C[C@H]2F)nn2cc(F)c(-c3ccc4nc(C)n(CCF)c4n3)c12] |
| 164 | 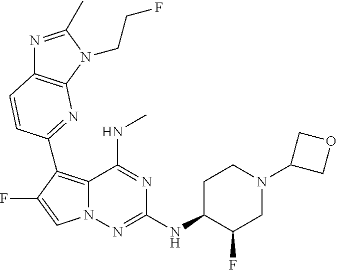 [SMILES: CNc1nc(N[C@H]2CCN(C3COC3)C[C@H]2F)nn2cc(F)c(-c3ccc4nc(C)n(CCF)c4n3)c12] |
| 165 | 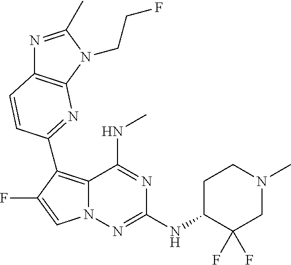 [SMILES: CNc1nc(N[C@@H]2CCN(C)CC2(F)F)nn2cc(F)c(-c3ccc4nc(C)n(CCF)c4n3)c12] |
| 166 | 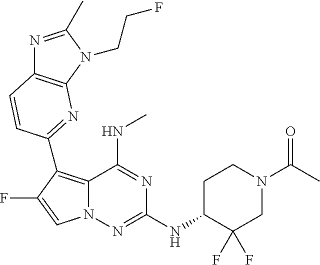 [SMILES: CNc1nc(N[C@@H]2CCN(C(C)=O)CC2(F)F)nn2cc(F)c(-c3ccc4nc(C)n(CCF)c4n3)c12] |
| 167 | 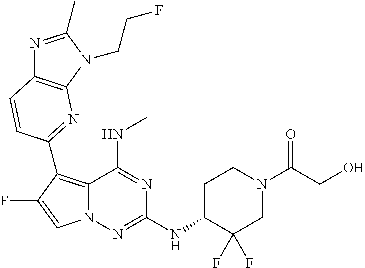 [SMILES: CNc1nc(N[C@@H]2CCN(C(=O)CO)CC2(F)F)nn2cc(F)c(-c3ccc4nc(C)n(CCF)c4n3)c12] |
| 168 | 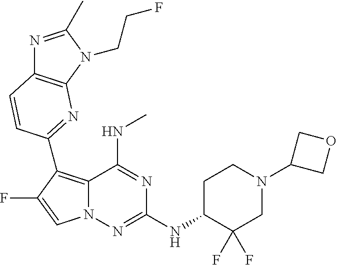 [SMILES: CNc1nc(N[C@@H]2CCN(C3COC3)CC2(F)F)nn2cc(F)c(-c3ccc4nc(C)n(CCF)c4n3)c12] |
| 169 | 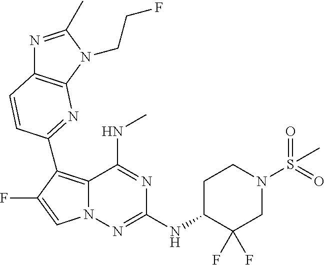 [SMILES: CNc1nc(N[C@@H]2CCN(S(C)(=O)=O)CC2(F)F)nn2cc(F)c(-c3ccc4nc(C)n(CCF)c4n3)c12] |
| 170 | 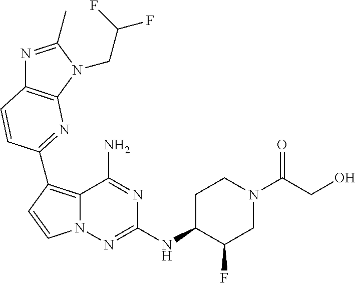 [SMILES: Cc1nc2ccc(-c3ccn4nc(N[C@H]5CCN(C(=O)CO)C[C@H]5F)nc(N)c34)nc2n1CC(F)F] |
| 171 | 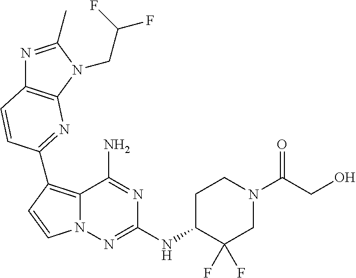 [SMILES: Cc1nc2ccc(-c3ccn4nc(N[C@@H]5CCN(C(=O)CO)CC5(F)F)nc(N)c34)nc2n1CC(F)F] |
| 172 | 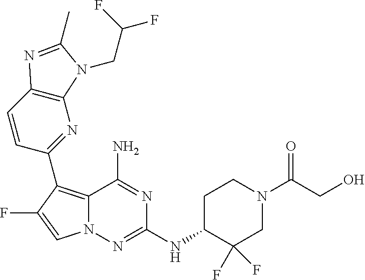 [SMILES: Cc1nc2ccc(-c3c(F)cn4nc(N[C@@H]5CCN(C(=O)CO)CC5(F)F)nc(N)c34)nc2n1CC(F)F] |
| 173 | 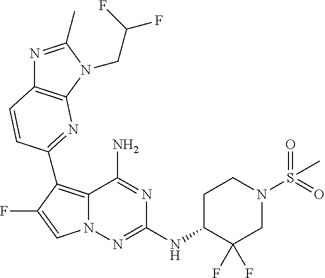 [SMILES: Cc1nc2ccc(-c3c(F)cn4nc(N[C@@H]5CCN(S(C)(=O)=O)CC5(F)F)nc(N)c34)nc2n1CC(F)F] |
| 174 | 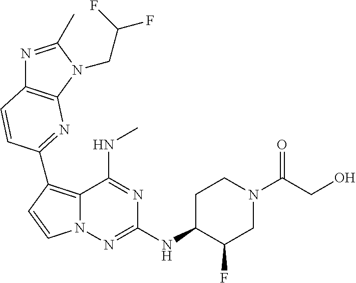 [SMILES: CNc1nc(N[C@H]2CCN(C(=O)CO)C[C@H]2F)nn2ccc(-c3ccc4nc(C)n(CC(F)F)c4n3)c12] |
| 175 | 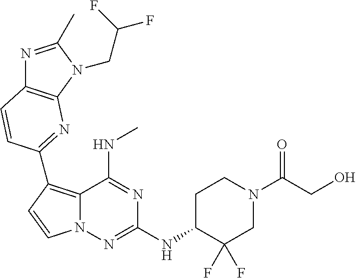 [SMILES: CNc1nc(N[C@@H]2CCN(C(=O)CO)CC2(F)F)nn2ccc(-c3ccc4nc(C)n(CC(F)F)c4n3)c12] |
| 176 | 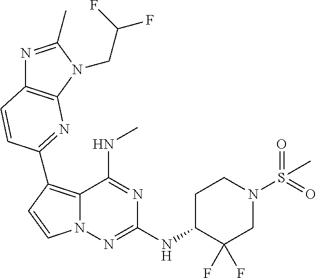 [SMILES: CNc1nc(N[C@@H]2CCN(S(C)(=O)=O)CC2(F)F)nn2ccc(-c3ccc4nc(C)n(CC(F)F)c4n3)c12] |
| 177 | 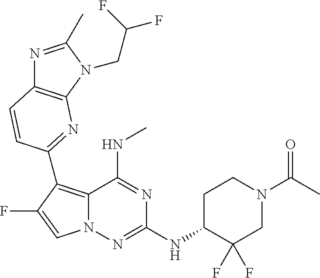 [SMILES: CNc1nc(N[C@@H]2CCN(C(C)=O)CC2(F)F)nn2cc(F)c(-c3ccc4nc(C)n(CC(F)F)c4n3)c12] |
| 178 | 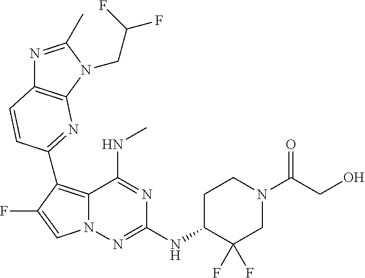 [SMILES: CNc1nc(N[C@@H]2CCN(C(=O)CO)CC2(F)F)nn2cc(F)c(-c3ccc4nc(C)n(CC(F)F)c4n3)c12] |
| 179 | 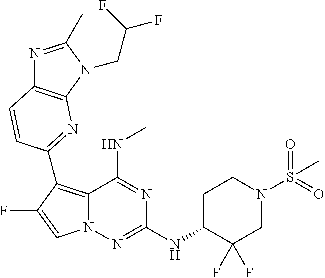 [SMILES: CNc1nc(N[C@@H]2CCN(S(C)(=O)=O)CC2(F)F)nn2cc(F)c(-c3ccc4nc(C)n(CC(F)F)c4n3)c12] |
| 180 | 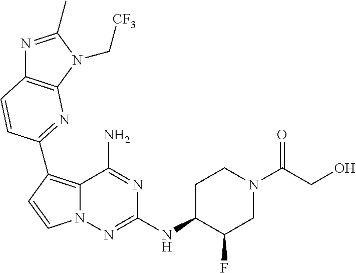 [SMILES: Cc1nc2ccc(-c3ccn4nc(N[C@H]5CCN(C(=O)CO)C[C@H]5F)nc(N)c34)nc2n1CC(F)(F)F] |
| 181 | 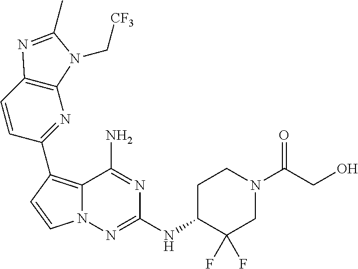 [SMILES: Cc1nc2ccc(-c3ccn4nc(N[C@@H]5CCN(C(=O)CO)CC5(F)F)nc(N)c34)nc2n1CC(F)(F)F] |
| 182 | 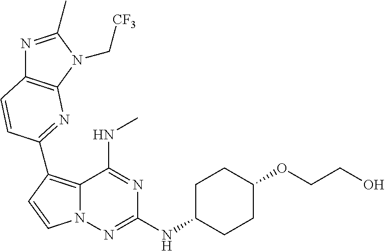 [SMILES: CNc1nc(N[C@H]2CC[C@@H](OCCO)CC2)nn2ccc(-c3ccc4nc(C)n(CC(F)(F)F)c4n3)c12] |
| 183 | 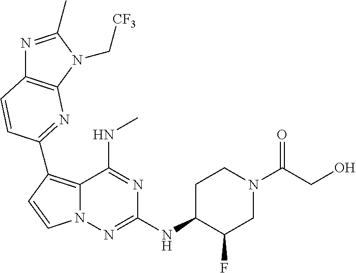 [SMILES: CNc1nc(N[C@H]2CCN(C(=O)CO)C[C@H]2F)nn2ccc(-c3ccc4nc(C)n(CC(F)(F)F)c4n3)c12] |
| 184 | 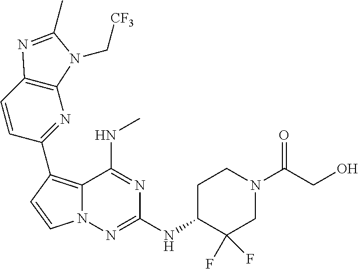 [SMILES: CNc1nc(N[C@@H]2CCN(C(=O)CO)CC2(F)F)nn2ccc(-c3ccc4nc(C)n(CC(F)(F)F)c4n3)c12] |
| 185 | 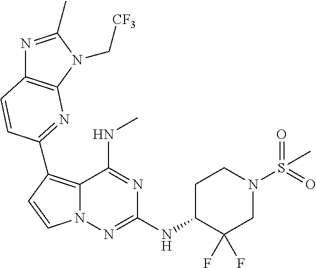 [SMILES: CNc1nc(N[C@@H]2CCN(S(C)(=O)=O)CC2(F)F)nn2ccc(-c3ccc4nc(C)n(CC(F)(F)F)c4n3)c12] |
| 186 | 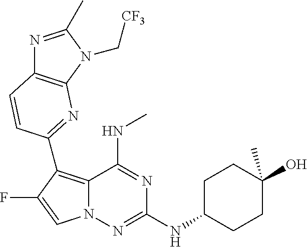 [SMILES: CNc1nc(N[C@H]2CC[C@](C)(O)CC2)nn2cc(F)c(-c3ccc4nc(C)n(CC(F)(F)F)c4n3)c12] |
| 187 | 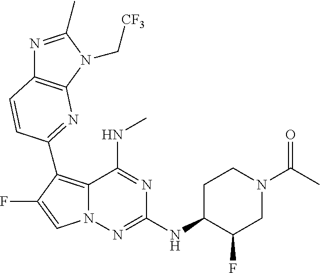 [SMILES: CNc1nc(N[C@H]2CCN(C(C)=O)C[C@H]2F)nn2cc(F)c(-c3ccc4nc(C)n(CC(F)(F)F)c4n3)c12] |
| 188 | 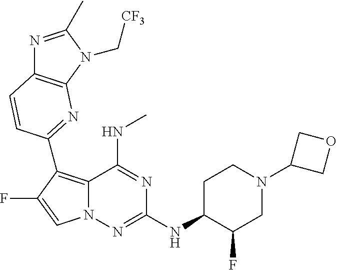 [SMILES: CNc1nc(N[C@H]2CCN(C3COC3)C[C@H]2F)nn2cc(F)c(-c3ccc4nc(C)n(CC(F)(F)F)c4n3)c12] |
| 189 | 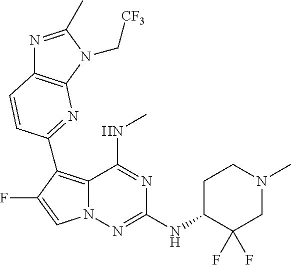 [SMILES: CNc1nc(N[C@@H]2CCN(C)CC2(F)F)nn2cc(F)c(-c3ccc4nc(C)n(CC(F)(F)F)c4n3)c12] |
| 190 | 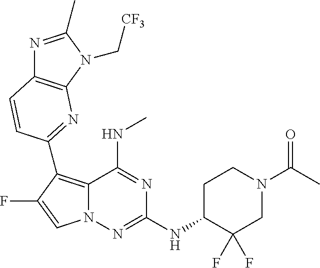 [SMILES: CNc1nc(N[C@@H]2CCN(C(C)=O)CC2(F)F)nn2cc(F)c(-c3ccc4nc(C)n(CC(F)(F)F)c4n3)c12] |
| 191 | 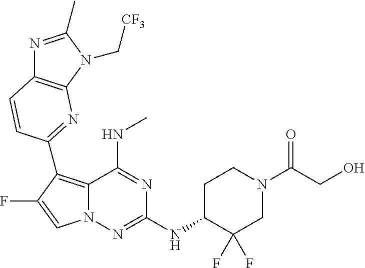 [SMILES: CNc1nc(N[C@@H]2CCN(C(=O)CO)CC2(F)F)nn2cc(F)c(-c3ccc4nc(C)n(CC(F)(F)F)c4n3)c12] |
| 192 | 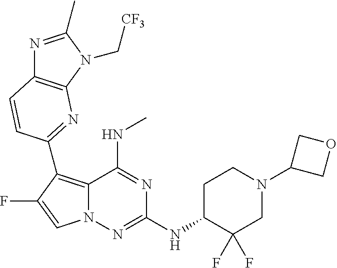 [SMILES: CNc1nc(N[C@@H]2CCN(C3COC3)CC2(F)F)nn2cc(F)c(-c3ccc4nc(C)n(CC(F)(F)F)c4n3)c12] |
| 193 | 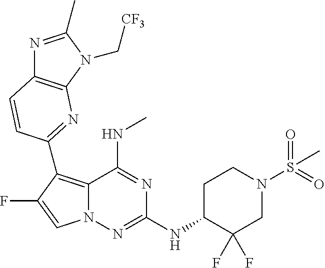 [SMILES: CNc1nc(N[C@@H]2CCN(S(C)(=O)=O)CC2(F)F)nn2cc(F)c(-c3ccc4nc(C)n(CC(F)(F)F)c4n3)c12] |
| 194 | 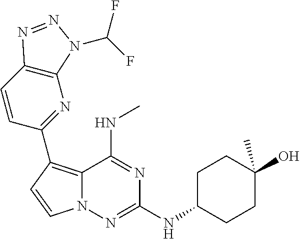 [SMILES: CNc1nc(N[C@H]2CC[C@](C)(O)CC2)nn2ccc(-c3ccc4nnn(C(F)F)c4n3)c12] |
| 195 | 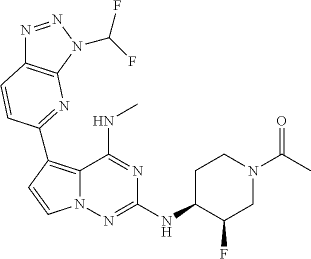 [SMILES: CNc1nc(N[C@H]2CCN(C(C)=O)C[C@H]2F)nn2ccc(-c3ccc4nnn(C(F)F)c4n3)c12] |
| 196 | 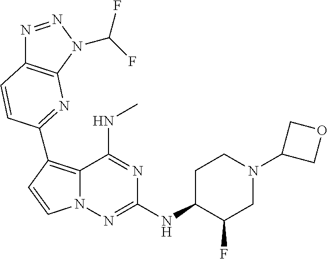 [SMILES: CNc1nc(N[C@H]2CCN(C3COC3)C[C@H]2F)nn2ccc(-c3ccc4nnn(C(F)F)c4n3)c12] |
| 197 | 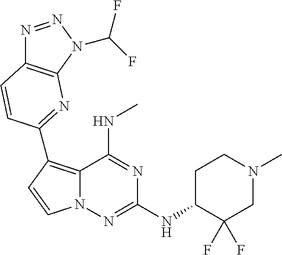 [SMILES: CNc1nc(N[C@@H]2CCN(C)CC2(F)F)nn2ccc(-c3ccc4nnn(C(F)F)c4n3)c12] |
| 198 | 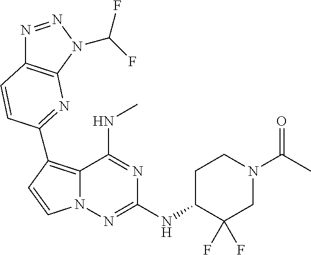 [SMILES: CNc1nc(N[C@@H]2CCN(C(C)=O)CC2(F)F)nn2ccc(-c3ccc4nnn(C(F)F)c4n3)c12] |
| 199 | 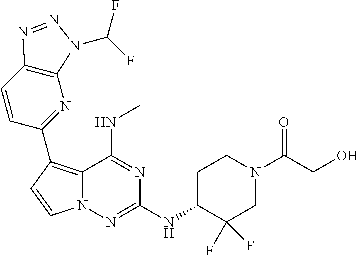 [SMILES: CNc1nc(N[C@@H]2CCN(C(=O)CO)CC2(F)F)nn2ccc(-c3ccc4nnn(C(F)F)c4n3)c12] |
| 200 | 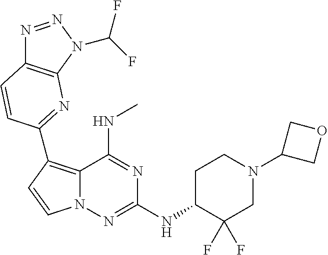 [SMILES: CNc1nc(N[C@@H]2CCN(C3COC3)CC2(F)F)nn2ccc(-c3ccc4nnn(C(F)F)c4n3)c12] |
| 201 | 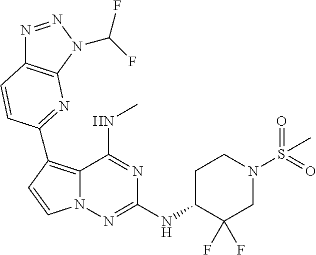 [SMILES: CNc1nc(N[C@@H]2CCN(S(C)(=O)=O)CC2(F)F)nn2ccc(-c3ccc4nnn(C(F)F)c4n3)c12] |
| 202 | 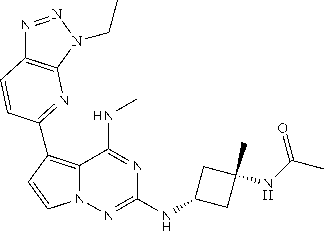 [SMILES: CCn1nnc2ccc(-c3ccn4nc(N[C@H]5C[C@@](C)(NC(C)=O)C5)nc(NC)c34)nc21] |
| 203 | 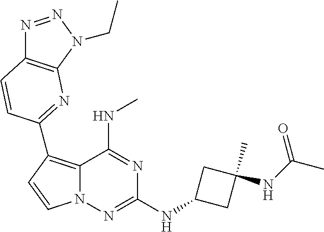 [SMILES: CCn1nnc2ccc(-c3ccn4nc(N[C@H]5C[C@](C)(NC(C)=O)C5)nc(NC)c34)nc21] |
| 204 | 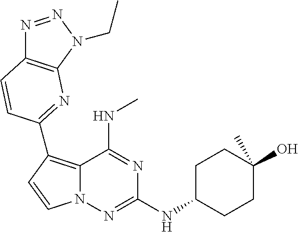 [SMILES: CCn1nnc2ccc(-c3ccn4nc(N[C@H]5CC[C@](C)(O)CC5)nc(NC)c34)nc21] |
| 205 | 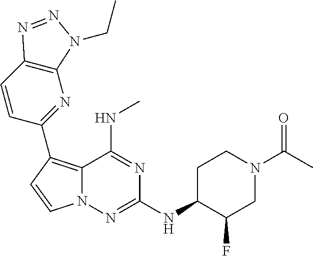 [SMILES: CCn1nnc2ccc(-c3ccn4nc(N[C@H]5CCN(C(C)=O)C[C@H]5F)nc(NC)c34)nc21] |
| 206 | 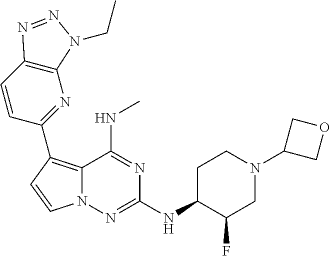 [SMILES: CCn1nnc2ccc(-c3ccn4nc(N[C@H]5CCN(C6COC6)C[C@H]5F)nc(NC)c34)nc21] |
| 207 | 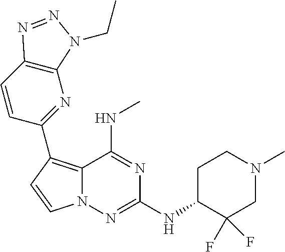 [SMILES: CCn1nnc2ccc(-c3ccn4nc(N[C@@H]5CCN(C)CC5(F)F)nc(NC)c34)nc21] |
| 208 | 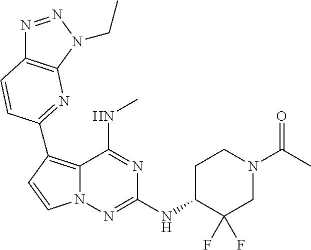 [SMILES: CCn1nnc2ccc(-c3ccn4nc(N[C@@H]5CCN(C(C)=O)CC5(F)F)nc(NC)c34)nc21] |
| 209 | 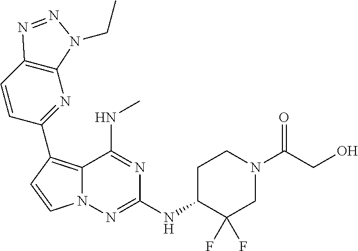 [SMILES: CCn1nnc2ccc(-c3ccn4nc(N[C@@H]5CCN(C(=O)CO)CC5(F)F)nc(NC)c34)nc21] |
| 210 | 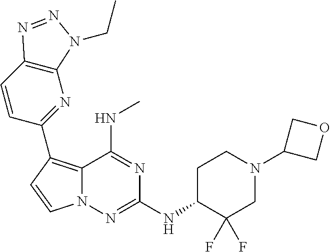 [SMILES: CCn1nnc2ccc(-c3ccn4nc(N[C@@H]5CCN(C6COC6)CC5(F)F)nc(NC)c34)nc21] |
| 211 | 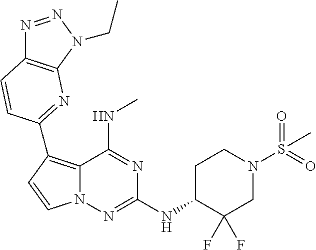 [SMILES: CCn1nnc2ccc(-c3ccn4nc(N[C@@H]5CCN(S(C)(=O)=O)CC5(F)F)nc(NC)c34)nc21] |
| 212 | 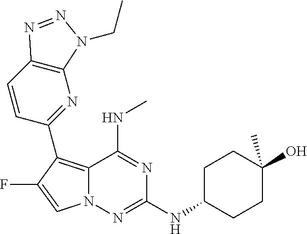 [SMILES: CCn1nnc2ccc(-c3c(F)cn4nc(N[C@H]5CC[C@](C)(O)CC5)nc(NC)c34)nc21] |
| 213 | 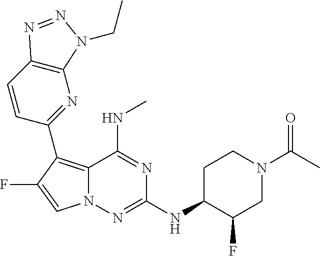 [SMILES: CCn1nnc2ccc(-c3c(F)cn4nc(N[C@H]5CCN(C(C)=O)C[C@H]5F)nc(NC)c34)nc21] |
| 214 | 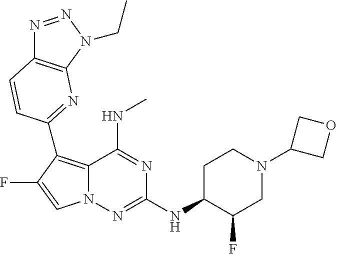 [SMILES: CCn1nnc2ccc(-c3c(F)cn4nc(N[C@H]5CCN(C6COC6)C[C@H]5F)nc(NC)c34)nc21] |
| 215 | 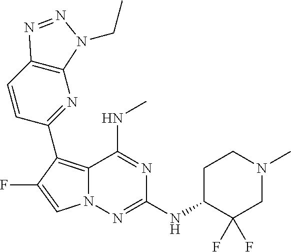 [SMILES: CCn1nnc2ccc(-c3c(F)cn4nc(N[C@@H]5CCN(C)CC5(F)F)nc(NC)c34)nc21] |
| 216 | 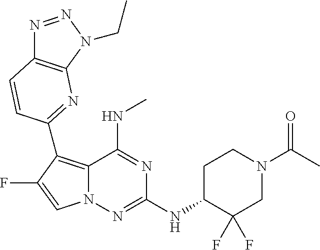 [SMILES: CCn1nnc2ccc(-c3c(F)cn4nc(N[C@@H]5CCN(C(C)=O)CC5(F)F)nc(NC)c34)nc21] |
| 217 | 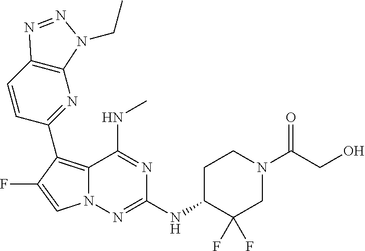 [SMILES: CCn1nnc2ccc(-c3c(F)cn4nc(N[C@@H]5CCN(C(=O)CO)CC5(F)F)nc(NC)c34)nc21] |
| 218 | 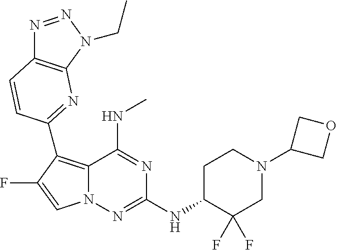 [SMILES: CCn1nnc2ccc(-c3c(F)cn4nc(N[C@@H]5CCN(C6COC6)CC5(F)F)nc(NC)c34)nc21] |
| 219 | 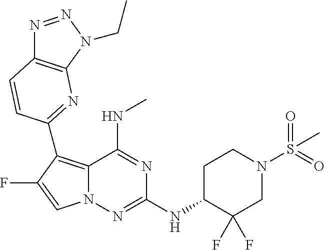 [SMILES: CCn1nnc2ccc(-c3c(F)cn4nc(N[C@@H]5CCN(S(C)(=O)=O)CC5(F)F)nc(NC)c34)nc21] |
| 220 | 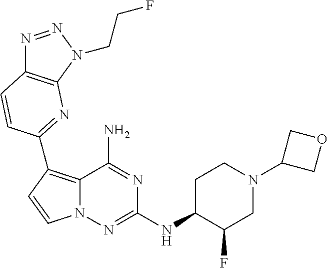 [SMILES: Nc1nc(N[C@H]2CCN(C3COC3)C[C@H]2F)nn2ccc(-c3ccc4nnn(CCF)c4n3)c12] |
| 221 | 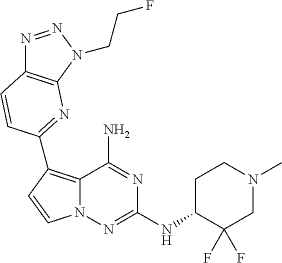 [SMILES: CN1CC[C@@H](Nc2nc(N)c3c(-c4ccc5nnn(CCF)c5n4)ccn3n2)C(F)(F)C1] |
| 222 | 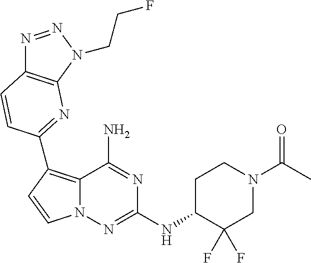 [SMILES: CC(=O)N1CC[C@@H](Nc2nc(N)c3c(-c4ccc5nnn(CCF)c5n4)ccn3n2)C(F)(F)C1] |
| 223 | 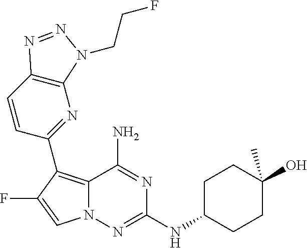 [SMILES: C[C@]1(O)CC[C@H](Nc2nc(N)c3c(-c4ccc5nnn(CCF)c5n4)c(F)cn3n2)CC1] |
| 224 | 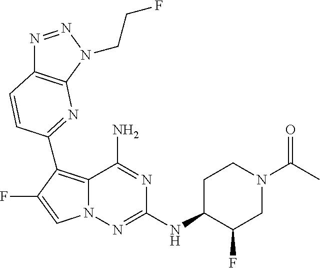 [SMILES: CC(=O)N1CC[C@H](Nc2nc(N)c3c(-c4ccc5nnn(CCF)c5n4)c(F)cn3n2)[C@H](F)C1] |
| 225 | 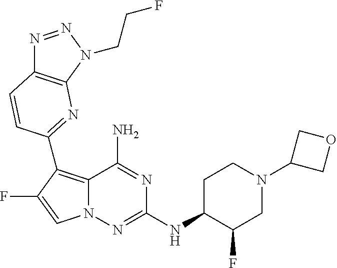 [SMILES: Nc1nc(N[C@H]2CCN(C3COC3)C[C@H]2F)nn2cc(F)c(-c3ccc4nnn(CCF)c4n3)c12] |
| 226 | 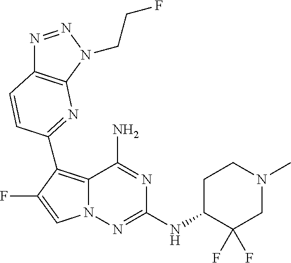 [SMILES: CN1CC[C@@H](Nc2nc(N)c3c(-c4ccc5nnn(CCF)c5n4)c(F)cn3n2)C(F)(F)C1] |
| 227 | 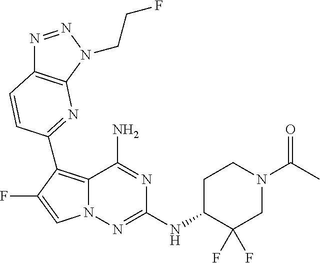 [SMILES: CC(=O)N1CC[C@@H](Nc2nc(N)c3c(-c4ccc5nnn(CCF)c5n4)c(F)cn3n2)C(F)(F)C1] |
| 228 | 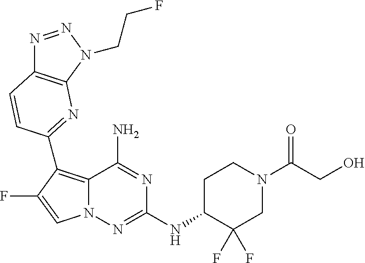 [SMILES: Nc1nc(N[C@@H]2CCN(C(=O)CO)CC2(F)F)nn2cc(F)c(-c3ccc4nnn(CCF)c4n3)c12] |
| 229 | 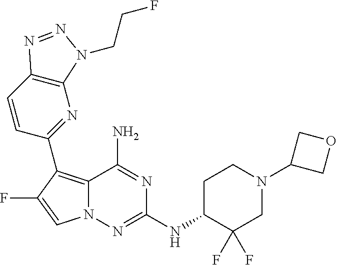 [SMILES: Nc1nc(N[C@@H]2CCN(C3COC3)CC2(F)F)nn2cc(F)c(-c3ccc4nnn(CCF)c4n3)c12] |
| 230 | 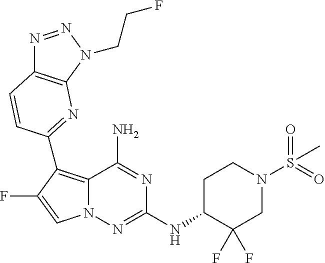 [SMILES: CS(=O)(=O)N1CC[C@@H](Nc2nc(N)c3c(-c4ccc5nnn(CCF)c5n4)c(F)cn3n2)C(F)(F)C1] |
| 231 | 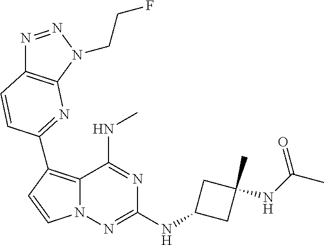 [SMILES: CNc1nc(N[C@H]2C[C@@](C)(NC(C)=O)C2)nn2ccc(-c3ccc4nnn(CCF)c4n3)c12] |
| 232 | 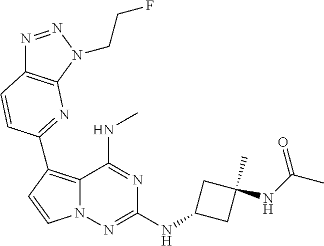 [SMILES: CNc1nc(N[C@H]2C[C@](C)(NC(C)=O)C2)nn2ccc(-c3ccc4nnn(CCF)c4n3)c12] |
| 233 | 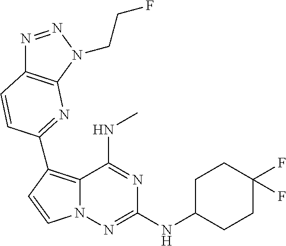 [SMILES: CNc1nc(NC2CCC(F)(F)CC2)nn2ccc(-c3ccc4nnn(CCF)c4n3)c12] |
| 234 | 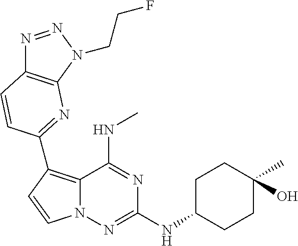 [SMILES: CNc1nc(N[C@H]2CC[C@](C)(O)CC2)nn2ccc(-c3ccc4nnn(CCF)c4n3)c12] |
| 235 | 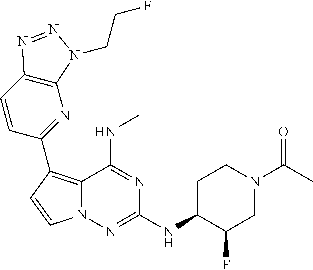 [SMILES: CNc1nc(N[C@H]2CCN(C(C)=O)C[C@H]2F)nn2ccc(-c3ccc4nnn(CCF)c4n3)c12] |
| 236 | 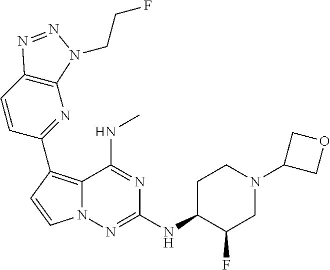 [SMILES: CNc1nc(N[C@H]2CCN(C3COC3)C[C@H]2F)nn2ccc(-c3ccc4nnn(CCF)c4n3)c12] |
| 237 | 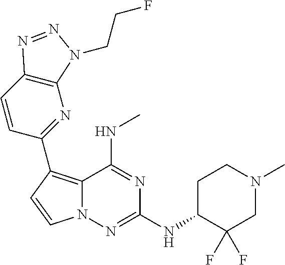 [SMILES: CNc1nc(N[C@@H]2CCN(C)CC2(F)F)nn2ccc(-c3ccc4nnn(CCF)c4n3)c12] |
| 238 | 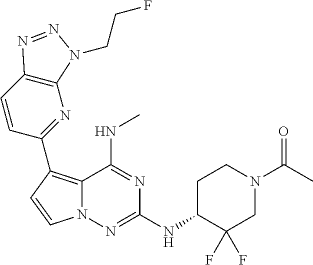 [SMILES: CNc1nc(N[C@@H]2CCN(C(C)=O)CC2(F)F)nn2ccc(-c3ccc4nnn(CCF)c4n3)c12] |
| 239 | 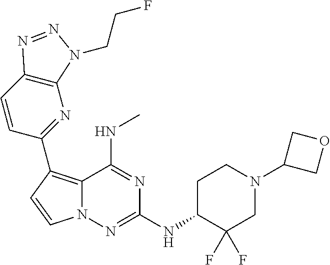 [SMILES: CNc1nc(N[C@@H]2CCN(C3COC3)CC2(F)F)nn2ccc(-c3ccc4nnn(CCF)c4n3)c12] |
| 240 | 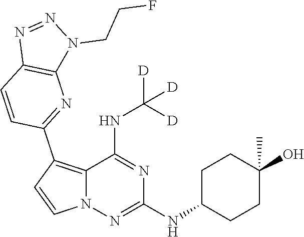 [SMILES: [2H]C([2H])([2H])Nc1nc(N[C@H]2CC[C@](C)(O)CC2)nn2ccc(-c3ccc4nnn(CCF)c4n3)c12] |
| 241 | 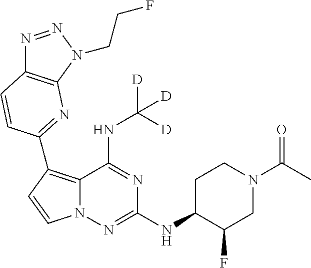 [SMILES: [2H]C([2H])([2H])Nc1nc(N[C@H]2CCN(C(C)=O)C[C@H]2F)nn2ccc(-c3ccc4nnn(CCF)c4n3)c12] |
| 242 | 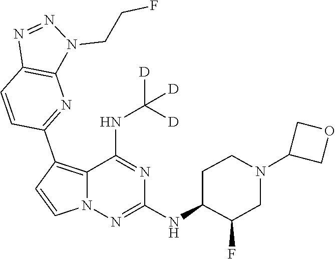 [SMILES: [2H]C([2H])([2H])Nc1nc(N[C@H]2CCN(C3COC3)C[C@H]2F)nn2ccc(-c3ccc4nnn(CCF)c4n3)c12] |
| 243 | 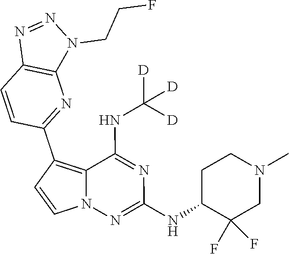 [SMILES: [2H]C([2H])([2H])Nc1nc(N[C@@H]2CCN(C)CC2(F)F)nn2ccc(-c3ccc4nnn(CCF)c4n3)c12] |
| 244 | 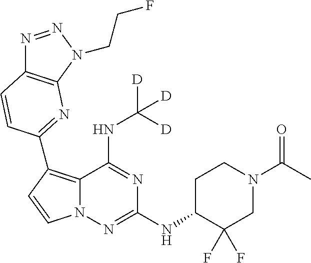 [SMILES: [2H]C([2H])([2H])Nc1nc(N[C@@H]2CCN(C(C)=O)CC2(F)F)nn2ccc(-c3ccc4nnn(CCF)c4n3)c12] |
| 245 | 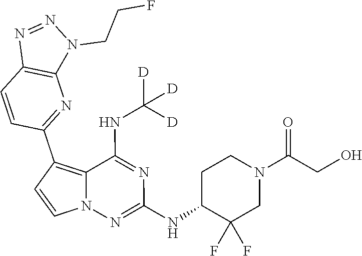 [SMILES: [2H]C([2H])([2H])Nc1nc(N[C@@H]2CCN(C(=O)CO)CC2(F)F)nn2ccc(-c3ccc4nnn(CCF)c4n3)c12] |
| 246 | 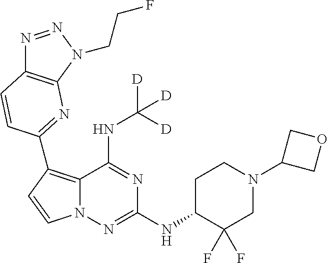 [SMILES: [2H]C([2H])([2H])Nc1nc(N[C@@H]2CCN(C3COC3)CC2(F)F)nn2ccc(-c3ccc4nnn(CCF)c4n3)c12] |
| 247 | 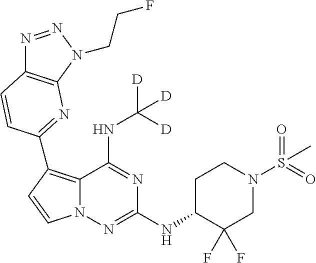 [SMILES: [2H]C([2H])([2H])Nc1nc(N[C@@H]2CCN(S(C)(=O)=O)CC2(F)F)nn2ccc(-c3ccc4nnn(CCF)c4n3)c12] |
| 248 | 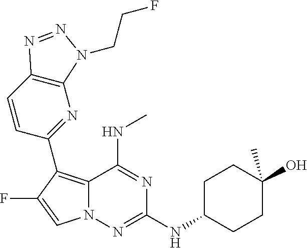 [SMILES: CNc1nc(N[C@H]2CC[C@](C)(O)CC2)nn2cc(F)c(-c3ccc4nnn(CCF)c4n3)c12] |
| 249 | 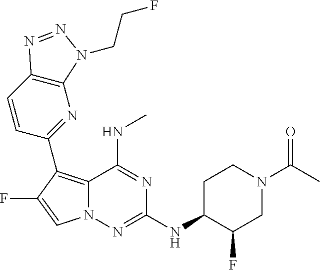 [SMILES: CNc1nc(N[C@H]2CCN(C(C)=O)C[C@H]2F)nn2cc(F)c(-c3ccc4nnn(CCF)c4n3)c12] |
| 250 | 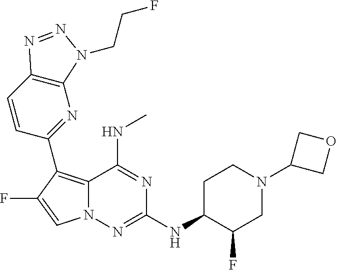 [SMILES: CNc1nc(N[C@H]2CCN(C3COC3)C[C@H]2F)nn2cc(F)c(-c3ccc4nnn(CCF)c4n3)c12] |
| 251 | 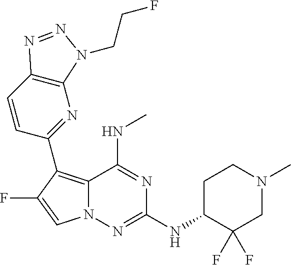 [SMILES: CNc1nc(N[C@@H]2CCN(C)CC2(F)F)nn2cc(F)c(-c3ccc4nnn(CCF)c4n3)c12] |
| 252 | 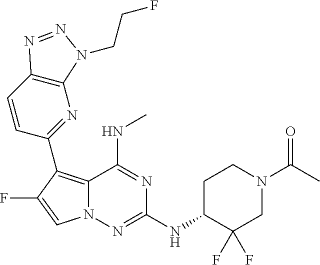 [SMILES: CNc1nc(N[C@@H]2CCN(C(C)=O)CC2(F)F)nn2cc(F)c(-c3ccc4nnn(CCF)c4n3)c12] |
| 253 | 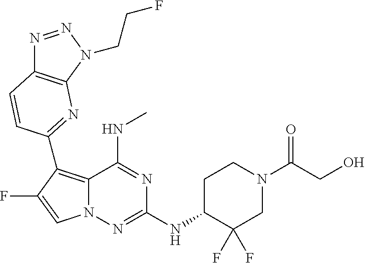 [SMILES: CNc1nc(N[C@@H]2CCN(C(=O)CO)CC2(F)F)nn2cc(F)c(-c3ccc4nnn(CCF)c4n3)c12] |
| 254 | 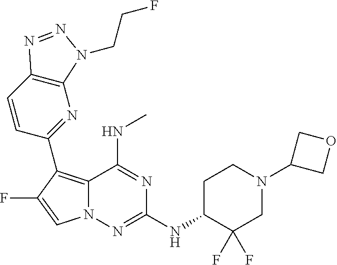 [SMILES: CNc1nc(N[C@@H]2CCN(C3COC3)CC2(F)F)nn2cc(F)c(-c3ccc4nnn(CCF)c4n3)c12] |
| 255 | 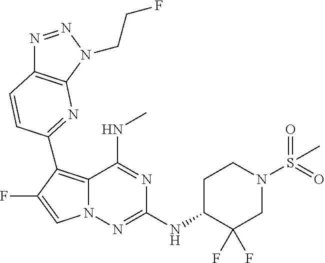 [SMILES: CNc1nc(N[C@@H]2CCN(S(C)(=O)=O)CC2(F)F)nn2cc(F)c(-c3ccc4nnn(CCF)c4n3)c12] |
| 256 | 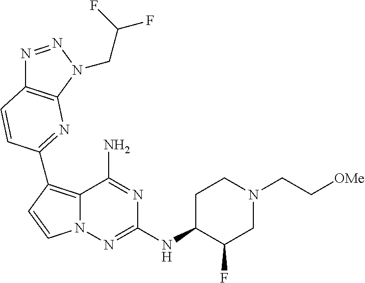 [SMILES: COCCN1CC[C@H](Nc2nc(N)c3c(-c4ccc5nnn(CC(F)F)c5n4)ccn3n2)[C@H](F)C1] |
| 257 | 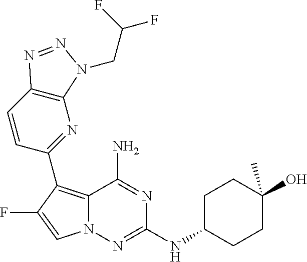 [SMILES: C[C@]1(O)CC[C@H](Nc2nc(N)c3c(-c4ccc5nnn(CC(F)F)c5n4)c(F)cn3n2)CC1] |
| 258 | 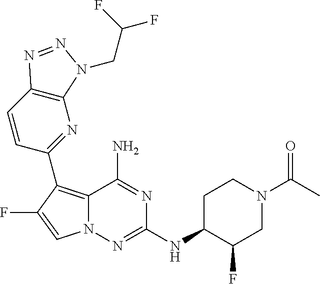 [SMILES: CC(=O)N1CC[C@H](Nc2nc(N)c3c(-c4ccc5nnn(CC(F)F)c5n4)c(F)cn3n2)[C@H](F)C1] |
| 259 | 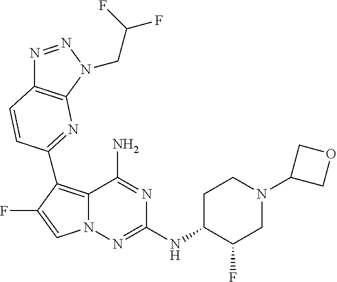 [SMILES: Nc1nc(N[C@@H]2CCN(C3COC3)C[C@@H]2F)nn2cc(F)c(-c3ccc4nnn(CC(F)F)c4n3)c12] |
| 260 | 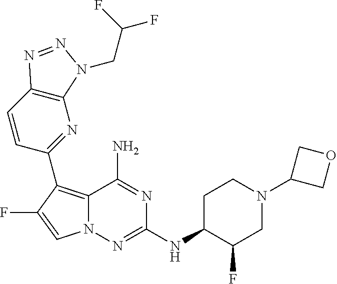 [SMILES: Nc1nc(N[C@H]2CCN(C3COC3)C[C@H]2F)nn2cc(F)c(-c3ccc4nnn(CC(F)F)c4n3)c12] |
| 261 | 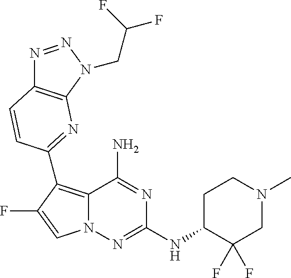 [SMILES: CN1CC[C@@H](Nc2nc(N)c3c(-c4ccc5nnn(CC(F)F)c5n4)c(F)cn3n2)C(F)(F)C1] |
| 262 | 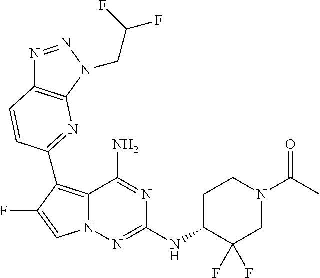 [SMILES: CC(=O)N1CC[C@@H](Nc2nc(N)c3c(-c4ccc5nnn(CC(F)F)c5n4)c(F)cn3n2)C(F)(F)C1] |
| 263 | 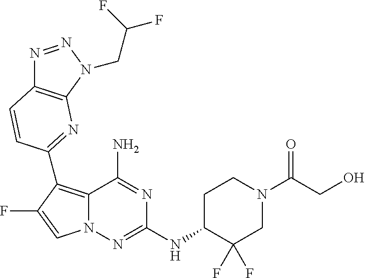 [SMILES: Nc1nc(N[C@@H]2CCN(C(=O)CO)CC2(F)F)nn2cc(F)c(-c3ccc4nnn(CC(F)F)c4n3)c12] |
| 264 | 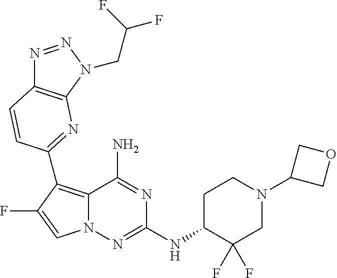 [SMILES: Nc1nc(N[C@@H]2CCN(C3COC3)CC2(F)F)nn2cc(F)c(-c3ccc4nnn(CC(F)F)c4n3)c12] |
| 265 | 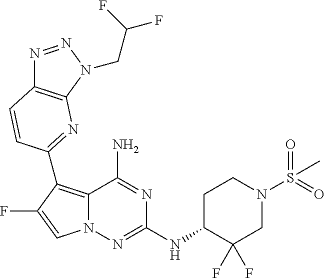 [SMILES: CS(=O)(=O)N1CC[C@@H](Nc2nc(N)c3c(-c4ccc5nnn(CC(F)F)c5n4)c(F)cn3n2)C(F)(F)C1] |
| 266 | 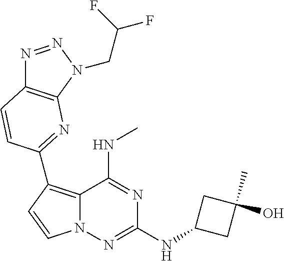 [SMILES: CNc1nc(N[C@H]2C[C@](C)(O)C2)nn2ccc(-c3ccc4nnn(CC(F)F)c4n3)c12] |
| 267 | 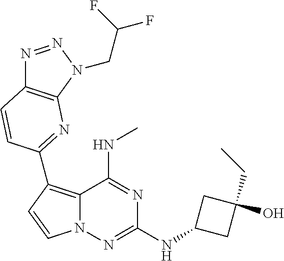 [SMILES: CC[C@]1(O)C[C@H](Nc2nc(NC)c3c(-c4ccc5nnn(CC(F)F)c5n4)ccn3n2)C1] |
| 268 | 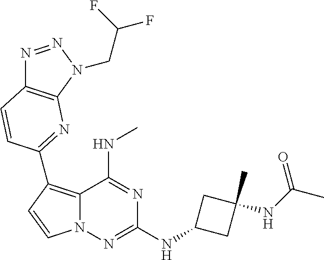 [SMILES: CNc1nc(N[C@H]2C[C@@](C)(NC(C)=O)C2)nn2ccc(-c3ccc4nnn(CC(F)F)c4n3)c12] |
| 269 | 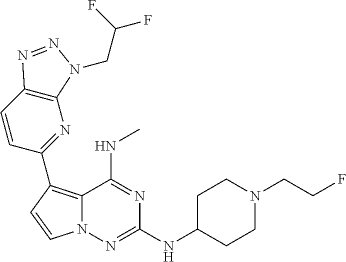 [SMILES: CNc1nc(NC2CCN(CCF)CC2)nn2ccc(-c3ccc4nnn(CC(F)F)c4n3)c12] |
| 270 | 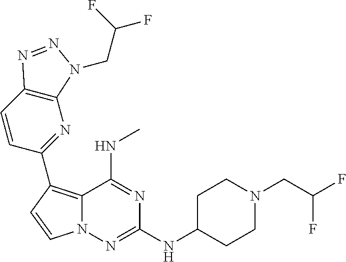 [SMILES: CNc1nc(NC2CCN(CC(F)F)CC2)nn2ccc(-c3ccc4nnn(CC(F)F)c4n3)c12] |
| 271 | 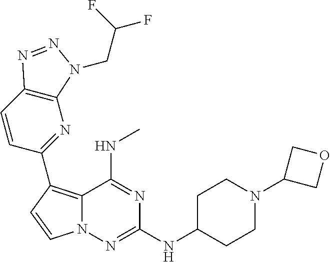 [SMILES: CNc1nc(NC2CCN(C3COC3)CC2)nn2ccc(-c3ccc4nnn(CC(F)F)c4n3)c12] |
| 272 | 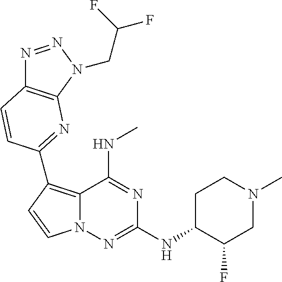 [SMILES: CNc1nc(N[C@@H]2CCN(C)C[C@@H]2F)nn2ccc(-c3ccc4nnn(CC(F)F)c4n3)c12] |
| 273 | 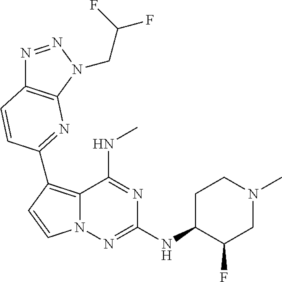 [SMILES: CNc1nc(N[C@H]2CCN(C)C[C@H]2F)nn2ccc(-c3ccc4nnn(CC(F)F)c4n3)c12] |
| 274 | 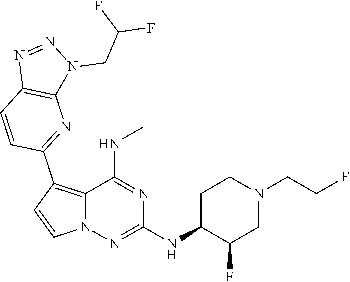 [SMILES: CNc1nc(N[C@H]2CCN(CCF)C[C@H]2F)nn2ccc(-c3ccc4nnn(CC(F)F)c4n3)c12] |
| 275 | 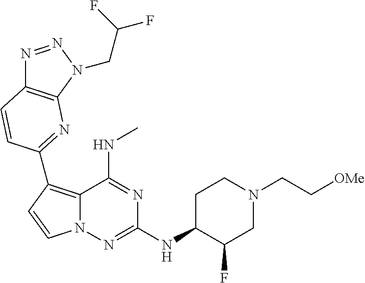 [SMILES: CNc1nc(N[C@H]2CCN(CCOC)C[C@H]2F)nn2ccc(-c3ccc4nnn(CC(F)F)c4n3)c12] |
| 276 | 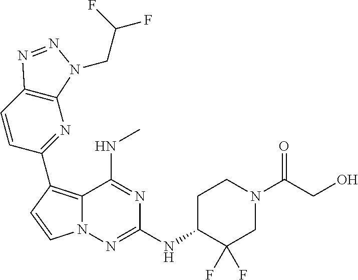 [SMILES: CNc1nc(N[C@@H]2CCN(C(=O)CO)CC2(F)F)nn2ccc(-c3ccc4nnn(CC(F)F)c4n3)c12] |
| 277 | 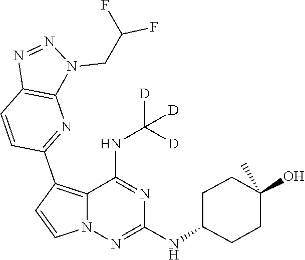 [SMILES: [2H]C([2H])([2H])Nc1nc(N[C@H]2CC[C@](C)(O)CC2)nn2ccc(-c3ccc4nnn(CC(F)F)c4n3)c12] |
| 278 | 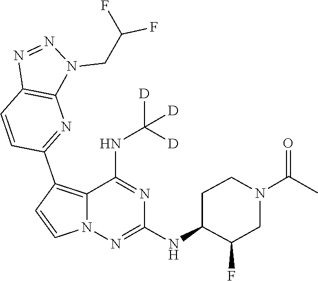 [SMILES: [2H]C([2H])([2H])Nc1nc(N[C@H]2CCN(C(C)=O)C[C@H]2F)nn2ccc(-c3ccc4nnn(CC(F)F)c4n3)c12] |
| 279 | 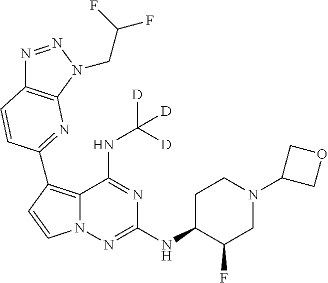 [SMILES: [2H]C([2H])([2H])Nc1nc(N[C@H]2CCN(C3COC3)C[C@H]2F)nn2ccc(-c3ccc4nnn(CC(F)F)c4n3)c12] |
| 280 | 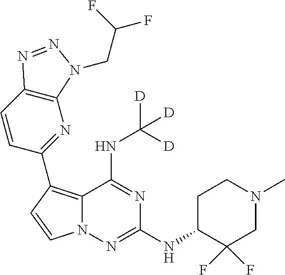 [SMILES: [2H]C([2H])([2H])Nc1nc(N[C@@H]2CCN(C)CC2(F)F)nn2ccc(-c3ccc4nnn(CC(F)F)c4n3)c12] |
| 281 | 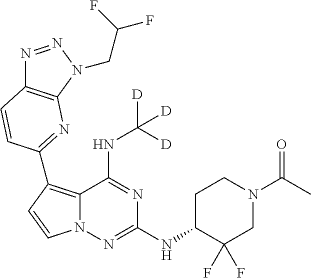 [SMILES: [2H]C([2H])([2H])Nc1nc(N[C@@H]2CCN(C(C)=O)CC2(F)F)nn2ccc(-c3ccc4nnn(CC(F)F)c4n3)c12] |
| 282 | 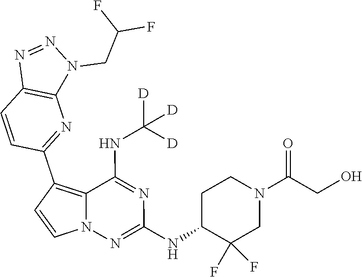 [SMILES: [2H]C([2H])([2H])Nc1nc(N[C@@H]2CCN(C(=O)CO)CC2(F)F)nn2ccc(-c3ccc4nnn(CC(F)F)c4n3)c12] |
| 283 | 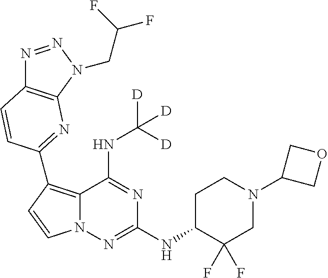 [SMILES: [2H]C([2H])([2H])Nc1nc(N[C@@H]2CCN(C3COC3)CC2(F)F)nn2ccc(-c3ccc4nnn(CC(F)F)c4n3)c12] |
| 284 | 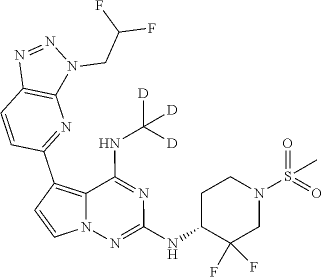 [SMILES: [2H]C([2H])([2H])Nc1nc(N[C@@H]2CCN(S(C)(=O)=O)CC2(F)F)nn2ccc(-c3ccc4nnn(CC(F)F)c4n3)c12] |
| 285 | 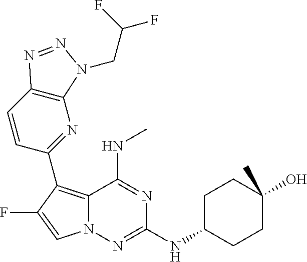 [SMILES: CNc1nc(N[C@H]2CC[C@@](C)(O)CC2)nn2cc(F)c(-c3ccc4nnn(CC(F)F)c4n3)c12] |
| 286 | 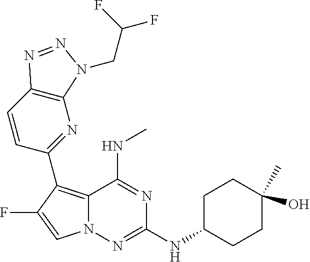 [SMILES: CNc1nc(N[C@H]2CC[C@](C)(O)CC2)nn2cc(F)c(-c3ccc4nnn(CC(F)F)c4n3)c12] |
| 287 | 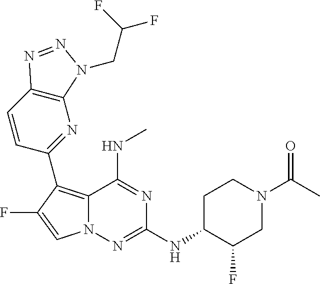 [SMILES: CNc1nc(N[C@@H]2CCN(C(C)=O)C[C@@H]2F)nn2cc(F)c(-c3ccc4nnn(CC(F)F)c4n3)c12] |
| 288 | 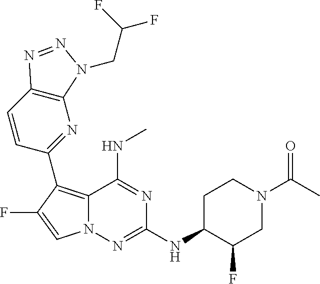 [SMILES: CNc1nc(N[C@H]2CCN(C(C)=O)C[C@H]2F)nn2cc(F)c(-c3ccc4nnn(CC(F)F)c4n3)c12] |
| 289 | 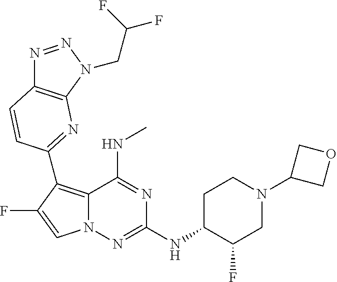 [SMILES: CNc1nc(N[C@@H]2CCN(C3COC3)C[C@@H]2F)nn2cc(F)c(-c3ccc4nnn(CC(F)F)c4n3)c12] |
| 290 | 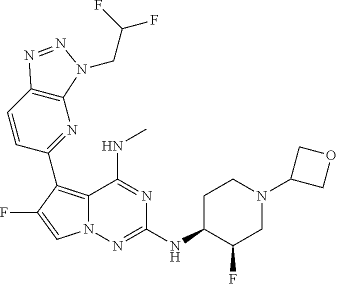 [SMILES: CNc1nc(N[C@H]2CCN(C3COC3)C[C@H]2F)nn2cc(F)c(-c3ccc4nnn(CC(F)F)c4n3)c12] |
| 291 | 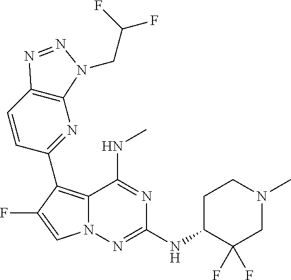 [SMILES: CNc1nc(N[C@@H]2CCN(C)CC2(F)F)nn2cc(F)c(-c3ccc4nnn(CC(F)F)c4n3)c12] |
| 292 | 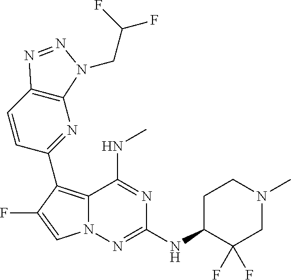 [SMILES: CNc1nc(N[C@H]2CCN(C)CC2(F)F)nn2cc(F)c(-c3ccc4nnn(CC(F)F)c4n3)c12] |
| 293 | 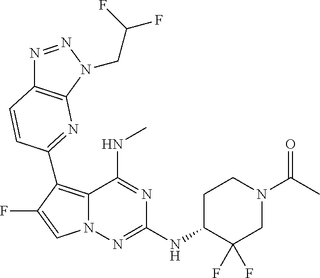 [SMILES: CNc1nc(N[C@@H]2CCN(C(C)=O)CC2(F)F)nn2cc(F)c(-c3ccc4nnn(CC(F)F)c4n3)c12] |
| 294 | 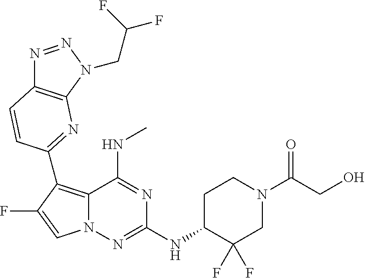 [SMILES: CNc1nc(N[C@@H]2CCN(C(=O)CO)CC2(F)F)nn2cc(F)c(-c3ccc4nnn(CC(F)F)c4n3)c12] |
| 295 | 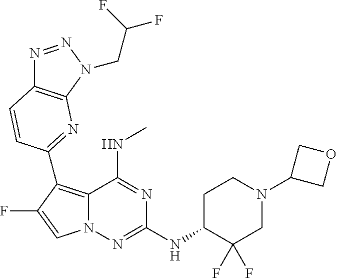 [SMILES: CNc1nc(N[C@@H]2CCN(C3COC3)CC2(F)F)nn2cc(F)c(-c3ccc4nnn(CC(F)F)c4n3)c12] |
| 296 | 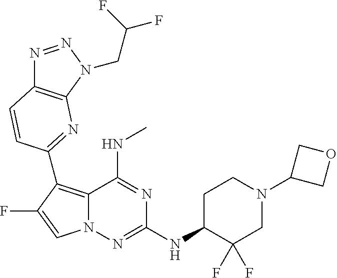 [SMILES: CNc1nc(N[C@H]2CCN(C3COC3)CC2(F)F)nn2cc(F)c(-c3ccc4nnn(CC(F)F)c4n3)c12] |
| 297 | 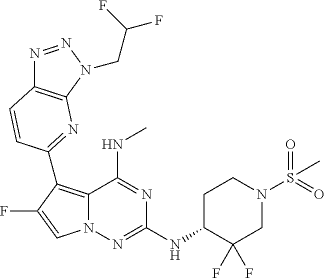 [SMILES: CNc1nc(N[C@@H]2CCN(S(C)(=O)=O)CC2(F)F)nn2cc(F)c(-c3ccc4nnn(CC(F)F)c4n3)c12] |
| 298 | 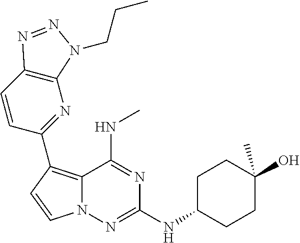 [SMILES: CCCn1nnc2ccc(-c3ccn4nc(N[C@H]5CC[C@](C)(O)CC5)nc(NC)c34)nc21] |
| 299 | 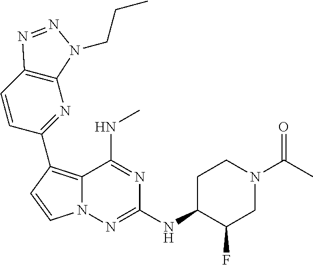 [SMILES: CCCn1nnc2ccc(-c3ccn4nc(N[C@H]5CCN(C(C)=O)C[C@H]5F)nc(NC)c34)nc21] |
| 300 | 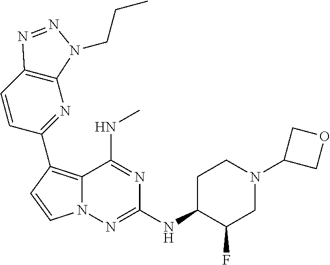 [SMILES: CCCn1nnc2ccc(-c3ccn4nc(N[C@H]5CCN(C6COC6)C[C@H]5F)nc(NC)c34)nc21] |
| 301 | 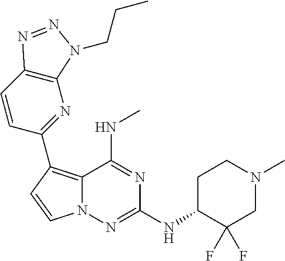 [SMILES: CCCn1nnc2ccc(-c3ccn4nc(N[C@@H]5CCN(C)CC5(F)F)nc(NC)c34)nc21] |
| 302 | 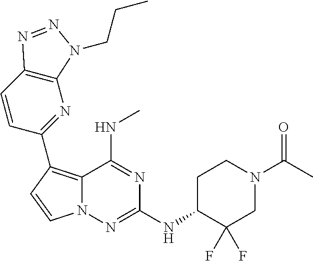 [SMILES: CCCn1nnc2ccc(-c3ccn4nc(N[C@@H]5CCN(C(C)=O)CC5(F)F)nc(NC)c34)nc21] |
| 303 | 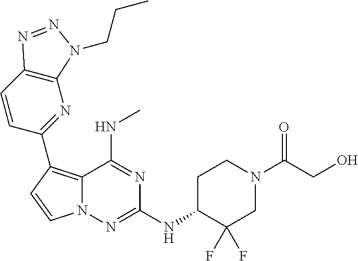 [SMILES: CCCn1nnc2ccc(-c3ccn4nc(N[C@@H]5CCN(C(=O)CO)CC5(F)F)nc(NC)c34)nc21] |
| 304 | 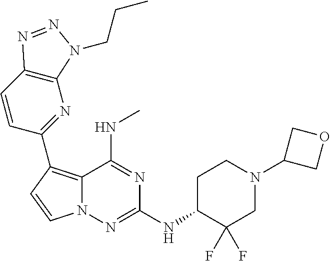 [SMILES: CCCn1nnc2ccc(-c3ccn4nc(N[C@@H]5CCN(C6COC6)CC5(F)F)nc(NC)c34)nc21] |
| 305 | 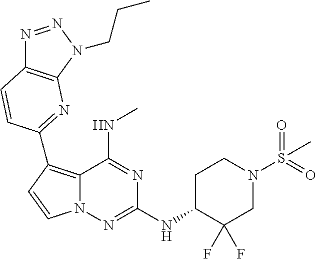 [SMILES: CCCn1nnc2ccc(-c3ccn4nc(N[C@@H]5CCN(S(C)(=O)=O)CC5(F)F)nc(NC)c34)nc21] |
| 306 | 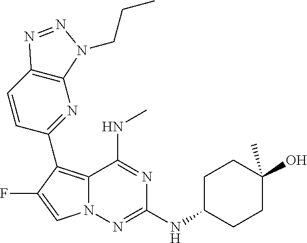 [SMILES: CCCn1nnc2ccc(-c3c(F)cn4nc(N[C@H]5CC[C@](C)(O)CC5)nc(NC)c34)nc21] |
| 307 | 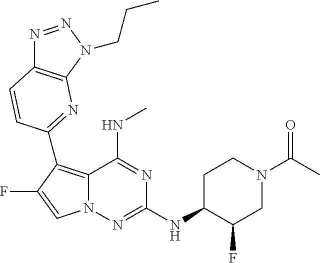 [SMILES: CCCn1nnc2ccc(-c3c(F)cn4nc(N[C@H]5CCN(C(C)=O)C[C@H]5F)nc(NC)c34)nc21] |
| 308 | 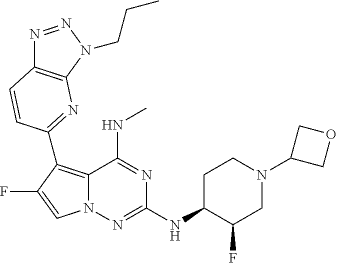 [SMILES: CCCn1nnc2ccc(-c3c(F)cn4nc(N[C@H]5CCN(C6COC6)C[C@H]5F)nc(NC)c34)nc21] |
| 309 | 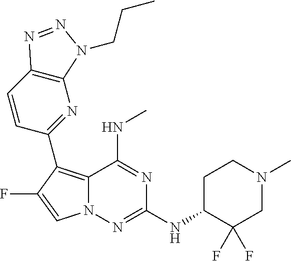 [SMILES: CCCn1nnc2ccc(-c3c(F)cn4nc(N[C@@H]5CCN(C)CC5(F)F)nc(NC)c34)nc21] |
| 310 | 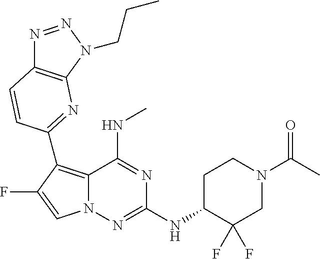 [SMILES: CCCn1nnc2ccc(-c3c(F)cn4nc(N[C@@H]5CCN(C(C)=O)CC5(F)F)nc(NC)c34)nc21] |
| 311 | 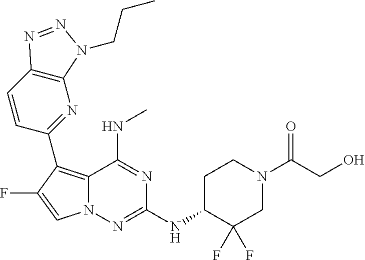 [SMILES: CCCn1nnc2ccc(-c3c(F)cn4nc(N[C@@H]5CCN(C(=O)CO)CC5(F)F)nc(NC)c34)nc21] |
| 312 | 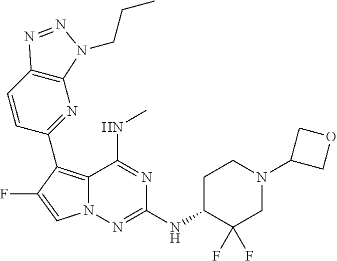 [SMILES: CCCn1nnc2ccc(-c3c(F)cn4nc(N[C@@H]5CCN(C6COC6)CC5(F)F)nc(NC)c34)nc21] |
| 313 | 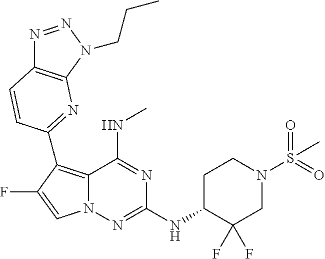 [SMILES: CCCn1nnc2ccc(-c3c(F)cn4nc(N[C@@H]5CCN(S(C)(=O)=O)CC5(F)F)nc(NC)c34)nc21] |
| 314 | 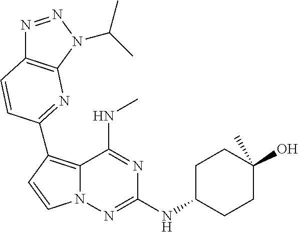 [SMILES: CNc1nc(N[C@H]2CC[C@](C)(O)CC2)nn2ccc(-c3ccc4nnn(C(C)C)c4n3)c12] |
| 315 | 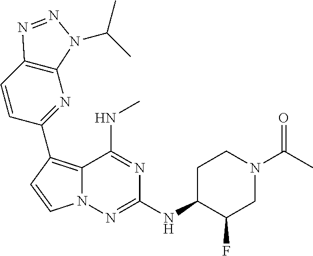 [SMILES: CNc1nc(N[C@H]2CCN(C(C)=O)C[C@H]2F)nn2ccc(-c3ccc4nnn(C(C)C)c4n3)c12] |
| 316 | 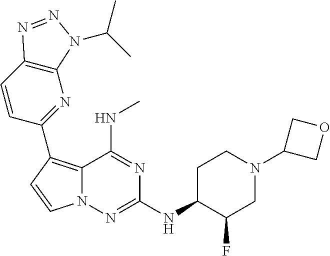 [SMILES: CNc1nc(N[C@H]2CCN(C3COC3)C[C@H]2F)nn2ccc(-c3ccc4nnn(C(C)C)c4n3)c12] |
| 317 | 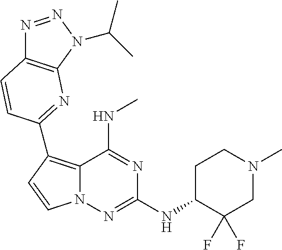 [SMILES: CNc1nc(N[C@@H]2CCN(C)CC2(F)F)nn2ccc(-c3ccc4nnn(C(C)C)c4n3)c12] |
| 318 | 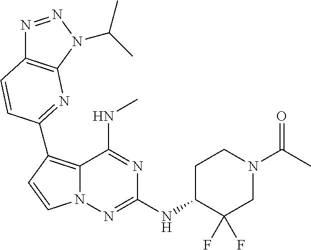 [SMILES: CNc1nc(N[C@@H]2CCN(C(C)=O)CC2(F)F)nn2ccc(-c3ccc4nnn(C(C)C)c4n3)c12] |
| 319 | 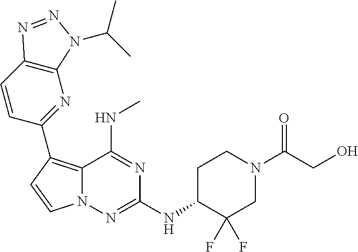 [SMILES: CNc1nc(N[C@@H]2CCN(C(=O)CO)CC2(F)F)nn2ccc(-c3ccc4nnn(C(C)C)c4n3)c12] |
| 320 | 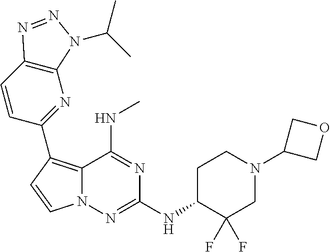 [SMILES: CNc1nc(N[C@@H]2CCN(C3COC3)CC2(F)F)nn2ccc(-c3ccc4nnn(C(C)C)c4n3)c12] |
| 321 | 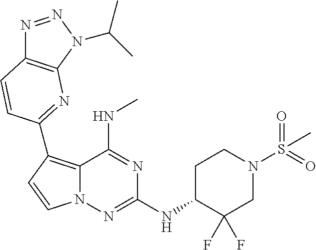 [SMILES: CNc1nc(N[C@@H]2CCN(S(C)(=O)=O)CC2(F)F)nn2ccc(-c3ccc4nnn(C(C)C)c4n3)c12] |
| 322 | 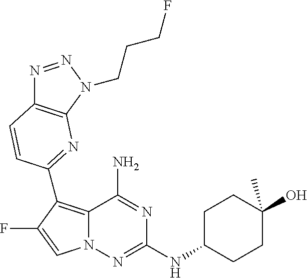 [SMILES: C[C@]1(O)CC[C@H](Nc2nc(N)c3c(-c4ccc5nnn(CCCF)c5n4)c(F)cn3n2)CC1] |
| 323 | 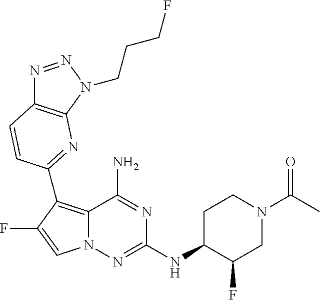 [SMILES: CC(=O)N1CC[C@H](Nc2nc(N)c3c(-c4ccc5nnn(CCCF)c5n4)c(F)cn3n2)[C@H](F)C1] |
| 324 | 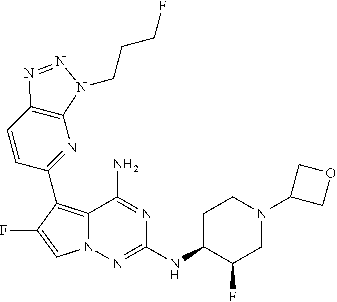 [SMILES: Nc1nc(N[C@H]2CCN(C3COC3)C[C@H]2F)nn2cc(F)c(-c3ccc4nnn(CCCF)c4n3)c12] |
| 325 | 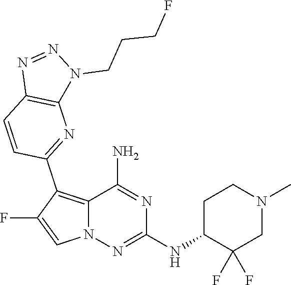 [SMILES: CN1CC[C@@H](Nc2nc(N)c3c(-c4ccc5nnn(CCCF)c5n4)c(F)cn3n2)C(F)(F)C1] |
| 326 | 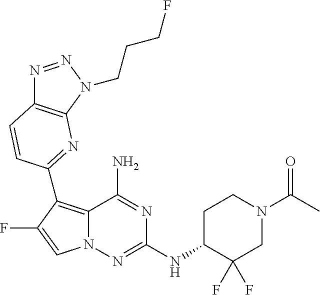 [SMILES: CC(=O)N1CC[C@@H](Nc2nc(N)c3c(-c4ccc5nnn(CCCF)c5n4)c(F)cn3n2)C(F)(F)C1] |
| 327 | 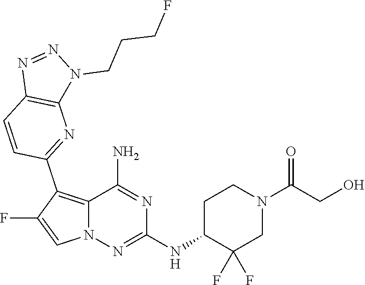 [SMILES: Nc1nc(N[C@@H]2CCN(C(=O)CO)CC2(F)F)nn2cc(F)c(-c3ccc4nnn(CCCF)c4n3)c12] |
| 328 | 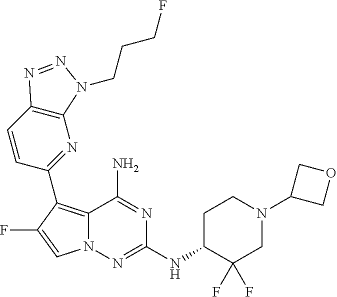 [SMILES: Nc1nc(N[C@@H]2CCN(C3COC3)CC2(F)F)nn2cc(F)c(-c3ccc4nnn(CCCF)c4n3)c12] |
| 329 | 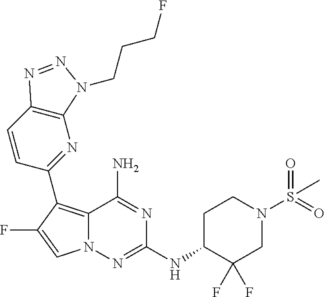 [SMILES: CS(=O)(=O)N1CC[C@@H](Nc2nc(N)c3c(-c4ccc5nnn(CCCF)c5n4)c(F)cn3n2)C(F)(F)C1] |
| 330 | 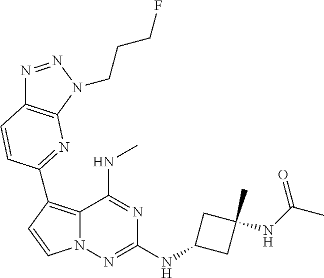 [SMILES: CNc1nc(N[C@H]2C[C@@](C)(NC(C)=O)C2)nn2ccc(-c3ccc4nnn(CCCF)c4n3)c12] |
| 331 | 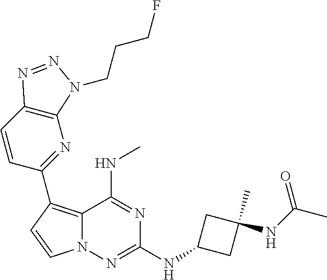 [SMILES: CNc1nc(N[C@H]2C[C@](C)(NC(C)=O)C2)nn2ccc(-c3ccc4nnn(CCCF)c4n3)c12] |
| 332 | 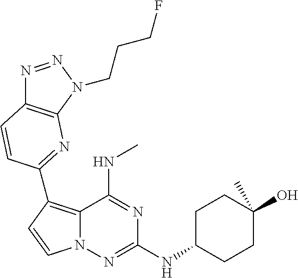 [SMILES: CNc1nc(N[C@H]2CC[C@](C)(O)CC2)nn2ccc(-c3ccc4nnn(CCCF)c4n3)c12] |
| 333 | 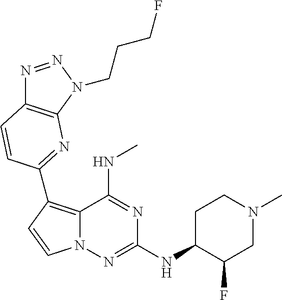 [SMILES: CNc1nc(N[C@H]2CCN(C)C[C@H]2F)nn2ccc(-c3ccc4nnn(CCCF)c4n3)c12] |
| 334 | 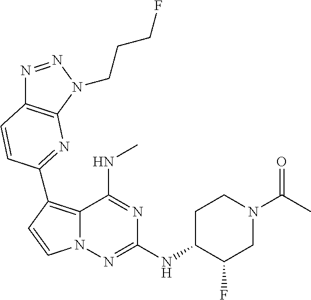 [SMILES: CNc1nc(N[C@@H]2CCN(C(C)=O)C[C@@H]2F)nn2ccc(-c3ccc4nnn(CCCF)c4n3)c12] |
| 335 | 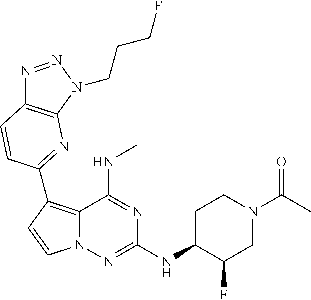 [SMILES: CNc1nc(N[C@H]2CCN(C(C)=O)C[C@H]2F)nn2ccc(-c3ccc4nnn(CCCF)c4n3)c12] |
| 336 | 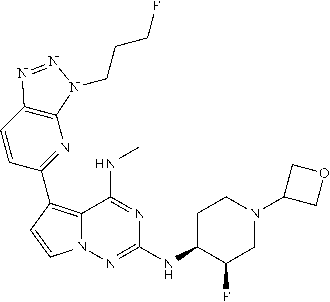 [SMILES: CNc1nc(N[C@H]2CCN(C3COC3)C[C@H]2F)nn2ccc(-c3ccc4nnn(CCCF)c4n3)c12] |
| 337 | 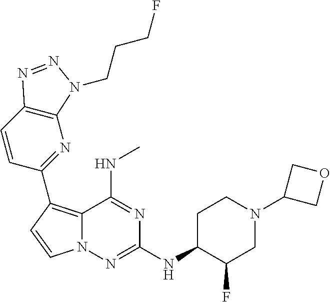 [SMILES: CNc1nc(N[C@H]2CCN(C3COC3)C[C@H]2F)nn2ccc(-c3ccc4nnn(CCCF)c4n3)c12] |
| 338 | 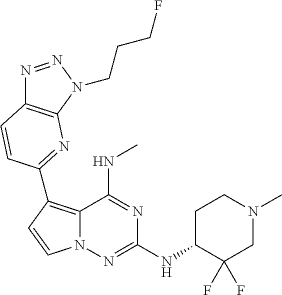 [SMILES: CNc1nc(N[C@@H]2CCN(C)CC2(F)F)nn2ccc(-c3ccc4nnn(CCCF)c4n3)c12] |
| 339 | 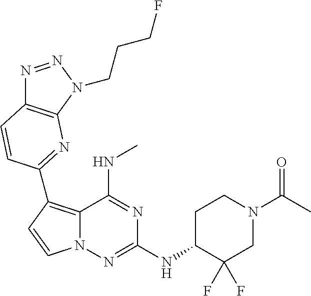 [SMILES: CNc1nc(N[C@@H]2CCN(C(C)=O)CC2(F)F)nn2ccc(-c3ccc4nnn(CCCF)c4n3)c12] |
| 340 | 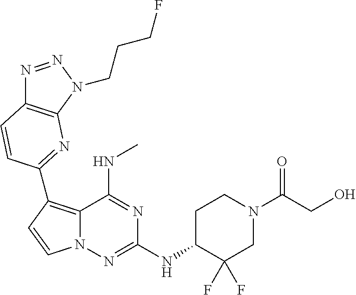 [SMILES: CNc1nc(N[C@@H]2CCN(C(=O)CO)CC2(F)F)nn2ccc(-c3ccc4nnn(CCCF)c4n3)c12] |
| 341 | 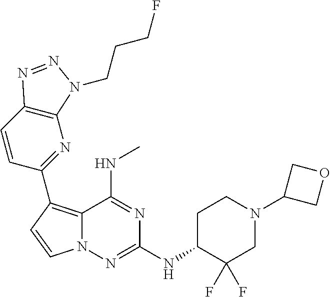 [SMILES: CNc1nc(N[C@@H]2CCN(C3COC3)CC2(F)F)nn2ccc(-c3ccc4nnn(CCCF)c4n3)c12] |
| 342 | 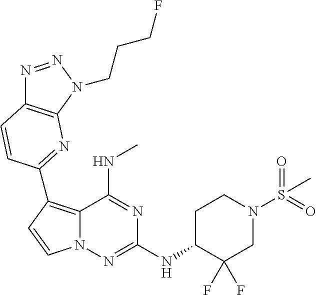 [SMILES: CNc1nc(N[C@@H]2CCN(S(C)(=O)=O)CC2(F)F)nn2ccc(-c3ccc4nnn(CCCF)c4n3)c12] |
| 343 | 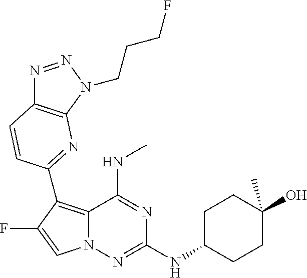 [SMILES: CNc1nc(N[C@H]2CC[C@](C)(O)CC2)nn2cc(F)c(-c3ccc4nnn(CCCF)c4n3)c12] |
| 344 | 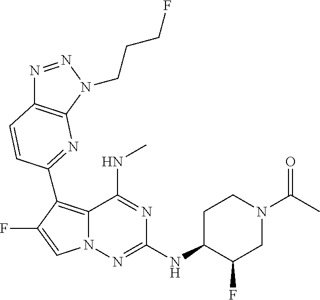 [SMILES: CNc1nc(N[C@H]2CCN(C(C)=O)C[C@H]2F)nn2cc(F)c(-c3ccc4nnn(CCCF)c4n3)c12] |
| 345 | 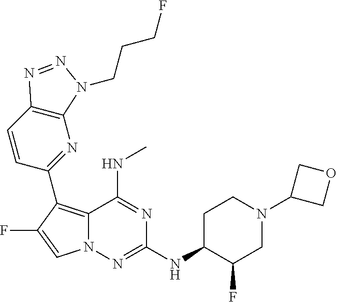 [SMILES: CNc1nc(N[C@H]2CCN(C3COC3)C[C@H]2F)nn2cc(F)c(-c3ccc4nnn(CCCF)c4n3)c12] |
| 346 | 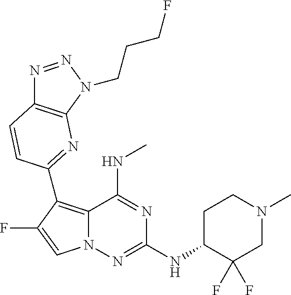 [SMILES: CNc1nc(N[C@@H]2CCN(C)CC2(F)F)nn2cc(F)c(-c3ccc4nnn(CCCF)c4n3)c12] |
| 347 | 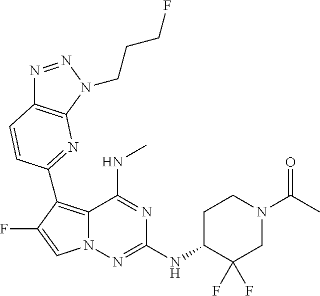 [SMILES: CNc1nc(N[C@@H]2CCN(C(C)=O)CC2(F)F)nn2cc(F)c(-c3ccc4nnn(CCCF)c4n3)c12] |
| 348 | 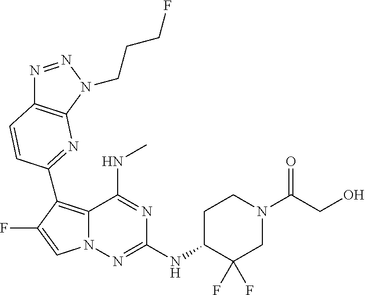 [SMILES: CNc1nc(N[C@@H]2CCN(C(=O)CO)CC2(F)F)nn2cc(F)c(-c3ccc4nnn(CCCF)c4n3)c12] |
| 349 | 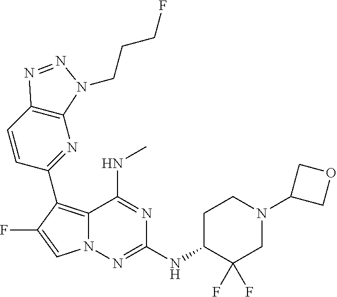 [SMILES: CNc1nc(N[C@@H]2CCN(C3COC3)CC2(F)F)nn2cc(F)c(-c3ccc4nnn(CCCF)c4n3)c12] |
| 350 | 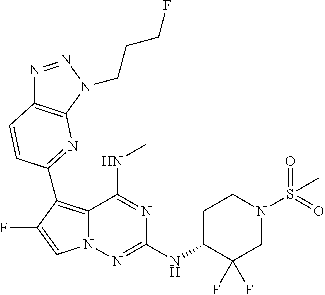 [SMILES: CNc1nc(N[C@@H]2CCN(S(C)(=O)=O)CC2(F)F)nn2cc(F)c(-c3ccc4nnn(CCCF)c4n3)c12] |
| 351 | 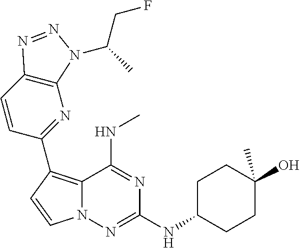 [SMILES: CNc1nc(N[C@H]2CC[C@](C)(O)CC2)nn2ccc(-c3ccc4nnn([C@@H](C)CF)c4n3)c12] |
| 352 | 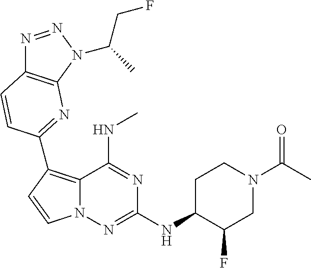 [SMILES: CNc1nc(N[C@H]2CCN(C(C)=O)C[C@H]2F)nn2ccc(-c3ccc4nnn([C@@H](C)CF)c4n3)c12] |
| 353 | 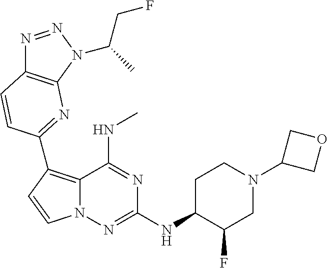 [SMILES: CNc1nc(N[C@H]2CCN(C3COC3)C[C@H]2F)nn2ccc(-c3ccc4nnn([C@@H](C)CF)c4n3)c12] |
| 354 | 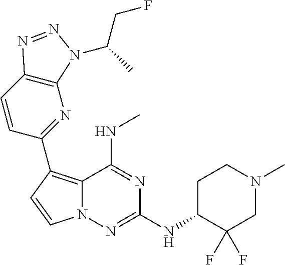 [SMILES: CNc1nc(N[C@@H]2CCN(C)CC2(F)F)nn2ccc(-c3ccc4nnn([C@@H](C)CF)c4n3)c12] |
| 355 | 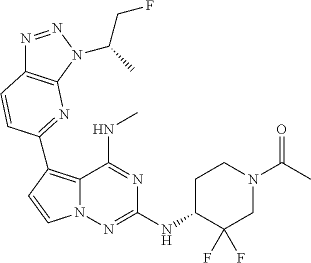 [SMILES: CNc1nc(N[C@@H]2CCN(C(C)=O)CC2(F)F)nn2ccc(-c3ccc4nnn([C@@H](C)CF)c4n3)c12] |
| 356 | 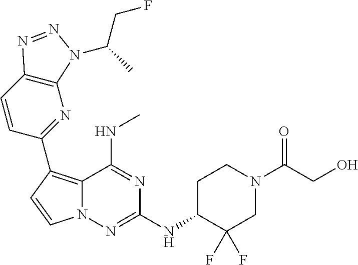 [SMILES: CNc1nc(N[C@@H]2CCN(C(=O)CO)CC2(F)F)nn2ccc(-c3ccc4nnn([C@@H](C)CF)c4n3)c12] |
| 357 | 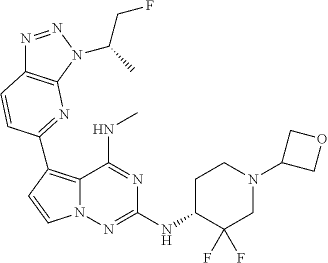 [SMILES: CNc1nc(N[C@@H]2CCN(C3COC3)CC2(F)F)nn2ccc(-c3ccc4nnn([C@@H](C)CF)c4n3)c12] |
| 358 | 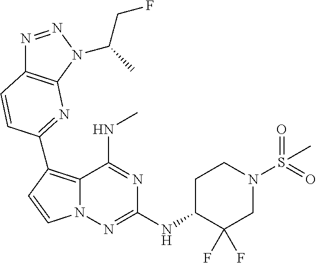 [SMILES: CNc1nc(N[C@@H]2CCN(S(C)(=O)=O)CC2(F)F)nn2ccc(-c3ccc4nnn([C@@H](C)CF)c4n3)c12] |
| 359 | 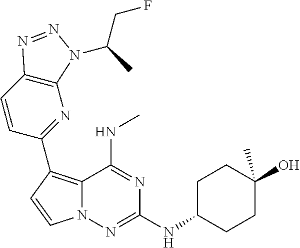 [SMILES: CNc1nc(N[C@H]2CC[C@](C)(O)CC2)nn2ccc(-c3ccc4nnn([C@H](C)CF)c4n3)c12] |
| 360 | 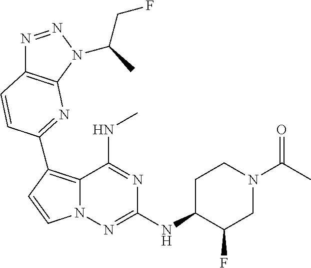 [SMILES: CNc1nc(N[C@H]2CCN(C(C)=O)C[C@H]2F)nn2ccc(-c3ccc4nnn([C@H](C)CF)c4n3)c12] |
| 361 | 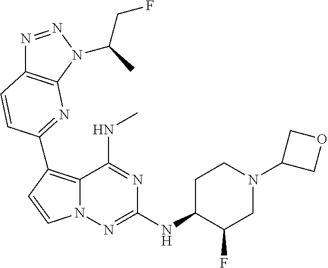 [SMILES: CNc1nc(N[C@H]2CCN(C3COC3)C[C@H]2F)nn2ccc(-c3ccc4nnn([C@H](C)CF)c4n3)c12] |
| 362 | 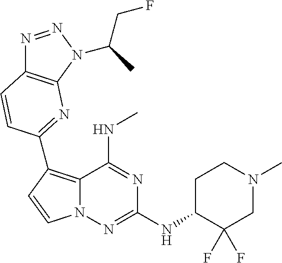 [SMILES: CNc1nc(N[C@@H]2CCN(C)CC2(F)F)nn2ccc(-c3ccc4nnn([C@H](C)CF)c4n3)c12] |
| 363 | 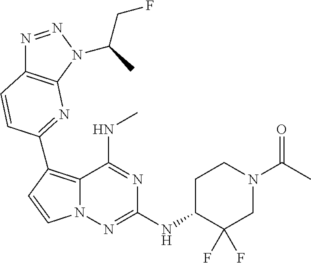 [SMILES: CNc1nc(N[C@@H]2CCN(C(C)=O)CC2(F)F)nn2ccc(-c3ccc4nnn([C@H](C)CF)c4n3)c12] |
| 364 | 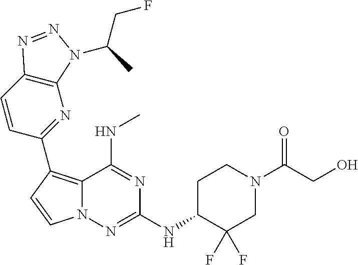 [SMILES: CNc1nc(N[C@@H]2CCN(C(=O)CO)CC2(F)F)nn2ccc(-c3ccc4nnn([C@H](C)CF)c4n3)c12] |
| 365 | 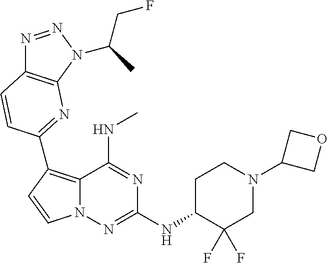 [SMILES: CNc1nc(N[C@@H]2CCN(C3COC3)CC2(F)F)nn2ccc(-c3ccc4nnn([C@H](C)CF)c4n3)c12] |
| 366 | 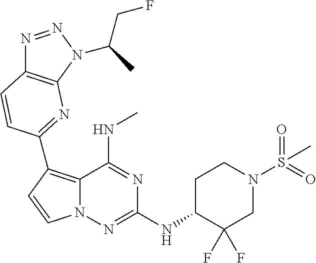 [SMILES: CNc1nc(N[C@@H]2CCN(S(C)(=O)=O)CC2(F)F)nn2ccc(-c3ccc4nnn([C@H](C)CF)c4n3)c12] |
| 367 | 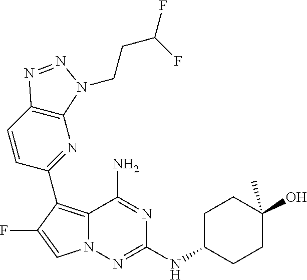 [SMILES: C[C@]1(O)CC[C@H](Nc2nc(N)c3c(-c4ccc5nnn(CCC(F)F)c5n4)c(F)cn3n2)CC1] |
| 368 | 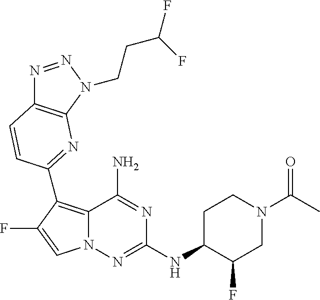 [SMILES: CC(=O)N1CC[C@H](Nc2nc(N)c3c(-c4ccc5nnn(CCC(F)F)c5n4)c(F)cn3n2)[C@H](F)C1] |
| 369 | 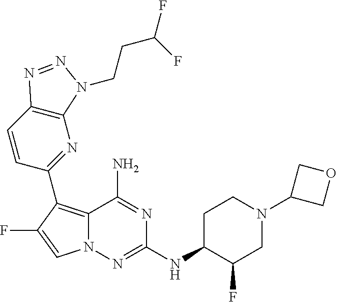 [SMILES: Nc1nc(N[C@H]2CCN(C3COC3)C[C@H]2F)nn2cc(F)c(-c3ccc4nnn(CCC(F)F)c4n3)c12] |
| 370 | 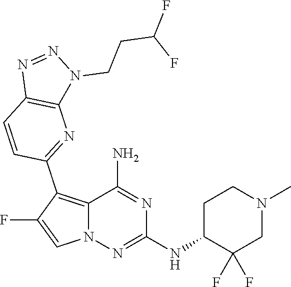 [SMILES: CN1CC[C@@H](Nc2nc(N)c3c(-c4ccc5nnn(CCC(F)F)c5n4)c(F)cn3n2)C(F)(F)C1] |
| 371 | 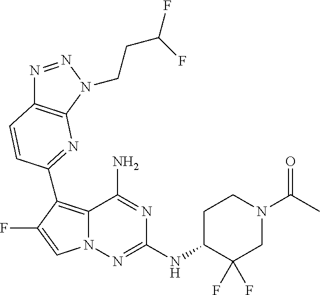 [SMILES: CC(=O)N1CC[C@@H](Nc2nc(N)c3c(-c4ccc5nnn(CCC(F)F)c5n4)c(F)cn3n2)C(F)(F)C1] |
| 372 | 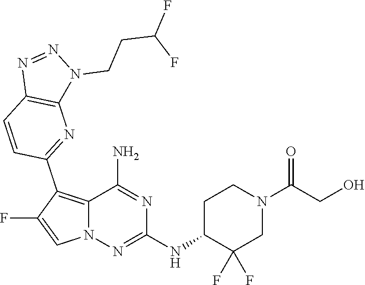 [SMILES: Nc1nc(N[C@@H]2CCN(C(=O)CO)CC2(F)F)nn2cc(F)c(-c3ccc4nnn(CCC(F)F)c4n3)c12] |
| 373 | 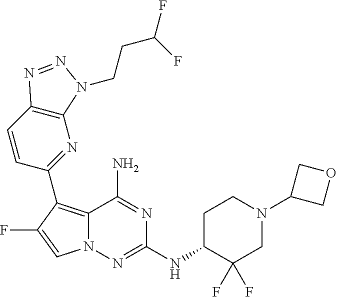 [SMILES: Nc1nc(N[C@@H]2CCN(C3COC3)CC2(F)F)nn2cc(F)c(-c3ccc4nnn(CCC(F)F)c4n3)c12] |
| 374 | 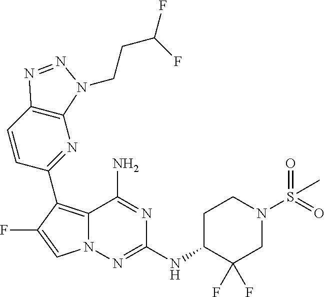 [SMILES: CS(=O)(=O)N1CC[C@@H](Nc2nc(N)c3c(-c4ccc5nnn(CCC(F)F)c5n4)c(F)cn3n2)C(F)(F)C1] |
| 375 | 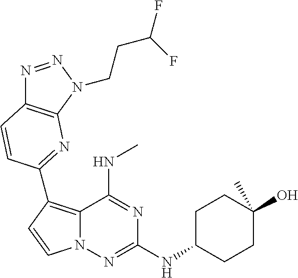 [SMILES: CNc1nc(N[C@H]2CC[C@](C)(O)CC2)nn2ccc(-c3ccc4nnn(CCC(F)F)c4n3)c12] |
| 376 | 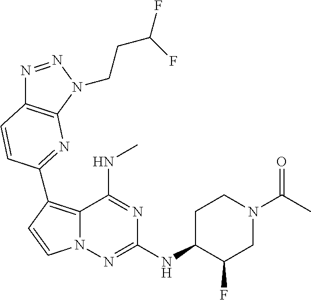 [SMILES: CNc1nc(N[C@H]2CCN(C(C)=O)C[C@H]2F)nn2ccc(-c3ccc4nnn(CCC(F)F)c4n3)c12] |
| 377 | 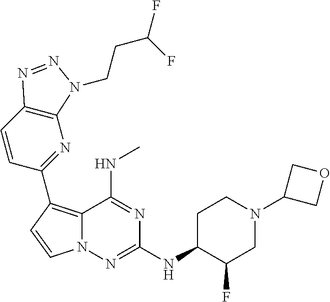 [SMILES: CNc1nc(N[C@H]2CCN(C3COC3)C[C@H]2F)nn2ccc(-c3ccc4nnn(CCC(F)F)c4n3)c12] |
| 378 | 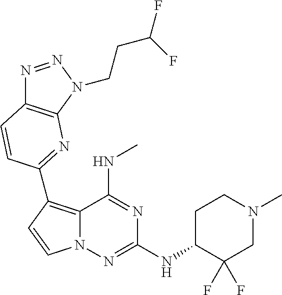 [SMILES: CNc1nc(N[C@@H]2CCN(C)CC2(F)F)nn2ccc(-c3ccc4nnn(CCC(F)F)c4n3)c12] |
| 379 | 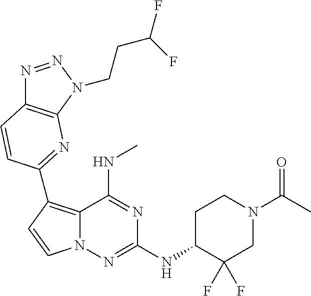 [SMILES: CNc1nc(N[C@@H]2CCN(C(C)=O)CC2(F)F)nn2ccc(-c3ccc4nnn(CCC(F)F)c4n3)c12] |
| 380 | 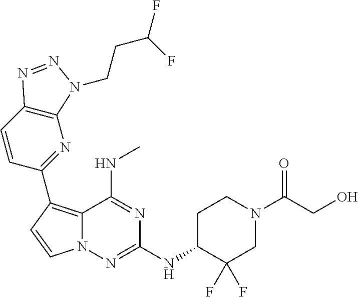 [SMILES: CNc1nc(N[C@@H]2CCN(C(=O)CO)CC2(F)F)nn2ccc(-c3ccc4nnn(CCC(F)F)c4n3)c12] |
| 381 | 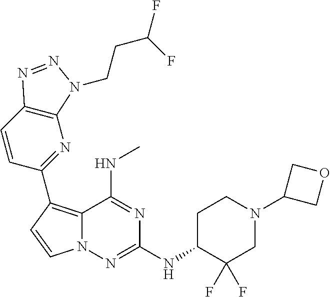 [SMILES: CNc1nc(N[C@@H]2CCN(C3COC3)CC2(F)F)nn2ccc(-c3ccc4nnn(CCC(F)F)c4n3)c12] |
| 382 | 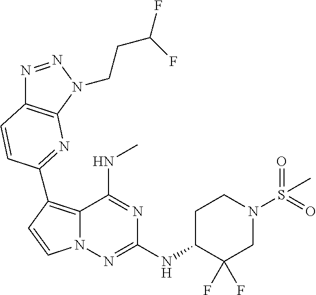 [SMILES: CNc1nc(N[C@@H]2CCN(S(C)(=O)=O)CC2(F)F)nn2ccc(-c3ccc4nnn(CCC(F)F)c4n3)c12] |
| 383 | 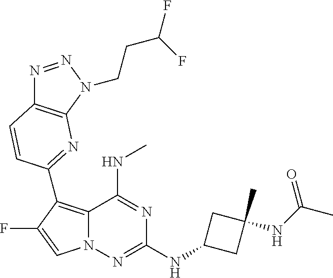 [SMILES: CNc1nc(N[C@H]2C[C@@](C)(NC(C)=O)C2)nn2cc(F)c(-c3ccc4nnn(CCC(F)F)c4n3)c12] |
| 384 | 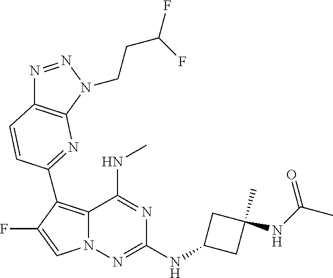 [SMILES: CNc1nc(N[C@H]2C[C@](C)(NC(C)=O)C2)nn2cc(F)c(-c3ccc4nnn(CCC(F)F)c4n3)c12] |
| 385 | 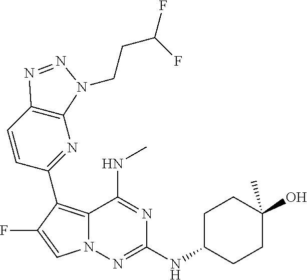 [SMILES: CNc1nc(N[C@H]2CC[C@](C)(O)CC2)nn2cc(F)c(-c3ccc4nnn(CCC(F)F)c4n3)c12] |
| 386 | 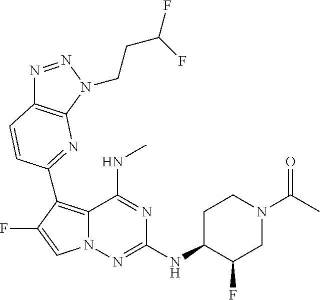 [SMILES: CNc1nc(N[C@H]2CCN(C(C)=O)C[C@H]2F)nn2cc(F)c(-c3ccc4nnn(CCC(F)F)c4n3)c12] |
| 387 | 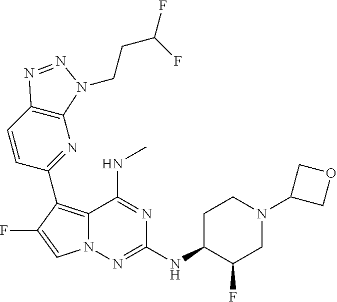 [SMILES: CNc1nc(N[C@H]2CCN(C3COC3)C[C@H]2F)nn2cc(F)c(-c3ccc4nnn(CCC(F)F)c4n3)c12] |
| 388 | 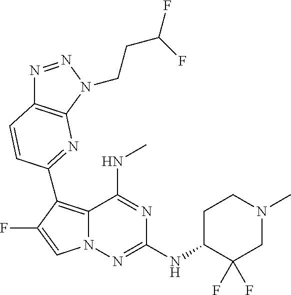 [SMILES: CNc1nc(N[C@@H]2CCN(C)CC2(F)F)nn2cc(F)c(-c3ccc4nnn(CCC(F)F)c4n3)c12] |
| 389 | 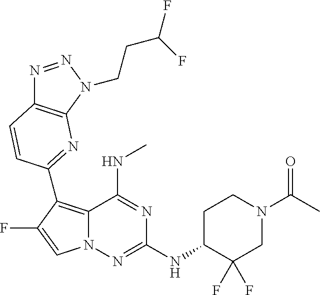 [SMILES: CNc1nc(N[C@@H]2CCN(C(C)=O)CC2(F)F)nn2cc(F)c(-c3ccc4nnn(CCC(F)F)c4n3)c12] |
| 390 | 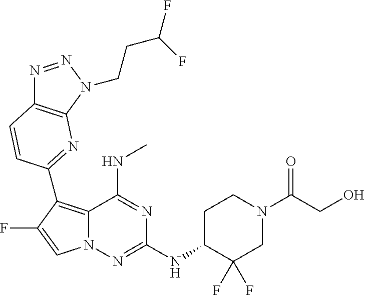 [SMILES: CNc1nc(N[C@@H]2CCN(C(=O)CO)CC2(F)F)nn2cc(F)c(-c3ccc4nnn(CCC(F)F)c4n3)c12] |
| 391 | 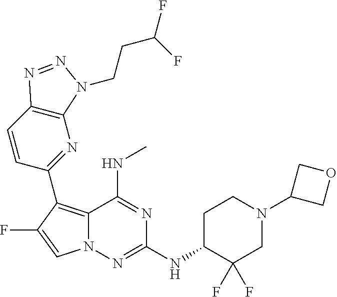 [SMILES: CNc1nc(N[C@@H]2CCN(C3COC3)CC2(F)F)nn2cc(F)c(-c3ccc4nnn(CCC(F)F)c4n3)c12] |
| 392 | 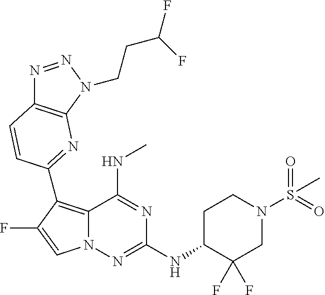 [SMILES: CNc1nc(N[C@@H]2CCN(S(C)(=O)=O)CC2(F)F)nn2cc(F)c(-c3ccc4nnn(CCC(F)F)c4n3)c12] |
| 393 | 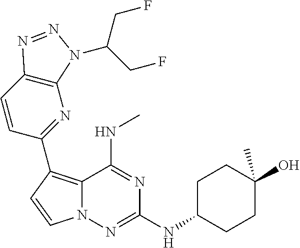 [SMILES: CNc1nc(N[C@H]2CC[C@](C)(O)CC2)nn2ccc(-c3ccc4nnn(C(CF)CF)c4n3)c12] |
| 394 | 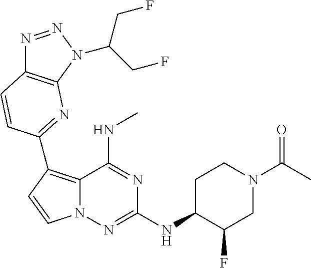 [SMILES: CNc1nc(N[C@H]2CCN(C(C)=O)C[C@H]2F)nn2ccc(-c3ccc4nnn(C(CF)CF)c4n3)c12] |
| 395 | 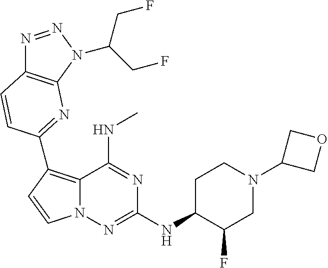 [SMILES: CNc1nc(N[C@H]2CCN(C3COC3)C[C@H]2F)nn2ccc(-c3ccc4nnn(C(CF)CF)c4n3)c12] |
| 396 | 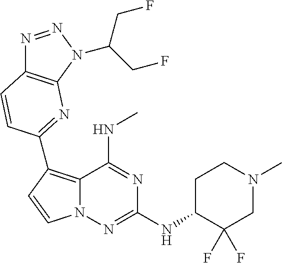 [SMILES: CNc1nc(N[C@@H]2CCN(C)CC2(F)F)nn2ccc(-c3ccc4nnn(C(CF)CF)c4n3)c12] |
| 397 | 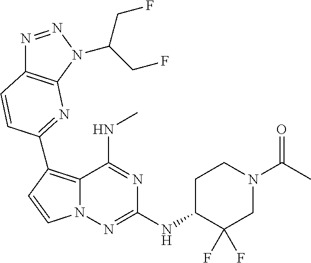 [SMILES: CNc1nc(N[C@@H]2CCN(C(C)=O)CC2(F)F)nn2ccc(-c3ccc4nnn(C(CF)CF)c4n3)c12] |
| 398 | 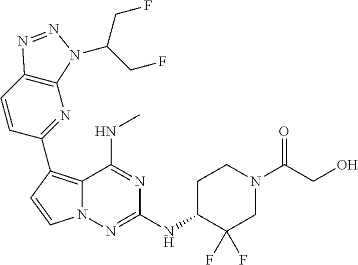 [SMILES: CNc1nc(N[C@@H]2CCN(C(=O)CO)CC2(F)F)nn2ccc(-c3ccc4nnn(C(CF)CF)c4n3)c12] |
| 399 | 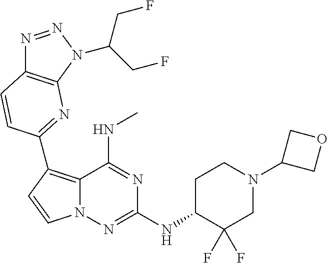 [SMILES: CNc1nc(N[C@@H]2CCN(C3COC3)CC2(F)F)nn2ccc(-c3ccc4nnn(C(CF)CF)c4n3)c12] |
| 400 | 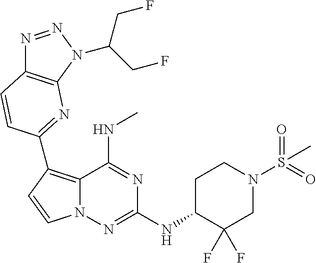 [SMILES: CNc1nc(N[C@@H]2CCN(S(C)(=O)=O)CC2(F)F)nn2ccc(-c3ccc4nnn(C(CF)CF)c4n3)c12] |
| 401 | 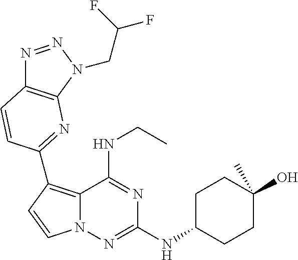 [SMILES: CCNc1nc(N[C@H]2CC[C@](C)(O)CC2)nn2ccc(-c3ccc4nnn(CC(F)F)c4n3)c12] |
| 402 | 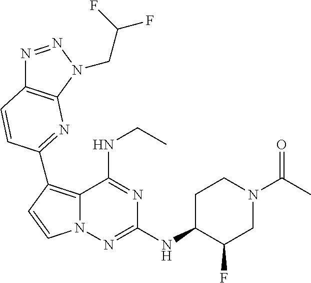 [SMILES: CCNc1nc(N[C@H]2CCN(C(C)=O)C[C@H]2F)nn2ccc(-c3ccc4nnn(CC(F)F)c4n3)c12] |
| 403 | 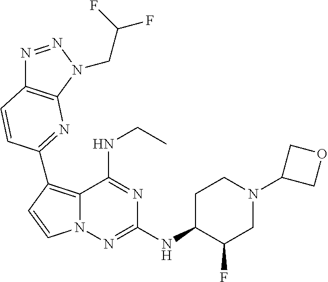 [SMILES: CCNc1nc(N[C@H]2CCN(C3COC3)C[C@H]2F)nn2ccc(-c3ccc4nnn(CC(F)F)c4n3)c12] |
| 404 | 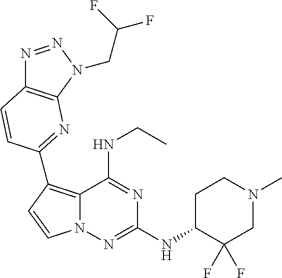 [SMILES: CCNc1nc(N[C@@H]2CCN(C)CC2(F)F)nn2ccc(-c3ccc4nnn(CC(F)F)c4n3)c12] |
| 405 | 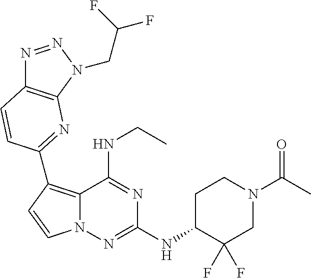 [SMILES: CCNc1nc(N[C@@H]2CCN(C(C)=O)CC2(F)F)nn2ccc(-c3ccc4nnn(CC(F)F)c4n3)c12] |
| 406 | 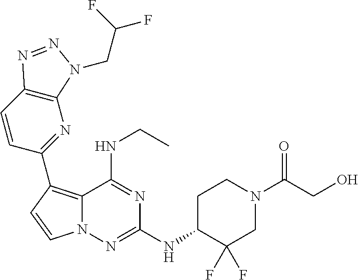 [SMILES: CCNc1nc(N[C@@H]2CCN(C(=O)CO)CC2(F)F)nn2ccc(-c3ccc4nnn(CC(F)F)c4n3)c12] |
| 407 | 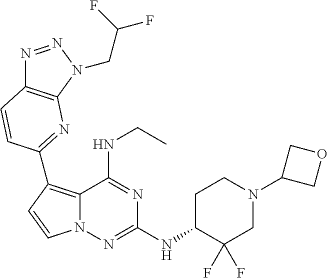 [SMILES: CCNc1nc(N[C@@H]2CCN(C3COC3)CC2(F)F)nn2ccc(-c3ccc4nnn(CC(F)F)c4n3)c12] |
| 408 | 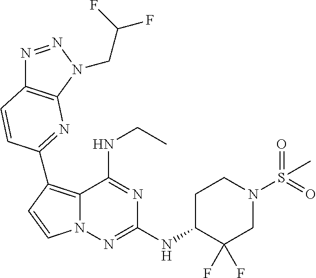 [SMILES: CCNc1nc(N[C@@H]2CCN(S(C)(=O)=O)CC2(F)F)nn2ccc(-c3ccc4nnn(CC(F)F)c4n3)c12] |
| 409 | 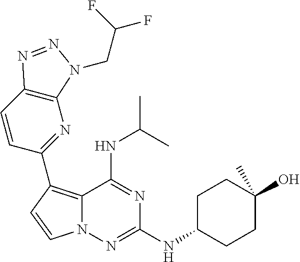 [SMILES: CC(C)Nc1nc(N[C@H]2CC[C@](C)(O)CC2)nn2ccc(-c3ccc4nnn(CC(F)F)c4n3)c12] |
| 410 | 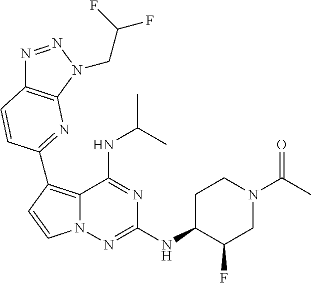 [SMILES: CC(=O)N1CC[C@H](Nc2nc(NC(C)C)c3c(-c4ccc5nnn(CC(F)F)c5n4)ccn3n2)[C@H](F)C1] |
| 411 | 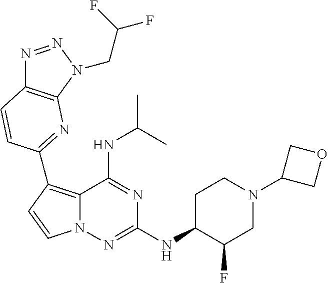 [SMILES: CC(C)Nc1nc(N[C@H]2CCN(C3COC3)C[C@H]2F)nn2ccc(-c3ccc4nnn(CC(F)F)c4n3)c12] |
| 412 | 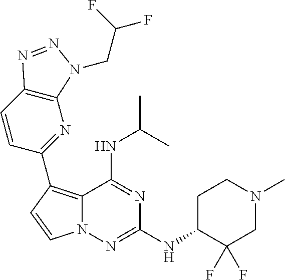 [SMILES: CC(C)Nc1nc(N[C@@H]2CCN(C)CC2(F)F)nn2ccc(-c3ccc4nnn(CC(F)F)c4n3)c12] |
| 413 | 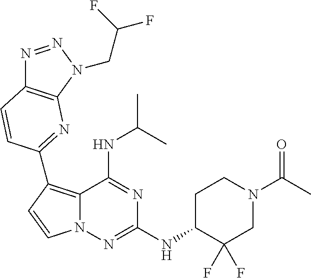 [SMILES: CC(=O)N1CC[C@@H](Nc2nc(NC(C)C)c3c(-c4ccc5nnn(CC(F)F)c5n4)ccn3n2)C(F)(F)C1] |
| 414 | 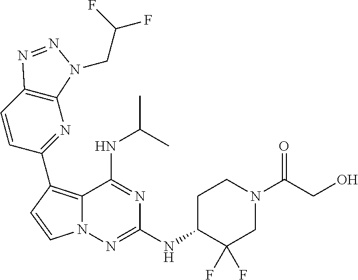 [SMILES: CC(C)Nc1nc(N[C@@H]2CCN(C(=O)CO)CC2(F)F)nn2ccc(-c3ccc4nnn(CC(F)F)c4n3)c12] |
| 415 | 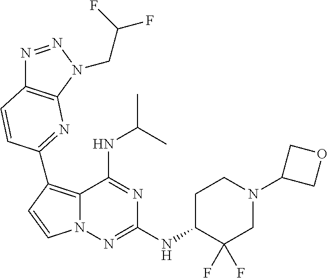 [SMILES: CC(C)Nc1nc(N[C@@H]2CCN(C3COC3)CC2(F)F)nn2ccc(-c3ccc4nnn(CC(F)F)c4n3)c12] |
| 416 | 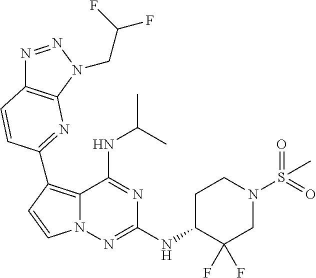 [SMILES: CC(C)Nc1nc(N[C@@H]2CCN(S(C)(=O)=O)CC2(F)F)nn2ccc(-c3ccc4nnn(CC(F)F)c4n3)c12] |
| 417 | 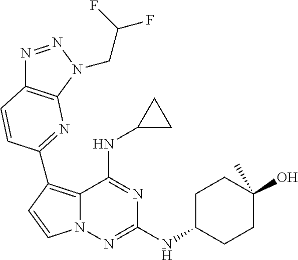 [SMILES: C[C@]1(O)CC[C@H](Nc2nc(NC3CC3)c3c(-c4ccc5nnn(CC(F)F)c5n4)ccn3n2)CC1] |
| 418 | 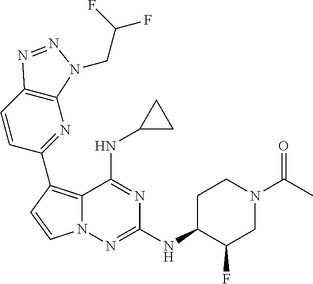 [SMILES: CC(=O)N1CC[C@H](Nc2nc(NC3CC3)c3c(-c4ccc5nnn(CC(F)F)c5n4)ccn3n2)[C@H](F)C1] |
| 419 | 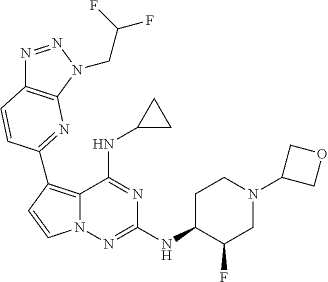 [SMILES: FC(F)Cn1nnc2ccc(-c3ccn4nc(N[C@H]5CCN(C6COC6)C[C@H]5F)nc(NC5CC5)c34)nc21] |
| 420 | 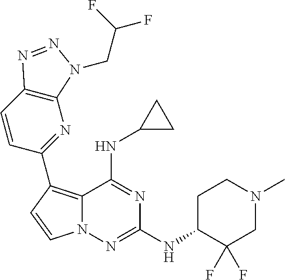 [SMILES: CN1CC[C@@H](Nc2nc(NC3CC3)c3c(-c4ccc5nnn(CC(F)F)c5n4)ccn3n2)C(F)(F)C1] |
| 421 | 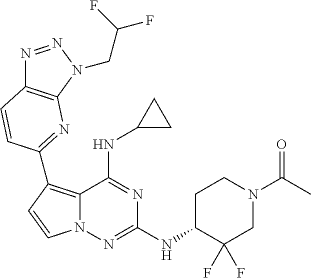 [SMILES: CC(=O)N1CC[C@@H](Nc2nc(NC3CC3)c3c(-c4ccc5nnn(CC(F)F)c5n4)ccn3n2)C(F)(F)C1] |
| 422 | 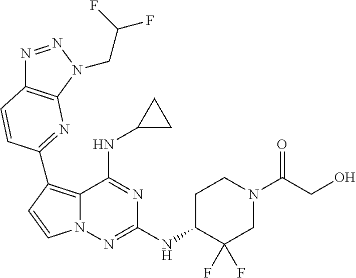 [SMILES: O=C(CO)N1CC[C@@H](Nc2nc(NC3CC3)c3c(-c4ccc5nnn(CC(F)F)c5n4)ccn3n2)C(F)(F)C1] |
| 423 | 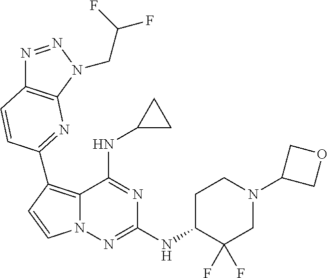 [SMILES: FC(F)Cn1nnc2ccc(-c3ccn4nc(N[C@@H]5CCN(C6COC6)CC5(F)F)nc(NC5CC5)c34)nc21] |
| 424 | 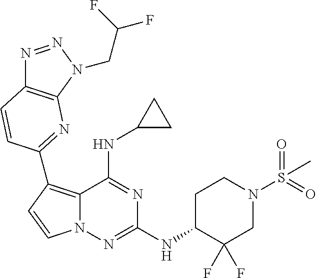 [SMILES: CS(=O)(=O)N1CC[C@@H](Nc2nc(NC3CC3)c3c(-c4ccc5nnn(CC(F)F)c5n4)ccn3n2)C(F)(F)C1] |

### Administration and Pharmaceutical Compositions

Some embodiments include pharmaceutical compositions comprising: (a) a therapeutically effective amount of a compound provided herein, or its corresponding enantiomer, diastereoisomer or tautomer, or pharmaceutically acceptable salt; and (b) a pharmaceutically acceptable carrier.

The compounds provided herein may also be useful in combination (administered together or sequentially) with other known agents.

Non-limiting examples of diseases which can be treated with a combination of a compound of Formula (I) and another active agent are colorectal cancer, ovarian cancer, hepatocellular carcinoma, head and neck squamous cell carcinoma, acute lymphoblastic leukemia (ALL), pancreatic cancer, brain tumors, acute megakaryoblastic leukemia (AMKL), and osteoarthritis. For example, a compound of Formula (I) can be combined with one or more chemotherapeutic compounds.

In some embodiments, hepatocellular carcinoma can be treated with a combination of a compound of Formula (I) and one or more of the following drugs/therapies: sorafenib (Nexavar®); regorafenib (Stivarga®, Regonix®), nivolumab (Opdivo®); lenvatinib (Lenvima®); Pembrolizumab (Keytruda®); cabozantinib (Cometriq©, Cabometyx®); 5-fluorouracil (5-FU®); ramucirumab (Cyramza®); combination of gemcitabine and oxaliplatin (GEMOX). Other therapies that can be performed in combination with a compound of Formula (I) are i) transcatheter arterial chemoembolization (TACE) in combination with doxorubicin (DOXIL®), cisplatin, or mitomycin C (Mitosol®, Mutamycin®, Jelmyto®); ii) low-dose brachytherapy.

In some embodiments, head and neck squamous cell carcinoma can be treated with a combination of a compound of Formula (I) and one or more of the following drugs/therapies: TransOral Robotic Surgery (TORS); TORS with radiation therapy; larotrectinib (Vitrakvi®); EGFR inhibitors, e.g., erlotinib (Tarceva®), osimertinib (Tagrisso®), neratinib (Nerlynx®), gefitinib (Iressa®), cetuximab (Erbitux®), panitumumab (Vectibix®), dacomitinib (Vizimpro®), lapatinib (Tykerb®), necitumumab (Portrazza), and vandetanib (Caprelsa®).

In some embodiments, acute lymphoblastic leukemia (ALL) can be treated with a combination of a compound of Formula (I) and one or more of the following drugs/therapies: remission induction therapy; consolidation therapy; nelarabine (Arranon®); Asparaginase Erwinia Chrysanthemi (Erwinaze®); Asparaginase Erwinia Chrysanthemi (Recombinant)-rywn (Rylaze®); calaspargase Pegol-mknl (Asparlas®); inotuzumab ozogamicin (Besponsa®); blinatumomab (Blincyto®); daunorubicin hydrochloride (Cerubidine®); clofarabine (Clolar®); cyclophosphamide; methotrexate sodium (Trexall®); cytarabine (Cytosar-U®); dasatinib (Sprycel®); dexamethasone; imatinib mesylate (Gleevec®); ponatinib hydrochloride (Iclusig®); mercaptopurine (Purinethol®, Purixan®); tisagenlecleucel (Kymriah®); vincristine sulfate liposome (Marqibo®); pegaspargase (Oncaspar®); prednisone; daunorubicin hydrochloride (Rubidomycin®); and vincristine sulfate.

In some embodiments, pancreatic cancer can be treated with a combination of a compound of Formula (I) and one or more of the following drugs/therapies: ablation and embolization treatment; gemcitabine (Gemzar®); 5-fluorouracil (5-FU®); oxaliplatin (Eloxatin®); albumin-bound paclitaxel (Abraxane®); capecitabine (Xeloda®); cisplatin; irinotecan (Camptosar®); liposomal Irinotecan (Onivyde®); paclitaxel (Taxol®), and docetaxel (Taxotere®).

In some embodiments, brain tumors can be treated with a combination of a compound of Formula (I) and one or more of the following drugs/therapies: carmustine can be administered by way of a gliadel wafer; for glioblastoma and high-grade glioma, radiation therapy with daily low-dose temozolomide (Temodar®) followed by monthly doses of temozolomide after radiation therapy for 6 months to 1 year; lomustine (Gleostine®), procarbazine (Matulane®), and vincristine (Vincasar®), have been used along with radiation therapy; anti-angiogenesis therapy with bevacizumab (Avastin®, Mvasi®); and targeted therapy using larotrectinib (Vitrakvi®).

In some embodiments, acute megakaryoblastic leukemia (AMKL) can be treated with a combination of a compound of Formula (I) and one or more of the following drugs/therapies: cytarabine (Cytosar-U®), etoposide (Vepesid®), and anthracycline drugs. Anthracyclines include daunorubicin (Cerubidine®), idarubicin (Idamycin®), and mitoxantrone (Novantrone®).

In some embodiments, acute myeloid leukemia (AML) can be treated with a combination of a compound of Formula (I) and one or more of the following drugs/therapies: venetoclax and hypomethylating agents (e.g., decitabine, azacitidine), induction chemotherapy (cytarabine and an anthracycline (e.g., daunorubicin or idarubicin), all-trans-retinoic acid (ATRA) and either arsenic trioxide (ATO) monotherapy or an anthracycline), consolidation therapy (cytarabine).

In some embodiments, myelodysplastic syndrome (MDS) can be treated with a combination of a compound of Formula (I) and one or more of the following drugs/therapies: 5-azacytidine, decitabine, lenalidomide, and decitabine/cedazuridine (Ingovi®).

In some embodiments, colorectal cancer can be treated with a combination of a compound of Formula (I) and one or more of the following drugs: 5-Fluorouracil (5-FU), which can be administered with the vitamin-like drug leucovorin (also called folinic acid); capecitabine (XELODA®), irinotecan (CAMPOSTAR®), oxaliplatin (ELOXATIN®). Examples of combinations of these drugs which could be further combined with a compound of Formula (I) are FOLFOX (5-FU, leucovorin, and oxaliplatin), FOLFIRI (5-FU, leucovorin, and irinotecan), FOLFOXIRI (leucovorin, 5-FU, oxaliplatin, and irinotecan) and CapeOx (Capecitabine and oxaliplatin). For rectal cancer, chemo with 5-FU or capecitabine combined with radiation may be given before surgery (neoadjuvant treatment).

In some embodiments, ovarian cancer can be treated with a combination of a compound of Formula (I) and one or more of the following drugs: Topotecan, Liposomal doxorubicin (DOXIL®), Gemcitabine (GEMZAR®), Cyclophosphamide (CYTOXAN®), Vinorelbine (NAVELBINE®), Ifosfamide (IFEX®), Etoposide (VP-16), Altretamine (HEXALEN®), Capecitabine (XELODA®), Irinotecan (CPT-11, CAMPTOSAR®), Melphalan, Pemetrexed (ALIMTA®) and Albumin bound paclitaxel (nab-paclitaxel, ABRAXANE®). Examples of combinations of these drugs which could be further combined with a compound of Formula (I) are TIP (paclitaxel [Taxol], ifosfamide, and cisplatin), VeIP (vinblastine, ifosfamide, and cisplatin) and VIP (etoposide [VP-16], ifosfamide, and cisplatin). Ovarian cancer can also be treated with a combination of a compound of Formula (I) and immune checkpoint blockade (ICB) therapy.

In some embodiments, a compound of Formula (I) can be used to treat cancer in combination with any of the following methods: (a) Hormone therapy such as aromatase inhibitors, LHRH [luteinizing hormone-releasing hormone] analogs and inhibitors, and others; (b) Ablation or embolization procedures such as radiofrequency ablation (RFA), ethanol (alcohol) ablation, microwave thermotherapy and cryosurgery (cryotherapy); (c) Chemotherapy using alkylating agents such as cisplatin and carboplatin, oxaliplatin, mechlorethamine, cyclophosphamide, chlorambucil and ifosfamide; (d) Chemotherapy using anti-metabolites such as azathioprine and mercaptopurine; (e) Chemotherapy using plant alkaloids and terpenoids such as vinca alkaloids (i.e. Vincristine, Vinblastine, Vinorelbine and Vindesine) and taxanes; (f) Chemotherapy using podophyllotoxin, etoposide, teniposide and docetaxel; (g) Chemotherapy using topoisomerase inhibitors such as irinotecan, topotecan, amsacrine, etoposide, etoposide phosphate, and teniposide; (h) Chemotherapy using cytotoxic antibiotics such as actinomycin, anthracyclines, doxorubicin, daunorubicin, valrubicin, idarubicin, epirubicin, bleomycin, plicamycin and mitomycin; (i) Chemotherapy using tyrosine-kinase inhibitors such as Imatinib mesylate (GLEEVEC®, also known as STI-571), Gefitinib (Iressa, also known as ZD1839), Erlotinib (marketed as TARCEVA®), Bortezomib (VELCADE®), tamoxifen, tofacitinib, crizotinib, Bcl-2 inhibitors (e.g. obatoclax, navitoclax (ABT-263), oblimersen (G3139), venetoclax (ABT-199), Gossypol), PARP inhibitors (e.g. Iniparib, Olaparib, Rucaparib, Niraparib, Talazoparib), PI3K inhibitors (e.g. perifosine in a phase III trial), VEGF Receptor 2 inhibitors (e.g. Apatinib), AN-152, (AEZS-108), Braf inhibitors (e.g. vemurafenib, dabrafenib and LGX818), MEK inhibitors (e.g. trametinib and MEK162), CDK inhibitors, (e.g. PD-0332991), salinomycin and Sorafenib; (j) Chemotherapy using monoclonal antibodies such as Rituximab (marketed as MABTHERA® or RITUXAN®), Trastuzumab (Herceptin also known as ErbB2), Cetuximab (marketed as ERBITUX®), and Bevacizumab (marketed as AVASTIN®); (k) Chemotherapy using KRAS G12C inhibitors such as sotorasib (Lumakras® and Lumykras®), adagrasib (MRTX849), and ARS-3248 (Wellspring Biosciences); (1) Chemotherapy using checkpoint inhibitor therapy such as Ipilimumab (Yervoy®), Nivolumab (Opdivo®), Pembrolizumab (Keytruda®), Atezolizumab (Tecentriq®), Avelumab (Bavencio), Durvalumab (Imfinzi), Cemiplimab (Libtayo®), and Spartalizumab (PDR001); (m) Chemotherapy using antibody-drug conjugates (ADC) such as Gemtuzumab ozogamicin, Brentuximab vedotin, Trastuzumab emtansine, Inotuzumab ozogamicin, Polatuzumab vedotin, Enfortumab vedotin, Trastuzumab deruxtecan, Sacituzumab govitecan, Belantamab mafodotin, Moxetumomab pasudotox, and Loncastuximab tesirine; (n) Chemotherapy using proteasome inhibitors such as carfilzomib, lactacystin, disulfiram, salinosporamide A (marizomib), oprozomib, delanzomib, epoxomicin, MG132, β-hydroxy γ-methylbutyric acid (HMB), bortezomib, ixazomib (alone or in in combination with lenalidomide and dexamethasone); and (o) radiation therapy.

In some embodiments, a compound of Formula I, can be used to treat diabetes mellitus in combination with any of the following methods: (a) injections of insulin; (b) biguanides such as metformin (Glucophage), phenformin (DBI), and buformin; (c) thiazolidinediones (TZDs) such as rosiglitazone (Avandia), pioglitazone (Actos), and yroglitazone (Rezulin); (d) lyn kinase activators such as glimepiride (Amaryl®) and tolimidone (MLR-1023); (e) secretagogues such as sulfonylureas (non-limiting examples are acetohexamide, carbutamide, chlorpropamide, glycyclamide (tolcyclamide), metahexamide, tolazamide, tolbutamide, glibenclamide (glyburide), glibornuride, gliclazide, glipizide, gliquidone, glisoxepide, glyclopyramide, and glimepiride) and meglitinides (nonlimiting examples are repaglinide (Prandin), nateglinide (Starlix), and mitiglinide (Glufast)); (f) alpha-glucosidase inhibitors such as acarbose (Glucobay, Precose, Prandase), miglitol (Glyset), and voglibose; (g) injectable incretin mimetics such as glucagon-like peptide-1 (GLP-1) and gastric inhibitory peptide (glucose-dependent insulinotropic peptide, GIP), nonlimiting examples of injectable glucagon-like peptide (GLP) analogs and agonists are exenatide (Exendin-4, marketed as Byetta), liraglutide (Victoza, Saxenda), taspoglutide, lixisenatide (Lyxumia), Semaglutide (Ozempic, Rybelsus), dulaglutide (Trulicity), albiglutide (Tanzeum), nonlimiting examples of dipeptidyl peptidase-4 (DPP-4) inhibitors are sitagliptin (Januvia), vildagliptin (Galvus), saxagliptin (Onglyza), linagliptin (Tradjenta), gemigliptin (Zemiglo), anagliptin (Suiny), teneligliptin (Tenelia), alogliptin (Nesina, Vipidia, Kazano, Vipidomet (with metformin), Oseni, Incresync (with pioglitazone)), trelagliptin (Zafatek, Wedica), omarigliptin (MK-3102), evogliptin (Suganon, Evodine), gosogliptin (Saterex), and dutogliptin; (h) injectable amylin analogues such as pramlintide (Symlin); (i) glycosurics (SGLT2 inhibitors) such as canagliflozin (Invokana, Sulisent, Prominad), dapagliflozin (Forxiga, Farxiga, Edistride), empagliflozin (Jardiance, Sciampa-M), ertugliflozin (Steglatro), ipragliflozin (Suglat), luseogliflozin (Lusefi), remogliflozin etabonate (pro-drug of remogliflozin), sergliflozin etabonate (GW869682X), sotagliflozin (Zynquista), and tofogliflozin (CSG452).

In some embodiments, a compound of Formula (I) can be used to treat osteoarthritis in combination with any of the following methods: (d) injections of a Wnt signaling pathway inhibitor (e.g. lorecivivint); (a) Nonsteroidal anti-inflammatory drugs (NSAIDs) such as ibuprofen, naproxen, aspirin and acetaminophen; (b) physical therapy; (c) injections of corticosteroid medications; (d) injections of hyaluronic acid derivatives (e.g. Hyalgan, Synvisc); (e) narcotics, like codeine; (f) in combination with braces and/or shoe inserts or any device that can immobilize or support your joint to help you keep pressure off it (e.g., splints, braces, shoe inserts or other medical devices); (g) realigning bones (osteotomy); (h) joint replacement (arthroplasty); and (i) in combination with a chronic pain class.

In some embodiments, a compound of Formula (I) can be used to treat Alzheimer's disease in combination with aducanumab (Aduhelm™); acetylcholinesterase inhibitors, e.g., tacrine, rivastigmine (Exelon®), galantamine (Razadyne® and GalantaMind™), and donepezil (Aricept®); and memantine (Axura®, Ebixa®, Namenda®).

Administration of the compounds disclosed herein or the pharmaceutically acceptable salts thereof can be via any of the accepted modes of administration, including, but not limited to, orally, subcutaneously, intravenously, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic devices. In some embodiments, the administration method includes oral or parenteral administration.

Compounds provided herein intended for pharmaceutical use may be administered as crystalline or amorphous products. Pharmaceutically acceptable compositions may include solid, semi-solid, liquid, solutions, colloidal, liposomes, emulsions, suspensions, complexes, coacervates and aerosols. Dosage forms, such as, e.g., tablets, capsules, powders, liquids, suspensions, suppositories, aerosols, implants, controlled release, or the like. They may be obtained, for example, as solid plugs, powders, or films by methods such as precipitation, crystallization, milling, grinding, supercritical fluid processing, coacervation, complex coacervation, encapsulation, emulsification, complexation, freeze drying, spray drying, or evaporative drying. Microwave or radio frequency drying may be used for this purpose. The compounds can also be administered in sustained or controlled release dosage forms, including depot injections, osmotic pumps, pills (tablets and or capsules), transdermal (including electrotransport) patches, implants, and the like, for prolonged and/or timed, pulsed administration at a predetermined rate.

The compounds can be administered either alone or in combination with a conventional pharmaceutical carrier, excipient, or the like. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic carrier may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In one embodiment, the compositions will take the form of a unit dosage form such as a pill or tablet and thus the composition may contain, along with a compound provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol, or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

Injectables can be prepared in conventional forms, either as liquid solutions, colloid, liposomes, complexes, coacervate or suspensions, as emulsions, or in solid forms suitable for reconstitution in liquid prior to injection. The percentage of a compound provided herein contained in such parenteral compositions is highly dependent on the specific nature thereof, as well as the activity of the compound and the needs of the patient. However, percentages of active ingredient of 0.01% to 10% in solution are employable and could be higher if the composition is a solid or suspension, which could be subsequently diluted to the above percentages.

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In one embodiment, the compositions can be administered to the respiratory tract (including nasal and pulmonary) e.g., through a nebulizer, metered-dose inhalers, atomizer, mister, aerosol, dry powder inhaler, insufflator, liquid instillation or other suitable device or technique.

In some embodiments, aerosols intended for delivery to the nasal mucosa are provided for inhalation through the nose. For optimal delivery to the nasal cavities, inhaled particle sizes of about 5 to about 100 microns are useful, with particle sizes of about 10 to about 60 microns being preferred. For nasal delivery, a larger inhaled particle size may be desired to maximize impaction on the nasal mucosa and to minimize or prevent pulmonary deposition of the administered formulation. In some embodiments, aerosols intended for delivery to the lung are provided for inhalation through the nose or the mouth. For delivery to the lung, inhaled aerodynamic particle sizes of about less than 10 μm are useful (e.g., about 1 to about 10 microns). Inhaled particles may be defined as liquid droplets containing dissolved drug, liquid droplets containing suspended drug particles (in cases where the drug is insoluble in the suspending medium), dry particles of pure drug substance, drug substance incorporated with excipients, liposomes, emulsions, colloidal systems, coacervates, aggregates of drug nanoparticles, or dry particles of a diluent which contain embedded drug nanoparticles.

In some embodiments, compounds of Formula (I) disclosed herein intended for respiratory delivery (either systemic or local) can be administered as aqueous formulations, as non-aqueous solutions, or suspensions, as suspensions or solutions in halogenated hydrocarbon propellants with or without alcohol, as a colloidal system, as emulsions, coacervates, or as dry powders. Aqueous formulations may be aerosolized by liquid nebulizers employing either hydraulic or ultrasonic atomization or by modified micropump systems (like the soft mist inhalers, the Aerodose® or the AERx® systems). Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

In some embodiments, the compositions of Formula (I) disclosed herein can be administered to the ear by various methods. For example, a round window catheter (e.g., U.S. Pat. Nos. 6,440,102 and 6,648,873) can be used.

Alternatively, formulations can be incorporated into a wick for use between the outer and middle ear (e.g., U.S. Pat. No. 6,120,484) or absorbed to collagen sponge or other solid support (e.g., U.S. Pat. No. 4,164,559).

If desired, formulations of the disclosure can be incorporated into a gel formulation (e.g., U.S. Pat. Nos. 4,474,752 and 6,911,211).

In some embodiments, compounds of Formula (I) disclosed herein intended for delivery to the ear can be administered via an implanted pump and delivery system through a needle directly into the middle or inner ear (cochlea) or through a cochlear implant stylet electrode channel or alternative prepared drug delivery channel such as but not limited to a needle through temporal bone into the cochlea.

Other options include delivery via a pump through a thin film coated onto a multichannel electrode or electrode with a specially imbedded drug delivery channel (pathways) carved into the thin film for this purpose. In other embodiments the acidic or basic solid compound of Formula (I) can be delivered from the reservoir of an external or internal implanted pumping system.

Formulations of the disclosure also can be administered to the ear by intratympanic injection into the middle ear, inner ear, or cochlea (e.g., U.S. Pat. No. 6,377,849 and Ser. No. 11/337,815).

Intratympanic injection of therapeutic agents is the technique of injecting a therapeutic agent behind the tympanic membrane into the middle and/or inner ear. In one embodiment, the formulations described herein are administered directly onto the round window membrane via transtympanic injection. In another embodiment, the ion channel modulating agent auris-acceptable formulations described herein are administered onto the round window membrane via a non-transtympanic approach to the inner ear. In additional embodiments, the formulation described herein is administered onto the round window membrane via a surgical approach to the round window membrane comprising modification of the crista fenestrae cochleae.

In some embodiments, the compounds of Formula (I) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG (like PEG ointments), and the like.

Suppositories for rectal administration of the drug (either as a solution, colloid, suspension or a complex) can be prepared by mixing a compound provided herein with a suitable non-irritating excipient that is solid at ordinary temperatures but liquid at the rectal temperature and will therefore melt or erode/dissolve in the rectum and release the compound. Such materials include cocoa butter, glycerinated gelatin, hydrogenated vegetable oils, poloxamers, mixtures of polyethylene glycols of various molecular weights and fatty acid esters of polyethylene glycol. In suppository forms of the compositions, a low-melting wax such as, but not limited to, a mixture of fatty acid glycerides, optionally in combination with cocoa butter, is first melted.

Solid compositions can be provided in various different types of dosage forms, depending on the physicochemical properties of the compound provided herein, the desired dissolution rate, cost considerations, and other criteria. In one of the embodiments, the solid composition is a single unit. This implies that one unit dose of the compound is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In one embodiment, the solid composition is a highly porous lyophilized form. Such lyophilizates, sometimes also called wafers or lyophilized tablets, are particularly useful for their rapid disintegration, which also enables the rapid dissolution of the compound.

On the other hand, for some applications the solid composition may also be formed as a multiple unit dosage form as defined above. Examples of multiple units are powders, granules, microparticles, pellets, mini-tablets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution. Effervescent compositions are also contemplated to aid the quick dispersion and absorption of the compound.

Another type of multiparticulate system which is also capable of achieving rapid drug dissolution is that of powders, granules, or pellets from water-soluble excipients which are coated with a compound provided herein so that the compound is located at the outer surface of the individual particles. In this type of system, the water-soluble low molecular weight excipient may be useful for preparing the cores of such coated particles, which can be subsequently coated with a coating composition comprising the compound and, for example, one or more additional excipients, such as a binder, a pore former, a saccharide, a sugar alcohol, a film-forming polymer, a plasticizer, or other excipients used in pharmaceutical coating compositions.

Also provided herein are kits. Typically, a kit includes one or more compounds or compositions as described herein. In certain embodiments, a kit can include one or more delivery systems, e.g., for delivering or administering a compound as provided herein, and directions for use of the kit (e.g., instructions for treating a patient). In another embodiment, the kit can include a compound or composition as described herein and a label that indicates that the contents are to be administered to a patient with cancer. In another embodiment, the kit can include a compound or composition as described herein and a label that indicates that the contents are to be administered to a patient with one or more of glioblastoma, ovarian, breast, pancreatic cancers, acute lymphoblastic leukemia, acute megakaryoblastic leukemia, chronic myeloid leukemia, Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, Autism, Dementia, Epilepsy, Huntington's Disease, and Multiple Sclerosis.

### Methods of Treatment

The compounds and compositions provided herein can be used as inhibitors of DYRK1A, and thus can be used to treat a variety of disorders and diseases in which over expression of DYRK1A is implicated, such as cancer and neurological conditions/disorders/diseases. Non-limiting examples of diseases which can be treated with the compounds and compositions provided herein include a variety of cancers, Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, Stroke, tauopathies (e.g., Pick's disease, progressive supranuclear palsy, corticobasal degeneration, argyrophilic grain disease, globular glial tauopathies, primary age-related tauopathy, which includes neurofibrillary tangle dementia, chronic traumatic encephalopathy (CTE), frontotemporal lobar degeneration with tau inclusions (FTLD-tau), and aging-related tau astrogliopathy. Clinical symptoms include frontotemporal dementia, corticobasal syndrome, Richardson syndrome, parkinsonism, pure akinesia with gait freezing and, rarely, motor neuron symptoms or cerebellar ataxia, diabetes, psoriasis, knee osteoarthritis, tendinopathy, human immunodeficiency virus type 1 (HIV-1), human cytomegalovirus (HCMV), hepatitis C virus (HCV), and herpes simplex virus 1 (HSV-1).

The gene encoding DYRK1A is located on chromosome 21, within the Down syndrome critical region (DSCR), the triploidy of which is responsible for most Down syndrome-associated deficiencies (FEBS Journal (2011), 278, 246-256). There is considerable genetical and pharmacological evidence showing that the mere 1.5-fold overexpression of DYRK1A is responsible for most cognitive deficits observed in Down syndrome patients (Pharmacology & Therapeutics (2019), 194, 199-221 and Brain Science (2018), 8(10), 187). Genetical normalization of DYRK1A levels or pharmacological inhibition of its catalytic activity restores cognitive functions. The development of pharmacological inhibitors of DYRK1A is a major avenue for the treatment of cognitive deficits associated with Down syndrome.

DYRK1A and DYRK1B are utilized during human cytomegalovirus (HCMV) placental replication. Inhibition of DYRKs prevent replication of various viruses, including hepatitis C virus (HCV), human cytomegalovirus (HCMV), human immunodeficiency virus type 1 (HIV-1), and herpes simplex virus 1 (HSV-1) (Journal of Virology (2020), 94(6) and PLoS ONE (2015), 10, e0144229).

There is a growing body of evidence showing that DYRK1A/1B inhibitors induce the proliferation of insulin-producing pancreatic β-cells, making DYRK1A/1B kinases attractive therapeutic targets for β-cell regeneration for both type 1 and type 2 diabetes mellitus and gestational diabetes (Nature Communications (2015), 6(8372); Diabetes (2016), 65(6), 1660-1671; JCI Insight (2020), 5(1), e132594; Science Translational Medicine (2020), 12(530); International Journal of Molecular Sciences (2021), 22(16), 9083; and Journal of Medicinal Chemistry (2021), 64(6), 2901-2922). Other forms of diabetes that may be treated with DYRK inhibitors are maturity onset diabetes of the young (MODY, monogenic diabetes), cases of diabetes that are caused by the body's tissue receptors not responding to insulin, double diabetes (when a type 1 diabetic becomes insulin resistant), diabetes associated with excessive secretion of insulin-antagonistic hormones, malnutrition-related diabetes mellitus (ICD-10 code E12), and diabetes caused by any genetic mutations (autosomal or mitochondrial) that leads to defects in beta cell function.

There is abundant literature linking DYRK1A with solid cancers and leukemias (Pharmacology & Therapeutics (2015), 151, 87-98; Cancers (2020), 12(8), 2106; and Cellular and Molecular Life Sciences (2021), 78, 603-619). The most prominent examples are pancreatic cancer (Gut (2019), 68(8), 1465-1476 and Gene (2020), 758, 144960), brain tumors, glioblastoma (Journal of Clinical Investigation (2013), 123(6), 2475-2487), acute megakaryoblastic leukemia (AMKL) (Journal of Clinical Investigation (2012), 122(3), 948-962), and acute lymphoblastic leukemia (ALL) (Journal of Clinical Investigation (2021), 131(1), e135937). Other cancers linked to DYRK1A are ovarian (Frontiers in Oncology (2021), 11, 637193), head and neck squamous cell carcinoma (Scientific Reports (2016), 6, 36132), hepatocellular carcinoma (Cell Death & Disease (2021), 12, 125), DYRK1A regulates DNA damage response (Scientific Reports (2019), 9, 6014 and Scientific Reports (2019), 9, 6539). In some situations, DYRK1A appears to function as a tumor-suppressor protein (Molecular & Cellular Oncology (2015), 2(1), e970048 and Nature (2016), 529, 172-177).

Other cancers can also be treated with the compounds and compositions described herein.

More particularly, cancers that may be treated by the compounds, compositions and methods described herein include, but are not limited to, the following:
1) Breast cancers, including, for example ER⁺ breast cancer, ER⁻ breast cancer, her2⁻ breast cancer, her2⁺ breast cancer, stromal tumors such as fibroadenomas, phyllodes tumors, and sarcomas, and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; chemoresistant breast cancers (TNBC), and miscellaneous malignant neoplasms. Further examples of breast cancers can include luminal A, luminal B, basal A, basal B, and triple negative breast cancer, which is estrogen receptor negative (ER⁻), progesterone receptor negative, and her2 negative (her2⁻). In some embodiments, breast cancer may have a high risk Oncotype score.
2) Cardiac cancers, including, for example sarcoma, e.g., angiosarcoma, fibrosarcoma, rhabdomyosarcoma, and liposarcoma; myxoma; rhabdomyoma; fibroma; lipoma and teratoma.
3) Lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; chemoresistant small cell lung cancer (SCLC), and mesothelioma.
4) Gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; colon cancers with APC gene mutations; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma.
5) Genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma.
6) Liver cancers, including, for example, hepatoma, e.g., hepatocellular carcinoma; cholangiocarcinoma; hepatoblastoma; angiosarcoma; hepatocellular adenoma; and hemangioma.
7) Bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors.
8) Nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, oligodendrocytoma, schwannoma, retinoblastoma, and congenital tumors; and cancers of the spinal cord, e.g., neurofibroma, meningioma, glioma, and sarcoma.
9) Gynecological cancers, including, for example, cancers of the uterus, e.g., endometrial cancers (e.g., carcinoma, endometrioid adenocarcinoma, serous carcinoma, clear cell carcinoma, mucinous carcinomas, mixed or undifferentiated carcinoma (including mixed Mhllerian tumor), endometrial stromal sarcoma, squamous cell carcinoma of the endometrium, urothelial carcinoma, endometrial cancer with CTNNB1 mutations); cancers of the cervix, e.g., cervical carcinoma, and pre tumor cervical dysplasia; cancers of the ovaries, e.g., BRCA-mutant ovarian cancer, surface epithelial-stromal tumors (epithelial ovarian cancer (Type 1 (endometroid, mucinous, clear cell, low grade serous) or Type 2 (poorly differentiated, carcinosarcoma, and high grade serous))), ovarian carcinoma, including serous cystadenocarcinoma, mucinous cystadenocarcinoma, endometrioid tumors, small cell ovarian cancer (small cell ovarian cancer of hypercalcemic type, small cell ovarian cancer of pulmonary type) unclassified carcinoma, granulosa theca cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma, primary fallopian tube cancer; Primary peritoneal cancer (also known as serous surface papillary carcinoma, primary peritoneal carcinoma, extra-ovarian serous carcinoma, primary serous papillary carcinoma, and psammomacarcinoma).
10) Hematologic cancers, including, for example, cancers of the blood, e.g., acute myeloid leukemia, chronic myeloid leukemia, myelodysplastic syndromes (refractory cytopenia with unilineage dysplasia (refractory anemia, refractory neutropenia, and refractory thrombocytopenia), refractory anemia with ring sideroblasts, refractory cytopenia with multilineage dysplasia, refractory anemias with excess blasts I and II, refractory cytopenia of childhood), and myeloproliferative neoplasms, acute lymphoblastic leukemia, chronic lymphocytic leukemia, myeloproliferative diseases, multiple myeloma, myelodysplastic syndrome, myelodysplastic-myeloproliferative diseases, Hodgkin's lymphoma, non-Hodgkin's lymphoma (malignant lymphoma) and Waldenström's macroglobulinemia.
11) Skin cancers and skin disorders, including, for example, malignant melanoma and metastatic melanoma, basal cell carcinoma, squamous cell carcinoma, Kaposi's sarcoma, moles dysplastic nevi, lipoma, angioma, dermatofibroma, keloids, and scleroderma.
12) Adrenal gland cancers, including, for example, neuroblastoma.
13) Soft-tissue sarcomas (STS) such as fibrosarcoma, malignant fibrous histiocytoma, dermatofibrosarcoma, liposarcoma, rhabdomyosarcoma, leiomyosarcoma, hemangiosarcoma, Kaposi's sarcoma, lymphangiosarcoma, synovial sarcoma, malignant peripheral nerve sheath tumors (also called neurofibrosarcomas, malignant schwannomas, and neurogenic sarcomas), neurofibrosarcoma, extraskeletal chondrosarcoma, extraskeletal osteosarcoma, extraskeletal myxoid chondrosarcoma, extraskeletal mesenchymal, embryonal, alveolar soft part sarcoma, and infantile hemangio-pericytoma.

More particularly, tumors of the central nervous system that may be treated by the compounds, compositions and methods described herein include:
1) Astrocytic tumors, e.g., diffuse astrocytoma (fibrillary, protoplasmic, gemistocytic, mixed), anaplastic (malignant) astrocytoma, glioblastoma multiforme (giant cell glioblastoma and gliosarcoma), pilocytic astrocytoma (pilomyxoid astrocytoma), pleomorphic xanthoastrocytoma, subependymal giant cell astrocytoma, and gliomatosis cerebri.
2) Oligodendroglial tumors, e.g., oligodendroglioma and anaplastic oligodendroglioma.
3) Oligoastrocytic tumors, e.g., oligoastrocytoma and anaplastic oligoastrocytoma.
4) Ependymal tumors, e.g., subependymoma, myxopapillary ependymoma, ependymoma, (cellular, papillary, clear cell, tanycytic), and anaplastic (malignant) ependymoma.
5) Choroid plexus tumors, e.g., choroid plexus papilloma, atypical choroid plexus papilloma, and choroid plexus carcinoma.
6) Neuronal and mixed neuronal-glial tumors, e.g., gangliocytoma, ganglioglioma, dysembryoplastic neuroepithelial tumor (DNET), dysplastic gangliocytoma of the cerebellum (Lhermitte-Duclos), desmoplastic infantile astrocytoma/ganglioglioma, central neurocytoma, anaplastic ganglioglioma, extraventricular neurocytoma, cerebellar liponeurocytoma, Papillary glioneuronal tumor, Rosette-forming glioneuronal tumor of the fourth ventricle, and paraganglioma of the filum terminale.
7) Pineal tumors, e.g., pineocytoma, pineoblastoma, papillary tumors of the pineal region, and pineal parenchymal tumor of intermediate differentiation.
8) Embryonal tumors, e.g., medulloblastoma (medulloblastoma with extensive nodularity, anaplastic medulloblastoma, desmoplastic, large cell, melanotic, medullomyoblastoma), medulloepithelioma, supratentorial primitive neuroectodermal tumors, and primitive neuroectodermal tumors (PNETs) such as neuroblastoma, ganglioneuroblastoma, ependymoblastoma, and atypical teratoid/rhabdoid tumor.
9) Neuroblastic tumors, e.g., olfactory (esthesioneuroblastoma), olfactory neuroepithelioma, and neuroblastomas of the adrenal gland and sympathetic nervous system.
10) Glial tumors, e.g., astroblastoma, chordoid glioma of the third ventricle, and angiocentric glioma.
11) Tumors of cranial and paraspinal nerves, e.g., schwannoma, neurofibroma Perineurioma, and malignant peripheral nerve sheath tumor.
12) Tumors of the meninges such as tumors of meningothelial cells, e.g., meningioma (atypical meningioma and anaplastic meningioma); mesenchymal tumors, e.g., lipoma, angiolipoma, hibernoma, liposarcoma, solitary fibrous tumor, fibrosarcoma, malignant fibrous histiocytoma, leiomyoma, leiomyosarcoma, rhabdomyoma, rhabdomyosarcoma, chondroma, chondrosarcoma, osteoma, osteosarcoma, osteochondroma, haemangioma, epithelioid hemangioendothelioma, haemangiopericytoma, anaplastic haemangiopericytoma, angiosarcoma, Kaposi Sarcoma, and Ewing Sarcoma; primary melanocytic lesions, e.g., diffuse melanocytosis, melanocytoma, malignant melanoma, meningeal melanomatosis; and hemangioblastomas.
13) Tumors of the hematopoietic system, e.g., malignant Lymphomas, plasmocytoma, and granulocytic sarcoma.
14) Germ cell tumors, e.g., germinoma, embryonal carcinoma, yolk sac tumor, choriocarcinoma, teratoma, and mixed germ cell tumors.
15) Tumors of the sellar region, e.g., craniopharyngioma, granular cell tumor, pituicytoma, and spindle cell oncocytoma of the adenohypophysis.

Cancers may be solid tumors that may or may not be metastatic. Cancers may also occur, as in leukemia, as a diffuse tissue. Thus, the term “tumor cell,” as provided herein, includes a cell afflicted by any one of the above identified disorders.

A method of treating cancer using a compound or composition as described herein may be combined with existing methods of treating cancers, for example by chemotherapy, irradiation, or surgery (e.g., oophorectomy). In some embodiments, a compound or composition can be administered before, during, or after another anticancer agent or treatment.

There is mounting evidence for a role of DYRK1A in the onset of Alzheimer's Disease (Future Medicinal Chemistry (2016), 8(6), 681-696 and European Journal of Medicinal Chemistry (2018), 158, 559-592). DYRK1A phosphorylates key substrates involved in Alzheimer's Disease and dementia: Tau, septin 4, amyloid precursor protein (APP), presenilin 1, neprilysin, Munc18-1, α-synuclein, RCAN1, and β-tubulin. By modulating alternative splicing of Tau exon 10, DYRK1A favors the production of the 3R-Tau splice isoform (characteristic for DS/AD/tauopathy) over the 4R-Tau isoform (Journal of Biological Chemistry (2015), 290, 15219-15237).

Genome-wide association studies (GWAS) have revealed that DYRK1A is a risk factor for Parkinson's Disease (The Lancet Neurology (2019), 18(12), 1091-1102). DYRK1A phosphorylates key factors for Parkinson's Disease such as parkin, septin 4, and α-synuclein. Upregulation of micro-RNAs specific for Parkinson's Disease targets DYRK1A expression. There is further evidence that DYRK1A expression is increased in Parkinson's Disease and in Pick's disease (Neurobiology of Disease (2005), 20(2), 392-400).

The compounds and compositions provided herein can be used as inhibitors and/or modulators of the enzyme DYRK1A, and thus can be used to treat a variety of disorders and diseases associated with tau protein, including, but not limited to, Alzheimer's disease, amyotrophic lateral sclerosis (ALS), down syndrome, frontotemporal dementia (FTD) including FTD with Parkinsonism-17 (FTDP-17), behavioural variant frontotemporal dementia (bvFTD), FTD in patients with motor neuron disease (MND) (typically amyotrophic lateral sclerosis, also called FTD-ALS), corticobasal degeneration (CBD) (also called corticobasal ganglionic degeneration), progressive supranuclear palsy, primary progressive aphasia (PPA), globular glial tauopathy (GGT), myotonic dystrophy type 1 (DM1) (also called Steinert disease), myotonic dystrophy type 2 (DM2) (also called proximal myotonic myopathy), Guam complex, argyrophilic grain disease, dementia pugilistica, post-encephalitic parkinsonism, Lewy body dementia, Parkinson's disease, Pick's disease, and additional diseases with pronounced neurodegeneration such as autism, dementia, epilepsy, Huntington's disease, multiple sclerosis; diseases and disorders associated with acquired brain injury such as chronic traumatic encephalopathy, traumatic brain injury, tumor, and stroke.

Non-limiting examples of neurological disorders (e.g., neurological conditions and neurological diseases) which can be treated with the compounds and compositions provided herein include Alzheimer's disease, aphasia, apraxia, arachnoiditis, ataxia telangiectasia, attention deficit hyperactivity disorder, auditory processing disorder, autism, alcoholism, Bell's palsy, bipolar disorder, brachial plexus injury, Canavan disease, carpal tunnel syndrome, causalgia, central pain syndrome, central pontine myelinolysis, centronuclear myopathy, cephalic disorder, cerebral aneurysm, cerebral arteriosclerosis, cerebral atrophy, cerebral gigantism, cerebral palsy, cerebral vasculitis, cervical spinal stenosis, Charcot-Marie-Tooth disease, Chiari malformation, chronic fatigue syndrome, chronic inflammatory demyelinating polyneuropathy (CIDP), chronic pain, Coffin-Lowry syndrome, complex regional pain syndrome, compression neuropathy, congenital facial diplegia, corticobasal degeneration, cranial arteritis, craniosynostosis, Creutzfeldt-Jakob disease, cumulative trauma disorder, Cushing's syndrome, cytomegalic inclusion body disease (CIBD), Dandy-Walker syndrome, Dawson disease, De Morsier's syndrome, Dejerine-Klumpke palsy, Dejerine-Sottas disease, delayed sleep phase syndrome, dementia, dermatomyositis, developmental dyspraxia, diabetic neuropathy, diffuse sclerosis, Dravet syndrome, dysautonomia, dyscalculia, dysgraphia, dyslexia, dystonia, empty sella syndrome, encephalitis, encephalocele, encephalotrigeminal angiomatosis, encopresis, epilepsy, Erb's palsy, erythromelalgia, essential tremor, Fabry's disease, Fahr's syndrome, familial spastic paralysis, febrile seizure, Fisher syndrome, Friedreich's ataxia, fibromyalgia, Foville's syndrome, Gaucher's disease, Gerstmann's syndrome, giant cell arteritis, giant cell inclusion disease, globoid cell leukodystrophy, gray matter heterotopia, Guillain-Barré syndrome, HTLV-1 associated myelopathy, Hallervorden-Spatz disease, hemifacial spasm, hereditary spastic paraplegia, heredopathia atactica polyneuritiformis, herpes zoster oticus, herpes zoster, Hirayama syndrome, holoprosencephaly, Huntington's disease, hydranencephaly, hydrocephalus, hypercortisolism, hypoxia, immune-mediated encephalomyelitis, inclusion body myositis, incontinentia pigmenti, infantile phytanic acid storage disease, infantile Refsum disease, infantile spasms, inflammatory myopathy, intracranial cyst, intracranial hypertension, Joubert syndrome, Karak syndrome, Kearns-Sayre syndrome, Kennedy disease, Kinsbourne syndrome, Klippel Feil syndrome, Krabbe disease, Kugelberg-Welander disease, kuru, Lafora disease, Lambert-Eaton myasthenic syndrome, Landau-Kleffner syndrome, lateral medullary (Wallenberg) syndrome, Leigh's disease, Lennox-Gastaut syndrome, Lesch-Nyhan syndrome, leukodystrophy, Lewy body dementia, lissencephaly, locked-in syndrome, Lou Gehrig's disease, lumbar disc disease, lumbar spinal stenosis, Lyme disease, Machado-Joseph disease (Spinocerebellar ataxia type 3), macrencephaly, macropsia, megalencephaly, Melkersson-Rosenthal syndrome, Meniere's disease, meningitis, Menkes disease, metachromatic leukodystrophy, microcephaly, micropsia, Miller Fisher syndrome, misophonia, mitochondrial myopathy, Mobius syndrome, monomelic amyotrophy, motor neuron disease, motor skills disorder, Moyamoya disease, mucopolysaccharidoses, multi-infarct dementia, multifocal motor neuropathy, multiple sclerosis, multiple system atrophy, muscular dystrophy, myalgic encephalomyelitis, myasthenia gravis, myelinoclastic diffuse sclerosis, myoclonic Encephalopathy of infants, myoclonus, myopathy, myotubular myopathy, myotonia congenital, narcolepsy, neurofibromatosis, neuroleptic malignant syndrome, lupus erythematosus, neuromyotonia, neuronal ceroid lipofuscinosis, Niemann-Pick disease, O'Sullivan-McLeod syndrome, occipital Neuralgia, occult Spinal Dysraphism Sequence, Ohtahara syndrome, olivopontocerebellar atrophy, opsoclonus myoclonus syndrome, optic neuritis, orthostatic hypotension, palinopsia, paresthesia, Parkinson's disease, paramyotonia Congenita, paraneoplastic diseases, paroxysmal attacks, Parry-Romberg syndrome, Pelizaeus-Merzbacher disease, periodic paralyses, peripheral neuropathy, photic sneeze reflex, phytanic acid storage disease, Pick's disease, polymicrogyria (PMG), polymyositis, porencephaly, post-polio syndrome, postherpetic neuralgia (PHN), postural hypotension, Prader-Willi syndrome, primary lateral sclerosis, prion diseases, progressive hemifacial atrophy, progressive multifocal leukoencephalopathy, progressive supranuclear palsy, pseudotumor cerebri, Ramsay Hunt syndrome type I, Ramsay Hunt syndrome type II, Ramsay Hunt syndrome type III, Rasmussen's encephalitis, reflex neurovascular dystrophy, Refsum disease, restless legs syndrome, retrovirus-associated myelopathy, Rett syndrome, Reye's syndrome, rhythmic movement disorder, Romberg syndrome, Saint Vitus dance, Sandhoff disease, schizophrenia, Schilder's disease, schizencephaly, sensory integration dysfunction, septo-optic dysplasia, Shy-Drager syndrome, Sjögren's syndrome, snatiation, Sotos syndrome, spasticity, spina bifida, spinal cord tumors, spinal muscular atrophy, spinocerebellar ataxia, Steele-Richardson-Olszewski syndrome, Stiff-person syndrome, stroke, Sturge-Weber syndrome, subacute sclerosing panencephalitis, subcortical arteriosclerotic encephalopathy, superficial siderosis, Sydenham's chorea, syncope, synesthesia, syringomyelia, tarsal tunnel syndrome, tardive dyskinesia, tardive dysphrenia, Tarlov cyst, Tay-Sachs disease, temporal arteritis, tetanus, tethered spinal cord syndrome, Thomsen disease, thoracic outlet syndrome, tic douloureux, Todd's paralysis, Tourette syndrome, toxic encephalopathy, transient ischemic attack, transmissible spongiform encephalopathies, transverse myelitis, tremor, trigeminal neuralgia, tropical spastic paraparesis, trypanosomiasis, tuberous sclerosis, ubisiosis, Von Hippel-Lindau disease (VHL), Viliuisk Encephalomyelitis (VE), Wallenberg's syndrome, Werdnig, Hoffman disease, west syndrome, Williams syndrome, Wilson's disease, and Zellweger syndrome.

The compounds and compositions may also be useful in the inhibition of the development of invasive cancer, tumor angiogenesis and metastasis.

In some embodiments, the pharmaceutical composition comprises a therapeutically effective amount of a compound of Formula (I), or a pharmaceutically acceptable salt thereof, and a pharmaceutically acceptable excipient.

In some embodiments, the disorder or disease is cancer.

In some embodiments, the disorder or disease is metastatic melanoma.

In some embodiments, the disorder or disease is diabetes.

In some embodiments, the disorder or disease is a neurological disorder.

In some embodiments, the disorder or disease is Alzheimer's disease.

In some embodiments, the patient is a human.

In some embodiments, the cancer is chosen from: hepatocellular carcinoma, colon cancer, breast cancer, pancreatic cancer, chronic myeloid leukemia (CML), chronic myelomonocytic leukemia, chronic lymphocytic leukemia (CLL), acute myeloid leukemia, acute lymphocytic leukemia, Hodgkin lymphoma, lymphoma, sarcoma, and ovarian cancer.

In some embodiments, the cancer is chosen from: lung cancer—non-small cell, lung cancer—small cell, multiple myeloma, nasopharyngeal cancer, neuroblastoma, osteosarcoma, penile cancer, pituitary tumors, prostate cancer, retinoblastoma, synovial sarcoma, rhabdomyosarcoma, salivary gland cancer, skin cancer—basal and squamous cell, skin cancer —melanoma, small intestine cancer, stomach (gastric) cancers, testicular cancer, thymus cancer, thyroid cancer, uterine sarcoma, vaginal cancer, vulvar cancer, laryngeal or hypopharyngeal cancer, kidney cancer, Kaposi sarcoma, gestational trophoblastic disease, gastrointestinal stromal tumor, gastrointestinal carcinoid tumor, gallbladder cancer, eye cancer (melanoma and lymphoma), Ewing tumor, esophagus cancer, endometrial cancer, colorectal cancer, cervical cancer, brain or spinal cord tumor, bone metastasis, bone cancer, bladder cancer, bile duct cancer, anal cancer and adrenal cortical cancer.

In some embodiments, the cancer is hepatocellular carcinoma; in some embodiments, the cancer is colon cancer; in some embodiments, the cancer is colorectal cancer; in some embodiments, the cancer is breast cancer; in some embodiments, the cancer is pancreatic cancer; in some embodiments, the cancer is chronic myeloid leukemia (CML); in some embodiments, the cancer is chronic myelomonocytic leukemia; in some embodiments, the cancer is chronic lymphocytic leukemia (CLL); in some embodiments, the cancer is acute myeloid leukemia; in some embodiments, the cancer is acute lymphocytic leukemia; in some embodiments, the cancer is Hodgkin lymphoma; in some embodiments, the cancer is lymphoma; in some embodiments, the cancer is sarcoma; in some embodiments, the cancer is ovarian cancer; in some embodiments, the cancer is lung cancer—non-small cell; in some embodiments, the cancer is lung cancer—small cell; in some embodiments, the cancer is multiple myeloma; in some embodiments, the cancer is nasopharyngeal cancer; in some embodiments, the cancer is neuroblastoma; in some embodiments, the cancer is osteosarcoma; in some embodiments, the cancer is penile cancer; in some embodiments, the cancer is pituitary tumors; in some embodiments, the cancer is prostate cancer; in some embodiments, the cancer is retinoblastoma; in some embodiments, the cancer is rhabdomyosarcoma; in some embodiments, the cancer is salivary gland cancer; in some embodiments, the cancer is skin cancer—basal and squamous cell; in some embodiments, the cancer is skin cancer—melanoma; in some embodiments, the cancer is small intestine cancer; in some embodiments, the cancer is stomach (gastric) cancers; in some embodiments, the cancer is testicular cancer; in some embodiments, the cancer is thymus cancer; in some embodiments, the cancer is thyroid cancer; in some embodiments, the cancer is uterine sarcoma; in some embodiments, the cancer is vaginal cancer; in some embodiments, the cancer is vulvar cancer; in some embodiments, the cancer is Wilms tumor; in some embodiments, the cancer is laryngeal or hypopharyngeal cancer; in some embodiments, the cancer is kidney cancer; in some embodiments, the cancer is Kaposi sarcoma; in some embodiments, the cancer is gestational trophoblastic disease; in some embodiments, the cancer is gastrointestinal stromal tumor; in some embodiments, the cancer is gastrointestinal carcinoid tumor; in some embodiments, the cancer is gallbladder cancer; in some embodiments, the cancer is eye cancer (melanoma and lymphoma); in some embodiments, the cancer is Ewing tumor; in some embodiments, the cancer is esophagus cancer; in some embodiments, the cancer is endometrial cancer; in some embodiments, the cancer is colorectal cancer; in some embodiments, the cancer is cervical cancer; in some embodiments, the cancer is brain or spinal cord tumor; in some embodiments, the cancer is bone metastasis; in some embodiments, the cancer is bone cancer; in some embodiments, the cancer is bladder cancer; in some embodiments, the cancer is bile duct cancer; in some embodiments, the cancer is anal cancer; and in some embodiments, the cancer is adrenal cortical cancer.

In some embodiments, the disorder or disease is a neurological condition, disorder, or disease, wherein the neurological disease is selected from: Alzheimer's disease, frontotemporal dementias, Parkinson's disease, Huntington's disease, progressive supranuclear palsy, corticobasal degeneration, multiple system atrophy, amyotrophic lateral sclerosis (ALS), inclusion body myositis, autism, degenerative myopathies.

In some embodiments, the disorder or disease is selected from the group consisting of: Alzheimer's Disease, Amyotrophic Lateral Sclerosis, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, and Stroke.

In some embodiments, a compound of Formula (I) inhibits DYRK1A.

In some embodiments, the method treats a disease or disorder mediated by kinase activity in a patient, the method comprises administering to the patient a therapeutically effective amount of a compound (or compounds) of Formula (I), or a pharmaceutically acceptable salt thereof.

In some embodiments, the disease or disorder comprises tumor growth, cell proliferation, or angiogenesis.

In some embodiments, the method inhibits the activity of a protein kinase receptor, the method comprises contacting the receptor with an effective amount of a compound (or compounds) of Formula (I), or a pharmaceutically acceptable salt thereof.

In some embodiments, the method treats a disease or disorder associated with aberrant cellular proliferation in a patient; the method comprises administering to the patient a therapeutically effective amount of a compound (or compounds) of Formula (I), or a pharmaceutically acceptable salt thereof.

In some embodiments, the method prevents or reduces abnormal cellular proliferation in a patient; the method comprises administering to the patient a therapeutically effective amount of a compound (or compounds) of Formula (I), or a pharmaceutically acceptable salt thereof.

In some embodiments, the method treats a disease or disorder associated with aberrant cellular proliferation in a patient, the method comprises administering to the patient a pharmaceutical composition comprising one or more of the compounds of claim 1 in combination with a pharmaceutically acceptable carrier and one or more other agents.

### Evaluation of Biological Activity

The biological activity of the compounds described herein can be tested using any suitable assay known to those of skill in the art. For example, the activity of a compound may be tested using one or more of the test methods outlined below.

For example, in vitro assays for DYRK1A biological activity may be used, e.g., regulation of microtubule-associated protein tau (MAPT/Tau) phosphorylation in neuronal cell lines such as the human SH-SY5Y neuroblastoma cell line. Assays for DYRK1A-regulated level of phosphorylation can include monitoring levels of basal pSer396 Tau, which can be measured, for example, by serial dilutions of a candidate inhibitor composition using a ten micromolar top concentration and detected by ELISA or Western Blotting. An exemplary assay for DYRK-1A-regulated phosphorylation uses the SH-SY5Y cells cultured in a 96 well plate format for a period of time sufficient to stabilize microtubules and Tau phosphorylation, usually at least 2 days, then treated with a 1/3 serial dilution of compounds overnight and lysed. The cell lysate is resolved by SDS PAGE, then transferred to nitrocellulose and probed with an antibody specific for pSer396 Tau. The chemiluminescence signal for HRP-linked antibodies used in western blotting is detected using a Carestream Image Station and blot densitometry for pSer396 and beta-actin are analyzed using ImageJ (NIH).

In a further example, the activity of a candidate compound can be measured by phosphoTau (Thr212) AlphaLISA by adding the lysate mentioned above onto total Tau-coated plates and detected with a specific pThr212Tau antibody. Colorimetric detection of AlphaLISA signal is performed by EnVision Multilabel Plate Reader (Perkin Elmer).

To further illustrate this disclosure, the following examples are included. The examples should not, of course, be construed as specifically limiting the disclosure. Variations of these examples within the scope of the claims are within the purview of one skilled in the art and are considered to fall within the scope of the disclosure as described and claimed herein. The reader will recognize that the skilled artisan, armed with the present disclosure, and skill in the art is able to prepare and use the disclosure without exhaustive examples.

### EXAMPLES

### Compound Preparation

The starting materials used in preparing the compounds of the disclosure are known, made by known methods, or are commercially available. It will be apparent to the skilled artisan that methods for preparing precursors and functionality related to the compounds claimed herein are generally described in the literature. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 7^{th} Ed., John Wiley & Sons (2013), Carey and Sundberg, Advanced Organic Chemistry 5^{th} Ed., Springer (2007), Comprehensive Organic Transformations: A Guide to Functional Group Transformations, 2^{nd} Ed., John Wiley & Sons (1999) (incorporated herein by reference in its entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in P. Wuts Greene's Protective Groups in Organic Synthesis, 5th Ed., John Wiley & Sons (2014), incorporated herein by reference in its entirety.

Trademarks used herein are examples only and reflect illustrative materials used at the time of the disclosure. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the disclosure.

(¹H) nuclear magnetic resonance spectra (NMR) were measured in the indicated solvents on a Bruker NMR spectrometer (Avance TM DRX300, 300 MHz for ¹H or Avance TM DRX500, 500 MHz for ¹H) or Varian NMR spectrometer (Mercury 400BB, 400 MHz for ¹H). Peak positions are expressed in parts per million (ppm) downfield from tetramethylsilane. The peak multiplicities are denoted as follows, s, singlet; d, doublet; t, triplet; q, quartet; ABq, AB quartet; quin, quintet; sex, sextet; sep, septet; non, nonet; dd, doublet of doublets; ddd, doublet of doublets of doublets; d/ABq, doublet of AB quartet; dt, doublet of triplets; td, triplet of doublets; dq, doublet of quartets; m, multiplet.

The following abbreviations have the indicated meanings:
Boc=tert-butyloxycarbonyl
BrettPhos=dicyclohexyl(2′,4′,6′-triisopropyl-3,6-dimethoxy-[1,1′-biphenyl]-2-yl) phosphine
BrettPhos Pd G3=[(2-di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate
^{t}BuXPhos-Pd-G3=methanesulfonato(2-di-^{t}butylphosphino-2′,4′,6′-tri-i-propyl-1,1′-biphenyl)(2′-amino-1,1′-biphenyl-2-yl)palladium (II)
brine=saturated aqueous sodium chloride
CBzCl=benzyl chloroformate
CDCl₃=deuterated chloroform
Cs₂CO₃=cesium carbonate
DCE=1,2-dichloroethane
DCM=dichloromethane
DIPEA=N,N-diisopropylethylamine
DME=1,2-dimethoxyethane, or glyme, or monoglyme
DMF=N,N-dimethylformamide
DMSO-d₆=deuterated dimethylsulfoxide
ESIMS=electron spray mass spectrometry
EtOAc=ethyl acetate
HATU=1-[bis(dimethylamino)methylene]-1H-1,2,3-triazolo[4,5-b]pyridinium 3-oxid hexafluorophosphate
HCl=hydrochloric acid
HOAc=acetic acid
ISCO=Teledyne ISCO, Inc brand CombiFlash® Rf 200
KHMDS=potassium bis(trimethylsilyl)amide
KOAc=potassium acetate
LCMS=liquid chromatography-mass spectrometry
LiHMDS=lithium bis(trimethylsilyl)amide
MCPBA=meta-chloroperoxybenzoic acid
MeCN=acetonitrile
MeOH=methanol
MeTHF=2-methyltetrahydrofuran
MgSO₄=magnesium sulfate
MTBE=methyl tert-butyl ether
MW=microwave irradiation
NaO^{t}Bu=Sodium tert-butoxide
NaHCO₃=sodium bicarbonate
NaBH(OAc)₃=Sodium triacetoxyborohydride
Na₂SO₄=sodium sulfate
NMR=nuclear magnetic resonance
ON=overnight
Pd/C=palladium on carbon
Pd(dppf)Cl₂=1,1′-bis(diphenylphosphino)ferrocene-palladium(II)dichloride
Pd(OAc)₂=palladium(II) acetate
PE=petroleum ether
Phosphazene base P2-Et=1-ethyl-2,2,4,4,4-pentakis(dimethylamino)-2λ⁵,4λ⁵-catenadi(phosphazene) or tetramethyl(tris(dimethylamino)phosphoranylidene) phosphorictriamid-Et-imin
QPhos=pentaphenyl(di-tert-butylphosphino)ferrocene
RP=reverse phase
r.t.=room temperature
SFC=supercritical fluid chromatography
TEA=triethylamine
TFA=trifluoroacetic acid
THF=tetrahydrofuran
TLC=thin layer chromatography
TMSOK=potassium trimethylsilanolate
XPhos=dicyclohexyl[2′,4′,6′-tris(propan-2-yl)[1,1′-biphenyl]-2-yl]phosphane

The following example schemes are provided for the guidance of the reader, and collectively represent an example method for making the compounds provided herein. Furthermore, other methods for preparing compounds of the disclosure will be readily apparent to the person of ordinary skill in the art in light of the following reaction schemes and examples. The skilled artisan is thoroughly equipped to prepare these compounds by those methods given the literature and this disclosure. The compound numberings used in the synthetic schemes depicted below are meant for those specific schemes only and should not be construed as or confused with same numberings in other sections of the application. Unless otherwise indicated, all variables are as defined above.

### General Procedures

Compounds of Formula I of the present disclosure can be prepared as depicted in Scheme 1.

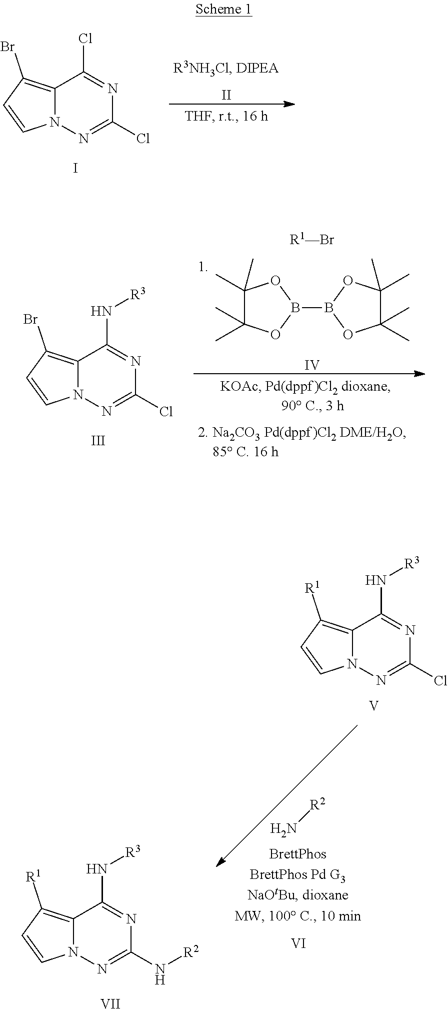

Scheme 1 describes a method for preparation of 4-alkylaminopyrrolo[2,1-f][1,2,4]triazine derivatives (VII) by first displacing the 4-chloride (I) with a variety of amines (II) to produce 2-chloro-5-bromo-4-aminopyrrolo[2,1-f][1,2,4]triazine III. Formation of a variety of boronic acid pinacol esters by reacting various bromides (IV) with bis(pinacolato)diboron followed by Suzuki coupling with bromide (III) produces 2-chloro-4-aminopyrrolo[2,1-f][1,2,4]triazine (V). The chloro is then displaced with a variety of amines (VI) to produce the final 4-aminopyrrolo[2,1-f][1,2,4]triazine (VII).

Compounds of Formula I of the present disclosure can also be prepared as depicted in Scheme 2.

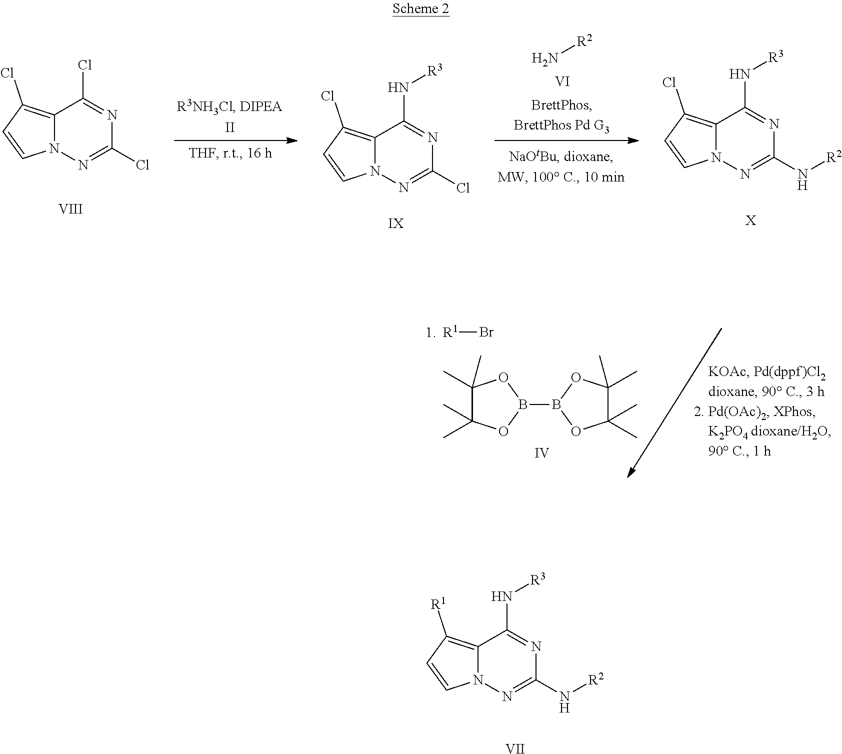

Scheme 2 describes a method for preparation of 4-aminopyrrolo[2,1-f][1,2,4]triazine derivatives (VII) by first displacing the 4-chloride (VIII) with a variety of amines (II) to produce 2,5-dichloro-4-aminopyrrolo[2,1-f][1,2,4]triazine IX. The 2-chloro is then displaced with a variety of amines (VI) to produce 5-chloro-4-aminopyrrolo[2,1-f][1,2,4]triazine (X). Formation of a variety of boronic acid pinacol esters by reacting various bromides (IV) with bis(pinacolato)diboron followed by Suzuki coupling with chloride (X) produces the final 4-aminopyrrolo[2,1-f][1,2,4]triazine (VII).

### Illustrative Compound Examples

Preparation of intermediate 5-bromo-2-chloro-N-methylpyrrolo[2,1-f][1,2,4]triazin-4-amine (XV) is depicted below in Scheme 3.

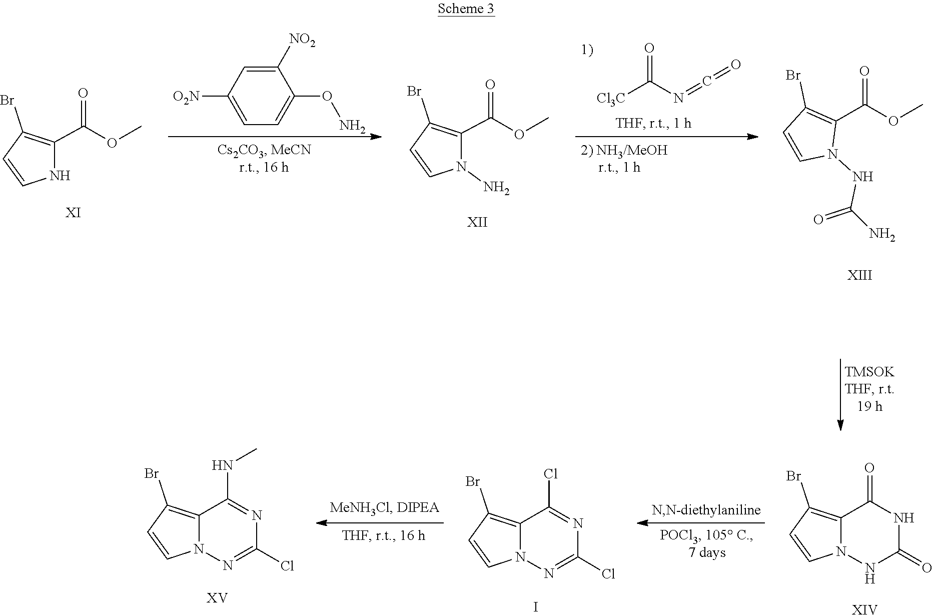

### Step 1

To a solution of methyl 3-bromo-1H-pyrrole-2-carboxylate (XI) (100 g, 490.15 mmol, 1 eq.) in MeCN (2 L) was added Cs₂CO₃ (255.52 g, 784.23 mmol, 1.6 eq.) in one portion at 20° C. (not exothermic). The reaction was stirred at room temperature for 5 h. A solution of O-(2,4-dinitrophenyl)hydroxylamine (146.40 g, 735.22 mmol, 1.5 eq.) in MeCN (2 L) was then added dropwise at 0˜5° C. under N₂. After addition, the reaction was warmed to room temperature for 16 h. The reaction mixture was filtered, and the filtrate was diluted with MTBE (2 L) and washed with brine (2 L×3). The above reaction was performed six times.

The combined organics of six batches were combined, dried over MgSO₄, filtered, and concentrated under vacuum to give the crude product. The crude product was purified by column chromatography on silica gel (0→50% DCM/PE, then 20% EtOAc/PE) to give methyl 1-amino-3-bromo-1H-pyrrole-2-carboxylate (XII) (380 g, 1.62 mol, 55.1% yield, 93.3% purity) as a yellow solid. ¹H NMR (400 MHz, DMSO-d₆) δ ppm 3.84 (3H, s), 6.24 (1H, d, J=2.8 Hz), 6.38 (2H, br s), 7.12 (1H, d, J=2.8 Hz).

### Steps 2-3

To a solution of methyl 1-amino-3-bromo-1H-pyrrole-2-carboxylate (XII) (190 g, 867.44 mmol, 1 eq.) in THF (1900 mL) was added dropwise 2,2,2-trichloroacetyl isocyanate (179.76 g, 954.18 mmol, 113.06 mL, 1.1 eq.) at 0˜5° C., then warmed to room temperature for 1 h. The reaction was then added dropwise to NH₃/MeOH (7 M, 1.24 L, 10 eq) at room temperature, stirred at room temperature for 1 h to give a yellow suspension. The above reaction was performed twice.

The two reactions were combined and concentrated under vacuum. The residue was triturated with MTBE (4 L) at room temperature for 30 min, filtered. The filter cake was washed with MTBE (500 mL×3), dried under vacuum to give methyl 3-bromo-1-ureido-1H-pyrrole-2-carboxylate (XIII) (402 g, 1.53 mol, 88.2% yield, 99.78% purity) as an off-white solid.

¹H NMR (400 MHz, DMSO-d₆) δ ppm 3.75 (3H, s), 6.27 (2H, br s), 6.28 (1H, d, J=2.8 Hz), 7.10 (1H, d, J=2.8 Hz), 9.27 (1H, br s); ESIMS found for C₇H₈BrN₃O₃ m/z 262.1 (⁷⁹BrM+H).

### Step 4

To a solution of methyl 3-bromo-1-ureido-1H-pyrrole-2-carboxylate (XIII) (67 g, 255.67 mmol, 1 eq.) in THF (4 L) was added TMSOK (65.60 g, 511.33 mmol, 2 eq.) in portions at 0˜5° C. under N₂. After stirring for 5 min, the reaction became a thick slurry. The reaction mixture was stirred vigorously at room temperature for 16 h. LCMS showed ˜25.8% of starting material remained. An additional portion of TMSOK (16.40 g, 127.83 mmol, 0.5 eq.) was added at room temperature and stirred for 3 h. LCMS showed the reaction was complete. The reaction was concentrated. The residue was diluted with H₂O (1.6 L), cooled with ice-bath, added dropwise HCl (4 M, 160 mL) to pH 2˜3, and filtered. The filter cake was washed with H₂O (600 mL×3). The above reaction was performed six times.

The filter cakes of six reactions were combined, triturated with H₂O (6 L) at room temperature, for 2 h and then filtered. The filter cake was dried in a vacuum drying oven at 60° C. for 24 h to give 5-bromopyrrolo[2,1-f][1,2,4]triazine-2,4(1H,3H)-dione (XIV) (320 g, 1.28 mol, 83.4% yield, 91.95% purity) as a white solid. ¹H NMR (400 MHz, DMSO-d₆) δ ppm 6.49 (1H, d, J=2.8 Hz), 7.17 (1H, d, J=2.8 Hz), 11.30 (1H, br s); ESIMS found for C₆H₄BrN₃O₂ m/z 232.0 (⁸¹BrM+H).

### Step 5

To a solution of POCl₃ (1500 mL) was added 5-bromopyrrolo[2,1-f][1,2,4]triazine-2,4(1H,3H)-dione (XIV) (180 g, 782.55 mmol, 1 eq.) in portions at room temperature. N,N-Diethylaniline (291.95 g, 1.96 mol, 312.92 mL, 2.5 eq.) was then added dropwise at room temperature (a little exothermic). After addition, the reaction was heated to 105° C. for 48 h. LCMS showed the starting material was consumed but the intermediate remained, ˜4.3% of desired product was formed. The reaction was cooled to 90° C., added an additional POCl₃ (500 mL) in one portion, then heated to reflux gently for 24 h. LCMS showed most of intermediate remained, ˜11.9% of desired product was formed. The reaction was heated to reflux gently for another 4 days. LCMS showed ˜7.4% of intermediate remained, ˜55.5% of desired product was formed. The reaction was cooled to 40° C. and distilled under reduced pressure to remove most of POCl₃. The residue was diluted with MeTHF (5 L), poured into ice-H₂O (2 L), the added brine (1 L), and separated. The aqueous layer was extracted with MeTHF (1.5 L×2). The combined organic were washed with brine (1.5 L×2), dried over MgSO₄, filtered, and concentrated under vacuum to give the crude product. The crude product was purified by column chromatography on silica gel (4 kg, 100˜200 mesh, 0→20% DCM/PE) and triturated with n-heptane (500 mL) at room temperature for 3 h, then filtered to afford 5-bromo-2,4-dichloropyrrolo[2,1-f][1,2,4]triazine (I) (150 g, 556.14 mmol, 71.1% yield, 98.96% purity) as a yellow solid. ¹H NMR (400 MHz, CDCl₃) δ ppm 7.02 (1H, d, J=2.4 Hz), 7.79 (1H, d, J=2.4 Hz); ESIMS found for C₆H₂BrCl₂N₃ m/z 266.1 (⁷⁹BrM+H).

### Step 6

To a stirred solution of 5-bromo-2,4-dichloropyrrolo[2,1-f][1,2,4]triazine (I) (10 g, 37.47 mmol) in THF (250 mL) was added methylammonium chloride (6.33 g, 93.75 mmol) and DIPEA (22.9 mL, 131.47 mmol). The mixture was stirred at room temperature for 16 h. The reaction mixture was quenched with water, extracted in EtOAc, washed with brine, and dried over anhydrous Na₂SO₄. The solvent was evaporated on rotavapor, and the crude mixture was purified on ISCO (0→10% MeOH/CHCl₃) to obtain 5-bromo-2-chloro-N-methylpyrrolo[2,1-f][1,2,4]triazin-4-amine (XV) (8.9 g, 34.033 mmol, 90.8% yield) as a white solid. ESIMS found for C₇H₆BrClN₄ m/z 261.0 (M+H).

The following intermediates were prepared in accordance with the procedure described in the above Scheme 3.

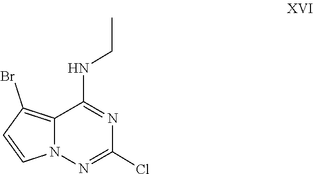

5-Bromo-2-chloro-N-ethylpyrrolo[2,1-f][1,2,4]triazin-4-amine (XVI): Beige solid (503.6 mg, 1.831 mmol, 97.6% yield). ESIMS found C₈H₈BrClN₄ m/z 275.0 (M+H).

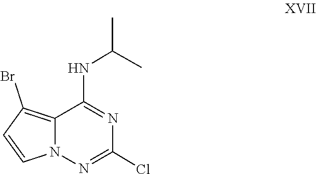

5-Bromo-2-chloro-N-isopropylpyrrolo[2,1-f][1,2,4]triazin-4-amine (XVII): White solid (453.0 g, 1.564 mmol, 83.5% yield). ESIMS found C₉H₁₀BrClN₄ m/z 289.0 (M+H).

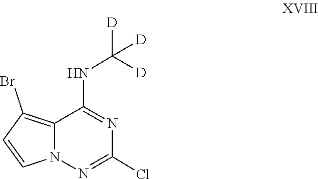 [SMILES: [2H]C([2H])([2H])Nc1nc(Cl)nn2ccc(Br)c12]

5-Bromo-2-chloro-N-(methyl-d₃)pyrrolo[2,1-f][1,2,4]triazin-4-amine (XVIII): Orange solid (647 mg, 2.446 mmol, 21.8% yield). ESIMS found C₇H₃D₃BrClN₄ m/z 264.0 (M+H).

Preparation of intermediate 5-bromo-2-chloropyrrolo[2,1-f][1,2,4]triazin-4-amine (XIX) is depicted below in Scheme 4.

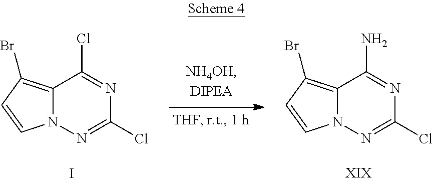

### Step 1

To a stirred solution of ammonium hydroxide (4.7 mL, 37.47 mmol) in THF (10 mL) was added 5-bromo-2,4-dichloropyrrolo[2,1-f][1,2,4]triazine (I) (1 g, 3.75 mmol) and the mixture was stirred at room temperature for 1 h. The reaction mixture was extracted in DCM, washed with brine, and dried over anhydrous Na₂SO₄. The solvent was evaporated on rotavapor and dried under high vacuo to obtain 5-bromo-2-chloropyrrolo[2,1-f][1,2,4]triazin-4-amine (XIX) (900 mg, 3.637 mmol, 97.1% yield) as an off-white solid. ESIMS found for C₆H₄BrClN₄ m/z 246.95 (M+H).

The following intermediate was prepared in accordance with the procedure described in the above Scheme 4.

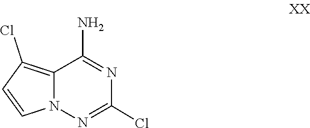 [SMILES: Nc1nc(Cl)nn2ccc(Cl)c12]

2,5-Dichloropyrrolo[2,1-f][1,2,4]triazin-4-amine (XX): Pale-orange solid (308 mg, 1.517 mmol, 16.9% yield). ESIMS found C₆H₄C₁₂N₄ m/z 203.0 (M+H).

Preparation of intermediate 2,5-dichloro-N-methylpyrrolo[2,1-f][1,2,4]triazin-4-amine (XXI) is depicted below in Scheme 5.

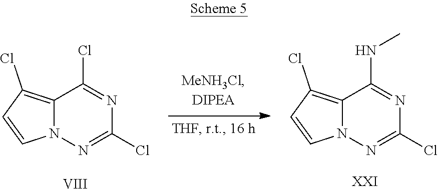

### Step 1

To a stirred solution of 2,4,5-trichloropyrrolo[2,1-f][1,2,4]triazine (commercially available from PharmaBlock Sciences Inc.) (VIII) (2 g, 8.99 mmol) in THF (50 mL) were added methylammonium chloride (1.51 g, 22.36 mmol) and DIPEA (5.48 mL, 31.46 mmol) at −78° C. and the mixture was stirred at room temperature for 16 h. The reaction mixture was quenched with saturated NaHCO₃ solution, extracted with DCM, washed with brine, and dried over anhydrous Na₂SO₄. The solvent was evaporated on rotavapor and dried under high vacuo to obtain 2,5-dichloro-N-methylpyrrolo[2,1-f][1,2,4]triazin-4-amine (XXI) (1.92 g, 8.846 mmol, 98.4% yield) as a yellow solid. ESIMS found for C₇H₆Cl₂N₄ m/z 217.0 (M+H).

Preparation of intermediates 5-bromo-2-chloro-6-fluoro-4-methoxypyrrolo [2,1-f][1,2,4]triazine (XXXI) and 5-bromo-2-chloro-6-fluoro-N-methylpyrrolo[2,1-f][1,2,4]triazin-4-amine (XXXII) are depicted below in Scheme 6.

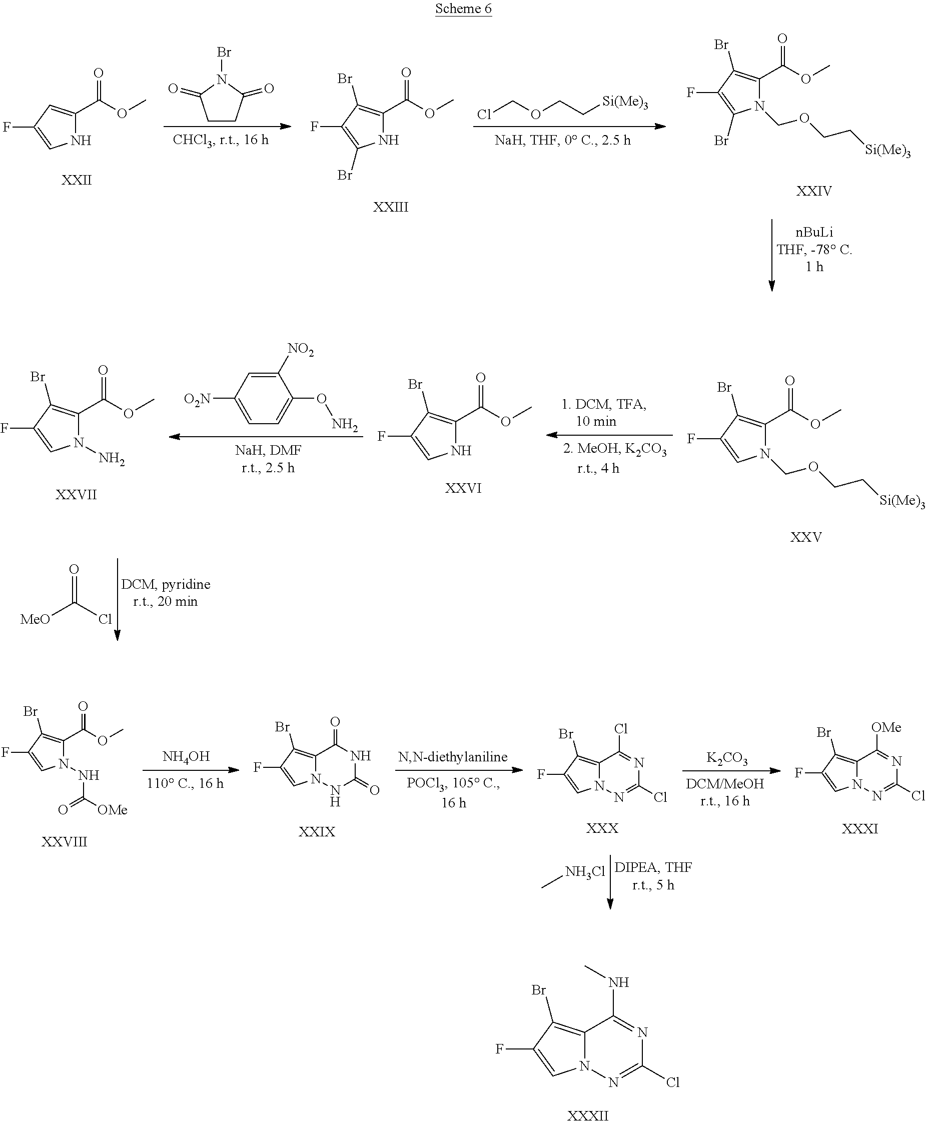

### Step 1

To a solution of methyl 4-fluoro-1H-pyrrole-2-carboxylate (XXII) (commercially available from PharmaBlock (USA), Inc.) (1 g, 6.99 mmol) in CHCl₃ (9 mL) at 0° C. was added N-bromosuccinimide (2.64 g, 14.68 mmol). The reaction was stirred at room temperature for 16 h. The reaction mixture was quenched with 10% sodium thiosulfate (50 mL) and extracted with CHC₃ (3×30 mL). The organic layers were combined and washed with aqueous saturated NaCl (50 mL) and evaporated under reduced pressure to produce methyl 3,5-dibromo-4-fluoro-1H-pyrrole-2-carboxylate (XXIII) (2.1 g, 6.979 mmol, 99.9% yield) as a beige solid. The product was used as is without further purification. ESIMS found for C₆H₄Br₂FNO₂ m/z 299.9 (M+H).

### Step 2

To a solution of methyl 3,5-dibromo-4-fluoro-1H-pyrrole-2-carboxylate (XXIII) (2.1 g, 6.98 mmol) in dry THF (25 mL) at 0° C. under N₂ was added slowly NaH (430 mg, 10.63 mmol) in small portions. The suspension was stirred at 0° C. for 30 min. To the suspension was added (2-(chloromethoxy)ethyl)trimethylsilane (1.9 mL, 10.74 mmol) at 0° C. under N₂. The reaction mixture was stirred at 0° C. for 2 h before quenching with ice-water (20 mL) and extracted with EtOAc (3×30 mL). The EtOAc layer was evaporated under reduced pressure and purified by silica gel column chromatography (100% hexanes) to produce methyl 3,5-dibromo-4-fluoro-1-((2-(trimethylsilyl)ethoxy)methyl)-1H-pyrrole-2-carboxylate (XXIV) (2.76 g, 6.401 mmol, 91.7% yield) as an amber liquid. ¹H NMR (499 MHz, DMSO-d₆) δ ppm −0.07 (9H, s), 0.81 (2H, t, J=7.80 Hz), 3.50 (2H, t, J=7.80 Hz), 3.82 (3H, s), 5.69 (2H, s).

### Step 3

To solution of methyl 3,5-dibromo-4-fluoro-1-((2-(trimethylsilyl)ethoxy) methyl)-1H-pyrrole-2-carboxylate (XXIV) (2.76 g, 6.4 mmol) in dry THF (60 mL) at −78° C. under N₂ was added nBuLi (2.4 mL, 6. mmol). The reaction mixture was stirred at −78° C. for 1 h. The mixture was carefully quenched with ice water (20 mL), warmed to room temperature, and extracted with EtOAc (3×20 mL). The organics were dried over anhydrous Na₂SO₄, filtered, and evaporated onto Celite® and purified by silica gel column chromatography (0→6% EtOAc/hexanes) to produce methyl 3-bromo-4-fluoro-1-((2-(trimethylsilyl)ethoxy)methyl)-1H-pyrrole-2-carboxylate (XXV) (760 mg, 2.157 mmol, 33.7% yield) as a clear oil. ¹H NMR (500 MHz, DMSO-d₆) δ ppm −0.06 (9H, s), 0.80 (2H, t, J=7.96 Hz), 3.41-3.50 (2H, m), 3.80 (3H, s), 5.56 (2H, s), 7.52 (1H, d, J=3.29 Hz).

### Step 4

To a stirred solution of methyl 3-bromo-4-fluoro-1-((2-(trimethylsilyl) ethoxy)methyl)-1H-pyrrole-2-carboxylate (XXV) (881 mg, 2.5 mmol) in DCM (23 mL) was added TFA (23 mL) and the mixture was stirred at room temperature for 10 min. The solvents were concentrated, the residue taken in a mixture of MeOH (12 mL)/water (6 mL). To this mixture was added K₂CO₃ (1.75 g, 12.66 mmol) and stirred at room temperature for 4 h. Water (10 mL) was added, and the solution was neutralized with 1H HCl and washed with CHCl₃ (3×10 mL). The organic layer was stripped onto Celite® and purified by silica gel column chromatography (0→5% 7 N NH₃ in MeOH/CHCl₃) to produce methyl 3-bromo-4-fluoro-1H-pyrrole-2-carboxylate (XXVI) (450 mg, 2.027 mmol, 81.0% yield) as an off white solid. ¹H NMR (499 MHz, DMSO-d₆) δ ppm 3.80 (3H, s), 7.15 (1H, d, J=3.56 Hz), 12.29 (1H, br s); ESIMS found for C₆H₅BrFNO₂ m/z 222.0 (M+H).

### Step 5

To a solution of methyl 3-bromo-4-fluoro-1H-pyrrole-2-carboxylate (XXVI) (500 mg, 2.25 mmol) in dry DMF (7 mL) at 0° C. was slowly added NaH (120 mg, 2.93 mmol). After 30 min, O-(2,4-dinitrophenyl)hydroxylamine (490 mg, 2.48 mmol)) was added in one portion and the reaction was stirred in the ice bath for 1 h and at room temperature for 1 h. LC/MS showed the reaction was incomplete so an additional portion of O-(2,4-dinitrophenyl)hydroxylamine (490 mg, 2.48 mmol) was added and stirred at room temperature for another 15 min. The reaction was diluted with water (100 mL) and extracted with EtOAc (3×75 mL). The combined extracts were stripped onto Celite® and purified by silica gel column chromatography (100% CHCl₃) to produce methyl 1-amino-3-bromo-4-fluoro-1H-pyrrole-2-carboxylate (XXVII) (501 mg, 2.114 mmol, 93.9% yield) as an off-white solid. ¹H NMR (499 MHz, DMSO-d₆) δ ppm 3.80 (3H, s), 6.36 (2H, s), 7.25 (1H, d, J=3.29 Hz); ESIMS found for C₆H₆BrFN₂O₂ m/z 237.0 (M+H).

### Step 6

To a solution of methyl 1-amino-3-bromo-4-fluoro-1H-pyrrole-2-carboxylate (XXVII) (500 mg, 2.11 mmol) in DCM (10 mL) was added pyridine (210 μL, 2.6 mmol) and methyl carbonochloridate (180 μL, 2.33 mmol) dropwise. The reaction was stirred at room temperature for 20 min. DCM (100 mL) was added and washed with 1 N HCl (2×50 mL), stripped onto Celite® and purified by silica gel column chromatography (0→100% EtOAc/hexanes) to produce methyl 3-bromo-4-fluoro-1-((methoxycarbonyl) amino)-1H-pyrrole-2-carboxylate (XXVIII) (560 mg, 1.898 mmol, 89.8% yield) as a clear oil. ¹H NMR (499 MHz, DMSO-d₆) δ ppm 3.69 (3H, br s), 3.76 (3H, s), 7.48 (1H, br d, J=1.92 Hz), 10.76 (1H, br s); ESIMS found for C₈H₈BrFN₂O₄ m/z 295.0 (M+H).

### Step 7

To NH₄OH (35 mL, 263.7 mmol) in a sealed tube was added methyl 3-bromo-4-fluoro-1-((methoxycarbonyl)amino)-1H-pyrrole-2-carboxylate (XXVIII) (560 mg, 1.9 mmol). The reaction was sealed and heated to 110° C. for 16 h. The solvent was removed under vacuum and the residue was dissolved in MeCN. The solvent was stripped (3× to remove water) and placed under high vacuum for 1 h, to produce 5-bromo-6-fluoropyrrolo[2,1-f][1,2,4]triazine-2,4(1H,3H)-dione (XXIX) (440 mg, 1.774 mmol, 93.5% yield) as a light brown solid. The product was used as is without further purification. ¹H NMR (499 MHz, DMSO-d₆) δ ppm 7.04 (1H, d, J=3.29 Hz), 7.13 (1H, br s), 9.58 (1H, br s); ESIMS found for C₆H₃BrFN₃O₂ m/z 247.9 (M+H).

### Step 8

To a solution of neat POCl₃ (3 mL, 32.19 mmol) was added 5-bromo-6-fluoropyrrolo[2,1-f][1,2,4]triazine-2,4(1H,3H)-dione (XXIX) (440 mg, 1.77 mmol) in portions at room temperature. N,N-Diethylaniline (0.7 mL, 4.4 mmol) was then added dropwise at room temperature. The reaction was heated at 105° C. for 16 h. The solvent was removed under vacuum before adding water (20 mL) and extracting with EtOAc. The organic layer was stripped onto Celite® and purified by silica gel column chromatography (0→100% EtOAc/hexanes) to produce 5-bromo-2,4-dichloro-6-fluoropyrrolo[2,1-f][1,2,4]triazine (XXX) (175 mg, 0.614 mmol, 34.6% yield) as an off-white solid. ¹H NMR (499 MHz, DMSO-d₆) δ ppm 7.92 (1H, d, J=3.29 Hz); ESIMS found for C₆HBrCl₂FN₃ m/z 283.9 (M+H).

### Step 9

To a solution of 5-bromo-2,4-dichloro-6-fluoropyrrolo[2,1-f][1,2,4]triazine (XXX) (175 mg, 0.61 mmol) in MeOH (664 μL) and DCM (2 mL) was added K₂CO₃ (254.1 mg, 1.84 mmol). The flask was capped and the reaction and stirred at room temperature for 16 h. Water (50 mL) was added and extracted with DCM (3×50 mL). The organic layers were separated and washed with water (2×50 mL) and brine (50 mL). The solvent was removed under vacuum to produce 5-bromo-2-chloro-6-fluoro-4-methoxypyrrolo[2,1-f][1,2,4]triazine (XXXI) (163 mg, 0.581 mmol, 94.6% yield) as a beige solid. ¹H NMR (500 MHz, DMSO-d₆) δ ppm 4.12 (3H, s), 8.34 (1H, d, J=3.29 Hz); ESIMS found for C₇H₄BrClFN₃O m/z 279.95 (M+H).

### Step 10

To a solution of 5-bromo-2,4-dichloro-6-fluoropyrrolo[2,1-f][1,2,4]triazine (XXX) (4.0 g, 14.04 mmol) in THF (100 mL) were added methylammonium chloride (1.04 g, 15.4 mmol) and DIPEA (8.4 mL, 48.23 mmol) at 0° C. The reaction was stirred vigorously for 5 h at room temperature. The solvent was evaporated on rotavapor, and the reaction mixture was extracted with DCM and washed with aqueous NH₄Cl solution and then reduced in vacuo. The crude mixture was purified on ISCO (0→40% EtOAc in hexanes). Appropriate fractions were collected and concentrated under reduced pressure and dried under high vacuo to obtain 5-bromo-2-chloro-6-fluoro-N-methylpyrrolo[2,1-f][1,2,4]triazin-4-amine (XXXII) (3.55 g, 12.701 mmol, 90.5% yield) as a fluffy yellow solid. ESIMS found for C₇H₅BrClFN₄ m/z 278.95 (M+H).

The following intermediates were prepared in accordance with the procedure described in the above Scheme 6.

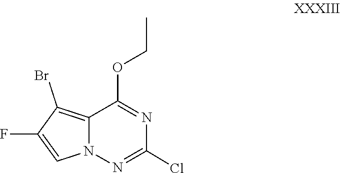

5-Bromo-2-chloro-4-ethoxy-6-fluoropyrrolo[2,1-f][1,2,4]triazine (XXXIII): Pale-yellow solid (264 mg, 0.896 mmol, 85.1% yield). ESIMS found C₈H₆BrClFN₃O m/z 293.95 (M+H).

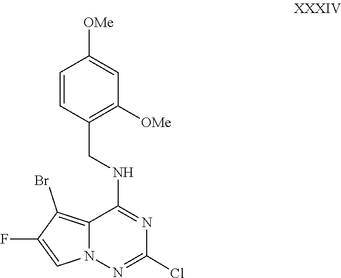 [SMILES: COc1ccc(CNc2nc(Cl)nn3cc(F)c(Br)c23)c(OC)c1]

5-Bromo-2-chloro-N-(2,4-dimethoxybenzyl)-6-fluoropyrrolo[2,1-f][1,2,4]triazin-4-amine (XXXIV): Beige solid (670 mg, 1.612 mmol, 91.8% yield). ESIMS found C₁₅H₁₃BrClFN₄O₂ m/z 415.0 (M+H).

Preparation of intermediate 5-chloro-3-(2-fluoroethyl)-3H-imidazo[4,5-b]pyridine (XXXVI) is depicted below in Scheme 7.

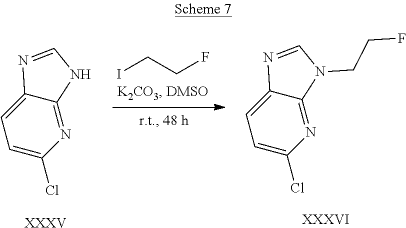

### Step 1

A mixture of 5-chloro-3H-imidazo[4,5-b]pyridine (XXXV) (1 g, 6.51 mmol), K₂CO₃ (1.8 g, 13.02 mmol) and 1-fluoro-2-iodoethane (1.7 g, 9.77 mmol) in DMSO (6 mL) was stirred at room temperature for 48 h. The reaction mixture was poured into a solution of EtOAc and water. The organic layer was separated, and the aqueous layer was extracted with EtOAc (3×). The combined organic layers were washed with brine, dried over MgSO₄, filtered, and concentrated. The crude product was purified via column chromatography (80 g of silica gel) (50→100% EtOAc/hexanes) to obtain 5-chloro-3-(2-fluoroethyl)-3H-imidazo[4,5-b]pyridine (XXXVI) (470 mg, 2.355 mmol, 36.2% yield) as a beige solid which was used for next step without further purification. ESIMS found C₈H₇ClFN₃ m/z 200.05 (M+H).

The following intermediates were prepared in accordance with the procedure described in the above Scheme 7.

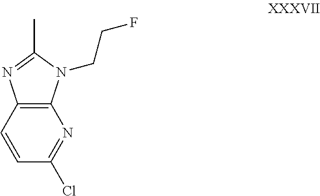

5-Chloro-3-(2-fluoroethyl)-2-methyl-3H-imidazo[4,5-b]pyridine (XXXVII): Beige solid (220 mg, 1.030 mmol, 57.5% yield). ESIMS found C₉H₉ClFN₃ m/z 214.05 (M+H).

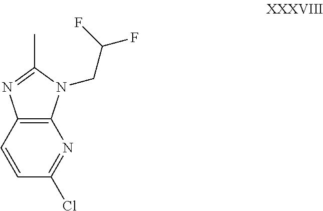

5-Chloro-3-(2,2-difluoroethyl)-2-methyl-3H-imidazo[4,5-b]pyridine (XXXVIII): Beige solid (830 mg, 3.583 mmol, 60.1% yield). ESIMS found C₉H₈ClF₂N₃ m/z 232.0 (M+H).

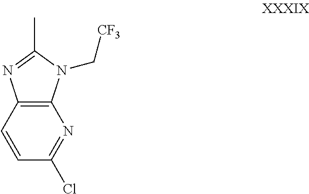 [SMILES: Cc1nc2ccc(Cl)nc2n1CC(F)(F)F]

5-Chloro-2-methyl-3-(2,2,2-trifluoroethyl)-3H-imidazo[4,5-b]pyridine (XXXIX): Beige solid (372 mg, 1.490 mmol, 50.0% yield). ESIMS found C₉H₇ClF₃N₃ m/z 250.0 (M+H).

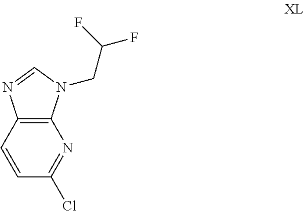 [SMILES: FC(F)Cn1cnc2ccc(Cl)nc21]

5-Chloro-3-(2,2-difluoroethyl)-3H-imidazo[4,5-b]pyridine (XL): White solid (253 mg, 1.163 mmol, 20.5% yield). ESIMS found C₈H₆ClF₂N₃ m/z 218.0 (M+H).

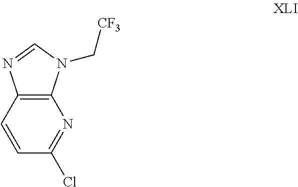 [SMILES: FC(F)(F)Cn1cnc2ccc(Cl)nc21]

5-Chloro-3-(2,2,2-trifluoroethyl)-3H-imidazo[4,5-b]pyridine (XLI): Light yellow solid (335 mg, 1.422 mmol, 21.8% yield). ESIMS found C₈H₅ClF₃N₃ m/z 236.0 (M+H).

Preparation of intermediate 5-chloro-3-(2,2-difluoroethyl)-3H-[1,2,3]triazolo[4,5-b]pyridine (XLVI) is depicted below in Scheme 8.

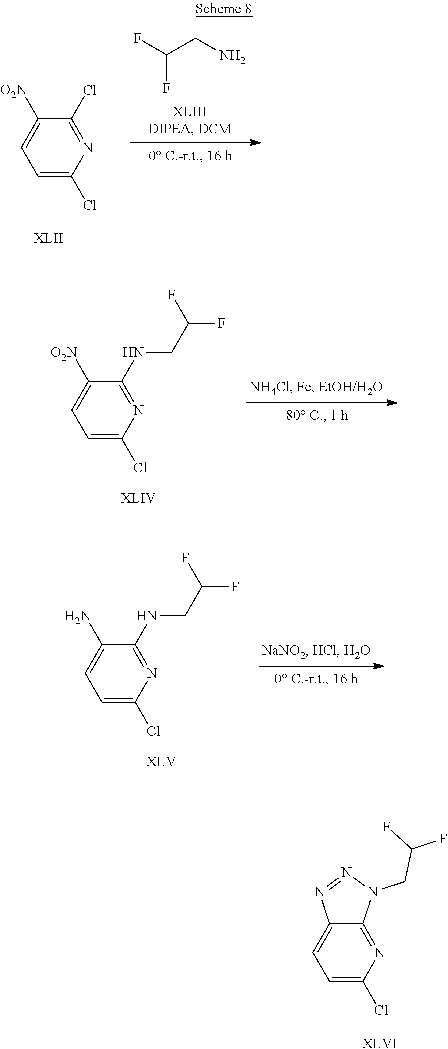

### Step 1

To a solution of 2,6-dichloro-3-nitropyridine (XLII) (14.0 g, 73.30 mmol) in DCM (500.0 mL) cooled to 0° C. was added 2,2-difluoroethan-1-amine (XLIII) (8.91 g, 109.95 mmol) at 0° C. DIPEA (18.91 g, 146.60 mmol) was then added, and the reaction was warmed to room temperature for 16 h. The reaction mixture was extracted with DCM (500 mL×2). The combined organics were washed with brine (500 mL×3), dried over anhydrous Na₂SO₄, filtered, and concentrated to give the crude product. The crude was purified by silica gel column chromatography (PE/EtOAc=10:1→5:1) to give 6-chloro-N-(2,2-difluoroethyl)-3-nitropyridin-2-amine (XLIV) (12.0 g, 50.507 mmol, 68.9%) as a yellow solid. ¹H NMR (400 MHz, DMSO-d₆) δ 4.01-3.93 (m, 2H), 6.43-6.03 (m, 1H), 6.95-6.79 (m, 1H), 8.54-8.40 (m, 1H), 8.81 (t, J=5.4 Hz, 1H).

### Step 2

6-Chloro-N-(2,2-difluoroethyl)-3-nitropyridin-2-amine (XLIV) (12.0 g, 50.51 mmol) was added to a mixture of NH₄Cl (7.37 g, 139.00 mmol) in EtOH/H₂O (300/100 mL, 3:1 ratio). The resulting mixture was sealed and heated at 80° C. Fe (12.97 g, 231.66 mmol) was then added at 80° C., sealed and heated at 80° C. for 1 h. The reaction mixture was cooled to room temperature, filtered, and diluted with EtOAc (300 mL×2). The solution was washed with water (300 mL×2), and the aqueous portion was extracted with EtOAc (300 mL×2). The combined organic extracts were washed with brine (300 mL×2), dried over anhydrous Na₂SO₄, filtrated, and concentrated in vacuo to afford the crude product. The crude product was purified by silica gel column chromatography (PE/EtOAc=10:1→3:1) to afford the 6-chloro-N²-(2,2-difluoroethyl)pyridine-2,3-diamine (XLV) (10.0 g, 48.167 mmol, 95.4% yield) as a purple solid.

¹H NMR (400 MHz, DMSO-d₆) δ 6.76 (d, J=7.8 Hz, 1H), 3.76-3.66 (m, 2H), 4.93 (s, 2H), 6.11 (tt, J=56.6, 4.2 Hz, 1H), 6.45 (d, J=7.8 Hz, 1H); ESIMS found for C₇H₈ClF₂N₃ m/z 208.1 (M+H).

### Step 3

To a solution of 6-chloro-N²-(2,2-difluoroethyl)pyridine-2,3-diamine (XLV) (10.0 g, 48.17 mmol) in HCl (360 mL) at 0° C. was added NaNO₂ (4.00 g, 57.97 mmol) in H₂O (55 mL). The mixture was stirred at room temperature at 16 h under N₂. The reaction mixture was adjusted to pH=9 with 4 M NaOH and extracted with EtOAc (300 mL×3). The combined organic layers were washed with brine and dried over anhydrous Na₂SO₄, filtered, and concentrated to give the crude product. The crude product was purified by silica gel column chromatography (PE/EtOAc=10:1→1:1) to yield 5-chloro-3-(2,2-difluoroethyl)-3H-[1,2,3]triazolo[4,5-b]pyridine (XLVI) (6.2 g, 28.364 mmol, 58.9% yield) as a yellow solid. ¹H NMR (400 MHz, DMSO-d₆) δ 5.25 (td, J=16.0, 3.0 Hz, 2H), 6.85-6.41 (m, 1H), 7.65 (d, J=8.6 Hz, 1H), 8.72 (d, J=8.6 Hz, 1H); ESIMS found for C₇H₅ClF₂N₄ m/z 219.0 (M+H).

The following intermediates were prepared in accordance with the procedure described in the above Scheme 8.

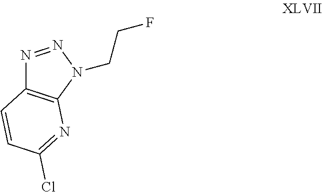 [SMILES: FCCn1nnc2ccc(Cl)nc21]

5-Chloro-3-(2-fluoroethyl)-3H-[1,2,3]triazolo[4,5-b]pyridine (XLVII): Yellow solid (2.7 g, 13.460 mmol, 39.3% yield). ¹H NMR (400 MHz, DMSO-d₆) δ 5.02-4.90 (m, 2H), 5.10-5.03 (m, 2H), 7.62 (d, J=8.6 Hz, 1H), 8.68 (d, J=8.6 Hz, 1H); ESIMS found for C₇H₆ClFN₄ m/z 201.1 (M+H).

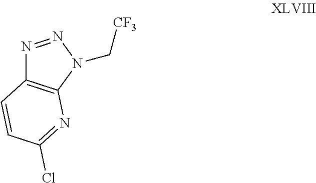 [SMILES: FC(F)(F)Cn1nnc2ccc(Cl)nc21]

5-Chloro-3-(2,2,2-trifluoroethyl)-3H-[1,2,3]triazolo[4,5-b]pyridine (XLVIII): Yellow solid (3.1 g, 13.103 mmol, 42.2%). ¹H NMR (400 MHz, DMSO-d₆) δ 5.83 (q, J=9.2 Hz, 2H), 7.70 (d, J=8.6 Hz, 1H), 8.76 (d, J=8.6 Hz, 1H); ESIMS found for C₇H₄ClF₃N₄ m/z 237.0 (M+H).

Preparation of intermediate 3-(2,2-difluoroethyl)-5-(4,4,5,5-tetramethyl-1,3,2-dioxaborolan-2-yl)-3H-[1,2,3]triazolo[4,5-b]pyridine (XLIX) is depicted below in Scheme 9.

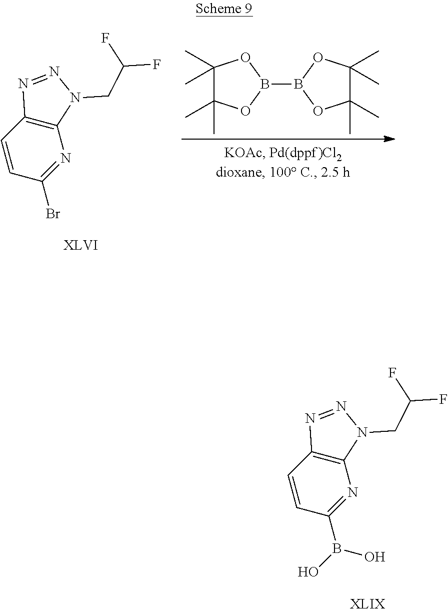

### Step 1

5-Chloro-3-(2,2-difluoroethyl)triazolo[4,5-b]pyridine (XLVI) (5.43 g, 24.84 mmol), bis(pinacolato)diboron (8.16 g, 32.13 mmol), Pd(dppf)Cl₂ (1.02 g, 1.25 mmol) and KOAc (7.35 g, 74.89 mmol) were suspended in dry 1,4-dioxane (100 mL). The reaction was sonicated and degassed with Ar for 5 minutes. The reaction was stirred at 90° C. for 2.5 h. Celite® was added and the reaction mixture was reduced in vacuo and then purified by RP column chromatography (C18Aq with loading column to trap Pd) (0→28% MeCN/H₂O (0.1% formic acid)). Appropriate fractions were pooled and reduced in vacuo to ˜100 mL and the resulting precipitate was filtered to give [3-(2,2-difluoroethyl)triazolo[4,5-b]pyridin-5-yl]boronic acid (XLIX) (4.207 g, 18.455 mmol, 74.3% yield) as a fluffy white solid. ESIMS found for C₇H₇BF₂N₄O₂ m/z 229.05 (M+H).

The following intermediates were prepared in accordance with the procedure described in the above Scheme 9.

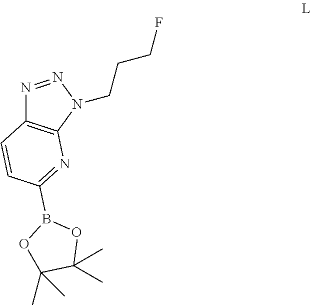 [SMILES: CC1(C)OB(c2ccc3nnn(CCCF)c3n2)OC1(C)C]

3-(3-Fluoropropyl)-5-(4,4,5,5-tetramethyl-1,3,2-dioxaborolan-2-yl)-3H-[1,2,3]triazolo[4,5-b]pyridine (L): Yellow solid (1.79 g, 7.991 mmol). ¹H NMR (400 MHz, CD₃OD) δ ppm 2.34 (2H, br s), 4.31-4.56 (2H, m), 4.85 (4H, br s), 7.86 (1H, d, J=8.50 Hz), 8.37 (1H, br s); ESIMS found for C₁₄H₂₀BFN₄O₂ m/z 307.1 (M+H).

Preparation of intermediate cis-3-amino-1-ethylcyclobutan-1-ol (LIX) is depicted below in Scheme 10.

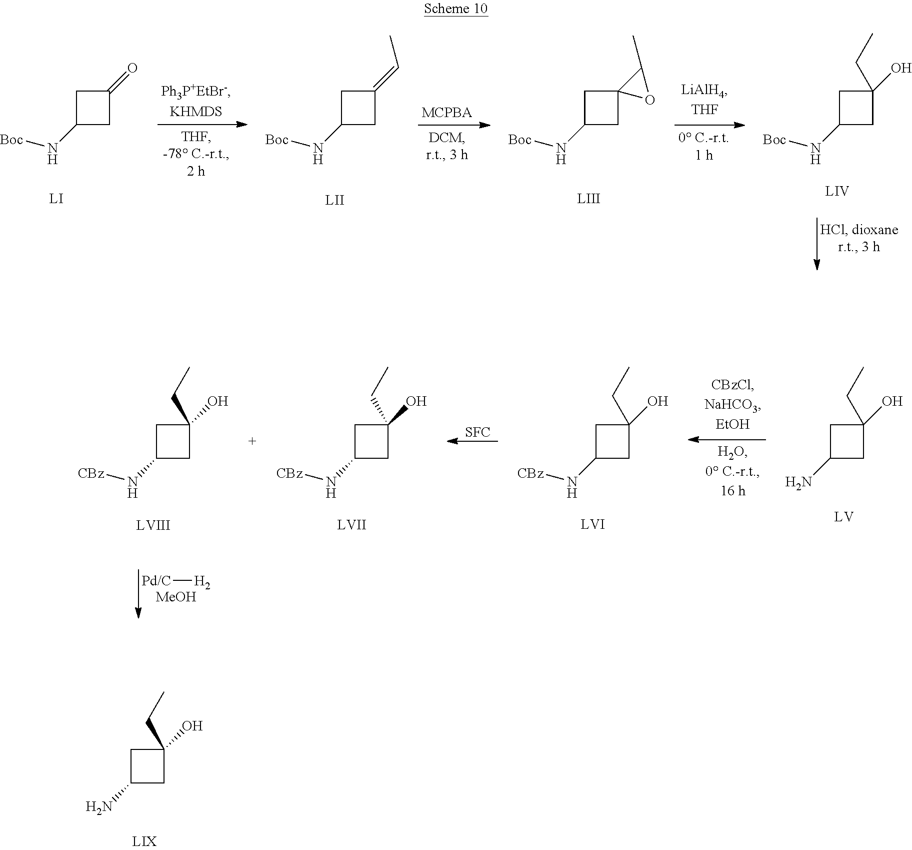

### Step 1

To a cloudy solution of ethyltriphenylphosphonium bromide (30.08 g, 81.08 mmol) in THF (240 mL) at −78° C. under N₂ was added a 1 M solution of KHMDS in THF (80 mL, 81.08 mmol) at −78° C. The mixture was stirred for 30 minutes before adding tert-butyl (3-oxocyclobutyl)carbamate (LI) (10 g, 54.05 mmol) in THF (50 mL) dropwise at −78° C. The reaction mixture was stirred at room temperature for 2 h. The reaction solution was quenched with brine (200 mL) and extracted with EtOAc (2×200 mL). The organic layer was washed with H₂O (150 mL) and brine (150 mL), then separated, dried over anhydrous Na₂SO₄, and concentrated in vacuo. The crude was purified by silica gel column chromatography (silica, 100-200 mesh, 10% EtOAc/PE) to obtain tert-butyl (3-ethylidenecyclobutyl)carbamate (LII) (1.5 g, 7.603 mmol, 14.1% yield) as an off-white solid. ¹H NMR (400 MHz, CDCl₃) δ ppm 1.37 (s, 9H), 1.43 (dd, J=7.0, 3.4 Hz, 3H), 2.49-2.29 (m, 2H), 2.93 (s, 2H), 4.05 (s, 1H), 4.69 (s, 1H), 5.15 (s, 1H).

### Step 2

To a solution of tert-butyl (3-ethylidenecyclobutyl)carbamate (LII) (1.0 g, 5.08 mmol) in DCM (40 mL) at 0° C. was added MCPBA (1.75 g, 10.15 mmol). The mixture was stirred at room temperature for 3 h. The mixture was quenched with brine (50 mL) and extracted with DCM (2×80 mL). The organic layer was separated, then washed with saturated aqueous NaHCO₃ solution (50 mL) and brine (50 mL). The organic layer was separated, dried over anhydrous Na₂SO₄, and concentrated in vacuo to give tert-butyl (2-methyl-1-oxaspiro[2.3]hexan-5-yl)carbamate (LIII) (1.0 g, 4.689 mmol, 92.3% yield) as a white solid which was used in the next step without further purification. ESIMS found for C₁₁H₁₉NO₃ m/z 158.1 (M−^{t}Bu+H).

### Step 3

To a solution of tert-butyl (2-methyl-1-oxaspiro[2.3]hexan-5-yl)carbamate (LIII) (1.0 g, 4.69 mmol) in THF (10 mL) at 0° C. was added LiAlH₄ (6 mL, 6.17 mmol). The mixture was stirred at room temperature for 1 h. The mixture was added sodium sulfate decahydrate and then filtered through a Celite®* pad, and the filtrate was concentrated to give the crude product. The crude product was chromatographed on silica gel (silica, 100-200 mesh, 30% EtOAc/PE) to give the tert-butyl (3-ethyl-3-hydroxycyclobutyl)carbamate (LIV) (200 mg, 0.929 mmol, 19.8% yield) as a white oil. ¹H NMR (400 MHz, DMSO-d₆) δ ppm 0.79 (dt, J=19.6, 7.4 Hz, 3H), 1.36 (s, 9H), 1.43 (dd, J=14.4, 7.2 Hz, 2H), 1.81 (dd, J=14.2, 5.6 Hz, 2H), 2.26-2.02 (m, 2H), 4.05-3.37 (m, 1H), 4.60 (d, J=45.8 Hz, 1H), 6.90 (t, J=73.8 Hz, 1H).

### Step 4

tert-Butyl (3-ethyl-3-hydroxycyclobutyl)carbamate (LIV) (500 mg, 2.33 mmol) was dissolved in a solution of 4 M HCl in dioxane (5 mL). The mixture was stirred at room temperature for 1 h. The mixture was concentrated and dried under vacuum to produce desired 3-amino-i-ethylcyclobutan-1-ol (LV) (500 mg, assuming quantitative) as a yellow oil which was used in the next step without further purification.

### Step 5

To a solution of crude 3-amino-1-ethylcyclobutan-1-ol (LV) (500 mg, 2.33 mmol) in mixture of EtOAc (20 mL) and aqueous NaHCO₃ (10 mL) at 0° C. was added benzyl chloroformate (515.7 mg, 3.02 mmol). The mixture was stirred at room temperature for 16 h. The mixture was quenched with brine (50 mL) and extracted with EtOAc (2×100 mL). The organic layer was separated, dried over anhydrous Na₂SO₄, and concentrated in vacuo. The crude product was chromatographed on silica gel (silica, 100-200 mesh, 30% EtOAc/PE) to yield the racemic benzyl (3-ethyl-3-hydroxycyclobutyl)carbamate (LVI) (450 mg, 1.805 mmol, 77.5% yield) as a white solid. ¹H NMR (400 MHz, DMSO-d₆) δ ppm 0.80 (dt, J=19.6, 7.4 Hz, 3H), 1.44 (dt, J=13.8, 7.0 Hz, 2H), 1.90-1.77 (m, 2H), 2.30-2.06 (m, 2H), 4.20-3.39 (m, 1H), 4.66 (d, J=47.4 Hz, 1H), 4.99 (s, 2H), 7.54-7.09 (m, 6H); ESIMS found for C₁₄H₁₉NO₃ m/z 250.1 (M+H).

Racemic benzyl (3-ethyl-3-hydroxycyclobutyl)carbamate (LVI) (2.5 g, 10.0 mmol) was purified by SFC (Table 2) to afford the benzyl (cis-3-ethyl-3-hydroxycyclobutyl)carbamate (LVIII) (1.8 g, 7.220 mmol) as a white solid ¹H NMR (400 MHz, DMSO-d₆) δ ppm 0.82 (t, J=7.2 Hz, 3H), 1.43 (q, J=7.2 Hz, 2H), 1.85 (td, J=8.8, 2.4 Hz, 2H), 2.33-2.14 (m, 2H), 3.59-3.40 (m, 1H), 4.72 (s, 1H), 4.99 (s, 2H), 7.60-7.09 (m, 6H), ESIMS found for C₁₄H₁₉NO₃ m/z 250.1 (M+H), and benzyl (trans-3-ethyl-3-hydroxycyclobutyl)carbamate (LVII) (0.3 g, 1.203 mmol) as a white solid.

TABLE 2
Chiral pre-SFC Method

| System: | Waters SFC 150 | |
|---|---|---|
| Column name: | CHIRALPAK ® IG (Daicel Corporation) | |
| Column size: | 250*25 mm 10 μm | |
| Mobile Phase A: | Supercritical CO₂, | |
| Mobile Phase B: | MeOH (+0.1% 7.0 mol/L Ammonia in MeOH) | |
| A:B: | 80:20 | |
| Wavelength: | 214 | nm |
| Flow: | 120 | mL/min |
| Column temp: | RT | |
| Back Pressure: | 100 | bar |
| Injection: | 2 | mL |
| Cycle time: | 7.04 | min |

| | |
|---|---|
| Solvent: | MEOH: redistilled grade |
| | Supercritical CO₂: Food grade |
| Preparation of | sample was dissolved in about 95 mL MeOH |
| sample solution: | |

### Step 6

To a solution of benzyl (cis-3-ethyl-3-hydroxycyclobutyl)carbamate (LVIII) (1.8 g, 7.22 mmol) in MeOH (50 mL) was added Pd/C (360 mg). The mixture was stirred at room temperature under H₂ (1 atm) for 16 h. The mixture was filtered through Celite®, and the filtrate was concentrated to give cis-3-amino-1-ethylcyclobutan-1-ol (LIX) (540 mg, 4.688 mol, 64.9% yield) as a yellow solid. ¹H NMR (400 MHz, DMSO-d₆) δ ppm 0.80 (t, J=7.4 Hz, 3H), 1.37 (q, J=7.4 Hz, 2H), 1.64-1.54 (m, 2H), 2.27-2.17 (m, 2H), 2.85-2.73 (m, 1H), 3.17 (s, 1H).

The following intermediate was prepared in accordance with the procedure described in the above Scheme 10.

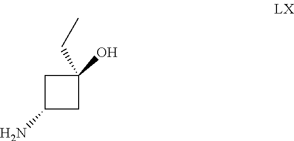

trans-3-Amino-1-ethylcyclobutan-1-ol (LX): ¹H NMR (400 MHz, DMSO-d₆) δ ppm 0.77 (t, J=7.4 Hz, 3H), 1.46 (q, J=7.4 Hz, 2H), 1.58-1.50 (m, 2H), 2.12-2.03 (m, 2H), 3.17 (s, 1H), 3.52-3.38 (m, 1H).

Preparation of intermediate (3R,4S)-3-fluoro-1-(oxetan-3-yl)piperidin-4-amine (LXIV) is depicted below in Scheme 11.

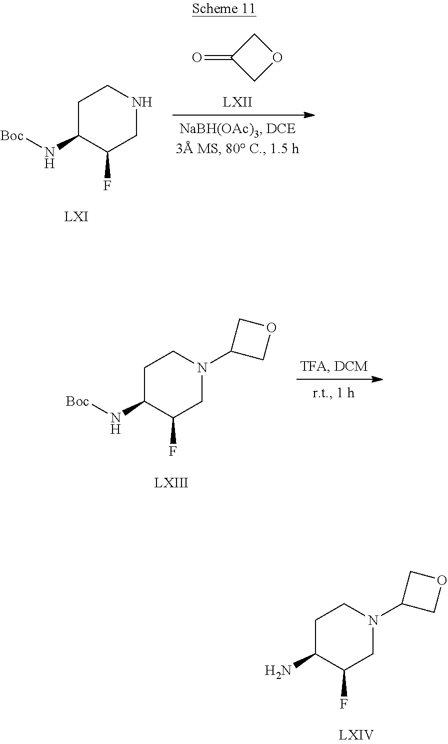 [SMILES: O=C1COC1 ; CC(C)(C)OC(=O)N[C@H]1CCNC[C@H]1F ; CC(C)(C)OC(=O)N[C@H]1CCN(C2COC2)C[C@H]1F ; N[C@H]1CCN(C2COC2)C[C@H]1F]

### Step 1

2-Methyl-2-propanyl[(3R,4S)-3-fluoro-4-piperidinyl]carbamate (LXI) (commercially available from PharmaBlock (USA), Inc.) (1 g, 4.58 mmol) and 3 Å MS (5 beads) were suspended in dry DCE (10 mL) before adding oxetan-3-one (LXII) (805 μL, 13.74 mmol). The reaction was stirred at 80° C. for 1 h. NaBH(OAc)₃ (1.5 g, 7.08 mmol) was added whilst hot and the reaction was stirred for a further 30 min. The reaction mixture was poured into a mixture of saturated aqueous NaHCO₃ solution and DCM, and the organic layer was separated. The aqueous layer was extracted with DCM (×3) and the combined organic layers were reduced in vacuo to give the product tert-butyl N-[(3R,4S)-3-fluoro-1-(oxetan-3-yl)piperidin-4-yl]carbamate (LXIII) (1.259 g, 4.589 mmol, 100.2% yield), assuming quantitative yield, as a viscous orange oil and was used without purification. ESIMS found for C₁₃H₂₃FN₂O₃ m/z 275.15 (M+H).

### Step 2

To a solution of tert-butyl N-[(3R,4S)-3-fluoro-1-(oxetan-3-yl)piperidin-4-yl]carbamate (LXIII) (1.26 g, 4.59 mmol) in DCM (16 mL) was added TFA (4 mL) The reaction was stirred at room temperature for 1 h. The reaction mixture was blown dry, and the product was directly purified by RP chromatography (H₂O with 0.1% formic acid). Appropriate fractions were pooled and lyophilized to give a pale orange viscous oil. TFA was still detected, therefore, the product was dissolved in 7.0 M NH₃ in MeOH and dry loaded onto Celite® and then purified by column chromatography (0→5% 7.0 M NH₃ in MeOH/CHCl₃) to produce (3R,4S)-3-fluoro-1-(oxetan-3-yl)piperidin-4-amine (LXIV) (413 mg, 2.371 mmol, 51.7% yield) as a pale-yellow oil. ESIMS found for C₈H₁₅FN₂O m/z 175.1 (M+H).

### Example 1

Preparation of 5-(3-(2,2-Difluoroethyl)-3H-[1,2,3]triazolo[4,5-b]pyridin-5-yl)-6-fluoro-N²-((3R,4S)-3-fluoro-1-(oxetan-3-yl)piperidin-4-yl)pyrrolo[2,1-f][1,2,4]triazine-2,4-diamine (260) is depicted below in Scheme 12.

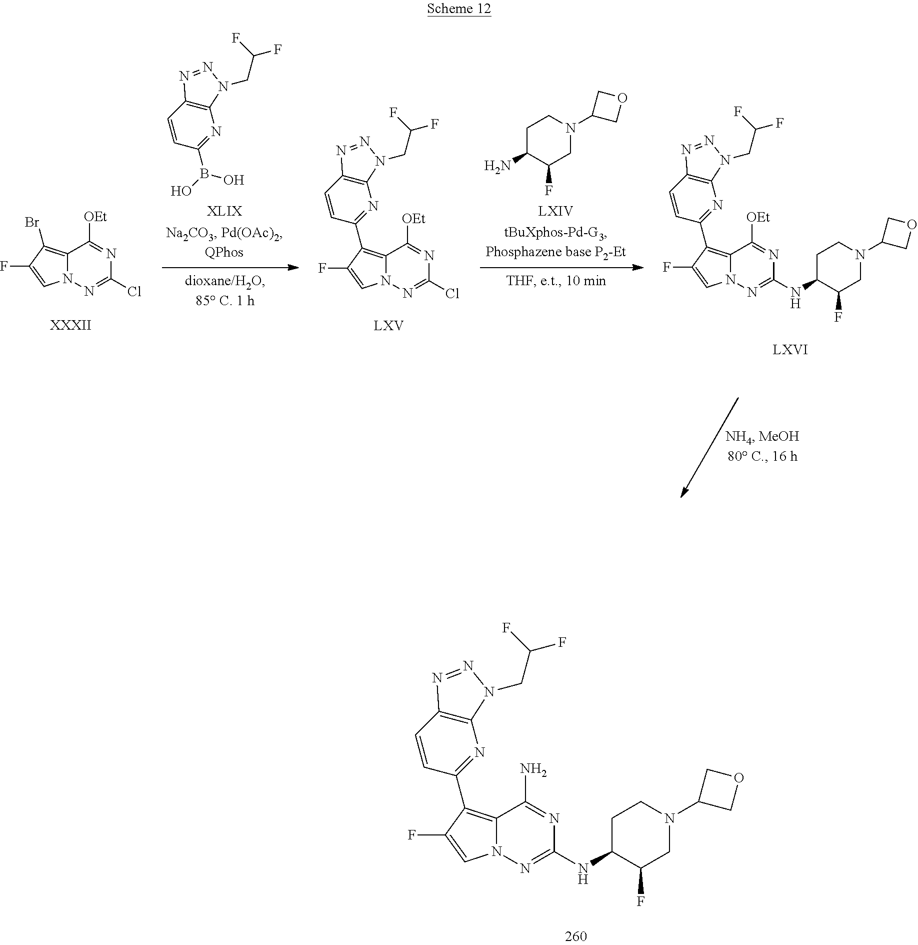

### Step 1

5-Bromo-2-chloro-4-ethoxy-6-fluoropyrrolo[2,1-f][1,2,4]triazine (XXXII) (250 mg, 0.85 mmol), [3-(2,2-difluoroethyl)triazolo[4,5-b]pyridin-5-yl]boronic acid (XLIX) (201 mg, 0.88 mmol), Pd(OAc)₂ (11 mg, 0.05 mmol), QPhos (59 mg, 0.08 mmol) and Na₂CO₃ (270 mg, 2.55 mmol) were suspended in 1,4-dioxane (3 mL) and water (1.5 mL). The reaction mixture was purged with argon for 5 min, and the reaction was heated to 85° C. for 1 h. The reaction mixture was loaded onto Celite® and purified by column chromatography (0→20% EtOAc/hexanes) to yield 5-(2-chloro-4-ethoxy-6-fluoropyrrolo[2,1-f][1,2,4]triazin-5-yl)-3-(2,2-difluoroethyl)triazolo[4,5-b]pyridine (LXV) (133 mg, 0.334 mmol, 39.4% yield) as a pale-red solid. ESIMS found for C₁₅H₁₁ClF₃N₇O m/z 398.1 (M+H).

### Step 2

5-(2-Chloro-4-ethoxy-6-fluoropyrrolo[2,1-f][1,2,4]triazin-5-yl)-3-(2,2-difluoroethyl)triazolo[4,5-b]pyridine (LXV) (133 mg, 0.33 mmol), (3R,4S)-3-fluoro-1-(oxetan-3-yl)piperidin-4-amine (LXIV) (68 mg, 0.39 mmol), and tBuXphos-Pd-G3 (25 mg, 0.03 mmol) were suspended in dry THF (3 mL) and the solution was degassed with Ar for 3 min. Phosphazene base P2-Et (134 μL, 0.4 mmol) was then added slowly down the sides of the vessel and the reaction was stirred at room temperature for 10 min. The crude reaction mixture was purified by column chromatography (0→30% EtOAc/hexanes) to give 5-(3-(2,2-difluoroethyl)-3H-[1,2,3]triazolo[4,5-b]pyridin-5-yl)-4-ethoxy-6-fluoro-N-((3R,4S)-3-fluoro-1-(oxetan-3-yl)piperidin-4-yl)pyrrolo[2,1-f][1,2,4]triazin-2-amine (LXVI) (58 mg, 0.108 mmol, 32.4% yield) as a beige solid. ESIMS found for C₂₃H₂₅F₄N₉O₂ m/z 536.2 (M+H).

### Step 3

5-(3-(2,2-Difluoroethyl)-3H-[1,2,3]triazolo[4,5-b]pyridin-5-yl)-4-ethoxy-6-fluoro-N-((3R,4S)-3-fluoro-1-(oxetan-3-yl)piperidin-4-yl)pyrrolo[2,1-f][1,2,4]triazin-2-amine (LXVI) (58 mg, 0.11 mmol) was suspended in 7 M ammonia in MeOH (3 mL, 21 mmol). The reaction was stirred in a sealed tube for 16 h at 80° C. The reaction mixture was loaded onto Celite® and purified by column chromatography (0→4% 7.0 M NH₃ in MeOH/CHCl₃). The product was further purified using RP column chromatography (0→50% MeCN/H₂O (0.1% formic acid)) to produce 5-(3-(2,2-Difluoroethyl)-3H-[1,2,3]triazolo[4,5-b]pyridin-5-yl)-6-fluoro-N²-((3R,4S)-3-fluoro-1-(oxetan-3-yl)piperidin-4-yl)pyrrolo[2,1-f][1,2,4]triazine-2,4-diamine (260) (10 mg, 0.0120 mmol, 18.2% yield) as a yellow fluffy solid. ¹H NMR (499 MHz, DMSO-d₆) δ ppm 1.70 (1H, br dd, J=8.62, 3.97 Hz), 1.87 (1H, qd, J=12.09, 3.42 Hz), 1.94-2.02 (1H, m), 2.11 (1H, dd, J=37.05, 12.59 Hz), 2.74 (1H, br d, J=12.05 Hz), 2.90-3.03 (1H, m), 3.48 (1H, quin, J=6.43 Hz), 3.71-3.89 (1H, m), 4.39 (1H, t, J=6.16 Hz), 4.45 (1H, t, J=6.16 Hz), 4.53 (2H, td, J=6.50, 2.60 Hz), 4.86 (1H, d, J=49.35 Hz), 5.19 (2H, td, J=16.08, 2.33 Hz), 6.19 (1H, d, J=8.21 Hz), 7.68 (1H, d, J=2.19 Hz), 7.74 (1H, br s), 7.94 (1H, d, J=9.03 Hz), 8.67 (1H, d, J=9.03 Hz), 9.70 (1H, br s); ESIMS found for C₂₁H₂₂F₄N₁₀O m/z 507.2 (M+1).

### Example 2

Preparation of N²-((3R,4S)-3-fluoro-1-(oxetan-3-yl)piperidin-4-yl)-5-(3-(2-fluoroethyl)-3H-[1,2,3]triazolo[4,5-b]pyridin-5-yl)-N⁴-methylpyrrolo[2,1-f][1,2,4]triazine-2,4-diamine (236) is depicted below in Scheme 13.

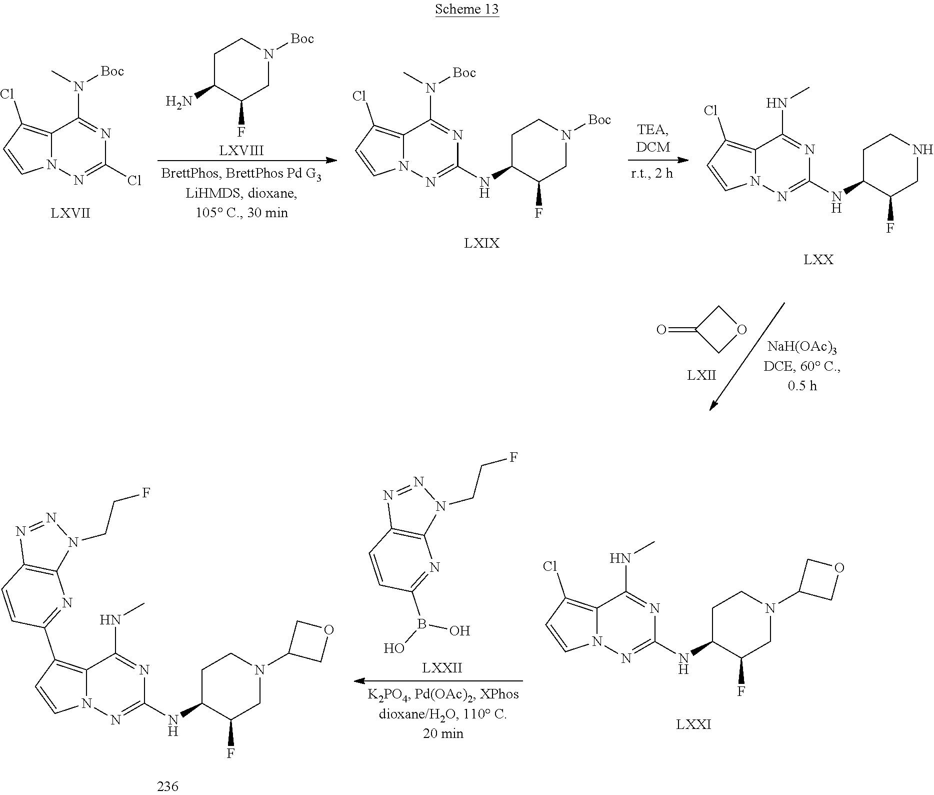

### Step 1

To an oven-dried microwave vial containing tert-Butyl N-(2,5-dichloropyrrolo[2,1-f][1,2,4]triazin-4-yl)-N-methylcarbamate (LXVII) (400 mg, 1.26 mmol), tert-butyl (3R,4S)-4-amino-3-fluoropiperidine-1-carboxylate (LXVIII) (358 mg, 1.64 mmol), BrettPhos Pd G3 (92 mg, 0.1 mmol), BrettPhos (54 mg, 0.1 mmol) was added dry 1,4-dioxane (7 mL). A 1 M solution of LiHMDS in THF (3.16 mL, 3.16 mmol) was added, and the suspension was purged and sonicated with Ar for 5 min. The reaction was then heated to 105° C. for 30 min. The reaction mixture was added to Celite®* and purified by column chromatography (0→20% EtOAc/hexanes) to yield tert-butyl (3R,4S)-4-((4-((tert-butoxycarbonyl)(methyl)amino)-5-chloropyrrolo[2,1-f][1,2,4]triazin-2-yl)amino)-3-fluoropiperidine-1-carboxylate (LXIX) (374 mg, 0.750 mmol, 59.4% yield) as a foamy beige solid. ESIMS found for C₁₇H₂₄ClFN₆O₂ m/z 399.2 (M−Boc+H).

### Step 2

To a solution of tert-butyl (3R,4S)-4-((4-((tert-butoxycarbonyl)(methyl) amino)-5-chloropyrrolo[2,1-f][1,2,4]triazin-2-yl)amino)-3-fluoropiperidine-1-carboxylate (LXIX) (374 mg, 0.75 mmol) in DCM (8 mL) was added TFA (2 mL). The reaction was stirred at room temperature for 2 h. The mixture was evaporated, and the crude product was purified by column chromatography (0→5% 7.0 M NH₃ in MeOH/CHCl₃) to give 5-chloro-N²-((3R,4S)-3-fluoropiperidin-4-yl)-N⁴-methylpyrrolo[2,1-f][1,2,4]triazine-2,4-diamine (LXX) (226 mg, 0.757 mmol, 100.9% yield) as a fluffy white solid. ESIMS found for C₁₂H₁₆C₁FN₆ m/z 299.05 (M+1).

### Step 3

5-chloro-N²-((3R,4S)-3-fluoropiperidin-4-yl)-N⁴-methylpyrrolo[2,1-f][1,2,4]triazine-2,4-diamine (LXX) (80 mg, 0.27 mmol) was suspended in dry DCE (2 mL) before adding oxetan-3-one (LXII) (50 μL, 0.85 mmol). NaH(OAc)₃ (85 mg, 0.4 mmol) was added and the reaction was heated to 60° C. for 30 min. LCMS showed SM remaining, therefore, an additional portion of oxetan-3-one (LXII) (50 μL, 0.85 mmol) and NaH(OAc)₃ (85 mg, 0.4 mmol) were added and the reaction was heated 60° C. for another 5 min. LCMS showed the reaction to be complete. The reaction mixture was loaded onto Celite® and purified by column chromatography (0→2% MeOH/CHCl₃) to give 5-chloro-N²-((3R,4S)-3-fluoro-1-(oxetan-3-yl)piperidin-4-yl)-N⁴-methylpyrrolo[2,1-f][1,2,4]triazine-2,4-diamine (LXXI) (71 mg, 0.200 mmol, 74.7% yield) as a pale-yellow solid. ESIMS found for C₁₅H₂₀ClFN₆O m/z 355.2 (M+1).

### Step 4

To a solution of 5-chloro-N²-((3R,4S)-3-fluoro-1-(oxetan-3-yl)piperidin-4-yl)-N⁴-methylpyrrolo[2,1-f][1,2,4]triazine-2,4-diamine (LXXI) (13 mg, 0.04 mmol) in 1,4-dioxane (300 μL) was added a 2 M aqueous solution of K₂PO₄ (55 μL, 0.11 mmol). The reaction was purged and sonicated with Ar for 5 min. The reaction was then heated to 110° C. and then removed from the heat before adding [3-(2-fluoroethyl)triazolo[4,5-b]pyridin-5-yl]boronic acid (LXXII) (15 mg, 0.07 mmol), Pd(OAc)₂ (1 mg, 0. mmol), and XPhos (3 mg, 0.01 mmol). The reaction was sealed and stirred at 110° C. for 20 min. The mixture was loaded onto Celite® and purified by column chromatography (0→4% MeOH/CHCl₃). The product was further purified by HPLC (0→30% MeCN/H₂O (0.1% formic acid)) to produce N²-((3R,4S)-3-Fluoro-1-(oxetan-3-yl)piperidin-4-yl)-5-(3-(2-fluoroethyl)-3H-[1,2,3]triazolo[4,5-b]pyridin-5-yl)-N⁴-methylpyrrolo[2,1-f][1,2,4]triazine-2,4-diamine (236) (14 mg, 0.029 mmol, 78.9% yield) as a fluffy yellow solid. ¹H NMR (499 MHz, DMSO-d₆) δ ppm 1.67-1.75 (1H, m), 1.88 (1H, qd, J=12.05, 3.83 Hz), 1.96-2.04 (1H, m), 2.14 (1H, dd, J=37.00, 12.87 Hz), 2.75 (1H, br d, J=10.95 Hz), 2.92-3.03 (1H, m), 3.13 (3H, d, J=4.65 Hz), 3.49 (1H, quin, J=6.37 Hz), 3.74-3.91 (1H, m), 4.40 (1H, t, J=6.16 Hz), 4.46 (1H, t, J=6.16 Hz), 4.54 (2H, td, J=6.57, 3.29 Hz), 4.91 (1H, d, J=50.15 Hz), 4.97-5.05 (2H, m), 5.08 (2H, s), 6.15 (1H, d, J=7.94 Hz), 7.32 (1H, d, J=3.01 Hz), 7.43 (1H, d, J=3.01 Hz), 8.08 (1H, d, J=9.03 Hz), 8.53 (1H, d, J=9.04 Hz), 11.00 (1H, q, J=4.47 Hz); ESIMS found for C₂₂H₂₆F₂N₁₀O m/z 243.2 (M/2+1).

### Example 3

Preparation of 1-((3R,4S)-3-Fluoro-4-((5-(3-(2-fluoroethyl)-3H-[1,2,3]triazolo[4,5-b]pyridin-5-yl)-4-(methylamino)pyrrolo[2,1-f][1,2,4]triazin-2-yl)amino)piperidin-1-yl)ethan-1-one (235) is depicted below in Scheme 14.

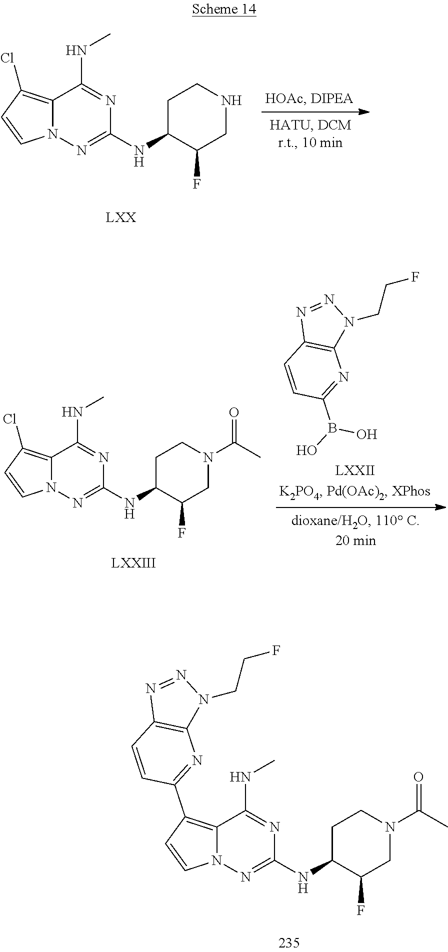 [SMILES: CNc1nc(N[C@H]2CCNC[C@H]2F)nn2ccc(Cl)c12 ; CNc1nc(N[C@H]2CCN(C(C)=O)C[C@H]2F)nn2ccc(Cl)c12 ; OB(O)c1ccc2nnn(CCF)c2n1 ; CNc1nc(N[C@H]2CCN(C(C)=O)C[C@H]2F)nn2ccc(-c3ccc4nnn(CCF)c4n3)c12]

### Step 1

To a solution of HATU (107 mg, 0.28 mmol) in DCM (2 mL) was added HOAc (19 μL, 0.33 mmol) and DIPEA (98 μL, 0.56 mmol) were added and the reaction was stirred for 5 min. 5-Chloro-N²-((3R,4S)-3-fluoropiperidin-4-yl)-N⁴-methylpyrrolo[2,1-f][1,2,4]triazine-2,4-diamine (LXX) (70 mg, 0.23 mmol) was then added and the reaction was stirred at room temperature for 10 min. The reaction mixture was purified by column chromatography (0→2% MeOH/CHCl₃) to produce 1-((3R,4S)-4-((5-chloro-4-(methylamino)pyrrolo[2,1-f][1,2,4]triazin-2-yl)amino)-3-fluoropiperidin-1-yl)ethan-1-one (LXXIII) (85 mg, 0.249 mmol, 106.5% yield) as a fluffy white solid. ESIMS found for C₁₄H₁₈ClFN₆O m/z 341.1 (M+H).

### Step 2

To a solution of 1-((3R,4S)-4-((5-chloro-4-(methylamino)pyrrolo[2,1-f][1,2,4]triazin-2-yl)amino)-3-fluoropiperidin-1-yl)ethan-1-one (LXXIII) (13 mg, 0.04 mmol) in 1,4-dioxane (300 μL) was added a 2 M aqueous solution of K₂PO₄ (57 μL, 0.11 mmol) and the reaction was purged and sonicated with Ar for 5 min. The reaction was the heated to 110° C. and then removed from the heat before adding [3-(2-fluoroethyl)triazolo[4,5-b]pyridin-5-yl]boronic acid (LXXII) (16 mg, 0.08 mmol), Pd(OAc)₂ (1 mg, 0.0045 mmol), and XPhos (3 mg, 0.01 mmol). The reaction was sealed and stirred at 110° C. for 20 min. The reaction mixture was loaded onto Celite® and the product was purified by column chromatography (0→3% MeOH/CHCl₃). The product was further purified by HPLC (0→60% MeCN/H₂O (0.1% formic acid)) to give 1-((3R,4S)-3-Fluoro-4-((5-(3-(2-fluoroethyl)-3H-[1,2,3]triazolo[4,5-b]pyridin-5-yl)-4-(methylamino)pyrrolo[2,1-f][1,2,4]triazin-2-yl)amino)piperidin-1-yl)ethan-1-one (235) (9 mg, 0.019 mmol, 50.1% yield) as a fluffy yellow solid after lyophilization. ¹H NMR (499 MHz, DMSO-d₆) δ ppm 1.59-1.89 (2H, m), 1.96-2.07 (3H, m), 2.59-2.78 (1H, m), 2.79-2.95 (1H, m), 3.13 (3H, d, J=4.65 Hz), 3.83-4.00 (1H, m), 4.01-4.19 (1H, m), 4.40-4.75 (1H, m), 4.89-5.02 (1H, m), 4.97-5.06 (2H, m), 5.08 (2H, s), 6.27 (1H, d, J=7.94 Hz), 7.33 (1H, d, J=3.29 Hz), 7.44 (1H, d, J=3.01 Hz), 8.09 (1H, d, J=9.31 Hz), 8.54 (1H, d, J=9.03 Hz), 11.01 (1H, q, J=4.95 Hz); ESIMS found for C₂₁H₂₄F₂N₁₀O m/z 471.2 (M+1).

### Example 4

Preparation of 5-(3-(2,2-difluoroethyl)-3H-imidazo[4,5-b]pyridin-5-yl)-N²-((3R,4S)-3-fluoro-1-(2-methoxyethyl)piperidin-4-yl)pyrrolo[2,1-f][1,2,4]triazine-2,4-diamine (121) is depicted below in Scheme 15.

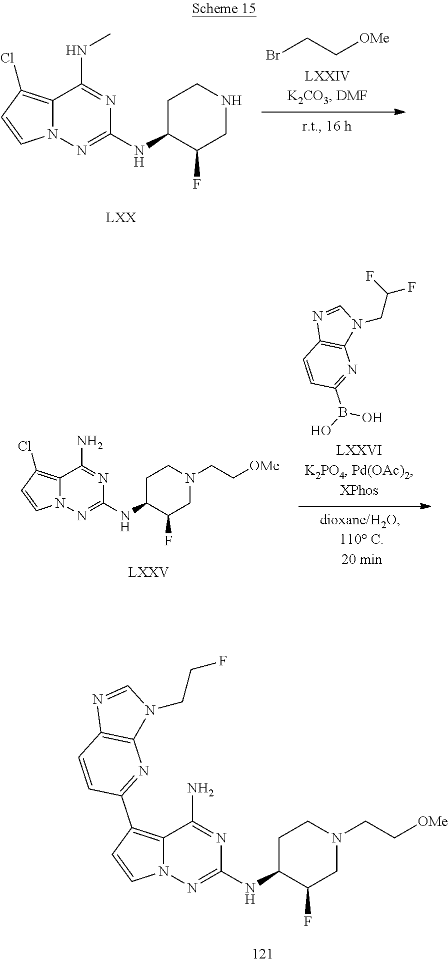 [SMILES: CNc1nc(N[C@H]2CCNC[C@H]2F)nn2ccc(Cl)c12 ; COCCBr ; OB(O)c1ccc2ncn(CC(F)F)c2n1 ; COCCN1CC[C@H](Nc2nc(N)c3c(Cl)ccn3n2)[C@H](F)C1 ; COCCN1CC[C@H](Nc2nc(N)c3c(-c4ccc5ncn(CCF)c5n4)ccn3n2)[C@H](F)C1]

### Step 1

To a stirring solution of 2-bromoethyl methyl ether (LXXIV) (30 μL, 0.32 mmol) in DMF (1 mL) was added K₂CO₃ (51 mg, 0.37 mmol) and 5-chloro-N²-((3R,4S)-3-fluoropiperidin-4-yl)-N⁴-methylpyrrolo[2,1-f][1,2,4]triazine-2,4-diamine (LXX) (95 mg, 0.33 mmol). The reaction mixture was stirred at room temperature for 16 h before pouring mixture into a mixture of EtOAc and water. The aqueous layer was extracted with EtOAc (2×), the combined organic layers were washed with brine, dried over MgSO₄, filtered and concentrated. The crude product was purified via column chromatography (8 g of silica gel) (0→8% 7 N NH₃ in MeOH/CHCl₃) to yield 5-chloro-N²-((3R,4S)-3-fluoro-1-(2-methoxyethyl)piperidin-4-yl)pyrrolo[2,1-f][1,2,4]triazine-2,4-diamine (LXXV) (91 mg, 0.266 mmol, 79.6% yield) as an amber viscous solid. ESIMS found for C₁₄H₂₀ClFN₆O m/z 343.1 (M+1).

### Step 2

To a solution of 5-chloro-N²-((3R,4S)-3-fluoro-1-(2-methoxyethyl)piperidin-4-yl)pyrrolo[2,1-f][1,2,4]triazine-2,4-diamine (LXXV) (15 mg, 0.04 mmol) in 1,4-dioxane (0.5 mL) was added a 2 M aqueous solution of K₂PO₄ (66 μL, 0.13 mmol) and the mixture was heated to 110° C. and then removed from the heat before adding [3-(2,2-difluoroethyl)imidazo[4,5-b]pyridin-5-yl]boronic acid (LXXVI) (28.3 mg, 0.12 mmol), Pd(OAc)₂ (2.4 mg, 0.01 mmol), and XPhos (4.8 mg, 0.01 mmol). The reaction was sealed and heated at 110° C. for 30 min. The reaction mixture was loaded onto Celite® and the product was purified via reverse phase chromatography using a 15.5 g ISCO column (0→100% MeCN/H₂O (0.1% formic acid)) to produce 5-(3-(2,2-difluoroethyl)-3H-imidazo[4,5-b]pyridin-5-yl)-N²-((3R,4S)-3-fluoro-1-(2-methoxyethyl) piperidin-4-yl)pyrrolo[2,1-f][1,2,4]triazine-2,4-diamine (121) (22 mg, 0.045 mmol, 51.4% yield) as a white solid after lyophilization. ¹H NMR (499 MHz, DMSO-d₆) δ ppm 1.59-1.71 (1H, m), 1.83 (1H, qd, J=12.18, 3.70 Hz), 2.15 (1H, br t, J=11.09 Hz), 2.29 (1H, dd, J=38.10, 12.59 Hz), 2.51 (2H, t, J=5.75 Hz), 2.82-2.94 (1H, m), 3.08-3.18 (1H, m), 3.24 (3H, s), 3.43 (2H, t, J=5.75 Hz), 3.68-3.85 (1H, m), 4.74 (2H, td, J=16.63, 2.05 Hz), 4.84 (1H, d, J=50.20 Hz), 5.79 (1H, d, J=8.21 Hz), 6.48 (1H, tt, J=54.30, 2.46 Hz), 7.14 (1H, d, J=3.01 Hz), 7.40 (1H, d, J=2.74 Hz), 7.59 (1H, br s), 7.88 (1H, d, J=8.76 Hz), 8.15 (1H, d, J=8.49 Hz), 8.40 (1H, s), 10.57 (1H, br s); ESIMS found for C₂₂H₂₆F₃N₉O m/z 490.2 (M+1).

### Example 5

Preparation of 1-((3R,4S)-4-((4-amino-5-(3-(2,2-difluoroethyl)-3H-[1,2,3]triazolo[4,5-b]pyridin-5-yl)-6-fluoropyrrolo[2,1-f][1,2,4]triazin-2-yl)amino)-3-fluoropiperidin-1-yl)ethan-1-one (258) is depicted below in Scheme 16.

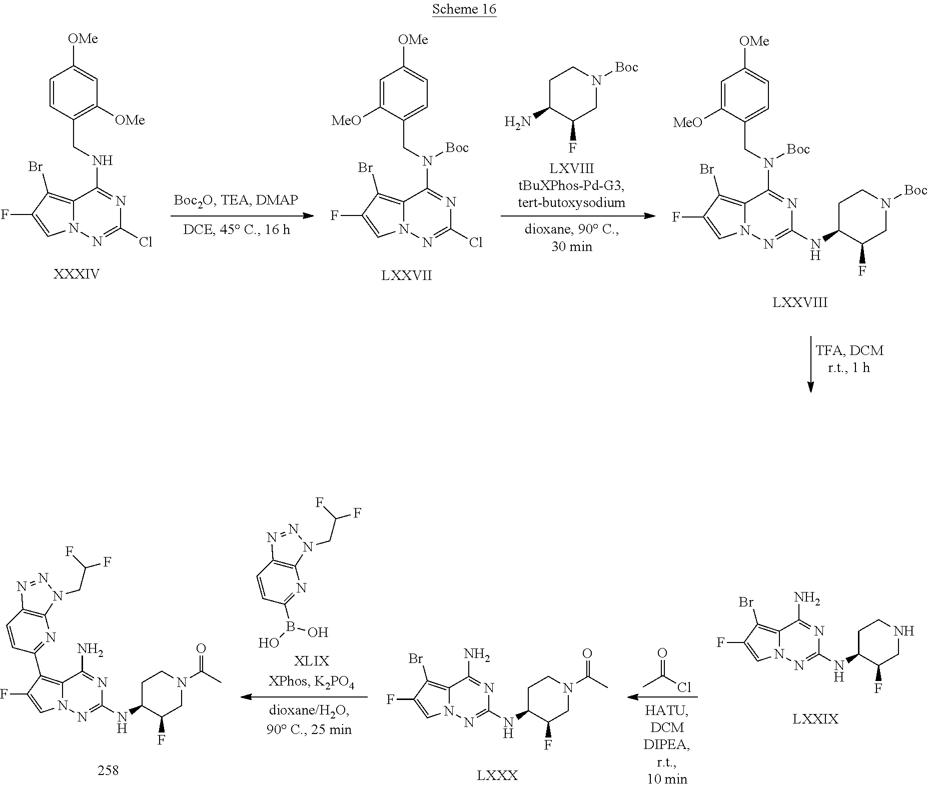

### Step 1

5-Bromo-2-chloro-N-(2,4-dimethoxybenzyl)-6-fluoropyrrolo[2,1-f][1,2,4]triazin-4-amine (XXXIV) (507 mg, 1.22 mmol) and DMAP (75 mg, 0.61 mmol) were dissolved in DCE (5 mL) and di-tert-butyl decarbonate (420 μL, 1.83 mmol) and TEA (520 μL, 3.73 mmol) were added and the reaction was stirred at 45° C. for 16 h. LCMS showed the reaction to be complete. The reaction mixture was loaded onto Celite® and was purified by column chromatography (0→5% EtOAc/hexanes) to yield tert-butyl (5-bromo-2-chloro-6-fluoropyrrolo[2,1-f][1,2,4]triazin-4-yl)(2,4-dimethoxybenzyl)carbamate (LXXVII) (385 mg, 0.747 mmol, 61.2% yield) as a pale-yellow foamy solid. ESIMS found for C₂₀H₂₁BrClFN₄O₄ m/z 415. (M−Boc+1).

### Step 2

tert-Butyl (5-bromo-2-chloro-6-fluoropyrrolo[2,1-f][1,2,4]triazin-4-yl)(2,4-dimethoxybenzyl)carbamate (LXXVII) (100 mg, 0.19 mmol) and ^{t}BuXPhos-Pd-G3 (17 mg, 0.02 mmol) were suspended in dry 1,4-dioxane (2 mL) and the reaction mixture was bubbled with Ar for 2 min. tert-Butyl (3R,4S)-4-amino-3-fluoropiperidine-1-carboxylate (LXVIII) (53 mg, 0.24 mmol) and sodium tert-butoxide (70 mg, 0.73 mmol) were added, the reaction vial was sealed and stirred at 90° C. for 30 min. LCMS showed the reaction to be complete. The reaction mixture poured into a saturated aqueous NH₄Cl solution and DCM, and the organic layer was separated. The aqueous layer was extracted with DCM (×2) and the combined organic layers were dried (MgSO₄) and reduced in vacuo to give the crude product as an orange semi-solid. The product was purified by column chromatography (wet load in DCM, 0→45% EtOAc/hexanes) to give tert-butyl (3R,4S)-4-((5-bromo-4-((tert-butoxycarbonyl)(2,4-dimethoxybenzyl)amino)-6-fluoropyrrolo[2,1-f][1,2,4]triazin-2-yl)amino)-3-fluoropiperidine-1-carboxylate (LXXVIII) (68 mg, 0.098 mmol, 50.3% yield) as a pale-yellow semi-solid. ESIMS found for C₃₀H₃₉BrF₂N₆O₆ did not ionize.

### Step 3

tert-Butyl (3R,4S)-4-((5-bromo-4-((tert-butoxycarbonyl)(2,4-dimethoxybenzyl)amino)-6-fluoropyrrolo[2,1-f][1,2,4]triazin-2-yl)amino)-3-fluoropiperidine-1-carboxylate (LXXVIII) (68 mg, 0.1 mmol) was dissolved in DCM (4 mL) before adding TFA (1 mL). The reaction was stirred at room temperature for 1 h. LCMS showed the reaction to be complete. The reaction mixture was blown dry and the resulting green semi-solid was purified by column chromatography (0→3% hold 7.0 M NH₃ in MeOH/CHCl₃) to produce 5-bromo-6-fluoro-N²-((3R,4S)-3-fluoropiperidin-4-yl)pyrrolo[2,1-f][1,2,4]triazine-2,4-diamine (LXXIX) (30 mg, 0.086 mmol, 88.6% yield) as a pale-yellow semi-solid. ESIMS found for C₁₁H₁₃BrF₂N₆ m/z 347.0 (M+1).

### Step 4

HATU (39 mg, 0.1 mmol) was dissolved in DCM (300 μL) and acetic acid (13 μL, 0.23 mmol) and DIPEA (37 μL, 0.21 mmol) were added. The reaction was stirred at room temperature for 5 min. 5-Bromo-6-fluoro-N²-((3R,4S)-3-fluoropiperidin-4-yl)pyrrolo[2,1-f][1,2,4]triazine-2,4-diamine (LXXIX) (30 mg, 0.09 mmol) was dissolved in DCM (300 μL) and this was added to the reaction which was stirred at room temperature for 10 min. LCMS confirmed the reaction to be complete. The reaction mixture was added to Celite® and the product was purified by column chromatography (0→3% hold MeOH/CHCl₃) to give 1-((3R,4S)-4-((4-amino-5-bromo-6-fluoropyrrolo[2,1-f][1,2,4]triazin-2-yl)amino)-3-fluoropiperidin-1-yl)ethan-1-one (LXXX) (15 mg, 0.039 mmol, 44.6% yield) as a pale-yellow semi-solid. ESIMS found for C₁₃H₁₅BrF₂N₆O m/z 389.05 (M+1).

### Step 5

1-((3R,4S)-4-((4-amino-5-bromo-6-fluoropyrrolo[2,1-f][1,2,4]triazin-2-yl) amino)-3-fluoropiperidin-1-yl)ethan-1-one (LXXX) (5 mg, 0.01 mmol) and XPhos Pd G3 (0.5 mg, 0.0006 mmol) were suspended in dry 1,4-dioxane (200 μL) before adding a 2 M aqueous solution of K₂PO₄ (19 μL, 0.04 mmol). The reaction was sonicated and bubbled with Ar for 2 min and then heated to 90° C. (3-(2,2-Difluoroethyl)-3H-[1,2,3]triazolo[4,5-b]pyridin-5-yl)boronic acid (XLIX) (6 mg, 0.03 mmol) was added and the reaction was sealed and stirred at 90° C. for 15 min. LCMS showed starting material remaining, therefore, the reaction was removed from the heat and an additional portion of XPhos Pd G3 (0.5 mg, 0.0006 mmol) and a 2 M aqueous solution of K₂PO₄ (19 μL, 0.04 mmol) were added and the reaction was sonicated and bubbled with Ar for 2 min before reheating to 90° C. An additional portion of (3-(2,2-difluoroethyl)-3H-[1,2,3]triazolo[4,5-b]pyridin-5-yl)boronic acid (XLIX) (6 mg, 0.03 mmol) was added and the reaction vial was resealed and stirred at 90° C. for 10 min. LCMS showed the reaction to be complete. The reaction mixture was added to Celite® and the product was purified by column chromatography (0→3% hold MeOH/CHCl₃). The product was further purified using HPLC (0→60% hold MeCN/H₂O in 0.1% formic acid) to yield 1-((3R,4S)-4-((4-amino-5-(3-(2,2-difluoroethyl)-3H-[1,2,3]triazolo[4,5-b]pyridin-5-yl)-6-fluoropyrrolo[2,1-f][1,2,4]triazin-2-yl)amino)-3-fluoropiperidin-1-yl)ethan-1-one (258) (6 mg, 0.012 mmol, 94.8% yield) as a fluffy yellow solid. ¹H NMR (499 MHz, DMSO-d₆) δ ppm 1.59-1.69 (1H, m), 1.71-1.87 (1H, m), 1.99 (3H, s), 2.59-2.73 (1H, m), 3.09-3.24 (1H, m), 3.82-3.96 (1H, m), 3.98-4.17 (1H, m), 4.41-4.72 (1H, m), 4.93 (1H, d, J=49.90 Hz), 5.19 (2H, td, J=16.22, 2.33 Hz), 6.31 (1H, d, J=8.21 Hz), 6.60 (2H, tt, J=54.00, 2.60 Hz), 7.68 (1H, d, J=1.92 Hz), 7.75 (1H, br s), 7.94 (1H, d, J=8.76 Hz), 8.67 (1H, d, J=9.03 Hz), 9.71 (1H, br s); ESIMS found for C₂₀H₂₀F₄N₁₀O m/z 493.15 (M+1).

The following compounds were prepared in accordance with the procedures described in the above Schemes 1-16.

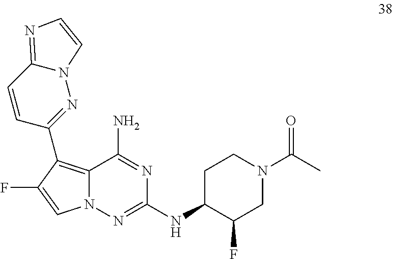 [SMILES: CC(=O)N1CC[C@H](Nc2nc(N)c3c(-c4ccc5nccn5n4)c(F)cn3n2)[C@H](F)C1]

1-((3R,4S)-4-((4-Amino-6-fluoro-5-(imidazo[1,2-b]pyridazin-6-yl)pyrrolo [2,1-f][1,2,4]triazin-2-yl)amino)-3-fluoropiperidin-1-yl)ethan-1-one 38.

Pale-yellow fluffy solid (10 mg, 0.023 mmol, 91.1% yield). ¹H NMR (499 MHz, DMSO-d₆) δ ppm 1.60-1.70 (1H, m), 1.71-1.86 (1H, m), 1.99 (3H, s), 2.61-2.89 (1H, m), 3.13-3.24 (1H, m), 3.82-3.96 (1H, m), 3.97-4.17 (1H, m), 4.40-4.73 (1H, m), 4.92 (1H, d, J=49.05 Hz), 6.27 (1H, d, J=8.21 Hz), 6.662 (1H, s), 7.57 (1H, d, J=8.76 Hz), 7.68 (1H, d, J=2.19 Hz), 7.80 (1H, d, J=1.10 Hz), 7.99 (1H, br s), 8.21 (1H, d, J=9.86 Hz), 8.31 (1H, s); ESIMS found for C₁₉H₁₉F₂N₉O m/z 428.15 (M+1).

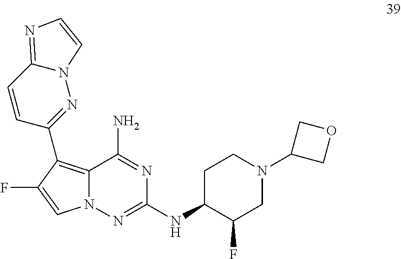 [SMILES: Nc1nc(N[C@H]2CCN(C3COC3)C[C@H]2F)nn2cc(F)c(-c3ccc4nccn4n3)c12]

6-Fluoro-N₂-((3R,4S)-3-fluoro-1-(oxetan-3-yl)piperidin-4-yl)-5-(imidazo[1,2-b]pyridazin-6-yl)pyrrolo[2,1-f][1,2,4]triazine-2,4-diamine 39.

Fluffy pale-yellow solid (4 mg, 0.009 mmol, 91.3% yield). ¹H NMR (499 MHz, DMSO-d₆) δ ppm 1.64-1.74 (1H, m), 1.86 (1H, qd, J=11.96, 3.56 Hz), 1.94-2.03 (1H, m), 2.11 (1H, dd, J=36.75, 12.59 Hz), 2.73 (1H, br d, J=11.77 Hz), 2.97 (1H, br t, J=10.95 Hz), 3.48 (1H, quin, J=6.50 Hz), 3.71-3.87 (1H, m), 4.39 (1H, t, J=6.16 Hz), 4.45 (1H, t, J=6.16 Hz), 4.53 (2H, td, J=6.50, 2.60 Hz), 4.86 (1H, d, J=49.90 Hz), 6.16 (1H, d, J=8.21 Hz), 6.71 (1H, br s), 7.57 (1H, d, J=8.76 Hz), 7.68 (1H, d, J=2.19 Hz), 7.80 (1H, d, J=1.37 Hz), 7.99 (1H, br s), 8.21 (1H, d, J=9.86 Hz), 8.30 (1H, s); ESIMS found for C₂₀H₂₁F₂N₉O m/z 442.15 (M+1).

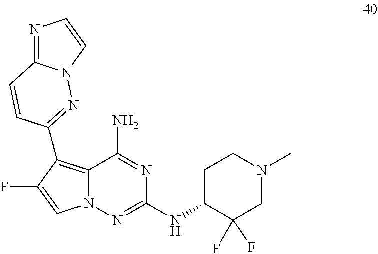 [SMILES: CN1CC[C@@H](Nc2nc(N)c3c(-c4ccc5nccn5n4)c(F)cn3n2)C(F)(F)C1]

(R)—N2-(3,3-Difluoro-1-methylpiperidin-4-yl)-6-fluoro-5-(imidazo[1,2-b]pyridazin-6-yl)pyrrolo[2,1-f][1,2,4]triazine-2,4-diamine 40.

Pale-yellow fluffy solid (3 mg, 0.007 mmol, 32.1% yield). ¹H NMR (499 MHz, DMSO-d₆) δ ppm 1.69-1.87 (2H, m), 2.06-2.18 (1H, m), 2.24 (3H, s), 2.32 (1H, dd, J=26.60, 12.32 Hz), 2.77 (1H, br d, J=11.50 Hz), 2.99-3.10 (1H, m), 4.12-4.29 (1H, m), 6.35 (1H, br d, J=9.58 Hz), 6.79 (1H, br s), 7.57 (1H, d, J=9.58 Hz), 7.70 (1H, d, J=2.19 Hz), 7.80 (1H, s), 8.00 (1H, br s), 8.21 (1H, d, J=9.58 Hz), 8.30 (1H, s); ESIMS found for C₈H₁₈F₃N₉ m/z 418.2 (M+1).

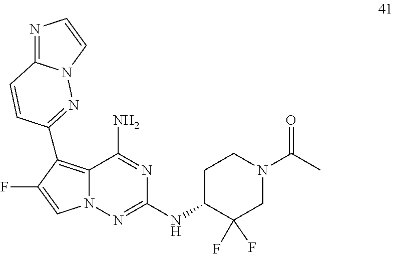 [SMILES: CC(=O)N1CC[C@@H](Nc2nc(N)c3c(-c4ccc5nccn5n4)c(F)cn3n2)C(F)(F)C1]

(R)-1-(4-((4-Amino-6-fluoro-5-(imidazo[1,2-b]pyridazin-6-yl)pyrrolo[2,1-f][1,2,4]triazin-2-yl)amino)-3,3-difluoropiperidin-1-yl)ethan-1-one 41.

Pale-yellow fluffy solid (4 mg, 0.009 mmol, 66.5% yield). ¹H NMR (499 MHz, DMSO-d₆) δ ppm 1.55-1.80 (1H, m), 1.81-1.96 (1H, m), 2.06 (3H, s), 2.97 (1H, br t, J=10.95 Hz), 3.59-3.88 (1H, m), 4.07-4.25 (1H, m), 4.40-4.56 (2H, m), 6.55 (1H, br d, J=9.31 Hz), 6.70 (1H, s), 7.57 (1H, d, J=9.03 Hz), 7.69 (1H, d, J=2.19 Hz), 7.81 (1H, d, J=1.10 Hz), 8.01 (1H, br s), 8.21 (1H, d, J=9.58 Hz), 8.31 (1H, s); ESIMS found for C₁₉H₁₈F₃N₉O m/z 446.15 (M+1).

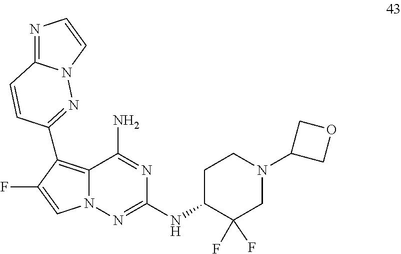 [SMILES: Nc1nc(N[C@@H]2CCN(C3COC3)CC2(F)F)nn2cc(F)c(-c3ccc4nccn4n3)c12]

(R)—N2-(3,3-Difluoro-1-(oxetan-3-yl)piperidin-4-yl)-6-fluoro-5-(imidazo [1,2-b]pyridazin-6-yl)pyrrolo[2,1-f][1,2,4]triazine-2,4-diamine 43.

Pale-yellow fluffy solid (5 mg, 0.011 mmol, 76.4% yield). ¹H NMR (499 MHz, DMSO-d₆) δ ppm 1.70-1.82 (1H, m), 1.82-1.90 (1H, m), 2.11 (1H, br t, J=10.54 Hz), 2.34 (1H, dd, J=26.60, 11.50 Hz), 2.74 (1H, br d, J=11.50 Hz), 2.95-3.06 (1H, m), 3.59 (1H, quin, J=6.37 Hz), 4.19-4.34 (1H, m), 4.43 (2H, dt, J=14.10, 6.09 Hz), 4.54 (2H, td, J=6.57, 3.56 Hz), 6.42 (1H, br d, J=9.58 Hz), 6.71 (1H, br s), 7.57 (1H, d, J=9.03 Hz), 7.69 (1H, d, J=2.19 Hz), 7.80 (1H, d, J=1.10 Hz), 8.00 (1H, br s), 8.21 (1H, d, J=9.58 Hz), 8.30 (1H, s); ESIMS found for C₂₀H₂₀F₃N₉O m/z 460.2 (M+1).

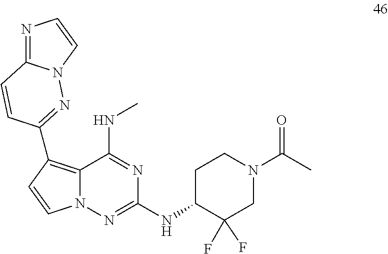 [SMILES: CNc1nc(N[C@@H]2CCN(C(C)=O)CC2(F)F)nn2ccc(-c3ccc4nccn4n3)c12]

(R)-1-(3,3-Difluoro-4-((5-(imidazo[1,2-b]pyridazin-6-yl)-4-(methylamino) pyrrolo[2,1-f][1,2,4]triazin-2-yl)amino)piperidin-1-yl)ethan-1-one 46.

Yellow solid (11 mg, 0.025 mmol, 82.9% yield). ¹H NMR (499 MHz, DMSO-d₆) δ ppm 1.56-1.81 (1H, m), 1.83-1.98 (1H, m), 2.07 (3H, s), 2.94-3.06 (1H, m), 3.15 (3H, d, J=4.65 Hz), 3.58-3.92 (1H, m), 4.07-4.29 (1H, m), 4.41-4.61 (2H, m), 6.53 (1H, d, J=3.83 Hz), 7.25 (1H, d, J=3.01 Hz), 7.46 (1H, d, J=3.01 Hz), 7.76 (1H, d, J=1.10 Hz), 7.82 (1H, d, J=9.86 Hz), 8.14 (1H, d, J=9.86 Hz), 8.43 (1H, s), 9.99 (1H, q, J=4.38 Hz); ESIMS found for C₂₀H₂₁F₂N₉O m/z 442.15 (M+1).

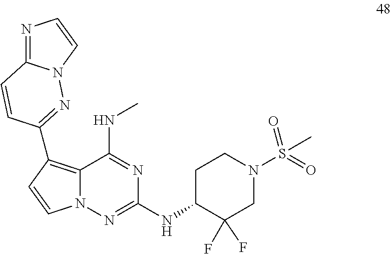 [SMILES: CNc1nc(N[C@@H]2CCN(S(C)(=O)=O)CC2(F)F)nn2ccc(-c3ccc4nccn4n3)c12]

(R)—N2-(3,3-Difluoro-1-(methylsulfonyl)piperidin-4-yl)-5-(imidazo[1,2-b]pyridazin-6-yl)-N4-methylpyrrolo[2,1-f][1,2,4]triazine-2,4-diamine 48.

Yellow solid (7 mg, 0.015 mmol, 58.6% yield). ¹H NMR (499 MHz, DMSO-d₆) δ ppm 1.79-1.92 (1H, m), 1.94-2.04 (1H, m), 2.98 (3H, s), 3.11-3.19 (1H, m), 3.16 (3H, d, J=4.65 Hz), 3.44 (1H, dd, J=25.80, 12.05 Hz), 3.53-3.64 (1H, m), 3.72-3.83 (1H, m), 4.40-4.57 (1H, m), 6.60 (1H, d, J=9.58 Hz), 7.25 (1H, d, J=3.01 Hz), 7.45 (1H, d, J=3.01 Hz), 7.76 (1H, s), 7.82 (1H, d, J=9.86 Hz), 8.14 (1H, d, J=9.86 Hz), 8.43 (1H, s), 10.00 (1H, q, J=4.47 Hz); ESIMS found for C₁₉H₂₁F₂N₉O₂S m/z 478.2 (M+1).

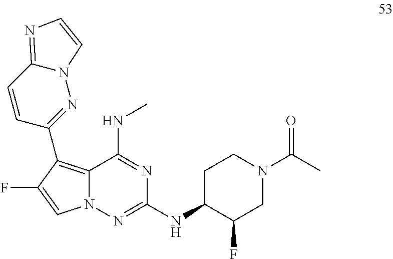 [SMILES: CNc1nc(N[C@H]2CCN(C(C)=O)C[C@H]2F)nn2cc(F)c(-c3ccc4nccn4n3)c12]

1-((3R,4S)-3-Fluoro-4-((6-fluoro-5-(imidazo[1,2-b]pyridazin-6-yl)-4-(methylamino)pyrrolo[2,1-f][1,2,4]triazin-2-yl)amino)piperidin-1-yl)ethan-1-one 53.

Pale yellow solid (28 mg, 0.063 mmol, 79.2% yield). ¹H NMR (499 MHz, DMSO-d₆) δ ppm 1.61-1.71 (1H, m), 1.72-1.88 (1H, m), 1.99 (3H, s), 2.59-2.92 (1H, m), 3.07 (3H, d, J=4.93 Hz), 3.12-3.23 (1H, m), 3.83-3.99 (1H, m), 4.01-4.16 (1H, m), 4.39-4.73 (1H, m), 4.94 (1H, d, J=49.35 Hz), 6.40 (1H, d, J=7.94 Hz), 7.59 (1H, d, J=9.31 Hz), 7.65 (1H, d, J=2.19 Hz), 7.81 (1H, d, J=1.09 Hz), 8.21 (1H, d, J=9.86 Hz), 8.47 (1H, s), 9.13 (1H, q, J=4.65 Hz); ESIMS found for C₂₀H₂₁F₂N₉O m/z 442.1 (M+1).

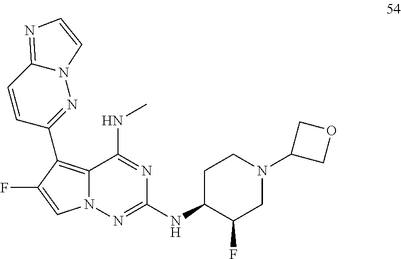 [SMILES: CNc1nc(N[C@H]2CCN(C3COC3)C[C@H]2F)nn2cc(F)c(-c3ccc4nccn4n3)c12]

6-Fluoro-N²-((3R,4S)-3-fluoro-1-(oxetan-3-yl)piperidin-4-yl)-5-(imidazo [1,2-b]pyridazin-6-yl)-N⁴-methylpyrrolo[2,1-f][1,2,4]triazine-2,4-diamine 54.

Orange solid (23 mg, 0.051 mmol, 80.7% yield). ¹H NMR (499 MHz, DMSO-d₆) δ ppm 1.64-1.75 (1H, m), 1.88 (1H, qd, J=12.14, 3.56 Hz), 1.95-2.04 (1H, m), 2.12 (1H, dd, J=37.60, 12.59 Hz), 2.69-2.80 (1H, m), 2.90-3.02 (1H, m), 3.07 (3H, d, J=4.65 Hz), 3.48 (1H, quin, J=6.30 Hz), 3.70-3.89 (1H, m), 4.39 (1H, t, J=6.16 Hz), 4.45 (1H, t, J=6.16 Hz), 4.53 (2H, td, J=6.50, 3.15 Hz), 4.89 (1H, d, J=49.65 Hz), 6.28 (1H, d, J=7.94 Hz), 7.59 (1H, d, J=9.58 Hz), 7.64 (1H, d, J=2.19 Hz), 7.81 (1H, d, J=1.10 Hz), 8.21 (1H, d, J=9.58 Hz), 8.46 (1H, s), 9.12 (1H, q, J=4.65 Hz); ESIMS found for C₂₁H₂₃F₂N₉O m/z 456.2 (M+1).

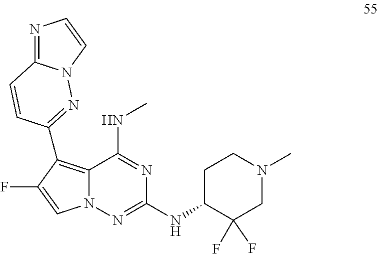 [SMILES: CNc1nc(N[C@@H]2CCN(C)CC2(F)F)nn2cc(F)c(-c3ccc4nccn4n3)c12]

(R)—N²-(3,3-Difluoro-1-methylpiperidin-4-yl)-6-fluoro-5-(imidazo[1,2-b]pyridazin-6-yl)-N⁴-methylpyrrolo[2,1-f][1,2,4]triazine-2,4-diamine 55.

Pale yellow solid (14 mg, 0.033 mmol, 90.3% yield). ¹H NMR (499 MHz, DMSO-d₆) δ ppm 1.71-1.90 (2H, m), 2.14 (1H, td, J=11.23, 2.74 Hz), 2.25 (3H, s), 2.34 (1H, ddd, J=26.85, 11.77, 1.65 Hz), 2.32 (1H, br d, J=11.77 Hz), 2.77 (1H, br d, J=11.77 Hz), 3.00-3.06 (1H, m), 3.08 (3H, d, J=4.93 Hz), 4.12-4.31 (1H, m), 6.44 (1H, br d, J=9.31 Hz), 7.59 (1H, d, J=9.58 Hz), 7.66 (1H, d, J=2.19 Hz), 7.81 (1H, s), 8.21 (1H, d, J=9.86 Hz), 8.46 (1H, s), 9.14 (1H, q, J=4.65 Hz); ESIMS found for C₁₉H₂₀F₃N₉ m/z 432.2 (M+1).

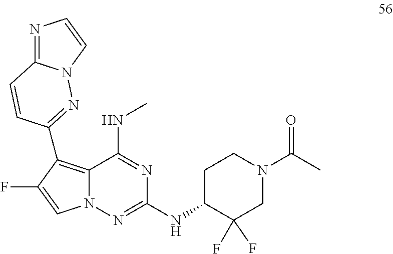 [SMILES: CNc1nc(N[C@@H]2CCN(C(C)=O)CC2(F)F)nn2cc(F)c(-c3ccc4nccn4n3)c12]

(R)-1-(3,3-Difluoro-4-((6-fluoro-5-(imidazo[1,2-b]pyridazin-6-yl)-4-(methylamino)pyrrolo[2,1-f][1,2,4]triazin-2-yl)amino)piperidin-1-yl)ethan-1-one 56.

Off-white solid (15 mg, 0.033 mmol, 90.9% yield). ¹H NMR (499 MHz, DMSO-d₆) δ ppm 1.54-1.78 (1H, m), 1.81-1.99 (1H, m), 2.06 (3H, s), 2.98 (1H, br t, J=11.23 Hz), 3.09 (3H, d, J=4.65 Hz), 3.66-3.91 (1H, m), 4.08-4.26 (1H, m), 4.42-4.59 (2H, m), 6.64 (1H, br d, J=9.58 Hz), 7.60 (1H, d, J=9.58 Hz), 7.65 (1H, d, J=2.19 Hz), 7.82 (1H, d, J=1.10 Hz), 8.22 (1H, d, J=9.58 Hz), 8.47 (1H, s), 9.16 (1H, q, J=4.75 Hz); ESIMS found for C₂₀H₂₀F₃N₉O m/z 460.15 (M+1).

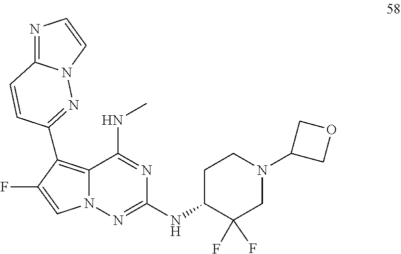 [SMILES: CNc1nc(N[C@@H]2CCN(C3COC3)CC2(F)F)nn2cc(F)c(-c3ccc4nccn4n3)c12]

(R)—N²-(3,3-Difluoro-1-(oxetan-3-yl)piperidin-4-yl)-6-fluoro-5-(imidazo[1,2-b]pyridazin-6-yl)-N⁴-methylpyrrolo[2,1-f][1,2,4]triazine-2,4-diamine 58.

Yellow solid (15 mg, 0.032 mmol, 88.2% yield). ¹H NMR (499 MHz, DMSO-d₆) δ ppm 1.71-1.83 (1H, m), 1.84-1.92 (1H, m), 2.07-2.19 (1H, m), 2.37 (1H, ddd, J=26.05, 11.50, 1.65 Hz), 2.75 (1H, br d, J=11.50 Hz), 2.94-3.04 (1H, m), 3.08 (3H, d, J=4.65 Hz), 3.59 (1H, quin, J=6.30 Hz), 4.16-4.35 (1H, m), 4.43 (2H, dt, J=14.78, 6.16 Hz), 4.54 (2H, td, J=6.57, 3.56 Hz), 6.52 (1H, d, J=9.58 Hz), 7.59 (1H, d, J=9.58 Hz), 7.66 (1H, d, J=2.19 Hz), 7.81 (1H, d, J=1.09 Hz), 8.21 (1H, d, J=9.58 Hz), 8.46 (1H, s), 9.14 (1H, q, J=4.65 Hz); ESIMS found for C₂₁H₂₂F₃N₉O m/z 474.2 (M+1).

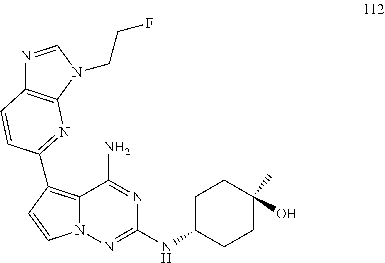 [SMILES: C[C@]1(O)CC[C@H](Nc2nc(N)c3c(-c4ccc5ncn(CCF)c5n4)ccn3n2)CC1]

(1r,4r)-4-((4-Amino-5-(3-(2-fluoroethyl)-3H-imidazo[4,5-b]pyridin-5-yl) pyrrolo[2,1-f][1,2,4]triazin-2-yl)amino)-1-methylcyclohexan-1-ol 112.

Fluffy pale yellow solid (6 mg, 0.014 mmol, 41.8% yield). ¹H NMR (499 MHz, DMSO-d₆) δ ppm 1.13 (3H, s), 1.34-1.48 (4H, m), 1.53-1.63 (2H, m), 1.77-1.89 (2H, m), 3.61 (1H, br d, J=3.83 Hz), 4.22 (1H, s), 4.58 (2H, dt, J=27.70, 4.65 Hz), 4.82 (2H, dt, J=47.15, 4.65 Hz), 5.78 (1H, d, J=8.21 Hz), 7.12 (1H, d, J=3.01 Hz), 7.39 (1H, d, J=3.01 Hz), 7.47 (1H, br s), 7.86 (1H, d, J=8.76 Hz), 8.12 (1H, d, J=8.76 Hz), 8.41 (1H, s), 10.68 (1H, br s); ESIMS found for C₂₁H₂₅FN₈O m/z 425.2 (M+1).

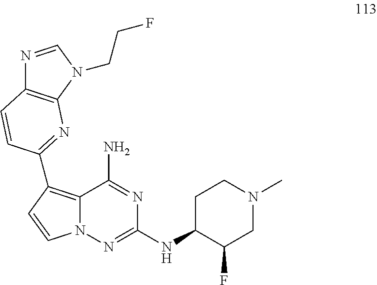 [SMILES: CN1CC[C@H](Nc2nc(N)c3c(-c4ccc5ncn(CCF)c5n4)ccn3n2)[C@H](F)C1]

N²-((3R,4S)-3-Fluoro-1-methylpiperidin-4-yl)-5-(3-(2-fluoroethyl)-3H-imidazo[4,5-b]pyridin-5-yl)pyrrolo[2,1-f][1,2,4]triazine-2,4-diamine 113.

White solid (6 mg, 0.014 mmol, 28.0% yield). ¹H NMR (499 MHz, DMSO-d₆) δ ppm 1.60-1.71 (1H, m), 1.86 (1H, qd, J=12.27, 3.97 Hz), 1.97-2.06 (1H, m), 2.07-2.21 (1H, m), 2.18 (3H, s), 2.78 (1H, br d, J=11.23 Hz), 2.98-3.09 (1H, m), 3.64-3.84 (1H, m), 4.58 (2H, dt, J=27.70, 4.65 Hz), 4.82 (2H, dt, J=46.90, 4.65 Hz), 4.85 (1H, d, J=50.20 Hz), 5.80 (1H, d, J=8.21 Hz), 7.15 (1H, d, J=2.74 Hz), 7.40 (1H, d, J=2.74 Hz), 7.61 (1H, br s), 7.87 (1H, d, J=8.76 Hz), 8.13 (1H, d, J=8.76 Hz), 8.42 (1H, s), 10.76 (1H, br s); ESIMS found for C₂₀H₂₃F₂N₉ m/z 428.25 (M+1).

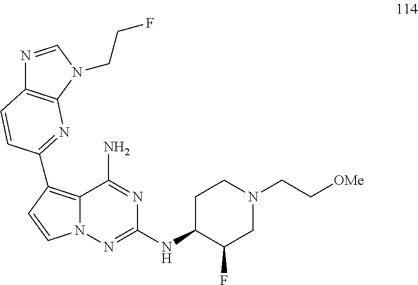 [SMILES: COCCN1CC[C@H](Nc2nc(N)c3c(-c4ccc5ncn(CCF)c5n4)ccn3n2)[C@H](F)C1]

N²-((3R,4S)-3-Fluoro-1-(2-methoxyethyl)piperidin-4-yl)-5-(3-(2-fluoroethyl)-3H-imidazo[4,5-b]pyridin-5-yl)pyrrolo[2,1-f][1,2,4]triazine-2,4-diamine 114.

White solid (10 mg, 0.021 mmol, 24.2% yield). ¹H NMR (499 MHz, DMSO-d₆) δ ppm 1.59-1.71 (1H, m), 1.83 (1H, qd, J=12.27, 3.70 Hz), 2.15 (1H, br t, J=10.81 Hz), 2.29 (1H, dd, J=38.15, 12.59 Hz), 2.51-2.53 (2H, m), 2.89 (1H, br d, J=11.22 Hz), 3.14 (1H, br t, J=10.54 Hz), 3.24 (3H, s), 3.43 (2H, t, J=5.75 Hz), 3.68-3.85 (1H, m), 4.58 (2H, dt, J=27.70, 4.65 Hz), 4.82 (2H, dt, J=46.90, 4.65 Hz), 4.84 (1H, d, J=49.90 Hz), 5.80 (1H, d, J=8.21 Hz), 7.15 (1H, d, J=3.01 Hz), 7.40 (1H, d, J=3.01 Hz), 7.60 (1H, br s), 7.87 (1H, d, J=8.76 Hz), 8.13 (1H, d, J=8.49 Hz), 8.42 (1H, s), 10.76 (1H, br s); ESIMS found for C₂₂H₂₇F₂N₉O m/z 472.2

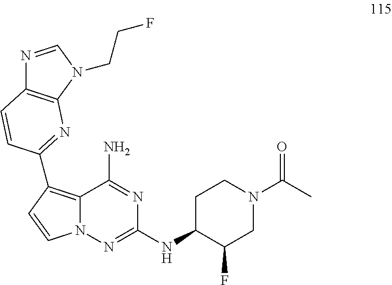 [SMILES: CC(=O)N1CC[C@H](Nc2nc(N)c3c(-c4ccc5ncn(CCF)c5n4)ccn3n2)[C@H](F)C1]

1-((3R,4S)-4-((4-Amino-5-(3-(2-fluoroethyl)-3H-imidazo[4,5-b]pyridin-5-yl) pyrrolo[2,1-f][1,2,4]triazin-2-yl)amino)-3-fluoropiperidin-1-yl)ethan-1-one 115.

White solid (8 mg, 0.018 mmol, 19.1% yield). ¹H NMR (499 MHz, DMSO-d₆) δ ppm 1.57-1.85 (2H, m), 1.96-2.06 (3H, m), 2.60-2.97 (2H, m), 3.81-3.97 (1H, m), 3.99-4.18 (1H, m), 4.37-4.51 (1H, m), 4.59 (2H, dt, J=27.70, 4.65 Hz), 4.83 (2H, dt, J=47.15, 4.65 Hz), 4.95 (1H, d, J=48.80 Hz), 6.00 (1H, d, J=4.93 Hz), 7.16 (1H, d, J=2.74 Hz), 7.40 (1H, d, J=3.01 Hz), 7.61 (1H, br s), 7.88 (1H, d, J=8.76 Hz), 8.13 (1H, d, J=8.49 Hz), 8.42 (1H, s), 10.78 (1H, br s); ESIMS found for C₂₁H₂₃F₂N₉O m/z 456.1 (M+1).

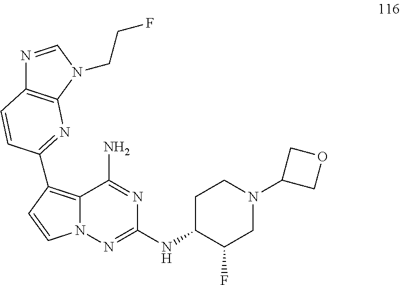 [SMILES: Nc1nc(N[C@@H]2CCN(C3COC3)C[C@@H]2F)nn2ccc(-c3ccc4ncn(CCF)c4n3)c12]

N²-((3S,4R)-3-Fluoro-1-(oxetan-3-yl)piperidin-4-yl)-5-(3-(2-fluoroethyl)-3H-imidazo[4,5-b]pyridin-5-yl)pyrrolo[2,1-f][1,2,4]triazine-2,4-diamine 116.

White solid (17.4 mg, 0.037 mmol, 42.1% yield). ¹H NMR (499 MHz, DMSO-d₆) δ ppm 1.65-1.75 (1H, m), 1.85 (1H, qd, J=12.14, 3.56 Hz), 1.93-2.02 (1H, m), 2.12 (1H, dd, J=37.30, 12.59 Hz), 2.69-2.79 (1H, m), 2.91-3.03 (1H, m), 3.48 (1H, quin, J=6.43 Hz), 3.72-3.90 (1H, m), 4.39 (1H, t, J=6.30 Hz), 4.45 (1H, t, J=6.02 Hz), 4.53 (2H, td, J=6.57, 2.74 Hz), 4.58 (2H, dt, J=28.00, 4.65 Hz), 4.82 (2H, dt, J=47.20, 4.65 Hz), 4.88 (1H, d, J=49.35 Hz), 5.88 (1H, d, J=8.21 Hz), 7.15 (1H, d, J=3.29 Hz), 7.39 (1H, d, J=3.29 Hz), 7.60 (1H, br s), 7.87 (1H, d, J=8.76 Hz), 8.13 (1H, d, J=8.21 Hz), 10.76 (1H, br s); ESIMS found for C₂₂H₂₅F₂N₉O m/z 470.15 (M+1).

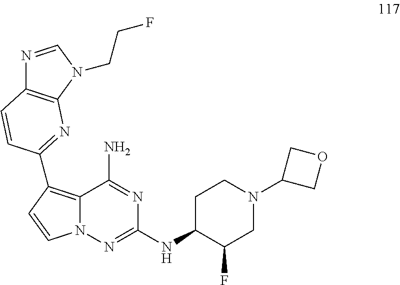 [SMILES: Nc1nc(N[C@H]2CCN(C3COC3)C[C@H]2F)nn2ccc(-c3ccc4ncn(CCF)c4n3)c12]

N²-((3R,4S)-3-Fluoro-1-(oxetan-3-yl)piperidin-4-yl)-5-(3-(2-fluoroethyl)-3H-imidazo[4,5-b]pyridin-5-yl)pyrrolo[2,1-f][1,2,4]triazine-2,4-diamine 117.

Off-white solid (19.8 mg, 0.042 mmol, 47.9% yield). ¹H NMR (499 MHz, DMSO-d₆) δ ppm 1.70 (1H, br d, J=9.86 Hz), 1.85 (1H, qd, J=11.86, 2.74 Hz), 1.93-2.01 (1H, m), 2.11 (1H, dd, J=37.30, 12.59 Hz), 2.73 (1H, br d, J=10.40 Hz), 2.96 (1H, br t, J=10.40 Hz), 3.47 (1H, quin, J=6.05 Hz), 3.71-3.88 (1H, m), 4.39 (1H, br t, J=6.02 Hz), 4.45 (1H, br t, J=6.02 Hz), 4.48-4.55 (2H, m), 4.55-4.65 (2H, m), 4.83 (2H, dt, J=47.20, 4.28 Hz), 4.88 (1H, d, J=49.35 Hz), 5.88 (1H, br d, J=8.21 Hz), 7.14 (1H, d, J=2.74 Hz), 7.39 (1H, d, J=2.19 Hz), 7.61 (1H, br s), 7.87 (1H, br d, J=8.21 Hz), 8.14 (1H, br d, J=7.67 Hz), 10.77 (1H, br s); ESIMS found for C₂₂H₂₅F₂N₉O m/z 470.2 (M+1).

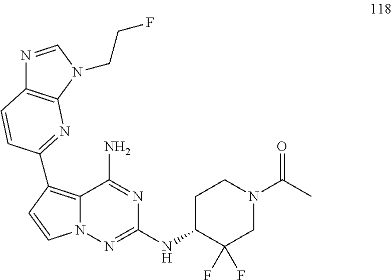 [SMILES: CC(=O)N1CC[C@@H](Nc2nc(N)c3c(-c4ccc5ncn(CCF)c5n4)ccn3n2)C(F)(F)C1]

(R)-1-(4-((4-Amino-5-(3-(2-fluoroethyl)-3H-imidazo[4,5-b]pyridin-5-yl) pyrrolo[2,1-f][1,2,4]triazin-2-yl)amino)-3,3-difluoropiperidin-1-yl)ethan-1-one 118.

Fluffy pale-yellow solid (26 mg, 0.055 mmol, 47.3% yield). ¹H NMR (499 MHz, DMSO-d₆) δ ppm 1.52-1.78 (1H, m), 1.80-1.97 (1H, m), 2.02-2.08 (3H, m), 2.91-3.02 (1H, m), 3.63-3.89 (1H, m), 4.08-4.28 (1H, m), 4.40-4.54 (2H, m), 4.59 (2H, dt, J=27.70, 4.65 Hz), 4.83 (2H, dt, J=47.15, 4.65 Hz), 6.29 (1H, br d, J=6.02 Hz), 7.17 (1H, d, J=3.01 Hz), 7.41 (1H, d, J=1.37 Hz), 7.64 (1H, br s), 7.88 (1H, d, J=8.76 Hz), 8.14 (1H, d, J=8.76 Hz), 8.42 (1H, s), 10.82 (1H, br s); ESIMS found for C₂₁H₂₂F₃N₉O m/z 474.2 (M+1).

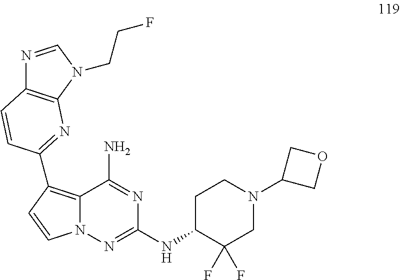 [SMILES: Nc1nc(N[C@@H]2CCN(C3COC3)CC2(F)F)nn2ccc(-c3ccc4ncn(CCF)c4n3)c12]

(R)—N²-(3,3-Difluoro-1-(oxetan-3-yl)piperidin-4-yl)-5-(3-(2-fluoroethyl)-3H-imidazo[4,5-b]pyridin-5-yl)pyrrolo[2,1-f][1,2,4]triazine-2,4-diamine 119.

White solid (5 mg, 0.010 mmol, 12.3% yield). ¹H NMR (499 MHz, DMSO-d₆) δ ppm 1.66-1.82 (1H, m), 1.86 (1H, br dd, J=8.49, 4.11 Hz), 2.11 (1H, br t, J=10.68 Hz), 2.35 (1H, dd, J=26.30, 11.77 Hz), 2.74 (1H, br d, J=11.23 Hz), 2.94-3.07 (1H, m), 3.59 (1H, quin, J=6.37 Hz), 4.17-4.36 (1H, m), 4.43 (2H, dt, J=13.83, 6.23 Hz), 4.54 (2H, dt, J=6.60, 3.60 Hz), 4.59 (2H, dt, J=27.60, 4.65 Hz), 4.83 (2H, dt, J=47.15, 4.65 Hz), 6.16 (1H, br d, J=9.31 Hz), 7.16 (1H, d, J=3.01 Hz), 7.40 (1H, d, J=2.74 Hz), 7.64 (1H, br s), 7.88 (1H, d, J=8.76 Hz), 8.13 (1H, d, J=8.76 Hz), 8.42 (1H, s), 10.81 (1H, br s); ESIMS found for C₂₂H₂₄F₃N₉O m/z 488.2 (M+1).

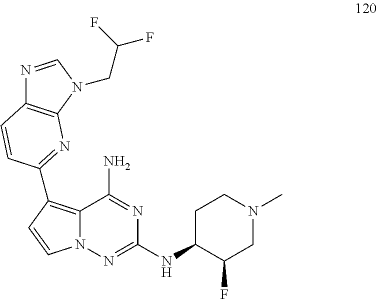 [SMILES: CN1CC[C@H](Nc2nc(N)c3c(-c4ccc5ncn(CC(F)F)c5n4)ccn3n2)[C@H](F)C1]

5-(3-(2,2-Difluoroethyl)-3H-imidazo[4,5-b]pyridin-5-yl)-N²-((3R,4S)-3-fluoro-1-methylpiperidin-4-yl)pyrrolo[2,1-f][1,2,4]triazine-2,4-diamine 120.

White solid (7 mg, 0.016 mmol, 31.3% yield). ¹H NMR (499 MHz, DMSO-d₆) δ ppm 1.61-1.71 (1H, m), 1.85 (1H, qd, J=12.18, 3.70 Hz), 2.02 (1H, br t, J=11.09 Hz), 2.08-2.20 (1H, m), 2.18 (2H, s), 2.78 (1H, br d, J=10.95 Hz), 2.98-3.09 (1H, m), 3.62-3.83 (1H, m), 4.74 (2H, td, J=16.50, 2.05 Hz), 4.85 (1H, d, J=49.95 Hz), 5.80 (1H, d, J=7.94 Hz), 6.48 (1H, tt, J=54.30, 2.70 Hz), 7.14 (1H, d, J=2.74 Hz), 7.40 (1H, d, J=2.74 Hz), 7.59 (1H, br s), 7.88 (1H, d, J=8.76 Hz), 8.15 (1H, d, J=8.49 Hz), 8.40 (1H, s), 10.57 (1H, br s); ESIMS found for C₂₀H₂₂F₃N₉ m/z 446.2 (M+1).

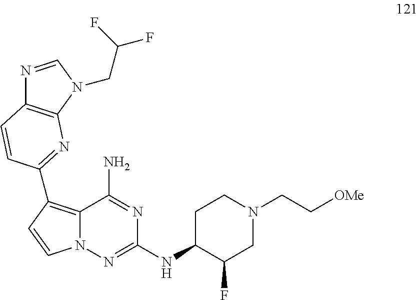 [SMILES: COCCN1CC[C@H](Nc2nc(N)c3c(-c4ccc5ncn(CC(F)F)c5n4)ccn3n2)[C@H](F)C1]

5-(3-(2,2-Difluoroethyl)-3H-imidazo[4,5-b]pyridin-5-yl)-N²-((3R,4S)-3-fluoro-1-(2-methoxyethyl)piperidin-4-yl)pyrrolo[2,1-f][1,2,4]triazine-2,4-diamine 121.

White solid (22 mg, 0.045 mmol, 51.4% yield). ¹H NMR (499 MHz, DMSO-d₆) δ ppm 1.59-1.71 (1H, m), 1.83 (1H, qd, J=12.18, 3.70 Hz), 2.15 (1H, br t, J=11.09 Hz), 2.29 (1H, dd, J=38.10, 12.59 Hz), 2.51 (2H, t, J=5.75 Hz), 2.82-2.94 (1H, m), 3.08-3.18 (1H, m), 3.24 (3H, s), 3.43 (2H, t, J=5.75 Hz), 3.68-3.85 (1H, m), 4.74 (2H, td, J=16.63, 2.05 Hz), 4.84 (1H, d, J=50.20 Hz), 5.79 (1H, d, J=8.21 Hz), 6.48 (1H, tt, J=54.30, 2.46 Hz), 7.14 (1H, d, J=3.01 Hz), 7.40 (1H, d, J=2.74 Hz), 7.59 (1H, br s), 7.88 (1H, d, J=8.76 Hz), 8.15 (1H, d, J=8.49 Hz), 8.40 (1H, s), 10.57 (1H, br s); ESIMS found for C₂₂H₂₆F₃N₉O m/z 490.2 (M+1).

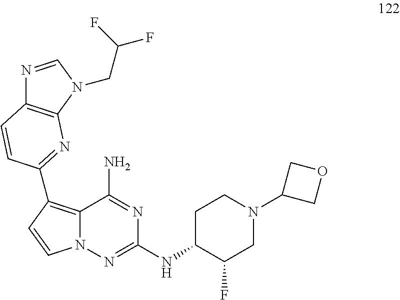 [SMILES: Nc1nc(N[C@@H]2CCN(C3COC3)C[C@@H]2F)nn2ccc(-c3ccc4ncn(CC(F)F)c4n3)c12]

5-(3-(2,2-Difluoroethyl)-3H-imidazo[4,5-b]pyridin-5-yl)-N²-((3S,4R)-3-fluoro-1-(oxetan-3-yl)piperidin-4-yl)pyrrolo[2,1-f][1,2,4]triazine-2,4-diamine 122.

Off-white solid (15 mg, 0.031 mmol, 35.0% yield). ¹H NMR (499 MHz, DMSO-d₆) δ ppm 1.64-1.76 (1H, m), 1.85 (1H, qd, J=12.18, 3.70 Hz), 1.94-2.02 (1H, m), 2.12 (1H, dd, J=37.60, 12.59 Hz), 2.74 (1H, br d, J=11.23 Hz), 2.97 (1H, br t, J=10.13 Hz), 3.48 (1H, quin, J=6.37 Hz), 3.70-3.90 (1H, m), 4.39 (1H, t, J=6.02 Hz), 4.45 (1H, t, J=6.02 Hz), 4.54 (2H, td, J=6.43, 2.74 Hz), 4.75 (2H, td, J=16.70, 2.19 Hz), 4.88 (1H, d, J=49.95 Hz), 5.88 (1H, d, J=8.21 Hz), 6.48 (1H, tt, J=54.30, 2.47 Hz), 7.14 (1H, d, J=3.01 Hz), 7.40 (1H, d, J=2.74 Hz), 7.58 (1H, br s), 7.88 (1H, d, J=8.76 Hz), 8.15 (1H, d, J=8.49 Hz), 8.41 (1H, s), 10.58 (1H, br s); ESIMS found for C₂₂H₂₄F₃N₉O m/z 488.2 (M+1).

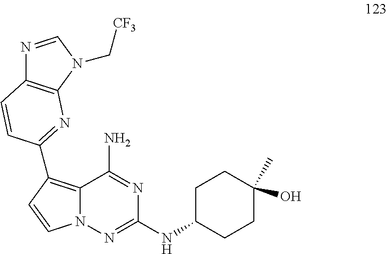 [SMILES: C[C@]1(O)CC[C@H](Nc2nc(N)c3c(-c4ccc5ncn(CC(F)(F)F)c5n4)ccn3n2)CC1]

(1r,4r)-4-((4-Amino-5-(3-(2,2,2-trifluoroethyl)-3H-imidazo[4,5-b]pyridin-5-yl)pyrrolo[2,1-f][1,2,4]triazin-2-yl)amino)-1-methylcyclohexan-1-ol 123.

Off-white solid (1 mg, 0.002 mmol, 9.2% yield). ¹H NMR (499 MHz, DMSO-d₆) δ ppm 1.14 (3H, s), 1.34-1.47 (4H, m), 1.53-1.62 (2H, m), 1.78-1.88 (2H, m), 3.58-3.69 (1H, m), 4.22 (1H, br s), 5.20 (2H, q, J=9.22 Hz), 5.77 (1H, d, J=8.21 Hz), 7.13 (1H, d, J=2.74 Hz), 7.40 (1H, d, J=2.74 Hz), 7.53 (1H, br s), 7.91 (1H, d, J=8.76 Hz), 8.17 (1H, d, J=8.49 Hz), 8.47 (1H, s), 10.36 (1H, br s); ESIMS found for C₂₁H₂₃F₃N₈O m/z 461.2 (M+1).

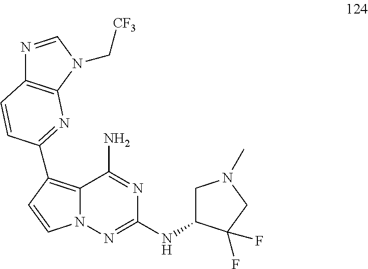 [SMILES: CN1C[C@@H](Nc2nc(N)c3c(-c4ccc5ncn(CC(F)(F)F)c5n4)ccn3n2)C(F)(F)C1]

(R)—N²-(4,4-Difluoro-1-methylpyrrolidin-3-yl)-5-(3-(2,2,2-trifluoroethyl)-3H-imidazo[4,5-b]pyridin-5-yl)pyrrolo[2,1-f][1,2,4]triazine-2,4-diamine 124.

Off-white solid (20 mg, 0.043 mmol, 66.6% yield). ¹H NMR (499 MHz, DMSO-d₆) δ ppm 2.27 (3H, s), 2.38 (1H, t, J=9.03 Hz), 2.51-2.62 (1H, m), 3.17-3.24 (2H, m), 4.53-4.67 (1H, m), 5.21 (2H, q, J=9.22 Hz), 6.31 (1H, d, J=9.31 Hz), 7.18 (1H, d, J=3.01 Hz), 7.42 (1H, d, J=2.74 Hz), 7.73 (1H, br s), 7.94 (1H, d, J=8.76 Hz), 8.19 (1H, d, J=8.49 Hz), 8.48 (1H, s), 10.49 (1H, br s); ESIMS found for C₁₉H₁₈F₅N₉ m/z 468.1 (M+1).

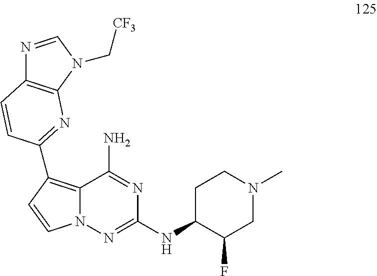 [SMILES: CN1CC[C@H](Nc2nc(N)c3c(-c4ccc5ncn(CC(F)(F)F)c5n4)ccn3n2)[C@H](F)C1]

N²-((3R,4S)-3-Fluoro-1-methylpiperidin-4-yl)-5-(3-(2,2,2-trifluoroethyl)-3H-imidazo[4,5-b]pyridin-5-yl)pyrrolo[2,1-f][1,2,4]triazine-2,4-diamine 125.

White solid (6 mg, 0.013 mmol, 25.8% yield). ¹H NMR (499 MHz, DMSO-d₆) δ ppm 1.61-1.72 (1H, m), 1.86 (1H, qd, J=12.14, 3.56 Hz), 2.04 (1H, br t, J=10.81 Hz), 2.16 (1H, dd, J=37.55, 13.14 Hz), 2.19 (1H, s), 2.79 (1H, br d, J=10.95 Hz), 3.00-3.09 (1H, m), 3.67-3.80 (1H, m), 4.86 (1H, d, J=49.65 Hz), 5.20 (2H, q, J=9.13 Hz), 5.81 (1H, d, J=8.21 Hz), 7.16 (1H, d, J=2.74 Hz), 7.41 (1H, d, J=2.74 Hz), 7.68 (1H, br s), 7.93 (1H, d, J=8.76 Hz), 8.18 (1H, d, J=8.49 Hz), 8.48 (1H, s), 10.44 (1H, br s); ESIMS found for C₂₀H₂₁F₄N₉ m/z 464.15 (M+1).

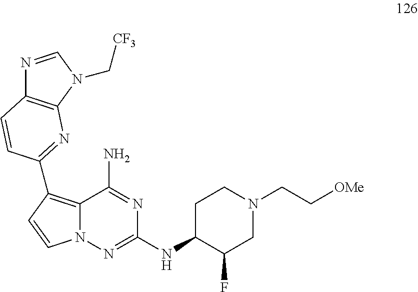 [SMILES: COCCN1CC[C@H](Nc2nc(N)c3c(-c4ccc5ncn(CC(F)(F)F)c5n4)ccn3n2)[C@H](F)C1]

N²-((3R,4S)-3-Fluoro-1-(2-methoxyethyl)piperidin-4-yl)-5-(3-(2,2,2-trifluoroethyl)-3H-imidazo[4,5-b]pyridin-5-yl)pyrrolo[2,1-f][1,2,4]triazine-2,4-diamine 126.

Beige solid (23 mg, 0.045 mmol, 51.8% yield). ¹H NMR (499 MHz, DMSO-d₆) δ ppm 1.61-1.71 (1H, m), 1.84 (1H, qd, J=12.23, 3.83 Hz), 2.16 (1H, br t, J=11.09 Hz), 2.30 (1H, dd, J=38.10, 12.59 Hz), 2.52 (2H, t, J=5.89 Hz), 2.90 (1H, br d, J=11.50 Hz), 3.10-3.18 (1H, m), 3.24 (3H, s), 3.43 (2H, br t, J=5.89 Hz), 3.69-3.84 (1H, m), 4.84 (1H, d, J=49.90 Hz), 5.20 (2H, q, J=9.03 Hz), 5.80 (1H, d, J=8.21 Hz), 7.15 (1H, d, J=3.01 Hz), 7.41 (1H, d, J=2.74 Hz), 7.67 (1H, br s), 7.92 (1H, d, J=8.76 Hz), 8.18 (1H, d, J=8.76 Hz), 8.47 (1H, s), 10.44 (1H, br s); ESIMS found for C₂₂H₂₅F₄N₉O m/z 508.2 (M+1).

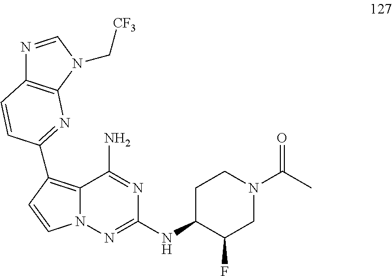 [SMILES: CC(=O)N1CC[C@H](Nc2nc(N)c3c(-c4ccc5ncn(CC(F)(F)F)c5n4)ccn3n2)[C@H](F)C1]

1-((3R,4S)-4-((4-Amino-5-(3-(2,2,2-trifluoroethyl)-3H-imidazo[4,5-b]pyridin-5-yl)pyrrolo[2,1-f][1,2,4]triazin-2-yl)amino)-3-fluoropiperidin-1-yl)ethan-1-one 127.

White solid (7 mg, 0.014 mmol, 15.5% yield). ¹H NMR (499 MHz, DMSO-d₆) δ ppm 1.58-1.70 (1H, m), 1.71-1.88 (1H, m), 1.97-2.05 (3H, m), 2.58-2.95 (2H, m), 3.83-3.99 (1H, m), 4.02-4.19 (1H, m), 4.38-4.74 (1H, m), 4.95 (1H, d, J=48.85 Hz), 5.21 (2H, q, J=9.22 Hz), 6.00 (1H, d, J=4.93 Hz), 7.17 (1H, d, J=2.74 Hz), 7.41 (1H, d, J=3.01 Hz), 7.68 (1H, br s), 7.93 (1H, d, J=8.76 Hz), 8.18 (1H, d, J=8.76 Hz), 8.48 (1H, s), 10.46 (1H, br s); ESIMS found for C₂₁H₂₁F₄N₉O m/z 492.2 (M+1).

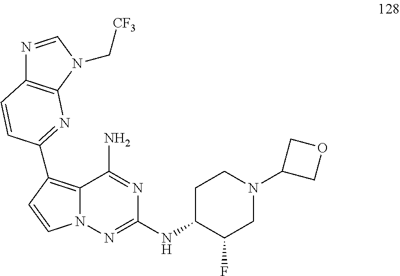 [SMILES: Nc1nc(N[C@@H]2CCN(C3COC3)C[C@@H]2F)nn2ccc(-c3ccc4ncn(CC(F)(F)F)c4n3)c12]

N²-((3S,4R)-3-Fluoro-1-(oxetan-3-yl)piperidin-4-yl)-5-(3-(2,2,2-trifluoroethyl)-3H-imidazo[4,5-b]pyridin-5-yl)pyrrolo[2,1-f][1,2,4]triazine-2,4-diamine 128.

White solid (11 mg, 0.022 mmol, 24.7% yield). ¹H NMR (499 MHz, DMSO-d₆) δ ppm 1.70 (1H, br dd, J=13.00, 3.70 Hz), 1.85 (1H, qd, J=12.14, 3.56 Hz), 1.94-2.03 (1H, m), 2.12 (1H, dd, J=37.85, 12.59 Hz), 2.74 (1H, br d, J=10.95 Hz), 2.92-3.04 (1H, m), 3.48 (1H, dt, J=12.66, 6.40 Hz), 3.71-3.89 (1H, m), 4.39 (1H, t, J=6.16 Hz), 4.45 (1H, t, J=6.16 Hz), 4.54 (2H, td, J=6.43, 2.74 Hz), 4.88 (1H, d, J=49.65 Hz), 5.21 (2H, q, J=9.22 Hz), 5.88 (1H, d, J=8.21 Hz), 7.16 (1H, d, J=3.01 Hz), 7.41 (1H, d, J=2.74 Hz), 7.67 (1H, br s), 7.92 (1H, d, J=8.76 Hz), 8.18 (1H, br d, J=8.49 Hz), 8.49 (1H, br s), 10.45 (1H, br s); ESIMS found for C₂₂H₂₃F₄N₉O m/z 506.2 (M+1).

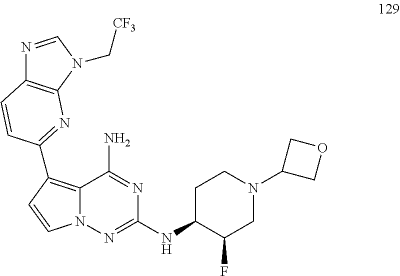 [SMILES: Nc1nc(N[C@H]2CCN(C3COC3)C[C@H]2F)nn2ccc(-c3ccc4ncn(CC(F)(F)F)c4n3)c12]

N²-((3R,4S)-3-Fluoro-1-(oxetan-3-yl)piperidin-4-yl)-5-(3-(2,2,2-trifluoroethyl)-3H-imidazo[4,5-b]pyridin-5-yl)pyrrolo[2,1-f][1,2,4]triazine-2,4-diamine 129.

White solid (12 mg, 0.024 mmol, 27.0% yield). 1.64-1.75 (1H, m), 1.85 (1H, qd, J=12.05, 3.01 Hz), 1.93-2.03 (1H, m), 2.12 (1H, dd, J=37.60, 12.59 Hz), 2.69-2.80 (1H, m), 2.91-3.03 (1H, m), 3.48 (1H, quin, J=6.30 Hz), 3.72-3.89 (1H, m), 4.39 (1H, t, J=6.16 Hz), 4.45 (1H, t, J=6.16 Hz), 4.53 (2H, td, J=6.57, 2.74 Hz), 4.88 (1H, d, J=49.65 Hz), 5.15-5.26 (2H, m), 5.88 (1H, d, J=8.21 Hz), 7.16 (1H, d, J=3.01 Hz), 7.41 (1H, d, J=2.74 Hz), 7.67 (1H, br s), 7.92 (1H, d, J=8.76 Hz), 8.18 (1H, d, J=8.76 Hz), 8.48 (1H, s), 10.45 (1H, br s); ESIMS found for C₂₂H₂₃F₄N₉O m/z 506.2 (M+1).

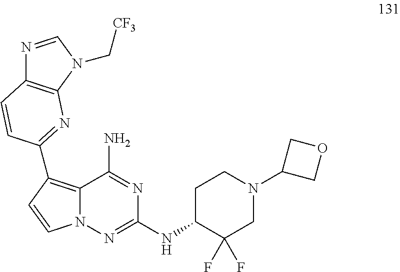 [SMILES: Nc1nc(N[C@@H]2CCN(C3COC3)CC2(F)F)nn2ccc(-c3ccc4ncn(CC(F)(F)F)c4n3)c12]

(R)—N²-(3,3-Difluoro-1-(oxetan-3-yl)piperidin-4-yl)-5-(3-(2,2,2-trifluoroethyl)-3H-imidazo[4,5-b]pyridin-5-yl)pyrrolo[2,1-f][1,2,4]triazine-2,4-diamine 131.

White solid (6 mg, 0.012 mmol, 13.7% yield). ¹H NMR (499 MHz, DMSO-d₆) δ ppm 1.68-1.81 (1H, m), 1.86 (1H, br dd, J=8.49, 4.11 Hz), 2.11 (1H, br t, J=10.81 Hz), 2.34 (1H, dd, J=26.30, 11.77 Hz), 2.74 (1H, br d, J=11.23 Hz), 2.95-3.06 (1H, m), 3.59 (1H, quin, J=6.30 Hz), 4.20-4.37 (1H, m), 4.43 (2H, dt, J=13.83, 6.25 Hz), 4.54 (2H, td, J=6.57, 3.56 Hz), 5.21 (2H, q, J=9.22 Hz), 6.16 (1H, br d, J=9.58 Hz), 7.17 (1H, d, J=3.01 Hz), 7.41 (1H, d, J=3.01 Hz), 7.71 (1H, br s), 7.93 (1H, d, J=8.49 Hz), 8.18 (1H, d, J=8.76 Hz), 8.48 (1H, s), 10.49 (1H, br s); ESIMS found for C₂₂H₂₂F₅N₉O m/z 524.15 (M+1).

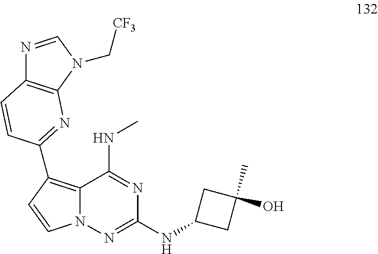 [SMILES: CNc1nc(N[C@H]2C[C@](C)(O)C2)nn2ccc(-c3ccc4ncn(CC(F)(F)F)c4n3)c12]

(1r,3r)-1-Methyl-3-((4-(methylamino)-5-(3-(2,2,2-trifluoroethyl)-3H-imidazo [4,5-b]pyridin-5-yl)pyrrolo[2,1-f][1,2,4]triazin-2-yl)amino)cyclobutan-1-ol 132.

Beige solid (22 mg, 0.049 mmol, 55.5% yield). ¹H NMR (499 MHz, DMSO-d₆) δ ppm 1.27 (3H, s), 1.91-2.00 (2H, m), 2.24-2.33 (2H, m), 3.10 (3H, d, J=4.93 Hz), 4.26 (1H, sxt, J=7.56 Hz), 4.74 (1H, s), 5.35 (2H, q, J=9.31 Hz), 6.38 (1H, d, J=7.12 Hz), 7.15 (1H, d, J=3.01 Hz), 7.38 (1H, d, J=2.74 Hz), 7.95 (1H, d, J=8.76 Hz), 8.18 (1H, d, J=8.76 Hz), 8.48 (1H, s), 10.87 (1H, q, J=4.38 Hz); ESIMS found for C₂₀H₂₁F₃N₈O m/z 447.2 (M+1).

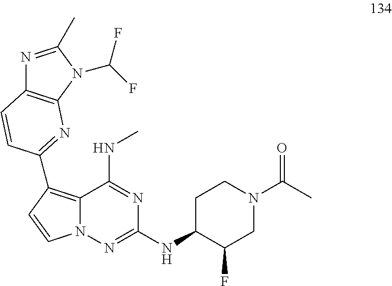 [SMILES: CNc1nc(N[C@H]2CCN(C(C)=O)C[C@H]2F)nn2ccc(-c3ccc4nc(C)n(C(F)F)c4n3)c12]

1-((3R,4S)-4-((5-(3-Ddifluoromethyl)-2-methyl-3H-imidazo[4,5-b]pyridin-5-yl)-4-(methylamino)pyrrolo[2,1-f][1,2,4]triazin-2-yl)amino)-3-fluoropiperidin-1-yl)ethan-1-one 134.

Fluffy off-white solid (5 mg, 0.010 mmol, 43.7% yield). ¹H NMR (499 MHz, DMSO-d₆) δ ppm 1.56-1.71 (1H, m), 1.73-1.88 (1H, m), 1.99 (3H, s), 2.71 (3H, s), 3.08 (3H, d, J=4.65 Hz), 3.14-3.24 (1H, m), 3.81-4.00 (1H, m), 4.02-4.18 (1H, m), 4.36-4.75 (2H, m), 4.97 (1H, d, J=49.35 Hz), 6.11 (1H, br d, J=8.21 Hz), 7.15 (1H, d, J=3.01 Hz), 7.38 (1H, d, J=2.74 Hz), 7.95 (1H, d, J=8.76 Hz), 8.09 (1H, t, J=57.05 Hz), 8.05-8.13 (1H, m), 10.78 (1H, q, J=4.56 Hz); ESIMS found for C₂₂H₂₄F₃N₉O m/z 488.2 (M+1).

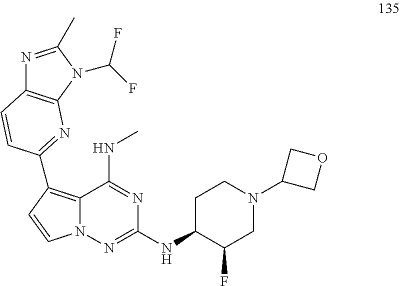 [SMILES: CNc1nc(N[C@H]2CCN(C3COC3)C[C@H]2F)nn2ccc(-c3ccc4nc(C)n(C(F)F)c4n3)c12]

5-(3-(Difluoromethyl)-2-methyl-3H-imidazo[4,5-b]pyridin-5-yl)-N²-((3R,4S)-3-fluoro-1-(oxetan-3-yl)piperidin-4-yl)-N⁴-methylpyrrolo[2,1-f][1,2,4]triazine-2,4-diamine 135.

Fluffy off-white solid (2 mg, 0.004 mmol, 20.2% yield). ¹H NMR (499 MHz, DMSO-d₆) δ ppm 1.67-1.75 (1H, m), 1.87 (1H, qd, J=12.18, 3.97 Hz), 1.96-2.04 (1H, m), 2.13 (1H, dd, J=37.77, 12.53 Hz), 2.71 (3H, s), 2.74 (1H, br d, J=12.59 Hz), 2.94-3.02 (1H, m), 3.07 (3H, d, J=4.38 Hz), 3.48 (1H, quin, J=6.37 Hz), 3.71-3.92 (1H, m), 4.40 (1H, t, J=6.16 Hz), 4.45 (1H, t, J=6.16 Hz), 4.54 (2H, td, J=6.57, 3.01 Hz), 4.90 (1H, d, J=49.60 Hz), 5.98 (1H, d, J=8.21 Hz), 7.15 (1H, d, J=3.01 Hz), 7.38 (1H, d, J=2.74 Hz), 7.94 (1H, d, J=8.76 Hz), 8.08 (1H, t, J=57.05 Hz), 8.08 (1H, d, J=8.76 Hz), 10.77 (1H, q, J=4.56 Hz); ESIMS found for C₂₃H₂₆F₃N₉O m/z 502.2 (M+1).

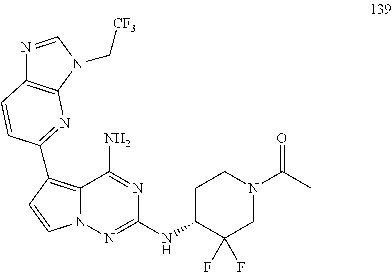 [SMILES: CC(=O)N1CC[C@@H](Nc2nc(N)c3c(-c4ccc5ncn(CC(F)(F)F)c5n4)ccn3n2)C(F)(F)C1]

(R)-1-(4-((4-Amino-5-(3-(2,2,2-trifluoroethyl)-3H-imidazo[4,5-b]pyridin-5-yl)pyrrolo[2,1-f][1,2,4]triazin-2-yl)amino)-3,3-difluoropiperidin-1-yl)ethan-1-one 139.

White solid (5 mg, 0.010 mmol, 14.1% yield). ¹H NMR (499 MHz, DMSO-d₆) δ ppm 1.52-1.80 (1H, m), 1.82-1.98 (1H, m), 2.03-2.08 (3H, m), 2.92-3.07 (1H, m), 3.58-3.92 (1H, m), 4.06-4.28 (1H, m), 4.35-4.61 (2H, m), 5.21 (2H, q, J=9.22 Hz), 6.29 (1H, br d, J=2.46 Hz), 7.18 (1H, d, J=3.01 Hz), 7.41 (1H, dd, J=2.87, 1.51 Hz), 7.71 (1H, br s), 7.94 (1H, d, J=8.76 Hz), 8.19 (1H, d, J=8.76 Hz), 8.48 (1H, s), 10.51 (1H, br s); ESIMS found for C₂₁H₂₀F₅N₉O m/z 510.2 (M+1).

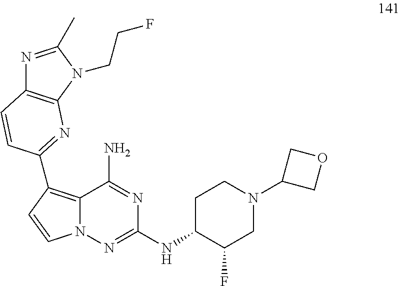 [SMILES: Cc1nc2ccc(-c3ccn4nc(N[C@@H]5CCN(C6COC6)C[C@@H]5F)nc(N)c34)nc2n1CCF]

N²-((3S,4R)-3-Fluoro-1-(oxetan-3-yl)piperidin-4-yl)-5-(3-(2-fluoroethyl)-2-methyl-3H-imidazo[4,5-b]pyridin-5-yl)pyrrolo[2,1-f][1,2,4]triazine-2,4-diamine 141.

White solid (9 mg, 0.019 mmol, 28.8% yield). ¹H NMR (499 MHz, DMSO-d₆) δ ppm 1.70 (1H, br dd, J=9.03, 4.11 Hz), 1.85 (1H, qd, J=12.14, 3.83 Hz), 1.93-2.02 (1H, m), 2.11 (1H, dd, J=37.60, 12.59 Hz), 2.59 (3H, s), 2.68-2.79 (1H, m), 2.97 (1H, br t, J=10.95 Hz), 3.48 (1H, dt, J=12.59, 6.30 Hz), 3.69-3.88 (1H, m), 4.39 (1H, t, J=6.16 Hz), 4.45 (1H, t, J=6.16 Hz), 4.50-4.56 (2H, m), 4.55 (2H, dt, J=27.70, 4.65 Hz), 4.78 (2H, dt, J=47.15, 4.65 Hz), 4.87 (1H, d, J=49.90 Hz), 5.84 (1H, d, J=8.21 Hz), 7.11 (1H, d, J=3.01 Hz), 7.38 (1H, d, J=3.01 Hz), 7.59 (1H, br s), 7.80 (1H, d, J=8.49 Hz), 7.98 (1H, br d, J=8.49 Hz), 10.80 (1H, br s); ESIMS found for C₂₃H₂₇F₂N₉O m/z 484.2 (M+1).

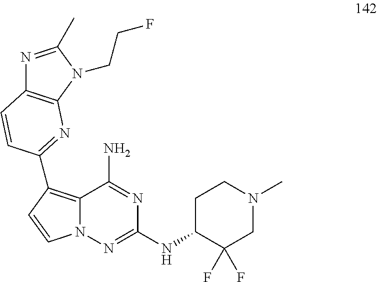 [SMILES: Cc1nc2ccc(-c3ccn4nc(N[C@@H]5CCN(C)CC5(F)F)nc(N)c34)nc2n1CCF]

(R)—N²-(3,3-Difluoro-1-methylpiperidin-4-yl)-5-(3-(2-fluoroethyl)-2-methyl-3H-imidazo[4,5-b]pyridin-5-yl)pyrrolo[2,1-f][1,2,4]triazine-2,4-diamine 142.

White solid (4 mg, 0.009 mmol, 23.0% yield). ¹H NMR (499 MHz, DMSO-d₆) δ ppm 1.69-1.88 (2H, m), 2.05-2.16 (1H, m), 2.25 (3H, s), 2.27-2.37 (1H, m), 2.59 (3H, s), 2.71-2.81 (1H, m), 3.04 (1H, td, J=9.65, 7.53 Hz), 4.14-4.31 (1H, m), 4.55 (2H, dt, J=27.70, 4.65 Hz), 4.78 (2H, dt, J=47.20, 4.65 Hz), 6.03 (1H, d, J=9.31 Hz), 7.12 (1H, d, J=3.01 Hz), 7.39 (1H, d, J=3.01 Hz), 7.64 (1H, br s), 7.81 (1H, d, J=8.49 Hz), 7.98 (1H, d, J=8.49 Hz), 10.84 (1H, br s); ESIMS found for C₂₁H₂₄F₃N₉ m/z 460.25 (M+1).

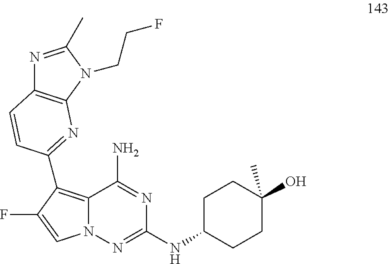 [SMILES: Cc1nc2ccc(-c3c(F)cn4nc(N[C@H]5CC[C@](C)(O)CC5)nc(N)c34)nc2n1CCF]

(1r,4r)-4-((4-Amino-6-fluoro-5-(3-(2-fluoroethyl)-2-methyl-3H-imidazo[4,5-b]pyridin-5-yl)pyrrolo[2,1-f][1,2,4]triazin-2-yl)amino)-1-methylcyclohexan-1-ol 143.

Fluffy white solid (2.3 mg, 0.005 mmol, 9.5% yield). ¹H NMR (499 MHz, DMSO-d₆) δ ppm 1.13 (3H, s), 1.34-1.47 (4H, m), 1.51-1.63 (2H, m), 1.74-1.88 (2H, m), 2.61 (3H, s), 3.61 (1H, br dd, J=8.21, 4.38 Hz), 4.21 (1H, s), 4.56 (2H, dt, J=27.45, 4.65 Hz), 4.78 (2H, dt, J=47.15, 4.73 Hz), 5.86 (1H, d, J=8.21 Hz), 7.52 (1H, br s), 7.59 (1H, d, J=2.46 Hz), 7.69 (1H, d, J=8.21 Hz), 8.06 (1H, d, J=8.49 Hz), 10.02 (1H, br s); ESIMS found for C₂₂H₂₆F₂N₈O m/z 457.2 (M+1).

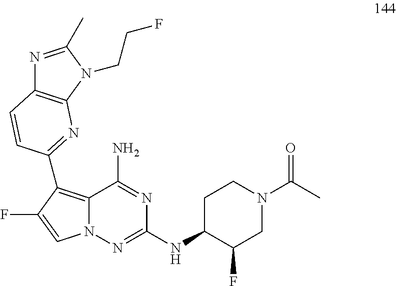 [SMILES: CC(=O)N1CC[C@H](Nc2nc(N)c3c(-c4ccc5nc(C)n(CCF)c5n4)c(F)cn3n2)[C@H](F)C1]

1-((3R,4S)-4-((4-Amino-6-fluoro-5-(3-(2-fluoroethyl)-2-methyl-3H-imidazo [4,5-b]pyridin-5-yl)pyrrolo[2,1-f][1,2,4]triazin-2-yl)amino)-3-fluoropiperidin-1-yl)ethan-1-one 144.

White solid (7 mg, 0.014 mmol, 93.1% yield). ¹H NMR (499 MHz, DMSO-d₆) δ ppm 1.58-1.70 (1H, m), 1.71-1.86 (1H, m), 1.99 (3H, s), 2.61 (3H, s), 2.64-2.93 (1H, m), 3.11-3.23 (1H, m), 3.83-3.98 (1H, m), 3.98-4.16 (1H, m), 4.37-4.49 (1H, m), 4.56 (2H, dt, J=27.40, 4.56 Hz), 4.78 (2H, dt, J=47.20, 4.73 Hz), 4.93 (1H, d, J=49.35 Hz), 6.09-6.13 (1H, m), 7.59 (1H, d, J=2.46 Hz), 7.65 (1H, br s), 7.70 (1H, d, J=8.21 Hz), 8.07 (1H, d, J=8.21 Hz), 10.11 (1H, br s); ESIMS found for C₂₂H₂₄F₃N₉O m/z 488.2 (M+1).

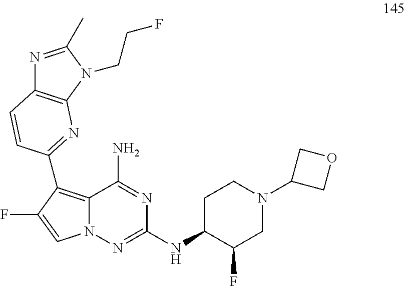 [SMILES: Cc1nc2ccc(-c3c(F)cn4nc(N[C@H]5CCN(C6COC6)C[C@H]5F)nc(N)c34)nc2n1CCF]

6-Fluoro-N2-((3R,4S)-3-fluoro-1-(oxetan-3-yl)piperidin-4-yl)-5-(3-(2-fluoroethyl)-2-methyl-3H-imidazo[4,5-b]pyridin-5-yl)pyrrolo[2,1-f][1,2,4]triazine-2,4-diamine 145.

Off-white solid ((3 mg, 0.006 mmol, 60.3% yield). ¹H NMR (499 MHz, DMSO-d₆) δ ppm 1.65-1.74 (1H, m), 1.85 (1H, qd, J=12.14, 3.56 Hz), 1.94-2.02 (1H, m), 2.10 (1H, dd, J=37.05, 12.59 Hz), 2.61 (3H, s), 2.69-2.78 (1H, m), 2.92-3.01 (1H, m), 3.48 (1H, quin, J=6.43 Hz), 3.70-3.87 (1H, m), 4.39 (1H, t, J=6.16 Hz), 4.45 (1H, t, J=6.16 Hz), 4.53 (2H, td, J=6.50, 2.87 Hz), 4.56 (2H, dt, J=27.40, 4.65 Hz), 4.78 (2H, dt, J=46.90, 4.79 Hz), 4.86 (1H, d, J=49.10 Hz), 5.99 (1H, d, J=8.21 Hz), 7.59 (1H, d, J=2.46 Hz), 7.64 (1H, br s), 7.70 (1H, d, J=8.21 Hz), 8.07 (1H, d, J=8.49 Hz), 10.10 (1H, br s); ESIMS found for C₂₃H₂₆F₃N₉O m/z 502.2 (M+1).

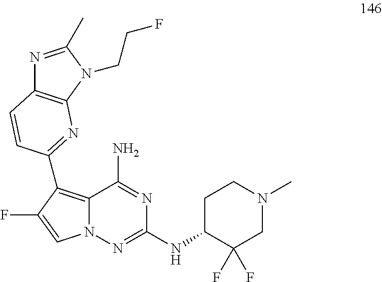 [SMILES: Cc1nc2ccc(-c3c(F)cn4nc(N[C@@H]5CCN(C)CC5(F)F)nc(N)c34)nc2n1CCF]

(R)—N²-(3,3-Difluoro-1-methylpiperidin-4-yl)-6-fluoro-5-(3-(2-fluoroethyl)-2-methyl-3H-imidazo[4,5-b]pyridin-5-yl)pyrrolo[2,1-f][1,2,4]triazine-2,4-diamine 146.

Fluffy white solid (3.8 mg, 0.008 mmol, 30.7% yield). ¹H NMR (499 MHz, DMSO-d₆) δ ppm 1.71-1.86 (2H, m), 2.06-2.17 (1H, m), 2.25 (3H, s), 2.31 (1H, ddd, J=27.15, 13.15, 1.65 Hz), 2.61 (3H, s), 2.77 (1H, br d, J=11.23 Hz), 2.98-3.10 (1H, m), 4.11-4.28 (1H, m), 4.56 (2H, dt, J=27.15, 4.65 Hz), 4.78 (2H, dt, J=47.20, 4.73 Hz), 6.19 (1H, br d, J=9.31 Hz), 7.60 (1H, d, J=2.46 Hz), 7.69 (1H, br s), 7.70 (1H, d, J=8.49 Hz), 8.07 (1H, d, J=8.49 Hz), 10.15 (1H, br s); ESIMS found for C₂₁H₂₃F₄N₉ m/z 478.2 (M+1).

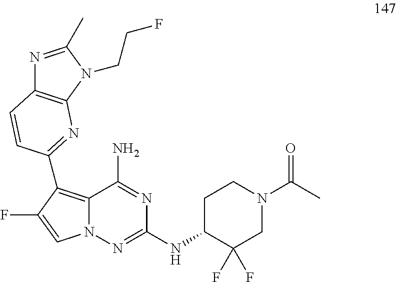 [SMILES: CC(=O)N1CC[C@@H](Nc2nc(N)c3c(-c4ccc5nc(C)n(CCF)c5n4)c(F)cn3n2)C(F)(F)C1]

(R)-1-(4-((4-Amino-6-fluoro-5-(3-(2-fluoroethyl)-2-methyl-3H-imidazo[4,5-b]pyridin-5-yl)pyrrolo[2,1-f][1,2,4]triazin-2-yl)amino)-3,3-difluoropiperidin-1-yl)ethan-1-one 147.

White solid (13 mg, 0.026 mmol, 64.1% yield). ¹H NMR (499 MHz, DMSO-d₆) δ ppm 1.53-1.79 (1H, m), 1.80-1.96 (1H, m), 2.06 (3H, s), 2.61 (3H, s), 2.96 (1H, br t, J=10.95 Hz), 3.60-3.89 (1H, m), 4.11-4.28 (1H, m), 4.40-4.51 (2H, m), 4.57 (2H, dt, J=27.40, 4.65 Hz), 4.78 (2H, dt, J=47.20, 4.65 Hz), 6.40 (1H, br d, J=9.31 Hz), 7.59 (1H, d, J=2.46 Hz), 7.69 (1H, br s), 7.71 (1H, d, J=8.21 Hz), 8.07 (1H, d, J=8.21 Hz), 10.16 (1H, br s); ESIMS found for C₂₂H₂₃F₄N₉O m/z 506.2 (M+1).

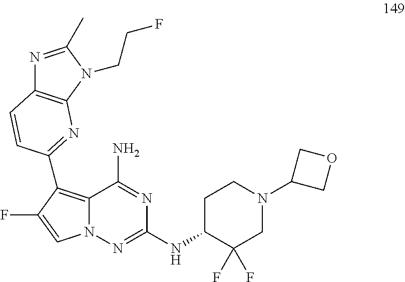 [SMILES: Cc1nc2ccc(-c3c(F)cn4nc(N[C@@H]5CCN(C6COC6)CC5(F)F)nc(N)c34)nc2n1CCF]

(R)—N²-(3,3-Difluoro-1-(oxetan-3-yl)piperidin-4-yl)-6-fluoro-5-(3-(2-fluoroethyl)-2-methyl-3H-imidazo[4,5-b]pyridin-5-yl)pyrrolo[2,1-f][1,2,4]triazine-2,4-diamine 149.

White solid (11 mg, 0.021 mmol, 52.8% yield). ¹H NMR (499 MHz, DMSO-d₆) δ ppm 1.70-1.81 (1H, m), 1.82-1.90 (1H, m), 2.04-2.16 (1H, m), 2.33 (1H, dd, J=26.35, 12.05 Hz), 2.61 (3H, s), 2.74 (1H, br d, J=10.95 Hz), 2.92-3.05 (1H, m), 3.59 (1H, quin, J=6.16 Hz), 4.19-4.34 (1H, m), 4.38-4.48 (2H, m), 4.54 (2H, td, J=6.50, 2.87 Hz), 4.56 (2H, dt, J=27.40, 4.65 Hz), 4.78 (2H, dt, J=47.15, 4.65 Hz), 6.27 (1H, br d, J=9.86 Hz), 7.60 (1H, d, J=2.19 Hz), 7.68 (1H, br s), 7.70 (1H, d, J=8.21 Hz), 8.07 (1H, d, J=8.21 Hz), 10.15 (1H, br s); ESIMS found for C₂₃H₂₅F₄N₉O m/z 520.2 (M+1).

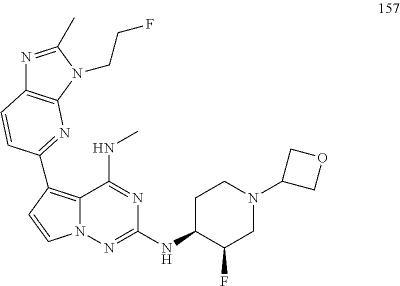 [SMILES: CNc1nc(N[C@H]2CCN(C3COC3)C[C@H]2F)nn2ccc(-c3ccc4nc(C)n(CCF)c4n3)c12]

N²-((3R,4S)-3-Fluoro-1-(oxetan-3-yl)piperidin-4-yl)-5-(3-(2-fluoroethyl)-2-methyl-3H-imidazo[4,5-b]pyridin-5-yl)-N⁴-methylpyrrolo[2,1-f][1,2,4]triazine-2,4-diamine 157.

Fluffy white solid (8 mg, 0.016 mmol, 19.1% yield). ¹H NMR (499 MHz, DMSO-d₆) δ ppm 1.67-1.75 (1H, m), 1.81-1.92 (1H, m), 1.96-2.04 (1H, m), 2.13 (1H, dd, J=37.60, 12.59 Hz), 2.61 (3H, s), 2.74 (1H, br d, J=10.40 Hz), 2.92-3.02 (1H, m), 3.11 (3H, d, J=4.65 Hz), 3.48 (1H, quin, J=6.37 Hz), 3.73-3.90 (1H, m), 4.40 (1H, t, J=6.02 Hz), 4.45 (1H, t, J=6.16 Hz), 4.54 (2H, td, J=6.30, 3.01 Hz), 4.60 (2H, dt, J=26.60, 4.65 Hz), 4.89 (1H, d, J=49.50 Hz), 4.89 (2H, dt, J=47.20, 4.65 Hz), 5.92 (1H, br d, J=7.94 Hz), 7.09 (1H, d, J=2.74 Hz), 7.35 (1H, d, J=2.74 Hz), 7.81 (1H, d, J=8.49 Hz), 7.98 (1H, d, J=8.49 Hz), 11.11 (1H, q, J=4.15 Hz); ESIMS found for C₂₄H₂₉F₂N₉O m/z 498.2 (M+1).

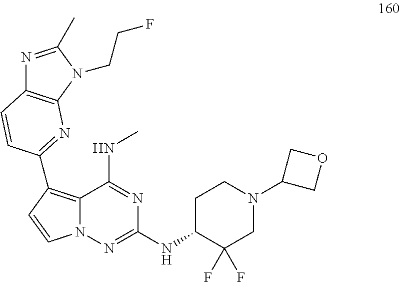 [SMILES: CNc1nc(N[C@@H]2CCN(C3COC3)CC2(F)F)nn2ccc(-c3ccc4nc(C)n(CCF)c4n3)c12]

(R)—N²-(3,3-Difluoro-1-(oxetan-3-yl)piperidin-4-yl)-5-(3-(2-fluoroethyl)-2-methyl-3H-imidazo[4,5-b]pyridin-5-yl)-N⁴-methylpyrrolo[2,1-f][1,2,4]triazine-2,4-diamine 160.

Off-white solid (9 mg, 0.018 mmol, 32.1% yield). ¹H NMR (499 MHz, DMSO-d₆) δ ppm 1.71-1.84 (1H, m), 1.84-1.93 (1H, m), 2.14 (1H, br t, J=10.54 Hz), 2.38 (1H, dd, J=26.85, 12.05 Hz), 2.61 (3H, s), 2.74 (1H, br d, J=10.95 Hz), 2.95-3.05 (1H, m), 3.13 (3H, d, J=4.65 Hz), 3.60 (1H, quin, J=6.30 Hz), 4.21-4.37 (1H, m), 4.44 (2H, dt, J=14.44, 6.19 Hz), 4.55 (2H, td, J=6.64, 3.42 Hz), 4.61 (2H, dt, J=26.90, 4.93 Hz), 4.90 (2H, dt, J=46.90, 4.93 Hz), 6.17 (1H, d, J=9.58 Hz), 7.10 (1H, d, J=3.01 Hz), 7.36 (1H, d, J=2.74 Hz), 7.82 (1H, d, J=8.76 Hz), 7.99 (1H, d, J=8.49 Hz), 11.14 (1H, q, J=4.95 Hz); ESIMS found for C₂₄H₂₈F₃N₉O m/z 516.2 (M+1).

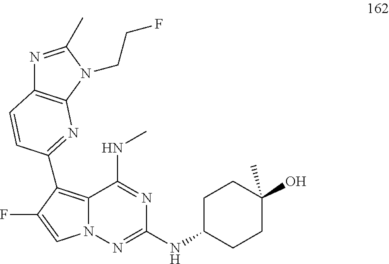 [SMILES: CNc1nc(N[C@H]2CC[C@](C)(O)CC2)nn2cc(F)c(-c3ccc4nc(C)n(CCF)c4n3)c12]

(1r,4r)-4-((6-Fluoro-5-(3-(2-fluoroethyl)-2-methyl-3H-imidazo[4,5-b]pyridin-5-yl)-4-(methylamino)pyrrolo[2,1-f][1,2,4]triazin-2-yl)amino)-1-methylcyclohexan-1-ol 162.

Fluffy white solid (4.8 mg, 0.010 mmol, 97.0% yield). ¹H NMR (499 MHz, DMSO-d₆) δ ppm 1.14 (3H, s), 1.36-1.47 (4H, m), 1.54-1.61 (2H, m), 1.79-1.88 (2H, m), 2.63 (3H, s), 3.06 (3H, d, J=4.65 Hz), 3.61 (1H, br d, J=2.74 Hz), 4.21 (1H, s), 4.61 (2H, dt, J=26.60, 4.79 Hz), 4.88 (2H, dt, J=46.90, 4.79 Hz), 5.97 (1H, d, J=7.94 Hz), 7.57 (1H, d, J=2.19 Hz), 7.70 (1H, d, J=8.49 Hz), 8.07 (1H, d, J=8.49 Hz), 10.31 (1H, q, J=4.56 Hz); ESIMS found for C₂₃H₂₈F₂N₈O m/z 471.2 (M+1).

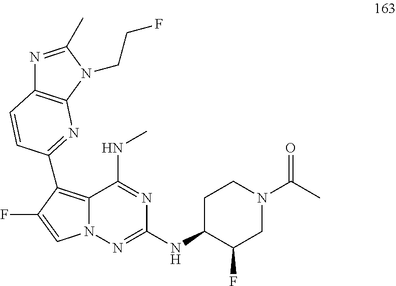 [SMILES: CNc1nc(N[C@H]2CCN(C(C)=O)C[C@H]2F)nn2cc(F)c(-c3ccc4nc(C)n(CCF)c4n3)c12]

1-((3R,4S)-3-Fluoro-4-((6-fluoro-5-(3-(2-fluoroethyl)-2-methyl-3H-imidazo [4,5-b]pyridin-5-yl)-4-(methylamino)pyrrolo[2,1-f][1,2,4]triazin-2-yl)amino)piperidin-1-yl)ethan-1-one 163.

White solid (9.4 mg, 0.019 mmol, 86.1% yield). ¹H NMR (499 MHz, DMSO-d₆) δ ppm 1.61-1.71 (1H, m), 1.72-1.88 (1H, m), 1.99 (3H, s), 2.63 (3H, s), 2.66-2.94 (1H, m), 3.08 (3H, d, J=4.65 Hz), 3.13-3.25 (1H, m), 3.84-3.97 (1H, m), 3.98-4.16 (1H, m), 4.43 (1H, br dd, J=12.32, 2.46 Hz), 4.61 (2H, dt, J=26.60, 4.79 Hz), 4.89 (2H, dt, J=46.65, 4.79 Hz), 4.95 (1H, d, J=49.35 Hz), 6.22 (1H, dd, J=7.94, 2.46 Hz), 7.56 (1H, d, J=2.46 Hz), 7.71 (1H, d, J=8.21 Hz), 8.08 (1H, d, J=8.49 Hz), 10.38-10.45 (1H, m); ESIMS found for C₂₃H₂₆F₃N₉O m/z 502.4 (M+1).

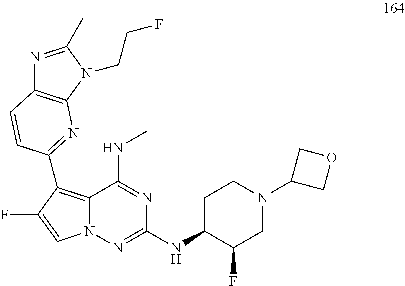 [SMILES: CNc1nc(N[C@H]2CCN(C3COC3)C[C@H]2F)nn2cc(F)c(-c3ccc4nc(C)n(CCF)c4n3)c12]

6-Fluoro-N²-((3R,4S)-3-fluoro-1-(oxetan-3-yl)piperidin-4-yl)-5-(3-(2-fluoroethyl)-2-methyl-3H-imidazo[4,5-b]pyridin-5-yl)-N⁴-methylpyrrolo[2,1-f][1,2,4]triazine-2,4-diamine 164.

Fluffy white solid (11 mg, 0.021 mmol, 59.4% yield). ¹H NMR (499 MHz, DMSO-d₆) δ ppm 1.65-1.76 (1H, m), 1.87 (1H, qd, J=12.18, 3.70 Hz), 1.95-2.05 (1H, m), 2.12 (1H, dd, J=36.75, 12.56 Hz), 2.63 (3H, s), 2.74 (1H, br d, J=11.22 Hz), 2.97 (1H, br t, J=11.64 Hz), 3.07 (3H, d, J=4.65 Hz), 3.48 (1H, quin, J=6.23 Hz), 3.69-3.89 (1H, m), 4.39 (1H, t, J=6.16 Hz), 4.45 (1H, t, J=6.16 Hz), 4.53 (2H, td, J=6.57, 3.01 Hz), 4.61 (2H, dt, J=26.60, 4.79 Hz), 4.89 (2H, dt, J=46.90, 4.65 Hz), 4.89 (1H, d, J=49.50 Hz), 6.10 (1H, d, J=8.21 Hz), 7.56 (1H, d, J=2.19 Hz), 7.71 (1H, d, J=8.21 Hz), 8.08 (1H, d, J=8.49 Hz), 10.40 (1H, q, J=4.56 Hz); ESIMS found for C₂₄H₂₈F₃N₉O m/z 516.2 (M+1).

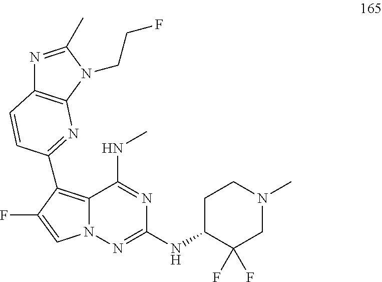 [SMILES: CNc1nc(N[C@@H]2CCN(C)CC2(F)F)nn2cc(F)c(-c3ccc4nc(C)n(CCF)c4n3)c12]

(R)—N²-(3,3-Difluoro-1-methylpiperidin-4-yl)-6-fluoro-5-(3-(2-fluoroethyl)-2-methyl-3H-imidazo[4,5-b]pyridin-5-yl)-N⁴-methylpyrrolo[2,1-f][1,2,4]triazine-2,4-diamine 165.

Fluffy white solid (11.7 mg, 0.024 mmol, 56.8% yield). ¹H NMR (499 MHz, DMSO-d₆) δ ppm 1.71-1.89 (2H, m), 2.09-2.19 (1H, m), 2.25 (3H, s), 2.35 (1H, dd, J=27.15, 12.32 Hz), 2.63 (3H, s), 2.77 (1H, br d, J=11.23 Hz), 3.00-3.07 (1H, m), 3.09 (3H, d, J=4.93 Hz), 4.09-4.28 (1H, m), 4.61 (2H, dt, J=26.60, 4.79 Hz), 4.89 (2H, dt, J=46.90, 4.93 Hz), 6.26 (1H, d, J=9.31 Hz), 7.58 (1H, d, J=2.19 Hz), 7.71 (1H, d, J=8.49 Hz), 8.08 (1H, d, J=8.49 Hz), 10.42 (1H, q, J=4.38 Hz); ESIMS found for C₂₂H₂₅F₄N₉ m/z 492.2 (M+1).

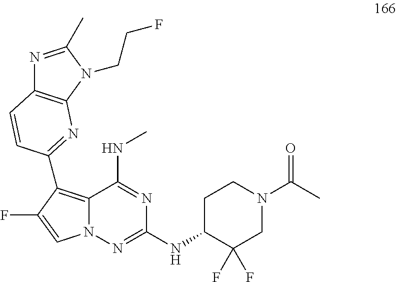 [SMILES: CNc1nc(N[C@@H]2CCN(C(C)=O)CC2(F)F)nn2cc(F)c(-c3ccc4nc(C)n(CCF)c4n3)c12]

(R)-1-(3,3-Difluoro-4-((6-fluoro-5-(3-(2-fluoroethyl)-2-methyl-3H-imidazo [4,5-b]pyridin-5-yl)-4-(methylamino)pyrrolo[2,1-f][1,2,4]triazin-2-yl)amino)piperidin-1-yl)ethan-1-one 166.

White solid (4.2 mg, 0.008 mmol, 24.1% yield). ¹H NMR (499 MHz, DMSO-d₆) δ ppm 1.56-1.79 (1H, m), 1.81-1.99 (1H, m), 2.06 (3H, s), 2.64 (3H, s), 2.91-3.04 (1H, m), 3.10 (3H, d, J=4.93 Hz), 3.60-3.91 (1H, m), 4.07-4.29 (1H, m), 4.39-4.56 (2H, m), 4.61 (2H, dt, J=26.90, 4.65 Hz), 4.89 (2H, dt, J=46.60, 4.79 Hz), 6.46-6.50 (1H, m), 7.57 (1H, s), 7.71 (1H, d, J=8.21 Hz), 8.08 (1H, d, J=8.49 Hz), 10.44 (1H, q, J=4.47 Hz); ESIMS found for C₂₃H₂₅F₄N₉O m/z 520.2 (M+1).

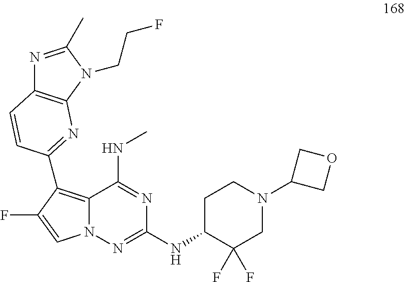 [SMILES: CNc1nc(N[C@@H]2CCN(C3COC3)CC2(F)F)nn2cc(F)c(-c3ccc4nc(C)n(CCF)c4n3)c12]

(R)—N²-(3,3-Difluoro-1-(oxetan-3-yl)piperidin-4-yl)-6-fluoro-5-(3-(2-fluoroethyl)-2-methyl-3H-imidazo[4,5-b]pyridin-5-yl)-N⁴-methylpyrrolo[2,1-f][1,2,4]triazine-2,4-diamine 168.

Fluffy white solid (12.3 mg, 0.023 mmol, 55.0% yield). ¹H NMR (499 MHz, DMSO-d₆) δ ppm 1.70-1.82 (1H, m), 1.83-1.92 (1H, m), 2.08-2.19 (1H, m), 2.37 (1H, dd, J=26.60, 12.32 Hz), 2.63 (3H, s), 2.74 (1H, br d, J=11.77 Hz), 2.93-3.05 (1H, m), 3.09 (3H, d, J=4.65 Hz), 3.59 (1H, quin, J=6.37 Hz), 4.17-4.34 (1H, m), 4.43 (2H, dt, J=14.44, 6.19 Hz), 4.54 (2H, td, J=6.71, 3.56 Hz), 4.61 (2H, dt, J=26.85, 4.79 Hz), 4.89 (2H, dt, J=46.90, 4.79 Hz), 6.34 (1H, d, J=9.58 Hz), 7.57 (1H, d, J=2.19 Hz), 7.71 (1H, d, J=8.21 Hz), 8.08 (1H, d, J=8.49 Hz), 10.42 (1H, q, J=4.56 Hz); ESIMS found for C₂₄H₂₇F₄N₉O m/z 534.2 (M+1).

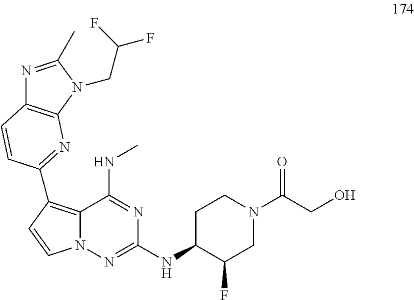 [SMILES: CNc1nc(N[C@H]2CCN(C(=O)CO)C[C@H]2F)nn2ccc(-c3ccc4nc(C)n(CC(F)F)c4n3)c12]

1-((3R,4S)-4-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)-3-fluoropiperidin-1-yl)-2-hydroxyethan-1-one 174.

Fluffy pale-yellow solid (23 mg, 0.044 mmol, 72.1% yield). ¹H NMR (499 MHz, DMSO-d₆) δ ppm 1.64-1.88 (2H, m), 2.62 (3H, s), 2.72-3.04 (1H, m), 3.13 (3H, d, J=4.65 Hz), 3.76 (1H, br d, J=13.96 Hz), 3.94-4.19 (4H, m), 4.55-4.60 (1H, m), 4.66 (1H, br t, J=12.59 Hz), 4.78 (2H, td, J=15.88, 2.46 Hz), 4.96 (1H, d, J=49.10 Hz), 6.12 (1H, d, J=3.56 Hz), 6.64 (1H, tt, J=54.30, 3.01 Hz), 7.11 (1H, d, J=3.01 Hz), 7.37 (1H, d, J=2.74 Hz), 7.85 (1H, d, J=8.76 Hz), 8.01 (1H, d, J=8.49 Hz), 10.99 (1H, q, J=4.47 Hz); ESIMS found for C₂₃H₂₆F₃N₉O₂ m/z 518.2 (M+1).

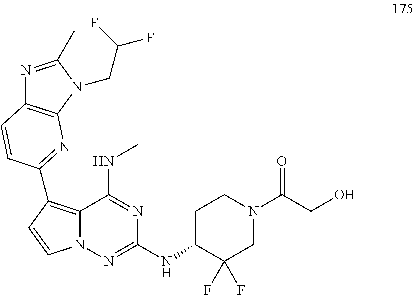 [SMILES: CNc1nc(N[C@@H]2CCN(C(=O)CO)CC2(F)F)nn2ccc(-c3ccc4nc(C)n(CC(F)F)c4n3)c12]

(R)-1-(4-((5-(3-(2,2-Difluoroethyl)-2-methyl-3H-imidazo[4,5-b]pyridin-5-yl)-4-(methylamino)pyrrolo[2,1-f][1,2,4]triazin-2-yl)amino)-3,3-difluoropiperidin-1-yl)-2-hydroxyethan-1-one 175.

Fluffy pale-yellow solid (34 mg, 0.064 mmol, 82.1% yield). ¹H NMR (499 MHz, DMSO-d₆) δ ppm 1.61-1.82 (1H, m), 1.84-1.98 (1H, m), 2.63 (3H, s), 3.00-3.10 (1H, m), 3.15 (3H, d, J=4.65 Hz), 3.60-3.83 (1H, m), 4.03-4.27 (3H, m), 4.39-4.62 (2H, m), 4.78 (2H, td, J=15.95, 2.79 Hz), 4.79-4.84 (1H, m), 6.36 (1H, br d, J=9.58 Hz), 6.64 (1H, tt, J=54.30, 3.01 Hz), 7.12 (1H, d, J=3.01 Hz), 7.37 (1H, d, J=3.01 Hz), 7.86 (1H, d, J=8.49 Hz), 8.01 (1H, d, J=8.49 Hz), 11.02 (1H, q, J=4.56 Hz); ESIMS found for C₂₃H₂₅F₄N₉O₂ m/z 536.2 (M+1).

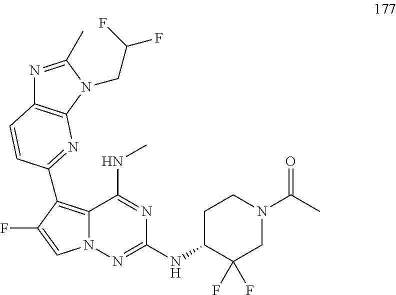 [SMILES: CNc1nc(N[C@@H]2CCN(C(C)=O)CC2(F)F)nn2cc(F)c(-c3ccc4nc(C)n(CC(F)F)c4n3)c12]

(R)-1-(4-((5-(3-(2,2-Difluoroethyl)-2-methyl-3H-imidazo[4,5-b]pyridin-5-yl)-6-fluoro-4-(methylamino)pyrrolo[2,1-f][1,2,4]triazin-2-yl)amino)-3,3-difluoropiperidin-1-yl)ethan-1-one 177.

Fluffy white solid (10 mg, 0.019 mmol, 98.0% yield). ¹H NMR (499 MHz, DMSO-d₆) δ ppm 1.56-1.80 (1H, m), 1.81-1.96 (1H, m), 2.06 (3H, s), 2.64 (3H, s), 2.92-3.03 (1H, m), 3.11 (3H, d, J=4.65 Hz), 3.63-3.90 (1H, m), 4.09-4.26 (1H, m), 4.42-4.58 (2H, m), 4.78 (2H, td, J=15.95, 2.79 Hz), 6.49 (1H, d, J=9.58 Hz), 6.63 (2H, tt, J=54.30, 3.15 Hz), 7.58 (1H, d, J=2.19 Hz), 7.74 (1H, d, J=8.21 Hz), 8.11 (1H, d, J=8.49 Hz), 10.26 (1H, q, J=4.56 Hz); ESIMS found for C₂₃H₂₄F₅N₉O m/z 538.2 (M+1).

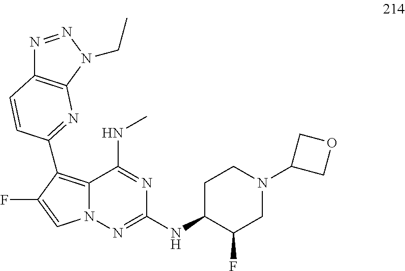 [SMILES: CCn1nnc2ccc(-c3c(F)cn4nc(N[C@H]5CCN(C6COC6)C[C@H]5F)nc(NC)c34)nc21]

5-(3-Ethyl-3H-[1,2,3]triazolo[4,5-b]pyridin-5-yl)-6-fluoro-N²-((3R,4S)-3-fluoro-1-(oxetan-3-yl)piperidin-4-yl)-N⁴-methylpyrrolo[2,1-f][1,2,4]triazine-2,4-diamine 214.

Light yellow solid (21 mg, 0.043 mmol, 60.1% yield). ¹H NMR (499 MHz, DMSO-d₆) δ ppm 1.62 (3H, t, J=7.39 Hz), 1.67-1.75 (1H, m), 1.89 (1H, qd, J=12.14, 3.83 Hz), 1.95-2.04 (1H, m), 2.13 (1H, dd, J=37.05, 12.59 Hz), 2.70-2.80 (1H, m), 2.93-3.04 (1H, m), 3.11 (3H, d, J=4.65 Hz), 3.49 (1H, quin, J=6.37 Hz), 3.70-3.88 (1H, m), 4.40 (1H, t, J=6.16 Hz), 4.45 (1H, t, J=6.16 Hz), 4.54 (2H, td, J=6.57, 3.01 Hz), 4.75 (2H, q, J=7.30 Hz), 4.89 (1H, d, J=49.35 Hz), 6.28 (1H, d, J=8.21 Hz), 7.63 (1H, d, J=2.19 Hz), 7.91 (1H, d, J=9.03 Hz), 8.61 (1H, d, J=9.04 Hz), 10.54 (1H, q, J=4.65 Hz); ESIMS found for C₂₂H₂₆F₂N₁₀O m/z 485.2 (M+1).

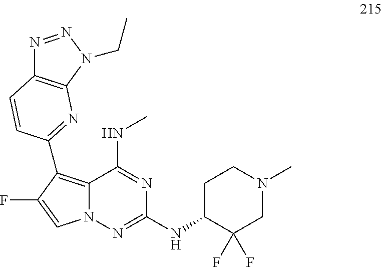 [SMILES: CCn1nnc2ccc(-c3c(F)cn4nc(N[C@@H]5CCN(C)CC5(F)F)nc(NC)c34)nc21]

(R)—N²-(3,3-Difluoro-1-methylpiperidin-4-yl)-5-(3-ethyl-3H-[1,2,3]triazolo [4,5-b]pyridin-5-yl)-6-fluoro-N⁴-methylpyrrolo[2,1-f][1,2,4]triazine-2,4-diamine 215.

Light yellow solid (17 mg, 0.037 mmol, 91.6% yield). ¹H NMR (499 MHz, DMSO-d₆) δ ppm 1.62 (3H, t, J=7.39 Hz), 1.73-1.88 (2H, m), 2.15 (1H, td, J=11.23, 2.46 Hz), 2.25 (3H, s), 2.35 (1H, ddd, J=27.15, 12.35, 1.90 Hz), 2.73-2.85 (1H, m), 2.98-3.09 (1H, m), 3.12 (3H, d, J=4.65 Hz), 4.12-4.30 (1H, m), 4.76 (2H, q, J=7.39 Hz), 6.45 (1H, d, J=9.58 Hz), 7.65 (1H, d, J=2.19 Hz), 7.92 (1H, d, J=9.03 Hz), 8.62 (1H, d, J=8.76 Hz), 10.55 (1H, q, J=4.65 Hz); ESIMS found for C₂₀H₂₃F₃N₁₀ m/z 461.2 (M+1).

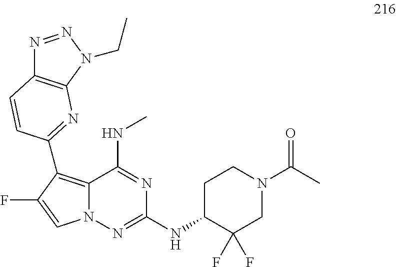 [SMILES: CCn1nnc2ccc(-c3c(F)cn4nc(N[C@@H]5CCN(C(C)=O)CC5(F)F)nc(NC)c34)nc21]

(R)-1-(4-((5-(3-Ethyl-3H-[1,2,3]triazolo[4,5-b]pyridin-5-yl)-6-fluoro-4-(methylamino)pyrrolo[2,1-f][1,2,4]triazin-2-yl)amino)-3,3-difluoropiperidin-1-yl)ethan-1-one 216.

Light yellow solid (19 mg, 0.039 mmol, 96.5% yield). ¹H NMR (499 MHz, DMSO-d₆) δ ppm 1.62 (3H, t, J=7.26 Hz), 1.71-1.81 (1H, m), 1.82-1.96 (1H, m), 2.07 (3H, s), 2.91-3.04 (1H, m), 3.13 (3H, d, J=4.65 Hz), 3.62-3.91 (1H, m), 4.09-4.30 (1H, m), 4.41-4.58 (2H, m), 4.76 (2H, q, J=7.39 Hz), 6.64 (1H, br d, J=9.58 Hz), 7.62-7.65 (1H, m), 7.92 (1H, d, J=9.03 Hz), 8.62 (1H, d, J=9.03 Hz), 10.58 (1H, q, J=4.38 Hz); ESIMS found for C₂₁H₂₃F₃N₁₀O m/z 489.2 (M+1).

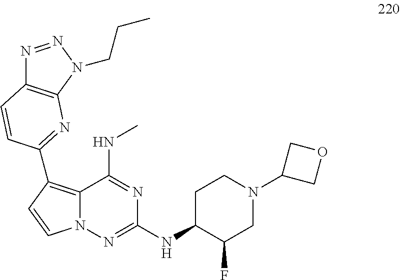 [SMILES: CCCn1nnc2ccc(-c3ccn4nc(N[C@H]5CCN(C6COC6)C[C@H]5F)nc(NC)c34)nc21]

N²-((3R,4S)-3-Fluoro-1-(oxetan-3-yl)piperidin-4-yl)-5-(3-(2-fluoroethyl)-3H-[1,2,3]triazolo[4,5-b]pyridin-5-yl)pyrrolo[2,1-f][1,2,4]triazine-2,4-diamine 220.

Yellow solid (8 mg, 0.017 mmol, 19.3% yield). ¹H NMR (499 MHz, DMSO-d₆) δ ppm 1.70 (1H, br d, J=10.40 Hz), 1.81-1.91 (1H, m), 1.93-2.03 (1H, m), 2.12 (1H, dd, J=37.05, 12.59 Hz), 2.69-2.81 (1H, m), 2.90-3.04 (1H, m), 3.48 (1H, quin, J=5.89 Hz), 3.72-3.90 (1H, m), 4.39 (1H, br t, J=6.02 Hz), 4.45 (1H, br t, J=5.89 Hz), 4.50-4.58 (2H, m), 4.88 (1H, d, J=49.65 Hz), 4.93 (2H, dt, J=36.20, 4.65 Hz), 5.01 (2H, dt, J=16.20, 4.65 Hz), 6.07 (1H, br d, J=7.94 Hz), 7.34 (1H, d, J=3.01 Hz), 7.45 (1H, d, J=3.01 Hz), 7.70 (1H, br s), 8.08 (1H, d, J=9.03 Hz), 8.53 (1H, d, J=9.03 Hz), 10.58 (1H, br s); ESIMS found for C₂₁H₂₄F₂N₁₀O m/z 471.2 (M+1).

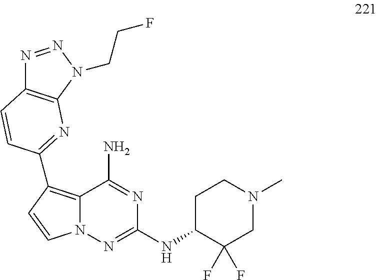 [SMILES: CN1CC[C@@H](Nc2nc(N)c3c(-c4ccc5nnn(CCF)c5n4)ccn3n2)C(F)(F)C1]

(R)—N²-(3,3-Difluoro-1-methylpiperidin-4-yl)-5-(3-(2-fluoroethyl)-3H-[1,2,3]triazolo[4,5-b]pyridin-5-yl)pyrrolo[2,1-f][1,2,4]triazine-2,4-diamine 221.

Light yellow solid (5 mg, 0.011 mmol, 17.7% yield). ¹H NMR (499 MHz, DMSO-d₆) δ ppm 1.70-1.88 (2H, m), 2.07-2.18 (1H, m), 2.25 (3H, s), 2.33 (1H, dd, J=27.70, 11.77 Hz), 2.77 (1H, br d, J=11.77 Hz), 2.98-3.11 (1H, m), 4.12-4.31 (1H, m), 4.93 (2H, dt, J=36.50, 4.65 Hz), 5.01 (2H, dt, J=16.75, 4.30 Hz), 6.25 (1H, d, J=9.58 Hz), 7.35 (1H, d, J=3.01 Hz), 7.47 (1H, d, J=3.01 Hz), 7.75 (1H, br s), 8.08 (1H, d, J=9.04 Hz), 8.53 (1H, d, J=8.76 Hz), 10.61 (1H, br s); ESIMS found for C₁₉H₂₁F₃N₁₀ m/z 447.2 (M+1).

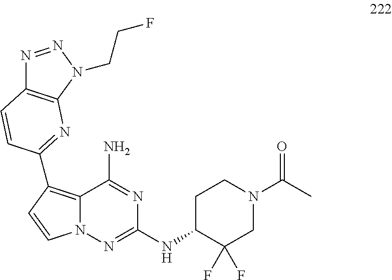 [SMILES: CC(=O)N1CC[C@@H](Nc2nc(N)c3c(-c4ccc5nnn(CCF)c5n4)ccn3n2)C(F)(F)C1]

(R)-1-(4-((4-Amino-5-(3-(2-fluoroethyl)-3H-[1,2,3]triazolo[4,5-b]pyridin-5-yl)pyrrolo[2,1-f][1,2,4]triazin-2-yl)amino)-3,3-difluoropiperidin-1-yl)ethan-1-one 222.

Light yellow solid (8 mg, 0.017 mmol, 24.2% yield). ¹H NMR (499 MHz, DMSO-d₆) δ ppm 1.52-1.80 (1H, m), 1.82-1.98 (1H, m), 2.01-2.09 (3H, m), 2.92-3.06 (1H, m), 3.61-3.91 (1H, m), 4.08-4.27 (1H, m), 4.40-4.59 (2H, m), 4.94 (2H, dt, J=37.30, 4.90 Hz), 4.99-5.05 (2H, m), 6.48 (1H, br d, J=3.01 Hz), 7.37 (1H, d, J=3.29 Hz), 7.46 (1H, dd, J=2.87, 1.51 Hz), 7.75 (1H, br s), 8.09 (1H, d, J=9.03 Hz), 8.54 (1H, d, J=8.76 Hz), 10.63 (1H, br s); ESIMS found for C₂₀H₂₁F₃N₁₀O m/z 475.2 (M+1).

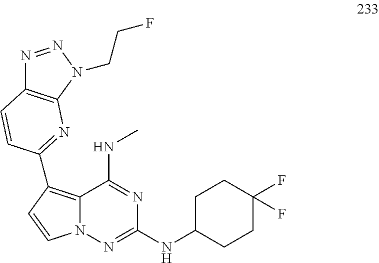 [SMILES: CNc1nc(NC2CCC(F)(F)CC2)nn2ccc(-c3ccc4nnn(CCF)c4n3)c12]

N²-(4,4-Difluorocyclohexyl)-5-(3-(2-fluoroethyl)-3H-[1,2,3]triazolo[4,5-b]pyridin-5-yl)-N⁴-methylpyrrolo[2,1-f][1,2,4]triazine-2,4-diamine 233.

Fluffy yellow solid (8 mg, 0.018 mmol, 43.6% yield). ¹H NMR (499 MHz, DMSO-d₆) δ ppm 1.55-1.70 (2H, m), 1.80-2.01 (4H, m), 2.02-2.17 (2H, m), 3.13 (3H, d, J=4.65 Hz), 3.72-3.86 (1H, m), 4.97-5.05 (2H, m), 5.08 (2H, s), 6.31 (1H, d, J=7.67 Hz), 7.31 (1H, d, J=3.01 Hz), 7.43 (1H, d, J=3.01 Hz), 8.07 (1H, d, J=9.03 Hz), 8.53 (1H, d, J=8.76 Hz), 10.95 (1H, q, J=4.56 Hz); ESIMS found for C₂₀H₂₂F₃N₉ m/z 446.2 (M+1).

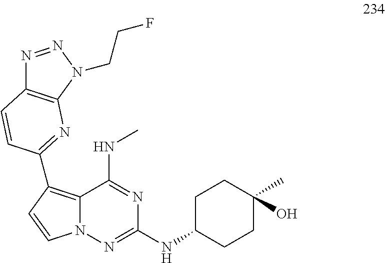 [SMILES: CNc1nc(N[C@H]2CC[C@](C)(O)CC2)nn2ccc(-c3ccc4nnn(CCF)c4n3)c12]

(1r,4r)-4-((5-(3-(2-Fluoroethyl)-3H-[1,2,3]triazolo[4,5-b]pyridin-5-yl)-4-(methylamino)pyrrolo[2,1-f][1,2,4]triazin-2-yl)amino)-1-methylcyclohexan-1-ol 234.

Yellow solid (16 mg, 0.036 mmol, 42.0% yield). ¹H NMR (499 MHz, DMSO-d₆) δ ppm 1.14 (3H, s), 1.36-1.50 (4H, m), 1.54-1.65 (2H, m), 1.79-1.91 (2H, m), 3.12 (3H, d, J=4.38 Hz), 3.64 (1H, br s), 4.21 (1H, s), 5.00 (2H, dt, J=20.30, 4.40 Hz), 5.08 (2H, s), 6.02 (1H, d, J=8.21 Hz), 7.29 (1H, d, J=3.29 Hz), 7.43 (1H, d, J=2.74 Hz), 8.07 (1H, d, J=9.31 Hz), 8.52 (1H, d, J=9.31 Hz), 10.92 (1H, q, J=4.93 Hz); ESIMS found for C₂₁H₂₆FN₉O m/z 440.2 (M+1).

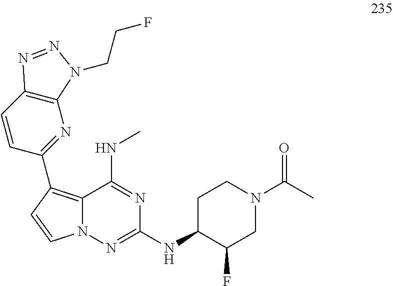 [SMILES: CNc1nc(N[C@H]2CCN(C(C)=O)C[C@H]2F)nn2ccc(-c3ccc4nnn(CCF)c4n3)c12]

1-((3R,4S)-3-Fluoro-4-((5-(3-(2-fluoroethyl)-3H-[1,2,3]triazolo[4,5-b]pyridin-5-yl)-4-(methylamino)pyrrolo[2,1-f][1,2,4]triazin-2-yl)amino)piperidin-1-yl)ethan-1-one 235.

Fluffy yellow solid (9 mg, 0.019 mmol, 50.1% yield). ¹H NMR (499 MHz, DMSO-d₆) δ ppm 1.59-1.89 (2H, m), 1.96-2.07 (3H, m), 2.59-2.78 (1H, m), 2.79-2.95 (1H, m), 3.13 (3H, d, J=4.65 Hz), 3.83-4.00 (1H, m), 4.01-4.19 (1H, m), 4.40-4.75 (1H, m), 4.89-5.02 (1H, m), 4.97-5.06 (2H, m), 5.08 (2H, s), 6.27 (1H, d, J=7.94 Hz), 7.33 (1H, d, J=3.29 Hz), 7.44 (1H, d, J=3.01 Hz), 8.09 (1H, d, J=9.31 Hz), 8.54 (1H, d, J=9.03 Hz), 11.01 (1H, q, J=4.95 Hz); ESIMS found for C₂₁H₂₄F₂N₁₀O m/z 471.2 (M+1).

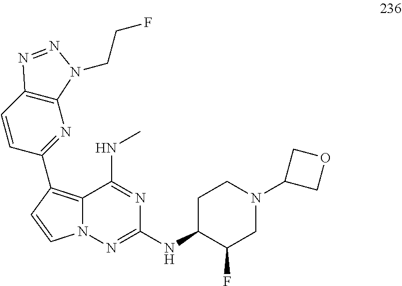 [SMILES: CNc1nc(N[C@H]2CCN(C3COC3)C[C@H]2F)nn2ccc(-c3ccc4nnn(CCF)c4n3)c12]

N²-((3R,4S)-3-Fluoro-1-(oxetan-3-yl)piperidin-4-yl)-5-(3-(2-fluoroethyl)-3H-[1,2,3]triazolo[4,5-b]pyridin-5-yl)-N⁴-methylpyrrolo[2,1-f][1,2,4]triazine-2,4-diamine 236.

Fluffy yellow solid (14 mg, 0.029 mmol, 78.9% yield). ¹H NMR (499 MHz, DMSO-d₆) δ ppm 1.67-1.75 (1H, m), 1.88 (1H, qd, J=12.05, 3.83 Hz), 1.96-2.04 (1H, m), 2.14 (1H, dd, J=37.00, 12.87 Hz), 2.75 (1H, br d, J=10.95 Hz), 2.92-3.03 (1H, m), 3.13 (3H, d, J=4.65 Hz), 3.49 (1H, quin, J=6.37 Hz), 3.74-3.91 (1H, m), 4.40 (1H, t, J=6.16 Hz), 4.46 (1H, t, J=6.16 Hz), 4.54 (2H, td, J=6.57, 3.29 Hz), 4.91 (1H, d, J=50.15 Hz), 4.97-5.05 (2H, m), 5.08 (2H, s), 6.15 (1H, d, J=7.94 Hz), 7.32 (1H, d, J=3.01 Hz), 7.43 (1H, d, J=3.01 Hz), 8.08 (1H, d, J=9.03 Hz), 8.53 (1H, d, J=9.04 Hz), 11.00 (1H, q, J=4.47 Hz); ESIMS found for C₂₂H₂₆F₂N₁₀O m/z 243.2 (M/2+1).

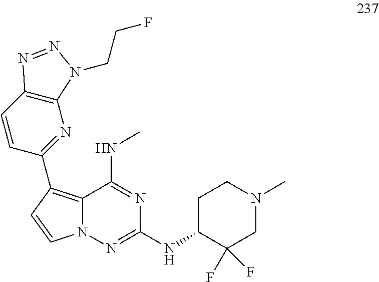 [SMILES: CNc1nc(N[C@@H]2CCN(C)CC2(F)F)nn2ccc(-c3ccc4nnn(CCF)c4n3)c12]

(R)—N²-(3,3-Difluoro-1-methylpiperidin-4-yl)-5-(3-(2-fluoroethyl)-3H-[1,2,3]triazolo[4,5-b]pyridin-5-yl)-N⁴-methylpyrrolo[2,1-f][1,2,4]triazine-2,4-diamine 237.

Fluffy yellow solid (14 mg, 0.030 mmol, 55.9% yield). ¹H NMR (499 MHz, DMSO-d₆) δ ppm 1.72-1.90 (2H, m), 2.10-2.21 (1H, m), 2.25 (3H, s), 2.36 (1H, dd, J=27.15, 12.39 Hz), 2.77 (1H, br d, J=11.50 Hz), 2.95-3.08 (1H, m), 3.14 (3H, d, J=4.65 Hz), 4.15-4.32 (1H, m), 4.96-5.05 (2H, m), 5.08 (2H, s), 6.31 (1H, d, J=9.31 Hz), 7.33 (1H, d, J=3.01 Hz), 7.44 (1H, d, J=3.01 Hz), 8.09 (1H, d, J=9.04 Hz), 8.54 (1H, d, J=8.76 Hz), 11.01 (1H, q, J=4.56 Hz); ESIMS found for C₂₀H₂₃F₃N₁₀ m/z 231.1 (M/2+1).

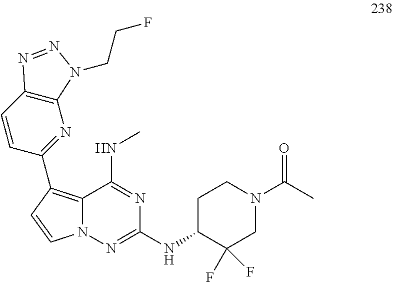 [SMILES: CNc1nc(N[C@@H]2CCN(C(C)=O)CC2(F)F)nn2ccc(-c3ccc4nnn(CCF)c4n3)c12]

(R)-1-(3,3-Difluoro-4-((5-(3-(2-fluoroethyl)-3H-[1,2,3]triazolo[4,5-b]pyridin-5-yl)-4-(methylamino)pyrrolo[2,1-f][1,2,4]triazin-2-yl)amino)piperidin-1-yl)ethan-1-one 238.

Fluffy yellow solid (7 mg, 0.014 mmol, 51.4% yield). ¹H NMR (499 MHz, DMSO-d₆) δ ppm 1.54-1.81 (1H, m), 1.83-1.99 (1H, m), 2.01-2.09 (3H, m), 2.89-3.06 (1H, m), 3.16 (3H, d, J=4.93 Hz), 3.59-3.90 (1H, m), 4.08-4.30 (1H, m), 4.40-4.61 (2H, m), 4.97-5.06 (2H, m), 5.09 (2H, s), 6.51 (1H, d, J=4.11 Hz), 7.34 (1H, d, J=3.29 Hz), 7.44 (1H, dd, J=2.87, 1.78 Hz), 8.09 (1H, d, J=9.03 Hz), 8.54 (1H, d, J=9.04 Hz), 10.99-11.09 (1H, m); ESIMS found for C₂₁H₂₃F₃N₁₀O m/z 489.25 (M+1).

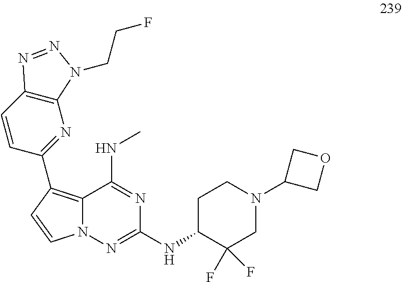 [SMILES: CNc1nc(N[C@@H]2CCN(C3COC3)CC2(F)F)nn2ccc(-c3ccc4nnn(CCF)c4n3)c12]

(R)—N²-(3,3-Difluoro-1-(oxetan-3-yl)piperidin-4-yl)-5-(3-(2-fluoroethyl)-3H-[1,2,3]triazolo[4,5-b]pyridin-5-yl)-N⁴-methylpyrrolo[2,1-f][1,2,4]triazine-2,4-diamine 239.

Fluffy yellow solid (13 mg, 0.026 mmol, 56.7% yield). ¹H NMR (499 MHz, DMSO-d₆) δ ppm 1.72-1.83 (1H, m), 1.84-1.92 (1H, m), 2.08-2.21 (1H, m), 2.32-2.44 (1H, m), 2.70-2.79 (1H, m), 2.93-3.06 (1H, m), 3.15 (3H, d, J=4.65 Hz), 3.60 (1H, quin, J=6.30 Hz), 4.20-4.37 (1H, m), 4.44 (2H, dt, J=14.58, 6.26 Hz), 4.55 (2H, td, J=6.64, 3.70 Hz), 4.96-5.06 (2H, m), 5.09 (2H, s), 6.39 (1H, d, J=9.58 Hz), 7.33 (1H, d, J=3.29 Hz), 7.44 (1H, d, J=3.01 Hz), 8.09 (1H, d, J=9.31 Hz), 8.54 (1H, d, J=9.04 Hz), 11.02 (1H, q, J=4.47 Hz); ESIMS found for C₂₂H₂₅F₃N₁₀O m/z 503.3 (M+1).

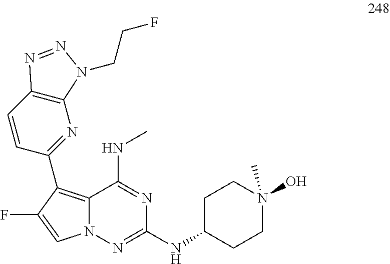

(1r,4r)-4-((6-Fluoro-5-(3-(2-fluoroethyl)-3H-[1,2,3]triazolo[4,5-b]pyridin-5-yl)-4-(methylamino)pyrrolo[2,1-f][1,2,4]triazin-2-yl)amino)-1-methylcyclohexan-1-ol 248.

Fluffy white solid (7.5 mg, 0.016 mmol, 67.8% yield). ¹H NMR (499 MHz, DMSO-d₆) δ ppm 1.14 (3H, s), 1.35-1.50 (4H, m), 1.52-1.64 (2H, m), 1.76-1.90 (2H, m), 3.08 (3H, d, J=4.93 Hz), 3.54-3.68 (1H, m), 4.21 (1H, s), 5.01 (2H, dt, J=20.55, 4.65 Hz), 5.08 (2H, s), 6.14 (1H, d, J=7.94 Hz), 7.65 (1H, d, J=2.19 Hz), 7.92 (1H, d, J=9.03 Hz), 8.64 (1H, d, J=9.03 Hz), 10.20 (1H, q, J=4.65 Hz); ESIMS found for C₂₁H₂₅F₂N₉O m/z 458.2 (M+1).

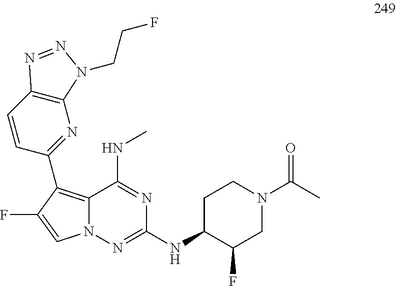 [SMILES: CNc1nc(N[C@H]2CCN(C(C)=O)C[C@H]2F)nn2cc(F)c(-c3ccc4nnn(CCF)c4n3)c12]

1-((3R,4S)-3-Fluoro-4-((6-fluoro-5-(3-(2-fluoroethyl)-3H-[1,2,3]triazolo[4,5-b]pyridin-5-yl)-4-(methylamino)pyrrolo[2,1-f][1,2,4]triazin-2-yl)amino)piperidin-1-yl)ethan-1-one 249.

Fluffy yellow solid (15 mg, 0.031 mmol, 82.5% yield). ¹H NMR (499 MHz, DMSO-d₆) δ ppm 1.61-1.72 (1H, m), 1.73-1.90 (1H, m), 1.99 (3H, s), 2.61-2.89 (1H, m), 3.10 (3H, d, J=4.65 Hz), 3.14-3.26 (1H, m), 3.83-3.98 (1H, m), 4.00-4.19 (1H, m), 4.39-4.68 (1H, m), 4.94 (1H, d, J=49.50 Hz), 5.02 (2H, dt, J=22.75, 4.65 Hz), 5.09 (2H, s), 6.40 (1H, d, J=7.94 Hz), 7.65 (1H, d, J=2.19 Hz), 7.93 (1H, d, J=9.03 Hz), 8.66 (1H, d, J=9.04 Hz), 10.28 (1H, q, J=4.56 Hz); ESIMS found for C₂₁H₂₃F₃N₁₀O m/z 489.2 (M+1).

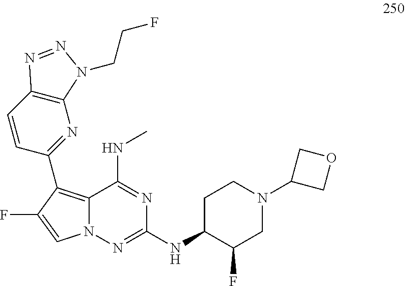 [SMILES: CNc1nc(N[C@H]2CCN(C3COC3)C[C@H]2F)nn2cc(F)c(-c3ccc4nnn(CCF)c4n3)c12]

6-Fluoro-N²-((3R,4S)-3-fluoro-1-(oxetan-3-yl)piperidin-4-yl)-5-(3-(2-fluoroethyl)-3H-[1,2,3]triazolo[4,5-b]pyridin-5-yl)-N⁴-methylpyrrolo[2,1-f][1,2,4]triazine-2,4-diamine 250.

Fluffy yellow solid (15 mg, 0.0300 mmol, 83.0% yield). ¹H NMR (499 MHz, DMSO-d₆) δ ppm 1.64-1.76 (1H, m), 1.89 (1H, qd, J=12.23, 3.83 Hz), 1.96-2.03 (1H, m), 2.13 (1H, dd, J=37.00, 12.32 Hz), 2.69-2.81 (1H, m), 2.91-3.03 (1H, m), 3.09 (3H, d, J=4.65 Hz), 3.48 (1H, quin, J=6.23 Hz), 3.71-3.88 (1H, m), 4.39 (1H, t, J=6.16 Hz), 4.45 (1H, t, J=6.16 Hz), 4.54 (2H, td, J=6.50, 3.15 Hz), 4.89 (1H, d, J=49.35 Hz), 5.01 (2H, dt, J=22.60, 5.04 Hz), 5.09 (2H, s), 6.29 (1H, d, J=7.94 Hz), 7.65 (1H, d, J=2.19 Hz), 7.93 (1H, d, J=8.76 Hz), 8.65 (1H, d, J=9.03 Hz), 10.26 (1H, q, J=4.56 Hz); ESIMS found for C₂₂H₂₅F₃N₁₀O m/z 503.2 (M+1).

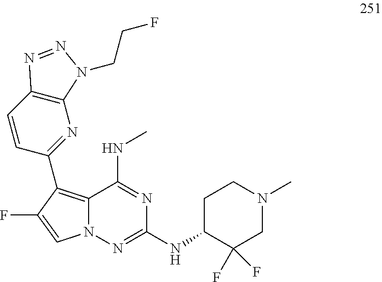 [SMILES: CNc1nc(N[C@@H]2CCN(C)CC2(F)F)nn2cc(F)c(-c3ccc4nnn(CCF)c4n3)c12]

(R)—N²-(3,3-Difluoro-1-methylpiperidin-4-yl)-6-fluoro-5-(3-(2-fluoroethyl)-3H-[1,2,3]triazolo[4,5-b]pyridin-5-yl)-N⁴-methylpyrrolo[2,1-f][1,2,4]triazine-2,4-diamine 251.

Fluffy yellow solid (12 mg, 0.025 mmol, 89.7% yield). ¹H NMR (499 MHz, DMSO-d₆) δ ppm 1.73-1.89 (2H, m), 2.15 (1H, td, J=11.16, 2.60 Hz), 2.25 (3H, s), 2.35 (1H, ddd, J=26.35, 12.10, 1.40 Hz), 2.77 (1H, br d, J=11.77 Hz), 2.98-3.08 (1H, m), 3.10 (3H, d, J=4.65 Hz), 4.12-4.31 (1H, m), 5.02 (2H, dt, J=23.85, 4.65 Hz), 5.09 (2H, s), 6.45 (1H, d, J=9.58 Hz), 7.66 (1H, d, J=2.19 Hz), 7.93 (1H, d, J=9.03 Hz), 8.65 (1H, d, J=9.03 Hz), 10.28 (1H, q, J=4.56 Hz); ESIMS found for C₂₀H₂₂F₄N₁₀ m/z 479.2 (M+1).

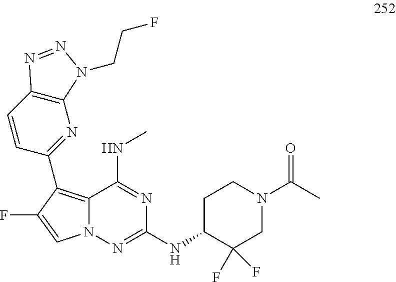 [SMILES: CNc1nc(N[C@@H]2CCN(C(C)=O)CC2(F)F)nn2cc(F)c(-c3ccc4nnn(CCF)c4n3)c12]

(R)-1-(3,3-Difluoro-4-((6-fluoro-5-(3-(2-fluoroethyl)-3H-[1,2,3]triazolo[4,5-b]pyridin-5-yl)-4-(methylamino)pyrrolo[2,1-f][1,2,4]triazin-2-yl)amino)piperidin-1-yl)ethan-1-one 252.

Fluffy yellow solid (9 mg, 0.018 mmol, 74.9% yield). ¹H NMR (499 MHz, DMSO-d₆) δ ppm 1.55-1.80 (1H, m), 1.82-1.98 (1H, m), 2.07 (3H, s), 2.92-3.05 (1H, m), 3.12 (3H, d, J=4.65 Hz), 3.60-3.92 (1H, m), 4.08-4.30 (1H, m), 4.40-4.59 (2H, m), 5.02 (2H, dt, J=24.40, 4.65 Hz), 5.09 (2H, s), 6.65 (1H, br d, J=9.31 Hz), 7.65 (1H, s), 7.94 (1H, d, J=8.76 Hz), 8.66 (1H, d, J=8.76 Hz), 10.30 (1H, q, J=4.65 Hz); ESIMS found for C₂₁H₂₂F₄N₁₀O m/z 507.2 (M+1).

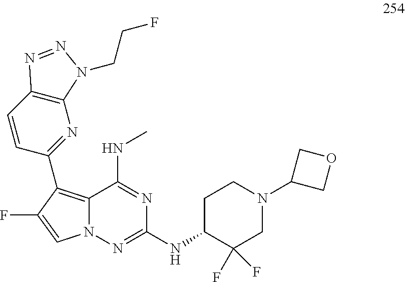 [SMILES: CNc1nc(N[C@@H]2CCN(C3COC3)CC2(F)F)nn2cc(F)c(-c3ccc4nnn(CCF)c4n3)c12]

(R)—N²-(3,3-Difluoro-1-(oxetan-3-yl)piperidin-4-yl)-6-fluoro-5-(3-(2-fluoroethyl)-3H-[1,2,3]triazolo[4,5-b]pyridin-5-yl)-N⁴-methylpyrrolo[2,1-f][1,2,4]triazine-2,4-diamine 254.

Fluffy yellow solid (12 mg, 0.023 mmol, 83.6% yield). ¹H NMR (499 MHz, DMSO-d₆) δ ppm 1.73-1.83 (1H, m), 1.84-1.90 (1H, m), 2.08-2.20 (1H, m), 2.37 (1H, dd, J=27.40, 11.25 Hz), 2.75 (1H, br d, J=11.77 Hz), 2.95-3.06 (1H, m), 3.11 (3H, d, J=4.65 Hz), 3.60 (1H, quin, J=6.30 Hz), 4.21-4.36 (1H, m), 4.43 (2H, dt, J=14.78, 6.16 Hz), 4.55 (2H, td, J=6.57, 3.83 Hz), 5.02 (2H, dt, J=23.85, 4.70 Hz), 5.09 (2H, s), 6.53 (1H, d, J=9.31 Hz), 7.66 (1H, d, J=2.19 Hz), 7.93 (1H, d, J=9.03 Hz), 8.65 (1H, d, J=8.76 Hz), 10.29 (1H, q, J=4.80 Hz); ESIMS found for C₂₂H₂₄F₄N₁₀O m/z 521.2 (M+1).

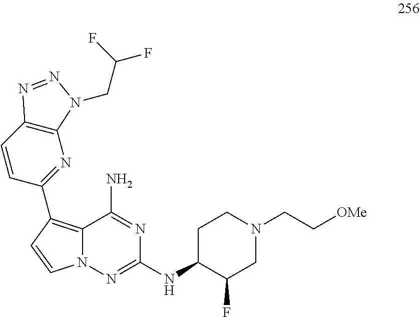 [SMILES: COCCN1CC[C@H](Nc2nc(N)c3c(-c4ccc5nnn(CC(F)F)c5n4)ccn3n2)[C@H](F)C1]

5-(3-(2,2-Difluoroethyl)-3H-[1,2,3]triazolo[4,5-b]pyridin-5-yl)-N²-((3R,4S)-3-fluoro-1-(2-methoxyethyl)piperidin-4-yl)pyrrolo[2,1-f][1,2,4]triazine-2,4-diamine 256.

Light yellow solid (22 mg, 0.045 mmol, 51.3% yield). ¹H NMR (499 MHz, DMSO-d₆) δ ppm 1.60-1.72 (1H, m), 1.84 (1H, qd, J=12.23, 3.83 Hz), 2.16 (1H, br t, J=10.81 Hz), 2.29 (1H, dd, J=37.85, 12.59 Hz), 2.52 (2H, t, J=5.75 Hz), 2.89 (1H, br d, J=12.32 Hz), 3.09-3.19 (1H, m), 3.24 (3H, s), 3.43 (2H, t, J=5.75 Hz), 3.68-3.86 (1H, m), 4.84 (1H, d, J=49.95 Hz), 5.16 (2H, td, J=16.15, 2.19 Hz), 6.00 (1H, d, J=7.94 Hz), 6.58 (1H, tt, J=53.75, 2.46 Hz), 7.34 (1H, d, J=3.29 Hz), 7.46 (1H, d, J=3.01 Hz), 7.69 (1H, br s), 8.09 (1H, d, J=9.31 Hz), 8.54 (1H, d, J=9.03 Hz), 10.46 (1H, br s); ESIMS found for C₂₁H₂₅F₃N₁₀O m/z 491.2 (M+1).

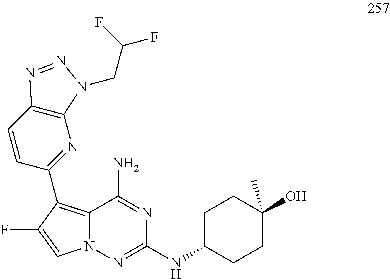 [SMILES: C[C@]1(O)CC[C@H](Nc2nc(N)c3c(-c4ccc5nnn(CC(F)F)c5n4)c(F)cn3n2)CC1]

(1r,4r)-4-((4-Amino-5-(3-(2,2-difluoroethyl)-3H-[1,2,3]triazolo[4,5-b]pyridin-5-yl)-6-fluoropyrrolo[2,1-f][1,2,4]triazin-2-yl)amino)-1-methylcyclohexan-1-ol 257.

Yellow solid (15 mg, 0.033 mmol, 31.5% yield). ¹H NMR (499 MHz, DMSO-d₆) δ ppm 1.13 (3H, s), 1.33-1.48 (4H, m), 1.53-1.64 (2H, m), 1.76-1.88 (2H, m), 3.55-3.69 (1H, m), 4.21 (1H, s), 5.19 (2H, td, J=16.15, 2.46 Hz), 6.05 (1H, d, J=8.21 Hz), 6.60 (2H, tt, J=53.80, 2.75 Hz), 7.62 (1H, br s), 7.68 (1H, d, J=2.19 Hz), 7.93 (1H, d, J=9.03 Hz), 8.65 (1H, d, J=8.76 Hz), 9.64 (1H, br s); ESIMS found for C₂₀H₂₂F₃N₉O m/z 462.2 (M+1).

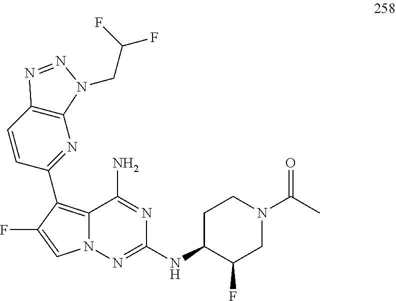 [SMILES: CC(=O)N1CC[C@H](Nc2nc(N)c3c(-c4ccc5nnn(CC(F)F)c5n4)c(F)cn3n2)[C@H](F)C1]

1-((3R,4S)-4-((4-Amino-5-(3-(2,2-difluoroethyl)-3H-[1,2,3]triazolo[4,5-b]pyridin-5-yl)-6-fluoropyrrolo[2,1-f][1,2,4]triazin-2-yl)amino)-3-fluoropiperidin-1-yl)ethan-1-one 258.

Fluffy yellow solid (6 mg, 0.012 mmol, 94.8% yield). ¹H NMR (499 MHz, DMSO-d₆) δ ppm 1.59-1.69 (1H, m), 1.71-1.87 (1H, m), 1.99 (3H, s), 2.59-2.73 (1H, m), 3.09-3.24 (1H, m), 3.82-3.96 (1H, m), 3.98-4.17 (1H, m), 4.41-4.72 (1H, m), 4.93 (1H, d, J=49.90 Hz), 5.19 (2H, td, J=16.22, 2.33 Hz), 6.31 (1H, d, J=8.21 Hz), 6.60 (2H, tt, J=54.00, 2.60 Hz), 7.68 (1H, d, J=1.92 Hz), 7.75 (1H, br s), 7.94 (1H, d, J=8.76 Hz), 8.67 (1H, d, J=9.03 Hz), 9.71 (1H, br s); ESIMS found for C₂₀H₂₀F₄N₁₀O m/z 493.15 (M+1).

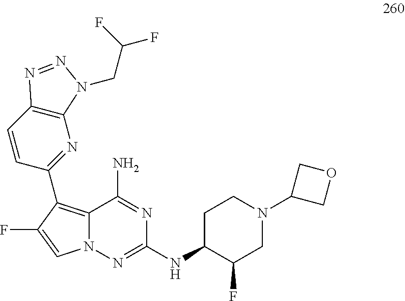 [SMILES: Nc1nc(N[C@H]2CCN(C3COC3)C[C@H]2F)nn2cc(F)c(-c3ccc4nnn(CC(F)F)c4n3)c12]

5-(3-(2,2-Difluoroethyl)-3H-[1,2,3]triazolo[4,5-b]pyridin-5-yl)-6-fluoro-N²-((3R,4S)-3-fluoro-1-(oxetan-3-yl)piperidin-4-yl)pyrrolo[2,1-f][1,2,4]triazine-2,4-diamine 260.

Yellow fluffy solid (10 mg, 0.0120 mmol, 18.2% yield). ¹H NMR (499 MHz, DMSO-d₆) δ ppm 1.70 (1H, br dd, J=8.62, 3.97 Hz), 1.87 (1H, qd, J=12.09, 3.42 Hz), 1.94-2.02 (1H, m), 2.11 (1H, dd, J=37.05, 12.59 Hz), 2.74 (1H, br d, J=12.05 Hz), 2.90-3.03 (1H, m), 3.48 (1H, quin, J=6.43 Hz), 3.71-3.89 (1H, m), 4.39 (1H, t, J=6.16 Hz), 4.45 (1H, t, J=6.16 Hz), 4.53 (2H, td, J=6.50, 2.60 Hz), 4.86 (1H, d, J=49.35 Hz), 5.19 (2H, td, J=16.08, 2.33 Hz), 6.19 (1H, d, J=8.21 Hz), 7.68 (1H, d, J=2.19 Hz), 7.74 (1H, br s), 7.94 (1H, d, J=9.03 Hz), 8.67 (1H, d, J=9.03 Hz), 9.70 (1H, br s); ESIMS found for C₂₁H₂₂F₄N₁₀O m/z 507.2 (M+1).

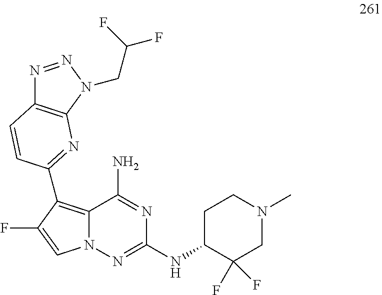 [SMILES: CN1CC[C@@H](Nc2nc(N)c3c(-c4ccc5nnn(CC(F)F)c5n4)c(F)cn3n2)C(F)(F)C1]

(R)—N²-(3,3-Difluoro-1-methylpiperidin-4-yl)-5-(3-(2,2-difluoroethyl)-3H-[1,2,3]triazolo[4,5-b]pyridin-5-yl)-6-fluoropyrrolo[2,1-f][1,2,4]triazine-2,4-diamine 261.

Fluffy yellow solid (2.8 mg, 0.006 mmol, 42.5% yield). ¹H NMR (499 MHz, DMSO-d₆) δ ppm 1.71-1.87 (2H, m), 2.06-2.19 (1H, m), 2.25 (3H, s), 2.31 (1H, dd, J=26.85, 11.77 Hz), 2.78 (1H, br d, J=12.05 Hz), 3.00-3.10 (1H, m), 4.13-4.30 (1H, m), 5.19 (2H, td, J=16.08, 2.05 Hz), 6.38 (1H, br d, J=9.58 Hz), 6.60 (1H, tt, J=54.05, 2.55 Hz), 7.69 (1H, d, J=1.92 Hz), 7.79 (1H, br s), 7.94 (1H, d, J=8.76 Hz), 8.67 (1H, d, J=9.03 Hz), 9.73 (1H, br s); ESIMS found for C₁₉H₁₉F₅N₁₀ m/z 483.2 (M+1).

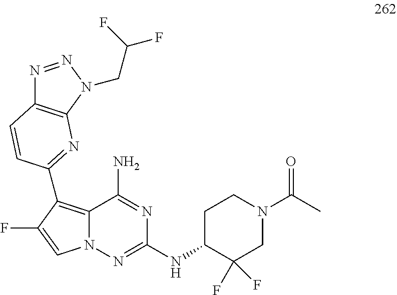 [SMILES: CC(=O)N1CC[C@@H](Nc2nc(N)c3c(-c4ccc5nnn(CC(F)F)c5n4)c(F)cn3n2)C(F)(F)C1]

(R)-1-(4-((4-Amino-5-(3-(2,2-difluoroethyl)-3H-[1,2,3]triazolo[4,5-b]pyridin-5-yl)-6-fluoropyrrolo[2,1-f][1,2,4]triazin-2-yl)amino)-3,3-difluoropiperidin-1-yl)ethan-1-one 262.

Yellow fluffy solid (4 mg, 0.008 mmol, 58.0% yield). ¹H NMR (499 MHz, DMSO-d₆) δ ppm 1.57-1.79 (1H, m), 1.81-1.95 (1H, m), 2.06 (3H, s), 2.91-3.03 (1H, m), 3.64-3.90 (1H, m), 4.09-4.28 (1H, m), 4.41-4.57 (2H, m), 5.20 (2H, td, J=16.15, 2.46 Hz), 6.60 (1H, tt, J=54.00, 2.74 Hz), 6.59 (1H, d, J=8.10 Hz), 7.68 (1H, d, J=1.92 Hz), 7.79 (1H, br s), 7.95 (1H, d, J=8.76 Hz), 8.68 (1H, d, J=8.76 Hz), 9.75 (1H, br s); ESIMS found for C₂₀H₁₉F₅N₁₀O m/z 511.1 (M+1).

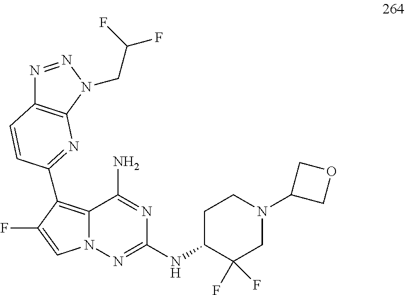 [SMILES: Nc1nc(N[C@@H]2CCN(C3COC3)CC2(F)F)nn2cc(F)c(-c3ccc4nnn(CC(F)F)c4n3)c12]

(R)—N²-(3,3-Difluoro-1-(oxetan-3-yl)piperidin-4-yl)-5-(3-(2,2-difluoroethyl)-3H-[1,2,3]triazolo[4,5-b]pyridin-5-yl)-6-fluoropyrrolo[2,1-f][1,2,4]triazine-2,4-diamine 264.

Yellow fluffy solid (7 mg, 0.013 mmol, 93.7% yield). ¹H NMR (499 MHz, DMSO-d₆) δ ppm 1.70-1.82 (1H, m), 1.83-1.91 (1H, m), 2.06-2.16 (1H, m), 2.33 (1H, dd, J=27.15, 11.23 Hz), 2.75 (1H, br d, J=11.77 Hz), 2.96-3.06 (1H, m), 3.59 (1H, quin, J=6.23 Hz), 4.19-4.34 (1H, m), 4.43 (2H, dt, J=14.24, 6.30 Hz), 4.54 (2H, td, J=6.57, 3.83 Hz), 5.19 (2 H, td, J=16.15, 2.46 Hz), 6.46 (1H, br d, J=9.31 Hz), 6.60 (2H, tt, J=54.00, 2.60 Hz), 7.69 (1H, d, J=2.19 Hz), 7.78 (1H, br s), 7.94 (1H, d, J=9.03 Hz), 8.67 (1H, d, J=8.76 Hz), 9.74 (1H, br s); ESIMS found for C₂₁H₂₁F₅N₁₀O m/z 525.2 (M+1).

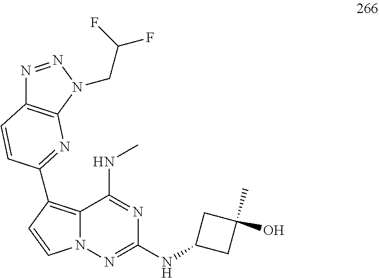 [SMILES: CNc1nc(N[C@H]2C[C@](C)(O)C2)nn2ccc(-c3ccc4nnn(CC(F)F)c4n3)c12]

(1r,3r)-3-((5-(3-(2,2-Difluoroethyl)-3H-[1,2,3]triazolo[4,5-b]pyridin-5-yl)-4-(methylamino)pyrrolo[2,1-f][1,2,4]triazin-2-yl)amino)-1-methylcyclobutan-1-ol 266.

Yellow solid (16 mg, 0.037 mmol, 35.0% yield). ¹H NMR (499 MHz, DMSO-d₆) δ ppm 1.27 (3H, s), 1.92-2.01 (2H, m), 2.24-2.33 (2H, m), 3.12 (3H, d, J=4.65 Hz), 4.28 (1H, sxt, J=7.56 Hz), 4.74 (1H, s), 5.26 (2H, td, J=16.08, 2.60 Hz), 6.51 (1H, d, J=7.12 Hz), 6.72 (1H, tt, J=54.00, 2.87 Hz), 7.32 (1H, d, J=3.01 Hz), 7.43 (1H, d, J=2.74 Hz), 8.10 (1H, d, J=9.31 Hz), 8.54 (1H, d, J=9.04 Hz), 10.87 (1H, q, J=4.56 Hz); ESIMS found for C₁₉H₂₁F₂N₉O m/z 430.2 (M+1).

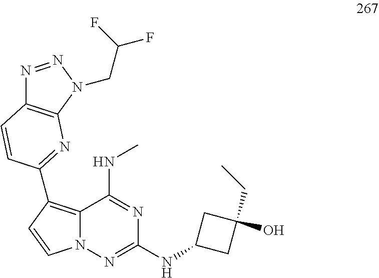 [SMILES: CC[C@]1(O)C[C@H](Nc2nc(NC)c3c(-c4ccc5nnn(CC(F)F)c5n4)ccn3n2)C1]

(1r,3s)-3-((5-(3-(2,2-Difluoroethyl)-3H-[1,2,3]triazolo[4,5-b]pyridin-5-yl)-4-(methylamino)pyrrolo[2,1-f][1,2,4]triazin-2-yl)amino)-1-ethylcyclobutan-1-ol 267.

Yellow solid (32 mg, 0.072 mmol, 54.7% yield). ¹H NMR (499 MHz, DMSO-d₆) δ ppm 0.81 (3H, t, J=7.39 Hz), 1.50 (2H, q, J=7.39 Hz), 1.86-1.97 (2H, m), 2.17-2.26 (2H, m), 3.12 (3H, d, J=4.65 Hz), 4.32 (1H, sxt, J=7.67 Hz), 4.57 (1H, s), 5.26 (2H, td, J=15.88, 2.46 Hz), 6.50 (1H, d, J=7.12 Hz), 6.72 (1H, tt, J=54.00, 2.87 Hz), 7.32 (1H, d, J=3.01 Hz), 7.43 (1H, d, J=3.01 Hz), 8.10 (1H, d, J=9.31 Hz), 8.54 (1H, d, J=9.04 Hz), 10.87 (1H, q, J=4.65 Hz); ESIMS found for C₂₀H₂₃F₂N₉O m/z 444.2 (M+1).

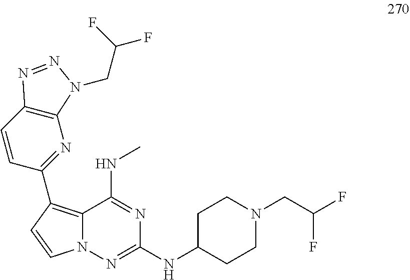 [SMILES: CNc1nc(NC2CCN(CC(F)F)CC2)nn2ccc(-c3ccc4nnn(CC(F)F)c4n3)c12]

5-(3-(2,2-Difluoroethyl)-3H-[1,2,3]triazolo[4,5-b]pyridin-5-yl)-N²-(1-(2,2-difluoroethyl)piperidin-4-yl)-N⁴-methylpyrrolo[2,1-f][1,2,4]triazine-2,4-diamine 270.

Yellow solid (54 mg, 0.110 mmol, 96.4% yield). ¹H NMR (499 MHz, DMSO-d₆) δ ppm 1.46-1.59 (2H, m), 1.87 (2H, br d, J=9.86 Hz), 2.19-2.30 (2H, m), 2.71 (2H, td, J=15.74, 4.38 Hz), 2.89 (2H, br d, J=11.77 Hz), 3.12 (3H, d, J=4.65 Hz), 3.53-3.64 (1H, m), 5.25 (2H, td, J=16.08, 2.60 Hz), 6.13 (1H, tt, J=55.95, 4.38 Hz), 6.17 (1H, d, J=8.21 Hz), 6.72 (1H, tt, J=54.00, 2.87 Hz), 7.32 (1H, d, J=3.01 Hz), 7.44 (1H, d, J=3.01 Hz), 8.10 (1H, d, J=9.03 Hz), 8.54 (1H, d, J=9.03 Hz), 10.87 (1H, q, J=4.47 Hz); ESIMS found for C₂₁H₂₄F₄N₁₀ m/z 493.2 (M+1).

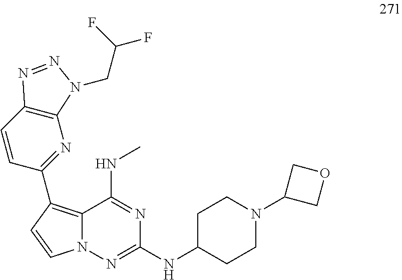 [SMILES: CNc1nc(NC2CCN(C3COC3)CC2)nn2ccc(-c3ccc4nnn(CC(F)F)c4n3)c12]

5-(3-(2,2-Difluoroethyl)-3H-[1,2,3]triazolo[4,5-b]pyridin-5-yl)-N⁴-methyl-N²-(1-(oxetan-3-yl)piperidin-4-yl)pyrrolo[2,1-f][1,2,4]triazine-2,4-diamine 271.

Yellow solid (5 mg, 0.010 mmol, 27.6% yield). ¹H NMR (499 MHz, DMSO-d₆) δ ppm 1.45-1.59 (2H, m), 1.81-1.87 (2H, m), 1.89 (2H, br d, J=11.77 Hz), 2.68 (2H, br d, J=10.95 Hz), 3.12 (3H, d, J=4.65 Hz), 3.54-3.65 (1H, m), 4.42 (2H, t, J=6.16 Hz), 4.52 (2H, t, J=6.57 Hz), 5.25 (2H, td, J=16.02, 2.46 Hz), 6.19 (1H, d, J=8.21 Hz), 6.71 (1H, tt, J=54.05, 2.87 Hz), 7.31 (1H, d, J=3.01 Hz), 7.43 (1H, d, J=3.01 Hz), 8.10 (1H, d, J=9.31 Hz), 8.54 (1H, d, J=9.03 Hz), 10.87 (1H, q, J=4.65 Hz); ESIMS found for C₂₂H₂₆F₂N₁₀O m/z 485.2 (M+1).

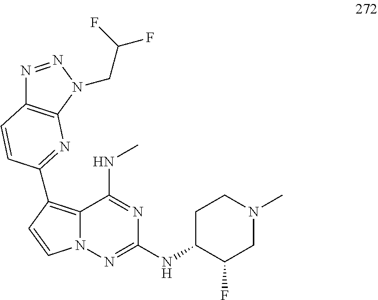 [SMILES: CNc1nc(N[C@@H]2CCN(C)C[C@@H]2F)nn2ccc(-c3ccc4nnn(CC(F)F)c4n3)c12]

5-(3-(2,2-Difluoroethyl)-3H-[1,2,3]triazolo[4,5-b]pyridin-5-yl)-N²-((3S,4R)-3-fluoro-1-methylpiperidin-4-yl)-N⁴-methylpyrrolo[2,1-f][1,2,4]triazine-2,4-diamine 272.

Yellow solid (30 mg, 0.065 mmol, 34.6% yield). ¹H NMR (499 MHz, DMSO-d₆) δ ppm 1.63-1.74 (1H, m), 1.90 (1H, qd, J=12.23, 3.83 Hz), 2.02-2.14 (1H, m), 2.22 (1H, dd, J=37.85, 13.14 Hz), 2.21 (3H, s), 2.82 (1H, br d, J=11.50 Hz), 3.02-3.10 (1H, m), 3.13 (3H, d, J=4.93 Hz), 3.69-3.85 (1H, m), 4.89 (1H, d, J=49.90 Hz), 5.26 (2H, td, J=16.02, 2.46 Hz), 6.11 (1H, d, J=7.67 Hz), 6.72 (1H, tt, J=54.30, 2.88 Hz), 7.34 (1H, d, J=2.74 Hz), 7.44 (1H, d, J=2.74 Hz), 8.11 (1H, d, J=8.76 Hz), 8.55 (1H, d, J=8.76 Hz), 10.92 (1H, q, J=4.56 Hz); ESIMS found for C₂₀H₂₃F₃N₁₀ m/z 461.2 (M+1).

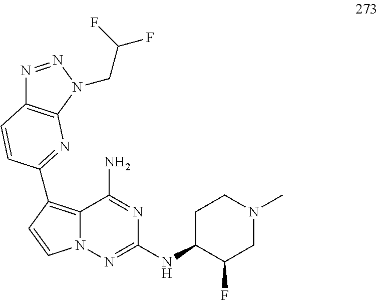 [SMILES: CN1CC[C@H](Nc2nc(N)c3c(-c4ccc5nnn(CC(F)F)c5n4)ccn3n2)[C@H](F)C1]

5-(3-(2,2-Difluoroethyl)-3H-[1,2,3]triazolo[4,5-b]pyridin-5-yl)-N²-((3R,4S)-3-fluoro-1-methylpiperidin-4-yl)-N⁴-methylpyrrolo[2,1-f][1,2,4]triazine-2,4-diamine 273.

Yellow solid (4 mg, 0.009 mmol, 38.8% yield). ¹H NMR (499 MHz, DMSO-d₆) δ ppm 1.62-1.74 (1H, m), 1.89 (1H, qd, J=12.23, 3.83 Hz), 2.04 (1H, br t, J=10.95 Hz), 2.16 (1H, dd, J=37.30, 12.87 Hz), 2.18 (3H, s), 2.79 (1H, br d, J=10.68 Hz), 2.97-3.08 (1H, m), 3.13 (3H, d, J=4.65 Hz), 3.68-3.84 (1H, m), 4.88 (1H, d, J=49.90 Hz), 5.26 (2H, td, J=16.08, 2.60 Hz), 6.09 (1H, d, J=7.94 Hz), 6.72 (1H, tt, J=54.05, 2.87 Hz), 7.34 (1H, d, J=3.01 Hz), 7.44 (1H, d, J=3.01 Hz), 8.11 (1H, d, J=9.03 Hz), 8.55 (1H, d, J=9.04 Hz), 10.92 (1H, q, J=4.65 Hz); ESIMS found for C₂₀H₂₃F₃N₁₀ m/z 461.2 (M+1).

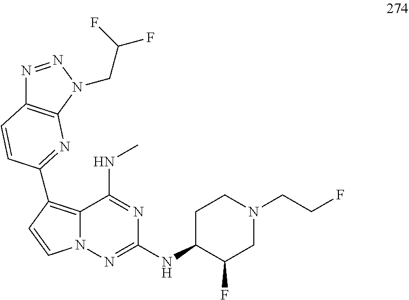 [SMILES: CNc1nc(N[C@H]2CCN(CCF)C[C@H]2F)nn2ccc(-c3ccc4nnn(CC(F)F)c4n3)c12]

5-(3-(2,2-Difluoroethyl)-3H-[1,2,3]triazolo[4,5-b]pyridin-5-yl)-N²-((3R,4S)-3-fluoro-1-(2-fluoroethyl)piperidin-4-yl)-N⁴-methylpyrrolo[2,1-f][1,2,4]triazine-2,4-diamine 274.

Yellow solid (55 mg, 0.112 mmol, 93.2% yield). ¹H NMR (499 MHz, DMSO-d₆) δ ppm 1.64-1.74 (1H, m), 1.89 (1H, qd, J=12.23, 4.38 Hz), 2.18-2.28 (1H, m), 2.37 (1H, dd, J=37.45, 12.59 Hz), 2.67 (2H, dt, J=28.55, 4.93 Hz), 2.92 (1H, br d, J=9.86 Hz), 3.13 (3H, d, J=4.38 Hz), 3.14-3.20 (1H, m), 3.71-3.87 (1H, m), 4.54 (2H, dt, J=48.30, 4.93 Hz), 4.89 (1H, d, J=49.50 Hz), 5.26 (2H, td, J=16.15, 2.74 Hz), 6.11 (1H, d, J=8.21 Hz), 6.72 (1H, tt, J=53.75, 2.88 Hz), 7.34 (1H, d, J=3.29 Hz), 7.44 (1H, d, J=3.29 Hz), 8.11 (1H, d, J=9.31 Hz), 8.55 (1H, d, J=9.31 Hz), 10.93 (1H, q, J=4.38 Hz); ESIMS found for C₂₁H₂₄F₄N₁₀ m/z 493.2 (M+1).

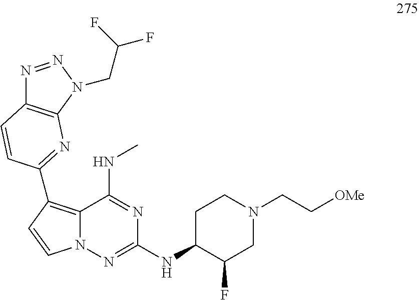 [SMILES: CNc1nc(N[C@H]2CCN(CCOC)C[C@H]2F)nn2ccc(-c3ccc4nnn(CC(F)F)c4n3)c12]

5-(3-(2,2-Difluoroethyl)-3H-[1,2,3]triazolo[4,5-b]pyridin-5-yl)-N²-((3R,4S)-3-fluoro-1-(2-methoxyethyl)piperidin-4-yl)-N⁴-methylpyrrolo[2,1-f][1,2,4]triazine-2,4-diamine 275.

Yellow solid (7 mg, 0.014 mmol, 75.6% yield). ¹H NMR (499 MHz, DMSO-d₆) δ ppm 1.68 (1H, br d, J=6.02 Hz), 1.80-1.94 (1H, m), 2.10-2.21 (1H, m), 2.23-2.36 (1H, m), 2.51-2.55 (2H, m), 2.86-2.95 (1H, m), 3.13 (3H, d, J=4.38 Hz), 3.13-3.19 (1H, m), 3.25 (3H, s), 3.44 (2H, br s), 3.72-3.87 (1H, m), 4.87 (1H, d, J=50.50 Hz), 5.26 (2H, td, J=15.88, 2.74 Hz), 6.02-6.14 (1H, m), 6.72 (1H, tt, J=54.00, 2.87 Hz), 7.34 (1H, d, J=3.29 Hz), 7.44 (1H, d, J=2.74 Hz), 8.11 (1H, d, J=9.31 Hz), 8.55 (1H, d, J=8.76 Hz), 10.93 (1H, q, J=4.65 Hz); ESIMS found for C₂₂H₂₇F₃N₁₀O m/z 505.2 (M+1).

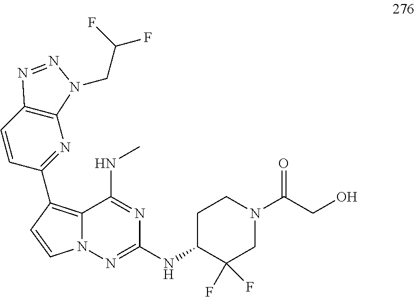 [SMILES: CNc1nc(N[C@@H]2CCN(C(=O)CO)CC2(F)F)nn2ccc(-c3ccc4nnn(CC(F)F)c4n3)c12]

(R)-1-(4-((5-(3-(2,2-Difluoroethyl)-3H-[1,2,3]triazolo[4,5-b]pyridin-5-yl)-4-(methylamino)pyrrolo[2,1-f][1,2,4]triazin-2-yl)amino)-3,3-difluoropiperidin-1-yl)-2-hydroxyethan-1-one 276.

Fluffy yellow solid (27 mg, 0.052 mmol, 74.5% yield). ¹H NMR (499 MHz, DMSO-d₆) δ ppm 1.57-1.81 (1H, m), 1.84-1.98 (1H, m), 3.06 (1H, br t, J=11.36 Hz), 3.16 (3H, d, J=4.65 Hz), 3.56-3.84 (1H, m), 4.03-4.25 (3H, m), 4.41-4.51 (1H, m), 4.51-4.61 (1H, m), 4.77-4.86 (1H, m), 5.27 (2H, td, J=16.02, 2.19 Hz), 6.56 (1H, br d, J=9.58 Hz), 6.72 (1H, tt, J=54.00, 2.74 Hz), 7.36 (1H, d, J=3.01 Hz), 7.44 (1H, d, J=3.01 Hz), 8.13 (1H, d, J=9.31 Hz), 8.56 (1H, d, J=9.04 Hz), 10.97 (1H, q, J=4.65 Hz); ESIMS found for C₂₁H₂₂F₄N₁₀O₂ m/z 523.15 (M+1).

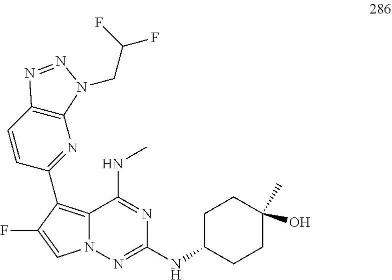 [SMILES: CNc1nc(N[C@H]2CC[C@](C)(O)CC2)nn2cc(F)c(-c3ccc4nnn(CC(F)F)c4n3)c12]

(1r,4r)-4-((5-(3-(2,2-Difluoroethyl)-3H-[1,2,3]triazolo[4,5-b]pyridin-5-yl)-6-fluoro-4-(methylamino)pyrrolo[2,1-f][1,2,4]triazin-2-yl)amino)-1-methylcyclohexan-1-ol 286.

Fluffy yellow solid (2 mg, 0.004 mmol, 40.1% yield). ¹H NMR (499 MHz, DMSO-d₆) δ ppm 1.14 (3H, s), 1.38-1.48 (4H, m), 1.55-1.63 (2H, m), 1.80-1.87 (2H, m), 3.08 (3H, d, J=4.93 Hz), 3.56-3.69 (1H, m), 4.22 (1H, s), 5.26 (2H, td, J=16.15, 2.74 Hz), 6.16 (1H, d, J=8.21 Hz), 6.71 (1H, tt, J=53.75, 2.87 Hz), 7.67 (1H, d, J=1.92 Hz), 7.95 (1H, d, J=9.03 Hz), 8.66 (1H, d, J=8.76 Hz), 10.14 (1H, q, J=4.47 Hz); ESIMS found for C₂₁H₂₄F₃N₉O m/z 476.2 (M+1).

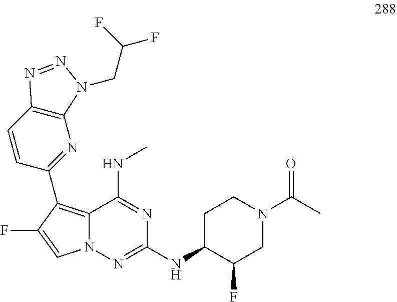 [SMILES: CNc1nc(N[C@H]2CCN(C(C)=O)C[C@H]2F)nn2cc(F)c(-c3ccc4nnn(CC(F)F)c4n3)c12]

1-((3R,4S)-4-((5-(3-(2,2-Difluoroethyl)-3H-[1,2,3]triazolo[4,5-b]pyridin-5-yl)-6-fluoro-4-(methylamino)pyrrolo[2,1-f][1,2,4]triazin-2-yl)amino)-3-fluoropiperidin-1-yl)ethan-1-one 288.

Fluffy yellow solid (4 mg, 0.008 mmol, 91.7% yield). ¹H NMR (499 MHz, DMSO-d₆) δ ppm 1.60-1.72 (1H, m), 1.73-1.88 (1H, m), 1.97-2.06 (3H, m), 2.60-2.73 (1H, m), 2.87 (1H, dd, J=39.25, 13.96 Hz), 3.10 (3H, d, J=4.65 Hz), 3.84-3.97 (1H, m), 4.00-4.17 (1H, m), 4.40-4.72 (1H, m), 4.95 (1H, d, J=49.35 Hz), 5.27 (2H, td, J=16.02, 2.74 Hz), 6.42 (1H, d, J=7.94 Hz), 6.72 (2H, tt, J=54.00, 2.87 Hz), 7.65 (1H, d, J=2.19 Hz), 7.96 (1H, d, J=9.03 Hz), 8.68 (1H, d, J=9.03 Hz), 10.16-10.27 (1H, m); ESIMS found for C₂₁H₂₂F₄N₁₀O m/z 507.2 (M+1).

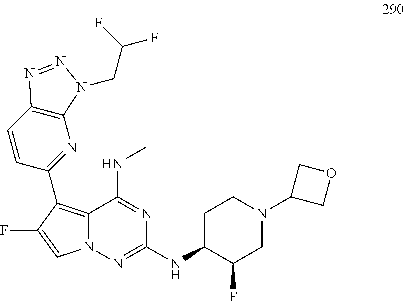 [SMILES: CNc1nc(N[C@H]2CCN(C3COC3)C[C@H]2F)nn2cc(F)c(-c3ccc4nnn(CC(F)F)c4n3)c12]

5-(3-(2,2-Difluoroethyl)-3H-[1,2,3]triazolo[4,5-b]pyridin-5-yl)-6-fluoro-N²-((3R,4S)-3-fluoro-1-(oxetan-3-yl)piperidin-4-yl)-N⁴-methylpyrrolo[2,1-f][1,2,4]triazine-2,4-diamine 290.

Fluffy yellow solid (13 mg, 0.025 mmol, 72.2% yield). ¹H NMR (499 MHz, DMSO-d₆) δ ppm 1.63-1.76 (1H, m), 1.88 (1H, qd, J=12.05, 3.56 Hz), 1.95-2.04 (1H, m), 2.13 (1H, dd, J=37.30, 12.59 Hz), 2.75 (1H, br d, J=9.31 Hz), 2.90-3.03 (1H, m), 3.09 (3H, d, J=4.65 Hz), 3.48 (1H, quin, J=6.43 Hz), 3.72-3.89 (1H, m), 4.39 (1H, t, J=6.16 Hz), 4.45 (1H, t, J=6.16 Hz), 4.54 (2H, td, J=6.57, 3.01 Hz), 4.89 (1H, d, J=49.60 Hz), 5.27 (2H, td, J=16.08, 2.87 Hz), 6.30 (1H, d, J=7.94 Hz), 6.72 (1H, tt, J=54.05, 2.87 Hz), 7.65 (1H, d, J=2.19 Hz), 7.95 (1H, d, J=9.03 Hz), 8.67 (1H, d, J=8.76 Hz), 10.20 (1H, q, J=4.56 Hz) ESIMS found for C₂₂H₂₄F₄N₁₀O m/z 521.2 (M+1).

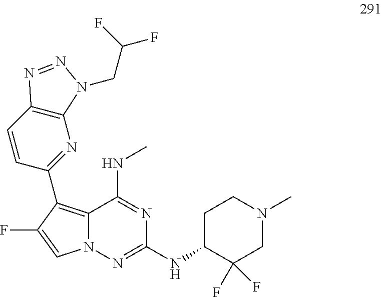 [SMILES: CNc1nc(N[C@@H]2CCN(C)CC2(F)F)nn2cc(F)c(-c3ccc4nnn(CC(F)F)c4n3)c12]

(R)—N²-(3,3-Difluoro-1-methylpiperidin-4-yl)-5-(3-(2,2-difluoroethyl)-3H-[1,2,3]triazolo[4,5-b]pyridin-5-yl)-6-fluoro-N⁴-methylpyrrolo[2,1-f][1,2,4]triazine-2,4-diamine 291.

Fluffy yellow solid (4 mg, 0.008 mmol, 38.9% yield). ¹H NMR (499 MHz, DMSO-d₆) δ ppm 1.69-1.88 (2H, m), 2.15 (1H, td, J=10.95, 2.46 Hz), 2.25 (3H, s), 2.35 (1H, dd, J=26.85, 11.77 Hz), 2.77 (1H, br d, J=12.05 Hz), 2.97-3.07 (1H, m), 3.10 (3H, d, J=4.93 Hz), 4.13-4.31 (1H, m), 5.27 (2H, td, J=16.08, 2.60 Hz), 6.47 (1H, d, J=9.31 Hz), 6.72 (2H, tt, J=54.30, 2.87 Hz), 7.67 (1H, d, J=2.19 Hz), 7.96 (1H, d, J=9.03 Hz), 8.67 (1H, d, J=8.76 Hz), 10.22 (1H, q, J=4.65 Hz); ESIMS found for C₂₀H₂₁F₅N₁₀ m/z 497.2 (M+1).

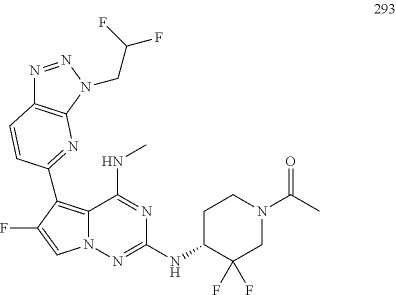 [SMILES: CNc1nc(N[C@@H]2CCN(C(C)=O)CC2(F)F)nn2cc(F)c(-c3ccc4nnn(CC(F)F)c4n3)c12]

(R)-1-(4-((5-(3-(2,2-Difluoroethyl)-3H-[1,2,3]triazolo[4,5-b]pyridin-5-yl)-6-fluoro-4-(methylamino)pyrrolo[2,1-f][1,2,4]triazin-2-yl)amino)-3,3-difluoropiperidin-1-yl)ethan-1-one 293.

Fluffy yellow solid (12 mg, 0.023 mmol, 96.4% yield). ¹H NMR (499 MHz, DMSO-d₆) δ ppm 1.54-1.81 (1H, m), 1.82-1.99 (1H, m), 2.07 (3H, s), 2.98 (1H, br t, J=11.50 Hz), 3.12 (3H, d, J=4.65 Hz), 3.60-3.91 (1H, m), 4.06-4.28 (1H, m), 4.40-4.60 (2H, m), 5.28 (2H, td, J=16.02, 2.46 Hz), 6.72 (1H, tt, J=54.00, 2.74 Hz), 6.67 (1H, br d, J=9.58 Hz), 7.66 (1H, s), 7.97 (1H, d, J=9.03 Hz), 8.69 (1H, d, J=9.03 Hz), 10.24 (1H, q, J=4.65 Hz); ESIMS found for C₂₁H₂₁F₅N₁₀O m/z 525.2 (M+1).

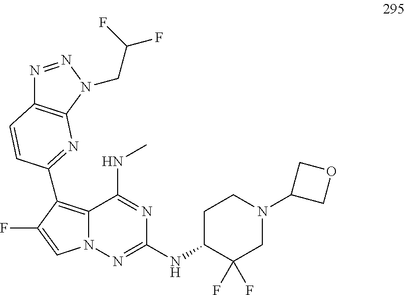 [SMILES: CNc1nc(N[C@@H]2CCN(C3COC3)CC2(F)F)nn2cc(F)c(-c3ccc4nnn(CC(F)F)c4n3)c12]

(R)—N²-(3,3-Difluoro-1-(oxetan-3-yl)piperidin-4-yl)-5-(3-(2,2-difluoroethyl)-3H-[1,2,3]triazolo[4,5-b]pyridin-5-yl)-6-fluoro-N⁴-methylpyrrolo[2,1-f][1,2,4]triazine-2,4-diamine 295.

Fluffy yellow solid (10 mg, 0.019 mmol, 64.0% yield). ¹H NMR (499 MHz, DMSO-d₆) δ ppm 1.73-1.83 (1H, m), 1.84-1.91 (1H, m), 2.09-2.18 (1H, m), 2.37 (1H, dd, J=26.60, 11.50 Hz), 2.75 (1H, br d, J=11.50 Hz), 2.93-3.06 (1H, m), 3.11 (3H, d, J=4.65 Hz), 3.60 (1H, quin, J=6.30 Hz), 4.19-4.36 (1H, m), 4.43 (2H, dt, J=14.92, 6.09 Hz), 4.55 (2H, td, J=6.64, 3.70 Hz), 5.27 (2H, td, J=16.08, 2.60 Hz), 6.55 (1H, d, J=9.31 Hz), 6.72 (1H, tt, J=54.00, 2.87 Hz), 7.66 (1H, d, J=2.19 Hz), 7.96 (1H, d, J=9.03 Hz), 8.68 (1H, d, J=8.76 Hz), 10.23 (1H, q, J=4.56 Hz); ESIMS found for C₂₂H₂₃F₅N₁₀O m/z 539.2 (M+1).

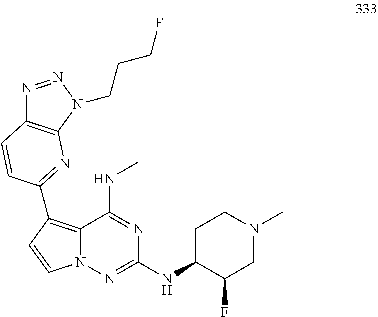 [SMILES: CNc1nc(N[C@H]2CCN(C)C[C@H]2F)nn2ccc(-c3ccc4nnn(CCCF)c4n3)c12]

N²-((3R,4S)-3-Fluoro-1-methylpiperidin-4-yl)-5-(3-(3-fluoropropyl)-3H-[1,2,3]triazolo[4,5-b]pyridin-5-yl)-N⁴-methylpyrrolo[2,1-f][1,2,4]triazine-2,4-diamine 333.

Light yellow solid (10 mg, 0.022 mmol, 57.0% yield). ¹H NMR (499 MHz, DMSO-d₆) δ ppm 1.62-1.73 (1H, m), 1.89 (1H, qd, J=12.27, 3.97 Hz), 2.05 (1H, br t, J=10.81 Hz), 2.17 (1H, dd, J=37.85, 12.32 Hz), 2.18 (3H, s), 2.43 (2H, dquin, J=27.15, 6.30, 6.30, 6.30, 6.30 Hz), 2.79 (1H, br d, J=11.23 Hz), 3.04 (1H, br t, J=10.81 Hz), 3.13 (3H, d, J=4.65 Hz), 3.65-3.86 (1H, m), 4.54 (2H, dt, J=47.20, 5.48 Hz), 4.82 (2H, t, J=6.98 Hz), 4.88 (1H, d, J=49.35 Hz), 6.08 (1H, d, J=7.94 Hz), 7.31 (1H, d, J=3.01 Hz), 7.43 (1H, d, J=3.01 Hz), 8.07 (1H, d, J=9.03 Hz), 8.51 (1H, d, J=9.04 Hz), 11.06 (1H, q, J=4.56 Hz); ESIMS found for C₂₁H₂₆F₂N₁₀ m/z 457.2 (M+1).

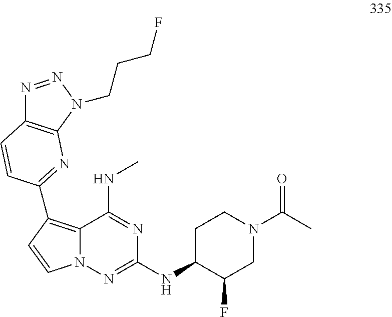 [SMILES: CNc1nc(N[C@H]2CCN(C(C)=O)C[C@H]2F)nn2ccc(-c3ccc4nnn(CCCF)c4n3)c12]

1-((3R,4S)-3-Fluoro-4-((5-(3-(3-fluoropropyl)-3H-[1,2,3]triazolo[4,5-b]pyridin-5-yl)-4-(methylamino)pyrrolo[2,1-f][1,2,4]triazin-2-yl)amino)piperidin-1-yl)ethan-1-one 335.

Light yellow solid (13 mg, 0.027 mmol, 69.8% yield). ¹H NMR (499 MHz, DMSO-d₆) δ ppm 1.60-1.90 (2H, m), 1.97-2.06 (3H, m), 2.61-2.77 (1H, m), 2.88 (1H, dd, J=39.50, 14.23 Hz), 3.14 (3H, d, J=4.65 Hz), 3.85-4.00 (1H, m), 4.02-4.19 (1H, m), 4.55 (2H, dt, J=47.20, 5.48 Hz), 4.63-4.76 (1H, m), 4.82 (2H, t, J=6.84 Hz), 4.96 (1H, d, J=49.35 Hz), 6.28 (1H, br d, J=7.94 Hz), 7.32 (1H, d, J=3.29 Hz), 7.43 (1H, d, J=3.01 Hz), 8.07 (1H, d, J=9.04 Hz), 8.52 (1H, d, J=9.03 Hz), 11.08 (1H, q, J=4.47 Hz); ESIMS found for C₂₂H₂₆F₂N₁₀O m/z 485.2 (M+1).

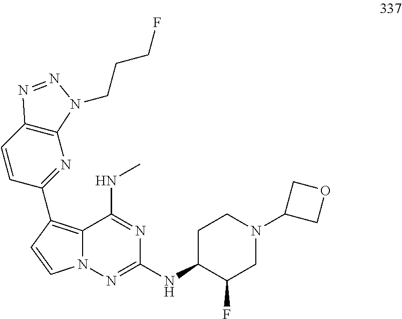 [SMILES: CNc1nc(N[C@H]2CCN(C3COC3)C[C@H]2F)nn2ccc(-c3ccc4nnn(CCCF)c4n3)c12]

N²-((3R,4S)-3-Fluoro-1-(oxetan-3-yl)piperidin-4-yl)-5-(3-(3-fluoropropyl)-3H-[1,2,3]triazolo[4,5-b]pyridin-5-yl)-N⁴-methylpyrrolo[2,1-f][1,2,4]triazine-2,4-diamine 337.

Light yellow solid (7 mg, 0.014 mmol, 36.5% yield). ¹H NMR (499 MHz, DMSO-d₆) δ ppm 1.66-1.77 (1H, m), 1.88 (1H, qd, J=12.00, 3.42 Hz), 1.96-2.06 (1H, m), 2.13 (1H, dd, J=37.30, 12.87 Hz), 2.43 (2H, dquin, J=27.15, 6.18, 6.18, 6.18, 6.18 Hz), 2.75 (1H, br d, J=10.40 Hz), 2.91-3.04 (1H, m), 3.14 (3H, d, J=4.65 Hz), 3.49 (1H, quin, J=6.30 Hz), 3.74-3.90 (1H, m), 4.40 (1H, t, J=6.16 Hz), 4.46 (1H, t, J=6.16 Hz), 4.54 (2H, dt, J=47.15, 5.48 Hz), 4.54 (2H, td, J=6.50, 3.15 Hz), 4.82 (2H, t, J=6.84 Hz), 4.90 (1H, d, J=49.35 Hz), 6.15 (1H, d, J=7.94 Hz), 7.32 (1H, d, J=3.01 Hz), 7.43 (1H, d, J=3.01 Hz), 8.07 (1H, d, J=9.03 Hz), 8.51 (1H, d, J=9.04 Hz), 11.07 (1H, q, J=4.38 Hz); ESIMS found for C₂₃H₂₈F₂N₁₀O m/z 499.3 (M+1).

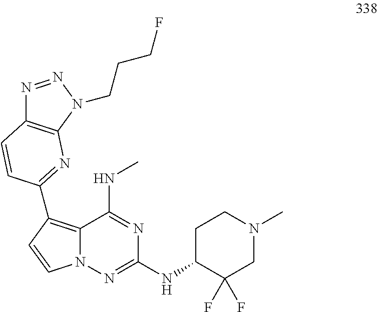 [SMILES: CNc1nc(N[C@@H]2CCN(C)CC2(F)F)nn2ccc(-c3ccc4nnn(CCCF)c4n3)c12]

(R)—N²-(3,3-Difluoro-1-methylpiperidin-4-yl)-5-(3-(3-fluoropropyl)-3H-[1,2,3]triazolo[4,5-b]pyridin-5-yl)-N⁴-methylpyrrolo[2,1-f][1,2,4]triazine-2,4-diamine 338.

Light yellow solid (23 mg, 0.049 mmol, 69.8% yield). ¹H NMR (499 MHz, DMSO-d₆) δ ppm 1.72-1.88 (2H, m), 2.10-2.20 (1H, m), 2.25 (3H, s), 2.36 (1H, dd, J=27.80, 11.70 Hz), 2.43 (2H, dquin, J=27.15, 6.00, 6.00, 6.00, 6.00 Hz), 2.77 (1H, br d, J=11.77 Hz), 2.97-3.10 (1H, m), 3.15 (3H, d, J=4.65 Hz), 4.17-4.34 (1H, m), 4.54 (2H, dt, J=47.20, 5.61 Hz), 4.83 (2H, t, J=6.98 Hz), 6.32 (1H, d, J=9.31 Hz), 7.33 (1H, d, J=3.01 Hz), 7.44 (1H, d, J=3.01 Hz), 8.07 (1H, d, J=9.03 Hz), 8.52 (1H, d, J=8.76 Hz), 11.09 (1H, q, J=4.47 Hz); ESIMS found for C₂₁H₂₅F₃N₁₀ m/z 475.2 (M+1).

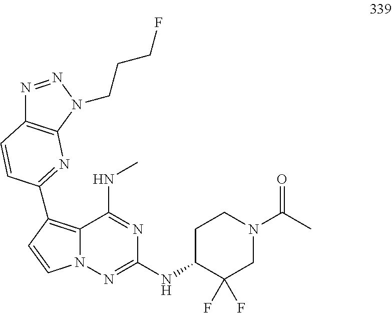 [SMILES: CNc1nc(N[C@@H]2CCN(C(C)=O)CC2(F)F)nn2ccc(-c3ccc4nnn(CCCF)c4n3)c12]

(R)-1-(3,3-Difluoro-4-((5-(3-(3-fluoropropyl)-3H-[1,2,3]triazolo[4,5-b]pyridin-5-yl)-4-(methylamino)pyrrolo[2,1-f][1,2,4]triazin-2-yl)amino)piperidin-1-yl)ethan-1-one 339.

Light yellow solid (28 mg, 0.056 mmol, 80.2% yield). ¹H NMR (499 MHz, DMSO-d₆) δ ppm 1.55-1.81 (1H, m), 1.82-1.96 (1H, m), 2.07 (3H, s), 2.43 (2H, dquin, J=27.15, 5.75, 5.75, 5.75, 5.75 Hz), 2.99 (1H, br t, J=11.09 Hz), 3.16 (3H, d, J=4.65 Hz), 3.60-3.90 (1H, m), 4.08-4.27 (1H, m), 4.43-4.57 (2H, m), 4.55 (2H, dt, J=47.15, 5.61 Hz), 4.83 (2H, t, J=6.98 Hz), 6.54 (1H, d, J=4.38 Hz), 7.34 (1H, d, J=3.01 Hz), 7.43 (1H, d, J=2.74 Hz), 8.08 (1H, d, J=9.31 Hz), 8.52 (1H, d, J=9.03 Hz), 11.11 (1H, q, J=4.47 Hz); ESIMS found for C₂₂H₂₅F₃N₁₀O m/z 503.2 (M+1).

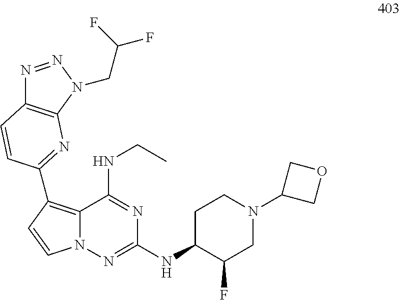 [SMILES: CCNc1nc(N[C@H]2CCN(C3COC3)C[C@H]2F)nn2ccc(-c3ccc4nnn(CC(F)F)c4n3)c12]

5-(3-(2,2-Difluoroethyl)-3H-[1,2,3]triazolo[4,5-b]pyridin-5-yl)-N⁴-ethyl-N²-((3R,4S)-3-fluoro-1-(oxetan-3-yl)piperidin-4-yl)pyrrolo[2,1-f][1,2,4]triazine-2,4-diamine 403.

Fluffy yellow solid (2 mg, 0.004 mmol, 7.8% yield). ¹H NMR (499 MHz, DMSO-d₆) δ ppm 1.25 (3H, t, J=7.12 Hz), 1.68-1.75 (1H, m), 1.88 (1H, qd, J=12.05, 3.56 Hz), 1.96-2.04 (1H, m), 2.13 (1H, dd, J=37.30, 12.59 Hz), 2.75 (1H, br d, J=9.58 Hz), 2.94-3.05 (1H, m), 3.49 (1H, quin, J=6.43 Hz), 3.67 (2H, quin, J=6.84 Hz), 3.75-3.90 (1H, m), 4.40 (1H, t, J=6.16 Hz), 4.46 (1H, t, J=6.16 Hz), 4.54 (2H, td, J=6.57, 3.01 Hz), 4.90 (1H, d, J=49.65 Hz), 5.21 (2H, td, J=15.74, 2.74 Hz), 6.16 (1H, d, J=8.21 Hz), 6.72 (1H, tt, J=54.05, 3.15 Hz), 7.33 (1H, d, J=3.01 Hz), 7.44 (1H, d, J=3.01 Hz), 8.11 (1H, d, J=9.31 Hz), 8.56 (1H, d, J=8.76 Hz), 10.89 (1H, t, J=5.89 Hz); ESIMS found for C₂₃H₂₇F₃N₁₀O m/z 517.2 (M+1).

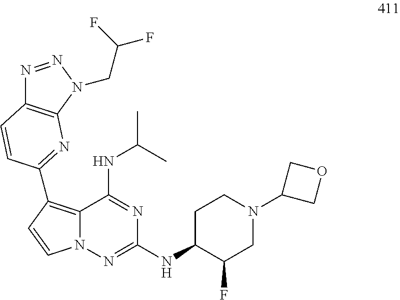 [SMILES: CC(C)Nc1nc(N[C@H]2CCN(C3COC3)C[C@H]2F)nn2ccc(-c3ccc4nnn(CC(F)F)c4n3)c12]

5-(3-(2,2-Difluoroethyl)-3H-[1,2,3]triazolo[4,5-b]pyridin-5-yl)-N²-((3R,4S)-3-fluoro-1-(oxetan-3-yl)piperidin-4-yl)-N⁴-isopropylpyrrolo[2,1-f][1,2,4]triazine-2,4-diamine 411.

Fluffy yellow solid (2 mg, 0.004 mmol, 7.6% yield). ¹H NMR (499 MHz, DMSO-d₆) δ ppm 1.34 (3H, d, J=3.01 Hz), 1.35 (3H, d, J=3.01 Hz), 1.71 (1H, br dd, J=13.14, 3.29 Hz), 1.88 (1H, qd, J=12.09, 3.42 Hz), 1.95-2.05 (1H, m), 2.13 (1H, dd, J=37.30, 12.59 Hz), 2.71-2.81 (1H, m), 2.93-3.05 (1H, m), 3.49 (1H, quin, J=6.30 Hz), 3.71-3.90 (1H, m), 4.40 (1H, t, J=6.16 Hz), 4.46 (1H, t, J=6.16 Hz), 4.54 (2H, td, J=6.43, 3.01 Hz), 4.61 (1H, dq, J=14.10, 6.80 Hz), 4.89 (1H, d, J=49.65 Hz), 5.21 (2H, td, J=15.06, 3.29 Hz), 6.18 (1H, d, J=8.21 Hz), 6.73 (1H, tt, J=54.30, 3.42 Hz), 7.23 (1H, d, J=3.01 Hz), 7.46 (1H, d, J=3.01 Hz), 8.07 (1H, d, J=9.03 Hz), 8.57 (1H, d, J=9.03 Hz), 9.62 (1H, d, J=8.21 Hz); ESIMS found for C₂₄H₂₉F₃N₁₀O m/z 531.3 (M+1).

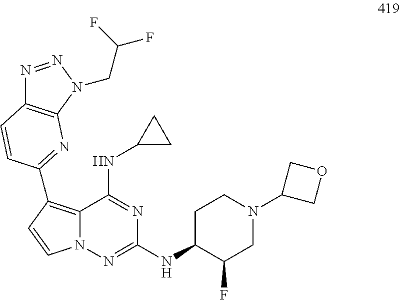 [SMILES: FC(F)Cn1nnc2ccc(-c3ccn4nc(N[C@H]5CCN(C6COC6)C[C@H]5F)nc(NC5CC5)c34)nc21]

N⁴-Cyclopropyl-5-(3-(2,2-difluoroethyl)-3H-[1,2,3]triazolo[4,5-b]pyridin-5-yl)-N²-((3R,4S)-3-fluoro-1-(oxetan-3-yl)piperidin-4-yl)pyrrolo[2,1-f][1,2,4]triazine-2,4-diamine 419.

Fluffy yellow solid (2 mg, 0.004 mmol, 7.6% yield). ¹H NMR (499 MHz, DMSO-d₆) δ ppm 0.69-0.78 (2H, m), 0.82-0.93 (2H, m), 1.68-1.77 (1H, m), 1.90 (1H, qd, J=12.14, 3.56 Hz), 1.96-2.03 (1H, m), 2.13 (1H, dd, J=37.60, 12.59 Hz), 2.73-2.80 (1H, m), 2.80-2.87 (1H, m), 2.94-3.05 (1H, m), 3.49 (1H, quin, J=6.50 Hz), 3.76-3.94 (1H, m), 4.40 (1H, t, J=6.16 Hz), 4.46 (1H, t, J=6.16 Hz), 4.54 (2H, td, J=6.43, 3.29 Hz), 4.94 (1H, d, J=49.90 Hz), 5.23 (2H, td, J=15.40, 3.15 Hz), 6.19 (1H, br d, J=7.94 Hz), 6.73 (1H, tt, J=54.30, 3.29 Hz), 7.34 (1H, d, J=3.01 Hz), 7.46 (1H, d, J=2.74 Hz), 8.11 (1H, d, J=9.03 Hz), 8.55 (1H, d, J=8.76 Hz), 10.96 (1H, s); ESIMS found for C₂₄H₂₇F₃N₁₀O m/z 529.2 (M+1).

### Example 6

Representative compounds were screened using the assay procedure for DYRK1A kinase activity as described below.

Each compound was dissolved in DMSO as a 10 mM stock and used to prepare compound source plates. Serial dilution (1:3, 11-point dose-response curves from 10 μM to 0.00016 μM) and compound transfer was performed using the ECHO 550 (Labcyte, Sunnyvale, CA) into 1536-well black-walled round bottom plates (Corning).

The DYRK1A kinase assay was run using the Ser/Thr 18 peptide Z-lyte assay kit according to manufacturer's instructions (Life Technologies—a Division of Thermo-Fisher). This is a non-radioactive assay using fluorescence resonance energy transfer (FRET) between coumarin and fluorescein to detect kinase activity which is represented as a ratio of coumarin emission/fluorescein emission.

Briefly, recombinant DYRK1A kinase, ATP and Ser/Thr peptide 18 were prepared in 1× Kinase buffer to final concentrations of 0.25 μg/mL, 15 μM, and 4 μM respectively. The mixture was allowed to incubate with the representative compounds for one hour at room temperature. All reactions were performed in duplicate. Unphosphorylated (“0% Control”) and phosphorylated (“100% control”) forms of Ser/Thr 18 served as control reactions. Additionally, an 11-point dose-response curve of Staurosporine (1 uM top) was run to serve as a positive compound control.

After incubation, Development Reagent A was diluted in Development Buffer then added to the reaction and allowed to further incubate for one hour at room temperature. The plate was read at Ex 400 Em 455 to detect the coumarin signal and Ex 400 Em 520 to measure the signal (EnVision Multilabel Plate Reader, PerkinElmer).

The Emission ratio (Em) was calculated as a ratio of the coumarin (C) emission signal (at 445 nm)/Fluorescein (F) emission signal (at 520 nm). The percent phosphorylation was then calculated using the following formula: [1−((Em ratio×F100%)−C100%)/((C0%−C100%)+(Em ratio×(F100%−F0%)))]. Dose-response curves were generated, and inhibitory concentration (IC₅₀) values were calculated using non-linear regression curve fit in the Dotmatics' Studies Software (Bishops Stortford, UK).

Table 3 shows the measured activity for representative compounds of Formula I as described herein.

TABLE 3

| Compound | IC₅₀ (μM) |
|---|---|
| 38 | 0.0034 |
| 39 | 0.0031 |
| 40 | 0.0027 |
| 41 | 0.0020 |
| 43 | 0.0031 |
| 46 | 0.0030 |
| 48 | 0.0022 |
| 53 | 0.0025 |
| 54 | 0.0024 |
| 55 | 0.0040 |
| 56 | 0.0014 |
| 58 | 0.0019 |
| 112 | 0.0012 |
| 113 | 0.0008 |
| 114 | 0.0016 |
| 115 | 0.0010 |
| 116 | 0.0016 |
| 117 | 0.0009 |
| 118 | 0.0005 |
| 119 | 0.0014 |
| 120 | 0.0007 |
| 121 | 0.0008 |
| 122 | 0.0011 |
| 123 | 0.0019 |
| 124 | 0.0010 |
| 125 | 0.0017 |
| 126 | 0.0013 |
| 127 | 0.0015 |
| 128 | 0.0014 |
| 129 | 0.0014 |
| 131 | 0.0047 |
| 132 | 0.0012 |
| 134 | 0.0050 |
| 135 | 0.0064 |
| 139 | 0.0012 |
| 141 | 0.0034 |
| 142 | 0.0027 |
| 143 | 0.0013 |
| 144 | 0.0042 |
| 145 | 0.0017 |
| 146 | 0.0008 |
| 147 | 0.0014 |
| 149 | 0.0008 |
| 157 | 0.0158 |
| 160 | 0.0022 |
| 162 | 0.0031 |
| 163 | 0.0013 |
| 164 | 0.0018 |
| 165 | 0.0023 |
| 166 | 0.0029 |
| 168 | 0.0014 |
| 174 | 0.0020 |
| 175 | 0.0013 |
| 177 | 0.0015 |
| 214 | 0.0049 |
| 215 | 0.0023 |
| 216 | 0.0026 |
| 220 | 0.0052 |
| 221 | 0.0028 |
| 222 | 0.0032 |
| 233 | 0.0081 |
| 234 | 0.0008 |
| 235 | 0.0020 |
| 236 | 0.0021 |
| 237 | 0.0007 |
| 238 | 0.0014 |
| 239 | 0.0006 |
| 248 | 0.002 |
| 249 | 0.0019 |
| 250 | 0.0007 |
| 251 | 0.0022 |
| 252 | 0.0019 |
| 254 | 0.0022 |
| 256 | 0.0035 |
| 257 | 0.0014 |
| 258 | 0.0013 |
| 260 | 0.0018 |
| 261 | 0.0006 |
| 262 | 0.0013 |
| 264 | 0.0012 |
| 266 | 0.0007 |
| 267 | 0.0005 |
| 270 | 0.0017 |
| 271 | 0.0029 |
| 272 | 0.0050 |
| 273 | 0.0027 |
| 274 | 0.0036 |
| 275 | 0.0084 |
| 276 | 0.0012 |
| 286 | 0.0036 |
| 288 | 0.0024 |
| 290 | 0.0012 |
| 291 | 0.0012 |
| 293 | 0.0019 |
| 295 | 0.0014 |
| 333 | 0.0112 |
| 335 | 0.0034 |
| 337 | 0.0044 |
| 338 | 0.0031 |
| 339 | 0.0035 |
| 403 | 0.0042 |
| 411 | 0.0097 |
| 419 | 0.0082 |

What is claimed is:

1. A compound, or a pharmaceutically acceptable salt thereof, of Formula I: 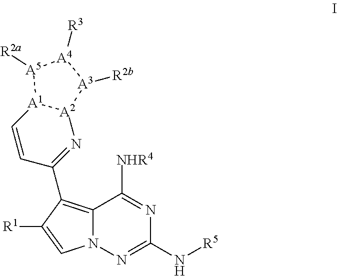
wherein:
A¹, A², A³, A⁴, and A⁵ are independently selected from the group consisting of carbon and nitrogen with the proviso that at least two of A¹, A², A³, A⁴, and A⁵ are nitrogen;
if A¹ and A³ are both nitrogen then R^{2b} is absent; if A¹ and A⁴ are both nitrogen then R³ is absent; if A¹ and A⁵ are both nitrogen then R^{2a} is absent; if A¹, A³, and A⁴ are all nitrogen then R^{2b} and R³ are absent; if A¹, A⁴, and A⁵ are all nitrogen then R^{2a} and R³ are absent; if A¹, A³, and A⁵ are all nitrogen then R^{2a} and R^{2b} are absent; if A¹, A³, A⁴, and A⁵ are all nitrogen then R^{2a}, R^{2b}, and R³ are all absent; if A² and A³ are both nitrogen then R^{2b} is absent; if A² and A⁴ are both nitrogen then R³ is absent; if A² and A⁵ are both nitrogen then R^{2a} is absent; if A², A³, and A⁴ are all nitrogen then R^{2b} and R³ are absent; if A², A⁴, and A⁵ are all nitrogen then R^{2a} and R³ are absent; if A², A³, and A⁵ are all nitrogen then R^{2a} and R^{2b} are absent; if A², A³, A⁴, and A⁵ are all nitrogen then R^{2a}, R^{2b}, and R³ are all absent;
R¹ is selected from the group consisting of H and halide;
R^{2a} is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R^{2b} is selected from the group consisting of H, unsubstituted —(C₁₋₉ alkyl), unsubstituted —(C₂₋₉ alkenyl), unsubstituted —(C₂₋₉ alkynyl), unsubstituted —(C₁₋₉ haloalkyl), and —C(═O)N(R⁶)₂;
R³ is selected from the group consisting of H, unsubstituted —(C₁₋₉ alkyl), unsubstituted —(C₂₋₉ alkenyl), unsubstituted —(C₂₋₉ alkynyl), and unsubstituted —(C₁₋₉ haloalkyl);
R⁴ is selected from the group consisting of H, unsubstituted —(C₁₋₆ alkyl), unsubstituted —(C₂₋₆ alkenyl), unsubstituted —(C₂₋₆ alkynyl), unsubstituted —(C₁₋₆ haloalkyl), and -carbocyclyl optionally substituted with 1-10 R⁷;
R⁵ is selected from the group consisting of -heterocyclyl optionally substituted with 1-10 R⁸ and -carbocyclyl optionally substituted with 1-12 R⁹;
each R⁶ is independently selected from the group consisting of H, unsubstituted —(C₁₋₉ alkyl), unsubstituted —(C₂₋₉ alkenyl), and unsubstituted —(C₂₋₉ alkynyl);
each R⁷ is halide;
each R⁸ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), -heterocyclyl optionally substituted with 1-10 R¹⁰, —(C₁₋₅ alkylene)OR¹¹, —SO₂R¹², and —C(═O)R¹³ wherein the —(C₁₋₅ alkylene) is optionally substituted with 1-5 halide and/or 1-3 unsubstituted —(C₁₋₃ alkyl);
each R⁹ is independently selected from the group consisting of halide, unsubstituted —(C₁₋₉ alkyl), unsubstituted —(C₂₋₉ alkenyl), unsubstituted —(C₂₋₉ alkynyl), unsubstituted —(C₁₋₉ haloalkyl), —OR¹⁴, and —NR¹⁵C(═O)R¹⁶;
each R¹⁰ is halide;
R¹¹ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R¹² is selected from the group consisting of unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R¹³ is selected from the group consisting of unsubstituted —(C₁₋₆ alkyl), unsubstituted —(C₂₋₆ alkenyl), unsubstituted —(C₂₋₆ alkynyl), unsubstituted —(C₁₋₆ haloalkyl), and —(C₁₋₅ alkylene)OR¹⁸, wherein the —(C₁₋₅ alkylene) is optionally substituted with 1-5 halide and/or 1-3 unsubstituted —(C₁₋₃ alkyl);
R¹⁴ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl), and —(C₁₋₅ alkylene)OR¹⁸, wherein the —(C₁₋₅ alkylene) is optionally substituted with 1-5 halide and/or 1-3 unsubstituted —(C₁₋₃ alkyl);
R¹⁵ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R¹⁶ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R¹⁷ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl);
R¹⁸ is selected from the group consisting of H, unsubstituted —(C₁₋₆ alkyl), unsubstituted —(C₂₋₆ alkenyl), unsubstituted —(C₂₋₆ alkynyl), and unsubstituted —(C₁₋₆ haloalkyl); and
wherein each H atom is optionally, independently replaced by ²H (D) (deuterium).

2. The compound of claim 1, wherein R¹ is H.

3. The compound of claim 1, wherein R¹ is F.

4. The compound of claim 1, wherein A¹ and A⁵ are nitrogen, and A², A³, and A⁴ are carbon.

5. The compound of claim 1, wherein A² and A⁵ are nitrogen, and A¹, A³, and A⁴ are carbon.

6. The compound of claim 1, wherein A³ and A⁵ are nitrogen, and A¹, A², and A⁴ are carbon.

7. The compound of claim 1, wherein A³, A⁴, and A⁵ are nitrogen, and A¹ and A² are carbon.

8. The compound of claim 1, wherein R^{2b} is selected from the group consisting of H, unsubstituted —(C₁₋₃haloalkyl), and —C(═O)NHMe; and R³ is H.

9. The compound of claim 1, wherein R^{2b} is selected from the group consisting of unsubstituted —(C₁₋₃ alkyl) and unsubstituted —(C₁₋₃ haloalkyl); and R³ is selected from the group consisting of H and Me.

10. The compound of claim 1, wherein R^{2b} is selected from the group consisting of unsubstituted —(C₁₋₃ alkyl) and unsubstituted —(C₁₋₃ haloalkyl).

11. The compound of claim 1, wherein R⁴ is H.

12. The compound of claim 1, wherein R⁴ is Me.

13. The compound of claim 1, wherein R⁵ is selected from the group consisting of -heterocyclyl optionally substituted with 1-4 R⁸ and -carbocyclyl optionally substituted with 1-4 R⁹.

14. The compound of claim 1, wherein each R⁸ is selected from the group consisting of Me, —C(═O)Me, —C(═O)CH₂OH, —SO₂Me, and

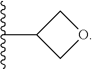 [SMILES: *C1COC1]

15. The compound of claim 1, wherein each R⁹ is independently selected from the group consisting of F, Me, —OH, —OMe, and —OCH₂CH₂OH.

16. The compound of claim 1, wherein the compound of Formula I is selected from the group consisting of: 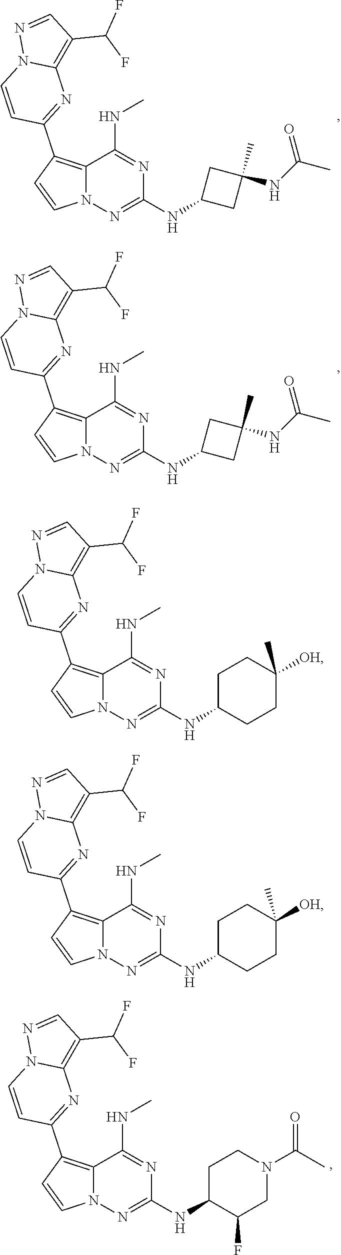 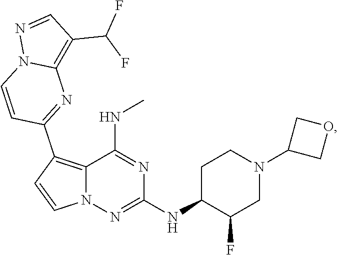 [SMILES: CNc1nc(N[C@H]2CCN(C3COC3)C[C@H]2F)nn2ccc(-c3ccn4ncc(C(F)F)c4n3)c12] 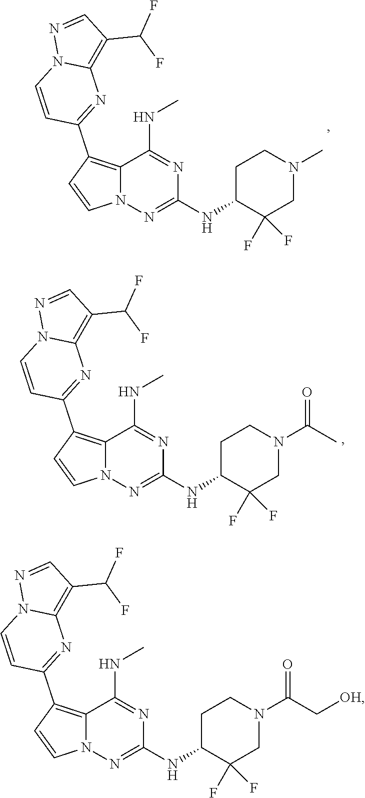 [SMILES: CNc1nc(N[C@@H]2CCN(C)CC2(F)F)nn2ccc(-c3ccn4ncc(C(F)F)c4n3)c12 ; CNc1nc(N[C@@H]2CCN(C(C)=O)CC2(F)F)nn2ccc(-c3ccn4ncc(C(F)F)c4n3)c12 ; CNc1nc(N[C@@H]2CCN(C(=O)CO)CC2(F)F)nn2ccc(-c3ccn4ncc(C(F)F)c4n3)c12] 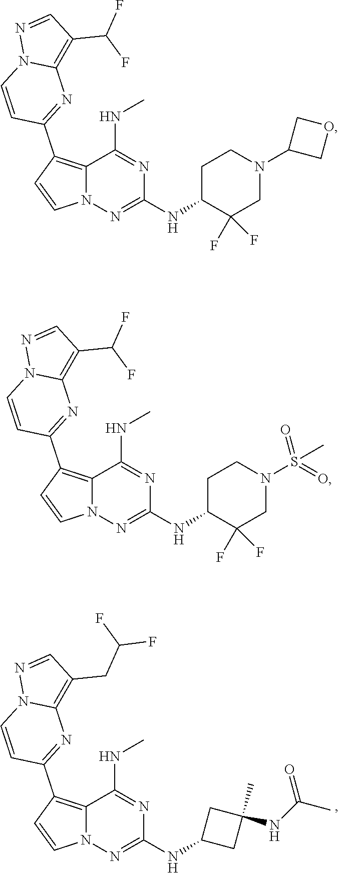 [SMILES: CNc1nc(N[C@@H]2CCN(C3COC3)CC2(F)F)nn2ccc(-c3ccn4ncc(C(F)F)c4n3)c12 ; CNc1nc(N[C@@H]2CCN(S(C)(=O)=O)CC2(F)F)nn2ccc(-c3ccn4ncc(C(F)F)c4n3)c12 ; CNc1nc(N[C@H]2C[C@](C)(NC(C)=O)C2)nn2ccc(-c3ccn4ncc(CC(F)F)c4n3)c12] 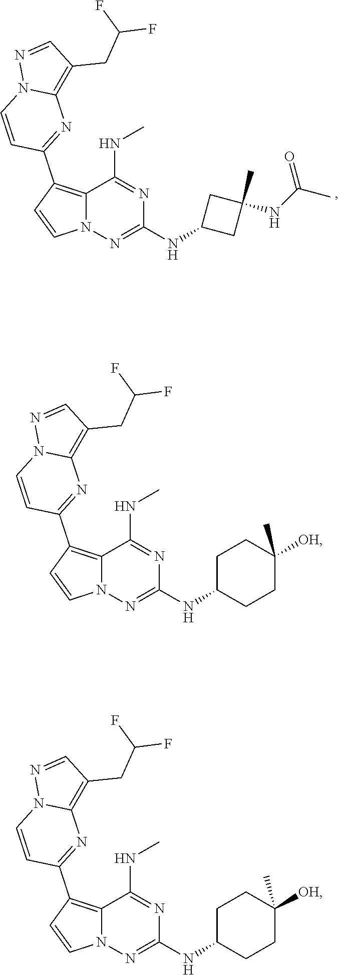 [SMILES: CNc1nc(N[C@H]2C[C@@](C)(NC(C)=O)C2)nn2ccc(-c3ccn4ncc(CC(F)F)c4n3)c12 ; CNc1nc(N[C@H]2CC[C@@](C)(O)CC2)nn2ccc(-c3ccn4ncc(CC(F)F)c4n3)c12 ; CNc1nc(N[C@H]2CC[C@](C)(O)CC2)nn2ccc(-c3ccn4ncc(CC(F)F)c4n3)c12] 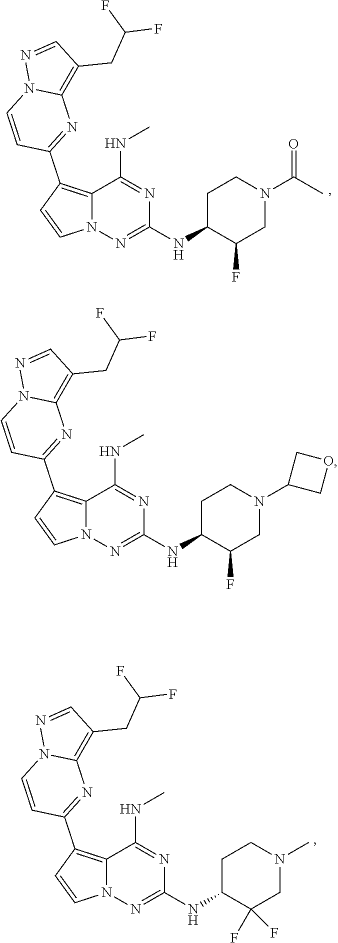 [SMILES: CNc1nc(N[C@H]2CCN(C(C)=O)C[C@H]2F)nn2ccc(-c3ccn4ncc(CC(F)F)c4n3)c12 ; CNc1nc(N[C@H]2CCN(C3COC3)C[C@H]2F)nn2ccc(-c3ccn4ncc(CC(F)F)c4n3)c12 ; CNc1nc(N[C@@H]2CCN(C)CC2(F)F)nn2ccc(-c3ccn4ncc(CC(F)F)c4n3)c12] 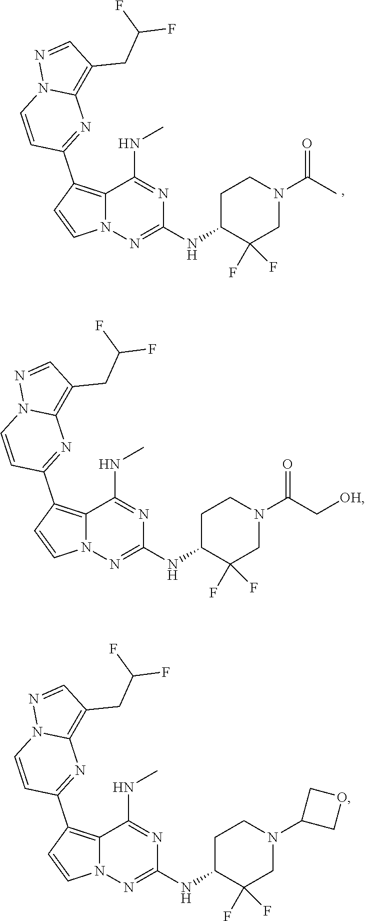 [SMILES: CNc1nc(N[C@@H]2CCN(C(C)=O)CC2(F)F)nn2ccc(-c3ccn4ncc(CC(F)F)c4n3)c12 ; CNc1nc(N[C@@H]2CCN(C(=O)CO)CC2(F)F)nn2ccc(-c3ccn4ncc(CC(F)F)c4n3)c12 ; CNc1nc(N[C@@H]2CCN(C3COC3)CC2(F)F)nn2ccc(-c3ccn4ncc(CC(F)F)c4n3)c12] 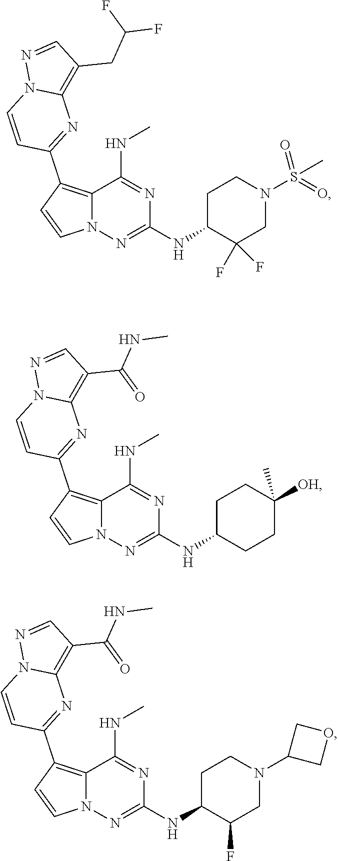 [SMILES: CNc1nc(N[C@@H]2CCN(S(C)(=O)=O)CC2(F)F)nn2ccc(-c3ccn4ncc(CC(F)F)c4n3)c12 ; CNC(=O)c1cnn2ccc(-c3ccn4nc(N[C@H]5CC[C@](C)(O)CC5)nc(NC)c34)nc12 ; CNC(=O)c1cnn2ccc(-c3ccn4nc(N[C@H]5CCN(C6COC6)C[C@H]5F)nc(NC)c34)nc12] 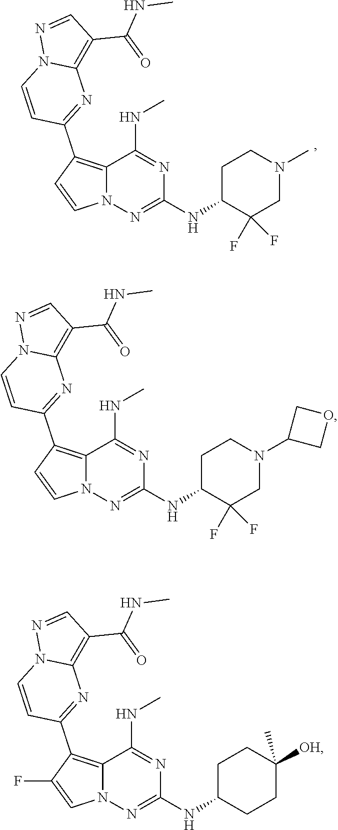 [SMILES: CNC(=O)c1cnn2ccc(-c3ccn4nc(N[C@@H]5CCN(C)CC5(F)F)nc(NC)c34)nc12 ; CNC(=O)c1cnn2ccc(-c3ccn4nc(N[C@@H]5CCN(C6COC6)CC5(F)F)nc(NC)c34)nc12 ; CNC(=O)c1cnn2ccc(-c3c(F)cn4nc(N[C@H]5CC[C@](C)(O)CC5)nc(NC)c34)nc12] 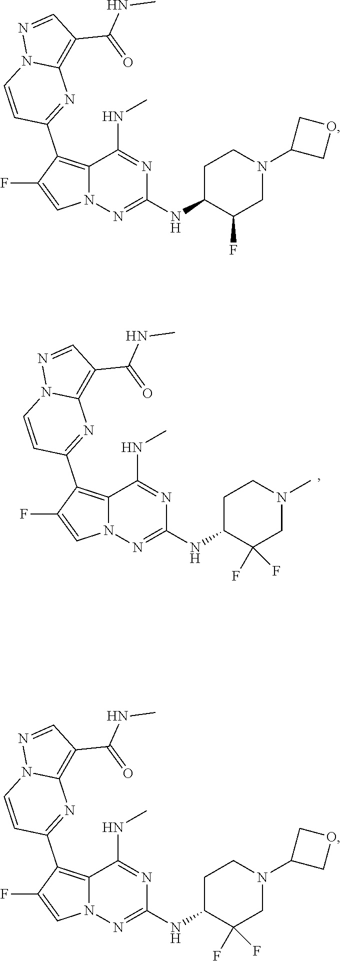 [SMILES: CNC(=O)c1cnn2ccc(-c3c(F)cn4nc(N[C@H]5CCN(C6COC6)C[C@H]5F)nc(NC)c34)nc12 ; CNC(=O)c1cnn2ccc(-c3c(F)cn4nc(N[C@@H]5CCN(C)CC5(F)F)nc(NC)c34)nc12 ; CNC(=O)c1cnn2ccc(-c3c(F)cn4nc(N[C@@H]5CCN(C6COC6)CC5(F)F)nc(NC)c34)nc12] 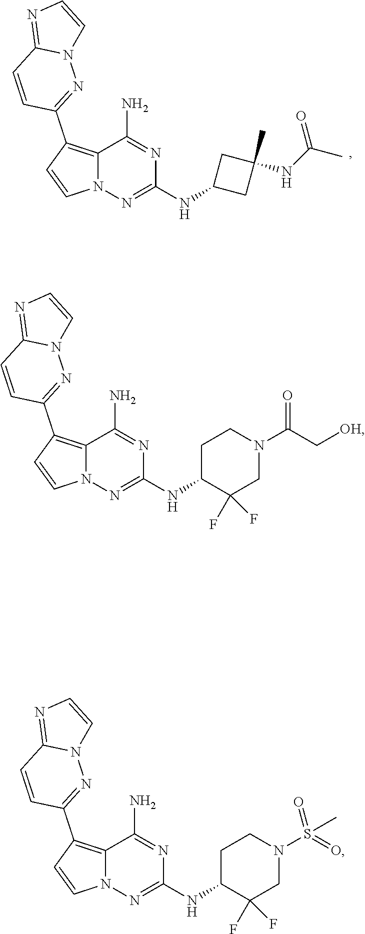 [SMILES: CC(=O)N[C@]1(C)C[C@H](Nc2nc(N)c3c(-c4ccc5nccn5n4)ccn3n2)C1 ; Nc1nc(N[C@@H]2CCN(C(=O)CO)CC2(F)F)nn2ccc(-c3ccc4nccn4n3)c12 ; CS(=O)(=O)N1CC[C@@H](Nc2nc(N)c3c(-c4ccc5nccn5n4)ccn3n2)C(F)(F)C1] 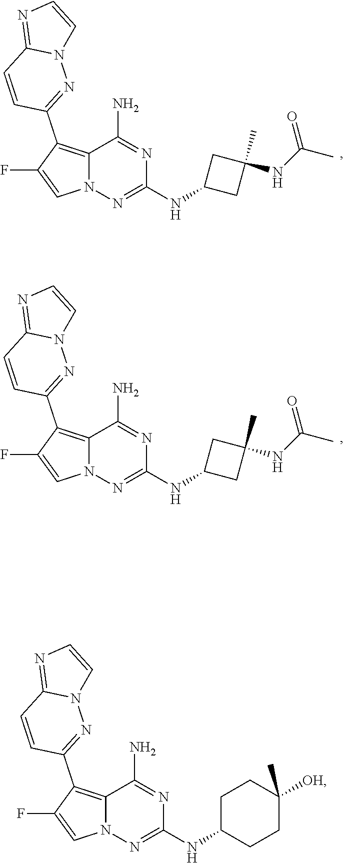 [SMILES: CC(=O)N[C@]1(C)C[C@@H](Nc2nc(N)c3c(-c4ccc5nccn5n4)c(F)cn3n2)C1 ; CC(=O)N[C@]1(C)C[C@H](Nc2nc(N)c3c(-c4ccc5nccn5n4)c(F)cn3n2)C1 ; C[C@]1(O)CC[C@@H](Nc2nc(N)c3c(-c4ccc5nccn5n4)c(F)cn3n2)CC1] 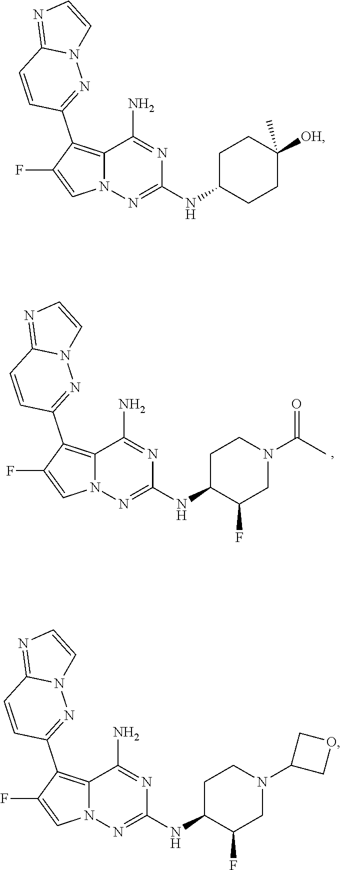 [SMILES: C[C@]1(O)CC[C@H](Nc2nc(N)c3c(-c4ccc5nccn5n4)c(F)cn3n2)CC1 ; CC(=O)N1CC[C@H](Nc2nc(N)c3c(-c4ccc5nccn5n4)c(F)cn3n2)[C@H](F)C1 ; Nc1nc(N[C@H]2CCN(C3COC3)C[C@H]2F)nn2cc(F)c(-c3ccc4nccn4n3)c12] 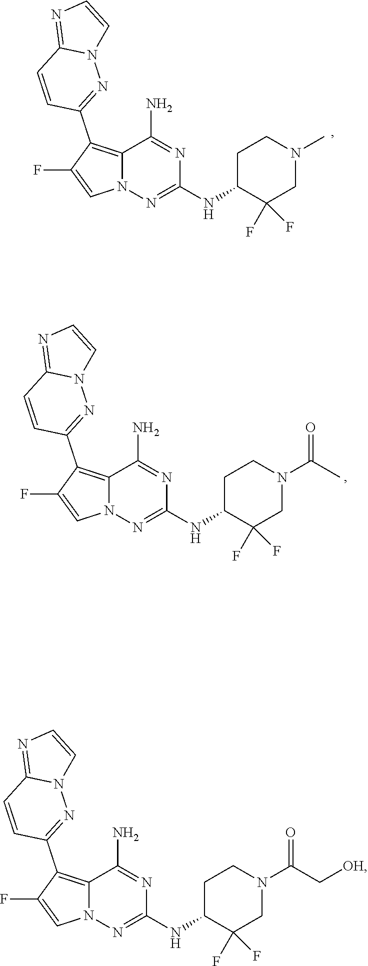 [SMILES: CN1CC[C@@H](Nc2nc(N)c3c(-c4ccc5nccn5n4)c(F)cn3n2)C(F)(F)C1 ; CC(=O)N1CC[C@@H](Nc2nc(N)c3c(-c4ccc5nccn5n4)c(F)cn3n2)C(F)(F)C1 ; Nc1nc(N[C@@H]2CCN(C(=O)CO)CC2(F)F)nn2cc(F)c(-c3ccc4nccn4n3)c12] 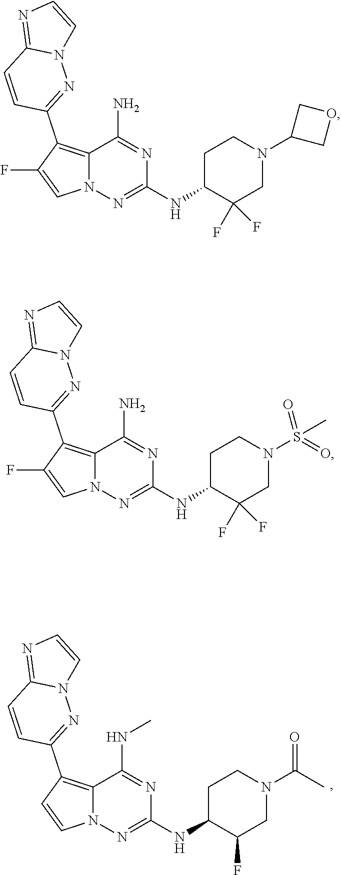 [SMILES: Nc1nc(N[C@@H]2CCN(C3COC3)CC2(F)F)nn2cc(F)c(-c3ccc4nccn4n3)c12 ; CS(=O)(=O)N1CC[C@@H](Nc2nc(N)c3c(-c4ccc5nccn5n4)c(F)cn3n2)C(F)(F)C1 ; CNc1nc(N[C@H]2CCN(C(C)=O)C[C@H]2F)nn2ccc(-c3ccc4nccn4n3)c12] 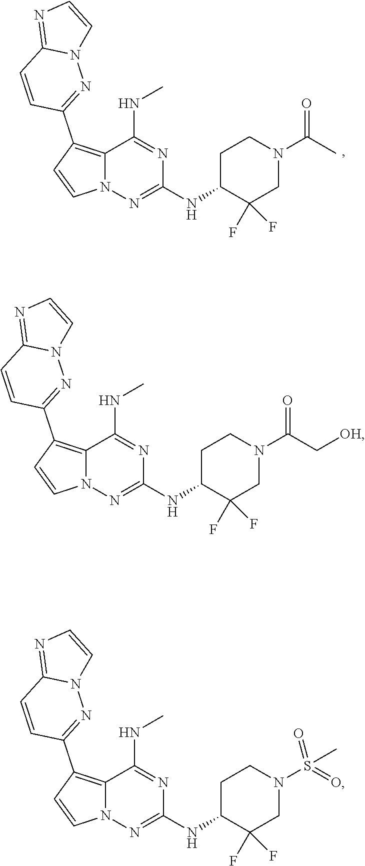 [SMILES: CNc1nc(N[C@@H]2CCN(C(C)=O)CC2(F)F)nn2ccc(-c3ccc4nccn4n3)c12 ; CNc1nc(N[C@@H]2CCN(C(=O)CO)CC2(F)F)nn2ccc(-c3ccc4nccn4n3)c12 ; CNc1nc(N[C@@H]2CCN(S(C)(=O)=O)CC2(F)F)nn2ccc(-c3ccc4nccn4n3)c12] 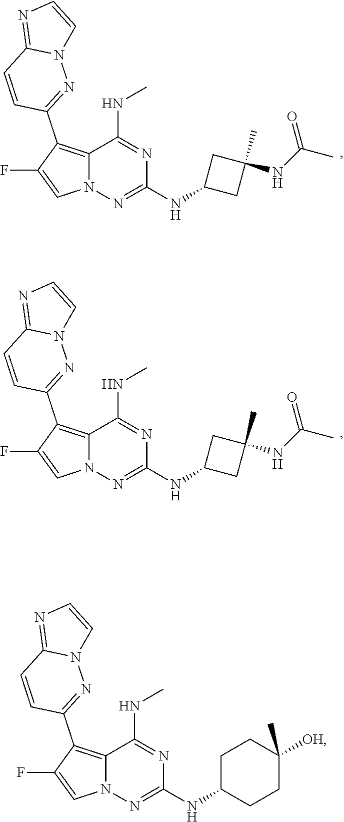 [SMILES: CNc1nc(N[C@H]2C[C@](C)(NC(C)=O)C2)nn2cc(F)c(-c3ccc4nccn4n3)c12 ; CNc1nc(N[C@H]2C[C@@](C)(NC(C)=O)C2)nn2cc(F)c(-c3ccc4nccn4n3)c12 ; CNc1nc(N[C@H]2CC[C@@](C)(O)CC2)nn2cc(F)c(-c3ccc4nccn4n3)c12] 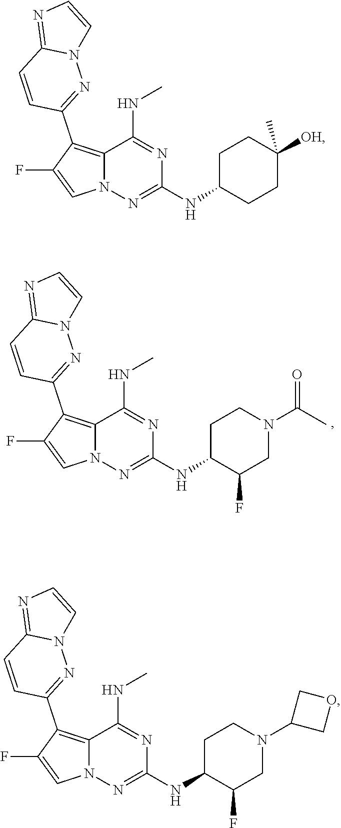 [SMILES: CNc1nc(N[C@H]2CC[C@](C)(O)CC2)nn2cc(F)c(-c3ccc4nccn4n3)c12 ; CNc1nc(N[C@@H]2CCN(C(C)=O)C[C@H]2F)nn2cc(F)c(-c3ccc4nccn4n3)c12 ; CNc1nc(N[C@H]2CCN(C3COC3)C[C@H]2F)nn2cc(F)c(-c3ccc4nccn4n3)c12] 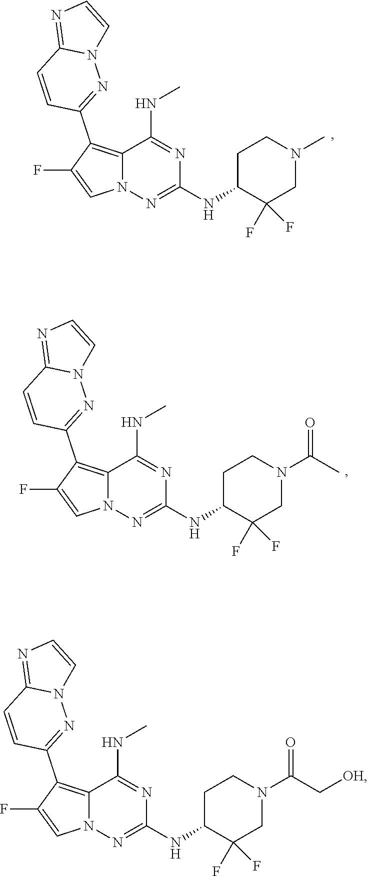 [SMILES: CNc1nc(N[C@@H]2CCN(C)CC2(F)F)nn2cc(F)c(-c3ccc4nccn4n3)c12 ; CNc1nc(N[C@@H]2CCN(C(C)=O)CC2(F)F)nn2cc(F)c(-c3ccc4nccn4n3)c12 ; CNc1nc(N[C@@H]2CCN(C(=O)CO)CC2(F)F)nn2cc(F)c(-c3ccc4nccn4n3)c12] 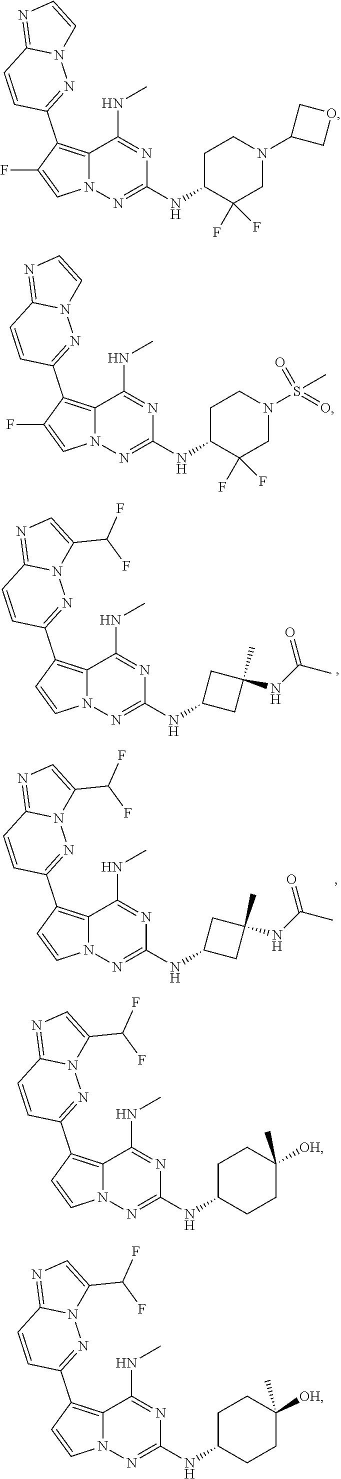 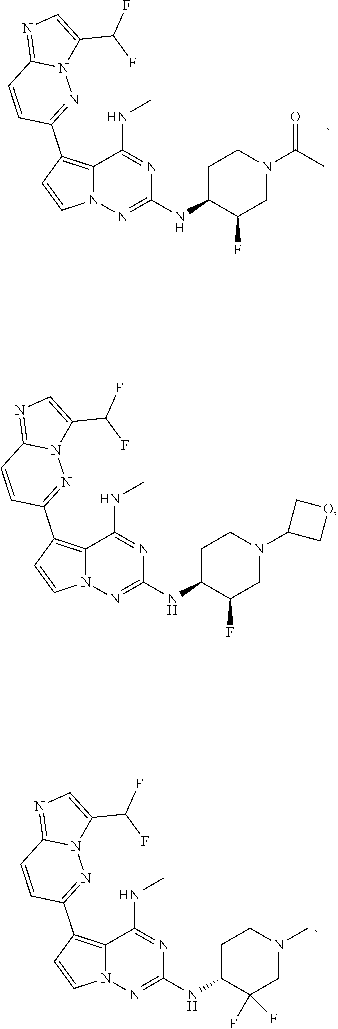 [SMILES: CNc1nc(N[C@H]2CCN(C(C)=O)C[C@H]2F)nn2ccc(-c3ccc4ncc(C(F)F)n4n3)c12 ; CNc1nc(N[C@H]2CCN(C3COC3)C[C@H]2F)nn2ccc(-c3ccc4ncc(C(F)F)n4n3)c12 ; CNc1nc(N[C@@H]2CCN(C)CC2(F)F)nn2ccc(-c3ccc4ncc(C(F)F)n4n3)c12] 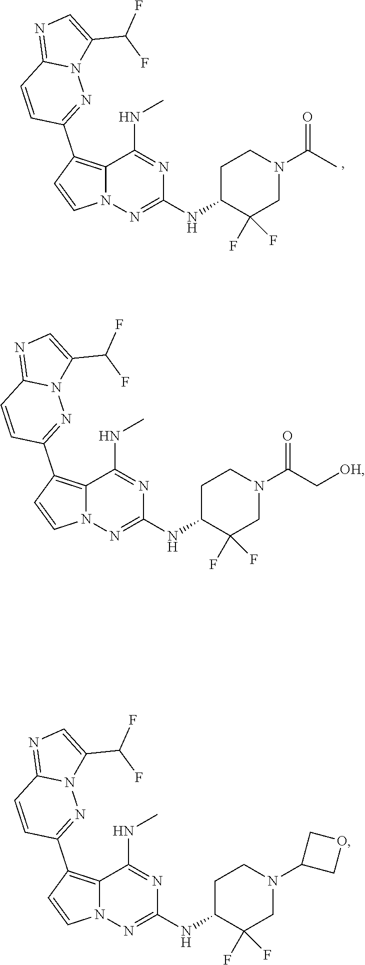 [SMILES: CNc1nc(N[C@@H]2CCN(C(C)=O)CC2(F)F)nn2ccc(-c3ccc4ncc(C(F)F)n4n3)c12 ; CNc1nc(N[C@@H]2CCN(C(=O)CO)CC2(F)F)nn2ccc(-c3ccc4ncc(C(F)F)n4n3)c12 ; CNc1nc(N[C@@H]2CCN(C3COC3)CC2(F)F)nn2ccc(-c3ccc4ncc(C(F)F)n4n3)c12] 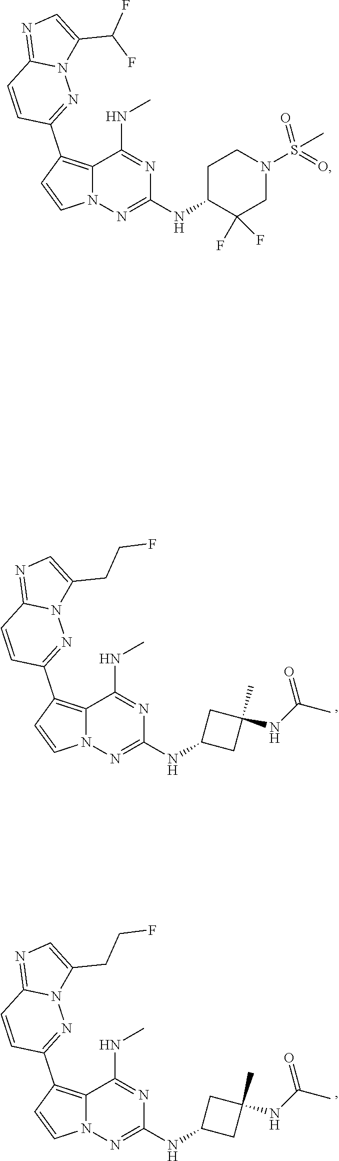 [SMILES: CNc1nc(N[C@@H]2CCN(S(C)(=O)=O)CC2(F)F)nn2ccc(-c3ccc4ncc(C(F)F)n4n3)c12 ; CNc1nc(N[C@H]2C[C@](C)(NC(C)=O)C2)nn2ccc(-c3ccc4ncc(CCF)n4n3)c12 ; CNc1nc(N[C@H]2C[C@@](C)(NC(C)=O)C2)nn2ccc(-c3ccc4ncc(CCF)n4n3)c12] 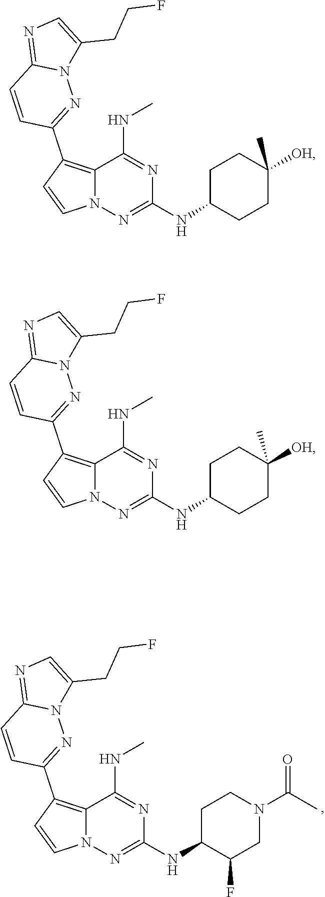 [SMILES: CNc1nc(N[C@H]2CC[C@@](C)(O)CC2)nn2ccc(-c3ccc4ncc(CCF)n4n3)c12 ; CNc1nc(N[C@H]2CC[C@](C)(O)CC2)nn2ccc(-c3ccc4ncc(CCF)n4n3)c12 ; CNc1nc(N[C@H]2CCN(C(C)=O)C[C@H]2F)nn2ccc(-c3ccc4ncc(CCF)n4n3)c12] 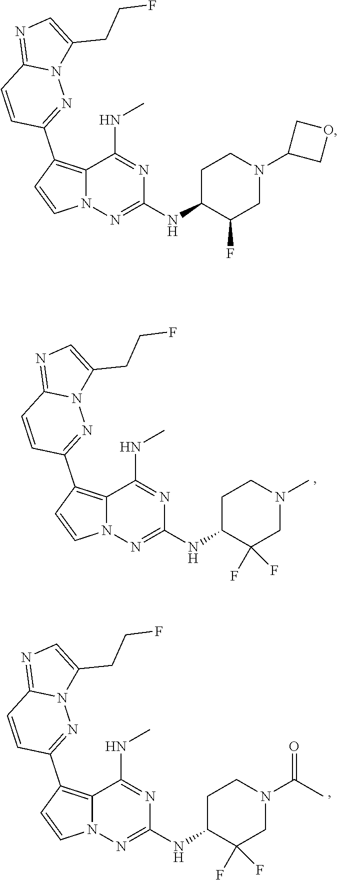 [SMILES: CNc1nc(N[C@H]2CCN(C3COC3)C[C@H]2F)nn2ccc(-c3ccc4ncc(CCF)n4n3)c12 ; CNc1nc(N[C@@H]2CCN(C)CC2(F)F)nn2ccc(-c3ccc4ncc(CCF)n4n3)c12 ; CNc1nc(N[C@@H]2CCN(C(C)=O)CC2(F)F)nn2ccc(-c3ccc4ncc(CCF)n4n3)c12] 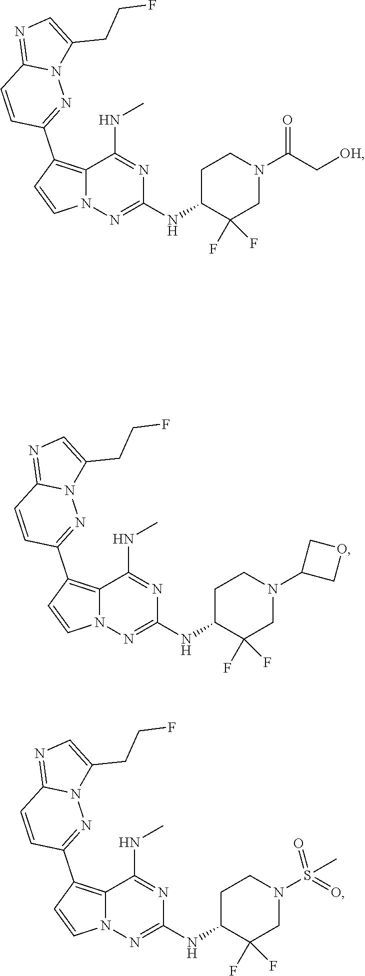 [SMILES: CNc1nc(N[C@@H]2CCN(C(=O)CO)CC2(F)F)nn2ccc(-c3ccc4ncc(CCF)n4n3)c12 ; CNc1nc(N[C@@H]2CCN(C3COC3)CC2(F)F)nn2ccc(-c3ccc4ncc(CCF)n4n3)c12 ; CNc1nc(N[C@@H]2CCN(S(C)(=O)=O)CC2(F)F)nn2ccc(-c3ccc4ncc(CCF)n4n3)c12] 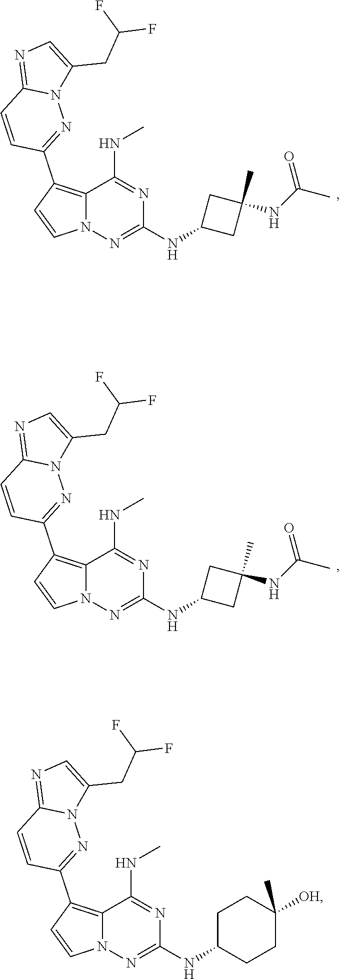 [SMILES: CNc1nc(N[C@H]2C[C@@](C)(NC(C)=O)C2)nn2ccc(-c3ccc4ncc(CC(F)F)n4n3)c12 ; CNc1nc(N[C@H]2C[C@](C)(NC(C)=O)C2)nn2ccc(-c3ccc4ncc(CC(F)F)n4n3)c12 ; CNc1nc(N[C@H]2CC[C@@](C)(O)CC2)nn2ccc(-c3ccc4ncc(CC(F)F)n4n3)c12] 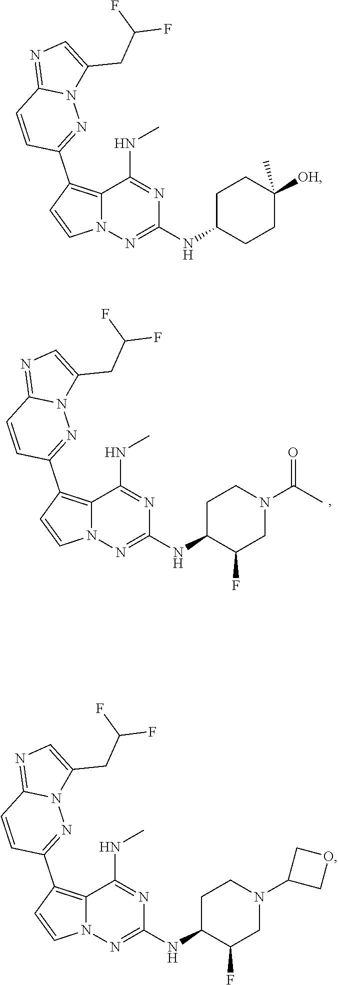 [SMILES: CNc1nc(N[C@H]2CC[C@](C)(O)CC2)nn2ccc(-c3ccc4ncc(CC(F)F)n4n3)c12 ; CNc1nc(N[C@H]2CCN(C(C)=O)C[C@H]2F)nn2ccc(-c3ccc4ncc(CC(F)F)n4n3)c12 ; CNc1nc(N[C@H]2CCN(C3COC3)C[C@H]2F)nn2ccc(-c3ccc4ncc(CC(F)F)n4n3)c12] 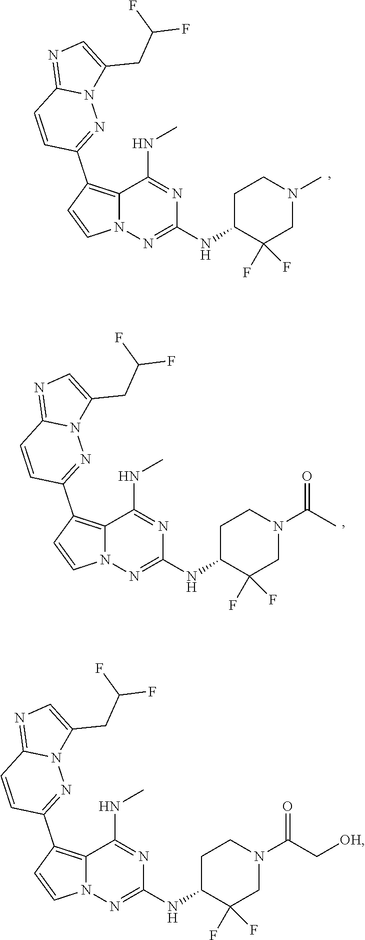 [SMILES: CNc1nc(N[C@@H]2CCN(C)CC2(F)F)nn2ccc(-c3ccc4ncc(CC(F)F)n4n3)c12 ; CNc1nc(N[C@@H]2CCN(C(C)=O)CC2(F)F)nn2ccc(-c3ccc4ncc(CC(F)F)n4n3)c12 ; CNc1nc(N[C@@H]2CCN(C(=O)CO)CC2(F)F)nn2ccc(-c3ccc4ncc(CC(F)F)n4n3)c12] 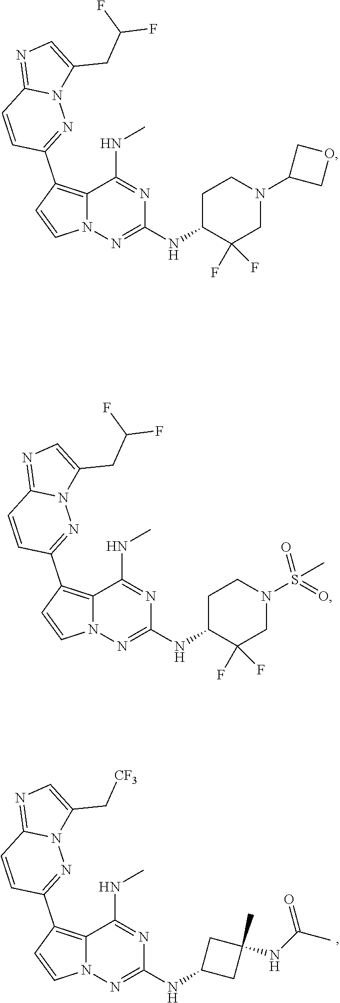 [SMILES: CNc1nc(N[C@@H]2CCN(C3COC3)CC2(F)F)nn2ccc(-c3ccc4ncc(CC(F)F)n4n3)c12 ; CNc1nc(N[C@@H]2CCN(S(C)(=O)=O)CC2(F)F)nn2ccc(-c3ccc4ncc(CC(F)F)n4n3)c12 ; CNc1nc(N[C@H]2C[C@@](C)(NC(C)=O)C2)nn2ccc(-c3ccc4ncc(CC(F)(F)F)n4n3)c12] 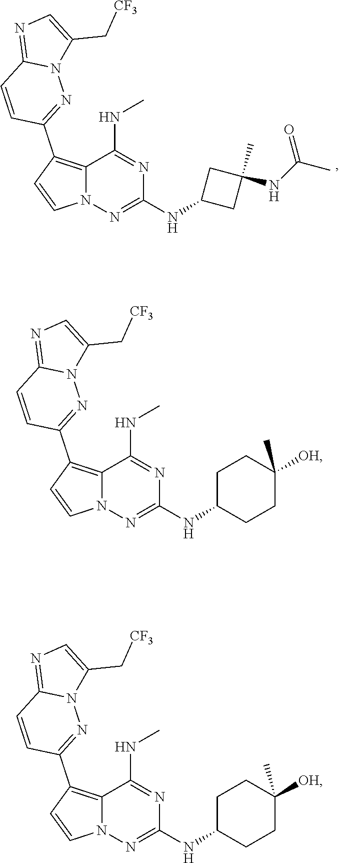 [SMILES: CNc1nc(N[C@H]2C[C@](C)(NC(C)=O)C2)nn2ccc(-c3ccc4ncc(CC(F)(F)F)n4n3)c12 ; CNc1nc(N[C@H]2CC[C@@](C)(O)CC2)nn2ccc(-c3ccc4ncc(CC(F)(F)F)n4n3)c12 ; CNc1nc(N[C@H]2CC[C@](C)(O)CC2)nn2ccc(-c3ccc4ncc(CC(F)(F)F)n4n3)c12] 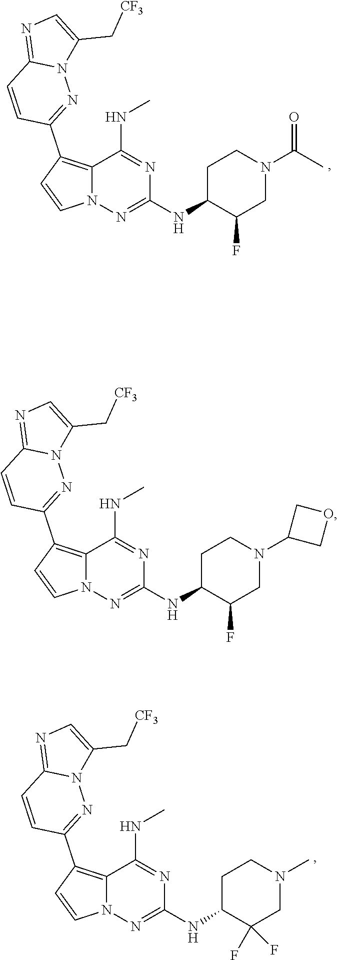 [SMILES: CNc1nc(N[C@H]2CCN(C(C)=O)C[C@H]2F)nn2ccc(-c3ccc4ncc(CC(F)(F)F)n4n3)c12 ; CNc1nc(N[C@H]2CCN(C3COC3)C[C@H]2F)nn2ccc(-c3ccc4ncc(CC(F)(F)F)n4n3)c12 ; CNc1nc(N[C@@H]2CCN(C)CC2(F)F)nn2ccc(-c3ccc4ncc(CC(F)(F)F)n4n3)c12] 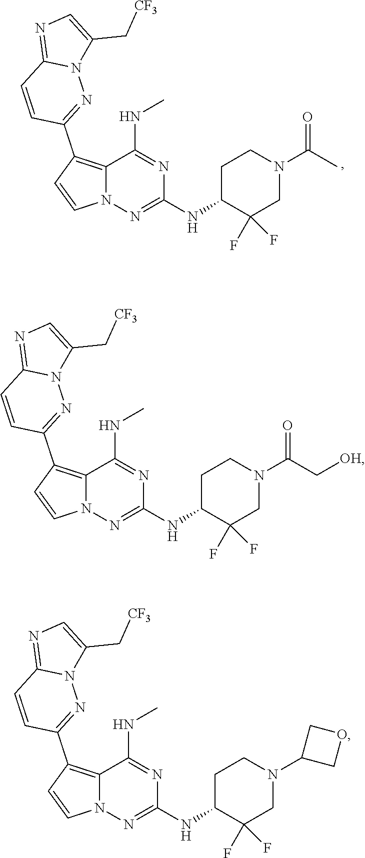 [SMILES: CNc1nc(N[C@@H]2CCN(C(C)=O)CC2(F)F)nn2ccc(-c3ccc4ncc(CC(F)(F)F)n4n3)c12 ; CNc1nc(N[C@@H]2CCN(C(=O)CO)CC2(F)F)nn2ccc(-c3ccc4ncc(CC(F)(F)F)n4n3)c12 ; CNc1nc(N[C@@H]2CCN(C3COC3)CC2(F)F)nn2ccc(-c3ccc4ncc(CC(F)(F)F)n4n3)c12] 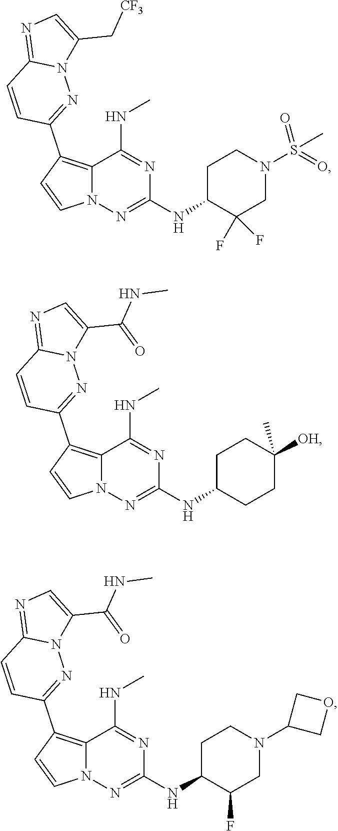 [SMILES: CNc1nc(N[C@@H]2CCN(S(C)(=O)=O)CC2(F)F)nn2ccc(-c3ccc4ncc(CC(F)(F)F)n4n3)c12 ; CNC(=O)c1cnc2ccc(-c3ccn4nc(N[C@H]5CC[C@](C)(O)CC5)nc(NC)c34)nn12 ; CNC(=O)c1cnc2ccc(-c3ccn4nc(N[C@H]5CCN(C6COC6)C[C@H]5F)nc(NC)c34)nn12] 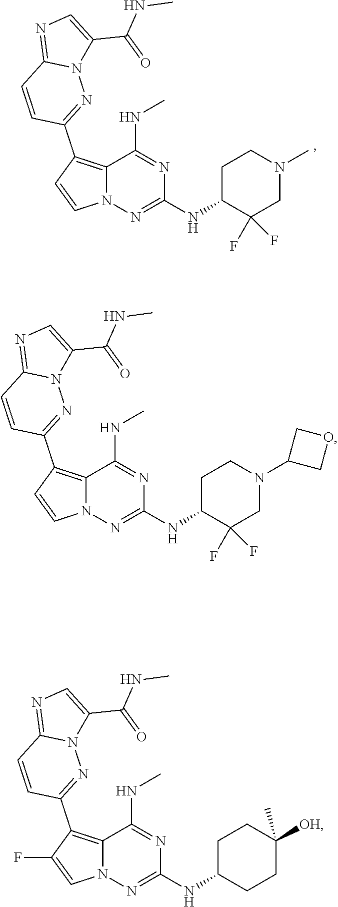 [SMILES: CNC(=O)c1cnc2ccc(-c3ccn4nc(N[C@@H]5CCN(C)CC5(F)F)nc(NC)c34)nn12 ; CNC(=O)c1cnc2ccc(-c3ccn4nc(N[C@@H]5CCN(C6COC6)CC5(F)F)nc(NC)c34)nn12 ; CNC(=O)c1cnc2ccc(-c3c(F)cn4nc(N[C@H]5CC[C@](C)(O)CC5)nc(NC)c34)nn12] 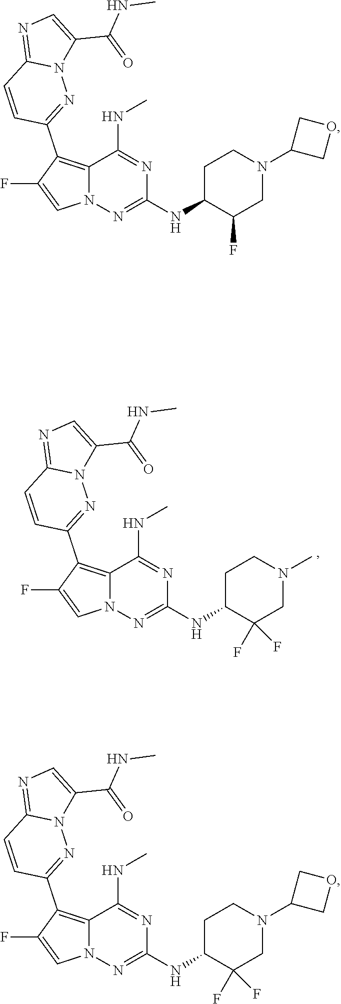 [SMILES: CNC(=O)c1cnc2ccc(-c3c(F)cn4nc(N[C@H]5CCN(C6COC6)C[C@H]5F)nc(NC)c34)nn12 ; CNC(=O)c1cnc2ccc(-c3c(F)cn4nc(N[C@@H]5CCN(C)CC5(F)F)nc(NC)c34)nn12 ; CNC(=O)c1cnc2ccc(-c3c(F)cn4nc(N[C@@H]5CCN(C6COC6)CC5(F)F)nc(NC)c34)nn12] 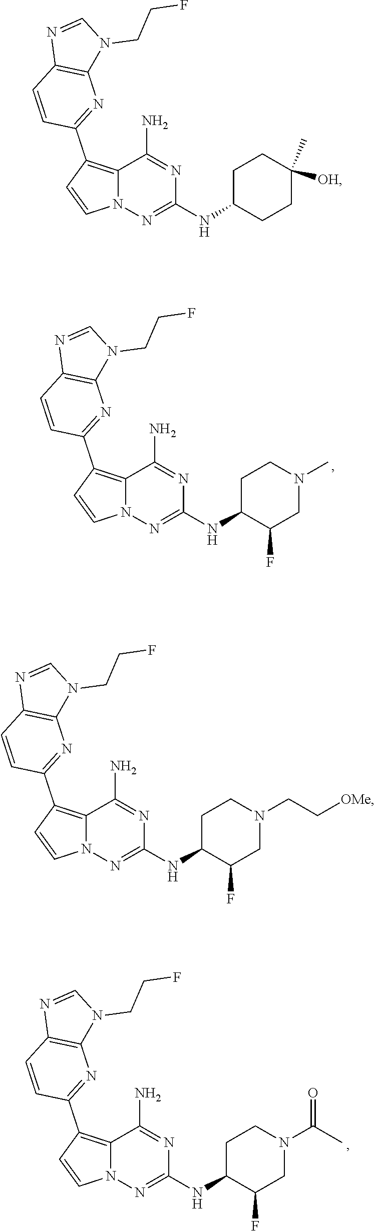 [SMILES: C[C@]1(O)CC[C@H](Nc2nc(N)c3c(-c4ccc5ncn(CCF)c5n4)ccn3n2)CC1 ; CN1CC[C@H](Nc2nc(N)c3c(-c4ccc5ncn(CCF)c5n4)ccn3n2)[C@H](F)C1 ; COCCN1CC[C@H](Nc2nc(N)c3c(-c4ccc5ncn(CCF)c5n4)ccn3n2)[C@H](F)C1 ; CC(=O)N1CC[C@H](Nc2nc(N)c3c(-c4ccc5ncn(CCF)c5n4)ccn3n2)[C@H](F)C1] 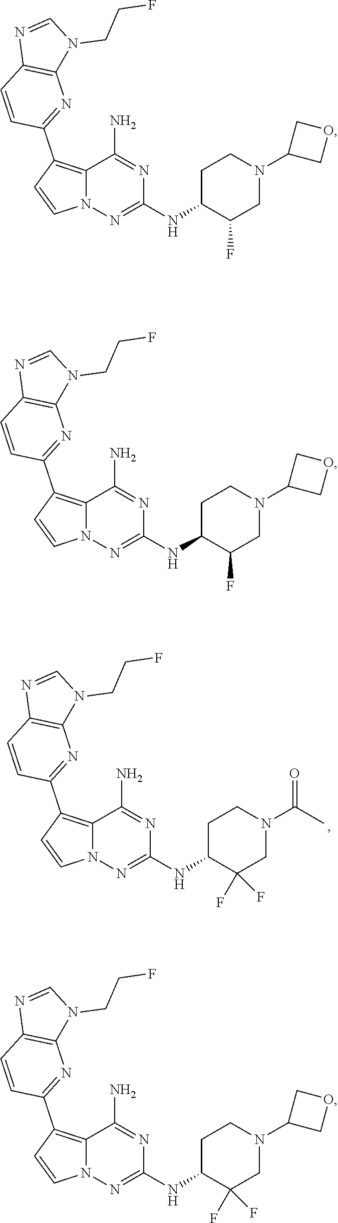 [SMILES: Nc1nc(N[C@@H]2CCN(C3COC3)C[C@@H]2F)nn2ccc(-c3ccc4ncn(CCF)c4n3)c12 ; Nc1nc(N[C@H]2CCN(C3COC3)C[C@H]2F)nn2ccc(-c3ccc4ncn(CCF)c4n3)c12 ; CC(=O)N1CC[C@@H](Nc2nc(N)c3c(-c4ccc5ncn(CCF)c5n4)ccn3n2)C(F)(F)C1 ; Nc1nc(N[C@@H]2CCN(C3COC3)CC2(F)F)nn2ccc(-c3ccc4ncn(CCF)c4n3)c12] 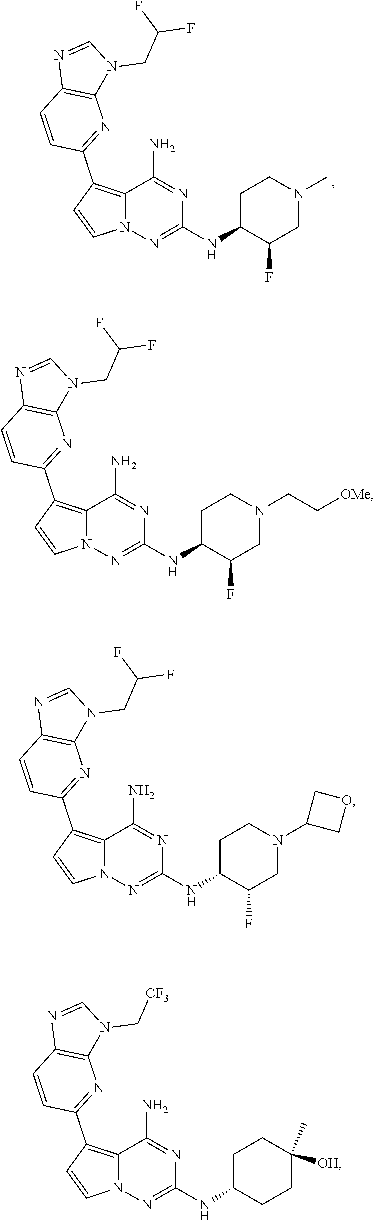 [SMILES: CN1CC[C@H](Nc2nc(N)c3c(-c4ccc5ncn(CC(F)F)c5n4)ccn3n2)[C@H](F)C1 ; COCCN1CC[C@H](Nc2nc(N)c3c(-c4ccc5ncn(CC(F)F)c5n4)ccn3n2)[C@H](F)C1 ; Nc1nc(N[C@@H]2CCN(C3COC3)C[C@@H]2F)nn2ccc(-c3ccc4ncn(CC(F)F)c4n3)c12 ; C[C@]1(O)CC[C@H](Nc2nc(N)c3c(-c4ccc5ncn(CC(F)(F)F)c5n4)ccn3n2)CC1] 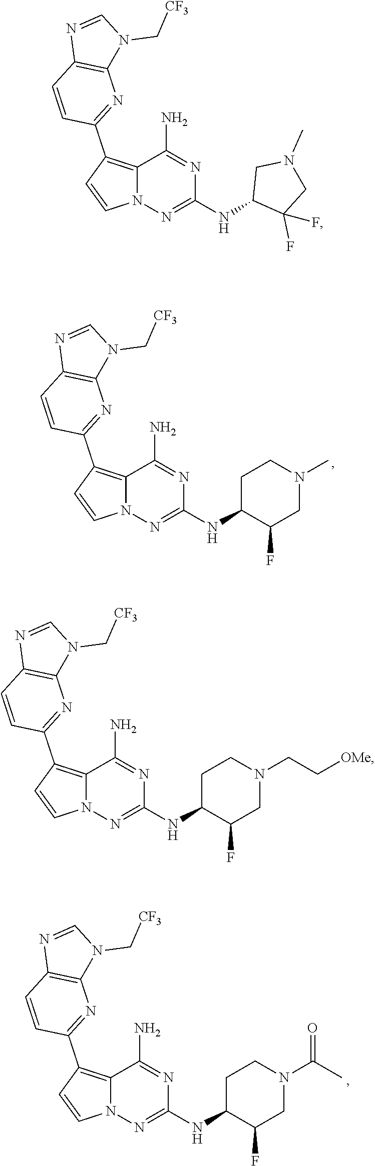 [SMILES: CN1C[C@@H](Nc2nc(N)c3c(-c4ccc5ncn(CC(F)(F)F)c5n4)ccn3n2)C(F)(F)C1 ; CN1CC[C@H](Nc2nc(N)c3c(-c4ccc5ncn(CC(F)(F)F)c5n4)ccn3n2)[C@H](F)C1 ; COCCN1CC[C@H](Nc2nc(N)c3c(-c4ccc5ncn(CC(F)(F)F)c5n4)ccn3n2)[C@H](F)C1 ; CC(=O)N1CC[C@H](Nc2nc(N)c3c(-c4ccc5ncn(CC(F)(F)F)c5n4)ccn3n2)[C@H](F)C1] 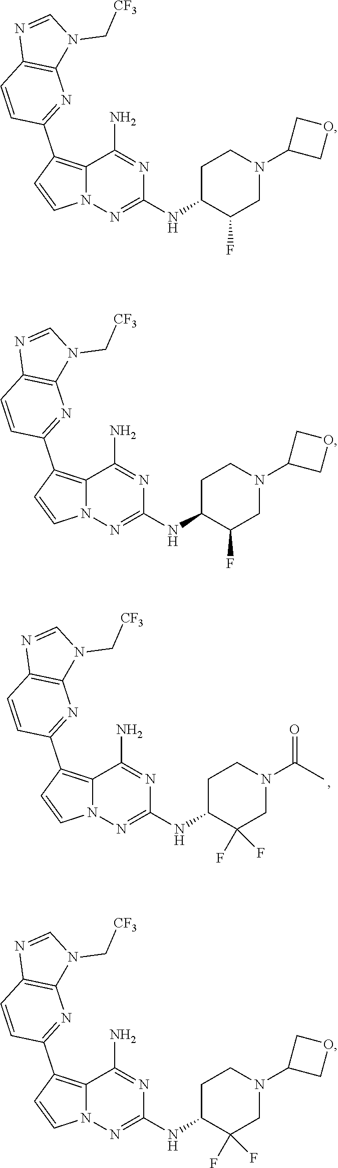 [SMILES: Nc1nc(N[C@@H]2CCN(C3COC3)C[C@@H]2F)nn2ccc(-c3ccc4ncn(CC(F)(F)F)c4n3)c12 ; Nc1nc(N[C@H]2CCN(C3COC3)C[C@H]2F)nn2ccc(-c3ccc4ncn(CC(F)(F)F)c4n3)c12 ; CC(=O)N1CC[C@@H](Nc2nc(N)c3c(-c4ccc5ncn(CC(F)(F)F)c5n4)ccn3n2)C(F)(F)C1 ; Nc1nc(N[C@@H]2CCN(C3COC3)CC2(F)F)nn2ccc(-c3ccc4ncn(CC(F)(F)F)c4n3)c12] 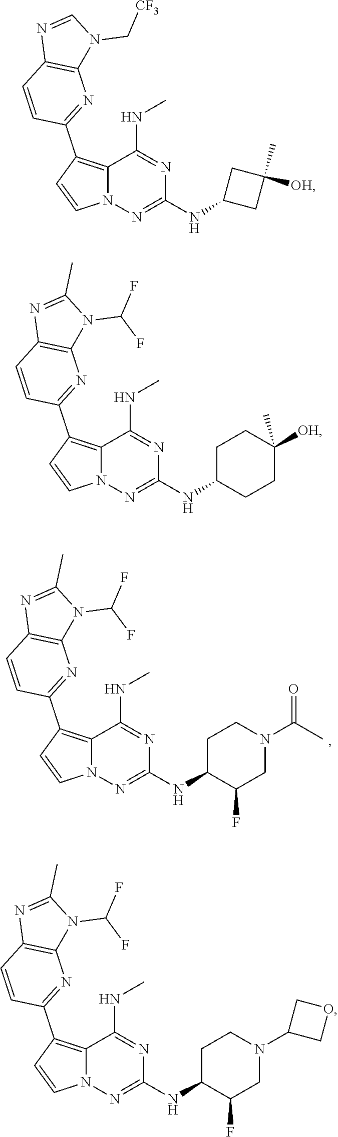 [SMILES: CNc1nc(N[C@H]2C[C@](C)(O)C2)nn2ccc(-c3ccc4ncn(CC(F)(F)F)c4n3)c12 ; CNc1nc(N[C@H]2CC[C@](C)(O)CC2)nn2ccc(-c3ccc4nc(C)n(C(F)F)c4n3)c12 ; CNc1nc(N[C@H]2CCN(C(C)=O)C[C@H]2F)nn2ccc(-c3ccc4nc(C)n(C(F)F)c4n3)c12 ; CNc1nc(N[C@H]2CCN(C3COC3)C[C@H]2F)nn2ccc(-c3ccc4nc(C)n(C(F)F)c4n3)c12] 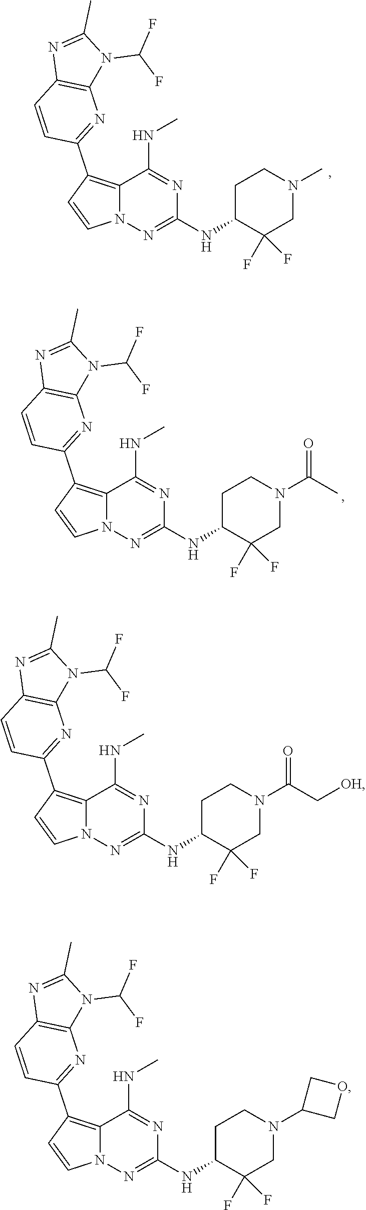 [SMILES: CNc1nc(N[C@@H]2CCN(C)CC2(F)F)nn2ccc(-c3ccc4nc(C)n(C(F)F)c4n3)c12 ; CNc1nc(N[C@@H]2CCN(C(C)=O)CC2(F)F)nn2ccc(-c3ccc4nc(C)n(C(F)F)c4n3)c12 ; CNc1nc(N[C@@H]2CCN(C(=O)CO)CC2(F)F)nn2ccc(-c3ccc4nc(C)n(C(F)F)c4n3)c12 ; CNc1nc(N[C@@H]2CCN(C3COC3)CC2(F)F)nn2ccc(-c3ccc4nc(C)n(C(F)F)c4n3)c12] 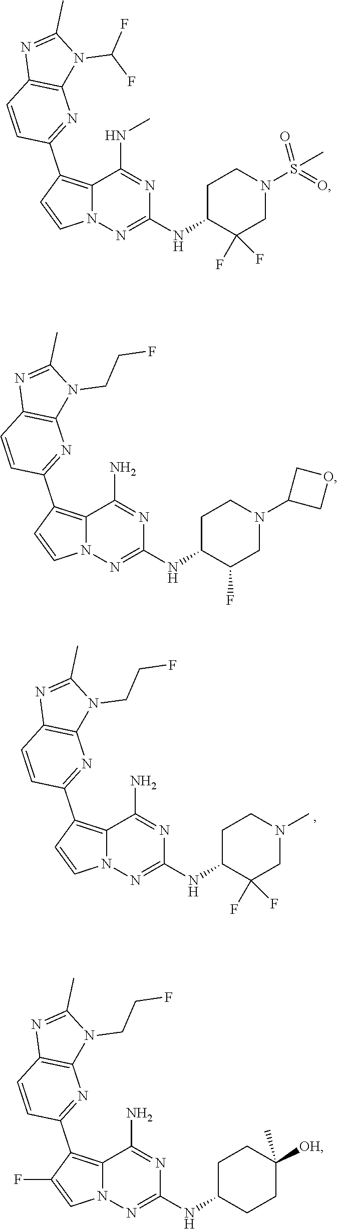 [SMILES: CNc1nc(N[C@@H]2CCN(S(C)(=O)=O)CC2(F)F)nn2ccc(-c3ccc4nc(C)n(C(F)F)c4n3)c12 ; Cc1nc2ccc(-c3ccn4nc(N[C@@H]5CCN(C6COC6)C[C@@H]5F)nc(N)c34)nc2n1CCF ; Cc1nc2ccc(-c3ccn4nc(N[C@@H]5CCN(C)CC5(F)F)nc(N)c34)nc2n1CCF ; Cc1nc2ccc(-c3c(F)cn4nc(N[C@H]5CC[C@](C)(O)CC5)nc(N)c34)nc2n1CCF] 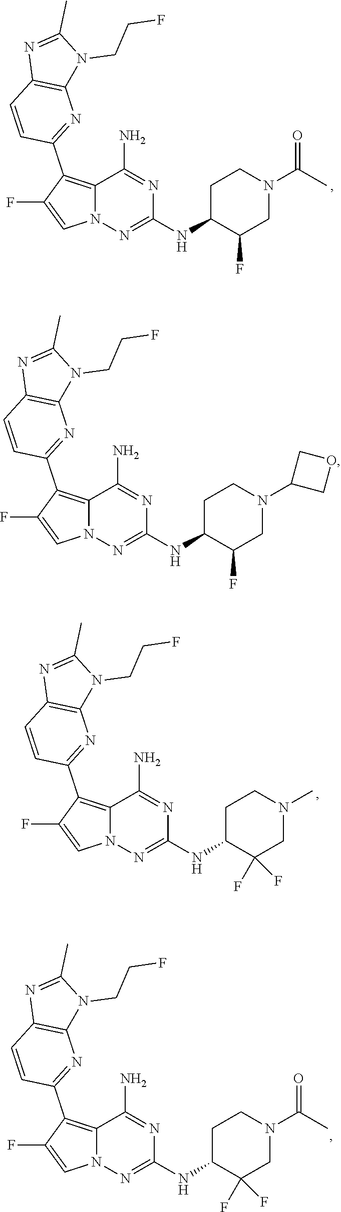 [SMILES: CC(=O)N1CC[C@H](Nc2nc(N)c3c(-c4ccc5nc(C)n(CCF)c5n4)c(F)cn3n2)[C@H](F)C1 ; Cc1nc2ccc(-c3c(F)cn4nc(N[C@H]5CCN(C6COC6)C[C@H]5F)nc(N)c34)nc2n1CCF ; Cc1nc2ccc(-c3c(F)cn4nc(N[C@@H]5CCN(C)CC5(F)F)nc(N)c34)nc2n1CCF ; CC(=O)N1CC[C@@H](Nc2nc(N)c3c(-c4ccc5nc(C)n(CCF)c5n4)c(F)cn3n2)C(F)(F)C1] 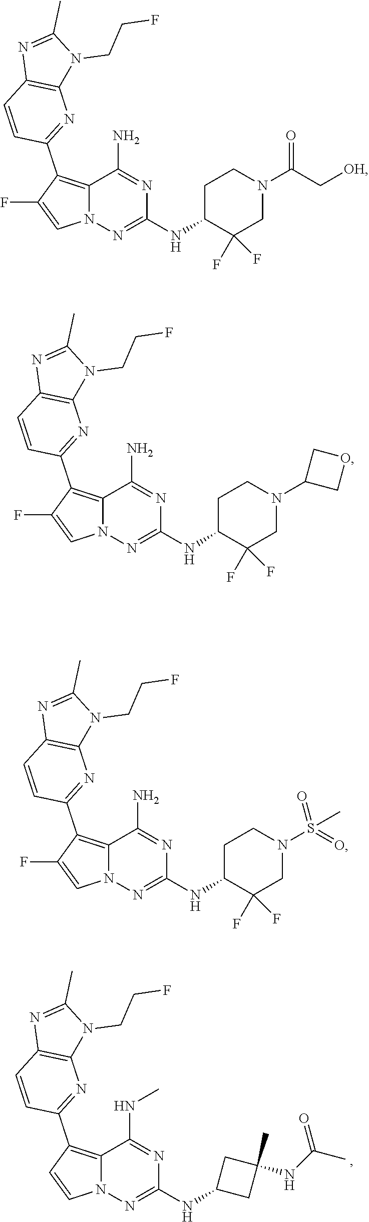 [SMILES: Cc1nc2ccc(-c3c(F)cn4nc(N[C@@H]5CCN(C(=O)CO)CC5(F)F)nc(N)c34)nc2n1CCF ; Cc1nc2ccc(-c3c(F)cn4nc(N[C@@H]5CCN(C6COC6)CC5(F)F)nc(N)c34)nc2n1CCF ; Cc1nc2ccc(-c3c(F)cn4nc(N[C@@H]5CCN(S(C)(=O)=O)CC5(F)F)nc(N)c34)nc2n1CCF ; CNc1nc(N[C@H]2C[C@@](C)(NC(C)=O)C2)nn2ccc(-c3ccc4nc(C)n(CCF)c4n3)c12] 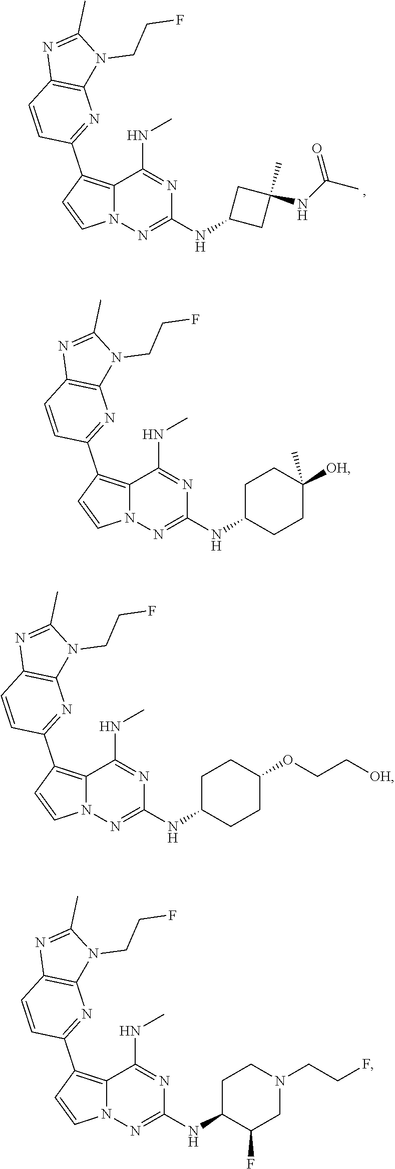 [SMILES: CNc1nc(N[C@H]2C[C@](C)(NC(C)=O)C2)nn2ccc(-c3ccc4nc(C)n(CCF)c4n3)c12 ; CNc1nc(N[C@H]2CC[C@](C)(O)CC2)nn2ccc(-c3ccc4nc(C)n(CCF)c4n3)c12 ; CNc1nc(N[C@H]2CC[C@@H](OCCO)CC2)nn2ccc(-c3ccc4nc(C)n(CCF)c4n3)c12 ; CNc1nc(N[C@H]2CCN(CCF)C[C@H]2F)nn2ccc(-c3ccc4nc(C)n(CCF)c4n3)c12] 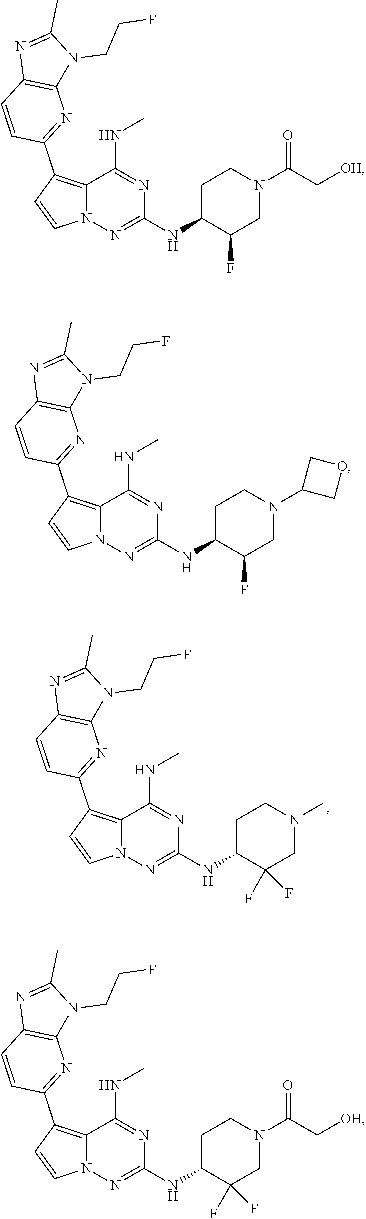 [SMILES: CNc1nc(N[C@H]2CCN(C(=O)CO)C[C@H]2F)nn2ccc(-c3ccc4nc(C)n(CCF)c4n3)c12 ; CNc1nc(N[C@H]2CCN(C3COC3)C[C@H]2F)nn2ccc(-c3ccc4nc(C)n(CCF)c4n3)c12 ; CNc1nc(N[C@@H]2CCN(C)CC2(F)F)nn2ccc(-c3ccc4nc(C)n(CCF)c4n3)c12 ; CNc1nc(N[C@@H]2CCN(C(=O)CO)CC2(F)F)nn2ccc(-c3ccc4nc(C)n(CCF)c4n3)c12] 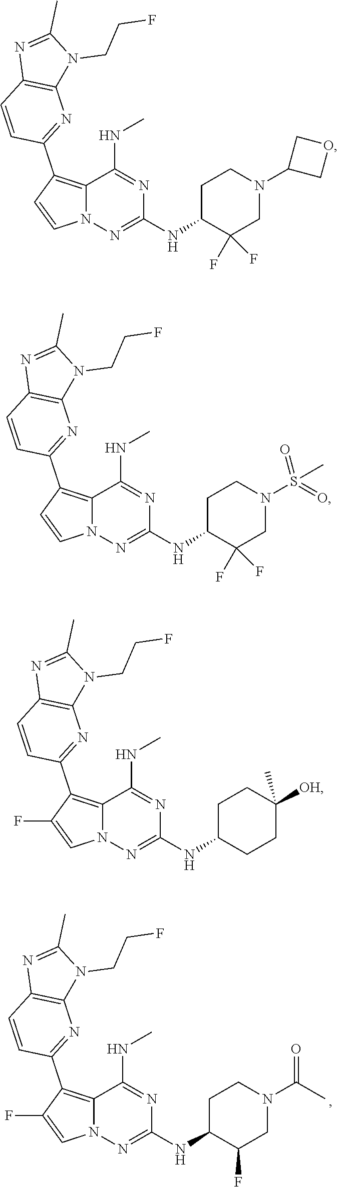 [SMILES: CNc1nc(N[C@@H]2CCN(C3COC3)CC2(F)F)nn2ccc(-c3ccc4nc(C)n(CCF)c4n3)c12 ; CNc1nc(N[C@@H]2CCN(S(C)(=O)=O)CC2(F)F)nn2ccc(-c3ccc4nc(C)n(CCF)c4n3)c12 ; CNc1nc(N[C@H]2CC[C@](C)(O)CC2)nn2cc(F)c(-c3ccc4nc(C)n(CCF)c4n3)c12 ; CNc1nc(N[C@H]2CCN(C(C)=O)C[C@H]2F)nn2cc(F)c(-c3ccc4nc(C)n(CCF)c4n3)c12] 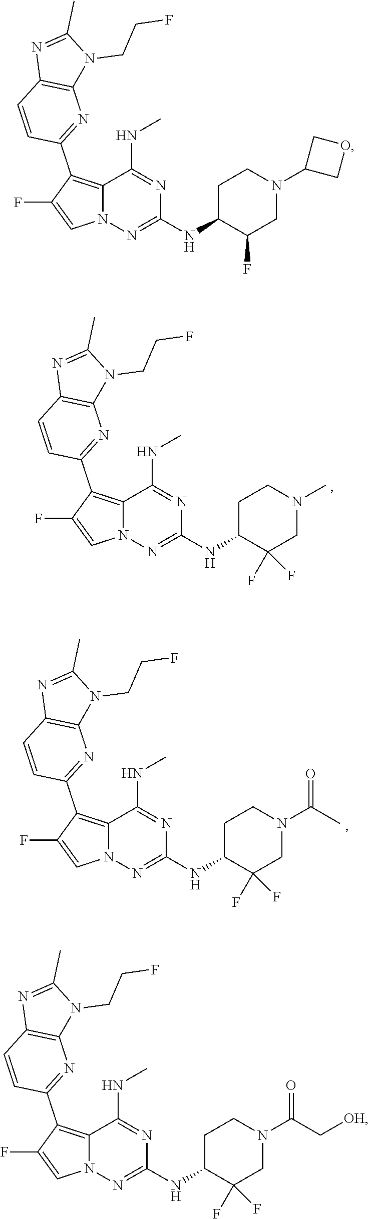 [SMILES: CNc1nc(N[C@H]2CCN(C3COC3)C[C@H]2F)nn2cc(F)c(-c3ccc4nc(C)n(CCF)c4n3)c12 ; CNc1nc(N[C@@H]2CCN(C)CC2(F)F)nn2cc(F)c(-c3ccc4nc(C)n(CCF)c4n3)c12 ; CNc1nc(N[C@@H]2CCN(C(C)=O)CC2(F)F)nn2cc(F)c(-c3ccc4nc(C)n(CCF)c4n3)c12 ; CNc1nc(N[C@@H]2CCN(C(=O)CO)CC2(F)F)nn2cc(F)c(-c3ccc4nc(C)n(CCF)c4n3)c12] 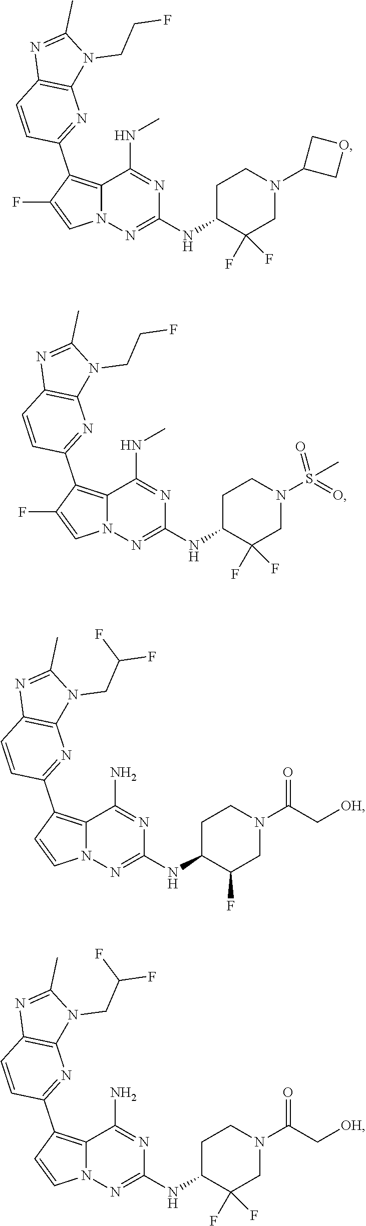 [SMILES: CNc1nc(N[C@@H]2CCN(C3COC3)CC2(F)F)nn2cc(F)c(-c3ccc4nc(C)n(CCF)c4n3)c12 ; CNc1nc(N[C@@H]2CCN(S(C)(=O)=O)CC2(F)F)nn2cc(F)c(-c3ccc4nc(C)n(CCF)c4n3)c12 ; Cc1nc2ccc(-c3ccn4nc(N[C@H]5CCN(C(=O)CO)C[C@H]5F)nc(N)c34)nc2n1CC(F)F ; Cc1nc2ccc(-c3ccn4nc(N[C@@H]5CCN(C(=O)CO)CC5(F)F)nc(N)c34)nc2n1CC(F)F] 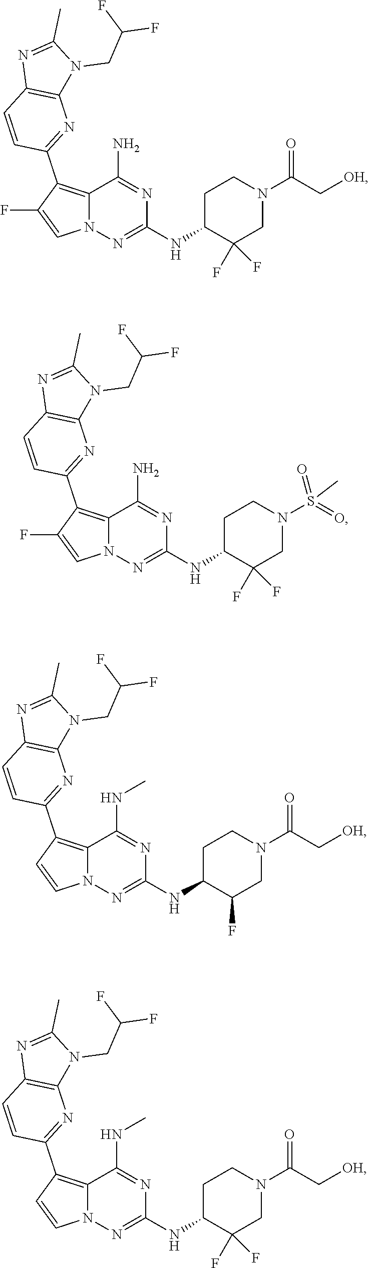 [SMILES: Cc1nc2ccc(-c3c(F)cn4nc(N[C@@H]5CCN(C(=O)CO)CC5(F)F)nc(N)c34)nc2n1CC(F)F ; Cc1nc2ccc(-c3c(F)cn4nc(N[C@@H]5CCN(S(C)(=O)=O)CC5(F)F)nc(N)c34)nc2n1CC(F)F ; CNc1nc(N[C@H]2CCN(C(=O)CO)C[C@H]2F)nn2ccc(-c3ccc4nc(C)n(CC(F)F)c4n3)c12 ; CNc1nc(N[C@@H]2CCN(C(=O)CO)CC2(F)F)nn2ccc(-c3ccc4nc(C)n(CC(F)F)c4n3)c12] 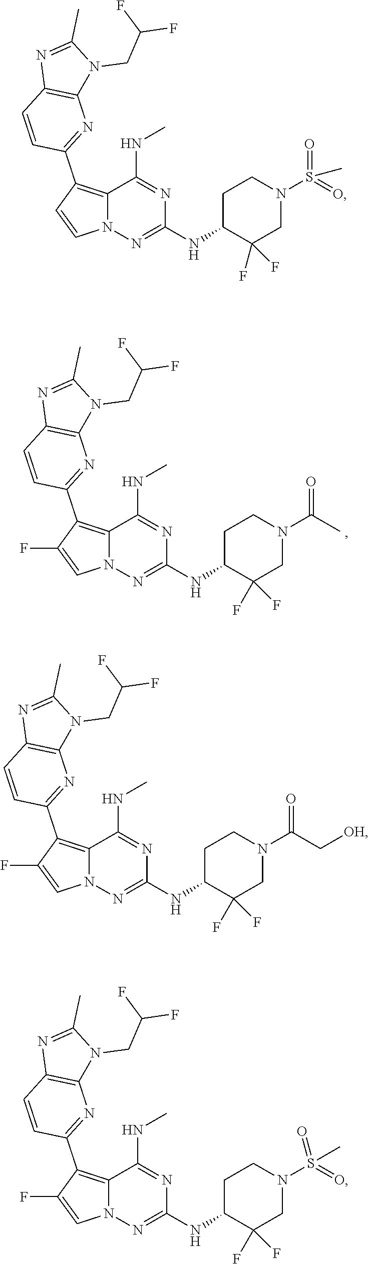 [SMILES: CNc1nc(N[C@@H]2CCN(S(C)(=O)=O)CC2(F)F)nn2ccc(-c3ccc4nc(C)n(CC(F)F)c4n3)c12 ; CNc1nc(N[C@@H]2CCN(C(C)=O)CC2(F)F)nn2cc(F)c(-c3ccc4nc(C)n(CC(F)F)c4n3)c12 ; CNc1nc(N[C@@H]2CCN(C(=O)CO)CC2(F)F)nn2cc(F)c(-c3ccc4nc(C)n(CC(F)F)c4n3)c12 ; CNc1nc(N[C@@H]2CCN(S(C)(=O)=O)CC2(F)F)nn2cc(F)c(-c3ccc4nc(C)n(CC(F)F)c4n3)c12] 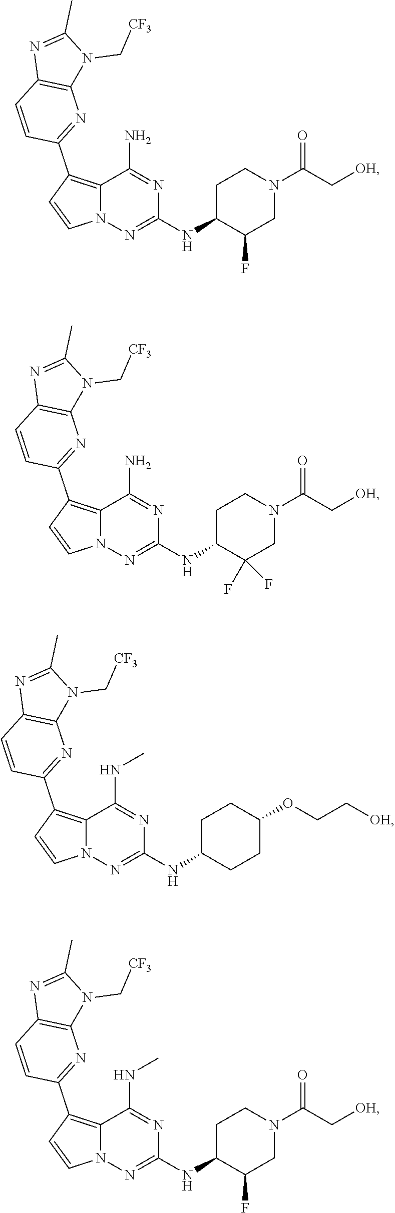 [SMILES: Cc1nc2ccc(-c3ccn4nc(N[C@H]5CCN(C(=O)CO)C[C@H]5F)nc(N)c34)nc2n1CC(F)(F)F ; Cc1nc2ccc(-c3ccn4nc(N[C@@H]5CCN(C(=O)CO)CC5(F)F)nc(N)c34)nc2n1CC(F)(F)F ; CNc1nc(N[C@H]2CC[C@@H](OCCO)CC2)nn2ccc(-c3ccc4nc(C)n(CC(F)(F)F)c4n3)c12 ; CNc1nc(N[C@H]2CCN(C(=O)CO)C[C@H]2F)nn2ccc(-c3ccc4nc(C)n(CC(F)(F)F)c4n3)c12] 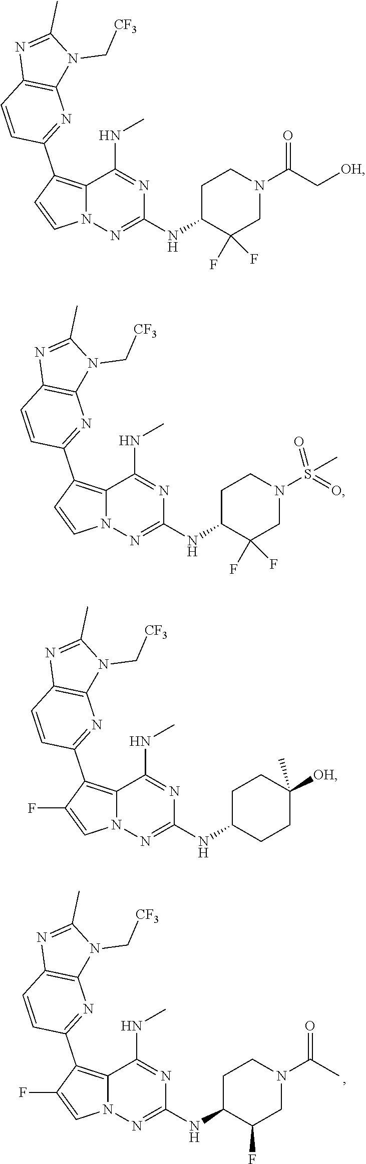 [SMILES: CNc1nc(N[C@@H]2CCN(C(=O)CO)CC2(F)F)nn2ccc(-c3ccc4nc(C)n(CC(F)(F)F)c4n3)c12 ; CNc1nc(N[C@@H]2CCN(S(C)(=O)=O)CC2(F)F)nn2ccc(-c3ccc4nc(C)n(CC(F)(F)F)c4n3)c12 ; CNc1nc(N[C@H]2CC[C@](C)(O)CC2)nn2cc(F)c(-c3ccc4nc(C)n(CC(F)(F)F)c4n3)c12 ; CNc1nc(N[C@H]2CCN(C(C)=O)C[C@H]2F)nn2cc(F)c(-c3ccc4nc(C)n(CC(F)(F)F)c4n3)c12] 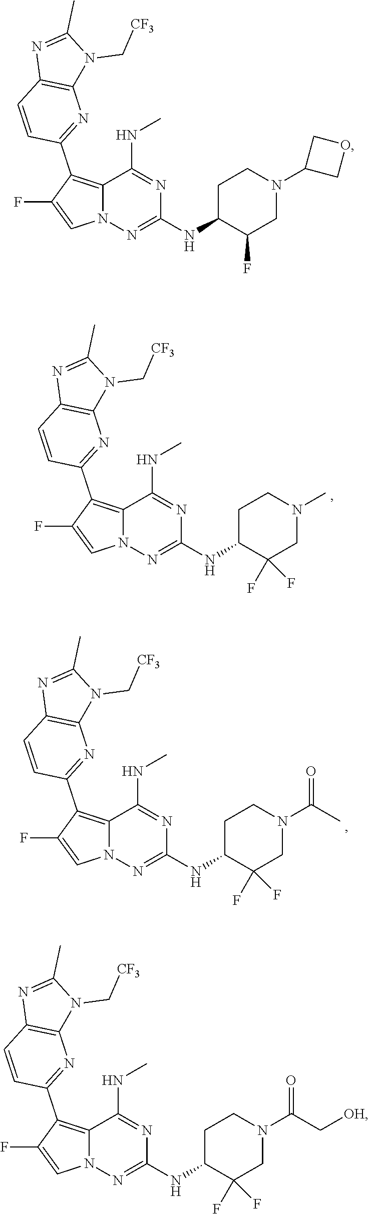 [SMILES: CNc1nc(N[C@H]2CCN(C3COC3)C[C@H]2F)nn2cc(F)c(-c3ccc4nc(C)n(CC(F)(F)F)c4n3)c12 ; CNc1nc(N[C@@H]2CCN(C)CC2(F)F)nn2cc(F)c(-c3ccc4nc(C)n(CC(F)(F)F)c4n3)c12 ; CNc1nc(N[C@@H]2CCN(C(C)=O)CC2(F)F)nn2cc(F)c(-c3ccc4nc(C)n(CC(F)(F)F)c4n3)c12 ; CNc1nc(N[C@@H]2CCN(C(=O)CO)CC2(F)F)nn2cc(F)c(-c3ccc4nc(C)n(CC(F)(F)F)c4n3)c12] 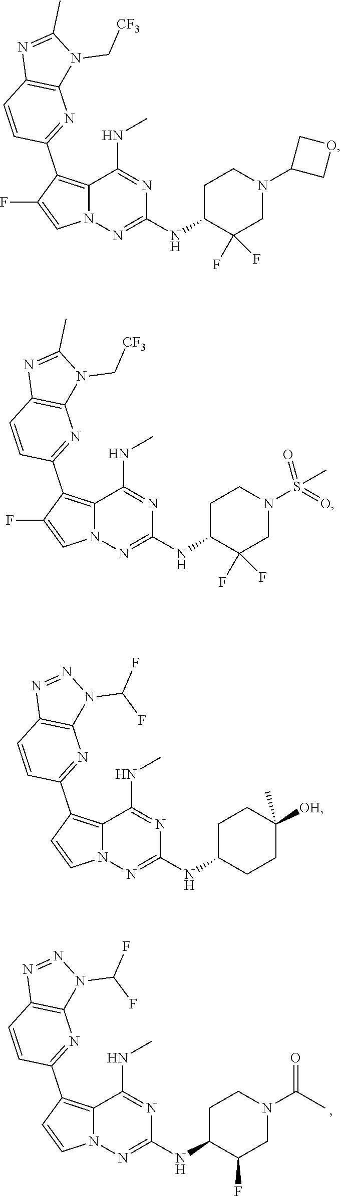 [SMILES: CNc1nc(N[C@@H]2CCN(C3COC3)CC2(F)F)nn2cc(F)c(-c3ccc4nc(C)n(CC(F)(F)F)c4n3)c12 ; CNc1nc(N[C@@H]2CCN(S(C)(=O)=O)CC2(F)F)nn2cc(F)c(-c3ccc4nc(C)n(CC(F)(F)F)c4n3)c12 ; CNc1nc(N[C@H]2CC[C@](C)(O)CC2)nn2ccc(-c3ccc4nnn(C(F)F)c4n3)c12 ; CNc1nc(N[C@H]2CCN(C(C)=O)C[C@H]2F)nn2ccc(-c3ccc4nnn(C(F)F)c4n3)c12] 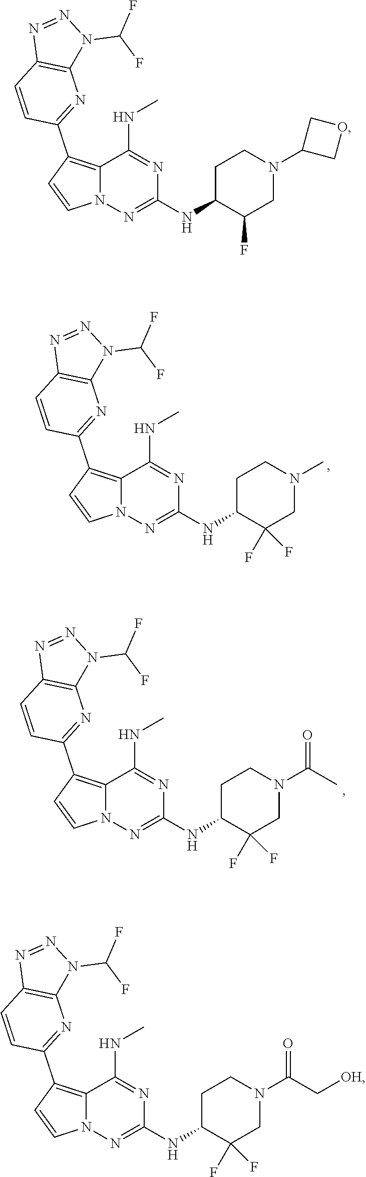 [SMILES: CNc1nc(N[C@H]2CCN(C3COC3)C[C@H]2F)nn2ccc(-c3ccc4nnn(C(F)F)c4n3)c12 ; CNc1nc(N[C@@H]2CCN(C)CC2(F)F)nn2ccc(-c3ccc4nnn(C(F)F)c4n3)c12 ; CNc1nc(N[C@@H]2CCN(C(C)=O)CC2(F)F)nn2ccc(-c3ccc4nnn(C(F)F)c4n3)c12 ; CNc1nc(N[C@@H]2CCN(C(=O)CO)CC2(F)F)nn2ccc(-c3ccc4nnn(C(F)F)c4n3)c12] 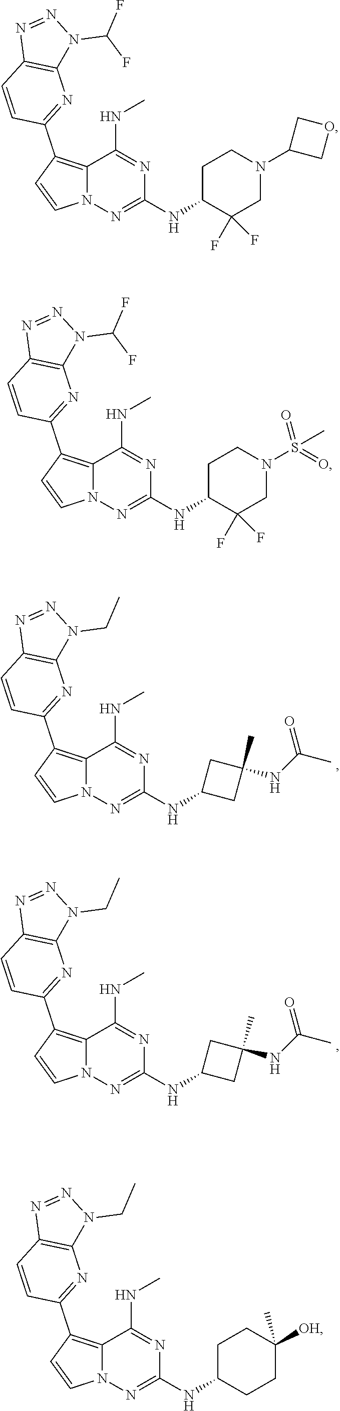 [SMILES: CNc1nc(N[C@@H]2CCN(C3COC3)CC2(F)F)nn2ccc(-c3ccc4nnn(C(F)F)c4n3)c12 ; CNc1nc(N[C@@H]2CCN(S(C)(=O)=O)CC2(F)F)nn2ccc(-c3ccc4nnn(C(F)F)c4n3)c12 ; CCn1nnc2ccc(-c3ccn4nc(N[C@H]5C[C@@](C)(NC(C)=O)C5)nc(NC)c34)nc21 ; CCn1nnc2ccc(-c3ccn4nc(N[C@H]5C[C@](C)(NC(C)=O)C5)nc(NC)c34)nc21 ; CCn1nnc2ccc(-c3ccn4nc(N[C@H]5CC[C@](C)(O)CC5)nc(NC)c34)nc21] 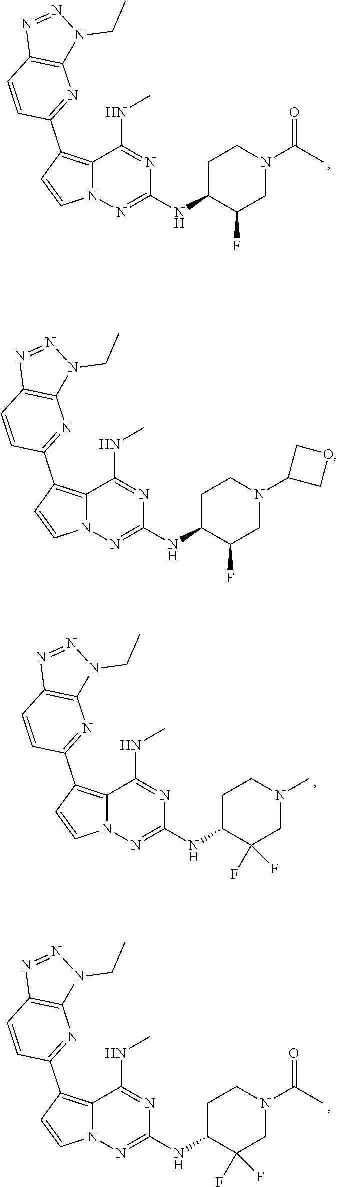 [SMILES: CCn1nnc2ccc(-c3ccn4nc(N[C@H]5CCN(C(C)=O)C[C@H]5F)nc(NC)c34)nc21 ; CCn1nnc2ccc(-c3ccn4nc(N[C@H]5CCN(C6COC6)C[C@H]5F)nc(NC)c34)nc21 ; CCn1nnc2ccc(-c3ccn4nc(N[C@@H]5CCN(C)CC5(F)F)nc(NC)c34)nc21 ; CCn1nnc2ccc(-c3ccn4nc(N[C@@H]5CCN(C(C)=O)CC5(F)F)nc(NC)c34)nc21] 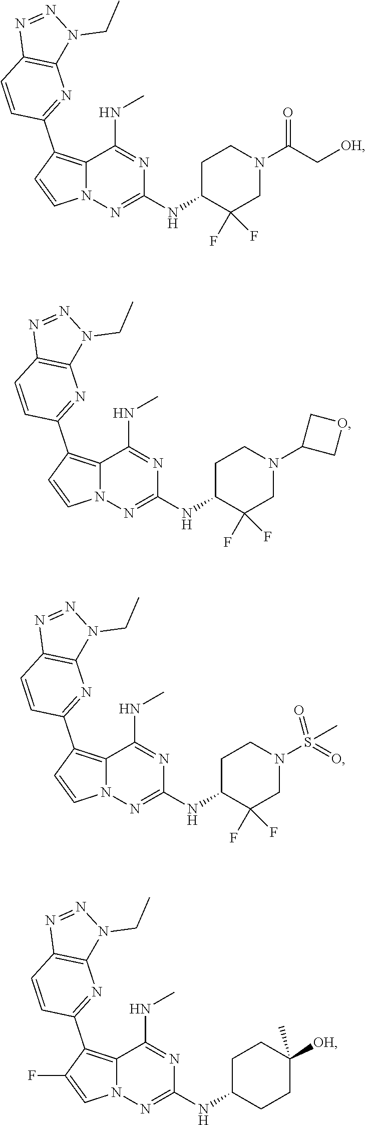 [SMILES: CCn1nnc2ccc(-c3ccn4nc(N[C@@H]5CCN(C(=O)CO)CC5(F)F)nc(NC)c34)nc21 ; CCn1nnc2ccc(-c3ccn4nc(N[C@@H]5CCN(C6COC6)CC5(F)F)nc(NC)c34)nc21 ; CCn1nnc2ccc(-c3ccn4nc(N[C@@H]5CCN(S(C)(=O)=O)CC5(F)F)nc(NC)c34)nc21 ; CCn1nnc2ccc(-c3c(F)cn4nc(N[C@H]5CC[C@](C)(O)CC5)nc(NC)c34)nc21] 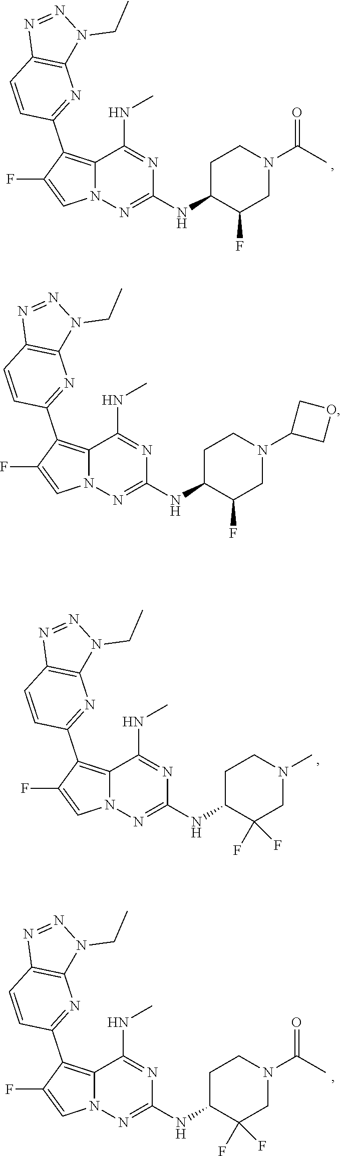 [SMILES: CCn1nnc2ccc(-c3c(F)cn4nc(N[C@H]5CCN(C(C)=O)C[C@H]5F)nc(NC)c34)nc21 ; CCn1nnc2ccc(-c3c(F)cn4nc(N[C@H]5CCN(C6COC6)C[C@H]5F)nc(NC)c34)nc21 ; CCn1nnc2ccc(-c3c(F)cn4nc(N[C@@H]5CCN(C)CC5(F)F)nc(NC)c34)nc21 ; CCn1nnc2ccc(-c3c(F)cn4nc(N[C@@H]5CCN(C(C)=O)CC5(F)F)nc(NC)c34)nc21] 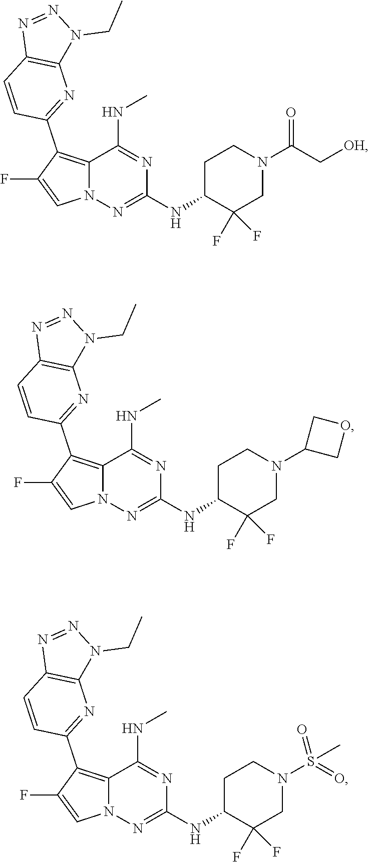 [SMILES: CCn1nnc2ccc(-c3c(F)cn4nc(N[C@@H]5CCN(C(=O)CO)CC5(F)F)nc(NC)c34)nc21 ; CCn1nnc2ccc(-c3c(F)cn4nc(N[C@@H]5CCN(C6COC6)CC5(F)F)nc(NC)c34)nc21 ; CCn1nnc2ccc(-c3c(F)cn4nc(N[C@@H]5CCN(S(C)(=O)=O)CC5(F)F)nc(NC)c34)nc21] 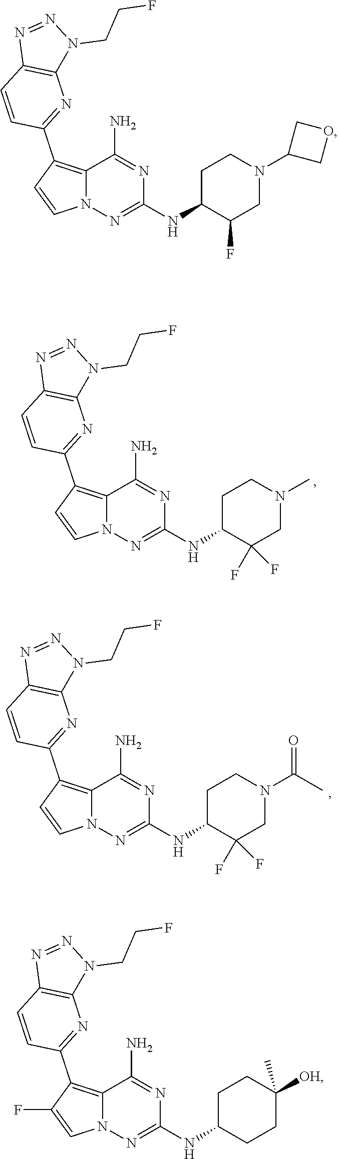 [SMILES: Nc1nc(N[C@H]2CCN(C3COC3)C[C@H]2F)nn2ccc(-c3ccc4nnn(CCF)c4n3)c12 ; CN1CC[C@@H](Nc2nc(N)c3c(-c4ccc5nnn(CCF)c5n4)ccn3n2)C(F)(F)C1 ; CC(=O)N1CC[C@@H](Nc2nc(N)c3c(-c4ccc5nnn(CCF)c5n4)ccn3n2)C(F)(F)C1 ; C[C@]1(O)CC[C@H](Nc2nc(N)c3c(-c4ccc5nnn(CCF)c5n4)c(F)cn3n2)CC1] 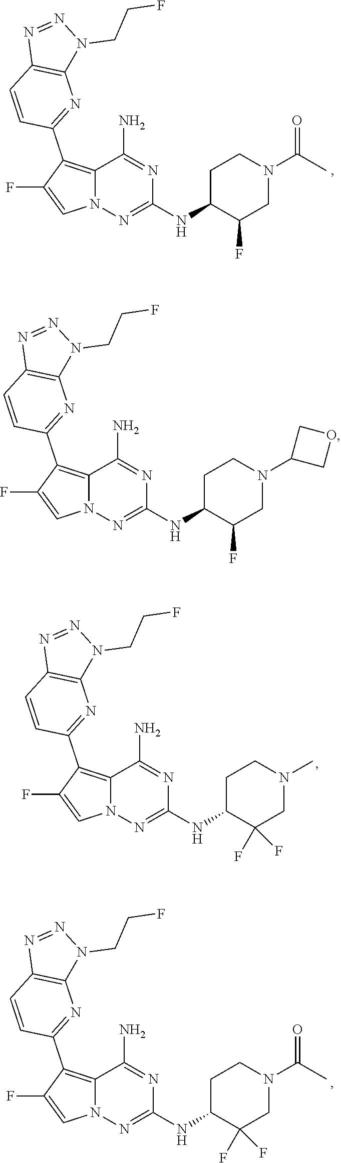 [SMILES: CC(=O)N1CC[C@H](Nc2nc(N)c3c(-c4ccc5nnn(CCF)c5n4)c(F)cn3n2)[C@H](F)C1 ; Nc1nc(N[C@H]2CCN(C3COC3)C[C@H]2F)nn2cc(F)c(-c3ccc4nnn(CCF)c4n3)c12 ; CN1CC[C@@H](Nc2nc(N)c3c(-c4ccc5nnn(CCF)c5n4)c(F)cn3n2)C(F)(F)C1 ; CC(=O)N1CC[C@@H](Nc2nc(N)c3c(-c4ccc5nnn(CCF)c5n4)c(F)cn3n2)C(F)(F)C1] 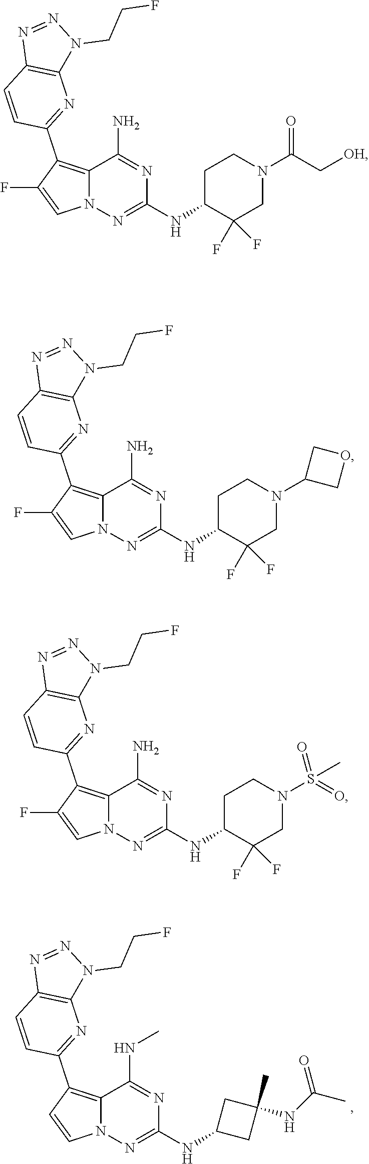 [SMILES: Nc1nc(N[C@@H]2CCN(C(=O)CO)CC2(F)F)nn2cc(F)c(-c3ccc4nnn(CCF)c4n3)c12 ; Nc1nc(N[C@@H]2CCN(C3COC3)CC2(F)F)nn2cc(F)c(-c3ccc4nnn(CCF)c4n3)c12 ; CS(=O)(=O)N1CC[C@@H](Nc2nc(N)c3c(-c4ccc5nnn(CCF)c5n4)c(F)cn3n2)C(F)(F)C1 ; CNc1nc(N[C@H]2C[C@@](C)(NC(C)=O)C2)nn2ccc(-c3ccc4nnn(CCF)c4n3)c12] 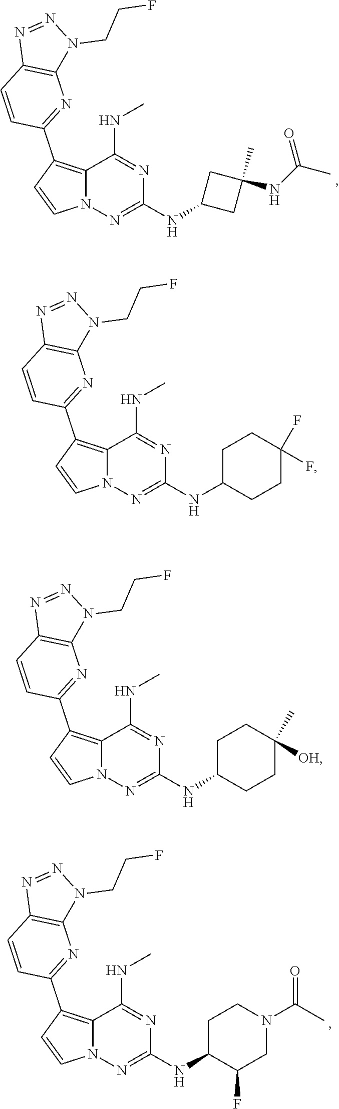 [SMILES: CNc1nc(N[C@H]2C[C@](C)(NC(C)=O)C2)nn2ccc(-c3ccc4nnn(CCF)c4n3)c12 ; CNc1nc(NC2CCC(F)(F)CC2)nn2ccc(-c3ccc4nnn(CCF)c4n3)c12 ; CNc1nc(N[C@H]2CC[C@](C)(O)CC2)nn2ccc(-c3ccc4nnn(CCF)c4n3)c12 ; CNc1nc(N[C@H]2CCN(C(C)=O)C[C@H]2F)nn2ccc(-c3ccc4nnn(CCF)c4n3)c12] 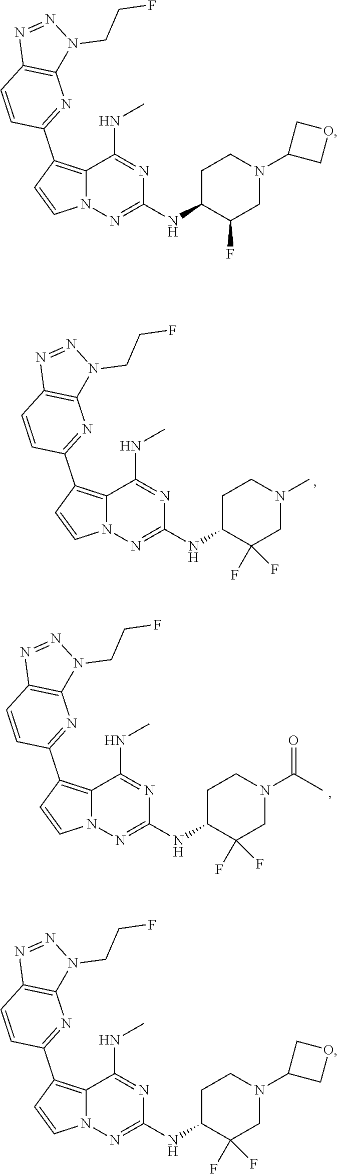 [SMILES: CNc1nc(N[C@H]2CCN(C3COC3)C[C@H]2F)nn2ccc(-c3ccc4nnn(CCF)c4n3)c12 ; CNc1nc(N[C@@H]2CCN(C)CC2(F)F)nn2ccc(-c3ccc4nnn(CCF)c4n3)c12 ; CNc1nc(N[C@@H]2CCN(C(C)=O)CC2(F)F)nn2ccc(-c3ccc4nnn(CCF)c4n3)c12 ; CNc1nc(N[C@@H]2CCN(C3COC3)CC2(F)F)nn2ccc(-c3ccc4nnn(CCF)c4n3)c12] 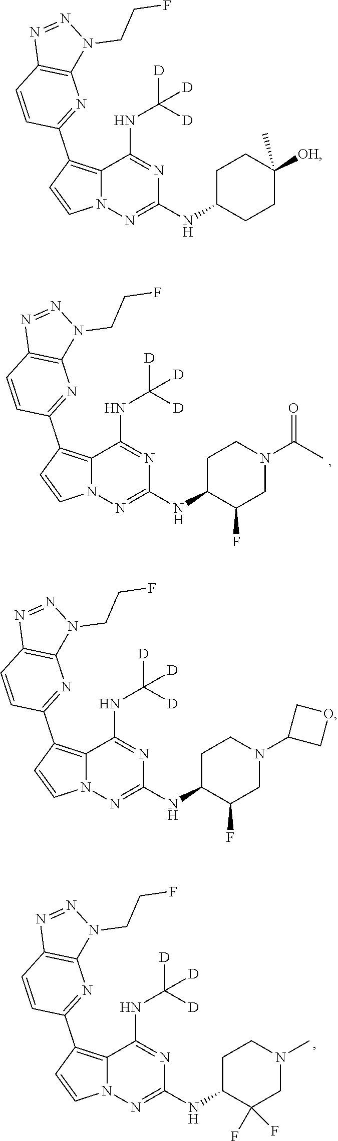 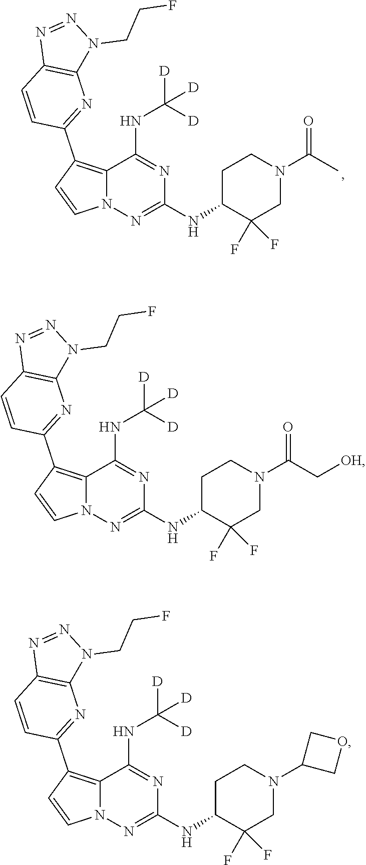 [SMILES: [2H]C([2H])([2H])Nc1nc(N[C@@H]2CCN(C(C)=O)CC2(F)F)nn2ccc(-c3ccc4nnn(CCF)c4n3)c12 ; [2H]C([2H])([2H])Nc1nc(N[C@@H]2CCN(C(=O)CO)CC2(F)F)nn2ccc(-c3ccc4nnn(CCF)c4n3)c12 ; [2H]C([2H])([2H])Nc1nc(N[C@@H]2CCN(C3COC3)CC2(F)F)nn2ccc(-c3ccc4nnn(CCF)c4n3)c12] 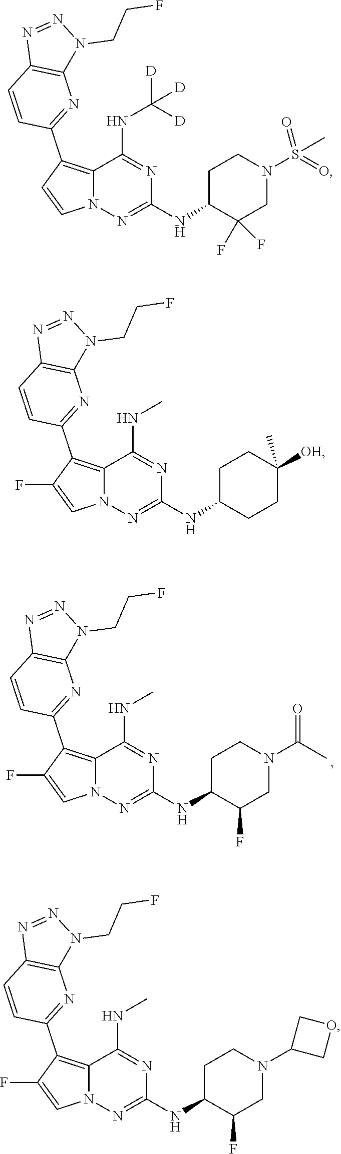 [SMILES: [2H]C([2H])([2H])Nc1nc(N[C@@H]2CCN(S(C)(=O)=O)CC2(F)F)nn2ccc(-c3ccc4nnn(CCF)c4n3)c12 ; CNc1nc(N[C@H]2CC[C@](C)(O)CC2)nn2cc(F)c(-c3ccc4nnn(CCF)c4n3)c12 ; CNc1nc(N[C@H]2CCN(C(C)=O)C[C@H]2F)nn2cc(F)c(-c3ccc4nnn(CCF)c4n3)c12 ; CNc1nc(N[C@H]2CCN(C3COC3)C[C@H]2F)nn2cc(F)c(-c3ccc4nnn(CCF)c4n3)c12] 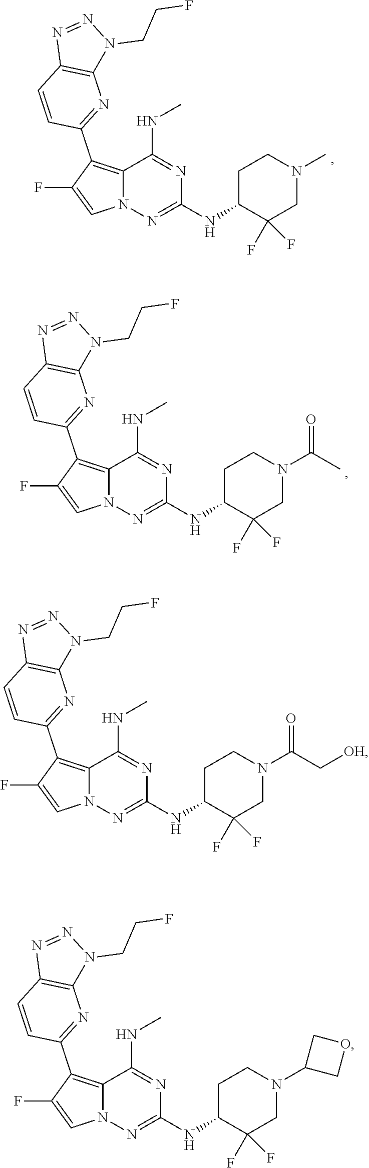 [SMILES: CNc1nc(N[C@@H]2CCN(C)CC2(F)F)nn2cc(F)c(-c3ccc4nnn(CCF)c4n3)c12 ; CNc1nc(N[C@@H]2CCN(C(C)=O)CC2(F)F)nn2cc(F)c(-c3ccc4nnn(CCF)c4n3)c12 ; CNc1nc(N[C@@H]2CCN(C(=O)CO)CC2(F)F)nn2cc(F)c(-c3ccc4nnn(CCF)c4n3)c12 ; CNc1nc(N[C@@H]2CCN(C3COC3)CC2(F)F)nn2cc(F)c(-c3ccc4nnn(CCF)c4n3)c12] 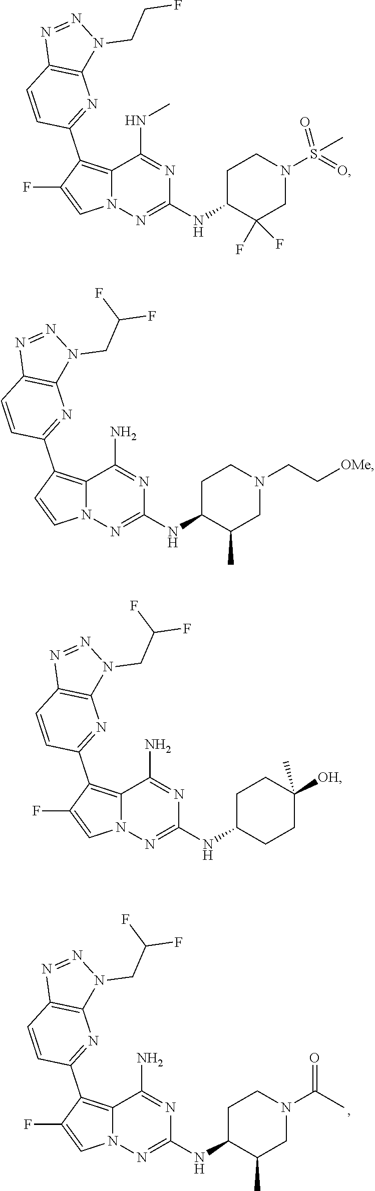 [SMILES: CNc1nc(N[C@@H]2CCN(S(C)(=O)=O)CC2(F)F)nn2cc(F)c(-c3ccc4nnn(CCF)c4n3)c12 ; COCCN1CC[C@H](Nc2nc(N)c3c(-c4ccc5nnn(CC(F)F)c5n4)ccn3n2)[C@H](C)C1 ; C[C@]1(O)CC[C@H](Nc2nc(N)c3c(-c4ccc5nnn(CC(F)F)c5n4)c(F)cn3n2)CC1 ; CC(=O)N1CC[C@H](Nc2nc(N)c3c(-c4ccc5nnn(CC(F)F)c5n4)c(F)cn3n2)[C@H](C)C1] 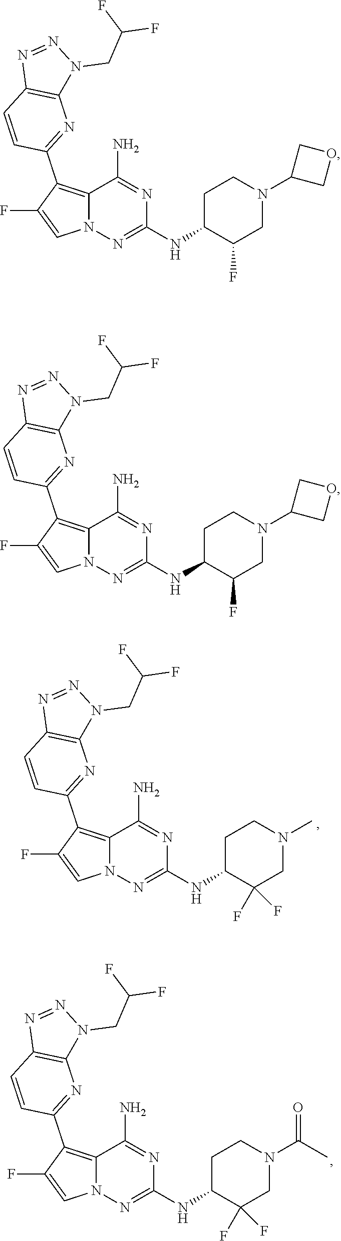 [SMILES: Nc1nc(N[C@@H]2CCN(C3COC3)C[C@@H]2F)nn2cc(F)c(-c3ccc4nnn(CC(F)F)c4n3)c12 ; Nc1nc(N[C@H]2CCN(C3COC3)C[C@H]2F)nn2cc(F)c(-c3ccc4nnn(CC(F)F)c4n3)c12 ; CN1CC[C@@H](Nc2nc(N)c3c(-c4ccc5nnn(CC(F)F)c5n4)c(F)cn3n2)C(F)(F)C1 ; CC(=O)N1CC[C@@H](Nc2nc(N)c3c(-c4ccc5nnn(CC(F)F)c5n4)c(F)cn3n2)C(F)(F)C1] 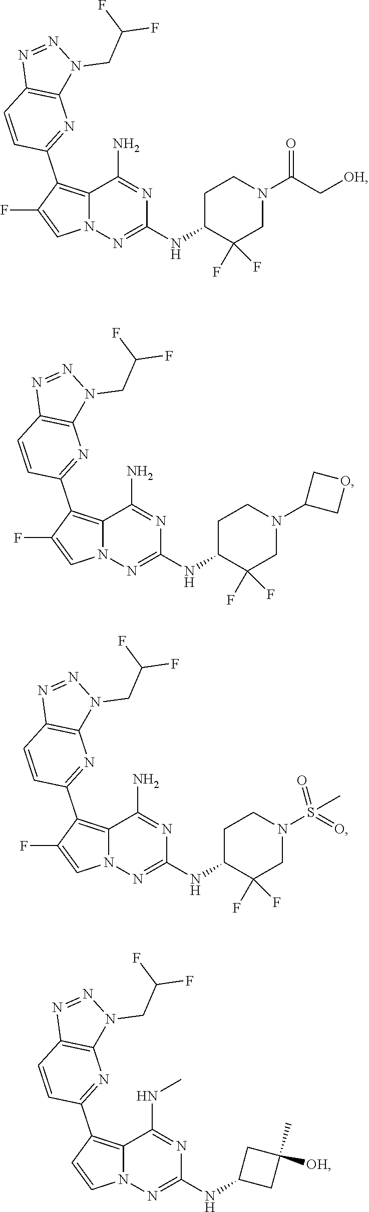 [SMILES: Nc1nc(N[C@@H]2CCN(C(=O)CO)CC2(F)F)nn2cc(F)c(-c3ccc4nnn(CC(F)F)c4n3)c12 ; Nc1nc(N[C@@H]2CCN(C3COC3)CC2(F)F)nn2cc(F)c(-c3ccc4nnn(CC(F)F)c4n3)c12 ; CS(=O)(=O)N1CC[C@@H](Nc2nc(N)c3c(-c4ccc5nnn(CC(F)F)c5n4)c(F)cn3n2)C(F)(F)C1 ; CNc1nc(N[C@H]2C[C@](C)(O)C2)nn2ccc(-c3ccc4nnn(CC(F)F)c4n3)c12] 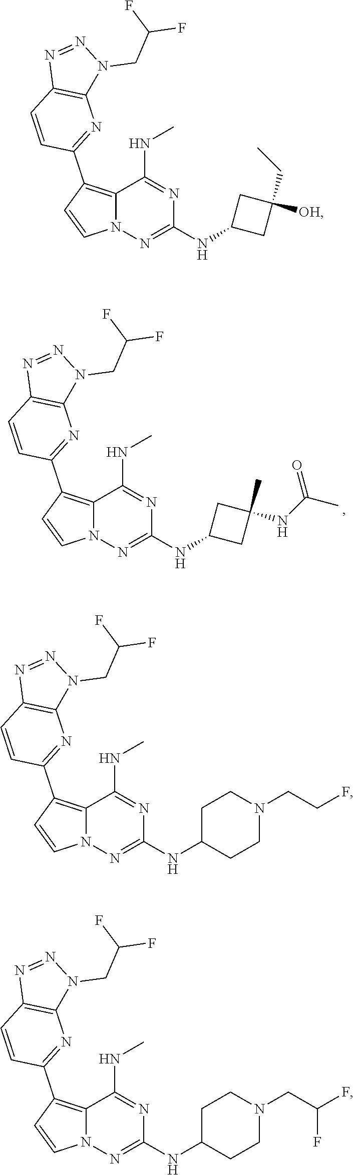 [SMILES: CC[C@]1(O)C[C@H](Nc2nc(NC)c3c(-c4ccc5nnn(CC(F)F)c5n4)ccn3n2)C1 ; CNc1nc(N[C@H]2C[C@@](C)(NC(C)=O)C2)nn2ccc(-c3ccc4nnn(CC(F)F)c4n3)c12 ; CNc1nc(NC2CCN(CCF)CC2)nn2ccc(-c3ccc4nnn(CC(F)F)c4n3)c12 ; CNc1nc(NC2CCN(CC(F)F)CC2)nn2ccc(-c3ccc4nnn(CC(F)F)c4n3)c12] 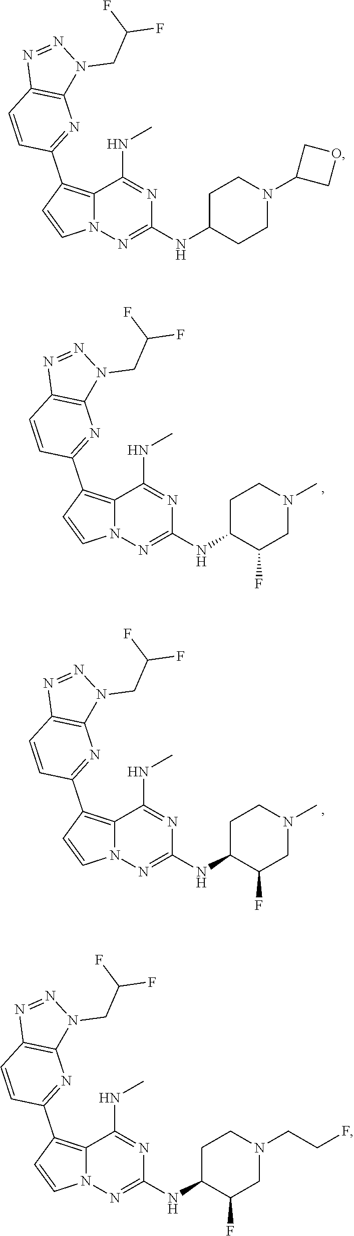 [SMILES: CNc1nc(NC2CCN(C3COC3)CC2)nn2ccc(-c3ccc4nnn(CC(F)F)c4n3)c12 ; CNc1nc(N[C@@H]2CCN(C)C[C@@H]2F)nn2ccc(-c3ccc4nnn(CC(F)F)c4n3)c12 ; CNc1nc(N[C@H]2CCN(C)C[C@H]2F)nn2ccc(-c3ccc4nnn(CC(F)F)c4n3)c12 ; CNc1nc(N[C@H]2CCN(CCF)C[C@H]2F)nn2ccc(-c3ccc4nnn(CC(F)F)c4n3)c12] 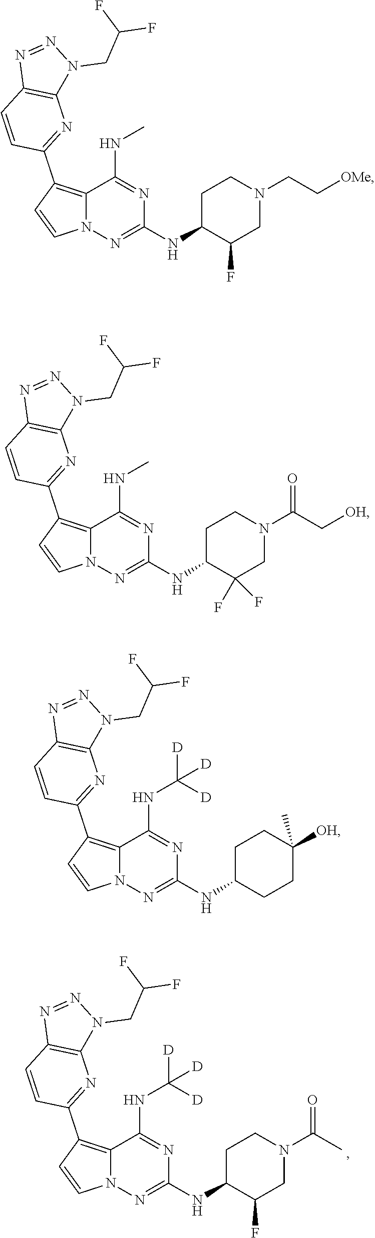 [SMILES: CNc1nc(N[C@H]2CCN(CCOC)C[C@H]2F)nn2ccc(-c3ccc4nnn(CC(F)F)c4n3)c12 ; CNc1nc(N[C@@H]2CCN(C(=O)CO)CC2(F)F)nn2ccc(-c3ccc4nnn(CC(F)F)c4n3)c12 ; [2H]C([2H])([2H])Nc1nc(N[C@H]2CC[C@](C)(O)CC2)nn2ccc(-c3ccc4nnn(CC(F)F)c4n3)c12 ; [2H]C([2H])([2H])Nc1nc(N[C@H]2CCN(C(C)=O)C[C@H]2F)nn2ccc(-c3ccc4nnn(CC(F)F)c4n3)c12] 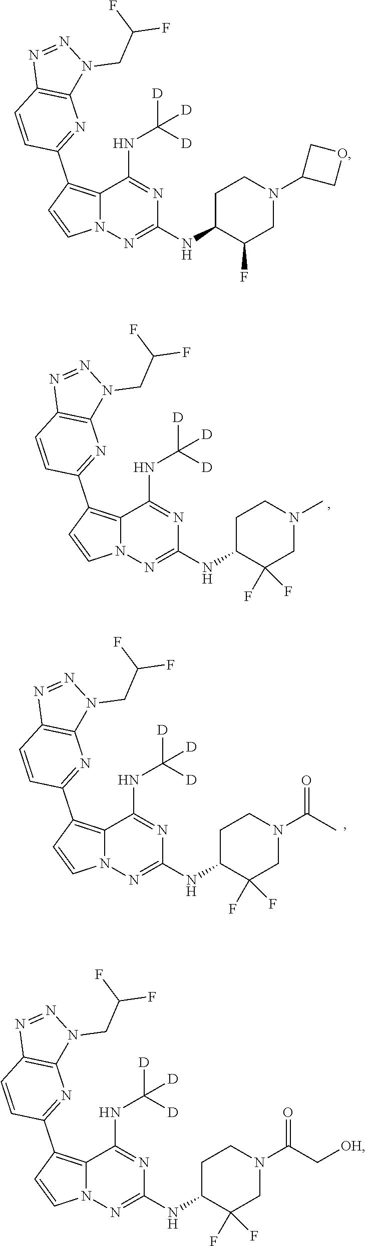 [SMILES: [2H]C([2H])([2H])Nc1nc(N[C@H]2CCN(C3COC3)C[C@H]2F)nn2ccc(-c3ccc4nnn(CC(F)F)c4n3)c12 ; [2H]C([2H])([2H])Nc1nc(N[C@@H]2CCN(C)CC2(F)F)nn2ccc(-c3ccc4nnn(CC(F)F)c4n3)c12 ; [2H]C([2H])([2H])Nc1nc(N[C@@H]2CCN(C(C)=O)CC2(F)F)nn2ccc(-c3ccc4nnn(CC(F)F)c4n3)c12 ; [2H]C([2H])([2H])Nc1nc(N[C@@H]2CCN(C(=O)CO)CC2(F)F)nn2ccc(-c3ccc4nnn(CC(F)F)c4n3)c12] 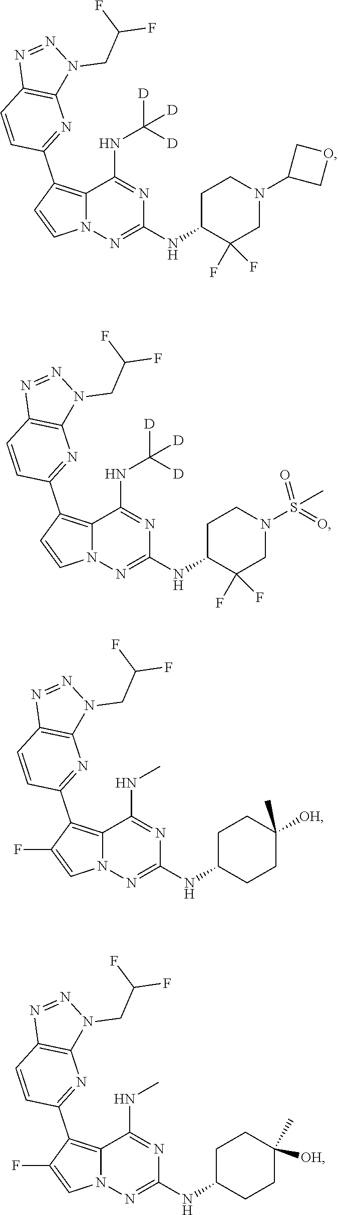 [SMILES: [2H]C([2H])([2H])Nc1nc(N[C@@H]2CCN(C3COC3)CC2(F)F)nn2ccc(-c3ccc4nnn(CC(F)F)c4n3)c12 ; [2H]C([2H])([2H])Nc1nc(N[C@@H]2CCN(S(C)(=O)=O)CC2(F)F)nn2ccc(-c3ccc4nnn(CC(F)F)c4n3)c12 ; CNc1nc(N[C@H]2CC[C@@](C)(O)CC2)nn2cc(F)c(-c3ccc4nnn(CC(F)F)c4n3)c12 ; CNc1nc(N[C@H]2CC[C@](C)(O)CC2)nn2cc(F)c(-c3ccc4nnn(CC(F)F)c4n3)c12] 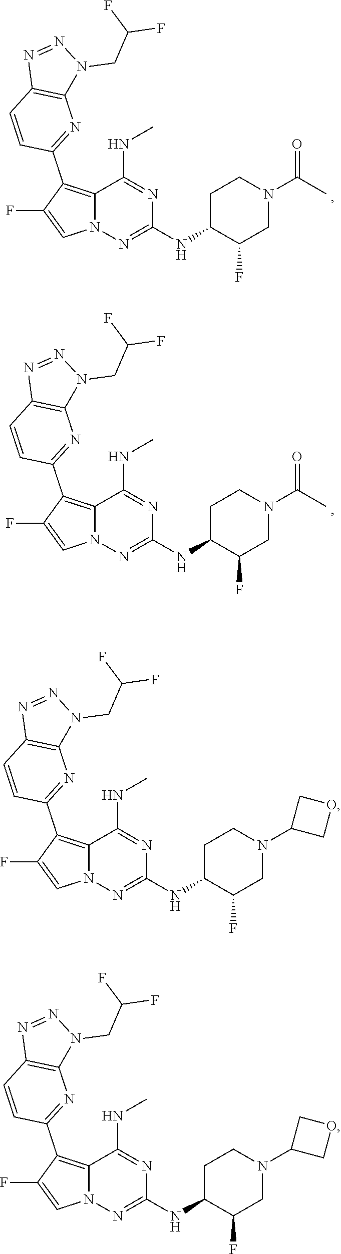 [SMILES: CNc1nc(N[C@@H]2CCN(C(C)=O)C[C@@H]2F)nn2cc(F)c(-c3ccc4nnn(CC(F)F)c4n3)c12 ; CNc1nc(N[C@H]2CCN(C(C)=O)C[C@H]2F)nn2cc(F)c(-c3ccc4nnn(CC(F)F)c4n3)c12 ; CNc1nc(N[C@@H]2CCN(C3COC3)C[C@@H]2F)nn2cc(F)c(-c3ccc4nnn(CC(F)F)c4n3)c12 ; CNc1nc(N[C@H]2CCN(C3COC3)C[C@H]2F)nn2cc(F)c(-c3ccc4nnn(CC(F)F)c4n3)c12] 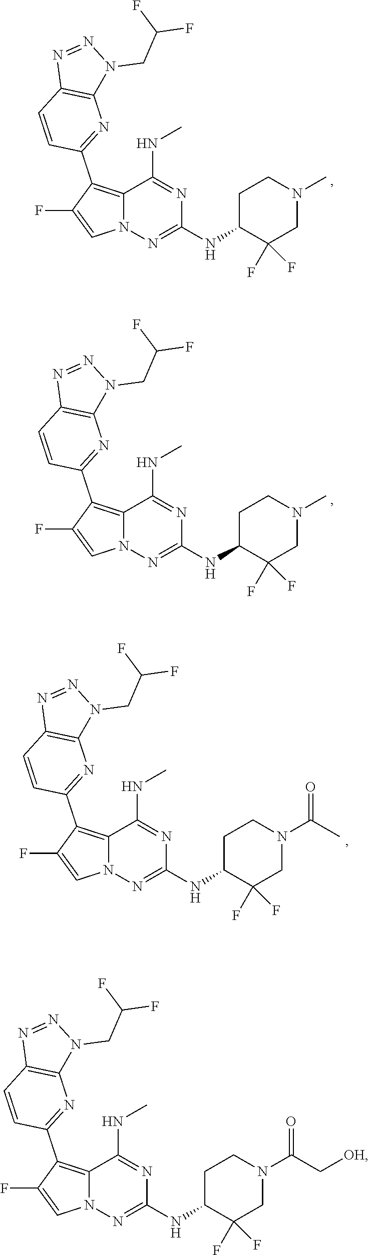 [SMILES: CNc1nc(N[C@@H]2CCN(C)CC2(F)F)nn2cc(F)c(-c3ccc4nnn(CC(F)F)c4n3)c12 ; CNc1nc(N[C@H]2CCN(C)CC2(F)F)nn2cc(F)c(-c3ccc4nnn(CC(F)F)c4n3)c12 ; CNc1nc(N[C@@H]2CCN(C(C)=O)CC2(F)F)nn2cc(F)c(-c3ccc4nnn(CC(F)F)c4n3)c12 ; CNc1nc(N[C@@H]2CCN(C(=O)CO)CC2(F)F)nn2cc(F)c(-c3ccc4nnn(CC(F)F)c4n3)c12] 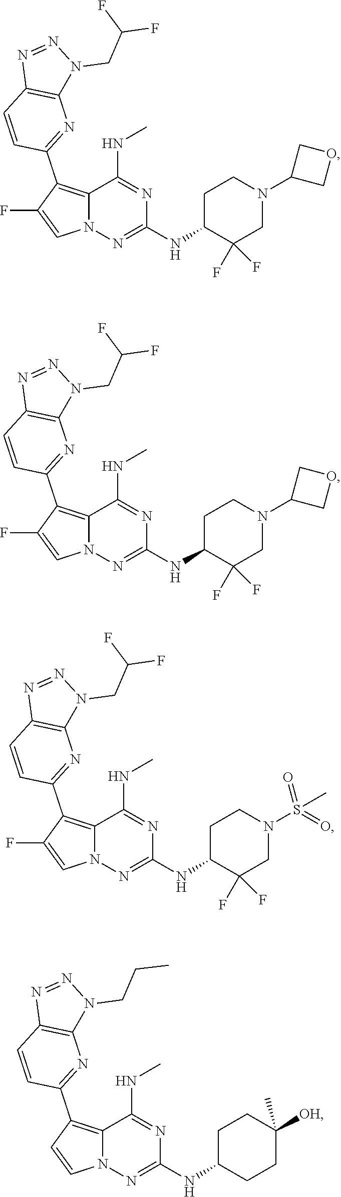 [SMILES: CNc1nc(N[C@@H]2CCN(C3COC3)CC2(F)F)nn2cc(F)c(-c3ccc4nnn(CC(F)F)c4n3)c12 ; CNc1nc(N[C@H]2CCN(C3COC3)CC2(F)F)nn2cc(F)c(-c3ccc4nnn(CC(F)F)c4n3)c12 ; CNc1nc(N[C@@H]2CCN(S(C)(=O)=O)CC2(F)F)nn2cc(F)c(-c3ccc4nnn(CC(F)F)c4n3)c12 ; CCCn1nnc2ccc(-c3ccn4nc(N[C@H]5CC[C@](C)(O)CC5)nc(NC)c34)nc21] 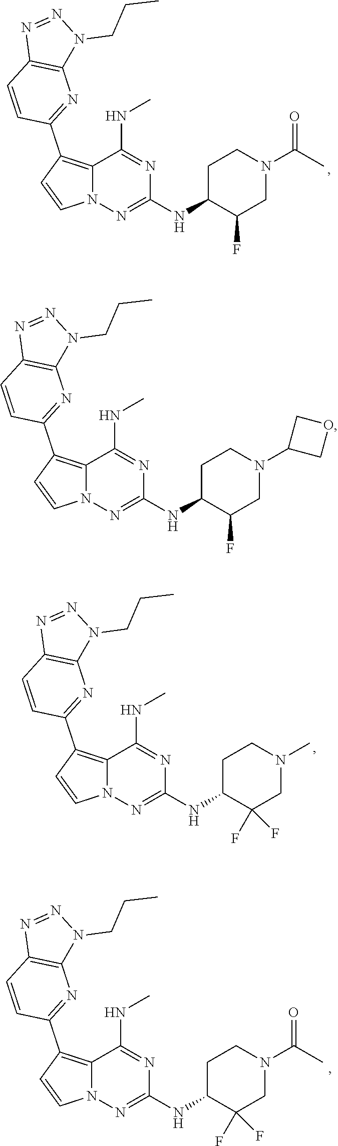 [SMILES: CCCn1nnc2ccc(-c3ccn4nc(N[C@H]5CCN(C(C)=O)C[C@H]5F)nc(NC)c34)nc21 ; CCCn1nnc2ccc(-c3ccn4nc(N[C@H]5CCN(C6COC6)C[C@H]5F)nc(NC)c34)nc21 ; CCCn1nnc2ccc(-c3ccn4nc(N[C@@H]5CCN(C)CC5(F)F)nc(NC)c34)nc21 ; CCCn1nnc2ccc(-c3ccn4nc(N[C@@H]5CCN(C(C)=O)CC5(F)F)nc(NC)c34)nc21] 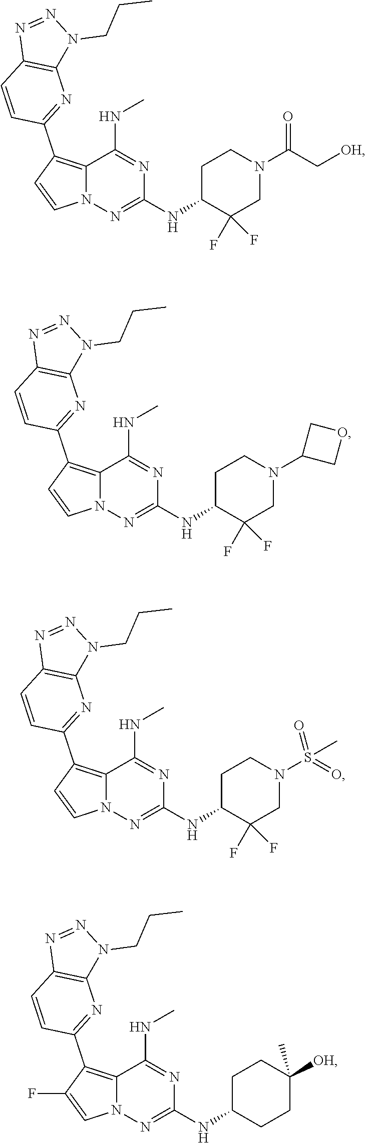 [SMILES: CCCn1nnc2ccc(-c3ccn4nc(N[C@@H]5CCN(C(=O)CO)CC5(F)F)nc(NC)c34)nc21 ; CCCn1nnc2ccc(-c3ccn4nc(N[C@@H]5CCN(C6COC6)CC5(F)F)nc(NC)c34)nc21 ; CCCn1nnc2ccc(-c3ccn4nc(N[C@@H]5CCN(S(C)(=O)=O)CC5(F)F)nc(NC)c34)nc21 ; CCCn1nnc2ccc(-c3c(F)cn4nc(N[C@H]5CC[C@](C)(O)CC5)nc(NC)c34)nc21] 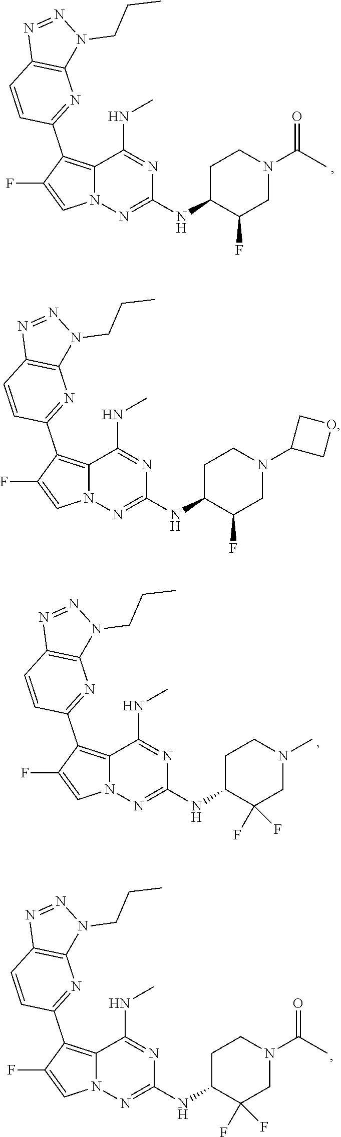 [SMILES: CCCn1nnc2ccc(-c3c(F)cn4nc(N[C@H]5CCN(C(C)=O)C[C@H]5F)nc(NC)c34)nc21 ; CCCn1nnc2ccc(-c3c(F)cn4nc(N[C@H]5CCN(C6COC6)C[C@H]5F)nc(NC)c34)nc21 ; CCCn1nnc2ccc(-c3c(F)cn4nc(N[C@@H]5CCN(C)CC5(F)F)nc(NC)c34)nc21 ; CCCn1nnc2ccc(-c3c(F)cn4nc(N[C@@H]5CCN(C(C)=O)CC5(F)F)nc(NC)c34)nc21] 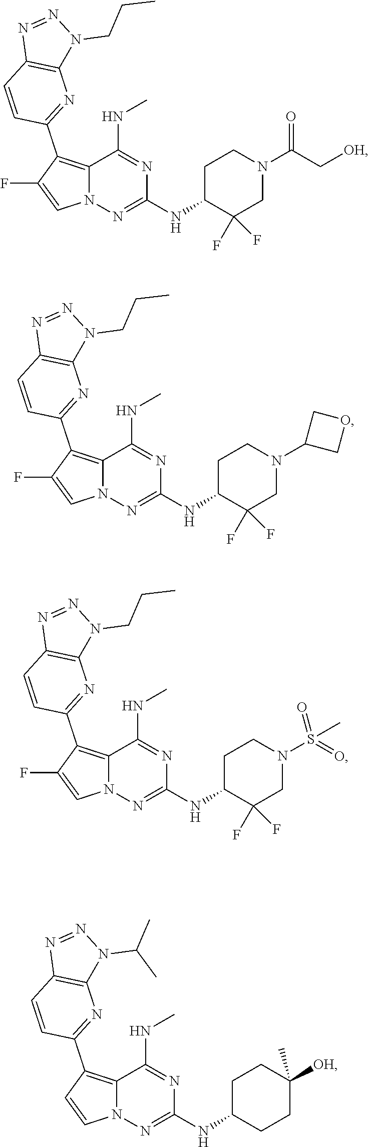 [SMILES: CCCn1nnc2ccc(-c3c(F)cn4nc(N[C@@H]5CCN(C(=O)CO)CC5(F)F)nc(NC)c34)nc21 ; CCCn1nnc2ccc(-c3c(F)cn4nc(N[C@@H]5CCN(C6COC6)CC5(F)F)nc(NC)c34)nc21 ; CCCn1nnc2ccc(-c3c(F)cn4nc(N[C@@H]5CCN(S(C)(=O)=O)CC5(F)F)nc(NC)c34)nc21 ; CNc1nc(N[C@H]2CC[C@](C)(O)CC2)nn2ccc(-c3ccc4nnn(C(C)C)c4n3)c12] 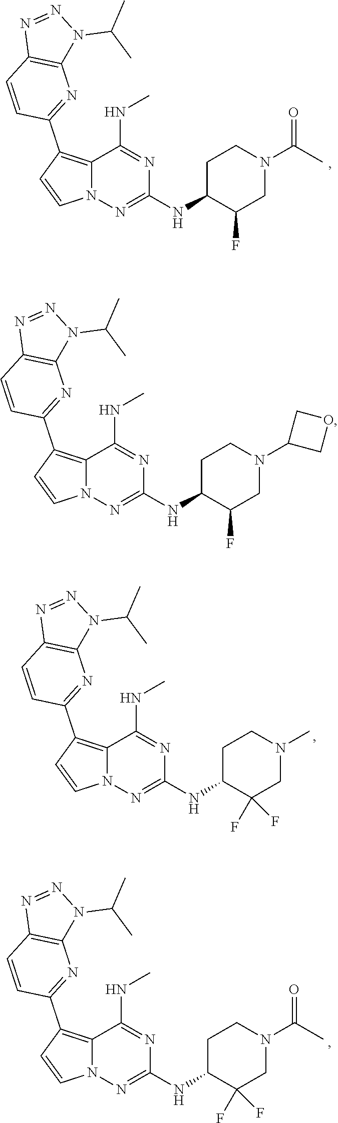 [SMILES: CNc1nc(N[C@H]2CCN(C(C)=O)C[C@H]2F)nn2ccc(-c3ccc4nnn(C(C)C)c4n3)c12 ; CNc1nc(N[C@H]2CCN(C3COC3)C[C@H]2F)nn2ccc(-c3ccc4nnn(C(C)C)c4n3)c12 ; CNc1nc(N[C@@H]2CCN(C)CC2(F)F)nn2ccc(-c3ccc4nnn(C(C)C)c4n3)c12 ; CNc1nc(N[C@@H]2CCN(C(C)=O)CC2(F)F)nn2ccc(-c3ccc4nnn(C(C)C)c4n3)c12] 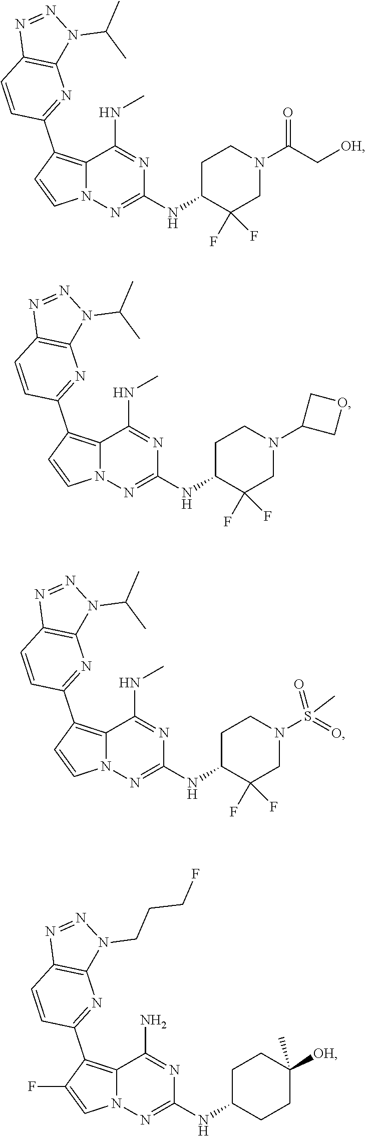 [SMILES: CNc1nc(N[C@@H]2CCN(C(=O)CO)CC2(F)F)nn2ccc(-c3ccc4nnn(C(C)C)c4n3)c12 ; CNc1nc(N[C@@H]2CCN(C3COC3)CC2(F)F)nn2ccc(-c3ccc4nnn(C(C)C)c4n3)c12 ; CNc1nc(N[C@@H]2CCN(S(C)(=O)=O)CC2(F)F)nn2ccc(-c3ccc4nnn(C(C)C)c4n3)c12 ; C[C@]1(O)CC[C@H](Nc2nc(N)c3c(-c4ccc5nnn(CCCF)c5n4)c(F)cn3n2)CC1] 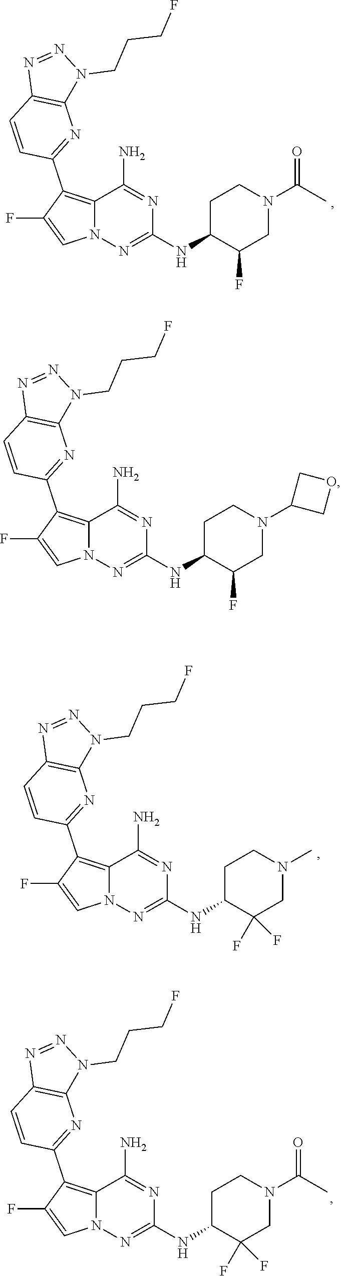 [SMILES: CC(=O)N1CC[C@H](Nc2nc(N)c3c(-c4ccc5nnn(CCCF)c5n4)c(F)cn3n2)[C@H](F)C1 ; Nc1nc(N[C@H]2CCN(C3COC3)C[C@H]2F)nn2cc(F)c(-c3ccc4nnn(CCCF)c4n3)c12 ; CN1CC[C@@H](Nc2nc(N)c3c(-c4ccc5nnn(CCCF)c5n4)c(F)cn3n2)C(F)(F)C1 ; CC(=O)N1CC[C@@H](Nc2nc(N)c3c(-c4ccc5nnn(CCCF)c5n4)c(F)cn3n2)C(F)(F)C1] 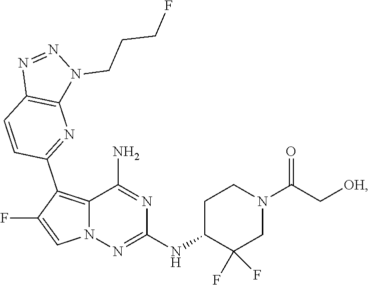 [SMILES: Nc1nc(N[C@@H]2CCN(C(=O)CO)CC2(F)F)nn2cc(F)c(-c3ccc4nnn(CCCF)c4n3)c12] 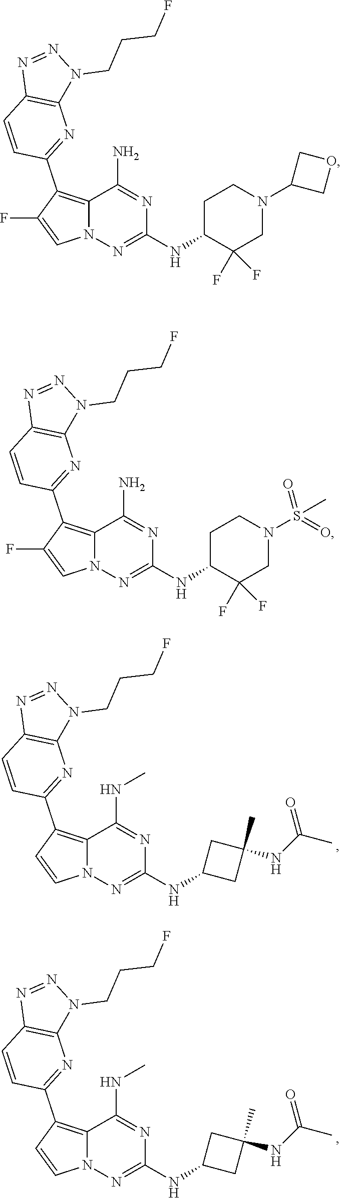 [SMILES: Nc1nc(N[C@@H]2CCN(C3COC3)CC2(F)F)nn2cc(F)c(-c3ccc4nnn(CCCF)c4n3)c12 ; CS(=O)(=O)N1CC[C@@H](Nc2nc(N)c3c(-c4ccc5nnn(CCCF)c5n4)c(F)cn3n2)C(F)(F)C1 ; CNc1nc(N[C@H]2C[C@@](C)(NC(C)=O)C2)nn2ccc(-c3ccc4nnn(CCCF)c4n3)c12 ; CNc1nc(N[C@H]2C[C@](C)(NC(C)=O)C2)nn2ccc(-c3ccc4nnn(CCCF)c4n3)c12] 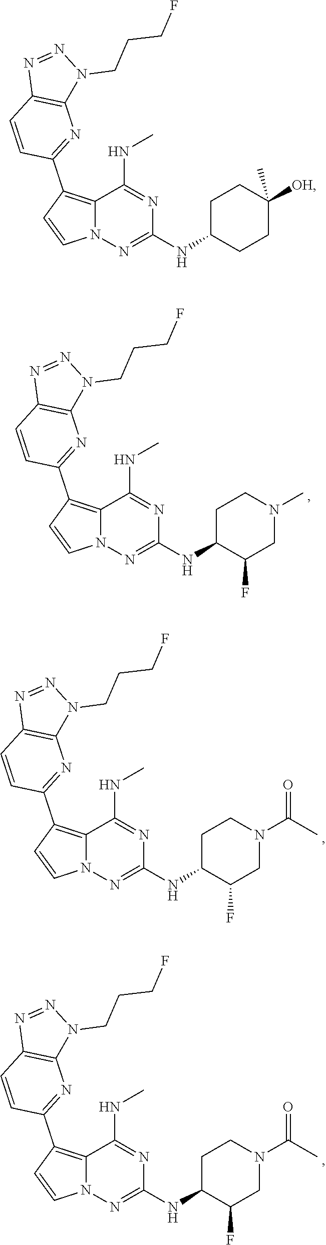 [SMILES: CNc1nc(N[C@H]2CC[C@](C)(O)CC2)nn2ccc(-c3ccc4nnn(CCCF)c4n3)c12 ; CNc1nc(N[C@H]2CCN(C)C[C@H]2F)nn2ccc(-c3ccc4nnn(CCCF)c4n3)c12 ; CNc1nc(N[C@@H]2CCN(C(C)=O)C[C@@H]2F)nn2ccc(-c3ccc4nnn(CCCF)c4n3)c12 ; CNc1nc(N[C@H]2CCN(C(C)=O)C[C@H]2F)nn2ccc(-c3ccc4nnn(CCCF)c4n3)c12] 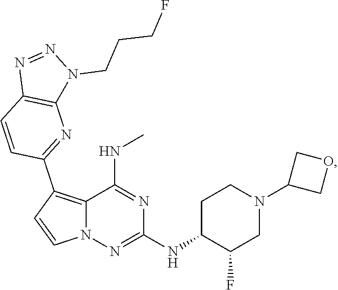 [SMILES: CNc1nc(N[C@@H]2CCN(C3COC3)C[C@@H]2F)nn2ccc(-c3ccc4nnn(CCCF)c4n3)c12] 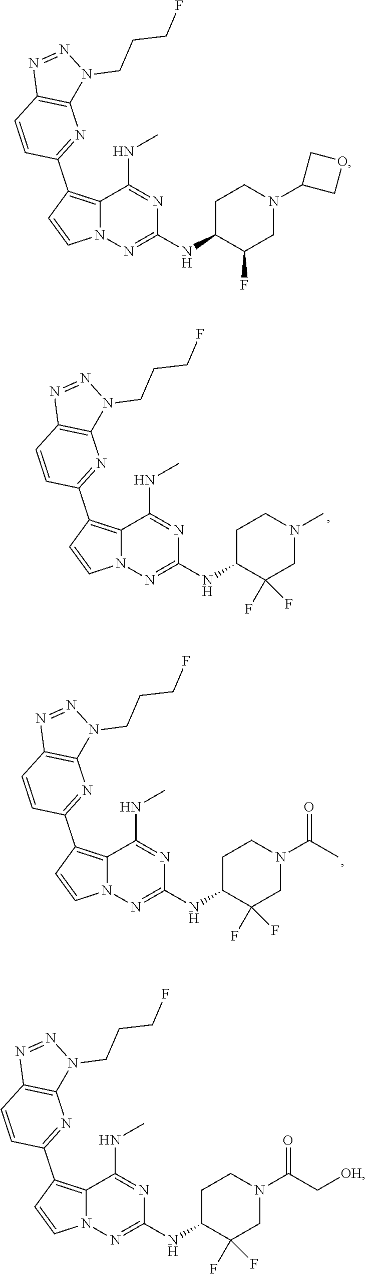 [SMILES: CNc1nc(N[C@H]2CCN(C3COC3)C[C@H]2F)nn2ccc(-c3ccc4nnn(CCCF)c4n3)c12 ; CNc1nc(N[C@@H]2CCN(C)CC2(F)F)nn2ccc(-c3ccc4nnn(CCCF)c4n3)c12 ; CNc1nc(N[C@@H]2CCN(C(C)=O)CC2(F)F)nn2ccc(-c3ccc4nnn(CCCF)c4n3)c12 ; CNc1nc(N[C@@H]2CCN(C(=O)CO)CC2(F)F)nn2ccc(-c3ccc4nnn(CCCF)c4n3)c12] 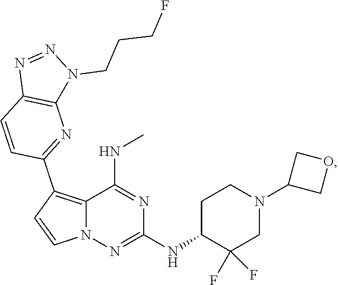 [SMILES: CNc1nc(N[C@@H]2CCN(C3COC3)CC2(F)F)nn2ccc(-c3ccc4nnn(CCCF)c4n3)c12] 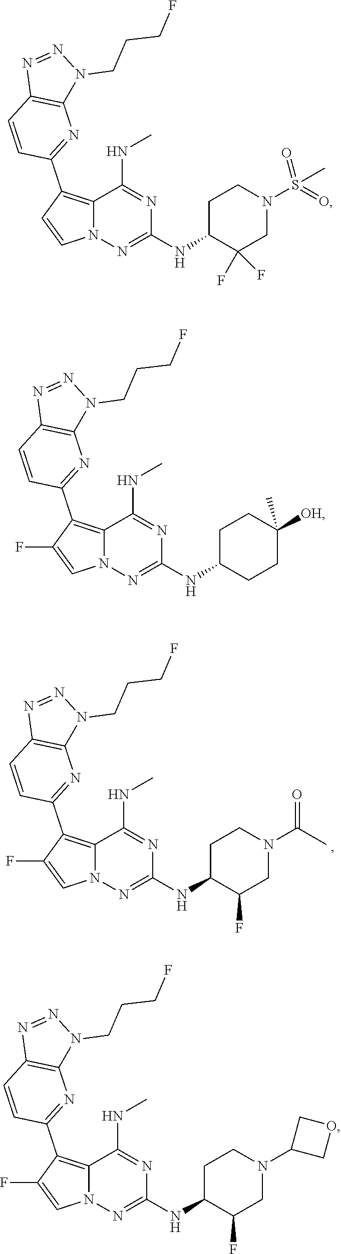 [SMILES: CNc1nc(N[C@@H]2CCN(S(C)(=O)=O)CC2(F)F)nn2ccc(-c3ccc4nnn(CCCF)c4n3)c12 ; CNc1nc(N[C@H]2CC[C@](C)(O)CC2)nn2cc(F)c(-c3ccc4nnn(CCCF)c4n3)c12 ; CNc1nc(N[C@H]2CCN(C(C)=O)C[C@H]2F)nn2cc(F)c(-c3ccc4nnn(CCCF)c4n3)c12 ; CNc1nc(N[C@H]2CCN(C3COC3)C[C@H]2F)nn2cc(F)c(-c3ccc4nnn(CCCF)c4n3)c12] 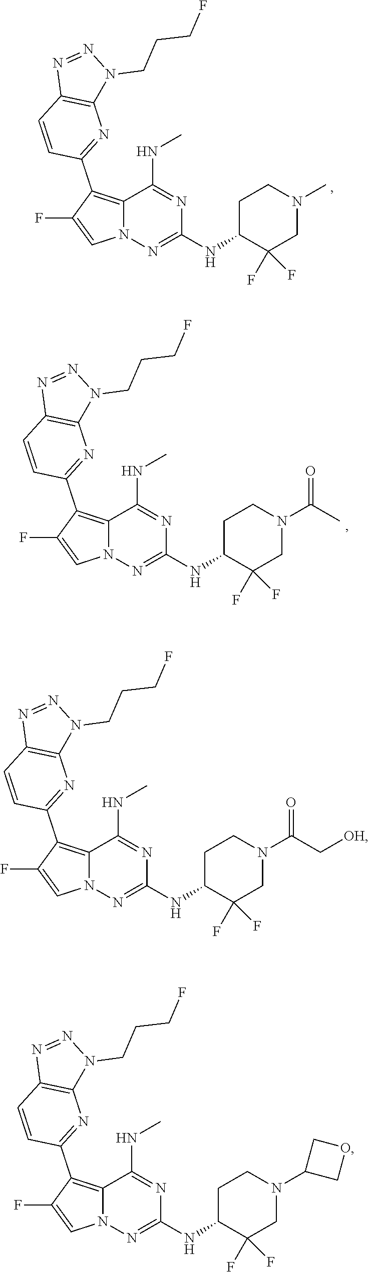 [SMILES: CNc1nc(N[C@@H]2CCN(C)CC2(F)F)nn2cc(F)c(-c3ccc4nnn(CCCF)c4n3)c12 ; CNc1nc(N[C@@H]2CCN(C(C)=O)CC2(F)F)nn2cc(F)c(-c3ccc4nnn(CCCF)c4n3)c12 ; CNc1nc(N[C@@H]2CCN(C(=O)CO)CC2(F)F)nn2cc(F)c(-c3ccc4nnn(CCCF)c4n3)c12 ; CNc1nc(N[C@@H]2CCN(C3COC3)CC2(F)F)nn2cc(F)c(-c3ccc4nnn(CCCF)c4n3)c12] 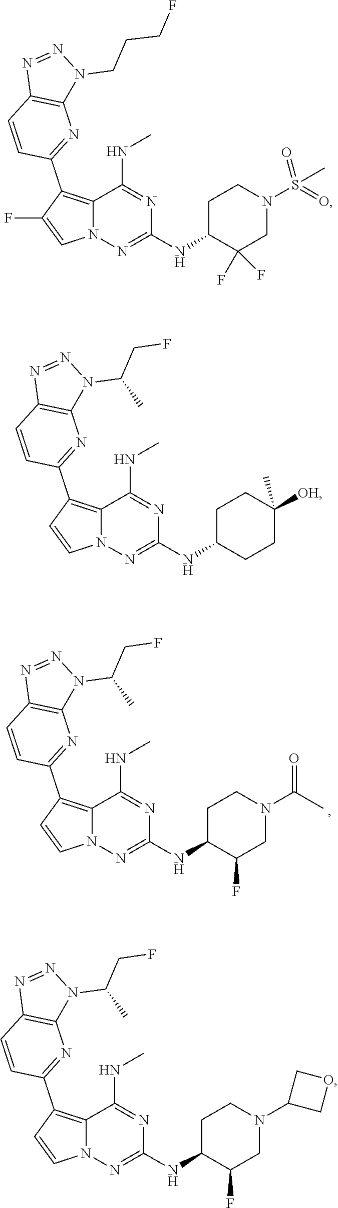 [SMILES: CNc1nc(N[C@@H]2CCN(S(C)(=O)=O)CC2(F)F)nn2cc(F)c(-c3ccc4nnn(CCCF)c4n3)c12 ; CNc1nc(N[C@H]2CC[C@](C)(O)CC2)nn2ccc(-c3ccc4nnn([C@@H](C)CF)c4n3)c12 ; CNc1nc(N[C@H]2CCN(C(C)=O)C[C@H]2F)nn2ccc(-c3ccc4nnn([C@@H](C)CF)c4n3)c12 ; CNc1nc(N[C@H]2CCN(C3COC3)C[C@H]2F)nn2ccc(-c3ccc4nnn([C@@H](C)CF)c4n3)c12] 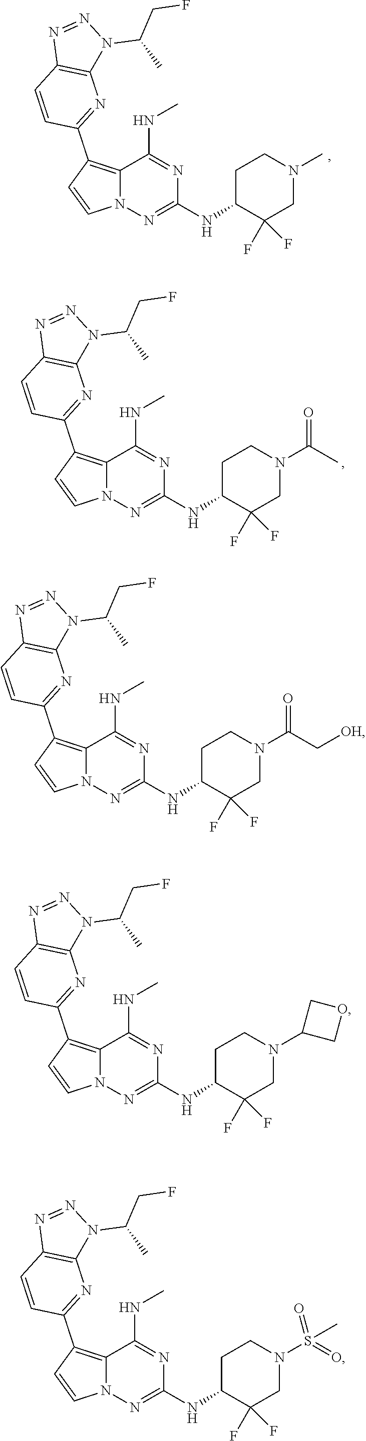 [SMILES: CNc1nc(N[C@@H]2CCN(C)CC2(F)F)nn2ccc(-c3ccc4nnn([C@@H](C)CF)c4n3)c12 ; CNc1nc(N[C@@H]2CCN(C(C)=O)CC2(F)F)nn2ccc(-c3ccc4nnn([C@@H](C)CF)c4n3)c12 ; CNc1nc(N[C@@H]2CCN(C(=O)CO)CC2(F)F)nn2ccc(-c3ccc4nnn([C@@H](C)CF)c4n3)c12 ; CNc1nc(N[C@@H]2CCN(C3COC3)CC2(F)F)nn2ccc(-c3ccc4nnn([C@@H](C)CF)c4n3)c12 ; CNc1nc(N[C@@H]2CCN(S(C)(=O)=O)CC2(F)F)nn2ccc(-c3ccc4nnn([C@@H](C)CF)c4n3)c12] 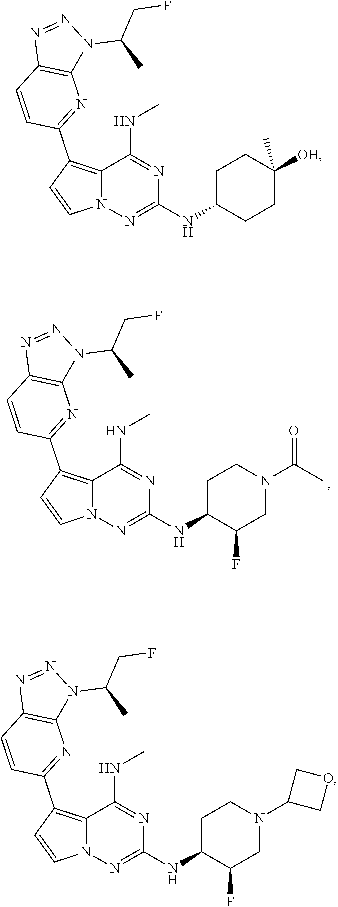 [SMILES: CNc1nc(N[C@H]2CC[C@](C)(O)CC2)nn2ccc(-c3ccc4nnn([C@H](C)CF)c4n3)c12 ; CNc1nc(N[C@H]2CCN(C(C)=O)C[C@H]2F)nn2ccc(-c3ccc4nnn([C@H](C)CF)c4n3)c12 ; CNc1nc(N[C@H]2CCN(C3COC3)C[C@H]2F)nn2ccc(-c3ccc4nnn([C@H](C)CF)c4n3)c12] 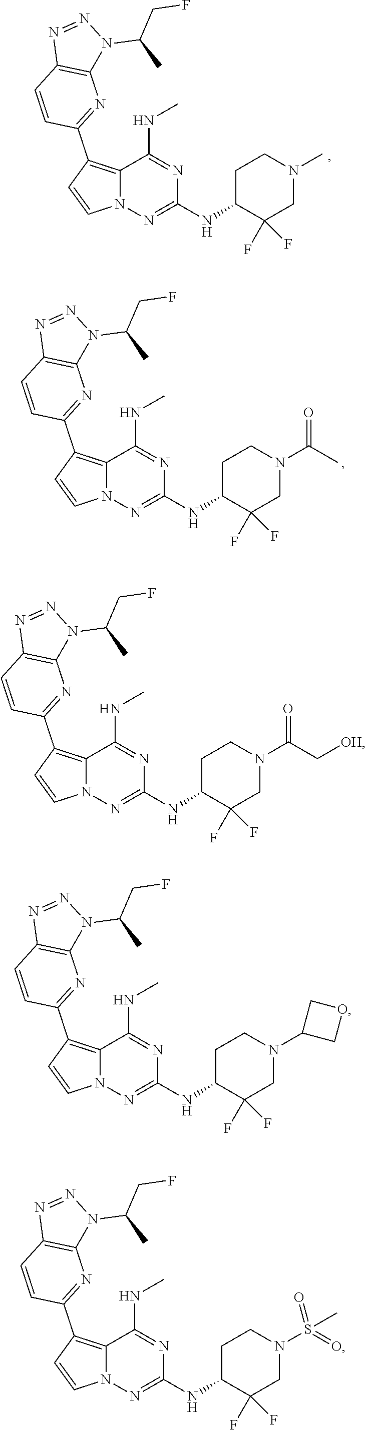 [SMILES: CNc1nc(N[C@@H]2CCN(C)CC2(F)F)nn2ccc(-c3ccc4nnn([C@H](C)CF)c4n3)c12 ; CNc1nc(N[C@@H]2CCN(C(C)=O)CC2(F)F)nn2ccc(-c3ccc4nnn([C@H](C)CF)c4n3)c12 ; CNc1nc(N[C@@H]2CCN(C(=O)CO)CC2(F)F)nn2ccc(-c3ccc4nnn([C@H](C)CF)c4n3)c12 ; CNc1nc(N[C@@H]2CCN(C3COC3)CC2(F)F)nn2ccc(-c3ccc4nnn([C@H](C)CF)c4n3)c12 ; CNc1nc(N[C@@H]2CCN(S(C)(=O)=O)CC2(F)F)nn2ccc(-c3ccc4nnn([C@H](C)CF)c4n3)c12] 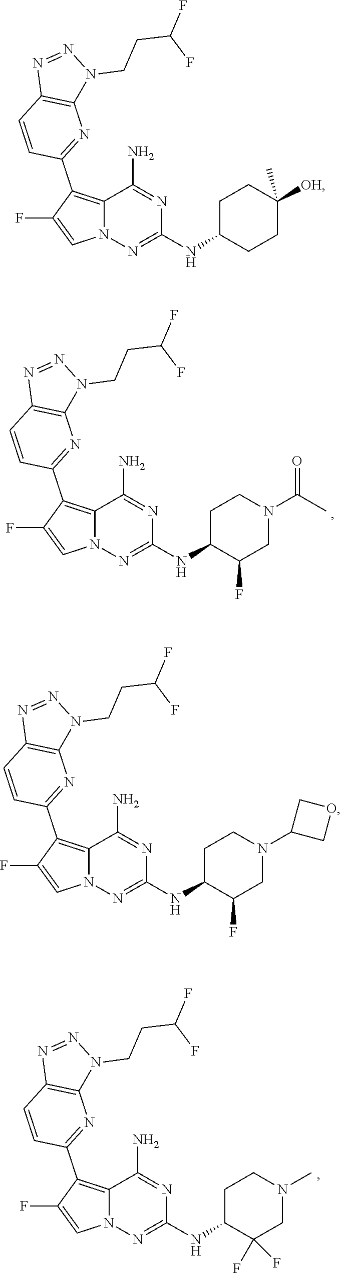 [SMILES: C[C@]1(O)CC[C@H](Nc2nc(N)c3c(-c4ccc5nnn(CCC(F)F)c5n4)c(F)cn3n2)CC1 ; CC(=O)N1CC[C@H](Nc2nc(N)c3c(-c4ccc5nnn(CCC(F)F)c5n4)c(F)cn3n2)[C@H](F)C1 ; Nc1nc(N[C@H]2CCN(C3COC3)C[C@H]2F)nn2cc(F)c(-c3ccc4nnn(CCC(F)F)c4n3)c12 ; CN1CC[C@@H](Nc2nc(N)c3c(-c4ccc5nnn(CCC(F)F)c5n4)c(F)cn3n2)C(F)(F)C1] 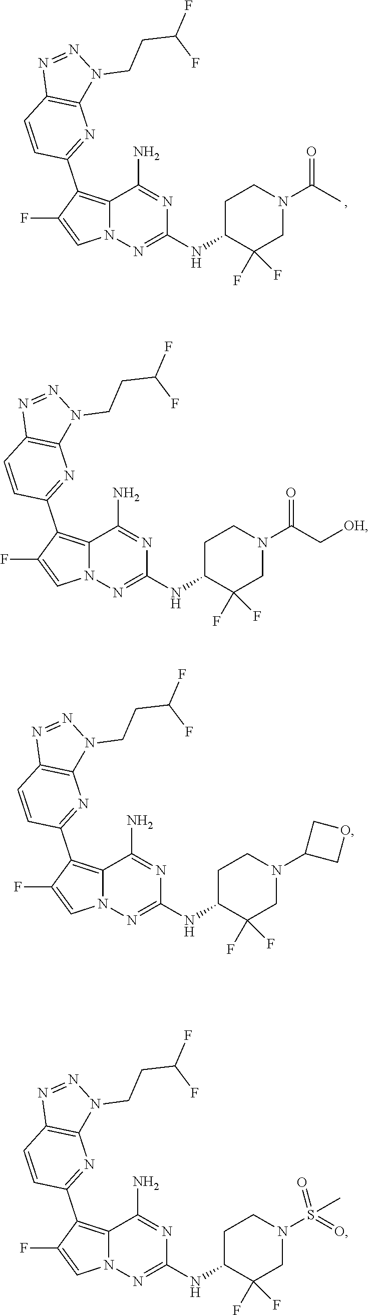 [SMILES: CC(=O)N1CC[C@@H](Nc2nc(N)c3c(-c4ccc5nnn(CCC(F)F)c5n4)c(F)cn3n2)C(F)(F)C1 ; Nc1nc(N[C@@H]2CCN(C(=O)CO)CC2(F)F)nn2cc(F)c(-c3ccc4nnn(CCC(F)F)c4n3)c12 ; Nc1nc(N[C@@H]2CCN(C3COC3)CC2(F)F)nn2cc(F)c(-c3ccc4nnn(CCC(F)F)c4n3)c12 ; CS(=O)(=O)N1CC[C@@H](Nc2nc(N)c3c(-c4ccc5nnn(CCC(F)F)c5n4)c(F)cn3n2)C(F)(F)C1] 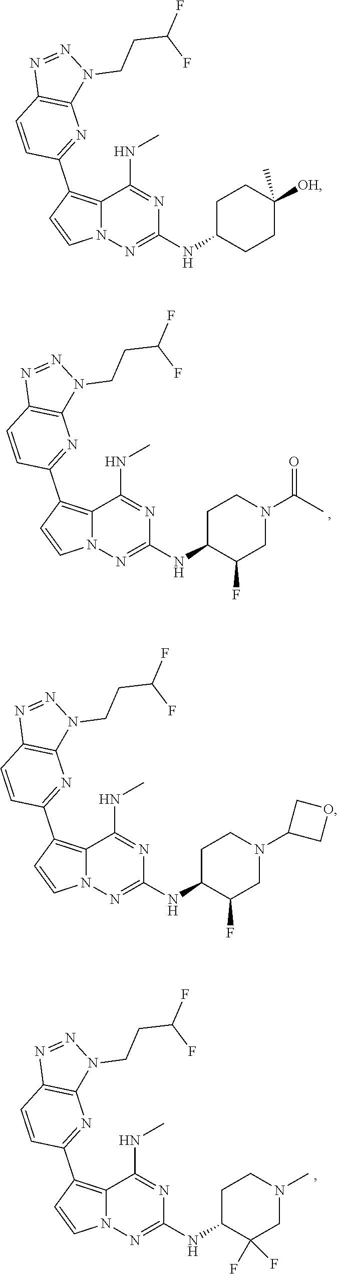 [SMILES: CNc1nc(N[C@H]2CC[C@](C)(O)CC2)nn2ccc(-c3ccc4nnn(CCC(F)F)c4n3)c12 ; CNc1nc(N[C@H]2CCN(C(C)=O)C[C@H]2F)nn2ccc(-c3ccc4nnn(CCC(F)F)c4n3)c12 ; CNc1nc(N[C@H]2CCN(C3COC3)C[C@H]2F)nn2ccc(-c3ccc4nnn(CCC(F)F)c4n3)c12 ; CNc1nc(N[C@@H]2CCN(C)CC2(F)F)nn2ccc(-c3ccc4nnn(CCC(F)F)c4n3)c12] 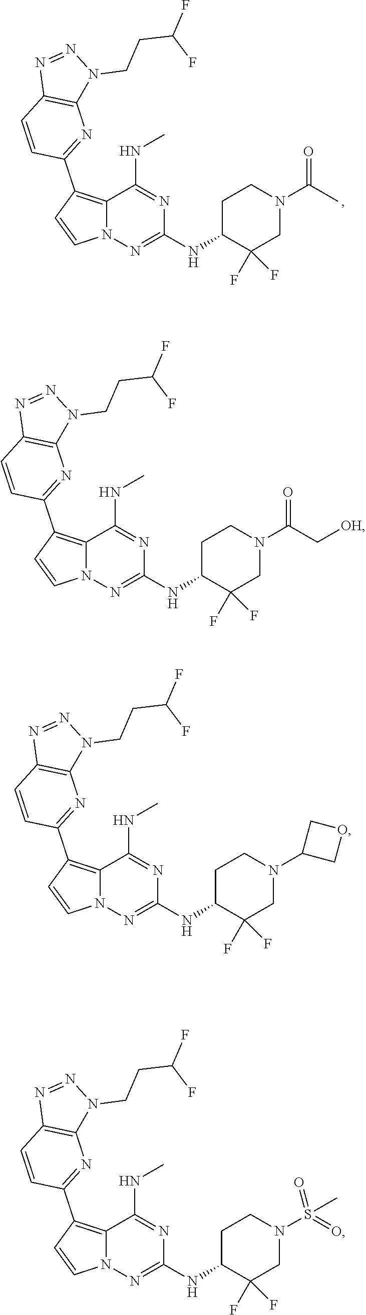 [SMILES: CNc1nc(N[C@@H]2CCN(C(C)=O)CC2(F)F)nn2ccc(-c3ccc4nnn(CCC(F)F)c4n3)c12 ; CNc1nc(N[C@@H]2CCN(C(=O)CO)CC2(F)F)nn2ccc(-c3ccc4nnn(CCC(F)F)c4n3)c12 ; CNc1nc(N[C@@H]2CCN(C3COC3)CC2(F)F)nn2ccc(-c3ccc4nnn(CCC(F)F)c4n3)c12 ; CNc1nc(N[C@@H]2CCN(S(C)(=O)=O)CC2(F)F)nn2ccc(-c3ccc4nnn(CCC(F)F)c4n3)c12] 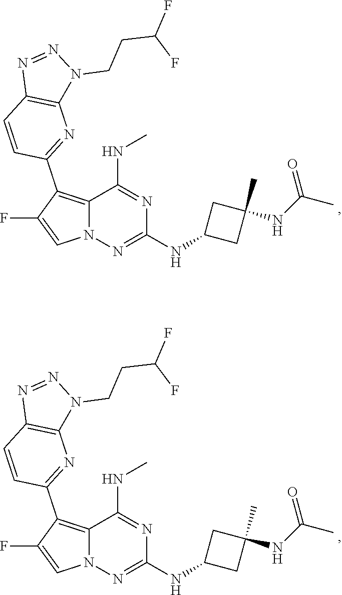 [SMILES: CNc1nc(N[C@H]2C[C@@](C)(NC(C)=O)C2)nn2cc(F)c(-c3ccc4nnn(CCC(F)F)c4n3)c12 ; CNc1nc(N[C@H]2C[C@](C)(NC(C)=O)C2)nn2cc(F)c(-c3ccc4nnn(CCC(F)F)c4n3)c12] 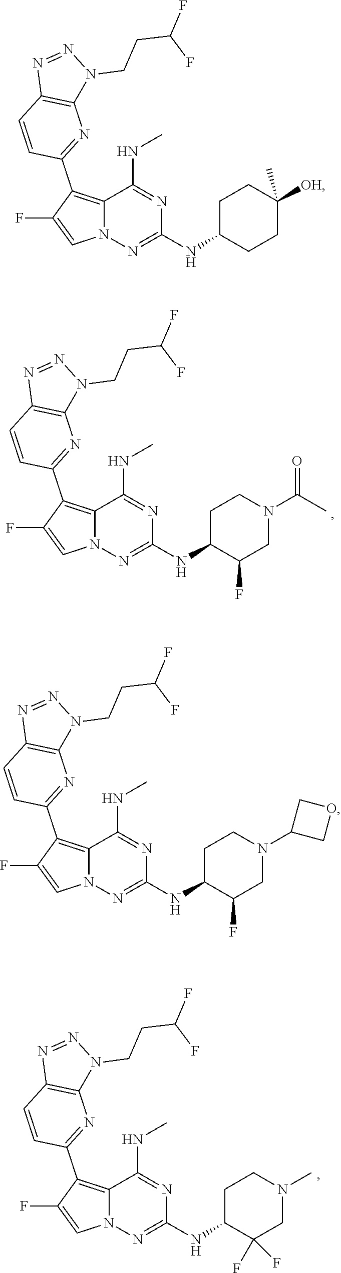 [SMILES: CNc1nc(N[C@H]2CC[C@](C)(O)CC2)nn2cc(F)c(-c3ccc4nnn(CCC(F)F)c4n3)c12 ; CNc1nc(N[C@H]2CCN(C(C)=O)C[C@H]2F)nn2cc(F)c(-c3ccc4nnn(CCC(F)F)c4n3)c12 ; CNc1nc(N[C@H]2CCN(C3COC3)C[C@H]2F)nn2cc(F)c(-c3ccc4nnn(CCC(F)F)c4n3)c12 ; CNc1nc(N[C@@H]2CCN(C)CC2(F)F)nn2cc(F)c(-c3ccc4nnn(CCC(F)F)c4n3)c12] 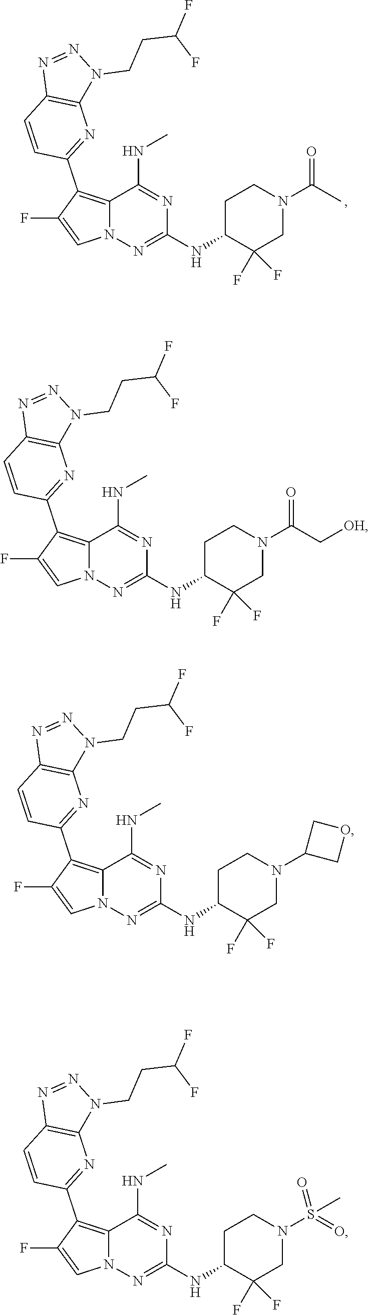 [SMILES: CNc1nc(N[C@@H]2CCN(C(C)=O)CC2(F)F)nn2cc(F)c(-c3ccc4nnn(CCC(F)F)c4n3)c12 ; CNc1nc(N[C@@H]2CCN(C(=O)CO)CC2(F)F)nn2cc(F)c(-c3ccc4nnn(CCC(F)F)c4n3)c12 ; CNc1nc(N[C@@H]2CCN(C3COC3)CC2(F)F)nn2cc(F)c(-c3ccc4nnn(CCC(F)F)c4n3)c12 ; CNc1nc(N[C@@H]2CCN(S(C)(=O)=O)CC2(F)F)nn2cc(F)c(-c3ccc4nnn(CCC(F)F)c4n3)c12] 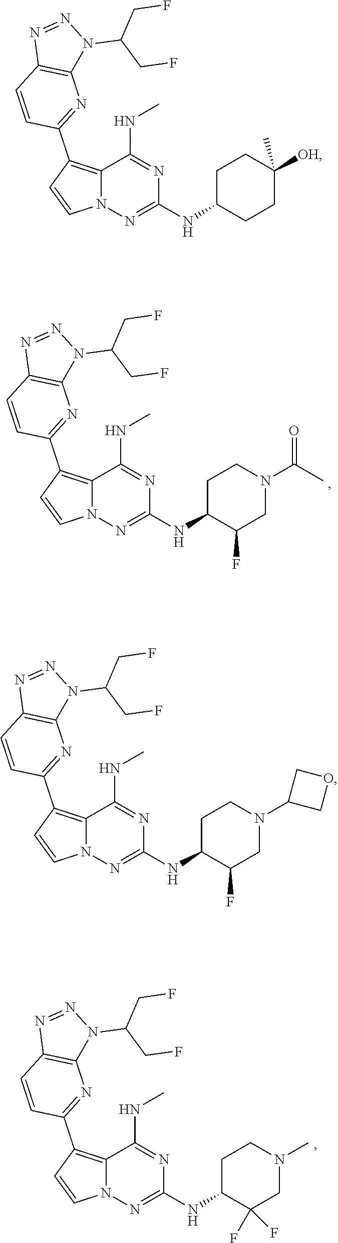 [SMILES: CNc1nc(N[C@H]2CC[C@](C)(O)CC2)nn2ccc(-c3ccc4nnn(C(CF)CF)c4n3)c12 ; CNc1nc(N[C@H]2CCN(C(C)=O)C[C@H]2F)nn2ccc(-c3ccc4nnn(C(CF)CF)c4n3)c12 ; CNc1nc(N[C@H]2CCN(C3COC3)C[C@H]2F)nn2ccc(-c3ccc4nnn(C(CF)CF)c4n3)c12 ; CNc1nc(N[C@@H]2CCN(C)CC2(F)F)nn2ccc(-c3ccc4nnn(C(CF)CF)c4n3)c12] 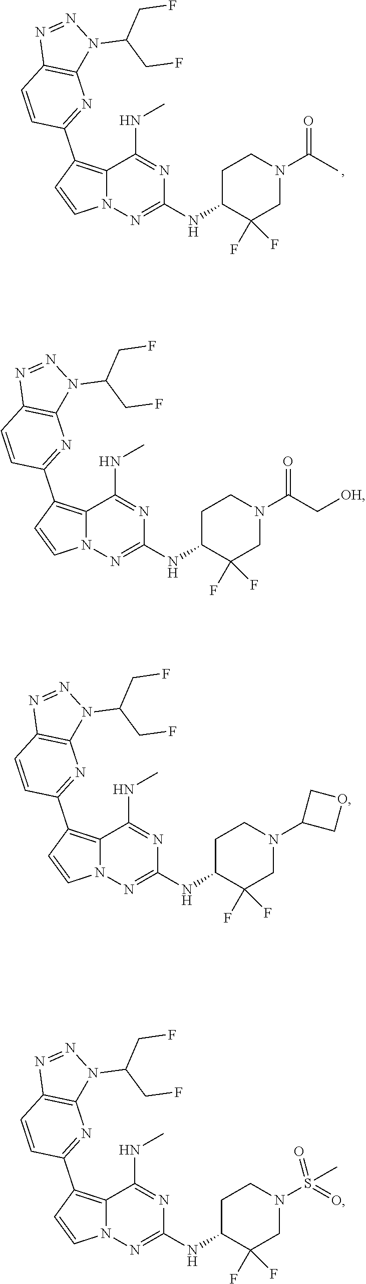 [SMILES: CNc1nc(N[C@@H]2CCN(C(C)=O)CC2(F)F)nn2ccc(-c3ccc4nnn(C(CF)CF)c4n3)c12 ; CNc1nc(N[C@@H]2CCN(C(=O)CO)CC2(F)F)nn2ccc(-c3ccc4nnn(C(CF)CF)c4n3)c12 ; CNc1nc(N[C@@H]2CCN(C3COC3)CC2(F)F)nn2ccc(-c3ccc4nnn(C(CF)CF)c4n3)c12 ; CNc1nc(N[C@@H]2CCN(S(C)(=O)=O)CC2(F)F)nn2ccc(-c3ccc4nnn(C(CF)CF)c4n3)c12] 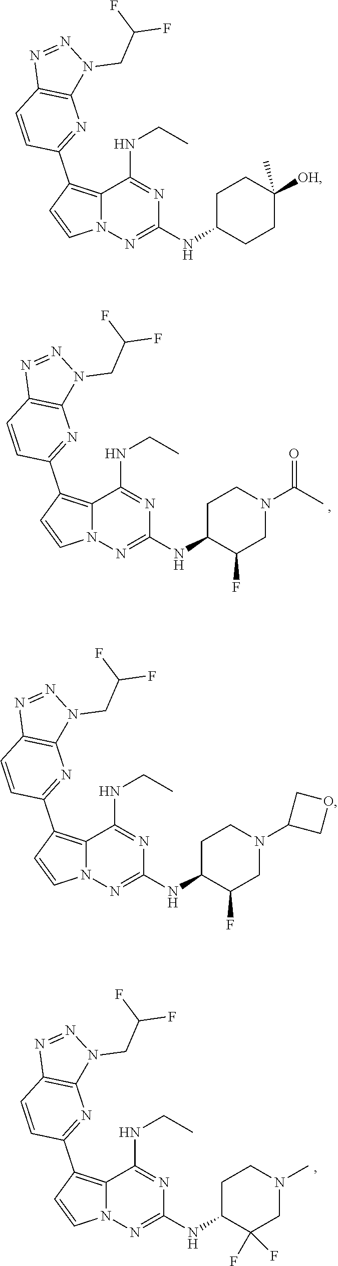 [SMILES: CCNc1nc(N[C@H]2CC[C@](C)(O)CC2)nn2ccc(-c3ccc4nnn(CC(F)F)c4n3)c12 ; CCNc1nc(N[C@H]2CCN(C(C)=O)C[C@H]2F)nn2ccc(-c3ccc4nnn(CC(F)F)c4n3)c12 ; CCNc1nc(N[C@H]2CCN(C3COC3)C[C@H]2F)nn2ccc(-c3ccc4nnn(CC(F)F)c4n3)c12 ; CCNc1nc(N[C@@H]2CCN(C)CC2(F)F)nn2ccc(-c3ccc4nnn(CC(F)F)c4n3)c12] 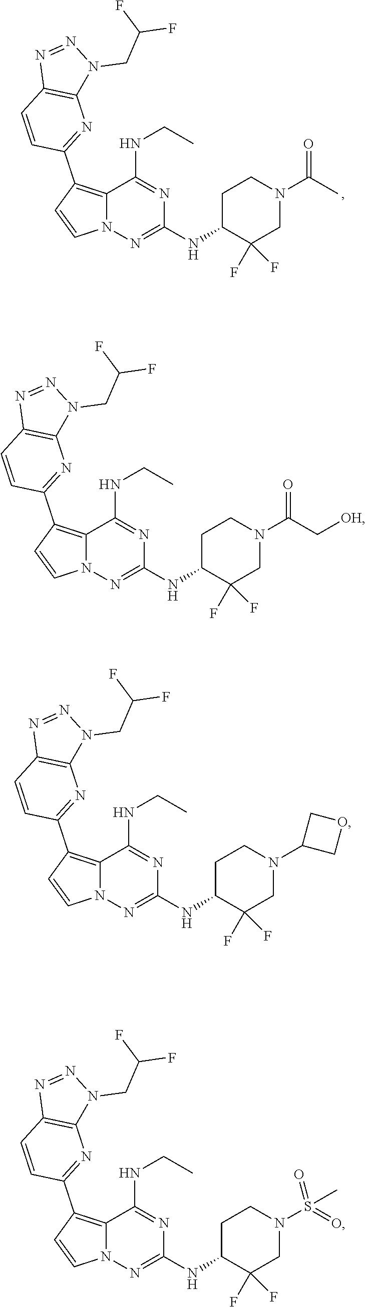 [SMILES: CCNc1nc(N[C@@H]2CCN(C(C)=O)CC2(F)F)nn2ccc(-c3ccc4nnn(CC(F)F)c4n3)c12 ; CCNc1nc(N[C@@H]2CCN(C(=O)CO)CC2(F)F)nn2ccc(-c3ccc4nnn(CC(F)F)c4n3)c12 ; CCNc1nc(N[C@@H]2CCN(C3COC3)CC2(F)F)nn2ccc(-c3ccc4nnn(CC(F)F)c4n3)c12 ; CCNc1nc(N[C@@H]2CCN(S(C)(=O)=O)CC2(F)F)nn2ccc(-c3ccc4nnn(CC(F)F)c4n3)c12] 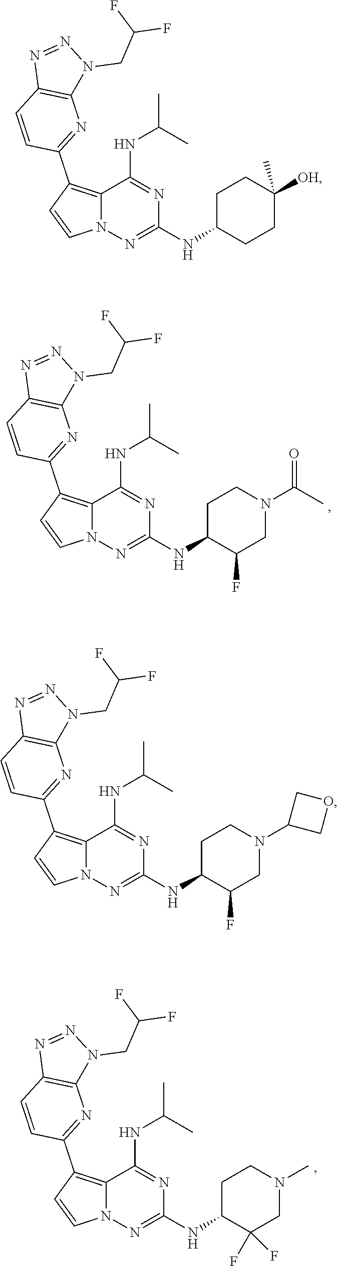 [SMILES: CC(C)Nc1nc(N[C@H]2CC[C@](C)(O)CC2)nn2ccc(-c3ccc4nnn(CC(F)F)c4n3)c12 ; CC(=O)N1CC[C@H](Nc2nc(NC(C)C)c3c(-c4ccc5nnn(CC(F)F)c5n4)ccn3n2)[C@H](F)C1 ; CC(C)Nc1nc(N[C@H]2CCN(C3COC3)C[C@H]2F)nn2ccc(-c3ccc4nnn(CC(F)F)c4n3)c12 ; CC(C)Nc1nc(N[C@@H]2CCN(C)CC2(F)F)nn2ccc(-c3ccc4nnn(CC(F)F)c4n3)c12] 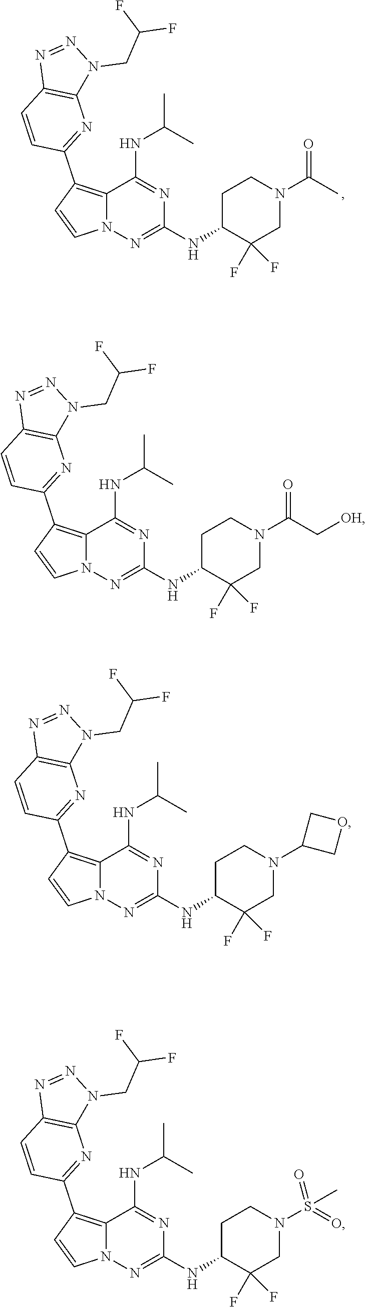 [SMILES: CC(=O)N1CC[C@@H](Nc2nc(NC(C)C)c3c(-c4ccc5nnn(CC(F)F)c5n4)ccn3n2)C(F)(F)C1 ; CC(C)Nc1nc(N[C@@H]2CCN(C(=O)CO)CC2(F)F)nn2ccc(-c3ccc4nnn(CC(F)F)c4n3)c12 ; CC(C)Nc1nc(N[C@@H]2CCN(C3COC3)CC2(F)F)nn2ccc(-c3ccc4nnn(CC(F)F)c4n3)c12 ; CC(C)Nc1nc(N[C@@H]2CCN(S(C)(=O)=O)CC2(F)F)nn2ccc(-c3ccc4nnn(CC(F)F)c4n3)c12] 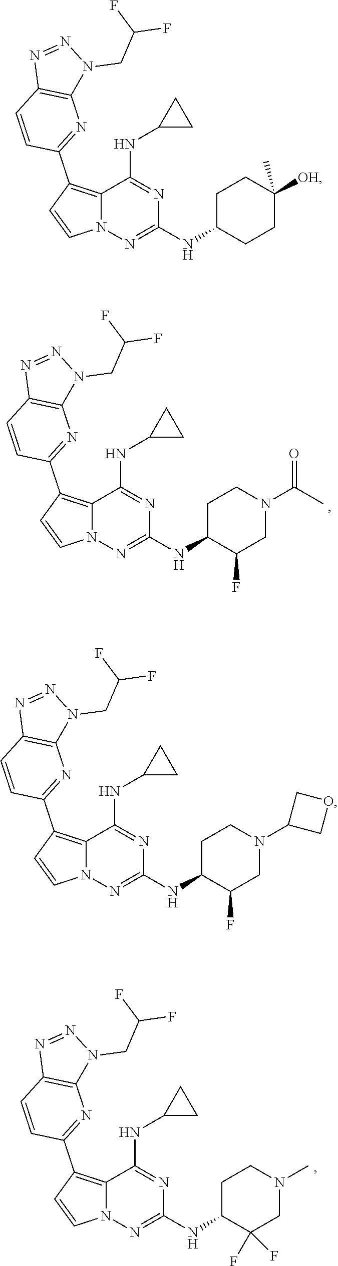 [SMILES: C[C@]1(O)CC[C@H](Nc2nc(NC3CC3)c3c(-c4ccc5nnn(CC(F)F)c5n4)ccn3n2)CC1 ; CC(=O)N1CC[C@H](Nc2nc(NC3CC3)c3c(-c4ccc5nnn(CC(F)F)c5n4)ccn3n2)[C@H](F)C1 ; FC(F)Cn1nnc2ccc(-c3ccn4nc(N[C@H]5CCN(C6COC6)C[C@H]5F)nc(NC5CC5)c34)nc21 ; CN1CC[C@@H](Nc2nc(NC3CC3)c3c(-c4ccc5nnn(CC(F)F)c5n4)ccn3n2)C(F)(F)C1] 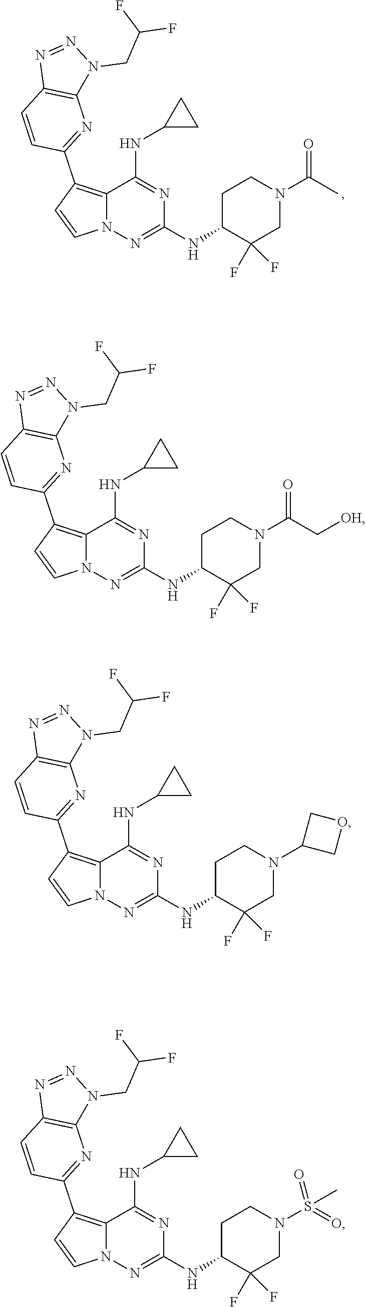 [SMILES: CC(=O)N1CC[C@@H](Nc2nc(NC3CC3)c3c(-c4ccc5nnn(CC(F)F)c5n4)ccn3n2)C(F)(F)C1 ; O=C(CO)N1CC[C@@H](Nc2nc(NC3CC3)c3c(-c4ccc5nnn(CC(F)F)c5n4)ccn3n2)C(F)(F)C1 ; FC(F)Cn1nnc2ccc(-c3ccn4nc(N[C@@H]5CCN(C6COC6)CC5(F)F)nc(NC5CC5)c34)nc21 ; CS(=O)(=O)N1CC[C@@H](Nc2nc(NC3CC3)c3c(-c4ccc5nnn(CC(F)F)c5n4)ccn3n2)C(F)(F)C1]
or a pharmaceutically acceptable salt thereof.

17. A pharmaceutical composition comprising a therapeutically effective amount of a compound of claim 1, or a pharmaceutically acceptable salt thereof, and a pharmaceutically acceptable excipient.

18. A pharmaceutical composition comprising a therapeutically effective amount of a compound of claim 16, or a pharmaceutically acceptable salt thereof, and a pharmaceutically acceptable excipient.

19. A method of treating a disorder or disease in a patient, wherein the disorder or disease is selected from the group consisting of: a neurological disorder, diabetes, and cancer, the method comprising administering to the patient a therapeutically effective amount of a compound of claim 1, or a pharmaceutically acceptable salt.

20. The method of claim 19, wherein the cancer is selected from the group consisting of: brain tumors, glioblastoma, ovarian, breast, head and neck squamous cell carcinoma, hepatocellular carcinoma, pancreatic cancer, acute lymphoblastic leukemia, acute megakaryoblastic leukemia, and chronic myeloid leukemia.

21. The method of claim 19, wherein the disorder or disease is a neurological disorder, wherein the neurological disorder is selected from the group consisting of: Alzheimer's Disease, Amyotrophic Lateral Sclerosis, CDKL5 Deficiency Disorder, Down Syndrome, Frontotemporal Dementia with Parkinsonism-17 (FTDP-17), Lewy body dementia, Parkinson's Disease, Pick's Disease, and additional diseases with pronounced neurodegeneration such as Autism, Dementia, Epilepsy, Huntington's Disease, Multiple Sclerosis; diseases and disorders associated with acquired brain injury such as Chronic Traumatic Encephalopathy, Traumatic Brain Injury, Tumor, Stroke, Pick disease, progressive supranuclear palsy, corticobasal degeneration, argyrophilic grain disease, globular glial tauopathies, primary age-related tauopathy, neurofibrillary tangle dementia, chronic traumatic encephalopathy (CTE), frontotemporal lobar degeneration with tau inclusions (FTLD-tau), and aging-related tau astrogliopathy.

22. The method of claim 21, wherein the disorder or disease is Alzheimer's disease.

23. The method of claim 19, wherein the patient is a human.4-AMINOPYRROLO[2,1-F][1,2,4]TRIAZINES AND PREPARATION AND USES THEREOF
